(19) United States
(12) Patent Application Publication
(10) Pub. No.: US 2006/0223866 A1
(43) Pub. Date: Oct. 5, 2006

(54) Methods and compositions for modulating sphingosine-1-phosphate (S1P) receptor activity

(75) Inventors: Ghotas Evindar, Waltham, MA (US); Hongfeng Deng, Acton, MA (US); Barry Morgan, Franklin, MA (US)

Correspondence Address: LAHIVE & COCKFIELD, 28 STATE STREET, BOSTON, MA, 02109, US

(73) Assignee: Praecis Pharmaceuticals, Inc., Waltham, MA (US)

(21) Appl. No.: 11/349,069

(22) Filed: Feb. 6, 2006

Related U.S. Application Data
(63) Continuation-in-part of application No. 11/204,266, filed on Aug. 12, 2005.
(60) Provisional application No. 60/601,232, filed on Aug. 13, 2004.
(60) Provisional application No. 60/646,436, filed on Jan. 21, 2005.

(51) Int. Cl. A61K 31/426; A61K 31/421; A61K 31/4172; A61K 31/415

(52) U.S. Cl. 514/365; 514/374; 514/400; 514/406; 548/335.5; 548/201; 548/215

(57) ABSTRACT

The present invention relates to compounds which modulate the activity of the S1P1 receptor, the use of these compounds for treating conditions associated with signaling through the S1P1 receptor, and pharmaceutical compositions comprising these compounds.

### RELATED APPLICATIONS

This application is a continuation-in-part of U.S. patent application Ser. No. 11/204266, filed on Aug. 12, 2005, which claims the benefit of and priority to U.S. Provisional Patent Application Ser. No. 60/601232, filed Aug. 13, 2004, and U.S. Provisional Patent Application Ser. No. 60/646436, filed Jan. 21, 2005. This application relates to PCT application No. PCT/US05/28914, filed Aug. 12, 2005.

The entire contents of each of the aforementioned patent applications are hereby incorporated herein by reference.

### BACKGROUND OF THE INVENTION

The sphingosine-1-phosphate (SIP) receptors 1-5 constitute a family of seven transmembrane G-protein coupled receptors. These receptors, referred to as S1P1 to S1P5, are activated via binding by sphingosine-1-phosphate, which is produced by the sphingosine kinase-catalyzed phosphorylation of sphingosine. S1P receptors are cell surface receptors involved in a variety of cellular processes, including cell proliferation and differentiation, cell survival, cell invasion, lymphocyte trafficking, and cell migration. Sphingosine-1-phosphate is found in plasma and a variety of other tissues, and exerts autocrine and paracrine effects, including regulating the secretion of growth factors.

Administration of S1P to an animal results in sequestration of lymphocytes into the lymph nodes and Peyers patches without causing lymphocyte depletion. This activity, which is of potential utility in treating diseases or conditions associated with inappropriate immune response, including transplant rejection and autoimmnune diseases, is believed to proceed via activation of the S1P1 receptor. Administration of S1P in vivo also has negative effects, including hypotension and bradycardia, which are believed to be due to signaling through one or more of the other S1P receptors, i.e. S1P2 to S1P5. Accordingly, there is a great need in the art for compounds which are potent and selective agonists of the S1P1 receptor.

### SUMMARY OF THE INVENTION

The present invention relates to compounds which modulate the activity of the S1P1 receptor, the use of these compounds for treating conditions associated with signaling through the S1P1 receptor, and pharmaceutical compositions comprising these compounds.

Accordingly, in one embodiment, the invention pertains, at least in part, to compounds of Formula I: 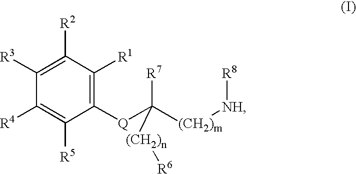
wherein:

wherein one of R³ and R⁴ is C₄-C₂₀-alkyl, C₄-C₂₀-alkoxy; an oxaalkyl, thiaalkyl or azaalkyl group having a chain length of from 4 to 20 atoms, a phenyl or substituted phenyl group, a phenoxy or substituted phenoxy group, a substituted or unsubstituted arylalkyl group, a substituted or unsubstituted arylalkoxy group, a substituted or unsubstituted heteroarylalkyl group; or a substituted or unsubstituted heteroarylalkoxy group; and the other is hydrogen, halogen, cyano, straight chain or branched C₁-C₆-alkyl, straight chain or branched C₁-C₆-alkoxy, straight chain or branched halo-C₁-C₆-alkyl (e.g., trifluoromethyl), straight chain or branched halo-C₁-C₆-alkoxy, C₁-C₆-alkoxy-C₁-C₆-alkyl, hydroxyl-C₁-C₆-alkyl, carboxy-C₁-C₆-alkyl, C₁-C₆-alkyl-SO₂ or N(R)R′, where R and R′ are each independently hydrogen, straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl or C₁-C₆-alkyl-SO₂;

R¹, R², and R⁵ are each independently selected from the group consisting of hydrogen, halogen, cyano, straight chain or branched C₁-C₆-alkyl, straight chain or branched C₁-C₆-alkoxy, straight chain or branched halo-C₁-C₆-alkyl (eg., trifluoromethyl), straight chain or branched halo-C₁-C₆-alkoxy, C₁-C₆-alkoxy-C₁-C₆-alkyl, hydroxyl-C₁-C₆-alkyl, carboxy-C₁-C₆-alkyl, C₁-C₆-alkyl-SO₂ or N(R)R′, where R and R′ are each independently hydrogen, straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl or C₁-C₆-alkyl-SO₂;

Q is —CH₂NR—, —CH₂NR(CO)—, —NH(CO)—, —(CO)NH—, —(CO)—, —O—, —S—, —SO—, —SO₂—, —NRSO₂—, —SO₂—NR— or heteroaryl, where R is hydrogen or straight chain or branched C₁-C₆-alkyl;

R⁶ is —OH, —CO₂R⁹, —CH₂═CH(CO)OR⁹, —OPO₂R¹⁰R¹¹, —OPO₃R¹⁰R¹¹, —CH₂PO₃R¹⁰R¹¹, —OPO₂(S)R¹⁰R¹¹ or —C(Y)(X)PO₃R¹⁰R¹¹, where X is hydroxyl or halide and Y is H or halide; or analogues of other carboxylate, phosphate or phosphonate isosteres not limited to those shown below; R⁹ is H, straight chain or branched C₁-C₆-alkyl, or a substituted or unsubstituted aryl group; R¹⁰ and R¹¹ are each independently H, straight chain or branched C₁-C₆-alkyl, a substituted or unsubstituted aryl group or selected from, but not limited to, the prodrugs listed below:

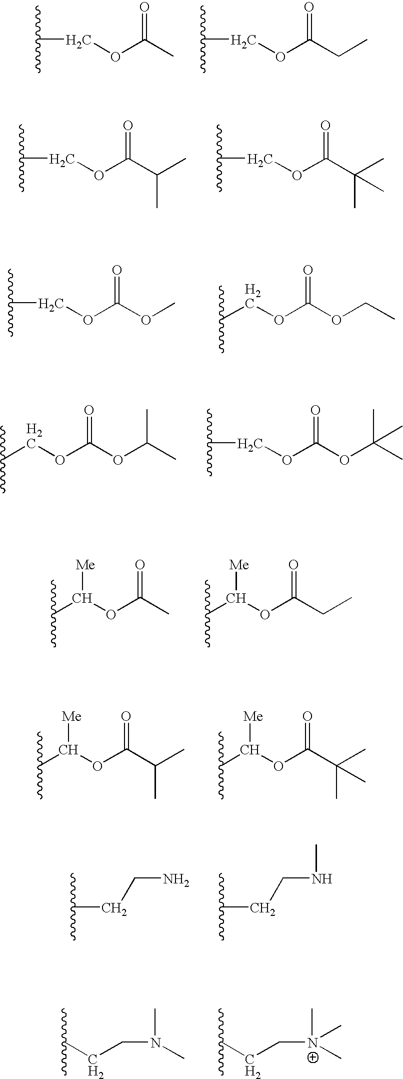

R⁷ is H, C₁-C₆-alkyl, hydroxy-C-C₆-alkyl, aryl, or together with R8 form a C₂-C₅-alkylene or a C₂-C₅-alkenylene group;

R⁸ is H or C₁-C₆-alkyl; and

m and n are each, independently, an integer from 0 to 3; provided that when R⁴ is C₄-C₂₀-alkyl, at least one of R¹, R², R³ and R⁵ is not hydrogen; and when R³ is C₄-C₂₀-alkyl, at least one of R¹, R², R⁴ and R⁵ is not hydrogen; and pharmaceutically acceptable salts thereof.

In another embodiment, the invention provides a compound of Formula II: 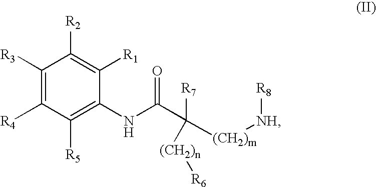
wherein one of R³ and R⁴ is C₄-C₂₀-alkyl, C₄-C₂₀-alkoxy; an oxaalkyl, thiaalkyl or azaalkyl group having a chain length of from 4 to 20 atoms, a phenyl or substituted phenyl group, a phenoxy or substituted phenoxy group, a substituted or unsubstituted arylalkyl group, a substituted or unsubstituted arylalkoxy group, a substituted or unsubstituted heteroarylalkyl group; or a substituted or unsubstituted heteroarylalkoxy group; and the other is hydrogen, halogen, cyano, straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl, C₁-C₆-alkyl-SO₂ or N(R)R′, where R and R′ are each independently hydrogen, straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl or C₁-C₆-alkyl-SO₂;

R¹, R², and R⁵ are each independently selected from the group consisting of hydrogen, halogen, cyano, straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl, C₁-C₆-alkyl-SO₂ or N(R)R′, where R and R′ are each independently hydrogen, straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl or C₁-C₆-alkyl-SO₂;

Q is —CH₂NR—, —CH₂NR(CO)—, —NH(CO)—, —(CO)NH—, —(CO)—, —O—, —S—, —SO—, —SO₂—, —NRSO₂—, —SO₂—NR— or heteroaryl, where R is hydrogen or straight chain or branched C₁-C₆-alkyl;

R⁶is —OH, —CO₂R⁹, —CH₂═CH(CO)OR⁹, —OPO₂R¹⁰R¹¹, —OPO₃R¹⁰R¹¹, —CH₂PO₃R¹⁰R¹¹, —OPO₂(S)R¹⁰R¹¹ or —C(Y)(X)PO₃R¹⁰R¹¹, where X is hydroxyl or halide and Y is H or halide; or analogues of other carboxylate, phosphate or phosphonate isosteres not limited to those shown below; R⁹ is H, straight chain or branched C₁-C₆-alkyl, or a substituted or unsubstituted aryl group; R¹⁰ and R¹¹ are each independently H, straight chain or branched C₁-C₆-alkyl, a substituted or unsubstituted aryl group or selected from, but not limited to, the prodrugs listed below:

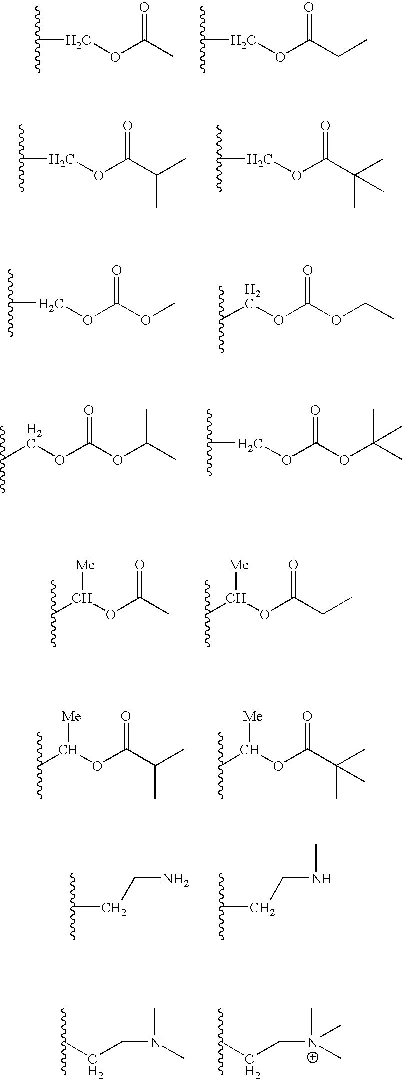

R⁷is H, C₁-C₆-alkyl, hydroxy-C₁-C₆-alkyl, aryl, or together with R₈ form a C₂-C₅-alkylene or a C₂-C₅-alkenylene group;

R⁸ is H or C₁-C₆-alkyl; and

m and n are each, independently, an integer from 0 to 3;

provided that when R₄ is C₄-C₂₀-alkyl, at least one of R¹, R², R³ and R⁵ is not hydrogen; and when R³ is C₄-C₂₀-alkyl, at least one of R¹, R², R⁴ and R⁵ is not hydrogen; and pharmaceutically acceptable salts thereof.

In another embodiment, the invention provides compounds of Formula III: 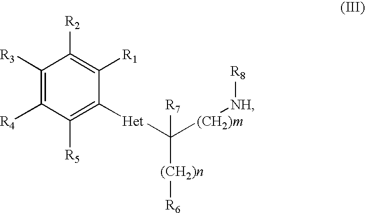
wherein:

Het is heteroaryl group;

R₃ and R₄ are each independently hydrogen, C₄-C₂₀-alkyl group, C₄-C₂₀-alkoxy group or an oxaalkyl, thiaalkyl or azaalkyl group having a chain length of from 4 to 20 atoms; a phenyl or substituted phenyl group, a phenoxy or substituted phenoxy group, a substituted or unsubstituted arylalkyl group, a substituted or unsubstituted arylalkoxy group, a substituted or unsubstituted heteroarylalkyl group; or a substituted or unsubstituted heteroarylalkoxy group;

R₁, R₂, and R₅ are each independently hydrogen, halogen, straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl, C₁-C₆-alkyl-SO₂ or N(R)R′, where R and R′ are each independently hydrogen, straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl or C₁-C₆-alkyl-SO₂;

R⁶ is —OH, —CO₂R⁹, —CH₂═CH(CO)OR⁹, —OPO₂R¹⁰R¹¹, —OPO₃R¹⁰R¹¹, —CH₂PO₃R¹⁰R¹¹, —OPO₂(S)R¹⁰R¹¹ or —C(Y)(X)PO₃R¹⁰R¹¹, where X is hydroxyl or halide and Y is H or halide; or analogues of other carboxylate, phosphate or phosphonate isosteres not limited to those shown below; R⁹ is H, straight chain or branched C₁-C₆-alkyl, or a substituted or unsubstituted aryl group; R¹⁰ and R¹¹ are each independently H, straight chain or branched C₁-C₆-alkyl, a substituted or unsubstituted aryl group or selected from, but not limited to, the prodrugs listed below: 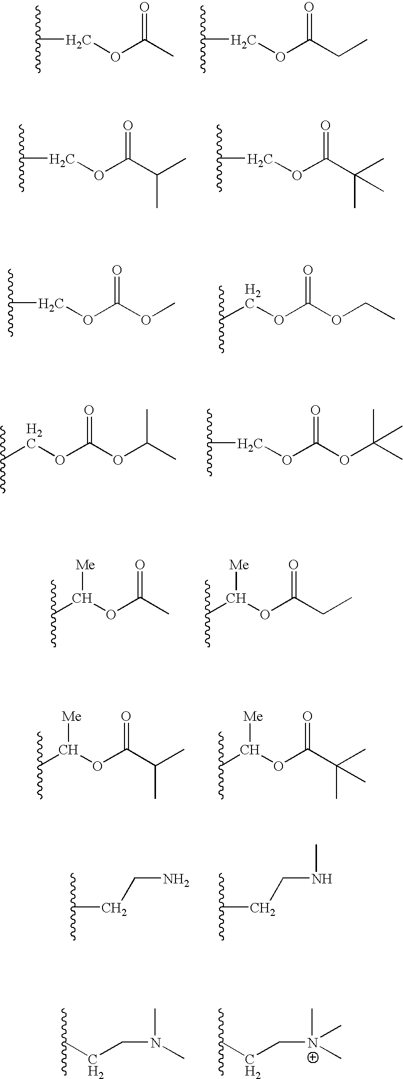
R₇ is H, C₁-C₆-alkyl, hydroxy-C₁-C₆-alkyl, aryl or together with R₈ form a C₂-C₅-alkylene or a C₂-C₅-alkenylene group;
R₈ is H or C₁-C₆-alkyl;
m and n are each, independently, an integer from 0 to 3.
provided that when R⁴ is C₄-C₂₀-alkyl, at least one of R¹, R², R³ and R⁵ is not hydrogen; and when R³ is C₄-C₂₀-alkyl, at least one of R¹, R², R⁴ and R⁵ is not hydrogen; and pharmaceutically acceptable salts thereof.

In an additional embodiment, the invention provides compounds of Formula IV: 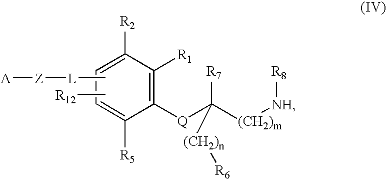
wherein:

L is alkoxy, a covalent bond, substituted or unsubstituted alkyl, alkylcarbonyl, thioether, alkylsulfonyl, alkylcarbonylamino, alkylaminocarbonyl, alkyloxycarbonyl, alkylcarbonyloxy, or substituted or unsubstituted heteroaryl;

Z and A are each independently substituted or unsubstituted aryl, wherein Z and A may be linked by a covalent bond, substituted or unsubstituted alkyl, NH, alkyloxy, O, thioether, S, aminocarbonyl, carbonylamino, carbonyloxy, or oxycarbonyl;

R¹, R², R⁵ and R¹² are each independently selected from the group consisting of hydrogen, halogen, cyano, substituted or unsubstituted aryl, straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl, C₁-C₆-alkyl-SO₂ or N(R)R′, wherein R and R′ are each independently hydrogen, straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl or C₁-C₆-alkyl-SO₂;

Q is —CH₂NR—, —CH₂NR(CO)—, —NH(CO)—, —(CO)NH—, —(CO)—, —O—, —S—, —SO—, —SO₂—, —NRSO₂—, —SO₂—NR— or heteroaryl, where R is hydrogen or straight chain or branched C₁-C₆-alkyl;

R⁶ is —OH, —CO₂R⁹, —CH₂═CH(CO)OR⁹, —OPO₂R¹⁰R¹¹, —OPO₃R¹⁰R¹¹, —CH₂PO₃R¹⁰R¹¹, —OPO₂(S)R¹⁰R¹¹ or —C(Y)(X)PO₃R¹⁰R¹¹, where X is hydroxyl or halide and Y is H or halide; or analogues of other carboxylate, phosphate or phosphonate isosteres not limited to those shown below; R⁹ is H, straight chain or branched C₁-C₆-alkyl, or a substituted or unsubstituted aryl group; R10 and R11 are each independently H, straight chain or branched C₁-C₆-alkyl, a substituted or unsubstituted aryl group or selected from, but not limited to, the prodrugs listed below:

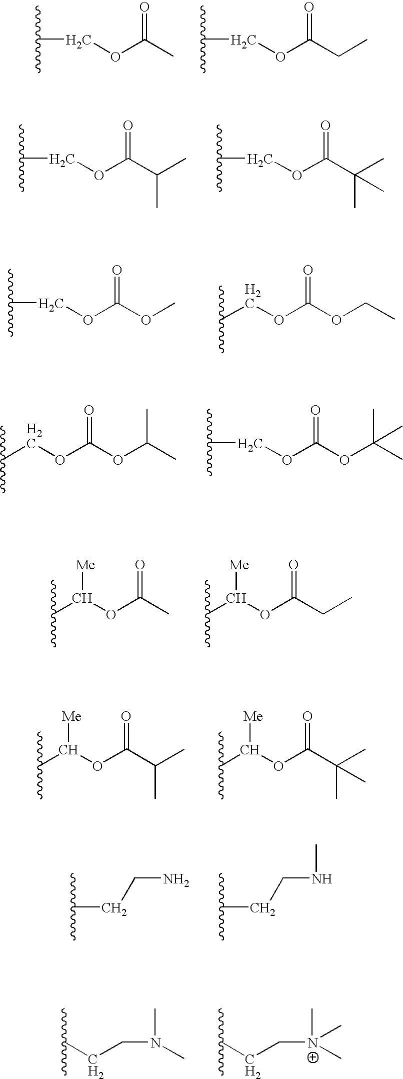

R⁷ is H, C₁-C₆-alkyl, hydroxy-C₁-C₆-alkyl, aryl, or together with R8 form a C₂-C₅-alkylene or a C₂-C₅-alkenylene group;

R⁸ is H or C₁-C₆-alkyl; and

m and n are each, independently, an integer from 0 to 3; provided that when R⁴ is C₄-C₂₀-alkyl, at least one of R¹, R², R³ and R⁵ is not hydrogen; and when R³ is C₄-C₂₀-alkyl, at least one of R¹, R², R⁴ and R⁵ is not hydrogen; and pharmaceutically acceptable salts thereof.

Another embodiment of the invention pertains to a compound of Formula XII: 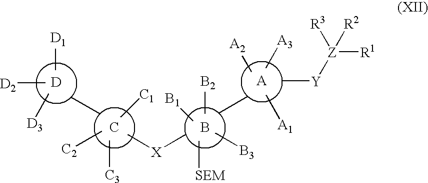
wherein:

SEM represents a selectivity enhancing moiety;

rings A, B, C, D are independently selected from the group consisting of substituted or unsubstituted carbocyclic rings and substituted or unsubstituted heterocyclic rings, which may contain one or more heteroatoms and may be saturated or unsaturated;

A₁, A₂, A₃, B₁, B₂, B₃, C₁, C₂, C₃, D₁, D₂, and D₃ are each independently selected from the group consisting of hydrogen, halogen, cyano, straight chain or branched aliphatic, straight chain or branched alkoxy, straight chain or branched halo-alkyl, straight chain or branched halo-alkoxy, alkoxy-alkyl, hydroxyl-alkyl, carboxy-alkyl, alkyl-SO₂ alkylcarbonyl, thioether, alkylsulfonyl, alkylcarbonylamino, alkylaminocarbonyl, alkyloxycarbonyl, alkylcarbonyloxy, substituted or unsubstituted aryl, substituted or unsubstituted heteroaryl, —OH, —C(O)-alkyl, —C(O)-halo-alkyl, —C(O)O-alkyl, —C(O)O-halo-alkyl, —CONH₂, —CONH-alkyl, —CON-dialkyl, —CONH-halo-alkyl, —CON-halo-dialkyl, -alkyl-CONH-alkyl, -alkyl-CON-dialkyl, halo-alkyl-CONH-alkyl, halo-alkyl-CONH-halo-alkyl, alkyl-CONH-halo-dialkyl, -alkyl-hydroxyl,-alkyl-hydroxyl-alkyl, -halo-alkyl-hydroxyl-alkyl, -alkyl-hydroxyl-halo-alkyl, -halo-alkyl-hydroxyl-halo-alkyl, substituted or unsubstituted alkyl-OR¹⁴, substituted or unsubstituted haloalkyl-OR¹⁴, —OR¹⁴, and N(R)R′; or taken together A₃ and B₃ may form a substituted or unsubstituted carbocyclic ring or substituted or unsubstituted heterocyclic ring, which may contain one or more heteroatoms and may be saturated or unsaturated; or taken together C₂ and D₂ may form a substituted or unsubstituted carbocyclic ring or substituted or unsubstituted heterocyclic ring, which may contain one or more heteroatoms and may be saturated or unsaturated;

R and R′ are each independently selected from the group consisting of hydrogen, cyano, straight chain or branched alkyl, straight chain or branched alkoxy, straight chain or branched halo-alkyl, straight chain or branched halo-alkoxy, alkoxy-alkyl, hydroxyl-alkyl, and carboxy-alkyl; or taken together R and R′ may form a substituted or unsubstituted carbocyclic ring or substituted or unsubstituted or heterocyclic ring, which may contain one or more heteroatoms and may be saturated or unsaturated; or taken together with the N to which they are attached R and R′ may form a moiety selected from the group consisting of substituted straight chain or cyclic guanyl, straight chain or cyclic guanidine, straight chain or cyclic urea, straight chain or cyclic thiourea, straight chain or cyclic carbamate, and straight chain or cyclic thiocarbamate;

Z is independently selected from the group consisting of C or N;

R¹ is a phosphate derivative, a phosphate mimic or a phosphate precursor;

R² and R³ are each independently selected from the group consisting of hydrogen, hydroxyl, halogen, cyano, straight chain or branched alkyl, alkyl-OR⁹, halo-alkyl-OR⁹, alkoxy-OR⁹, alkyl-OC(O)R⁹, halo-alkyl-OC(O)R⁹, alkoxy-OC(O)R⁹, carbocyclic rings, heterocyclic rings which may contain one or more heteroatoms, alkyl-NR⁹R¹⁰, halo-alkyl-NR⁹R¹⁰, and alkoxy-NR⁹R¹⁰, all of which may be optionally substituted with OH, halogen, NHR⁹, NR⁹R¹⁰, straight chain or branched alkoxy, straight chain or branched halo-alkyl, straight chain or branched halo-alkoxy, alkoxy-alkyl, hydroxyl-alkyl, or carboxy-alkyl; or taken together R² and R³ may form a substituted or unsubstituted carbocyclic ring or a substituted or unsubstituted heterocyclic ring, which may contain one or more heteroatoms and may be saturated or unsaturated; or taken together R² and A₁ may form a substituted or unsubstituted C₄-C₁₀ fused carbocyclic ring or substituted or unsubstituted C₄-C₁₀ fused heterocyclic rings, which may contain one or more heteroatoms and may be saturated or unsaturated;

R⁹ and R¹⁰ are independently selected from the group consisting of hydrogen, halogen, cyano, straight chain or branched alkyl, straight chain or branched alkoxy, straight chain or branched halo-alkyl, straight chain and branched halo-alkoxy, —C(O)alkyl, —C(O)NH-alkyl, —C(O)N-dialkyl, —C(O)aryl, —C(O)NH-aryl, —C(O)N-alkyl-aryl, —C(O)N-diaryl, —C(O)heteroaryl, —C(O)NH-heteroaryl, —C(O)N-carbocycle, substituted or unsubstituted carbocyclic rings, and substituted or unsubstituted heterocyclic rings, which may contain one or more heteroatoms and may be saturated or unsaturated; or taken together R⁹ and R¹⁰ may form a substituted or unsubstituted carbocyclic ring or a substituted or unsubstituted heterocyclic ring, which may contain one or more heteroatoms and may be saturated or unsaturated; or R⁹ or R¹⁰ together with A₁ may form a substituted or unsubstituted C₄-C₁₀ fused carbocyclic ring or substituted or unsubstituted C₄-C₁₀ fused heterocyclic rings, which may contain one or more heteroatoms and may be saturated or unsaturated;

Y is independently selected from the group consisting of (CR¹¹R¹²)ₙ and (CR¹¹R¹²)ₙNR¹³;

R¹¹, R¹², and R¹³ are independently selected from the group consisting of hydrogen, halogen, cyano, and straight chain or branched alkyl, all of which may be optionally substituted with OH, halogen, straight chain or branched alkoxy, straight chain or branched halo-alkyl, and straight chain and branched halo-alkoxy; or R¹³ may form a 3-8-membered ring together with either R¹¹ or R¹² and the atom to which they are attached;

n is an integer from 0 to 3;

X is selected from the group consisting of

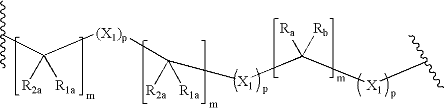

wherein each m is independently selected from an integer between 0 and 6; each p is independently selected from 0 or 1; each X₁ is independently selected from the group consisting of CR¹⁴R¹⁵, NR¹⁴, S, and O, —S(O), —S(O)₂, —OS(O)₂O—, —C(O), C(OH), —C(O)O—, a substituted or unsubstituted aromatic, a substituted or unsubstituted heteroaromatic, and any combination thereof, in any orientation; each Rₐ and R_{b} are independently selected from the group consisting of hydrogen, cyano, halogen, alkyl, halo-alkyl, —OH, —CO—, straight chain or branched alkoxy, straight chain or branched halo-alkyl, straight chain or branched halo-alkoxy, alkoxy-alkyl, hydroxyl-alkyl, -alkyl-hydroxyl, -alkyl-hydroxyl-alkyl, -halo-alkyl-hydroxyl-alkyl, -alkyl-hydroxyl-halo-alkyl, -halo-alkyl-hydroxyl-halo-alkyl, carboxy-alkyl, alkyl-SO₂, alkylcarbonyl, thioether, alkylsulfonyl, alkylcarbonylamino, alkylaminocarbonyl, alkyloxycarbonyl, alkylcarbonyloxy, substituted or unsubstituted aryl, and substituted or unsubstituted heteroaryl, all of which may be optionally substituted with OH, halogen, e.g., fluoro, straight chain or branched alkoxy, straight chain or branched halo-alkyl, straight chain and branched halo-alkoxy, alkoxy-alkyl, hydroxyl-alkyl, carboxy-alkyl, substituted or unsubstituted carbocyclic rings, and substituted or unsubstituted heterocyclic rings, which may contain one or more heteroatoms and may be saturated or unsaturated; or each Rₐ and R_{b} may form a 3-10-membered ring together with the carbon to which they are both attached;

each R₁ₐ and R₂ₐ are independently selected from the group consisting of hydrogen, cyano, halogen, alkyl, halo-alkyl, —OH, —CO—, straight chain or branched alkoxy, straight chain or branched halo-alkyl, straight chain or branched halo-alkoxy, alkoxy-alkyl, hydroxyl-alkyl, -alkyl-hydroxyl, -alkyl-hydroxyl-alkyl, -halo-alkyl-hydroxyl-alkyl, -alkyl-hydroxyl-halo-alkyl, -halo-alkyl-hydroxyl-halo-alkyl, carboxy-alkyl, alkyl-SO₂, alkylcarbonyl, thioether, alkylsulfonyl, alkylcarbonylamino, alkylaminocarbonyl, alkyloxycarbonyl, alkylcarbonyloxy, substituted or unsubstituted aryl, and substituted or unsubstituted heteroaryl, all of which may be optionally substituted with OH, halogen, e.g., fluoro, straight chain or branched alkoxy, straight chain or branched halo-alkyl, straight chain and branched halo-alkoxy, alkoxy-alkyl, hydroxyl-alkyl, carboxy-alkyl, substituted or unsubstituted carbocyclic rings, and substituted or unsubstituted heterocyclic rings, which may contain one or more heteroatoms and may be saturated or unsaturated; or each R₁ₐ and R₂ₐ may form a 3-10-membered ring together with the carbon to which they are both attached; and

each R¹⁴ and R¹⁵ is independently selected from the group consisting of hydrogen, halogen, cyano, straight chain or branched alkyl, straight chain or branched alkoxy, straight chain or branched halo-alkyl, straight chain or branched halo-alkoxy, alkoxy-alkyl, hydroxyl-alkyl, alkyl-SO₂, and carboxy-alkyl; or each R¹⁴ or R¹⁵ may form a 3-8-membered ring together with B₁, Rₐ, R_{b}, R₁ₐ, or R₂ₐ and the atoms to which they are attached.

In yet another embodiment, the invention includes a method for treating a subject suffering from a sphingosine 1-phosphate associated disorder. The method includes administering to the subject an effective amount of a compound of the invention, e.g., a compound of any of Formulae I-XLVII or otherwise described herein, such as a compound of Formula XII, such that the subject is treated for the sphingosine 1-phosphate associated disorder.

In a further embodiment, the invention pertains to a pharmaceutical composition comprising a therapeutically effective amount of a compound of the invention, e.g., a compound of any of Formulae I-XLVII or otherwise described herein, such as a compound of Formula XII, and a pharmaceutically acceptable carrier.

In another embodiment, the invention relates to a method for treating a subject suffering from a sphingosine 1-phosphate associated disorder, comprising administering to a subject an effective amount of a compound of Formula XII, such that the subject is treated for a sphingosine 1-phosphate associated disorder.

In an additional embodiment, the present invention is directed to a method of selectively treating a sphingosine 1-phosphate associated disorder, comprising administering to a subject an effective amount of a compound of Formula XII, such that the subject is selectively treated for a sphingosine 1-phosphate associated disorder.

Another embodiment of the invention is a method of selectively treating a sphingosine 1-phosphate associated disorder, comprising administering to a subject a compound, such that the subject is selectively treated for a sphingosine 1-phosphate associated disorder by a compound of Formula XII.

In yet another embodiment, the invention is directed to a packaged pharmaceutical composition comprising a container holding a therapeutically effective amount of a compound of Formula XII; and instructions for using the compound to treat a sphingosine 1-phosphate associated disorder in a subject.

Another embodiment of the invention relates to a packaged pharmaceutical composition comprising a container holding a therapeutically effective amount of a compound of Formula XII, and instructions for using the compound to selectively treat a sphingosine 1-phosphate associated disorder in a subject.

### BRIEF DESCRIPTION OF THE DRAWING

FIG. 1 is a graph showing the results of the lymphopenia assay for certain compounds of the invention.

### DETAILED DESCRIPTION OF THE INVENTION

The compounds provided by the present invention are modulators of the S1P1 receptor, e.g., agonists or antagonists of the S1P1 receptor. In particular embodiments, the compounds are selective agonists of the S1P1 receptor, e.g., containing a selectivity enhancing moiety (SEM). In addition to the S1P1 modulator compounds, the invention also provides pharmaceutical compositions comprising these compounds and methods of using these compounds for treating a condition associated with an inappropriate immune response, such as transplant rejection or an autoimmune disease.

Definitions

The term “selectivity enhancing moiety (SEM),” as used herein, refers to one or more moieties that provide an enhancement in the selectivity of the compound to which they are attached for the S1P1 receptor, as compared to the compound not containing the moiety or moieties. The SEM confers selectivity to the compound to which it is attached for the S1P1 receptor as compared to, for example, the S1P2 to S1P5 receptors. The enhancement conferred to a compound by the SEM may be measured by, for example, determining the binding specificity of a compound for the S1P1 receptor and one or more of the other S1P receptors or by monitoring the phosphorylation of a compound, e.g., in vivo, wherein enhancement conferred to a compound by the SEM may be in the form of increased binding and/or increased phosphorylation. Without wishing to be bound by theory, it is believed that the enhancement of the selectivity for the S1P1 receptor may be the result of an alteration in the overall conformation of the compound (e.g., due to local or global conformational changes); the electronic properties of the compound (e.g., resulting in altered binding properties locally or globally); or the lypophilicity of the compound.

In certain embodiments, the SEM is selected from, but not limited to, a group consisting of halogen (e.g., F, Cl, and Br), cyano, straight chain or branched C₁-C₆-alkyl, straight chain or branched C₁-C₆-alkoxy, straight chain or branched halo-C₁-C₆-alkyl (e.g., CF₃), straight chain or branched halo-C₁-C₆-alkoxy, C₁-C₆-alkoxy-C₁-C₆-alkyl, hydroxyl-C₁-C₆-alkyl, carboxy-C₁-C₆-alkyl, C₁-C₆-alkyl-SO₂ or N(R)R′, wherein R and R′ are each independently hydrogen, straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl or C₁-C₆-alkyl-SO₂; alkylcarbonyl, thioether, alkylsulfonyl, alkylcarbonylamino, alkylaminocarbonyl, alkyloxycarbonyl, alkylcarbonyloxy, substituted or unsubstituted aromatic , substituted or unsubstituted heteroaromatic, any straight chain or branched alkylene, straight chain or branched alkenyl, straight chain or branched alkynyl, straight chain or branched alkenylene, arylalkyl, alkylaryl, alkylene-aryl, alkenyl-aryl, alkynyl-aryl or alkenylene-aryl group. In a particular embodiment, the SEM is selected from the group consisting of —F, —Cl, —Br, —I, -halo-alkyl, e.g., CF₃, —CN, —COR¹⁸, —CH₂OR¹⁸, —CHFOR¹⁸, CF₂OR¹⁸, —OR, —N(R¹⁸)R¹⁹, aryl, alkylcarbonyl, thioether, alkylsulfonyl, alkylcarbonylamino, alkylaminocarbonyl, alkyloxycarbonyl, alkylcarbonyloxy, substituted or unsubstituted aromatic, substituted or unsubstituted heteroaromatic, straight chain or branched alkyl, straight chain or branched alkenyl, straight chain or branched alkynyl, straight chain or branched alkenyl, arylalkyl, alkylaryl, alkenyl-aryl, and alkynyl-aryl, groups; wherein R¹⁸ and R¹⁹ are each independently selected from hydrogen, straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl or C₁-C₆-alkyl-SO₂. In certain embodiments, the SEM is not hydrogen.

In certain embodiments of the compounds of the invention, the SEM is selected from a group consisting of cyano, straight chain or branched C₁-C₆-alkyl, straight chain or-branched C₁-C₆-alkoxy, straight chain or branched halo-C₁-C₆-alkyl (e.g., CF₃), straight chain or branched halo-C₁-C₆-alkoxy, C₁-C₆-alkoxy-C₁-C₆-alkyl, hydroxyl-C₁-C₆-alkyl, carboxy-C₁-C₆-alkyl, C₁-C₆-alkyl-SO₂ or N(R)R′, wherein R and R′ are each independently hydrogen, straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl or C₁-C₆-alkyl-SO₂; alkylcarbonyl, thioether, alkylsulfonyl, alkylcarbonylamino, alkylaminocarbonyl, alkyloxycarbonyl, alkylcarbonyloxy, substituted or unsubstituted aromatic, substituted or unsubstituted heteroaromatic, any straight chain or branched alkylene, straight chain or branched alkenyl, straight chain or branched alkynyl, straight chain or branched alkenylene, arylalkyl, alkylaryl, alkylene-aryl, alkenyl-aryl, alkynyl-aryl or alkenylene-aryl group.

In certain embodiments of the compounds of the invention, the SEM is selected from a group consisting of cyano, straight chain or branched halo-C₁-C₆-alkyl (e.g., CF₃), straight chain or branched halo-C₁-C₆-alkoxy, C₁-C₆-alkoxy-C₁-C₆-alkyl, hydroxyl-C₁-C₆-alkyl, carboxy-C₁-C₆-alkyl, C₁-C₆-alkyl-SO₂ or N(R)R′, wherein R and R′ are each independently hydrogen, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl or C₁-C₆-alkyl-SO₂; alkylcarbonyl, thioether, alkylsulfonyl, alkylcarbonylamino, alkylaminocarbonyl, alkyloxycarbonyl, alkylcarbonyloxy, substituted or unsubstituted aromatic, substituted or unsubstituted heteroaromatic, any straight chain or branched alkylene, straight chain or branched alkenyl, straight chain or branched alkynyl, straight chain or branched alkenylene, arylalkyl, alkylaryl, alkylene-aryl, alkenyl-aryl, alkynyl-aryl or alkenylene-aryl group.

In a specific embodiment, the SEM is a haloalkyl, e.g., CF₃, CF₂CF₃, CF₂CF₂CF₃, CFHCF₃, CH₂CF₃, CH₂CH₂CF₃, CHCl₂, and CH₂Cl.

In certain embodiments, the SEM may possess a selectivity enhancing orientation (SEO). The term “selectivity enhancing orientation” or “SEO,” as used herein, refers to the relative selectivity enhancement of a compound based on the orientation of the SEM as well as the additional substitutents on the B ring, either alone or in combination with each other. In particular, the SEO may result from the orientation of the SEM on the B ring to which it is attached, e.g., in relation to the A ring and the X moiety attached to the B ring. In a specific embodiment, the SEM is ortho to the X substituent, e.g., on the B ring of Formula XII. In another specific embodiment, the SEM is meta to the attachment site of the A ring, e.g., on the B ring of Formula XII. In another specific embodiment, the X substituent is para to the attachment site of the A substituent, e.g., on the B ring of Formula XII.

The term “phosphate precursor,” as used herein, refers to substituent moieties, e.g., in Formula XII, that may be directly phosphorylated in vivo, or which may be cleaved in vivo to reveal a moiety that may then be phosphorylated in vivo. In certain embodiments, the phosphate precursor may be L₁-O—H or L₁-O—L₂, wherein L₁ is a linking moiety and L₂ is a labile moiety. Exemplary embodiments of the phosphate precursor, include but are not limited to -alkyl-OH, -halo-alkyl-OH, alkoxy-OH, -alkyl-OCOR⁴, -halo-alkyl-OCOR⁴, -alkoxy-OCOR⁴, -alkyl-OC(O)NR⁴R⁵, -halo-alkyl-OC(O)NHR⁴R⁵, -alkoxy-OC(O)NR⁴R⁵, —(CH₂)_{q}CO₂R⁶, and —(CH₂)ₙCH₂═CHC(O)OR⁶, wherein

q is an integer between 0 and 4;

R⁴ and R⁵ are independently selected from the group consisting of hydrogen, straight chain or branched C₁-C₆-alkyl, all of which may be optionally substituted with OH, halogen, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl, substituted or unsubstituted C₃-C₁₀ carbocyclic rings, and substituted or unsubstituted C₃-C₁₀ heterocyclic rings, which may contain one or more heteroatoms and may be saturated or unsaturated; and

R⁶ is selected from the group consisting of hydrogen, straight chain or branched C₁-C₆-alkyl, straight chain or branched halo-C₁-C₆-alkyl, substituted or unsubstituted aryl group, and a prodrug derivatizing moiety (PDM).

The “linking moiety,” may contain 1-8 atoms or may be a bond, and serves as the connection point through which the phosphate mimic, phosphate derivative, or phosphate precursor substituent moieties are linked to the remaining structure of the compounds of the invention. In certain embodiments, the linking moiety may include, but is not limited to, substituted or unsubstituted alkyl (e.g., methylene chains), substituted or unsubstituted alkenyl (e.g., n-alkenes), substituted or unsubstituted alkynyl, substituted or unsubstituted halo-alkyl, substituted or unsubstituted alkoxy, and substituted or unsubstituted halo-alkoxy. In specific embodiments, the linking moiety may be carbonyl derivatized.

The language “labile moiety” refers to a moiety that is subject to cleavage, e.g., by hydrolysis or enzymatic degradation. In certain embodiments, the labile moiety is an ester moiety, which may result in a carboxylate or hydroxyl derivative, depending on the orientation of the ester functionality in the molecule prior to cleavage.

The term “prodrug derivatizing moiety (PDM)” refers to a moiety that derivatizes the compounds of the invention, resulting in a prodrug. Such prodrugs are art-recognized, e.g., in the art of formulating compounds, and are often used to mask particular functionalities that are excessively reactive in vivo. In certain embodiments the PDM is selected from the group consisting of:

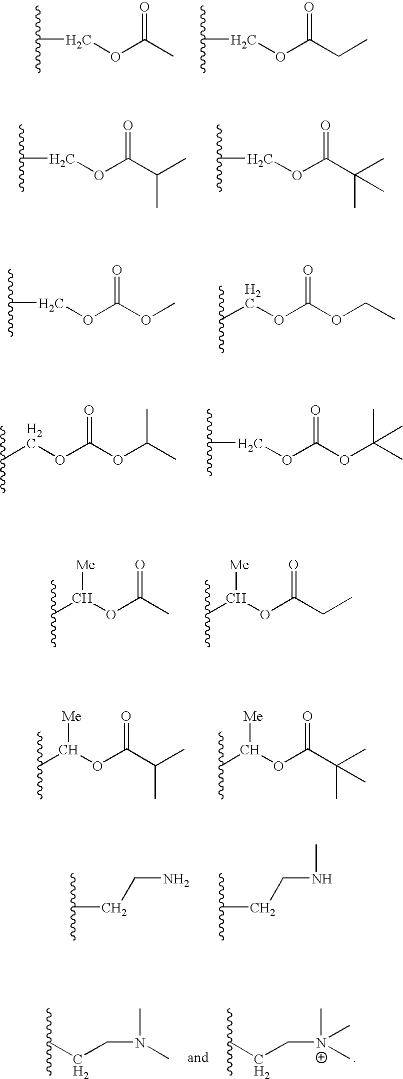

The term “phosphate derivative” refers to substituent moieties, e.g., in Formula XII, that contain a phosphate or phosphate ester group. When a compound of the invention containing a phosphate derivative is administered to a subject, the compound may act as is in vivo or the phosphate derivative (within the compound) may be cleaved and then re-phosphorylated in vivo leading to an active compound. In certain embodiments, the phosphate derivative may be selected from the group consisting of —(CH₂)_{q}OPO₂R⁷R⁸, —(CH₂)_{q}OPO₃R⁷R⁸, and —(CH₂)_{q}OPO₂(S)R⁷R⁸, wherein

q is an integer between 0 and 4; and

R⁷ and R⁸ are each independently selected from the group consisting of hydrogen, straight chain or branched C₁-C₆-alkyl, straight chain or branched halo-C₁-C₆-alkyl, substituted or unsubstituted aryl group, and a prodrug derivatizing moiety (PDM).

The language “phosphate mimic” refers to substituent moieties, e.g., in Formula XII, in which a phosphate substrate has been replaced with a non-hydrolyzable functional group, resulting in a moiety that mimics the structural and/or electronic attributes of a phosphate or phosphate ester moiety. In certain embodiments, the phosphate mimic is -L₁-Z₂, wherein L₁ is a linking moiety and Z₂ is a non-hydrolyzable moiety bonded, e.g., covalently bonded, to L₁. In certain embodiments, the phosphate mimic is selected from the group consisting of —(CH₂)_{q}CH₂PO₃R⁷R⁸, and —(CH₂)_{q}C(Y₁)(Y₂)PO₃R⁷R⁸, wherein

q is an integer between 0 and 4;

Y₁ and Y₂ are independently selected from the group consisting of hydrogen, straight chain or branched C₁-C₆-alkyl, all of which may be optionally substituted with OH, halogen, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl, substituted or unsubstituted C₃-C₁₀ carbocyclic rings, and substituted or unsubstituted C₃-C₁₀ heterocyclic rings, which may contain one or more heteroatoms and may be saturated or unsaturated; and

R⁷ and R⁸ are each independently selected from the group consisting of hydrogen, straight chain or branched C₁-C₆-alkyl, straight chain or branched halo-C₁-C₆-alkyl, substituted or unsubstituted aryl group, and a prodrug derivatizing moiety (PDM).

The language “non-hydrolyzable moiety” is art-recognized, and refers to moieties containing bonds, e.g., C—P bonds, that are not hydrolyzable in vivo.

The term “aliphatic group” includes organic moieties characterized by straight or branched-chains, typically having between 1 and 22 carbon atoms, e.g., between 1 and 8 carbon atoms, e.g., between 1 and 6 carbon atoms. In complex structures, the chains may be branched, bridged, or cross-linked. Aliphatic groups include alkyl groups, alkenyl groups, alkynyl groups, and any combination thereof.

As used herein, “alkyl” groups include saturated hydrocarbons having one or more carbon atoms, e.g., between 1 and 22 carbon atoms, e.g., between 1 and 8 carbon atoms, e.g., between 1 and 6 carbon atoms, including straight-chain alkyl groups (e.g., methyl, ethyl, propyl, butyl, pentyl, hexyl, heptyl, octyl, nonyl, decyl, etc.), cyclic alkyl groups (or “cycloalkyl” or “alicyclic” or “carbocyclic” groups) (e.g., cyclopropyl, cyclopentyl, cyclohexyl, cycloheptyl, cyclooctyl, etc.), branched-chain alkyl groups (isopropyl, tert-butyl, sec-butyl, isobutyl, etc.), and alkyl-substituted alkyl groups (e.g., alkyl-substituted cycloalkyl groups and cycloalkyl-substituted alkyl groups).

In certain embodiments, a straight-chain or branched-chain alkyl group may have 30 or fewer carbon atoms in its backbone, e.g., C₁-C₃₀ for straight-chain or C₃-C₃₀ for branched-chain. In certain embodiments, a straight-chain or branched-chain alkyl group may have 20 or fewer carbon atoms in its backbone, e.g., C₁-C₂₀ for straight-chain or C₃-C₂₀ for branched-chain, and in more particular embodiments 18 or fewer. Likewise, in certain embodiments cycloalkyl groups have from 3-10 carbon atoms in their ring structure, and in more particular embodiments have 3-7 carbon atoms in the ring structure. The term “lower alkyl” refers to alkyl groups having from 1 to 6 carbons in the chain, and to cycloalkyl groups having from 3 to 6 carbons in the ring structure.

In certain embodiments, the alkyl group (e.g., straight, branched, cyclic, and lower alkyl group) is substituted. In particular embodiments, the alkyl group is substituted with one or more halogens, e.g., F. In a specific embodiment, the alkyl group is perfluorinated, e.g., CF₃. Moreover, the alkyl group, in combination with halogen substitution(s) would be understood to be a haloalkyl moiety. Accordingly, and for convenience herein, reference to an alkyl moiety may also incorporate haloalkyl moieties, regardless of whether specific embodiments recited herein are differentiated by explicitly making reference to haloalkly moieties.

Unless the number of carbons is otherwise specified, “lower” as in “lower aliphatic,” “lower alkyl,” “lower alkenyl,” etc. as used herein means that the moiety has at least one and less than about 8 carbon atoms. In certain embodiments, a straight-chain or branched-chain lower alkyl group has 6 or fewer carbon atoms in its backbone (e.g., C₁-C₆ for straight-chain, C₃-C₆ for branched-chain), and in particular embodiments, 4 or fewer. Likewise, in certain embodiments cycloalkyl groups have from 3-8 carbon atoms in their ring structure, and in more particular embodiments have 5 or 6 carbons in the ring structure. The term “C₁-C₆” as in “C₁-C₆ alkyl” means alkyl groups containing 1 to 6 carbon atoms.

Moreover, unless otherwise specified the term alkyl includes both “unsubstituted alkyls” and “substituted alkyls,” the latter of which refers to alkyl groups having substituents replacing one or more hydrogens on one or more carbons of the hydrocarbon backbone. Such substituents may include, for example, alkenyl, alkynyl, halogen, hydroxyl, alkylcarbonyloxy, arylcarbonyloxy, alkoxycarbonyloxy, aryloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclic, alkylaryl, or aromatic (including heteroaromatic) groups.

An “arylalkyl” group is an alkyl group substituted with an aryl group (e.g., phenylmethyl (i.e., benzyl)). An “alkylaryl” moiety is an aryl group substituted with an alkyl group (e.g., p-methylphenyl (i.e., p-tolyl)). The term “n-alkyl⇄ means a straight-chain (i.e., unbranched) unsubstituted alkyl group. An “alkylene” group is a divalent analog of the corresponding alkyl group. Examples of alkylene groups include ethylene (—CH₂CH₂—), propylene (—CH₂CH₂CH₂—), butylene (—CH₂CH₂CH₂CH₂—) and 1-methyethylene (—CH(CH₃)CH₂—). The terms “alkenyl”, “alkynyl” and “alkenylene” refer to unsaturated aliphatic groups analogous to alkyls, but which contain at least one double or triple carbon-carbon bond respectively. Examples of alkenylene groups include ethenylene (“CH═CH—), propenylene (—CH═CHCH₂—), 2-butenylene (—CH₂CH═CHCH₂—) and 1-methyethenylene (—C(CH₃)CH—). Suitable alkenyl and alkynyl groups include groups having 2 to about 12 carbon atoms, preferably from 2 to about 6 carbon atoms.

The term “haloalkyl” describes alkyl moieties that contain one or more of the same or different halogen substituents, e.g., F or Cl. In particular, the term “haloalkyl” includes alkyl moieties comprising one halogen group, alkyl moieties that are perfluorinated, as well as any level of halogenation in between the two extremes. Examplary haloalkyl moieties include, but are not limited to —CF₃, —CH₂F, —CHF₂, —CF₂CF₃, —CF₂CF₃, —CHFCF₃, —CF₂CF₃, —CF₂CF₂H, and —CF₂CHF₂. In addition, haloalkyl groups may be straight chain or branched and may be optionally substituted with additional substituents (i.e., other than the halogen substituents). In particular embodiments, the haloalkyl is —CF₃.

The term —aromatic or aromatic group— and —aryl or aryl group” includes unsaturated and aromatic cyclic hydrocarbons (e.g., benzyl or phenyl) as well as unsaturated and aromatic heterocycles containing one or more rings. Aryl groups may also be fused or bridged with a bond (e.g., biphenyl), alicyclic or heterocyclic rings that are not aromatic so as to form a polycycle (e.g., tetralin). An “arylene” group is a divalent analog of an aryl group.

The term “carbocycle or carbocyclic group” includes any possible saturated or unsaturated closed ring alkyl groups (or “cycloalkyl” or “alicyclic” or “carbocyclic” groups) (e.g., cyclopropyl, cyclopentyl, cyclohexyl, cycloheptyl, cyclooctyl, etc.), any possible C₃-C₁₂ saturated or unsaturated halogenated closed ring alkyl groups, and substituted or unsubstituted aromatic groups. In certain embodiments, the carbocyclic group is a substituted or unsubstituted C₃-C₁₀ carbocyclic ring.

The term “heterocyclic group” includes closed ring structures analogous to carbocyclic groups in which one or more of the carbon atoms in the ring is an element other than carbon, for example, nitrogen, sulfur, or oxygen (e.g. cyclic ethers, lactones, lactams, azitidines). Heterocyclic groups may be saturated or unsaturated. Heterocyclic groups may be halogenated. Additionally, heterocyclic groups (such as pyrrolyl, pyridyl, isoquinolyl, quinolyl, purinyl, and furyl) may have aromatic character, in which case they may be referred to as “heteroaryl” or “heteroaromatic” groups. In certain embodiments, the heterocyclic group is a substituted or unsubstituted C₃-C₁₀ heterocyclic rings.

Unless otherwise stipulated, carbocyclic and heterocyclic (including heteroaryl) groups may also be substituted at one or more constituent atoms. Examples of heteroaromatic and heteroalicyclic groups may have 1 to 3 separate or fused rings with 3 to about 8 members per ring and one or more N, O, or S heteroatoms. In general, the term “heteroatom” includes atoms of any element other than carbon or hydrogen, preferred examples of which include nitrogen, oxygen, sulfur, and phosphorus. Heterocyclic groups may be saturated or unsaturated or aromatic.

Examples of heterocycles include, but are not limited to, acridinyl; azocinyl; benzimidazolyl; benzofuranyl; benzothiofuranyl; benzothiophenyl; benzoxazolyl; benzthiazolyl; benztriazolyl; benztetrazolyl; benzisoxazolyl; benzisothiazolyl; benzimidazolinyl; carbazolyl; 4aH-carbazolyl; carbolinyl; chromanyl; chromenyl; cinnolinyl; decahydroquinolinyl; 2H,6H-1,5,2-dithiazinyl; dihydrofuro[2,3-b]tetrahydrofliran; furanyl; furazanyl; imidazolidinyl; imidazolinyl; imidazolyl; 1H-indazolyl; indolenyl; indolinyl; indolizinyl; indolyl; 3H-indolyl; isobenzofuranyl; isochromanyl; isoindazolyl; isoindolinyl; isoindolyl; isoquinolinyl; isothiazolyl; isoxazolyl; methylenedioxyphenyl; morpholinyl; naphthyridinyl; octahydroisoquinolinyl; oxadiazolyl; 1,2,3-oxadiazolyl; 1,2,4-oxadiazolyl; 1,2,5-oxadiazolyl; 1,3,4-oxadiazolyl; oxazolidinyl; oxazolyl; oxazolidinyl; pyrimidinyl; phenanthridinyl; phenanthrolinyl; phenazinyl; phenothiazinyl; phenoxathiinyl; phenoxazinyl; phthalazinyl; piperazinyl; piperidinyl; piperidonyl; 4-piperidonyl; piperonyl; pteridinyl; purinyl; pyranyl; pyrazinyl; pyrazolidinyl; pyrazolinyl; pyrazolyl; pyridazinyl; pyridooxazole; pyridoimidazole; pyridothiazole; pyridinyl; pyridyl; pyrimidinyl; pyrrolidinyl; pyrrolinyl; 2H-pyrrolyl; pyrrolyl; quinazolinyl; quinolinyl; 4H-quinolizinyl; quinoxalinyl; quinuclidinyl; tetrahydrofuranyl; tetrahydroisoquinolinyl; tetrahydroquinolinyl; tetrazolyl; 6H-1,2,5-thiadiazinyl; 1,2,3-thiadiazolyl; 1,2,4-thiadiazolyl; 1,2,5-thiadiazolyl; 1,3,4-thiadiazolyl; thianthrenyl; thiazolyl; thienyl; thienothiazolyl; thienooxazolyl; thienoimidazolyl; thiophenyl; triazinyl; 1,2,3-triazolyl; 1,2,4-triazolyl; 1,2,5-triazolyl; 1,3,4-triazolyl; and xanthenyl. Preferred heterocycles include, but are not limited to, pyridinyl; furanyl; thienyl; pyrrolyl; pyrazolyl; pyrrolidinyl; imidazolyl; indolyl; benzimidazolyl; 1H-indazolyl; oxazolidinyl; benzotriazolyl; benzisoxazolyl; oxindolyl; benzoxazolinyl; and isatinoyl groups. Also included are fused ring and spiro compounds containing, for example, the above heterocycles.

A common hydrocarbon aryl group is a phenyl group having one ring. Two-ring hydrocarbon aryl groups include naphthyl, indenyl, benzocyclooctenyl, benzocycloheptenyl, pentalenyl, and azulenyl groups, as well as the partially hydrogenated analogs thereof such as indanyl and tetrahydronaphthyl. Exemplary three-ring hydrocarbon aryl groups include acephthylenyl, fluorenyl, phenalenyl, phenanthrenyl, and anthracenyl groups.

Aryl groups also include heteromonocyclic aryl groups, i.e., single-ring heteroaryl groups, such as thienyl, furyl, pyranyl, pyrrolyl, imidazolyl, pyrazolyl, pyridinyl, pyrazinyl, pyrimidinyl, and pyridazinyl groups; and oxidized analogs thereof such as pyridonyl, oxazolonyl, pyrazolonyl, isoxazolonyl, and thiazolonyl groups. The corresponding hydrogenated (i.e., non-aromatic) heteromonocylic groups include pyrrolidinyl, pyrrolinyl, imidazolidinyl, imidazolinyl, pyrazolidinyl, pyrazolinyl, piperidyl and piperidino, piperazinyl, and morpholino and morpholinyl groups.

Aryl groups also include fused two-ring heteroaryls such as indolyl, isoindolyl, indolizinyl, indazolyl, quinolinyl, isoquinolinyl, phthalazinyl, quinoxalinyl, quinazolinyl, cinnolinyl, chromenyl, isochromenyl, benzothienyl, benzimidazolyl, benzothiazolyl, purinyl, quinolizinyl, isoquinolonyl, quinolonyl, naphthyridinyl, and pteridinyl groups, as well as the partially hydrogenated analogs such as chromanyl, isochromanyl, indolinyl, isoindolinyl, and tetrahydroindolyl groups. Aryl groups also include fused three-ring groups such as phenoxathiinyl, carbazolyl, phenanthridinyl, acridinyl, perimidinyl, phenanthrolinyl, phenazinyl, phenothiazinyl, phenoxazinyl, and dibenzofuranyl groups.

Some typical aryl groups include substituted or unsubstituted 5- and 6-membered single-ring groups. In another aspect, each Ar group may be selected from the group consisting of substituted or unsubstituted phenyl, pyrrolyl, furyl, thienyl, thiazolyl, isothiaozolyl, imidazolyl, triazolyl, tetrazolyl, pyrazolyl, oxazolyl, isooxazolyl, pyridinyl, pyrazinyl, pyridazinyl, and pyrimidinyl groups. Further examples include substituted or unsubstituted phenyl, 1-naphthyl, 2-naphthyl, biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl groups.

The term “amine” or “amino,” as used herein, refers to an unsubstituted or substituted moiety of the formula —NR^{a}R^{b}, in which each R^{a} and R^{b} are each independently hydrogen, alkyl, aryl, or heterocyclyl, or each R^{a} and R^{b}, taken together with the nitrogen atom to which they are attached, form a cyclic moiety having from 3 to 8 atoms in the ring. Thus, the term amino includes cyclic amino moieties such as piperidinyl or pyrrolidinyl groups, unless otherwise stated. Thus, the term “alkylamino” as used herein means an alkyl group having an amino group attached thereto. Suitable alkylamino groups include groups having 1 to about 12 carbon atoms, e.g., from 1 to about 6 carbon atoms. The term amino includes compounds or moieties in which a nitrogen atom is covalently bonded to at least one carbon or heteroatom. The term “dialkylamino” includes groups wherein the nitrogen atom is bound to at least two alkyl groups. The term “arylamino” and “diarylamino” include groups wherein the nitrogen is bound to at least one or two aryl groups, respectively. The term “alkylarylamino” refers to an amino group which is bound to at least one alkyl group and at least one aryl group. The term “alkaminoalkyl” refers to an alkyl, alkenyl, or alkynyl group substituted with an alkylamino group. The term “amide” or “aminocarbonyl” includes compounds or moieties which contain a nitrogen atom which is bound to the carbon of a carbonyl or a thiocarbonyl group. The term “azaalkyl” refers to an alkyl group in which one or more —CH₂— units have been replaced by an —N(R)— group, where R is hydrogen or C₁-C₄-alkyl. If an azaalkyl group includes two or more N(R) groups, any two N(R) groups are separated by one or more carbon atoms.

The terms “alkylthio” or “thiaalkoxy” refers to an alkyl group, having a sulfhydryl group attached thereto. Suitable alkylthio groups include groups having 1 to about 12 carbon atoms, e.g., from 1 to about 6 carbon atoms. The term “thiaalky” refers to an alkyl group in which one or more —CH₂” units have been replaced by a sulfur atom. If a thiaalkyl group includes two or more sulfur atoms, any two sulfur atoms are separated by one or more carbon atoms.

The term “alkylcarboxyl” as used herein means an alkyl group having a carboxyl group attached thereto.

The term “alkoxy” as used herein means an alkyl group having an oxygen atom attached thereto. Representative alkoxy groups include groups having 1 to about 12 carbon atoms, e.g., between 1 and 8 carbon atoms, e.g., between 1 and 6 carbon atoms, e.g., methoxy, ethoxy, propoxy, tert-butoxy and the like. Examples of alkoxy groups include methoxy, ethoxy, isopropyloxy, propoxy, butoxy, and pentoxy groups. The alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy, trichloromethoxy, etc., as well as perhalogenated alkyloxy groups. The term “oxaalkyl” refers to an alkyl group in which one or more —CH₂— units have been replaced by an oxygen atom. If an oxaalkyl group includes two or more oxygen atoms, any two oxygen atoms are separated by one or more carbon atoms.

The term “acylamino” includes moieties wherein an amino moiety is bonded to an acyl group. For example, the acylamino group includes alkylcarbonylamino, arylcarbonylamino, carbamoyl and ureido groups.

The terms “alkoxyalkyl”, “alkylaminoalkyl” and “thioalkoxyalkyl” include alkyl groups, as described above, which further include oxygen, nitrogen or sulfur atoms replacing one or more carbons of the hydrocarbon backbone.

The term “carbonyl” or “carboxy” includes compounds and moieties which contain a carbon connected with a double bond to an oxygen atom. Examples of moieties which contain a carbonyl include aldehydes, ketones, carboxylic acids, amides, esters, anhydrides, etc.

The term “ether” or “ethereal” includes compounds or moieties which contain an oxygen atom bonded to two carbon atoms. For example, an ether or ethereal group includes “alkoxyalkyl” which refers to an alkyl, alkenyl, or alkynyl group substituted with an alkoxy group.

The term “nitro” means —NO₂; the term “halogen” or “halogen” or “halo” designates —F, —Cl, —Br or —I; the term “thiol,” “thio,” or “mercapto” means SH; and the term “hydroxyl” or “hydroxy” means —OH.

The term “acyl” refers to a carbonyl group that is attached through its carbon atom to a hydrogen (i.e., a formyl), an aliphatic group (e.g., acetyl), an aromatic group (e.g., benzoyl), and the like. The term “substituted acyl” includes acyl groups where one or more of the hydrogen atoms on one or more carbon atoms are replaced by, for example, an alkyl group, alkynyl group, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

Unless otherwise specified, the chemical moieties of the compounds of the invention, including those groups discussed above, may be “substituted or unsubstituted.” In some embodiments, the term “substituted” means that the moiety has substituents placed on the moiety other than hydrogen (i.e., in most cases, replacing a hydrogen), which allow the molecule to perform its intended function. In certain embodiments, examples of substituents include moieties selected from substituted or unsubstituted aliphatic moieties. In particular embodiments, the exemplary substituents include, but are not limited to, straight or branched alkyl (e.g., C₁-C₅), cycloalkyl (e.g., C₃-C₈), alkoxy (e.g., C₁-C₆), thioalkyl (e.g., C₁-C₆), alkenyl (e.g., C₂-C₆), alkynyl (e.g., C₂-C₆), heterocyclic, carbocyclic, aryl (e.g., phenyl), aryloxy (e.g., phenoxy), arylkyl (e.g., benzyl), aryloxyalkyl (e.g., phenyloxyalkyl), arylacetamidoyl, alkylaryl, heteroaralkyl, alkylcarbonyl and arylcarbonyl or other such acyl group, heteroarylcarbonyl, and heteroaryl groups, as well as (CR′R″) ₀₋₃NR′R″ (e.g., —NH₂), (CR′R″)₀₋₃CN (e.g., —CN), —NO₂, halogen (e.g., —F, —Cl, —Br, or —I), (CR′R″)₀₋₃C(halogen)₃ (e.g., —CF₃), (CR′R″)₀₋₃CH(halogen)₂, (CR′R″)₀₋₃CH₂(halogen), (CR′R″)₀₋₃CONR′R″, (CR′R″)₀₋₃(CNH)NR′R″, (CR′R″)₀₋₃S(O)₁₋₂NR′R″, (CR′R″)₀₋₃CHO, (CR′R″)₀₋₃O(CR′R″)₀₋₃H, (CR′R″)₀₋₃S(O)₀₋₃R′ (e.g., —SO₃H), (CR′R″)₀₋₃O(CR′R″)₀₋₃H (e.g., —CH₂OCH₃ and —OCH₃), (CR′R″)₀₋₃S(CR′R″)₀₋₃H (e.g., —SH and —SCH₃), (CR′R″)₀₋₃H (e.g., —OH), (CR′R″)₀₋₃COR′, (CR′R″)₀₋₃(substituted or unsubstituted phenyl), (CR′R″)₀₋₃(C₃-C₈ cycloalkyl), (CR′R″)₀₋₃CO₂R′ (e.g., —CO₂H), and (CR′R″)₀₋₃OR′ groups, wherein R′ and R″ are each independently hydrogen, a C₁-C₅ alkyl, C₂-C₅ alkenyl, C₂-C₅ alkynyl, or aryl group; or the side chain of any naturally occurring amino acid.

In another embodiment, a substituent may be selected from straight or branched alkyl (e.g., C₁-C₅), cycloalkyl (e.g., C₃-C₈), alkoxy (e.g., C₁-C₆), thioalkyl (e.g., C₁-C₆), alkenyl (e.g., C₂-C₆), alkynyl (e.g., C₂-C₆), heterocyclic, carbocyclic, aryl (e.g., phenyl), aryloxy (e.g., phenoxy), aralkyl (e.g., benzyl), aryloxyalkyl (e.g., phenyloxyalkyl), arylacetamidoyl, alkylaryl, heteroaralkyl, alkylcarbonyl and arylcarbonyl or other such acyl group, heteroarylcarbonyl, or heteroaryl group, (CR′R″)₀₋₁₀NR′R″ (e.g., —NH₂), (CR′R″)₀₋₁₀CN (e.g., —CN), NO₂, halogen (e.g., F, Cl, Br, or I), (CR′R″)₀₋₁₀C(halogen)₃ (e.g., —CF₃), (CR′R″)₀₋₁₀CH(halogen)₂, (CR′R″)₀₋₁₀CH₂(halogen), (CR′R″)₀₋₁₀CONR′R″, (CR′R″)₀₋₁₀(CNH)NR′R″, (CR′R″)₀₋₁₀S(O)₁₋₂NR′R″, (CR′R″)₀₋₁₀CHO, (CR′R″)₀₋₁₀O(CR′R″)₀₋₁₀H, (CR′R″)₀₋₁₀S(O)₀₋₃R′ (e.g., —SO₃H), (CR′R″)₀₋₁₀(CR′R″)₀₋₁₀H (e.g., —CH₂OCH₃ and —OCH₃), (CR′R″)₀₋₁₀S(CR′R″)₀₋₃H (e.g., —SH and —SCH₃), (CR′R″)₀₋₁₀OH (e.g., —OH), (CR′R″)₀₋₁₀COR′, (CR′R″)₀₋₁₀(substituted or unsubstituted phenyl), (CR′R″)₀₋₁₀(C₃-C₈ cycloalkyl), (CR′R″)₀₋₁₀CO₂R′ (e.g., —CO₂H), or (CR′R″)₀₋₁₀OR′ group, or the side chain of any naturally occurring amino acid; wherein R′ and R″ are each independently hydrogen, a C₁-C₅ alkyl, C₂-C₅ alkenyl, C₂-C₅ alkynyl, or aryl group, or R′ and R″ taken together are a benzylidene group or a —(CH₂)₂O(CH₂)₂— group.

It will be understood that “substitution” or “substituted with” includes the implicit proviso that such substitution is in accordance with the permitted valence of the substituted atom and the substituent, and that the substitution results in a stable compound, e.g., which does not spontaneously undergo transformation such as by rearrangement, cyclization, elimination, etc. As used herein, the term “substituted” is meant to include all permissible substituents of organic compounds. In a broad aspect, the permissible substituents include acyclic and cyclic, branched and unbranched, carbocyclic and heterocyclic, aromatic and nonaromatic substituents of organic compounds. The permissible substituents can be one or more. It should further be understood that the substituents described herein may be attached to the moiety that is substituted in any orientation (regardless of whether such attachment orientation is indicated herein by the manner of description, e.g., by a dash)

In some embodiments, a “substituent” may be selected from the group consisting of, for example, halogen, trifluoromethyl, nitro, cyano, C₁-C₆ alkyl, C₂-C₆ alkenyl, C₂-C₆ alkynyl, C₁-C₆ alkylcarbonyloxy, arylcarbonyloxy, C₁-C₆ alkoxycarbonyloxy, aryloxycarbonyloxy, C₁-C₆ alkylcarbonyl, C₁-C₆ alkoxycarbonyl, C₁-C₆ alkoxy, C₁-C₆ alkylthio, arylthio, heterocyclyl, aralkyl, and aryl (including heteroaryl) groups.

Compounds of the Invention

In one embodiment, the invention pertains, at least in part, to compounds of Formula (I): 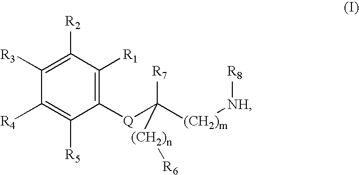
wherein:

one of R₃ and R₄ is selected from the group consisting of C₄-C₂₀-alkyl, C₄-C₂₀-alkoxy; an oxaalkyl, thiaalkyl or azaalkyl group having a chain length of from 4 to 20 atoms, a phenyl or substituted phenyl group, a phenoxy or substituted phenoxy group, a substituted or unsubstituted arylalkyl group, a substituted or unsubstituted arylalkoxy group, a substituted or unsubstituted heteroarylalkyl group; or a substituted or unsubstituted heteroarylalkoxy group; and the other is hydrogen, halogen, cyano, straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl, C₁-C₆-alkyl-SO₂ or N(R)R′, where R and R′ are each independently hydrogen, straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl or C₁-C₆-alkyl-SO₂;

R¹, R², and R⁵ are each independently selected from the group consisting of hydrogen, halogen, cyano, straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl, C₁-C₆-alkyl-SO₂ or N(R)R′, where R and R′ are each independently hydrogen, straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl or C₁-C₆-alkyl-SO₂;

Q is —CH₂NR—, —CH₂NR(CO)—, —NH(CO)—, —(CO)NH—, —(CO)—, —O—, —S—, —SO—, —SO₂—, —NRSO₂—, —SO₂—NR— or heteroaryl, where R is hydrogen or straight chain or branched C₁-C₆-alkyl;

R⁶ is —OH, —CO₂R⁹, —CH₂═CH(CO)OR⁹, —OPO₂R¹⁰R¹¹, —OPO₃R¹⁰R¹¹, —CH₂PO₃R¹⁰R¹¹, —OPO₂(S)R¹⁰R¹¹ or —C(Y)(X)PO₃R¹⁰R¹¹, where X is hydroxyl or halide and Y is H or halide; or analogues of other carboxylate, phosphate or phosphonate isosteres not limited to those shown below; R⁹ is H, straight chain or branched C₁-C₆-alkyl, or a substituted or unsubstituted aryl group; R¹⁰ and R¹¹ are each independently H, straight chain or branched C₁-C₆-alkyl, a substituted or unsubstituted aryl group or selected from, but not limited to, the prodrugs listed below:

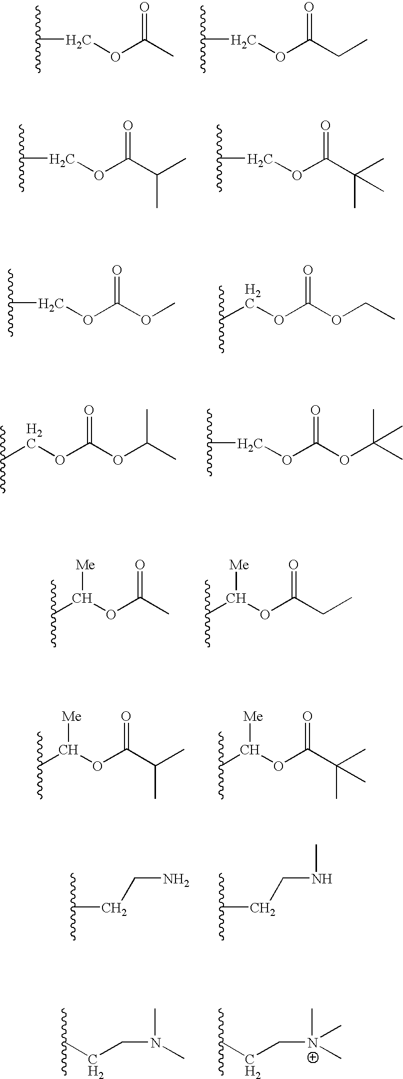

R⁷is H, C₁-C₆-alkyl, hydroxy-C₁-C₆-alkyl, aryl, or together with R⁸ form a C₂-C₅-alkylene or a C₂-C₅-alkenylene group;

R⁸ is H or C₁-C₆-alkyl; and

m and n are each, independently, an integer from 0 to 3;

provided that when R⁴ is C₄-C₂₀-alkyl, at least one of R¹, R², R³ and R⁵ is not hydrogen; and when R³ is C₄-C₂₀-alkyl, at least one of R¹, R², R⁴ and R⁵ is not hydrogen; and pharmaceutically acceptable salts thereof;

provided that when Q is NH(C═O), O, or heteroaryl; R⁶ is OH; n is 1-4; one of R¹, R², R³, R⁴, and R⁵ is C₁-C₁₈ alkyl, C₂-C₁₈ alkenyl, C₂-C₁₈ alkynyl, C₅-C₁₈-alkoxy, (CH₂)₁₋₁₀O(CH₂)₁₋₁₀, C₅-C₁₀(aryl), C₅-C₁₀(aryl)(C₁-C₁₀alkyl), C₅-C₁₀(heteroaryl), C₅-C₁₀(heteroaryl)(C₁-C₁₀alkyl), C₅-C₁₀cycloalkyl, C₅-C₁₀(cycloalkyl)-(C₁-C₅ alkyl), C₅-C₁₀alkoxy(aryl), C₅-C₁₀alkoxy(aryl)(C₁-C₁₀alkyl), C₅-C₁₀alkoxy(heteroaryl), C₅-C₁₀alkoxy(heteroaryl)(C₁-C₁₀alkyl), C₅-C₁₀alkoxy(cycloalkyl), or C₅-C₁₀alkoxy(cycloalkyl)(C₁-C₁₀alkyl); and one of R¹, R², R³, R⁴, and R⁵ is H, halogen, NH₂, C₁-C₆ alkyl, C₁-C₆ alkoxy, C₁-C₆ alkylamino, C₁-C₆ alkylcyano, or C₁-C₆ alkylthio, then R⁸ is not hydrogen;

provided that when Q is heteroaryl; one of R¹, R², R³, R⁴, and R⁵ is alkyl, alkenyl, alkynyl, optionally substituted aryl, optionally substituted heteroaryl, alkyl (optionally substituted aryl), arylalkyl, or arylalkyl (optionally substituted (aryl); R⁸ is hydrogen; n is 1; then R⁶ is not OH;

and provided that when Q is NH(C═O); R⁶ is OH; R¹, R², R³, R⁴, and R⁵ are each independently halogen, hydrogen, amino, or alkyl; then R⁸ is not hydrogen.

In a further embodiment, R¹ is hydrogen. In another further embodiment, R² is hydrogen, alkyl, or halogen (e.g., fluoro, bromo, chloro or iodo).

In another further embodiment, R³ is substituted or unsubstituted alkyl or cycloalkyl group. The alkyl R³ group may be substituted with any substituent that allows the compound of any of Formulae I-XLVII to perform its intended function, e.g., modulate sphingosine 1-phosphate receptor. Examples of such substituents include halogens and hydroxyl groups. Other examples of possible substituents for alkyl R³ groups include substituted or unsubstituted arylthioether, alkylthioether, alkylsulfoxide, arylsulfoxide, arylsulfonyl and alkylsulfonyl groups.

In a further embodiment, R³ is a substituted or unsubstituted alkoxy or cycloalkoxy group (e.g., a C₁-C₂₀ alkoxy group). In a further embodiment, the substituted R³ alkoxy group is substituted with one or more substituted or unsubstituted aryl groups. These aryl groups may further be substituted with any substituent which allows the compounds of the invention to perform their intended function, e.g., modulate sphingosine 1-phosphate 1 receptors. Examples of such substituents include, but are not limited to, alkoxy groups, such as methoxy, ethoxy, and propoxy. These alkoxy groups may further be substituted with any substituents such as halogens, hydroxyl groups, cyano groups, and other substituents described herein.

In another embodiment, R³ is a substituted or unsubstituted aryloxy group, e.g., a substituted or unsubstituted phenoxy group. Furthermore, the phenoxy group may further be substituted with one or more substituents which allow the compound of the invention to perform its intended function. Examples of such substituents include substituted or unsubstituted alkyl or substituted or unsubstituted aryl groups. Examples of aryl groups which may be used to substitute the phenoxy R³ groups include substituted or unsubstituted phenyl groups. Examples of substituents for these phenyl groups include halogens, cyano, alkoxy, alkyl groups, or any of the other possible substituents described herein.

In another embodiment, R³ is a substituted or unsubstituted aryl or heteroaryl group. The substituted aryl or heteroaryl R³ group may further be substituted with one or more halogens, such as fluorine, chlorine, bromine, or iodine. It also may be substituted with any of the other substituents described herein.

In yet another embodiment, R³ is a substituted or unsubstituted alkyl amino carbonyl or a substituted or unsubstituted aryl amino carbonyl. In yet another embodiment, R³ is a substituted or unsubstituted aryl carbonyl, a substituted or unsubstituted alkyl carbonyl, substituted or unsubstituted aryl alkyl carbonyl.

In another embodiment, R⁴ is hydrogen, a cyano group, a substituted or unsubstituted alkyl group, or a substituted or unsubstituted alkoxy group. In a further embodiment, R⁵ is hydrogen, a substituted or unsubstituted alkyl group or a halogen. R⁴ and R⁵ may be substituted with any of the substituents described herein, such that the compound of formula (I) is capable of performing its intended function, e.g., modulate the sphingosine 1-phosphate receptor.

In yet another further embodiment, Q is —NH—CO— or —CO—NH—. In yet another further embodiment, Q is a substituted or unsubstituted aryl group, e.g., phenyl or heteroaryl. Examples of heteroaryl Q groups include pyridyl, indolyl, imidazolyl, furanyl, and other N, S, and O containing heteroaryls.

In another embodiment, Q is a carbonyl or thiocarbonyl group.

In another embodiment, Q is CH₂NR—, —CH₂NR(CO), —NRSO₂— or —SO₂—NR.

In another embodiment, R⁶ is hydrogen, an alkoxy group, or an alkyl ether group. In another further embodiment, R⁶ is a hydroxyl, substituted or unsubstituted alkyl group. R⁶ may be substituted with any substituent which allows the resulting compound of formula (I) to perform its intended function. In another embodiment, R⁶ is a substituted or unsubstituted aryloxy group. Examples of substituted or unsubstituted R⁶ aryloxy group include substituted or unsubstituted phenoxy group. These phenoxy groups may further be substituted with, for example, one or more substituted or unsubstituted alkyl groups.

In yet another embodiment, R⁶ is a phosphate, alkyl phosphate, cycloalkyl phosphate, phosphonate, thiophosphate, alkylthiophosphate, cycloalkylthiophosphate, or thiophosphonate. Other examples of R⁶ include carboxylic acids and substituted and unsubstituted alkyl esters and aryl esters.

In yet another further embodiment, R⁷ is hydrogen, or a substituted or unsubstituted alkyl group. Examples of substituents for alkyl R⁷ groups include hydroxy groups.

In yet another further embodiment, R⁸ is hydrogen, hydroxyl, or substituted or unsubstituted alkyl.

In one embodiment, the invention provides compounds of Formula II:

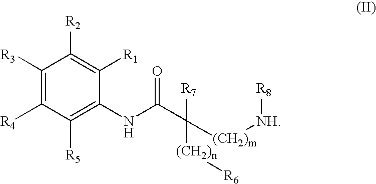

In a first set of compounds of Formula II, R₄ is C₄-C₂₀-alkoxy or an oxaalkyl, thiaalkyl or azaalkyl group having a chain length of from 4 to 20 atoms; a phenyl or substituted phenyl group, a phenoxy or substituted phenoxy group, a substituted or unsubstituted arylalkyl group, a substituted or unsubstituted arylalkoxy group, a substituted or unsubstituted heteroarylalkyl group; or a substituted or unsubstituted heteroarylalkoxy group. R₁, R₂, R₃ and R₅ are each independently selected from the group consisting of hydrogen, halogen, straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl, C₁-C₆-alkyl-SO₂ and N(R)R′, wherein R and R′ are each independently hydrogen, halogen, straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl or C₁-C₆-alkyl-SO₂. R⁶ is —OH, —CO₂R⁹, —CH₂═CH(CO)OR⁹, —OPO₂R¹⁰R¹¹, —OPO₃R¹⁰R¹¹, —CH₂PO₃R¹⁰R¹¹, —OPO₂(S)R¹⁰R¹¹ or —C(Y)(X)PO₃R¹⁰R¹¹, where X is hydroxyl or halide and Y is H or halide; or analogues of other carboxylate, phosphate or phosphonate isosteres not limited to those shown below; R⁹ is H, straight chain or branched C₁-C₆-alkyl, or a substituted or unsubstituted aryl group; R¹⁰ and R¹¹ are each independently H, straight chain or branched C₁-C₆-alkyl, a substituted or unsubstituted aryl group or selected from, but not limited to, the prodrugs listed below: 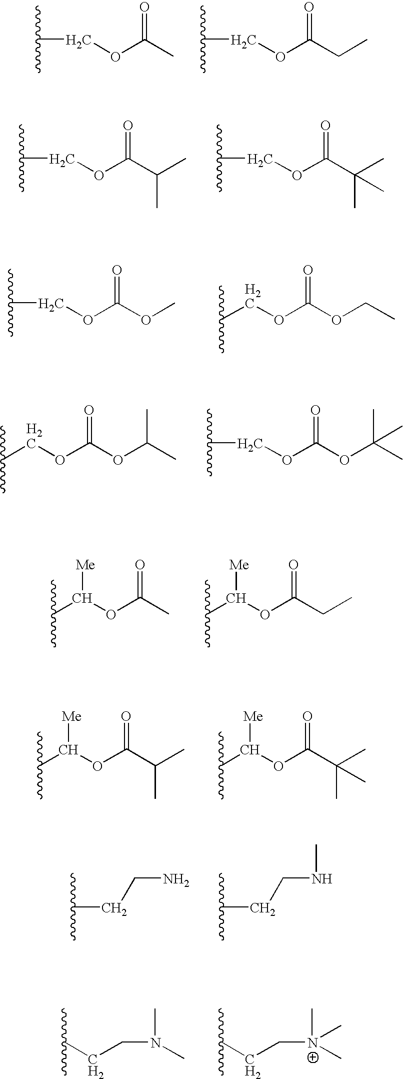
R₇is H, C₁-C₆-alkyl, hydroxy-C₁-C₆-alkyl or aryl. R₈ is H or C₁-C₆-alkyl. R₇ and R₈ can also together form a C₂-C₅-alkylene or a C₂-C₅-alkenylene group; m and n are each, independently, an integer from 0 to 3; provided that when R⁴ is C₄-C₂₀-alkyl, at least one of R¹, R², R³ and R⁵ is not hydrogen; and when R³ is C₄-C₂₀-alkyl, at least one of R¹, R², R⁴ and R⁵ is not hydrogen; and pharmaceutically acceptable salts thereof.

In a second set of compounds of Formula II, R₃ is C₄-C₂₀-alkoxy or an oxaalkyl, thiaalkyl or azaalkyl group having a chain length of from 4 to 20 atoms; a phenyl or substituted phenyl group, a phenoxy or substituted phenoxy group, a substituted or unsubstituted arylalkyl group, a substituted or unsubstituted arylalkoxy group, a substituted or unsubstituted heteroarylalkyl group; or a substituted or unsubstituted heteroarylalkoxy group. R₁, R₂, R₄ and R₅ are each independently selected from the group consisting of hydrogen, halogen, straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl, C₁-C₆-alkyl-SO₂ or N(R)R′, wherein R and R′ are each independently hydrogen, halogen, straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl or C₁-C₆-alkyl-SO₂. R⁶ is —OH, —CO₂R⁹, —CH₂═CH(CO)OR⁹, —OPO₂R¹⁰R¹¹, —OPO₃R¹⁰R¹¹, —CH₂PO₃R¹⁰R¹¹, —OPO₂(S)R¹⁰R¹¹ or —C(Y)(X)PO₃R¹⁰R¹¹, where X is hydroxyl or halide and Y is H or halide; or analogues of other carboxylate, phosphate or phosphonate isosteres not limited to those shown below; R⁹ is H, straight chain or branched C₁-C₆-alkyl, or a substituted or unsubstituted aryl group; R¹⁰ and R¹¹ are each independently H, straight chain or branched C₁-C₆-alkyl, a substituted or unsubstituted aryl group or selected from, but not limited to, the prodrugs listed below: 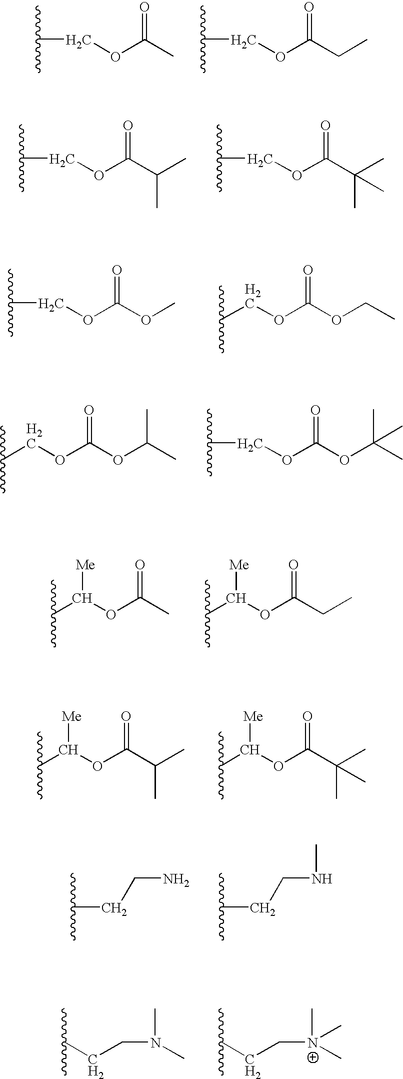
R₇ is H, C₁-C₆-alkyl, hydroxy-C₁-C₆-alkyl or aryl. R₈ is H or C₁-C₆-alkyl. R₇ and R₈ can also together form a C₂-C₅-alkylene or a C₂-C₅-alkenylene group; m and n are each, independently, an integer from 0 to 3, provided that when R⁴ is C₄-C₂₀-alkyl, at least one of R¹, R², R³ and R⁵ is not hydrogen; and when R³ is C₄-C₂₀-alkyl, at least one of R¹, R², R⁴ and R⁵ is not hydrogen; and pharmaceutically acceptable salts thereof.

In a third set of compounds of Formula II, R₃ is C₄-C₂₀-alkyl and R₁, R₂, R₄ and R₅ are each independently selected from the group consisting of hydrogen, halogen, straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl, C₁-C₆-alkyl-SO₂ or N(R)R′, wherein R and R′ are each independently hydrogen, halogen, straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl or C₁-C₆-alkyl-SO₂, provided that at least one of R₁, R₂, R₄ and R₅ is not hydrogen. R⁶ is —OH, —CO₂R⁹, —CH₂═CH(CO)OR⁹, —OPO₂R¹⁰R^{11, —OPO}₃R¹⁰R¹¹, —CH₂PO₃R¹⁰R¹¹, —OPO₂(S)R¹⁰R¹¹ or —C(Y)(X)PO₃R¹⁰R¹¹, where X is hydroxyl or halide and Y is H or halide; or analogues of other carboxylate, phosphate or phosphonate isosteres not limited to those shown below; R⁹ is H, straight chain or branched C₁-C₆-alkyl, or a substituted or unsubstituted aryl group; R¹⁰ and R¹¹ are each independently H, straight chain or branched C₁-C₆-alkyl, a substituted or unsubstituted aryl group or selected from, but not limited to, the prodrugs listed below: 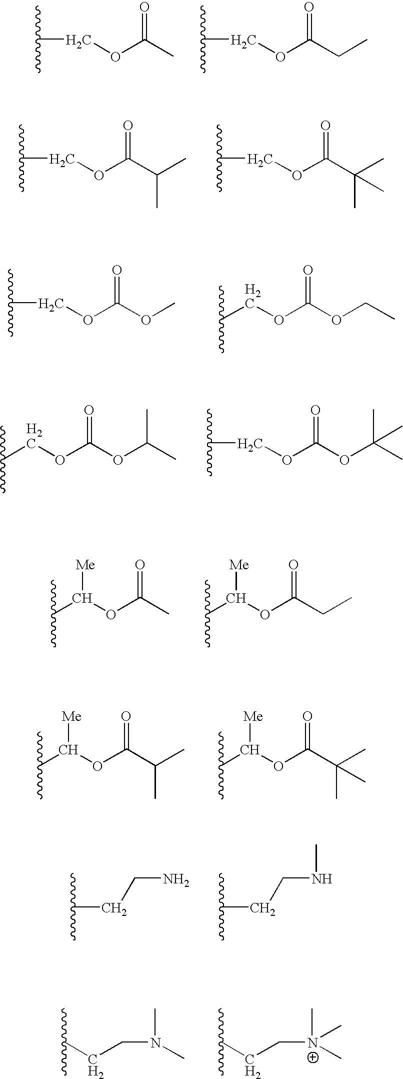
R₇ is H, C₁-C₆-alkyl, hydroxy-C₁-C₆-alkyl or aryl. R₈ is H or C₁-C₆-alkyl. R₇ and R₈ can also together form a C₂-C₅-alkylene or a C₂-C₅-alkenylene group; m and n are each, independently, an integer from 0 to 3; provided that when R⁴ is C₄-C₂₀-alkyl, at least one of R¹, R², R³ and R⁵ is not hydrogen; and when R³ is C₄-C₂₀-alkyl, at least one of R¹, R², R⁴ and R⁵ is not hydrogen; and pharmaceutically acceptable salts thereof.

In a fourth set of compounds of Formula II, R₄ is C₄-C₂₀-alkyl; R₁, R₂, R₃ and R₅ are each independently selected from the group consisting of hydrogen, halogen, straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl, C₁-C₆-alkyl-SO₂ or N(R)R′, wherein R and R′ are each independently hydrogen, straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl or C₁-C₆-alkyl-SO₂, provided that at least one of R₁, R₂, R₃ and R₅ is not hydrogen. R⁶ is —OH, —CO₂R⁹, —CH₂═CH(CO)OR⁹, —OPO₂R¹⁰R¹¹, —OPO₃R¹⁰R¹¹, —CH₂PO₃R¹⁰R¹¹, —OPO₂(S)R¹⁰OR¹¹ or —C(Y)(X)PO₃R¹⁰R¹¹, where X is hydroxyl or halide and Y is H or halide; or analogues of other carboxylate, phosphate or phosphonate isosteres not limited to those shown below; R⁹ is H, straight chain or branched C₁-C₆-alkyl, or a substituted or unsubstituted aryl group R¹⁰ and R¹¹ are each independently H, straight chain or branched C₁-C₆-alkyl, a substituted or unsubstituted aryl group or selected from, but not limited to, the prodrugs listed below: 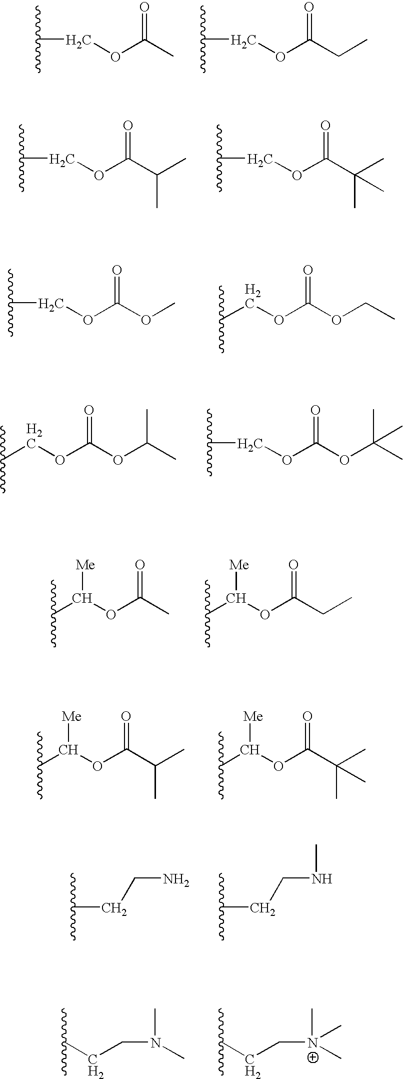
R₇ is H, C₁-C₆-alkyl, hydroxy-C₁-C₆-alkyl or aryl. R₈ is H or C₁-C₆-alkyl. R₇ and R₈ can also together form a C₂-C₅-alkylene or a C₂-C₅-alkenylene group; m and n are each, independently, an integer from 0 to 3; provided that when R⁴ is C₄-C₂₀-alkyl, at least one of R¹, R², R³ and R⁵ is not hydrogen; and when R³ is C₄-C₂₀-alkyl, at least one of R¹, R², R⁴ and R⁵ is not hydrogen; and pharmaceutically acceptable salts thereof;

In certain embodiments, compounds of the invention are the compounds of Formula IV: 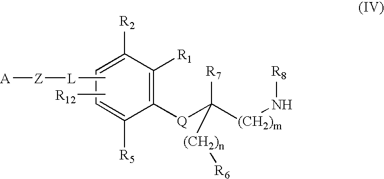
wherein:

L is alkoxy, a covalent bond, substituted or unsubstituted alkyl, alkylcarbonyl, thioether, alkylsulfonyl, alkylcarbonylamino, alkylaminocarbonyl, alkyloxycarbonyl, alkylcarbonyloxy, or substituted or unsubstituted heteroaryl;

Z and A are each independently substituted or unsubstituted aryl, wherein Z and A may be linked by a covalent bond, substituted or unsubstituted alkyl, NH, alkyloxy, O, thioether, S, aminocarbonyl, carbonylamino, carbonyloxy, or oxycarbonyl;

R¹, R², R⁵ and R¹² are each independently selected from the group consisting of hydrogen, halogen, cyano, substituted or unsubstituted aryl, straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl, C₁-C₆-alkyl-SO₂ or N(R)R′, wherein R and R′ are each independently hydrogen, straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl or C₁-C₆-alkyl-SO₂;

Q is —CH₂NR—, —CH₂NR(CO)—, —NH(CO)—, —(CO)NH—, —(CO)—, —O—, —S—, —SO—, —SO₂—, —NRSO₂—, —SO₂—NR— or heteroaryl, where R is hydrogen or straight chain or branched C₁-C₆-alkyl;

R⁶ is —OH, —CO₂R⁹, —CH₂═CH(CO)OR⁹, —OPO₂R¹⁰OR¹¹, —OPO₃R¹⁰R¹¹, —CH₂PO₃R¹⁰R¹¹, —OPO₂(S)R¹⁰OR¹¹ or —C(Y)(X)PO₃R¹⁰R¹¹, where X is hydroxyl or halide and Y is H or halide; or analogues of other carboxylate, phosphate or phosphonate isosteres not limited to those shown below; R⁹ is H, straight chain or branched C₁-C₆-alkyl, or a substituted or unsubstituted aryl group; R¹⁰ and R¹¹ are each independently H, straight chain or branched C₁-C₆-alkyl, a substituted or unsubstituted aryl group or selected from, but not limited to, the prodrugs listed below:

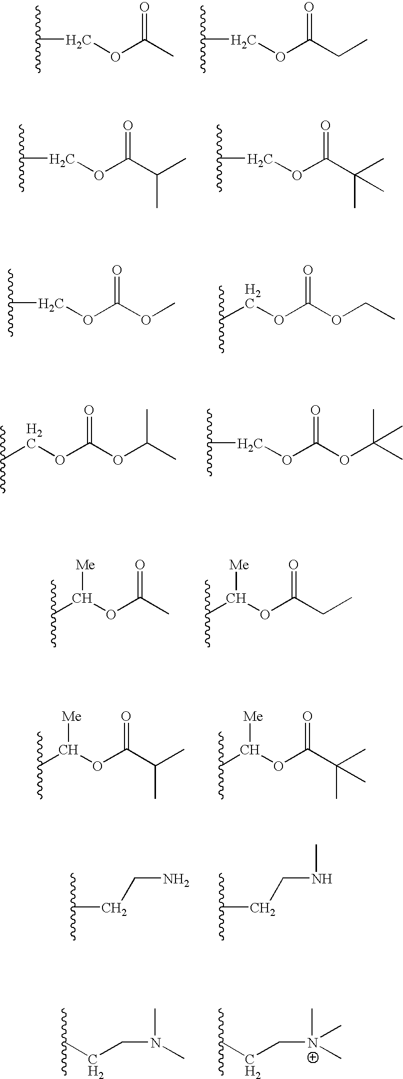

R⁷ is H, C₁-C₆-alkyl, hydroxy-C₁-C₆-alkyl, aryl, or together with R8 form a C₂-C₅-alkylene or a C₂-C₅-alkenylene group;

R⁸ is H or C₁-C₆-alkyl; and

m and n are each, independently, an integer from 0 to 3; provided that when R⁴ is C₄-C₂₀-alkyl, at least one of R¹, R², R³ and R⁵ is not hydrogen; and when R³ is C₄-C₂₀-alkyl, at least one of R¹, R², R⁴ and R⁵ is not hydrogen; and pharmaceutically acceptable salts thereof.

In yet another embodiment, the present invention provides compounds of Formula V:

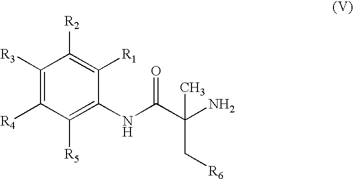

In a first set of compounds of Formula V, R₃ is C₆-C₁₂-alkoxy or an oxaalkyl, thiaalkyl or azaalkyl group having a chain length of from 6 to 12 atoms; a phenyl or C1-C6-alkylphenyl group, a phenoxy or C1-C6-alkylphenoxy group, a substituted or unsubstituted arylalkyl group, a substituted or unsubstituted arylalkoxy group, a substituted or unsubstituted heteroarylalkyl group; or a substituted or unsubstituted heteroarylalkoxy group. R₁, R₂, R₄ and R₅ are each independently selected from the group consisting of hydrogen, halogen, straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl, C₁-C₆-alkyl-SO₂ and N(R)R′, wherein R and R′ are each independently hydrogen, straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl or C₁-C₆-alkyl-SO₂. R⁶ is —OH, —CO₂R⁹, —CH₂═CH(CO)OR⁹, —OPO₂R¹⁰R¹¹, —OPO₃R¹⁰R¹¹, —CH₂PO₃R¹⁰R¹¹, —OPO₂(S)R¹⁰R¹¹ or —C(Y)(X)PO₃R¹⁰R¹¹, where X is hydroxyl or halide and Y is H or halide; or analogues of other carboxylate, phosphate or phosphonate isosteres not limited to those shown below; R⁹ is H, straight chain or branched C₁-C₆-alkyl, or a substituted or unsubstituted aryl group; R¹⁰ and R¹¹ are each independently H, straight chain or branched C₁-C₆-alkyl, a substituted or unsubstituted aryl group or selected from, but not limited to, the prodrugs listed below:

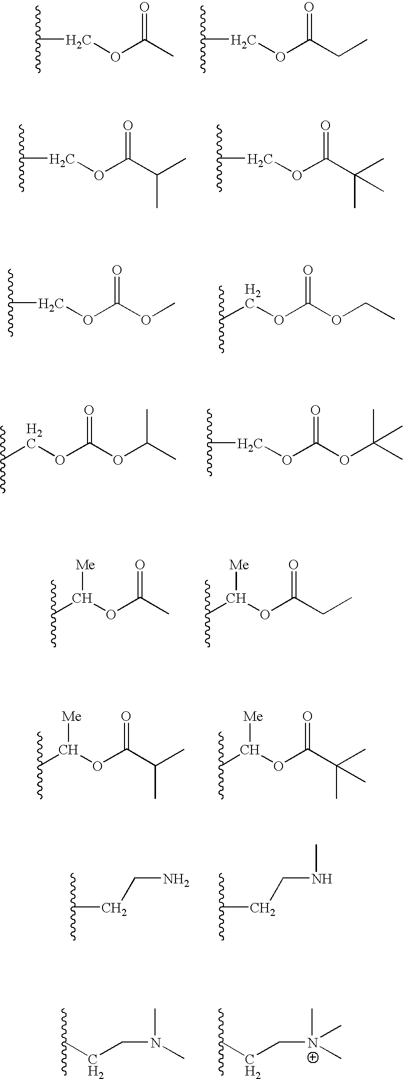

In a second set of compounds of Formula V, R₄ is C₆-C₁₂-alkoxy or an oxaalkyl, thiaalkyl or azaalkyl group having a chain length of from 6 to 12 atoms; a phenyl or C1-C6-aalkylphenyl group, a phenoxy or C1-C6-alkylphenoxy group, a substituted or unsubstituted arylalkyl group, a substituted or unsubstituted arylalkoxy group, a substituted or unsubstituted heteroarylalkyl group; or a substituted or unsubstituted heteroarylalkoxy group. R₁, R₂, R₃ and R₅ are each independently selected from the group consisting of hydrogen, halogen, straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl, C₁-C₆-alkyl-SO₂ or N(R)R′, wherein R and R′ are each independently hydrogen, straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl or C₁-C₆-alkyl-SO₂. R⁶ is —OH, —CO₂R⁹, —CH₂═CH(CO)OR⁹, —OPO₂R¹⁰R¹¹, —OPO₃R¹⁰R¹¹, —CH₂PO₃R¹⁰R¹¹, —OPO₂(S)R¹⁰R¹¹ or —C(Y)(X)PO₃R¹⁰R¹¹, where X is hydroxyl or halide and Y is H or halide; or analogues of other carboxylate, phosphate or phosphonate isosteres not limited to those shown below; R⁹ is H, straight chain or branched C₁-C₆-alkyl, or a substituted or unsubstituted aryl group; R¹⁰ and R¹¹ are each independently H, straight chain or branched C₁-C₆-alkyl, a substituted or unsubstituted aryl group or selected from, but not limited to, the prodrugs listed below:

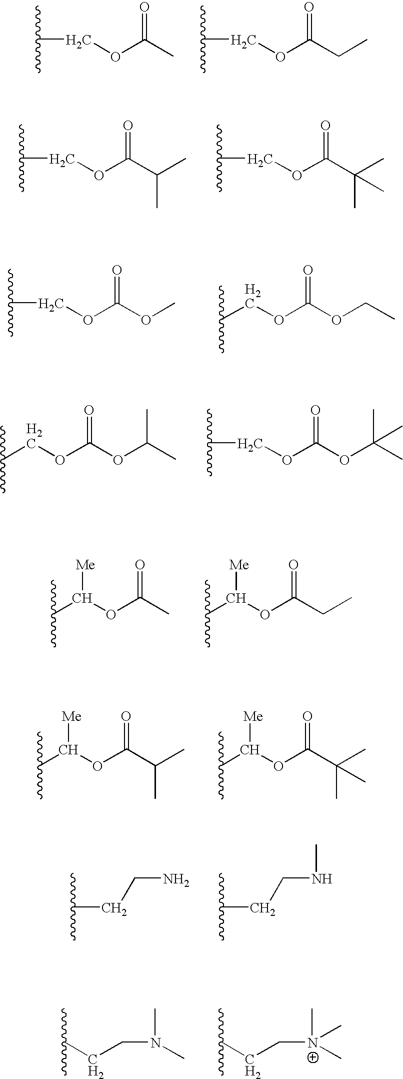

In a third set of compounds of Formula V, R₃ is C₆-C₁₂-alkyl; R₁, R₂, R₄ and R₅ are each independently selected from the group consisting of hydrogen, halogen, straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl, C₁-C₆-alkyl-SO₂ or N(R)R′, wherein R and R′ are each independently hydrogen, straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl or C₁-C₆-alkyl-SO₂, and at least one of R₁, R₂, R₄ and R₅ is not hydrogen. R⁶ is —OH, —CO₂R⁹, —CH₂═CH(CO)OR⁹, —OPO₂R¹⁰R¹¹, —OPO₃R¹⁰R¹¹, —CH₂PO₃R¹⁰R¹¹, —OPO₂(S)R¹⁰R¹¹ or —C(Y)(X)PO₃R¹⁰R¹¹, where X is hydroxyl or halide and Y is H or halide; or analogues of other carboxylate, phosphate or phosphonate isosteres not limited to those shown below; R₉ is H, straight chain or branched C₁-C₆-alkyl, or a substituted or unsubstituted aryl group; R¹⁰ and R¹¹ are each independently H, straight chain or branched C₁-C₆-alkyl, a substituted or unsubstituted aryl group or selected from, but not limited to, the prodrugs listed below: 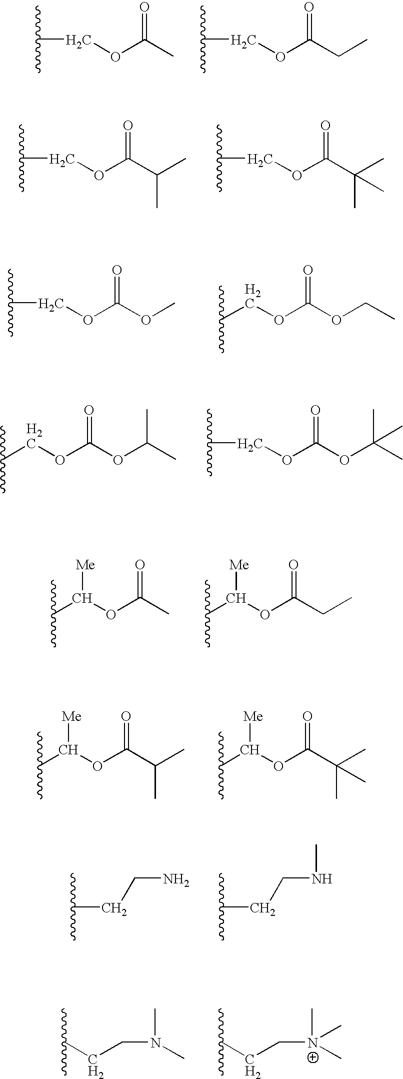
provided that when R⁴ is C₄-C₂₀-alkyl, at least one of R¹, R², R³ and R⁵ is not hydrogen; and when R³ is C₄-C₂₀-alkyl, at least one of R¹, R², R⁴ and R⁵ is not hydrogen; and pharmaceutically acceptable salts thereof.

In a fourth set of compounds of Formula V, R₄ is C₆-C₁₂-alkyl; R₁, R₂, R₃ and R₅ are each independently selected from the group consisting of hydrogen, halogen, straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl, C₁-C₆-alkyl-SO₂ and N(R)R′, wherein R and R′ are each independently hydrogen, straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl or C₁-C₆-alkyl-SO₂, and at least one of R₁, R₂, R₃ and R₅ is not hydrogen. R⁶ is —OH, —CO₂R⁹, —CH₂═CH(CO)OR⁹, —OPO₂R¹⁰R¹¹, —OPO₃R¹⁰R¹¹, —CH₂PO₃R¹⁰R¹¹, —OPO₂(S)R¹⁰R¹¹ or —C(Y)(X)PO₃R¹⁰R¹¹, where X is hydroxyl or halide and Y is H or halide; or analogues of other carboxylate, phosphate or phosphonate isosteres not limited to those shown below; R⁹ is H, straight chain or branched C₁-C₆-alkyl, or a substituted or unsubstituted aryl group; R¹⁰ and R¹¹ are each independently H, straight chain or branched C₁-C₆-alkyl, a substituted or unsubstituted aryl group or selected from, but not limited to, the prodrugs listed in the below:

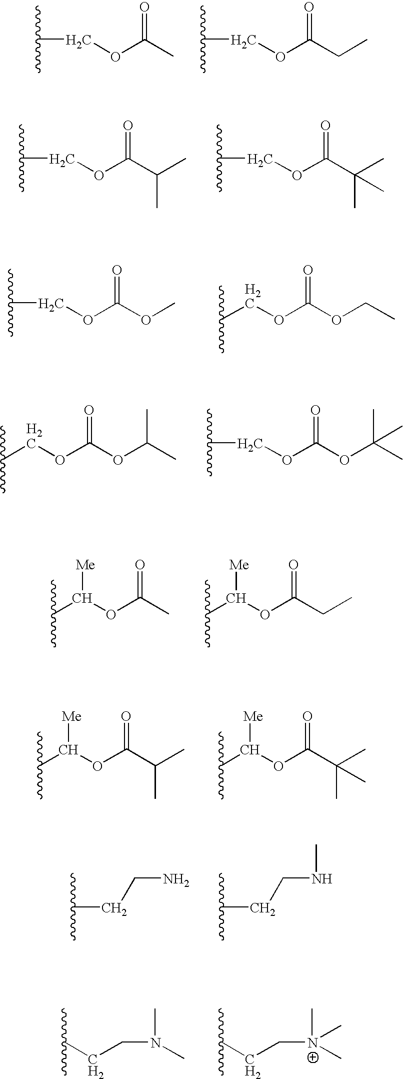

The compounds of Formula I can have the stereochemistry shown below as Formula V or Formula VI, wherein R₁-R₈ have the meanings given above for Formula I:

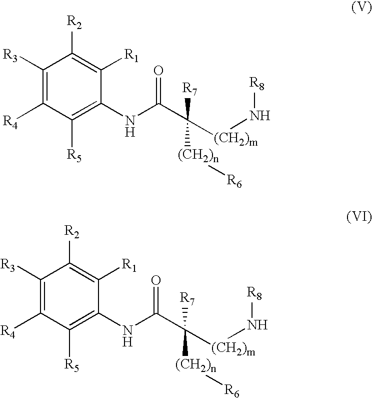

In a first subset of compounds of Formula VI, R₄ is CH₃(CH₂)₇—O— or CH₃(CH₂)₆—O—; and R₁, R₂, R₃ and R₅ are independently selected from the group consisting of hydrogen, methyl, chloro, fluoro, and methoxy. In a particular embodiment, at least one of R₁, R₂, R₃ and R₅ is not hydrogen.

In a second subset of compounds of Formula VI, R₃ is CH₃(CH₂)₇—O— or CH₃(CH₂)₆—O—; and R₁, R₂, R₄ and R₅ are independently selected from the group consisting of hydrogen, methyl, chloro, fluoro, trifluoromethyl and methoxy. In a particular embodiment, at least one of R₁, R₂, R₄ and R₅ is not hydrogen.

In a third subset of compounds of Formula VI, R₄ is CH₃(CH₂)₈— or CH₃(CH₂)₇—; and R₁, R₂, R₃ and R₅ are independently selected from the group consisting of hydrogen, methyl, chloro, fluoro, trifluoromethyl, and methoxy, provided that at least one of R₁, R₂, R₃ and R₅ is not hydrogen.

In a fourth subset of compounds of Formula VI, R₃ is CH₃(CH₂)₈— or CH₃(CH₂)₇—; and R₁, R₂, R₄ and R₅ are independently selected from the group consisting of hydrogen, methyl, chloro, fluoro, trifluoromethyl and methoxy, provided that at least one of R₁, R₂, R₄ and R₅ is not hydrogen.

In a first subset of compounds of Formula VII, R₄ is CH₃(CH₂)₇—O— or CH₃(CH₂)₆—O—; and R₁, R₂, R₃ and R₅ are independently selected from the group consisting of hydrogen, methyl, chloro, fluoro, and methoxy. In a preferred embodiment, at least one of R₁, R₂, R₃ and R₅ is not hydrogen.

In a second subset of compounds of Formula VII, R₃ is CH₃(CH₂)₇—O— or CH₃(CH₂)₆—O—; and R1, R₂, R₄ and R₅ are independently selected from the group consisting of hydrogen, methyl, chloro, fluoro, trifluoromethyl and methoxy. In a preferred embodiment, at least one of R₁, R₂, R₄ and R₅ is not hydrogen.

In a third subset of compounds of Formula VIII, R₄ is CH₃(CH₂)₈— or CH₃(CH₂)₇—; and R₁, R₂, R₃ and R₅ are independently selected from the group consisting of hydrogen, methyl, chloro, fluoro, trifluoromethyl, and methoxy, provided that at least one of R₁, R₂, R₃ and R₅ is not hydrogen.

In a fourth subset of compounds of Formula VIII, R₃ is CH₃(CH₂)₈— or CH₃(CH₂)₇—; and R₁, R₂, R₄ and R₅ are independently selected from the group consisting of hydrogen, methyl, chloro, fluoro, trifluoromethyl and methoxy, provided that at least one of R₁, R₂, R₄ and R₅ is not hydrogen.

A particular subset of compounds of Formula III includes compounds of Formula IX: 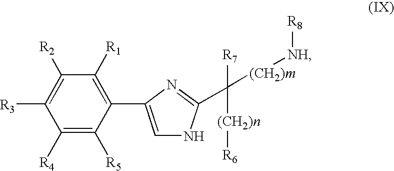
wherein:

one of R₃ and R₄ is selected from the group consisting of C₆-C₁₂-alkoxy or an oxaalkyl, thiaalkyl or azaalkyl group having a chain length of from 6 to 12 atoms; a phenyl or C₁-C₆-alkylphenyl group, a phenoxy or C₁-C₆-alkylphenoxy group, a substituted or unsubstituted arylalkyl group, a substituted or unsubstituted arylalkoxy group, a substituted or unsubstituted heteroarylalkyl group; or a substituted or unsubstituted heteroarylalkoxy group;

R₁, R₂, and R₅ are each independently selected from the group consisting of hydrogen, halogen, straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl, C₁-C₆-alkyl-SO₂ and N(R)R′, wherein R and R′ are each independently hydrogen, straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl or C₁-C₆-alkyl-SO₂;

R⁶ is —OH, —CO₂R⁹, —CH₂═CH(CO)OR⁹, —OPO₂R¹⁰R¹¹, —OPO₃R¹⁰R¹¹, —CH₂PO₃R¹⁰R¹¹, —OPO₂(S)R¹⁰R¹¹ or —C(Y)(X)PO₃R¹⁰R¹¹, where X is hydroxyl or halide and Y is H or halide; or analogues of other carboxylate, phosphate or phosphonate isosteres not limited to those shown below; R⁹ is H, straight chain or branched C₁-C₆-alkyl, or a substituted or unsubstituted aryl group; R¹⁰ and R¹¹ are each independently H, straight chain or branched C₁-C₆-alkyl, a substituted or unsubstituted aryl group or selected from, but not limited to, the prodrugs listed below: 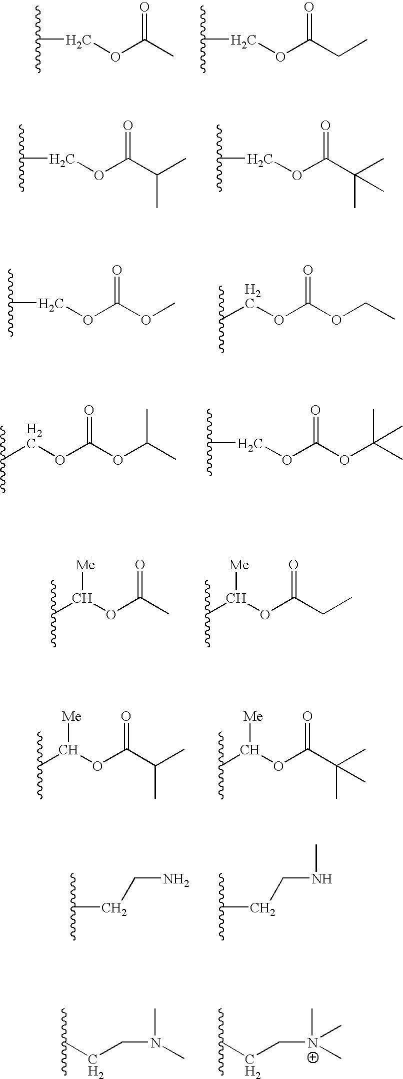
provided that when R⁴ is C₄-C₂₀-alkyl, at least one of R¹, R², R³ and R⁵ is not hydrogen; and when R³ is C₄-C₂₀-alkyl, at least one of R¹, R², R⁴ and R⁵ is not hydrogen; and pharmaceutically acceptable salts thereof.

The invention also provides compounds of Formula X or Formula XI: 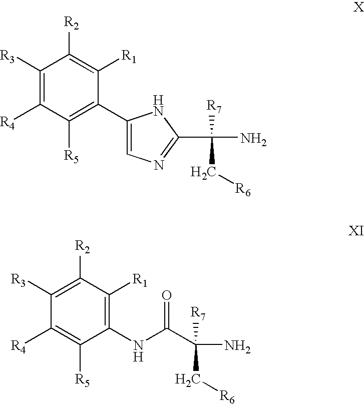
wherein:

one of R₃ and R₄ is selected from the group consisting of optionally substituted C₆-C₁₀-alkoxy, optionally substituted aryl-C₁-C₆-alkoxy, optionally substituted heteroaryl-C₁-C₆-alkoxy, optionally substituted cycloalkyl-C₁-C₆-alkoxy, optionally substituted aryl-C₁-C₆-alkyl, optionally substituted heteroaryl-C₁-C₆-alkyl, optionally substituted cycloalkyl-C₁-C₆-alkyl, optionally substituted aryl, optionally substituted heteroaryl, optionally substituted aryloxy and optionally substituted heteroaryloxy;

R₁, R₂, and R₅ are each independently selected from the group consisting of halogen, trifluoromethyl, C₁-C₆-alkyl, and C₁-C₆-alkoxy;

R₇ is a C₁-C₆-alkyl group, e.g., methyl; and R⁶ is —OH, —CO₂R⁹, —CH₂═CH(CO)OR⁹, —OPO₂R¹⁰R¹¹, —OPO₃R¹⁰R¹¹, —CH₂PO₃R¹⁰R¹¹, —OPO₂(S)R¹⁰R¹¹ or —C(Y)(X)PO₃R¹⁰R¹¹, where X is hydroxyl or halide and Y is H or halide; or analogues of other carboxylate, phosphate or phosphonate isosteres not limited to those shown below; R⁹ is H, straight chain or branched C₁-C₆-alkyl, or a substituted or unsubstituted aryl group; R¹⁰ and R¹¹ are each independently H, straight chain or branched C₁-C₆-alkyl, a substituted or unsubstituted aryl group or selected from, but not limited to, the prodrugs listed below: 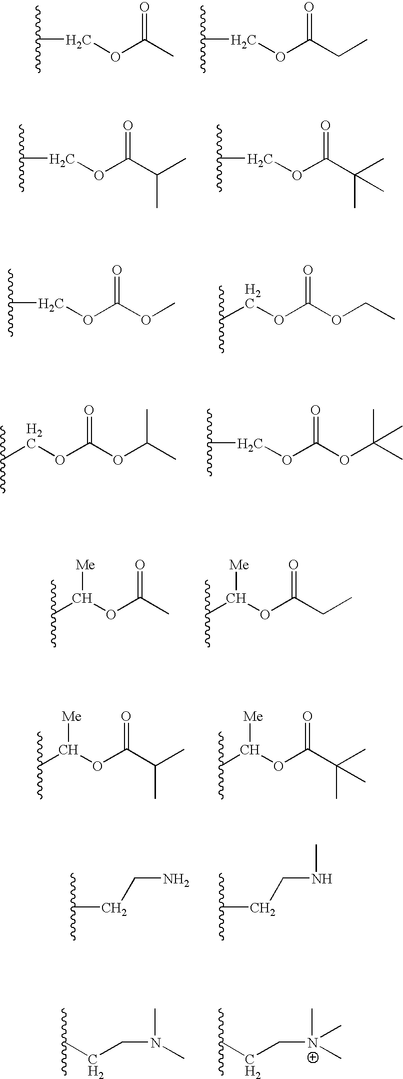
and pharmaceutically acceptable salts, esters and prodrugs thereof.

In certain embodiments, one of R₃ and R₄ is biphenyl-C₁-C₄-alkoxy, where the biphenyl group optionally includes one or more substituents selected from C₁-C₄-alkyl, C₁-C₄-alkenyl, C₁-C₄-alkoxy, cyano, halogen and trifluoromethyl; phenyl-C₁-C₄-alkoxy, wherein the phenyl group optionally includes one or more substituents selected from C₁-C₄-alkyl, C₁-C₄-alkenyl, C₁-C₄-alkoxy, cyano, halogen, methylenedioxy, and trifluoromethyl; naphthyl-C₁-C₄-alkoxy, wherein the naphthyl group optionally includes one or more substituents selected from C₁-C₄-alkyl, C₁-C₄-alkenyl, C₁-C₄-alkoxy, cyano, halogen and trifluoromethyl; C₅-C₈-cycloalkyl-C₁-C₄-alkoxy; heteroaryl-C₁-C₄-alkoxy, wherein the heteroaryl group is imidazolyl; 2-, 3- or 4-pyridyl; or thiophene, optionally substituted by one or more C₁-C₄-alkyl, C₁-C₄-alkenyl, C₁-C₄-alkoxy, cyano, halogen, benzyl, benzyloxy or trifluoromethyl groups; phenyl, optionally substituted by one or more C₁-C₄-alkyl, C₁-C₄-alkenyl, C₁-C₄-alkoxy, cyano, halogen, methylenedioxy, benzyl, benzyloxy or trifluoromethyl groups; naphthyl, optionally substituted by one or more C₁-C₄-alkyl, C₁-C₄-alkenyl, C₁-C₄-alkoxy, cyano, halogen, methylenedioxy, benzyl, benzyloxy or trifluoromethyl groups; or heteroaryl, such as imidazolyl; 2-,3- or 4-pyridyl or thiophene; optionally substituted by one or more C₁-C₄-alkyl, C₁-C₄-alkenyl, C₁-C₄-alkoxy, cyano, halogen, benzyl, benzyloxy or trifluoromethyl groups.

In one set of compounds of Formulae X and XI, R₃ or R₄ is a group selected from, but not limited to, those shown below:

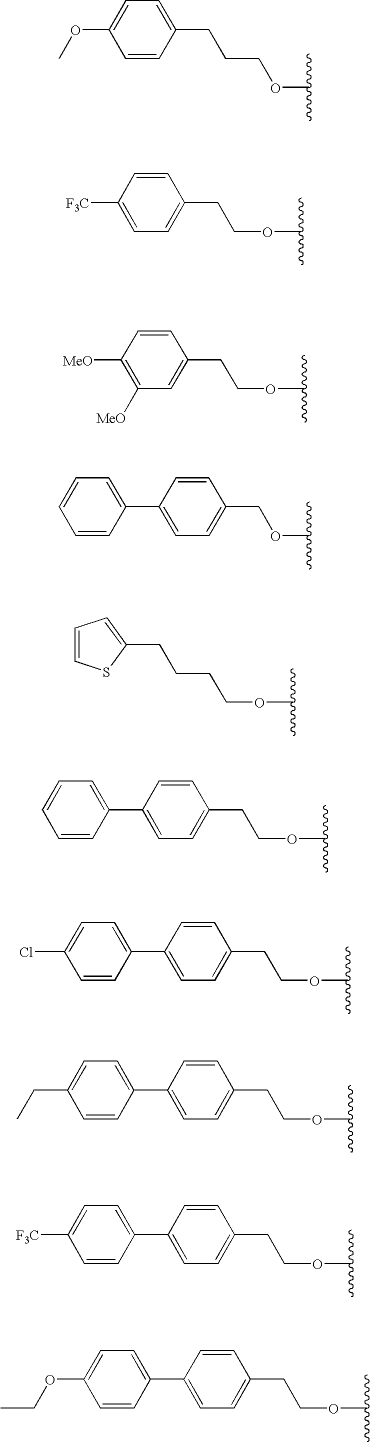

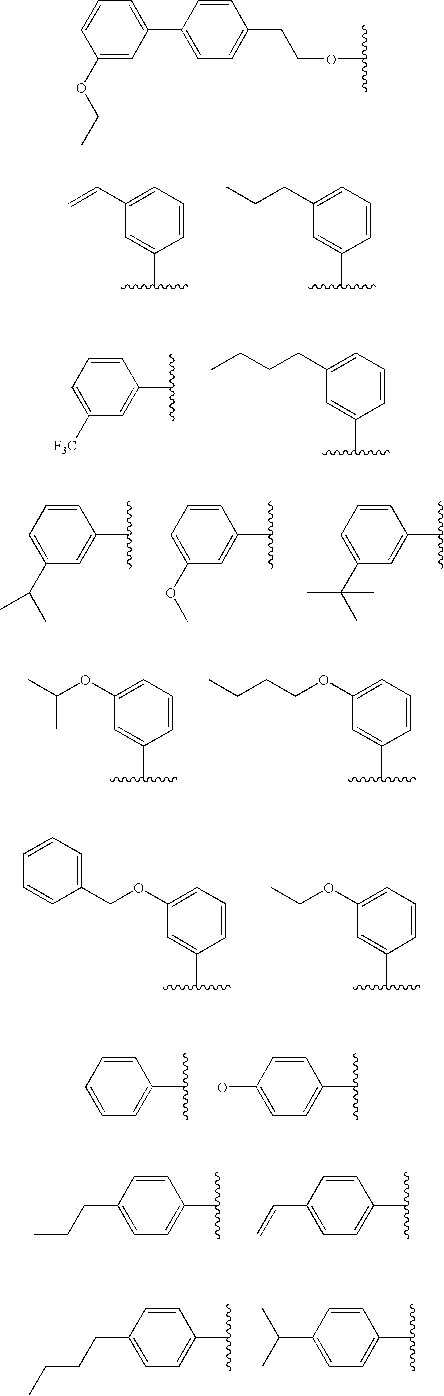

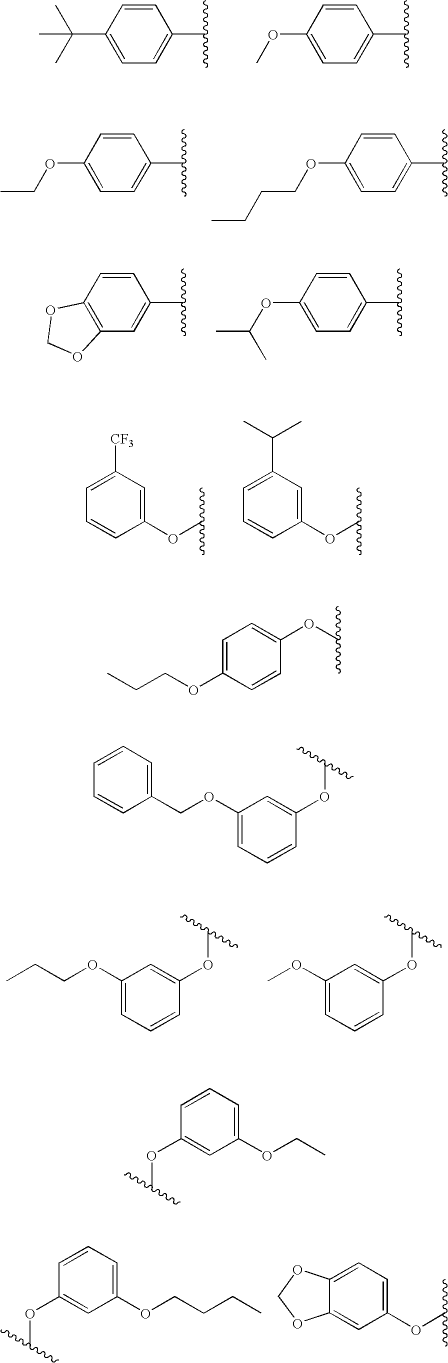

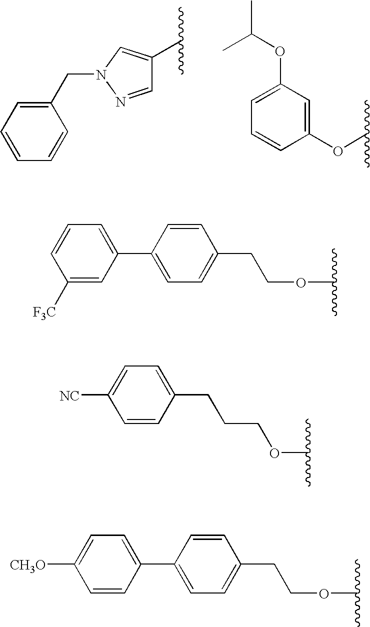

The invention also provides compounds of Formula XA: 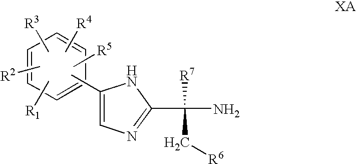
wherein:

R₃ is selected from the group consisting of optionally substituted C₆-C₁₀-alkoxy, optionally substituted aryl-C₁-C₆-alkoxy, optionally substituted heteroaryl-C₁-C₆-alkoxy, optionally substituted cycloalkyl-C₁-C₆-alkoxy, optionally substituted aryl-C₁-C₆-alkyl, optionally substituted heteroaryl-C₁-C₆-alkyl, optionally substituted cycloalkyl-C₁-C₆-alkyl, optionally substituted aryl, optionally substituted heteroaryl, optionally substituted aryloxy and optionally substituted heteroaryloxy;

R₄ is selected from the group consisting of halo-alkyl, e.g., trifluoromethyl, C₁-C₆-alkyl, and C₁-C₆-alkoxy;

R₁, R₂, and R₅ are each independently selected from the group consisting of hydrogen, halogen, trifluoromethyl, C₁-C₆-alkyl, and C₁-C₆-alkoxy;

R₇ is a C₁-C₆-alkyl group, e.g., methyl; and R⁶ is —OH, —CO₂R⁹, —CH₂═CH(CO)OR⁹, —OPO₂R¹⁰R¹¹, —OPO₃R¹⁰R¹¹, —CH₂PO₃R¹⁰R¹¹, —OPO₂(S)R¹⁰R¹¹ or —C(Y)(X)PO₃R¹⁰R¹¹, where X is hydroxyl or halide and Y is H or halide; or analogues of other carboxylate, phosphate or phosphonate isosteres not limited to those shown below; R⁹ is H, straight chain or branched C₁-C₆-alkyl, or a substituted or unsubstituted aryl group; R¹⁰ and R¹¹ are each independently H, straight chain or branched C₁-C₆-alkyl, a substituted or unsubstituted aryl group or selected from, but not limited to, the prodrugs listed below: 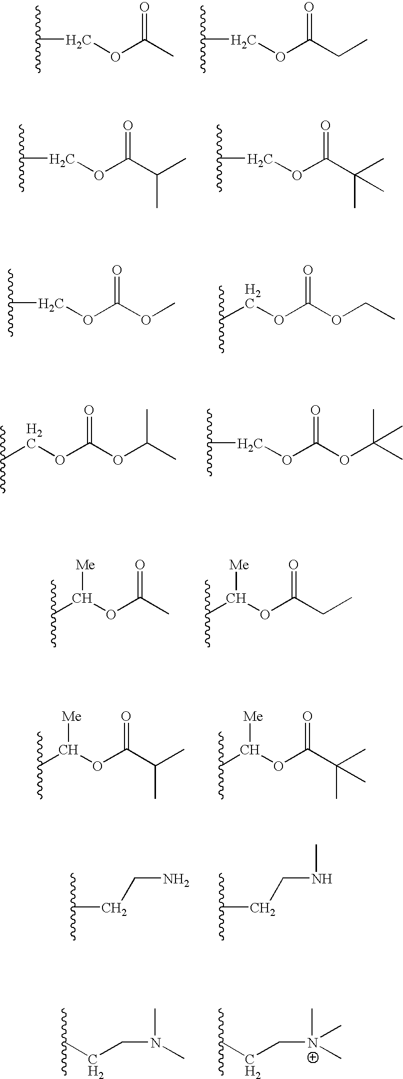
and pharmaceutically acceptable salts, esters and prodrugs thereof.

In certain embodiments, R₃ is biphenyl-C₁-C₄-alkoxy, where the biphenyl group optionally includes one or more substituents selected from C₁-C₄-alkyl, C₁-C₄-alkenyl, C₁-C₄-alkoxy, cyano, halogen and trifluoromethyl; phenyl-C₁-C₄-alkoxy, wherein the phenyl group optionally includes one or more substituents selected from C₁-C₄-alkyl, C₁-C₄-alkenyl, C₁-C₄-alkoxy, cyano, halogen, methylenedioxy, and trifluoromethyl; naphthyl-C₁-C₄-alkoxy, wherein the naphthyl group optionally includes one or more substituents selected from C₁-C₄-alkyl, C₁-C₄-alkenyl, C₁-C₄-alkoxy, cyano, halogen and trifluoromethyl; C₅-C₈-cycloalkyl-C₁-C₄-alkoxy; heteroaryl-C₁-C₄-alkoxy, wherein the heteroaryl group is imidazolyl; 2-, 3- or 4-pyridyl; or thiophene, optionally substituted by one or more C₁-C₄-alkyl, C₁-C₄-alkenyl, C₁-C₄-alkoxy, cyano, halogen, benzyl, benzyloxy or trifluoromethyl groups; phenyl, optionally substituted by one or more C₁-C₄-alkyl, C₁-C₄-alkenyl, C₁-C₄-alkoxy, cyano, halogen, methylenedioxy, benzyl, benzyloxy or trifluoromethyl groups; naphthyl, optionally substituted by one or more C₁-C₄-alkyl, C₁-C₄-alkenyl, C₁-C₄-alkoxy, cyano, halogen, methylenedioxy, benzyl, benzyloxy or trifluoromethyl groups; or heteroaryl, such as imidazolyl; 2-,3- or 4-pyridyl or thiophene; optionally substituted by one or more C₁-C₄-alkyl, C₁-C₄-alkenyl, C₁-C₄-alkoxy, cyano, halogen, benzyl, benzyloxy or trifluoromethyl groups.

The invention also provides compounds of Formula XIA: 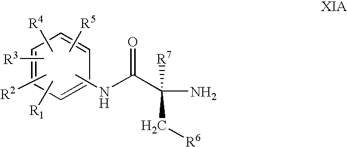
wherein:

R₃ selected from the group consisting of optionally substituted C₆-C₁₀-alkoxy, optionally substituted aryl-C₁-C₆-alkoxy, optionally substituted heteroaryl-C₁-C₆-alkoxy, optionally substituted cycloalkyl-C₁-C₆-alkoxy, optionally substituted aryl-C₁-C₆-alkyl, optionally substituted heteroaryl-C₁-C₆-alkyl, optionally substituted cycloalkyl-C₁-C₆-alkyl, optionally substituted aryl, optionally substituted heteroaryl, optionally substituted aryloxy and optionally substituted heteroaryloxy;

R₄ is selected from the group consisting of halo-alkyl, e.g., trifluoromethyl;

R₁, R₂, and R₅ are each independently selected from the group consisting of hydrogen, halogen, trifluoromethyl, C₁-C₆-alkyl, and C₁-C₆-alkoxy;

R₇ is a C₁-C₆-alkyl group, e.g., methyl; and R⁶ is —OH, —CO₂R⁹, —CH₂═CH(CO)OR⁹, —OPO₂R¹⁰ R¹¹, —OPO₃R¹⁰R¹¹, —CH₂PO₃R¹⁰R¹¹, —OPO₂(S)R¹⁰R¹¹ or —C(Y)(X)PO₃R¹⁰R¹¹, where X is hydroxyl or halide and Y is H or halide; or analogues of other carboxylate, phosphate or phosphonate isosteres not limited to those shown below; R⁹ is H, straight chain or branched C₁-C₆-alkyl, or a substituted or unsubstituted aryl group; R¹⁰ and R¹¹ are each independently H, straight chain or branched C₁-C₆-alkyl, a substituted or unsubstituted aryl group or selected from, but not limited to, the prodrugs listed below: 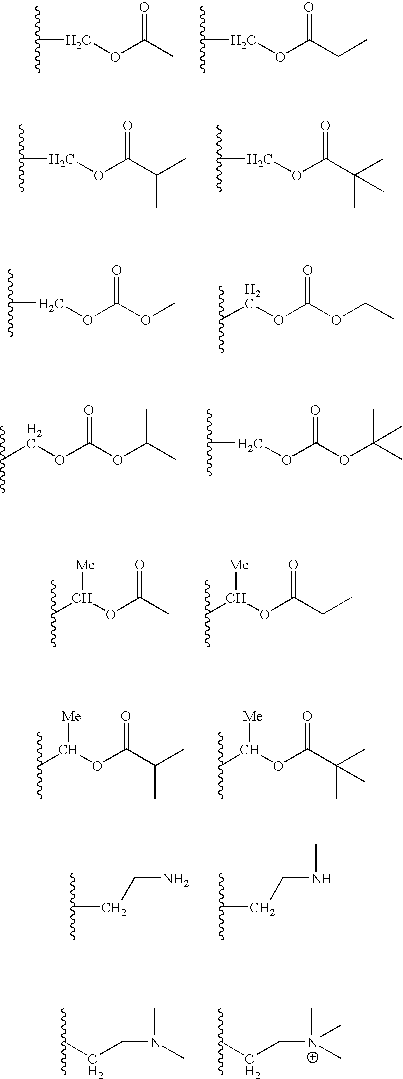
and pharmaceutically acceptable salts, esters and prodrugs thereof.

In certain embodiments, R₃ is biphenyl-C₁-C₄-alkoxy, where the biphenyl group optionally includes one or more substituents selected from C₁-C₄-alkyl, C₁-C₄-alkenyl, C₁-C₄-alkoxy, cyano, halogen and trifluoromethyl; phenyl-C₁-C₄-alkoxy, wherein the phenyl group optionally includes one or more substituents selected from C₁-C₄-alkyl, C₁-C₄-alkenyl, C₁-C₄-alkoxy, cyano, halogen, methylenedioxy, and trifluoromethyl; naphthyl-C₁-C₄-alkoxy, wherein the naphthyl group optionally includes one or more substituents selected from C₁-C₄-alkyl, C₁-C₄-alkenyl, C₁-C₄-alkoxy, cyano, halogen and trifluoromethyl; C₅-C₈-cycloalkyl-C₁-C₄-alkoxy; heteroaryl-C₁-C₄-alkoxy, wherein the heteroaryl group is imidazolyl; 2-, 3- or 4-pyridyl; or thiophene, optionally substituted by one or more C₁-C₄-alkyl, C₁-C₄-alkenyl, C₁-C₄-alkoxy, cyano, halogen, benzyl, benzyloxy or trifluoromethyl groups; phenyl, optionally substituted by one or more C₁-C₄-alkyl, C₁-C₄-alkenyl, C₁-C₄-alkoxy, cyano, halogen, methylenedioxy, benzyl, benzyloxy or trifluoromethyl groups; naphthyl, optionally substituted by one or more C₁-C₄-alkyl, C₁-C₄-alkenyl, C₁-C₄-alkoxy, cyano, halogen, methylenedioxy, benzyl, benzyloxy or trifluoromethyl groups; or heteroaryl, such as imidazolyl; 2-,3- or 4-pyridyl or thiophene; optionally substituted by one or more C₁-C₄-alkyl, C₁-C₄-alkenyl, C₁-C₄-alkoxy, cyano, halogen, benzyl, benzyloxy or trifluoromethyl groups.

In another embodiment, the invention is directed to a compound of the following formulae: 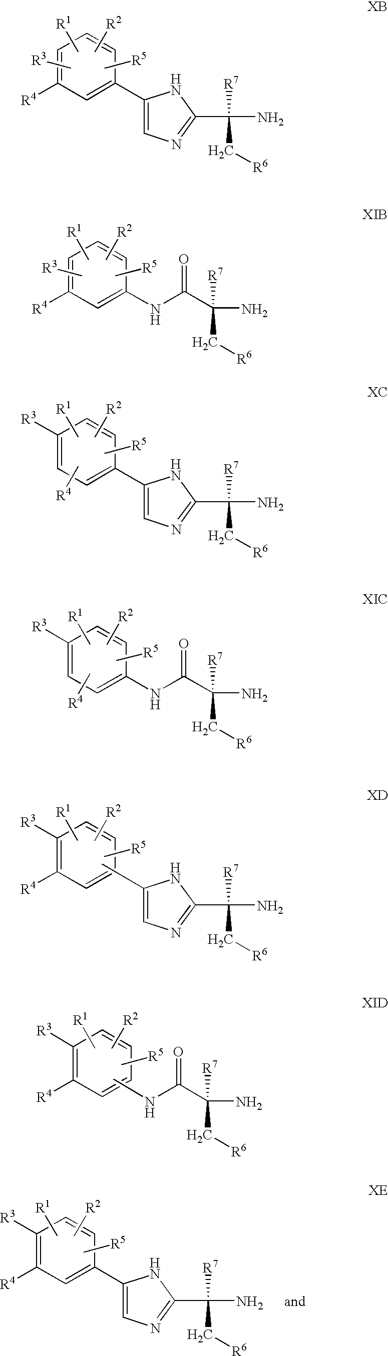 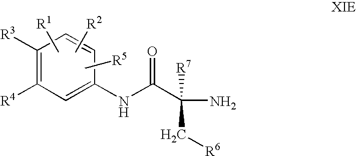
wherein:

R₃ selected from the group consisting of optionally substituted C₆-C₁₀-alkoxy, optionally substituted aryl-C₁-C₆-alkoxy, optionally substituted heteroaryl-C₁-C₆-alkoxy, optionally substituted cycloalkyl-C₁-C₆-alkoxy, optionally substituted aryl-C₁-C₆-alkyl, optionally substituted heteroaryl-C₁-C₆-alkyl, optionally substituted cycloalkyl-C₁-C₆-alkyl, optionally substituted aryl, optionally substituted heteroaryl, optionally substituted aryloxy and optionally substituted heteroaryloxy;

R₄ is selected from the group consisting of halogen, halo-alkyl, e.g., trifluoromethyl, C₁-C₆-alkyl, and C₁ -C₆-alkoxy;

R₁, R₂, and R₅ are each independently selected from the group consisting of hydrogen, halogen, trifluoromethyl, C₁-C₆-alkyl, and C₁-C₆-alkoxy;

R₇ is a C₁-C₆-alkyl group, e.g., methyl; and R⁶ is —OH, —CO₂R⁹, —CH₂═CH(CO)OR⁹, —OPO₂R¹⁰R¹¹, —OPO₃R¹⁰R¹¹, —CH₂PO₃R¹⁰R¹¹, —OPO₂(S)R¹⁰R¹¹ or —C(Y)(X)PO₃R¹⁰R¹¹, where X is hydroxyl or halide and Y is H or halide; or analogues of other carboxylate, phosphate or phosphonate isosteres not limited to those shown below; R⁹ is H, straight chain or branched C₁-C₆-alkyl, or a substituted or unsubstituted aryl group; R¹⁰ and R¹¹ are each independently H, straight chain or branched C₁-C₆-alkyl, a substituted or unsubstituted aryl group or selected from, but not limited to, the prodrugs listed below: 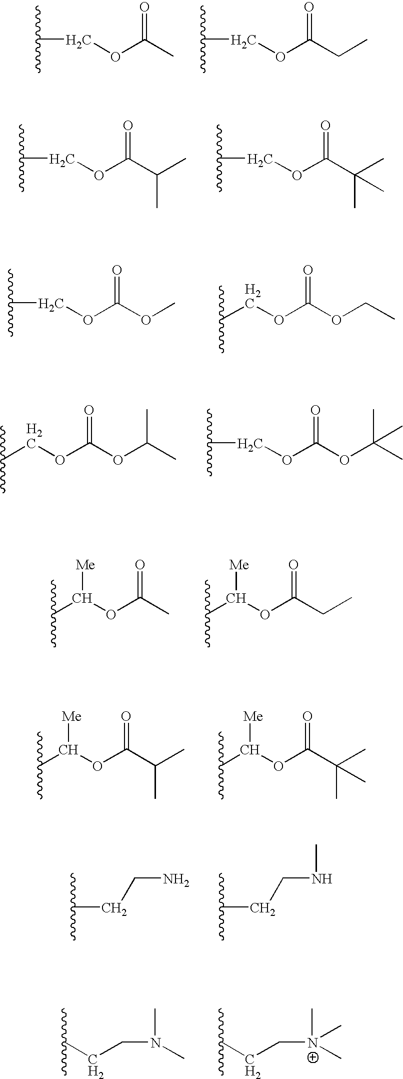
and pharmaceutically acceptable salts, esters and prodrugs thereof.

In certain embodiments, R₃ is biphenyl-C₁-C₄-alkoxy, where the biphenyl group optionally includes one or more substituents selected from C₁-C₄-alkyl, C₁-C₄-alkenyl, C₁-C₄-alkoxy, cyano, halogen and trifluoromethyl; phenyl-C₁-C₄-alkoxy, wherein the phenyl group optionally includes one or more substituents selected from C₁-C₄-alkyl, C₁-C₄-alkenyl, C₁-C₄-alkoxy, cyano, halogen, methylenedioxy, and trifluoromethyl; naphthyl-C₁-C₄-alkoxy, wherein the naphthyl group optionally includes one or more substituents selected from C₁-C₄-alkyl, C₁-C₄-alkenyl, C₁-C₄-alkoxy, cyano, halogen and trifluoromethyl; C₅-C₈-cycloalkyl-C₁-C₄-alkoxy; heteroaryl-C₁-C₄-alkoxy, wherein the heteroaryl group is imidazolyl; 2-, 3- or 4-pyridyl; or thiophene, optionally substituted by one or more C₁-C₄-alkyl, C₁-C₄-alkenyl, C₁-C₄-alkoxy, cyano, halogen, benzyl, benzyloxy or trifluoromethyl groups; phenyl, optionally substituted by one or more C₁-C₄-alkyl, C₁-C₄-alkenyl, C₁-C₄-alkoxy, cyano, halogen, methylenedioxy, benzyl, benzyloxy or trifluoromethyl groups; naphthyl, optionally substituted by one or more C₁-C₄-alkyl, C₁-C₄-alkenyl, C₁-C₄-alkoxy, cyano, halogen, methylenedioxy, benzyl, benzyloxy or trifluoromethyl groups; or heteroaryl, such as imidazolyl; 2-,3- or 4-pyridyl or thiophene; optionally substituted by one or more C₁-C₄-alkyl, C₁-C₄-alkenyl, C₁-C₄-alkoxy, cyano, halogen, benzyl, benzyloxy or trifluoromethyl groups. In certain embodiments, R₄ is selected from the group consisting of halo-alkyl, e.g., trifluoromethyl, C₁-C₆-alkyl, and C₁-C₆-alkoxy. In certain embodiments, R₄ is selected from the group consisting of halo-alkyl, e.g., trifluoromethyl.

In another embodiment, the invention is directed to a compound of Formula XII: 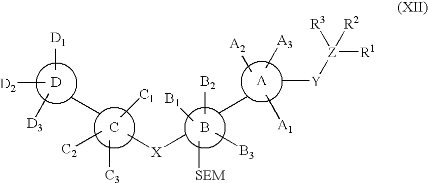
wherein:

SEM represents a selectivity enhancing moiety;

rings A, B, C, D are independently selected from the group consisting of substituted or unsubstituted carbocyclic rings and substituted or unsubstituted heterocyclic rings, which may contain one or more heteroatoms and may be saturated or unsaturated;

A₁, A₂, A₃, B₁, B₂, B₃, C₁, C₂, C₃, D₁, D₂, and D₃ are each independently selected from the group consisting of hydrogen, halogen, cyano, straight chain or branched aliphatic, straight chain or branched alkoxy, straight chain or branched halo-alkyl, straight chain or branched halo-alkoxy, alkoxy-alkyl, hydroxyl-alkyl, carboxy-alkyl, alkyl-SO₂ alkylcarbonyl, thioether, alkylsulfonyl, alkylcarbonylamino, alkylaminocarbonyl, alkyloxycarbonyl, alkylcarbonyloxy, substituted or unsubstituted aryl, substituted or unsubstituted heteroaryl, —OH, —C(O)-alkyl, —C(O)-halo-alkyl, —C(O)O-alkyl, —C(O)O-halo-alkyl, —CONH₂, —CONH-alkyl, —CON-dialkyl, —CONH-halo-alkyl, —CON-halo-dialkyl, -alkyl-CONH-alkyl, -alkyl-CON-dialkyl, halo-alkyl-CONH-alkyl, halo-alkyl-CONH-halo-alkyl, alkyl-CONH-halo-dialkyl, -alkyl-hydroxyl, -alkyl-hydroxyl-alkyl, -halo-alkyl-hydroxyl-alkyl, -alkyl-hydroxyl-halo-alkyl, -halo-alkyl-hydroxyl-halo-alkyl, substituted or unsubstituted alkyl-OR¹⁴, substituted or unsubstituted haloalkyl-OR¹⁴, —OR¹⁴, and N(R)R′; or taken together A₃ and B₃ may form a substituted or unsubstituted carbocyclic ring or substituted or unsubstituted heterocyclic ring, which may contain one or more heteroatoms and may be saturated or unsaturated; or taken together C₂ and D₂ may form a substituted or unsubstituted carbocyclic ring or substituted or unsubstituted heterocyclic ring, which may contain one or more heteroatoms and may be saturated or unsaturated;

R and R′ are each independently selected from the group consisting of hydrogen, cyano, straight chain or branched alkyl, straight chain or branched alkoxy, straight chain or branched halo-alkyl, straight chain or branched halo-alkoxy, alkoxy-alkyl, hydroxyl-alkyl, and carboxy-alkyl; or taken together R and R′ may form a substituted or unsubstituted carbocyclic ring or substituted or unsubstituted or heterocyclic ring, which may contain one or more heteroatoms and may be saturated or unsaturated; or taken together with the N to which they are attached R and R′ may form a moiety selected from the group consisting of substituted straight chain or cyclic guanyl, straight chain or cyclic guanidine, straight chain or cyclic urea, straight chain or cyclic thiourea, straight chain or cyclic carbamate, and straight chain or cyclic thiocarbamate;

Z is independently selected from the group consisting of C or N;

R¹ is a phosphate derivative, a phosphate mimic or a phosphate precursor;

R² and R³ are each independently selected from the group consisting of hydrogen, hydroxyl, halogen, cyano, straight chain or branched alkyl, alkyl-OR⁹, halo-alkyl-OR⁹, alkoxy-OR⁹, alkyl-OC(O)R⁹, halo-alkyl-OC(O)R⁹, alkoxy-OC(O)R⁹, carbocyclic rings, heterocyclic rings which may contain one or more heteroatoms, alkyl-NR⁹R¹⁰, halo-alkyl-NR⁹R¹⁰, and alkoxy-NR⁹R¹⁰, all of which may be optionally substituted with OH, halogen, NHR⁹, NR⁹R¹⁰, straight chain or branched alkoxy, straight chain or branched halo-alkyl, straight chain or branched halo-alkoxy, alkoxy-alkyl, hydroxyl-alkyl, or carboxy-alkyl; or taken together R² and R³ may form a substituted or unsubstituted carbocyclic ring or a substituted or unsubstituted heterocyclic ring, which may contain one or more heteroatoms and may be saturated or unsaturated; or taken together R² and A₁ may form a substituted or unsubstituted C₄-C₁₀ fused carbocyclic ring or substituted or unsubstituted C₄-C₁₀ fused heterocyclic rings, which may contain one or more heteroatoms and may be saturated or unsaturated;

R⁹ and R¹⁰ are independently selected from the group consisting of hydrogen, halogen, cyano, straight chain or branched alkyl, straight chain or branched alkoxy, straight chain or branched halo-alkyl, straight chain and branched halo-alkoxy, —C(O)alkyl, —C(O)NH-alkyl, —C(O)N-dialkyl, —C(O)aryl, —C(O)NH-aryl, —C(O)N-alkyl-aryl, —C(O)N-diaryl, —C(O)heteroaryl, —C(O)NH-heteroaryl, —C(O)N-carbocycle, substituted or unsubstituted carbocyclic rings, and substituted or unsubstituted heterocyclic rings, which may contain one or more heteroatoms and may be saturated or unsaturated; or taken together R⁹ and R¹⁰ may form a substituted or unsubstituted carbocyclic ring or a substituted or unsubstituted heterocyclic ring, which may contain one or more heteroatoms and may be saturated or unsaturated; or R⁹ or R¹⁰ together with A₁ may form a substituted or unsubstituted C₄-C₁₀ fused carbocyclic ring or substituted or unsubstituted C₄-C₁₀ fused heterocyclic rings, which may contain one or more heteroatoms and may be saturated or unsaturated;

Y is independently selected from the group consisting of (CR¹¹R¹²)ₙ and (CR¹¹R¹²)ₙNR¹³;

R¹¹, R¹², and R¹³ are independently selected from the group consisting of hydrogen, halogen, cyano, and straight chain or branched alkyl, all of which may be optionally substituted with OH, halogen, straight chain or branched alkoxy, straight chain or branched halo-alkyl, and straight chain and branched halo-alkoxy; or R¹³ may form a 3-8-membered ring together with either R¹¹ or R¹² and the atom to which they are attached;

n is an integer from 0 to 3;

X is selected from the group consisting of

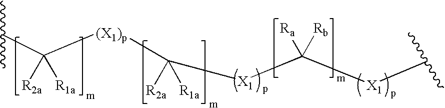

wherein each m is independently selected from an integer between 0 and 6; each p is independently selected from 0 or 1; each X₁ is independently selected from the group consisting of CR¹⁴R¹⁵, NR¹⁴, S, and O, —S(O), —S(O)₂, —OS(O)₂, —OS(O)₂O—, —C(O), C(OH), —C(O)O—, a substituted or unsubstituted aromatic, a substituted or unsubstituted heteroaromatic, and any combination thereof, in any orientation; each Rₐ and R_{b} are independently selected from the group consisting of hydrogen, cyano, halogen, e.g., F, alkyl, halo-alkyl, —OH, —CO—, straight chain or branched alkoxy, straight chain or branched halo-alkyl, straight chain or branched halo-alkoxy, alkoxy-alkyl, hydroxyl-alkyl, -alkyl-hydroxyl, -alkyl-hydroxyl-alkyl, -halo-alkyl-hydroxyl-alkyl, -alkyl-hydroxyl-halo-alkyl, -halo-alkyl-hydroxyl-halo-alkyl, carboxy-alkyl, alkyl-SO₂, alkylcarbonyl, thioether, alkylsulfonyl, alkylcarbonylamino, alkylaminocarbonyl, alkyloxycarbonyl, alkylcarbonyloxy, substituted or unsubstituted aryl, and substituted or unsubstituted heteroaryl, all of which may be optionally substituted with OH, halogen, e.g., fluoro, straight chain or branched alkoxy, straight chain or branched halo-alkyl, straight chain and branched halo-alkoxy, alkoxy-alkyl, hydroxyl-alkyl, carboxy-alkyl, substituted or unsubstituted carbocyclic rings, and substituted or unsubstituted heterocyclic rings, which may contain one or more heteroatoms and may be saturated or unsaturated; or each Rₐ and R_{b} may form a 3-10-membered ring together with the carbon to which they are both attached;

each R₁ₐ and R₂ₐ are independently selected from the group consisting of hydrogen, cyano, halogen, alkyl, halo-alkyl, —OH, —CO—, straight chain or branched alkoxy, straight chain or branched halo-alkyl, straight chain or branched halo-alkoxy, alkoxy-alkyl, hydroxyl-alkyl, -alkyl-hydroxyl, -alkyl-hydroxyl-alkyl, -halo-alkyl-hydroxyl-alkyl, -alkyl-hydroxyl-halo-alkyl, -halo-alkyl-hydroxyl-halo-alkyl, carboxy-alkyl, alkyl-SO₂, alkylcarbonyl, thioether, alkylsulfonyl, alkylcarbonylamino, alkylaminocarbonyl, alkyloxycarbonyl, alkylcarbonyloxy, substituted or unsubstituted aryl, and substituted or unsubstituted heteroaryl, all of which may be optionally substituted with OH, halogen, e.g., fluoro, straight chain or branched alkoxy, straight chain or branched halo-alkyl, straight chain and branched halo-alkoxy, alkoxy-alkyl, hydroxyl-alkyl, carboxy-alkyl, substituted or unsubstituted carbocyclic rings, and substituted or unsubstituted heterocyclic rings, which may contain one or more heteroatoms and may be saturated or unsaturated; or each R₁ₐ and R₂ₐ may form a 3-10-membered ring together with the carbon to which they are both attached; and

each R¹⁴ and R¹⁵ is independently selected from the group consisting of hydrogen, halogen, cyano, straight chain or branched alkyl, straight chain or branched alkoxy, straight chain or branched halo-alkyl, straight chain or branched halo-alkoxy, alkoxy-alkyl, hydroxyl-alkyl, alkyl-SO₂, and carboxy-alkyl; or each R¹⁴ or R¹⁵ may form a 3-8-membered ring together with B₁, Rₐ, R_{b}, R₁ₐ, or R₂ₐ and the atoms to which they are attached. In certain embodiments, the total combination of each p and m is less than or equal to about 21, e.g., less than or equal to about 15, e.g., less than or equal to about 10, e.g., less than or equal to about 8, e.g., less than or equal to about 6.

An additional embodiment of the invention pertains to compounds of Formula XII having the following formula: 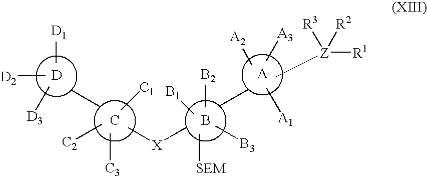
wherein:

SEM represents a selectivity enhancing moiety;

rings A, B, C, D are independently selected from the group consisting of any five- or six-membered aromatic or heteroaromatic, and isomers and tautomers thereof;

A₁, A₂, A₃, B₁, B₂, B₃, C₁, C₂, C₃, D₁, D₂, and D₃ are each independently selected from the group consisting of hydrogen, halogen, cyano, straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl, C₁-C₆-alkyl-SO₂ alkylcarbonyl, thioether, alkylsulfonyl, alkylcarbonylamino, alkylaminocarbonyl, alkyloxycarbonyl, alkylcarbonyloxy, substituted or unsubstituted aryl, substituted or unsubstituted heteroaryl, —OH, —CO₂-alkyl, —CO₂-halo-alkyl, —CONH₂, —CONH-alkyl, —CON-dialkyl, —CONH-halo-alkyl, —CON-halo-dialkyl, -alkyl-CONH-alkyl, -alkyl-CON-dialkyl, halo-alkyl-CONH-alkyl, halo-alkyl-CONH-halo-alkyl, alkyl-CONH-halo-dialkyl, —C₁-C₆-alkyl-hydroxyl, —C₁-C₆-alkyl-hydroxyl-alkyl (e.g., —CH₂OCH₃), —C₁-C₆-halo-alkyl-hydroxyl-alkyl (e.g., —CF₂OCH₃), —C₁-C₆-alkyl-hydroxyl-halo-alkyl(e.g., —CH₂OCF₃), —C₁-C₆-halo-alkyl-hydroxyl-halo-alkyl(e.g., —CF₂OCF₃), and N(R)R′; or taken together A₃ and B₃ may form a substituted or unsubstituted C₃-C₁₀ carbocyclic ring or substituted or unsubstituted C₃-C₁₀ heterocyclic ring, which may contain one or more heteroatoms and may be saturated or unsaturated; or taken together C₂ and D₂ may form a substituted or unsubstituted C₃-C₁₀ carbocyclic ring or substituted or unsubstituted C₃-C₁₀ heterocyclic ring, which may contain one or more heteroatoms and may be saturated or unsaturated;

R and R′ are each independently selected from the group consisting of hydrogen, halogen, cyano, straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, and carboxy-C₁-C₆-alkyl; or taken together R and R′ may form a substituted or unsubstituted C₃-C₁₀ carbocyclic ring or substituted or unsubstituted C₃-C₁₀ or heterocyclic ring, which may contain one or more heteroatoms and may be saturated or unsaturated; or taken together with the N to which they are attached R and R′ may form a moiety selected from the group consisting of substituted straight chain or cyclic guanyl, straight chain or cyclic guanidine, straight chain or cyclic urea, straight chain or cyclic thiourea, straight chain or cyclic carbamate, and straight chain or cyclic thiocarbamate;

Z is independently selected from the group consisting of C or N;

R¹ is a phospahate, a phosphate mimic or a phosphate precursor;

R² and R³ are each independently selected from the group consisting of hydrogen, halogen, cyano, straight chain or branched C¹-C₆-alkyl, alkyl-OR⁹, halo-alkyl-OR⁹, alkoxy-OR⁹, alkyl-OC(O)R⁹, halo-alkyl-OC(O)R⁹, alkoxy-OC(O)R⁹, alkyl-NR⁹R¹⁰, halo-alkyl-NR⁹R¹⁰, and alkoxy-NR⁹R¹⁰, all of which may be optionally substituted with OH, halogen, NHR⁹, NR⁹R¹⁰, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, or carboxy-C₁-C₆-alkyl; or taken together R² and R³ may form a substituted or unsubstituted C₃-C₁₀ carbocyclic ring or a substituted or unsubstituted C₃-C₁₀ heterocyclic ring, which may contain one or more heteroatoms and may be saturated or unsaturated; or taken together R² and A₁ may form a substituted or unsubstituted C₄-C₁₀ fused carbocyclic ring or substituted or unsubstituted C₄-C₁₀ fused heterocyclic rings, which may contain one or more heteroatoms and may be saturated or unsaturated;

R⁹ and R¹⁰ are independently selected from the group consisting of hydrogen, halogen, cyano, straight chain or branched C₁-C₆-alkyl, straight chain or branched C₁-C₆-alkoxy, straight chain or branched halo-C₁-C₆-alkyl, straight chain and branched halo-C₁-C₆-alkoxy, substituted or unsubstituted C₃-C₁₀ carbocyclic rings, and substituted or unsubstituted C₃-C₁₀ heterocyclic rings, which may contain one or more heteroatoms and may be saturated or unsaturated; or taken together R⁹ and R₁₀ may form a substituted or unsubstituted C₃-C₁₀ carbocyclic ring or a substituted or unsubstituted C₃-C₁₀ heterocyclic ring, which may contain one or more heteroatoms and may be saturated or unsaturated; or R⁹ or R¹⁰ together with A₁ may form a substituted or unsubstituted C₄-C₁₀ fused carbocyclic ring or substituted or unsubstituted C₄-C₁₀ fused heterocyclic rings, which may contain one or more heteroatoms and may be saturated or unsaturated;

X is selected from the group consisting of

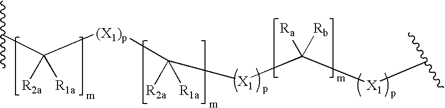

wherein each m is independently selected from an integer between 0 and 6; each p is independently selected from 0 or 1; each X₁ is independently selected from the group consisting of CR¹⁴R¹⁵, NR¹⁴, S, and O, —S(O), —S(O)₂, —OS(O)₂, —OS(O)₂O—, —C(O), C(OH), —C(O)O—, a substituted or unsubstituted aromatic, a substituted or unsubstituted heteroaromatic, and any combination thereof, in any orientation; each Rₐ and R_{b} are independently selected from the group consisting of hydrogen, cyano, and straight chain or branched C₁-C₆-alkyl, all of which may be optionally substituted with OH, halogen, e.g., fluoro, straight chain or branched C₁-C₆-alkoxy, straight chain or branched halo-C₁-C₆-alkyl, straight chain and branched halo-C₁-C₆-alkoxy, C₁-C₆-alkoxy-C₁-C₆-alkyl, hydroxyl-C₁-C₆-alkyl, carboxy-C₁-C₆-alkyl, substituted or unsubstituted C₃-C₁₀ carbocyclic rings, and substituted or unsubstituted C₃-C₁₀ heterocyclic rings, which may contain one or more heteroatoms and may be saturated or unsaturated; or each Rₐ and R_{b} may form a 3-8-membered ring together with the carbon to which they are both attached;

each R₁ₐ and R₂ₐ are independently selected from the group consisting of hydrogen, halogen, cyano, and straight chain or branched C₁-C₆-alkyl, all of which may be optionally substituted with OH, halogen, straight chain or branched C₁-C₆-alkoxy, straight chain or branched halo-C₁-C₆-alkyl, straight chain and branched halo-C₁-C₆-alkoxy, C₁-C₆-alkoxy-C₁-C₆-alkyl, hydroxyl-C₁-C₆-alkyl, carboxy-C₁-C₆-alkyl, substituted or unsubstituted C₃-C₁₀ carbocyclic rings, and substituted or unsubstituted C₃-C₁₀ heterocyclic rings, which may contain one or more heteroatoms and may be saturated or unsaturated; or each R₁ₐ and R₂ₐ may form a 3-8-membered ring together with the carbon to which they are both attached; and

each R¹⁴ and R¹⁵ is independently selected from the group consisting of hydrogen, halogen, cyano, straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, and carboxy-C₁-C₆-alkyl; or each R¹⁴ or R¹⁵ may form a 3-8-membered ring together with B₁, Rₐ, R_{b}, R₁ₐ, or R₂ₐ and the atoms to which they are attached.

In yet another embodiment, the invention relates to a compound of Formula XII having the following formula: 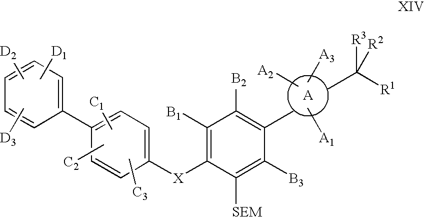
wherein:

SEM represents a selectivity enhancing moiety;

ring A is selected from the group consisting of any five- or six-membered aromatic or heteroaromatic, and isomers and tautomers thereof;

A₁, A₂, A₃, B₁, B₂, B₃, C₁, C₂, C₃, D₁, D₂, and D₃ are each independently selected from the group consisting of hydrogen, halogen, cyano, straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl, C₁-C₆-alkyl-SO₂ alkylcarbonyl, thioether, alkylsulfonyl, alkylcarbonylamino, alkylaminocarbonyl, alkyloxycarbonyl, alkylcarbonyloxy, substituted or unsubstituted aryl, substituted or unsubstituted heteroaryl, —OH, —CO₂-alkyl, —CO₂-halo-alkyl, —CONH₂, —CONH-alkyl, —CON-dialkyl, —CONH-halo-alkyl, —CON-halo-dialkyl, -alkyl-CONH-alkyl, -alkyl-CON-dialkyl, halo-alkyl-CONH-alkyl, halo-alkyl-CONH-halo-alkyl, alkyl-CONH-halo-dialkyl, —C₁-C₆-alkyl-hydroxyl, —C₁-C₆-alkyl-hydroxyl-alkyl, —C₁-C₆-halo-alkyl-hydroxyl-alkyl, —C₁-C₆-alkyl-hydroxyl-halo-alkyl, —C₁-C₆-halo-alkyl-hydroxyl-halo-alkyl, and N(R)R′; or taken together A₃ and B₃ may form a substituted or unsubstituted C₃-C₁₀ carbocyclic ring or substituted or unsubstituted C₃-C₁₀ heterocyclic ring, which may contain one or more heteroatoms and may be saturated or unsaturated; or taken together C₂ and D₂ may form a substituted or unsubstituted C₃-C₁₀ carbocyclic ring or substituted or unsubstituted C₃-C₁₀ heterocyclic ring, which may contain one or more heteroatoms and may be saturated or unsaturated;

R and R′ are each independently selected from the group consisting of hydrogen, halogen, cyano, straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, and carboxy-C₁-C₆-alkyl; or taken together R and R′ may form a substituted or unsubstituted C₃-C₁₀ carbocyclic ring or substituted or unsubstituted C₃-C₁₀ or heterocyclic ring, which may contain one or more heteroatoms and may be saturated or unsaturated; or taken together with the N to which they are attached R and R′ may form a moiety selected from the group consisting of substituted straight chain or cyclic guanyl, straight chain or cyclic guanidine, straight chain or cyclic urea, straight chain or cyclic thiourea, straight chain or cyclic carbamate, and straight chain or cyclic thiocarbamate;

R¹ is a phosphate, a phosphate mimic or a phosphate precursor;

R² and R³ are each independently selected from the group consisting of hydrogen, halogen, cyano, straight chain or branched C₁-C₆-alkyl, alkyl-OR⁹, halo-alkyl-OR⁹, alkoxy-OR⁹, alkyl-OC(O)R⁹, halo-alkyl-OC(O)R⁹, alkoxy-OC(O)R⁹, alkyl-NR⁹R¹⁰, halo-alkyl-NR⁹R¹⁰, and alkoxy-NR⁹R¹⁰, all of which may be optionally substituted with OH, halogen, NHR⁹, NR⁹R¹⁰, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, or carboxy-C₁-C₆-alkyl; or taken together R² and R³ may form a substituted or unsubstituted C₃-C₁₀ carbocyclic ring or a substituted or unsubstituted C₃-C₁₀ heterocyclic ring, which may contain one or more heteroatoms and may be saturated or unsaturated; or taken together R² and A₁ may form a substituted or unsubstituted C₄-C₁₀ fused carbocyclic ring or substituted or unsubstituted C₄-C₁₀ fused heterocyclic rings, which may contain one or more heteroatoms and may be saturated or unsaturated;

R⁹ and R¹⁰ are independently selected from the group consisting of hydrogen, halogen, cyano, straight chain or branched C₁-C₆-alkyl, straight chain or branched C₁-C₆-alkoxy, straight chain or branched halo-C₁-C₆-alkyl, straight chain and branched halo-C₁-C₆-alkoxy, substituted or unsubstituted C₃-C₁₀ carbocyclic rings, and substituted or unsubstituted C₃-C₁₀ heterocyclic rings, which may contain one or more heteroatoms and may be saturated or unsaturated; or taken together R⁹ and R¹⁰ may form a substituted or unsubstituted C₃-C₁₀ carbocyclic ring or a substituted or unsubstituted C₃-C₁₀ heterocyclic ring, which may contain one or more heteroatoms and may be saturated or unsaturated; or R⁹ or R¹⁰ together with A₁ may form a substituted or unsubstituted C₄-C₁₀ fused carbocyclic ring or substituted or unsubstituted C₄-C₁₀ fused heterocyclic rings, which may contain one or more heteroatoms and may be saturated or unsaturated;

X is selected from the group consisting of

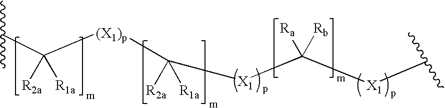

wherein each m is independently selected from an integer between 0 and 6; each p is independently selected from 0 or 1; each X₁ is independently selected from the group consisting of CR¹⁴R¹⁵, NR¹⁴, S, and O, —S(O), —S(O)₂, —OS(O)₂, —OS(O)₂O—, —C(O), C(OH), —C(O)O—, a substituted or unsubstituted aromatic, a substituted or unsubstituted heteroaromatic, and any combination thereof, in any orientation; each Rₐ and R_{b} are independently selected from the group consisting of hydrogen, cyano, and straight chain or branched C₁-C₆-alkyl, all of which may be optionally substituted with OH, halogen, e.g., fluoro, straight chain or branched C₁-C₆-alkoxy, straight chain or branched halo-C₁-C₆-alkyl, straight chain and branched halo-C₁-C₆-alkoxy, C₁-C₆-alkoxy-C₁-C₆-alkyl, hydroxyl-C₁-C₆-alkyl, carboxy-C₁-C₆-alkyl, substituted or unsubstituted C₃-C₁₀ carbocyclic rings, and substituted or unsubstituted C₃-C₁₀ heterocyclic rings, which may contain one or more heteroatoms and may be saturated or unsaturated; or each Rₐ and R_{b} may form a 3-8-membered ring together with the carbon to which they are both attached;

each R₁ₐ and R₂ₐ are independently selected from the group consisting of hydrogen, halogen, cyano, and straight chain or branched C₁-C₆-alkyl, all of which may be optionally substituted with OH, halogen, straight chain or branched C₁-C₆-alkoxy, straight chain or branched halo-C₁-C₆-alkyl, straight chain and branched halo-C₁-C₆-alkoxy, C₁-C₆-alkoxy-C₁-C₆-alkyl, hydroxyl-C₁-C₆-alkyl, carboxy-C₁-C₆-alkyl, substituted or unsubstituted C₃-C₁₀ carbocyclic rings, and substituted or unsubstituted C₃-C₁₀ heterocyclic rings, which may contain one or more heteroatoms and may be saturated or unsaturated; or each R₁ₐ and R₂ₐ may form a 3-8-membered ring together with the carbon to which they are both attached; and

each R¹⁴ and R¹⁵ is independently selected from the group consisting of hydrogen, halogen, cyano, straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C-C₆-alkyl, and carboxy-C₁-C₆-alkyl; or each R¹⁴ or R¹⁵ may form a 3-8-membered ring together with B₁, SEM, Rₐ, R_{b}, R₁ₐ, or R₂ₐ and the atoms to which they are attached. In a particular embodiment, A is selected from the group consisting of an aromatic ring and a heteroaromatic ring.

An additional embodiment of the invention is directed to a compound of Formula XII having the following formula: 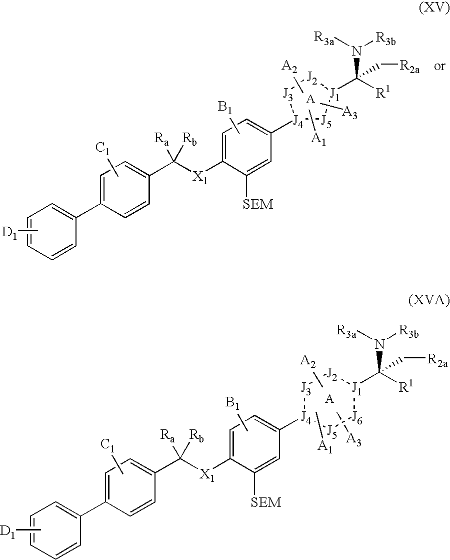
wherein:

the dashed lines represent a single or double bond;

SEM represents a selectivity enhancing moiety;

A₁, A₂, A₃, B₁, C₁, and D₁, are each independently selected from the group consisting of hydrogen, halogen, cyano, straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl, C₁-C₆-alkyl-SO₂ alkylcarbonyl, thioether, alkylsulfonyl, alkylcarbonylamino, alkylaminocarbonyl, alkyloxycarbonyl, alkylcarbonyloxy, substituted or unsubstituted aryl, substituted or unsubstituted heteroaryl, —OH, —CO₂-alkyl, —CO₂-halo-alkyl, —CONH₂, —CONH-alkyl, —CON-dialkyl, —CONH-halo-alkyl, —CON-halo-dialkyl, -alkyl-CONH-alkyl, -alkyl-CON-dialkyl, halo-alkyl-CONH-alkyl, halo-alkyl-CONH-halo-alkyl, alkyl-CONH-halo-dialkyl, —C₁-C₆-alkyl-hydroxyl, —C₁-C₆-alkyl-hydroxyl-alkyl, —C₁-C₆-halo-alkyl-hydroxyl-alkyl, —C₁-C₆-alkyl-hydroxyl-halo-alkyl, —C₁-C₆-halo-alkyl-hydroxyl-halo-alkyl, and N(R)R′; or taken together A₃ and B₁ may form a substituted or unsubstituted C₃-C₁₀ carbocyclic ring or substituted or unsubstituted C₃-C₁₀ heterocyclic ring, which may contain one or more heteroatoms and may be saturated or unsaturated; or taken together C₁ and D₁ may form a substituted or unsubstituted C₃-C₁₀ carbocyclic ring or substituted or unsubstituted C₃-C₁₀ heterocyclic ring, which may contain one or more heteroatoms and may be saturated or unsaturated;

R and R′ are each independently selected from the group consisting of hydrogen, halogen, cyano, straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, and carboxy-C₁-C₆-alkyl; or taken together R and R′ may form a substituted or unsubstituted C₃-C₁₀ carbocyclic ring or substituted or unsubstituted C₃-C₁₀ or heterocyclic ring, which may contain one or more heteroatoms and may be saturated or unsaturated; or taken together with the N to which they are attached R and R′ may form a moiety selected from the group consisting of substituted straight chain or cyclic guanyl, straight chain or cyclic guanidine, straight chain or cyclic urea, straight chain or cyclic thiourea, straight chain or cyclic carbamate, and straight chain or cyclic thiocarbamate;

J₁, J₂, J₃, J₄, J₅, and J₆ are independently selected from the group consisting of C, CH, N, NH, O, and S;

R¹ is a phosphate, a phosphate mimic or a phosphate precursor;

R^{2a} is selected from the group consisting of hydrogen, halogen, cyano, straight chain or branched C₁-C₆-alkyl, and straight chain or branched halo-C₁-C₆-alkyl, all of which may be optionally substituted with OH, halogen, —OR⁹, or —OC(O)R⁹;

R₃ₐ and R_{3b} are each independently selected from the group consisting of hydrogen, halogen, cyano, straight chain or branched C₁-C₆-alkyl, straight chain or branched halo-C₁-C₆-alkyl; or taken together R₃ₐ and R_{3b} may form a group selected from the group consisting of C₃-C₆-carbocycle and C₃-C₆-halo-carbocycle;

R⁹ is selected from the group consisting of hydrogen, halogen, cyano, straight chain or branched C₁-C₆-alkyl, straight chain or branched C₁-C₆-alkoxy, straight chain or branched halo-C₁-C₆-alkyl, straight chain and branched halo-C₁-C₆-alkoxy, substituted or unsubstituted C₃-C₁₀ carbocyclic rings, and substituted or unsubstituted C₃-C₁₀ heterocyclic rings, which may contain one or more heteroatoms and may be saturated or unsaturated;

X₁ is selected from the group consisting of CR¹⁴R¹⁵, NR¹⁴, S, and O, —S(O), —S(O)₂, —OS(O)₂, —OS(O)₂O—, —C(O), C(OH), —C(O)O—, any five- or six-membered aromatic or heteroaromatic, isomers and tautomers thereof, and any combination thereof, in any orientation;

Rₐ and R_{b} are each independently selected from the group consisting of hydrogen, halogen, alkyl, halo-alkyl, —OH, —CO—, straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, —C₁-C₆-alkyl-hydroxyl, —C₁-C₆-alkyl-hydroxyl-alkyl, —C₁-C₆-halo-alkyl-hydroxyl-alkyl, —C₁-C₆-alkyl-hydroxyl-halo-alkyl, —C₁-C₆-halo-alkyl-hydroxyl-halo-alkyl, carboxy-C₁-C₆-alkyl, C₁-C₆-alkyl-SO₂, alkylcarbonyl, thioether, alkylsulfonyl, alkylcarbonylamino, alkylaminocarbonyl, alkyloxycarbonyl, alkylcarbonyloxy, substituted or unsubstituted aryl, and substituted or unsubstituted heteroaryl; or taken together Rₐ and R_{b} may form a C₃-C₆-carbocycle, C₁-C₆-halo-carbocycle, substituted or unsubstituted C₃-C₁₀ carbocyclic rings and substituted or unsubstituted C₃-C₁₀ heterocyclic rings, which may contain one or more heteroatoms and may be saturated or unsaturated;

each R¹⁴ and R¹⁵ is independently selected from the group consisting of hydrogen, halogen, cyano, straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, and carboxy-C₁-C₆-alkyl; or each R¹⁴ or R¹⁵ may form a 3-8-membered ring together with B₁, Rₐ, or R_{b}, and the atoms to which they are attached. In a particular embodiment of Formula XV, A is selected from the group consisting of a 5-membered aromatic ring and a 5-membered heteroaromatic ring.

In another embodiment, the invention pertains to a compound of Formula XII having the following formula: 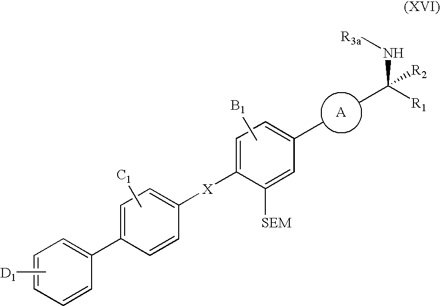
wherein:

SEM represents a selectivity enhancing moiety;

ring A is selected from the group consisting of any five- or six-membered aromatic or heteroaromatic, and isomers and tautomers thereof;

R¹ is a phosphate, a phosphate mimic or a phosphate precursor;

R₂ is selected from the group consisting of —H, —F, —CN, —OH, —CH₂OH, —CHFOH, CF₂OH, CH(CH₃)OH, CF(CH₃)OH, CH(CF₃)OH, —CH₃, —CH₂CH₃, —CF₃, —CF₂CF₃, cyclopropyl, fluorinated cyclopropyl, —CH₂OR⁹, —CH₂OC(O)R⁹,

R⁹ is selected from a group consisting of straight chain or branched C₁-C₆-alkyl, straight chain or branched C₁-C₆-alkoxy, straight chain or branched halo-C₁-C₆-alkyl, straight chain and branched halo-C₁-C₆-alkoxy, substituted or unsubstituted C₃-C₁₀ carbocyclic rings, and substituted or unsubstituted C₃-C₁₀ heterocyclic rings, which may contain one or more heteroatoms and may be saturated or unsaturated;

X is selected from the group consisting of

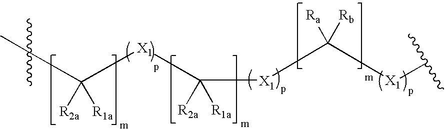

wherein each m is independently selected from an integer between 0 and 6; each p is independently selected from 0 or 1; each X₁ is independently selected from the group consisting of CR¹⁴R¹⁵, NR¹⁴, S, and O, —S(O), —S(O)₂, —OS(O)₂, —OS(O)₂O—, —C(O), C(OH), —C(O)O—, a substituted or unsubstituted aromatic, a substituted or unsubstituted heteroaromatic, and any combination thereof, in any orientation; each Rₐ and R_{b} are independently selected from the group consisting of hydrogen, cyano, halogen, alkyl, halo-alkyl, —OH—, —CO—, straight chain or branched alkoxy, straight chain or branched halo-alkyl, straight chain or branched halo-alkoxy, alkoxy-alkyl, hydroxyl-alkyl, -alkyl-hydroxyl, -alkyl-hydroxyl-alkyl, -halo-alkyl-hydroxyl-alkyl, -alkyl-hydroxyl-halo-alkyl, -halo-alkyl-hydroxyl-halo-alkyl, carboxy-alkyl, alkyl-SO₂, alkylcarbonyl, thioether, alkylsulfonyl, alkylcarbonylamino, alkylaminocarbonyl, alkyloxycarbonyl, alkylcarbonyloxy, substituted or unsubstituted aryl, and substituted or unsubstituted heteroaryl, all of which may be optionally substituted with OH, halogen, e.g., fluoro, straight chain or branched alkoxy, straight chain or branched halo-alkyl, straight chain and branched halo-alkoxy, alkoxy-alkyl, hydroxyl-alkyl, carboxy-alkyl, substituted or unsubstituted carbocyclic rings, and substituted or unsubstituted heterocyclic rings, which may contain one or more heteroatoms and may be saturated or unsaturated; or each Rₐ and R_{b} may form a 3-10-membered ring together with the carbon to which they are both attached;

each R₁ₐ and R₂ₐ are independently selected from the group consisting of hydrogen, cyano, halogen, alkyl, halo-alkyl, —OH, —CO—, straight chain or branched alkoxy, straight chain or branched halo-alkyl, straight chain or branched halo-alkoxy, alkoxy-alkyl, hydroxyl-alkyl, -alkyl-hydroxyl, -alkyl-hydroxyl-alkyl, -halo-alkyl-hydroxyl-alkyl, -alkyl-hydroxyl-halo-alkyl, -halo-alkyl-hydroxyl-halo-alkyl, carboxy-alkyl, alkyl-SO₂, alkylcarbonyl, thioether, alkylsulfonyl, alkylcarbonylamino, alkylaminocarbonyl, alkyloxycarbonyl, alkylcarbonyloxy, substituted or unsubstituted aryl, and substituted or unsubstituted heteroaryl, all of which may be optionally substituted with OH, halogen, e.g., fluoro, straight chain or branched alkoxy, straight chain or branched halo-alkyl, straight chain and branched halo-alkoxy, alkoxy-alkyl, hydroxyl-alkyl, carboxy-alkyl, substituted or unsubstituted carbocyclic rings, and substituted or unsubstituted heterocyclic rings, which may contain one or more heteroatoms and may be saturated or unsaturated; or each R₁ₐ and R₂ₐ may form a 3-10-membered ring together with the carbon to which they are both attached; and

each R¹⁴ and R¹⁵ is independently selected from the group consisting of hydrogen, halogen, cyano, straight chain or branched alkyl, straight chain or branched alkoxy, straight chain or branched halo-alkyl, straight chain or branched halo-alkoxy, alkoxy-alkyl, hydroxyl-alkyl, alkyl-SO₂, and carboxy-alkyl; or each R¹⁴ or R¹⁵ may form a 3-8-membered ring together with B₁, SEM, Rₐ, R_{b}, R₁ₐ, or R₂ₐ and the atoms to which they are attached.

R₃ₐ is selected from the group consisting of consisting of —H, straight chain or branched C₁-C₆-alkyl, straight chain or branched halo-C₁-C₆-alkyl, substituted or unsubstituted C₃-C₁₀ carbocyclic rings and substituted or unsubstituted C₃-C₁₀ heterocyclic rings, —C(O)alkyl, —C(O)NH-alkyl, —C(O)N-dialkyl, —C(O)aryl, —C(O)NH-aryl, —C(O)N-alkyl-aryl, —C(O)N-diaryl, —C(O)heteroaryl, —C(O)NH-heteroaryl, —C(O)N-carbocycle;

D₁, C₁, and B₁ are each independently selected from the group consisting of —H, —F, —Cl, —Br, —I, -alkyl, -halo-alkyl, —CN, —COR¹⁶, —CH₂OR⁶, —CHFOR¹⁶, CF₂OR¹⁶, —OR¹⁶, alkylcarbonyl, thioether, alkylsulfonyl, alkylcarbonylamino, alkylaminocarbonyl, alkyloxycarbonyl, alkylcarbonyloxy, substituted or unsubstituted aromatic , substituted or unsubstituted heteroaromatic, straight chain or branched alkylene, straight chain or branched alkenyl, straight chain or branched alkynyl, straight chain or branched alkenylene, arylalkyl, alkylaryl, alkylene-aryl, alkenyl-aryl, alkynyl-aryl, and alkenylene-aryl groups, and —N(R¹⁶)R¹⁷, aryl; and

R¹⁶ and R¹⁷ are each independently selected from the group consisting of hydrogen, straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl, and C₁-C₆-alkyl-SO₂. In particular embodiments, the SEM is selected from the group consisting of —F, —Cl, —Br, —I, -halo-alkyl, —CN, —COR¹⁸, —CH₂OR¹⁸, —CHFOR¹⁸, CF₂OR¹⁸, —OR, —N(R¹⁸)R¹⁹, aryl, alkylcarbonyl, thioether, alkylsulfonyl, alkylcarbonylamino, alkylaminocarbonyl, alkyloxycarbonyl, alkylcarbonyloxy, substituted or unsubstituted aromatic, substituted or unsubstituted heteroaromatic, straight chain or branched alkyl, straight chain or branched alkenyl, straight chain or branched alkynyl, straight chain or branched alkenyl, arylalkyl, alkylaryl, alkenyl-aryl, and alkynyl-aryl, groups; wherein R⁸ and R¹⁹ are each independently selected from hydrogen, straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl or C₁-C₆-alkyl-SO₂. In other particular embodiments, A is selected from the group consisting of an aromatic ring and a heteroaromatic ring. For example, ring A may be selected from the group consisting of

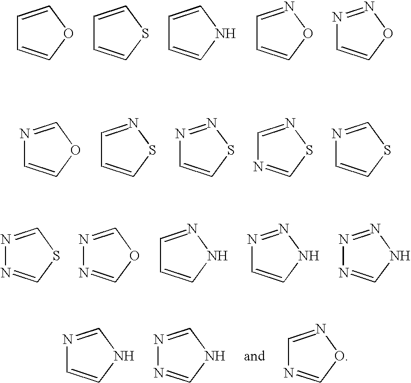

In another embodiment, the invention is directed to a compound of Formula XVI having the following formula: 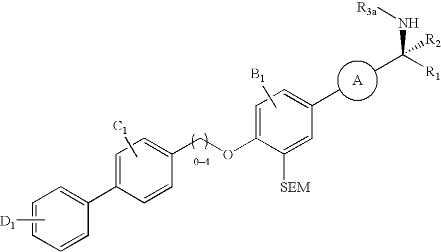
wherein SEM, B₁, C₁, D₁, R₁, R₂, and R₃ₐ are as defined for Formula XVI.

Another embodiment of the invention relates to a compound of Formula XVII: 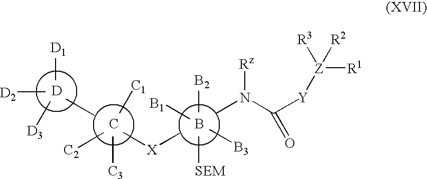
wherein:

SEM represents a selectivity enhancing moiety;

rings B, C, D are independently selected from the group consisting of substituted or unsubstituted carbocyclic rings and substituted or unsubstituted heterocyclic rings, which may contain one or more heteroatoms and may be saturated or unsaturated;

B₁, B₂, B₃, C₁, C₂, C₃, D₁, D₂, and D₃ are each independently selected from the group consisting of hydrogen, halogen, cyano, straight chain or branched aliphatic, straight chain or branched alkoxy, straight chain or branched halo-alkyl, straight chain or branched halo-alkoxy, alkoxy-alkyl, hydroxyl-alkyl, carboxy-alkyl, alkyl-SO₂ alkylcarbonyl, thioether, alkylsulfonyl, alkylcarbonylamino, alkylaminocarbonyl, alkyloxycarbonyl, alkylcarbonyloxy, substituted or unsubstituted aryl, substituted or unsubstituted heteroaryl, —OH, —C(O)-alkyl, —C(O)-halo-alkyl, —C(O)O-alkyl, —C(O)O-halo-alkyl, —CONH₂, —CONH-alkyl, —CON-dialkyl, —CONH-halo-alkyl, —CON-halo-dialkyl, -alkyl-CONH-alkyl, -alkyl-CON-dialkyl, halo-alkyl-CONH-alkyl, halo-alkyl-CONH-halo-alkyl, alkyl-CONH-halo-dialkyl, -alkyl-hydroxyl, -alkyl-hydroxyl-alkyl, -halo-alkyl-hydroxyl-alkyl, -alkyl-hydroxyl-halo-alkyl, -halo-alkyl-hydroxyl-halo-alkyl, substituted or unsubstituted alkyl-OR¹⁴, substituted or unsubstituted haloalkyl-OR⁴, —OR¹⁴, and N(R)R′; or taken together R^{Z} and B₃ may form a substituted or unsubstituted carbocyclic ring or substituted or unsubstituted heterocyclic ring, which may contain one or more heteroatoms and may be saturated or unsaturated; or taken together C₂ and D₂ may form a substituted or unsubstituted carbocyclic ring or substituted or unsubstituted heterocyclic ring, which may contain one or more heteroatoms and may be saturated or unsaturated;

R and R′ are each independently selected from the group consisting of hydrogen, cyano, straight chain or branched alkyl, straight chain or branched alkoxy, straight chain or branched halo-alkyl, straight chain or branched halo-alkoxy, alkoxy-alkyl, hydroxyl-alkyl, and carboxy-alkyl; or taken together R and R′ may form a substituted or unsubstituted carbocyclic ring or substituted or unsubstituted or heterocyclic ring, which may contain one or more heteroatoms and may be saturated or unsaturated; or taken together with the N to which they are attached R and R′ may form a moiety selected from the group consisting of substituted straight chain or cyclic guanyl, straight chain or cyclic guanidine, straight chain or cyclic urea, straight chain or cyclic thiourea, straight chain or cyclic carbamate, and straight chain or cyclic thiocarbamate;

Z is independently selected from the group consisting of C or N;

R¹ is a phosphate derivative, a phosphate mimic or a phosphate precursor;

R² and R³ are each independently selected from the group consisting of hydrogen, hydroxyl, halogen, cyano, straight chain or branched alkyl, alkyl-OR⁹, halo-alkyl-OR⁹, alkoxy-OR⁹, alkyl-OC(O)R⁹, halo-alkyl-OC(O)R⁹, alkoxy-OC(O)R⁹, carbocyclic rings, heterocyclic rings which may contain one or more heteroatoms, alkyl-NR⁹R¹⁰, halo-alkyl-NR⁹R¹⁰, and alkoxy-NR⁹R¹⁰, all of which may be optionally substituted with OH, halogen, NHR⁹, NR⁹R¹⁰, straight chain or branched alkoxy, straight chain or branched halo-alkyl, straight chain or branched halo-alkoxy, alkoxy-alkyl, hydroxyl-alkyl, or carboxy-alkyl; or taken together R² and R³ may form a substituted or unsubstituted carbocyclic ring or a substituted or unsubstituted heterocyclic ring, which may contain one or more heteroatoms and may be saturated or unsaturated; or taken together R² and R^{Z} may form a substituted or unsubstituted C₄-C₁₀ fused carbocyclic ring or substituted or unsubstituted C₄-C₁₀ fused heterocyclic rings, which may contain one or more heteroatoms and may be saturated or unsaturated;

R⁹ and R¹⁰ are independently selected from the group consisting of hydrogen, halogen, cyano, straight chain or branched alkyl, straight chain or branched alkoxy, straight chain or branched halo-alkyl, straight chain and branched halo-alkoxy, —C(O)alkyl, —C(O)NH-alkyl, —C(O)N-dialkyl, —C(O)aryl, —C(O)NH-aryl, —C(O)N-alkyl-aryl, —C(O)N-diaryl, —C(O)heteroaryl, —C(O)NH-heteroaryl, —C(O)N-carbocycle, substituted or unsubstituted carbocyclic rings, and substituted or unsubstituted heterocyclic rings, which may contain one or more heteroatoms and may be saturated or unsaturated; or taken together R⁹ and R¹⁰ may form a substituted or unsubstituted carbocyclic ring or a substituted or unsubstituted heterocyclic ring, which may contain one or more heteroatoms and may be saturated or unsaturated;

Y is independently selected from the group consisting of (CR¹¹R¹²), and (CR¹¹R¹²)ₙNR¹³;

R¹¹, R¹², and R¹³ are independently selected from the group consisting of hydrogen, halogen, cyano, and straight chain or branched alkyl, all of which may be optionally substituted with OH, halogen, straight chain or branched alkoxy, straight chain or branched halo-alkyl, and straight chain and branched halo-alkoxy; or R¹³ may form a 3-8-membered ring together with either R¹¹ or R¹² and the atom to which they are attached;

n is an integer from 0 to 3;

X is selected from the group consisting of

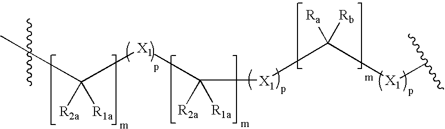

wherein each m is independently selected from an integer between 0 and 6; each p is independently selected from 0 or 1; each X₁ is independently selected from the group consisting of CR¹⁴R¹⁵, NR¹⁴, S, and O, —S(O), —S(O)₂, —OS(O)₂, —OS(O)₂O—, —C(O), C(OH), —C(O)O—, a substituted or unsubstituted aromatic, a substituted or unsubstituted heteroaromatic, and any combination thereof, in any orientation; each Rₐ and R_{b} are independently selected from the group consisting of hydrogen, cyano, halogen, alkyl, halo-alkyl, —OH, —CO—, straight chain or branched alkoxy, straight chain or branched halo-alkyl, straight chain or branched halo-alkoxy, alkoxy-alkyl, hydroxyl-alkyl, -alkyl-hydroxyl, -alkyl-hydroxyl-alkyl, -halo-alkyl-hydroxyl-alkyl, -alkyl-hydroxyl-halo-alkyl, -halo-alkyl-hydroxyl-halo-alkyl, carboxy-alkyl, alkyl-SO₂, alkylcarbonyl, thioether, alkylsulfonyl, alkylcarbonylamino, alkylaminocarbonyl, alkyloxycarbonyl, alkylcarbonyloxy, substituted or unsubstituted aryl, and substituted or unsubstituted heteroaryl, all of which may be optionally substituted with OH, halogen, e.g., fluoro, straight chain or branched alkoxy, straight chain or branched halo-alkyl, straight chain and branched halo-alkoxy, alkoxy-alkyl, hydroxyl-alkyl, carboxy-alkyl, substituted or unsubstituted carbocyclic rings, and substituted or unsubstituted heterocyclic rings, which may contain one or more heteroatoms and may be saturated or unsaturated; or each Rₐ and R_{b} may form a 3-10-membered ring together with the carbon to which they are both attached;

each R₁ₐ and R₂ₐ are independently selected from the group consisting of hydrogen, cyano, halogen, alkyl, halo-alkyl, —OH, —CO—, straight chain or branched alkoxy, straight chain or branched halo-alkyl, straight chain or branched halo-alkoxy, alkoxy-alkyl, hydroxyl-alkyl, -alkyl-hydroxyl, -alkyl-hydroxyl-alkyl, -halo-alkyl-hydroxyl-alkyl, -alkyl-hydroxyl-halo-alkyl, -halo-alkyl-hydroxyl-halo-alkyl, carboxy-alkyl, alkyl-SO₂, alkylcarbonyl, thioether, alkylsulfonyl, alkylcarbonylamino, alkylaminocarbonyl, alkyloxycarbonyl, alkylcarbonyloxy, substituted or unsubstituted aryl, and substituted or unsubstituted heteroaryl, all of which may be optionally substituted with OH, halogen, e.g., fluoro, straight chain or branched alkoxy, straight chain or branched halo-alkyl, straight chain and branched halo-alkoxy, alkoxy-alkyl, hydroxyl-alkyl, carboxy-alkyl, substituted or unsubstituted carbocyclic rings, and substituted or unsubstituted heterocyclic rings, which may contain one or more heteroatoms and may be saturated or unsaturated; or each R₁ₐ and R₂ₐ may form a 3-10-membered ring together with the carbon to which they are both attached;

each R¹⁴ and R¹⁵ is independently selected from the group consisting of hydrogen, halogen, cyano, straight chain or branched alkyl, straight chain or branched alkoxy, straight chain or branched halo-alkyl, straight chain or branched halo-alkoxy, alkoxy-alkyl, hydroxyl-alkyl, alkyl-SO₂, and carboxy-alkyl; or each R¹⁴ or R¹⁵ may form a 3-8-membered ring together with B₁, Rₐ, R_{b}, R₁ₐ, or R₂ₐ and the atoms to which they are attached; and

R^{Z} selected from the group consisting of hydrogen, cyano, straight chain or branched alkyl, cycloalkyl, straight chain or branched alkoxy, straight chain or branched halo-alkyl, halo-cycloalkyl, straight chain or branched halo-alkoxy, alkoxy-alkyl, hydroxyl-alkyl, alkyl-SO₂, and carboxy-alkyl; or R^{Z} may form a 3-8-membered ring together with B₁, R² or R³ and the atoms to which they are attached. In certain embodiments, the total combination of each p and m is less than or equal to about 21, e.g., less than or equal to about 15, e.g., less than or equal to about 10, e.g., less than or equal to about 8, e.g., less than or equal to about 6.

In one embodiment, the invention pertains in part to a compound of Formulae XVIII-XX: 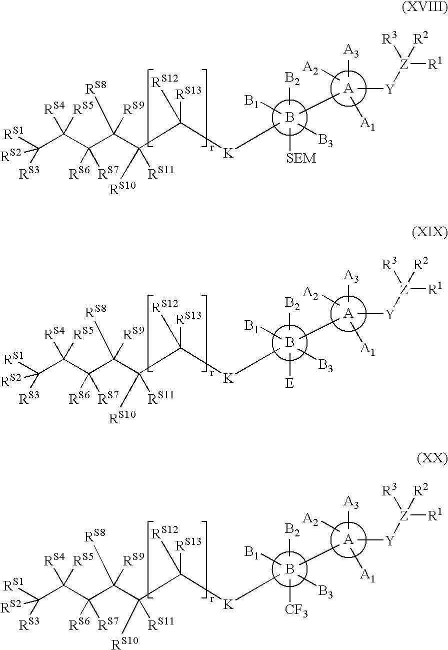
wherein:

SEM is selected from a group consisting of cyano, straight chain or branched C₁-C₆-alkyl, straight chain or-branched C₁-C₆-alkoxy, straight chain or branched halo-C₁-C₆-alkyl (e.g. CF₃), straight chain or branched halo-C₁-C₆-alkoxy, C₁-C₆-alkoxy-C₁-C₆-alkyl, hydroxyl-C₁-C₆-alkyl, carboxy-C₁-C₆-alkyl, C₁-C₆-alkyl-SO₂ or N(R)R′, wherein R and R′ are each independently hydrogen, straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl or C₁-C₆-alkyl-SO₂; alkylcarbonyl, thioether, alkylsulfonyl, alkylcarbonylamino, alkylaminocarbonyl, alkyloxycarbonyl, alkylcarbonyloxy, substituted or unsubstituted aromatic, substituted or unsubstituted heteroaromatic, any straight chain or branched alkylene, straight chain or branched alkenyl, straight chain or branched alkynyl, straight chain or branched alkenylene, arylalkyl, alkylaryl, alkylene-aryl, alkenyl-aryl, alkynyl-aryl or alkenylene-aryl group.;

rings A and B are independently selected from the group consisting of substituted or unsubstituted carbocyclic rings and substituted or unsubstituted heterocyclic rings, which may contain one or more heteroatoms and may be saturated or unsaturated;

A₁, A₂, A₃, B₁, B₂, and B₃ are each independently selected from the group consisting of hydrogen, halogen, cyano, straight chain or branched aliphatic, straight chain or branched alkoxy, straight chain or branched halo-alkyl, straight chain or branched halo-alkoxy, alkoxy-alkyl, hydroxyl-alkyl, carboxy-alkyl, alkyl-SO₂ alkylcarbonyl, thioether, alkylsulfonyl, alkylcarbonylamino, alkylaminocarbonyl, alkyloxycarbonyl, alkylcarbonyloxy, substituted or unsubstituted aryl, substituted or unsubstituted heteroaryl, —OH, —C(O)-alkyl, —C(O)-halo-alkyl, —C(O)O-alkyl, —C(O)O-halo-alkyl, —CONH₂, —CONH-alkyl, —CON-dialkyl, —CONH-halo-alkyl, —CON-halo-dialkyl, -alkyl-CONH-alkyl, -alkyl-CON-dialkyl, halo-alkyl-CONH-alkyl, halo-alkyl-CONH-halo-alkyl, alkyl-CONH-halo-dialkyl, -alkyl-hydroxyl, -alkyl-hydroxyl-alkyl, -halo-alkyl-hydroxyl-alkyl, -alkyl-hydroxyl-halo-alkyl, -halo-alkyl-hydroxyl-halo-alkyl, substituted or unsubstituted alkyl-OR¹⁴, substituted or unsubstituted haloalkyl-OR¹⁴, —OR¹⁴, and N(R)R′; or taken together A₃ and B₃ may form a substituted or unsubstituted carbocyclic ring or substituted or unsubstituted heterocyclic ring, which may contain one or more heteroatoms and may be saturated or unsaturated;

R is independently selected from the group consisting of hydrogen, cyano, straight chain or branched alkyl, straight chain or branched alkoxy, straight chain or branched halo-alkyl, straight chain or branched halo-alkoxy, alkoxy-alkyl, hydroxyl-alkyl, and carboxy-alkyl; or taken together R and R′ may form a substituted or unsubstituted carbocyclic ring or substituted or unsubstituted or heterocyclic ring, which may contain one or more heteroatoms and may be saturated or unsaturated; or taken together with the N to which they are attached R and R′ may form a moiety selected from the group consisting of substituted straight chain or cyclic guanyl, straight chain or cyclic guanidine, straight chain or cyclic urea, straight chain or cyclic thiourea, straight chain or cyclic carbamate, and straight chain or cyclic thiocarbamate;

R′ is independently selected from the group consisting of cyano, straight chain or branched alkyl, straight chain or branched alkoxy, straight chain or branched halo-alkyl, straight chain or branched halo-alkoxy, alkoxy-alkyl, hydroxyl-alkyl, and carboxy-alkyl; or taken together R and R′ may form a substituted or unsubstituted carbocyclic ring or substituted or unsubstituted or heterocyclic ring, which may contain one or more heteroatoms and may be saturated or unsaturated; or taken together with the N to which they are attached R and R′ may form a moiety selected from the group consisting of substituted straight chain or cyclic guanyl, straight chain or cyclic guanidine, straight chain or cyclic urea, straight chain or cyclic thiourea, straight chain or cyclic carbamate, and straight chain or cyclic thiocarbamate;

Z is independently selected from the group consisting of C or N;

R¹ is a phosphate derivative, a phosphate mimic or a phosphate precursor;

R² and R³ are each independently selected from the group consisting of hydrogen, hydroxyl, halogen, cyano, straight chain or branched alkyl, alkyl-OR⁹, halo-alkyl-OR⁹, alkoxy-OR⁹, alkyl-OC(O)R⁹, halo-alkyl-OC(O)R⁹, alkoxy-OC(O)R⁹, carbocyclic rings, heterocyclic rings which may contain one or more heteroatoms, alkyl-NR⁹R¹⁰, halo-alkyl-NR⁹R¹⁰, and alkoxy-NR⁹R¹⁰, all of which may be optionally substituted with OH, halogen, NHR⁹, NR⁹R¹⁰, straight chain or branched alkoxy, straight chain or branched halo-alkyl, straight chain or branched halo-alkoxy, alkoxy-alkyl, hydroxyl-alkyl, or carboxy-alkyl; or taken together R² and R³ may form a substituted or unsubstituted carbocyclic ring or a substituted or unsubstituted heterocyclic ring, which may contain one or more heteroatoms and may be saturated or unsaturated; or taken together R² and A₁ may form a substituted or unsubstituted C₄-C₁₀ fused carbocyclic ring or substituted or unsubstituted C₄-C₁₀ fused heterocyclic rings, which may contain one or more heteroatoms and may be saturated or unsaturated;

R⁹ and R¹⁰ are independently selected from the group consisting of hydrogen, halogen, cyano, straight chain or branched alkyl, straight chain or branched alkoxy, straight chain or branched halo-alkyl, straight chain and branched halo-alkoxy, —C(O)alkyl, —C(O)NH-alkyl, —C(O)N-dialkyl, —C(O)aryl, —C(O)NH-aryl, —C(O)N-alkyl-aryl, —C(O)N-diaryl, —C(O)heteroaryl, —C(O)NH-heteroaryl, —C(O)N-carbocycle, substituted or unsubstituted carbocyclic rings, and substituted or unsubstituted heterocyclic rings, which may contain one or more heteroatoms and may be saturated or unsaturated; or taken together R⁹ and R¹⁰ may form a substituted or unsubstituted carbocyclic ring or a substituted or unsubstituted heterocyclic ring, which may contain one or more heteroatoms and may be saturated or unsaturated; or R⁹ or R¹⁰ together with A₁ may form a substituted or unsubstituted C₄-C₁₀ fused carbocyclic ring or substituted or unsubstituted C₄-C₁₀ fused heterocyclic rings, which may contain one or more heteroatoms and may be saturated or unsaturated;

Y is independently selected from the group consisting of (CR¹¹R¹²)ₙ and (CR¹¹R¹²)ₙNR¹³;

R¹¹, R¹², and R¹³ are independently selected from the group consisting of hydrogen, halogen, cyano, and straight chain or branched alkyl, all of which may be optionally substituted with OH, halogen, straight chain or branched alkoxy, straight chain or branched halo-alkyl, and straight chain and branched halo-alkoxy; or R¹³ may form a 3-8-membered ring together with either R¹¹ or R¹² and the atom to which they are attached;

n is an integer from 0 to 3;

r is an integer from 0 to 7;

E is haloalkyl;

K is selected from the group consisting of

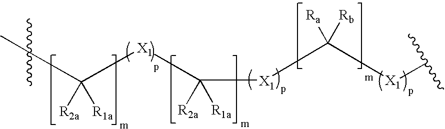

wherein each m is independently selected from an integer between 0 and 6; each p is independently selected from 0 or 1; each X₁ is independently selected from the group consisting of CR¹⁴R¹⁵, NR¹⁴, S, and O, —S(O), —S(O)₂, —OS(O)₂, —OS(O)₂O—, —C(O), C(OH), —C(O)O—, a substituted or unsubstituted aromatic, a substituted or unsubstituted heteroaromatic, and any combination thereof, in any orientation; each Rₐ and R_{b} are independently selected from the group consisting of hydrogen, cyano, and straight chain or branched C₁-C₆-alkyl, all of which may be optionally substituted with OH, halogen, e.g., fluoro, straight chain or branched C₁-C₆-alkoxy, straight chain or branched halo-C₁-C₆-alkyl, straight chain and branched halo-C₁-C₆-alkoxy, C₁-C₆-alkoxy-C₁-C₆-alkyl, hydroxyl-C₁-C₆-alkyl, carboxy-C₁-C₆-alkyl, substituted or unsubstituted C₃-C₁₀ carbocyclic rings, and substituted or unsubstituted C₃-C₁₀ heterocyclic rings, which may contain one or more heteroatoms and may be saturated or unsaturated; or each Rₐ and R_{b} may form a 3-8-membered ring together with the carbon to which they are both attached;

R₁ₐ and R₂ₐ are independently selected from the group consisting of hydrogen, halogen, cyano, and straight chain or branched C₁-C₆-alkyl, all of which may be optionally substituted with OH, halogen, straight chain or branched C₁-C₆-alkoxy, straight chain or branched halo-C₁-C₆-alkyl, straight chain and branched halo-C₁-C₆-alkoxy, C₁-C₆-alkoxy-C₁-C₆-alkyl, hydroxyl-C₁-C₆-alkyl, carboxy-C₁-C₆-alkyl, substituted or unsubstituted C₃-C₁₀ carbocyclic rings, and substituted or unsubstituted C₃-C₁₀ heterocyclic rings, which may contain one or more heteroatoms and may be saturated or unsaturated; or each R₁ₐ and R₂ₐ may form a 3-8-membered ring together with the carbon to which they are both attached;

R¹⁴ and R¹⁵ are independently selected from the group consisting of hydrogen, halogen, cyano, straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, and carboxy-C₁-C₆-alkyl; or each R¹⁴ or R¹⁵ may form a 3-8-membered ring together with B₁, SEM, Rₐ, R_{b}, R₁ₐ, or R₂ₐ and the atoms to which they are attached; and

R^{S1} through R^{S11}, each R^{S12}, and each R^{S13} are each independently selected from the group consisting of hydrogen, cyano, halogen, alkyl, halo-alkyl, —OH, —CO—, straight chain or branched alkoxy, straight chain or branched halo-alkyl, straight chain or branched halo-alkoxy, alkoxy-alkyl, hydroxyl-alkyl, -alkyl-hydroxyl, -alkyl-hydroxyl-alkyl, -halo-alkyl-hydroxyl-alkyl, -alkyl-hydroxyl-halo-alkyl, -halo-alkyl-hydroxyl-halo-alkyl, carboxy-alkyl, alkyl-SO₂, alkylcarbonyl, thioether, alkylsulfonyl, alkylcarbonylamino, alkylaminocarbonyl, alkyloxycarbonyl, alkylcarbonyloxy, substituted or unsubstituted aryl, and substituted or unsubstituted heteroaryl, all of which may be optionally substituted with OH, halogen, e.g., fluoro, straight chain or branched alkoxy, straight chain or branched halo-alkyl, straight chain and branched halo-alkoxy, alkoxy-alkyl, hydroxyl-alkyl, carboxy-alkyl, substituted or unsubstituted carbocyclic rings, and substituted or unsubstituted heterocyclic rings, which may contain one or more heteroatoms and may be saturated or unsaturated; or taken together any two of R^{S1} through R^{S13} may form a 3-8-membered ring together with the carbon to which they are both attached and any intervening carbons on the associated chain. In certain embodiments, the SEM is trifluoromethyl and R^{S2} is C₁-C₆ alkyl. In certain embodiments, ring B is a phenyl moiety. In certain embodiments, the total combination of each p and m is less than or equal to about 21, e.g., less than or equal to about 15, e.g., less than or equal to about 10, e.g., less than or equal to about 8, e.g., less than or equal to about 6. In certain embodiments, one of R^{S1}, R^{S2}, or R^{S3} is selected from the group consisting of substituted or unsubstituted carbocyclic rings, e.g., cyclohexyl, and substituted or unsubstituted heterocyclic rings, which may contain one or more heteroatoms and may be saturated or unsaturated. In certain embodiments, r is 3.

Another embodiment of the invention relates to a compound of Formulae XXI-XXIII: 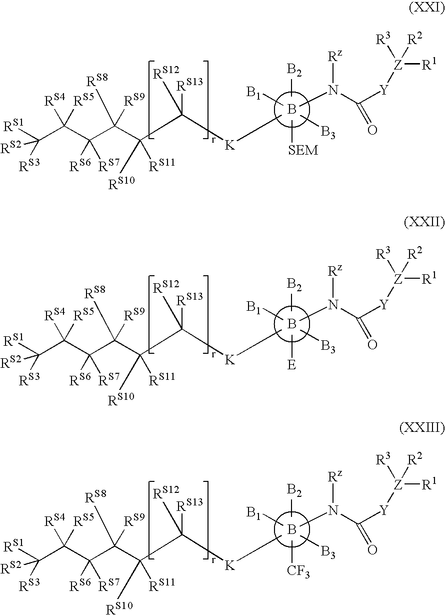
wherein:

SEM is selected from a group consisting of cyano, straight chain or branched halo-C₁-C₆-alkyl (e.g. CF₃), straight chain or branched halo-C₁-C₆-alkoxy, C₁-C₆-alkoxy-C₁-C₆-alkyl, hydroxyl-C₁-C₆-alkyl, carboxy-C₁-C₆-alkyl, C₁-C₆-alkyl-SO₂ or N(R)R′, wherein R and R′ are each independently hydrogen, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl or C₁-C₆-alkyl-SO₂; alkylcarbonyl, thioether, alkylsulfonyl, alkylcarbonylamino, alkylaminocarbonyl, alkyloxycarbonyl, alkylcarbonyloxy, substituted or unsubstituted aromatic, substituted or unsubstituted heteroaromatic, any straight chain or branched alkylene, straight chain or branched alkenyl, straight chain or branched alkynyl, straight chain or branched alkenylene, arylalkyl, alkylaryl, alkylene-aryl, alkenyl-aryl, alkynyl-aryl or alkenylene-aryl group;

ring B is selected from the group consisting of substituted or unsubstituted carbocyclic rings and substituted or unsubstituted heterocyclic rings, which may contain one or more heteroatoms and may be saturated or unsaturated;

B₁, B₂, and B₃ are each independently selected from the group consisting of hydrogen, halogen, cyano, straight chain or branched aliphatic, straight chain or branched alkoxy, straight chain or branched halo-alkyl, straight chain or branched halo-alkoxy, alkoxy-alkyl, hydroxyl-alkyl, carboxy-alkyl, alkyl-SO₂ alkylcarbonyl, thioether, alkylsulfonyl, alkylcarbonylamino, alkylaminocarbonyl, alkyloxycarbonyl, alkylcarbonyloxy, substituted or unsubstituted aryl, substituted or unsubstituted heteroaryl, —OH, —C(O)-alkyl, —C(O)-halo-alkyl, —C(O)O-alkyl, —C(O)O-halo-alkyl, —CONH₂, —CONH-alkyl, —CON-dialkyl, —CONH-halo-alkyl, —CON-halo-dialkyl, -alkyl-CONH-alkyl, -alkyl-CON-dialkyl, halo-alkyl-CONH-alkyl, halo-alkyl-CONH-halo-alkyl, alkyl-CONH-halo-dialkyl, -alkyl-hydroxyl, -alkyl-hydroxyl-alkyl, -halo-alkyl-hydroxyl-alkyl, -alkyl-hydroxyl-halo-alkyl, -halo-alkyl-hydroxyl-halo-alkyl, substituted or unsubstituted alkyl-OR¹⁴, substituted or unsubstituted haloalkyl-OR¹⁴, —OR⁴, and N(R)R′; or taken together R^{Z} and B₃ may form a substituted or unsubstituted carbocyclic ring or substituted or unsubstituted heterocyclic ring, which may contain one or more heteroatoms and may be saturated or unsaturated;

R is independently selected from the group consisting of hydrogen, cyano, straight chain or branched alkyl, straight chain or branched alkoxy, straight chain or branched halo-alkyl, straight chain or branched halo-alkoxy, alkoxy-alkyl, hydroxyl-alkyl, and carboxy-alkyl; or taken together R and R′ may form a substituted or unsubstituted carbocyclic ring or substituted or unsubstituted or heterocyclic ring, which may contain one or more heteroatoms and may be saturated or unsaturated; or taken together with the N to which they are attached R and R′ may form a moiety selected from the group consisting of substituted straight chain or cyclic guanyl, straight chain or cyclic guanidine, straight chain or cyclic urea, straight chain or cyclic thiourea, straight chain or cyclic carbamate, and straight chain or cyclic thiocarbamate;

R′ is independently selected from the group consisting of cyano, straight chain or branched alkyl, straight chain or branched alkoxy, straight chain or branched halo-alkyl, straight chain or branched halo-alkoxy, alkoxy-alkyl, hydroxyl-alkyl, and carboxy-alkyl; or taken together R and R′ may form a substituted or unsubstituted carbocyclic ring or substituted or unsubstituted or heterocyclic ring, which may contain one or more heteroatoms and may be saturated or unsaturated; or taken together with the N to which they are attached R and R′ may form a moiety selected from the group consisting of substituted straight chain or cyclic guanyl, straight chain or cyclic guanidine, straight chain or cyclic urea, straight chain or cyclic thiourea, straight chain or cyclic carbamate, and straight chain or cyclic thiocarbamate;

Z is independently selected from the group consisting of C or N;

R¹ is a phosphate derivative, a phosphate mimic or a phosphate precursor;

R² and R³ are each independently selected from the group consisting of hydrogen, hydroxyl, halogen, cyano, straight chain or branched alkyl, alkyl-OR⁹, halo-alkyl-OR⁹, alkoxy-OR⁹, alkyl-OC(O)R⁹, halo-alkyl-OC(O)R⁹, alkoxy-OC(O)R⁹, carbocyclic rings, heterocyclic rings which may contain one or more heteroatoms, alkyl-NR⁹R¹⁰, halo-alkyl-NR⁹R¹⁰, and alkoxy-NR⁹R¹⁰, all of which may be optionally substituted with OH, halogen, NHR⁹, NR⁹R¹⁰, straight chain or branched alkoxy, straight chain or branched halo-alkyl, straight chain or branched halo-alkoxy, alkoxy-alkyl, hydroxyl-alkyl, or carboxy-alkyl; or taken together R² and R³ may form a substituted or unsubstituted carbocyclic ring or a substituted or unsubstituted heterocyclic ring, which may contain one or more heteroatoms and may be saturated or unsaturated; or taken together R² and R^{Z} may form a substituted or unsubstituted C₄-C₁₀ fused carbocyclic ring or substituted or unsubstituted C₄-C₁₀ fused heterocyclic rings, which may contain one or more heteroatoms and may be saturated or unsaturated;

R⁹ and R¹⁰ are independently selected from the group consisting of hydrogen, halogen, cyano, straight chain or branched alkyl, straight chain or branched alkoxy, straight chain or branched halo-alkyl, straight chain and branched halo-alkoxy, —C(O)alkyl, —C(O)NH-alkyl, —C(O)N-dialkyl, —C(O)aryl, —C(O)NH-aryl, —C(O)N-alkyl-aryl, —C(O)N-diaryl, —C(O)heteroaryl, —C(O)NH-heteroaryl, —13 C(O)N-carbocycle, substituted or unsubstituted carbocyclic rings, and substituted or unsubstituted heterocyclic rings, which may contain one or more heteroatoms and may be saturated or unsaturated; or taken together R⁹ and R¹⁰ may form a substituted or unsubstituted carbocyclic ring or a substituted or unsubstituted heterocyclic ring, which may contain one or more heteroatoms and may be saturated or unsaturated;

Y is independently selected from the group consisting of (CR¹¹R¹²)ₙ and (CR¹¹R¹²)ₙNR¹³;

R¹¹, R¹², and R¹³ are independently selected from the group consisting of hydrogen, halogen, cyano, and straight chain or branched alkyl, all of which may be optionally substituted with OH, halogen, straight chain or branched alkoxy, straight chain or branched halo-alkyl, and straight chain and branched halo-alkoxy; or R¹³ may form a 3-8-membered ring together with either R¹¹ or R¹² and the atom to which they are attached;

n is an integer from 0 to 3;

r is an integer from 0 to 7;

E is haloalkyl;

K is selected from the group consisting of

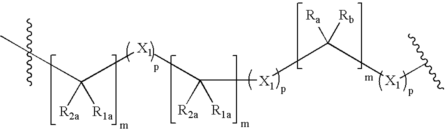

wherein each m is independently selected from an integer between 0 and 6; each p is independently selected from 0 or 1; each X₁ is independently selected from the group consisting of CR¹⁴R¹⁵, NR¹⁴, S, and O, —S(O), —S(O)₂, —OS(O)₂, —OS(O)₂O—, —C(O), C(OH), —C(O)O—, a substituted or unsubstituted aromatic, a substituted or unsubstituted heteroaromatic, and any combination thereof, in any orientation; each Rₐ and R_{b} are independently selected from the group consisting of hydrogen, cyano, and straight chain or branched C₁-C₆-alkyl, all of which may be optionally substituted with OH, halogen, e.g., fluoro, straight chain or branched C₁-C₆-alkoxy, straight chain or branched halo-C₁-C₆-alkyl, straight chain and branched halo-C₁-C₆-alkoxy, C₁-C₆-alkoxy-C₁-C₆-alkyl, hydroxyl-C₁-C₆-alkyl, carboxy-C₁-C₆-alkyl, substituted or unsubstituted C₃-C₁₀ carbocyclic rings, and substituted or unsubstituted C₃-C₁₀ heterocyclic rings, which may contain one or more heteroatoms and may be saturated or unsaturated; or each Rₐ and R_{b} may form a 3-8-membered ring together with the carbon to which they are both attached;

R₁ₐ and R₂ₐ are independently selected from the group consisting of hydrogen, halogen, cyano, and straight chain or branched C₁-C₆-alkyl, all of which may be optionally substituted with OH, halogen, straight chain or branched C₁-C₆-alkoxy, straight chain or branched halo-C₁-C₆-alkyl, straight chain and branched halo-C₁-C₆-alkoxy, C₁-C₆-alkoxy-C₁-C₆-alkyl, hydroxyl-C₁-C₆-alkyl, carboxy-C₁-C₆-alkyl, substituted or unsubstituted C₃-C₁₀ carbocyclic rings, and substituted or unsubstituted C₃-C₁₀ heterocyclic rings, which may contain one or more heteroatoms and may be saturated or unsaturated; or each R₁ₐ and R₂ₐ may form a 3-8-membered ring together with the carbon to which they are both attached;

R¹⁴ and R¹⁵ are independently selected from the group consisting of hydrogen, halogen, cyano, straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, and carboxy-C₁-C₆-alkyl; or each R¹⁴ or R¹⁵ may form a 3-8-membered ring together with B₁, SEM, Rₐ, R_{b}, R₁ₐ, or R₂ₐ and the atoms to which they are attached;

R^{S1} through R^{S11}, each R^{S12}, and each R^{S13} are each independently selected from the group consisting of hydrogen, cyano, halogen, alkyl, halo-alkyl, —OH, —CO—, straight chain or branched alkoxy, straight chain or branched halo-alkyl, straight chain or branched halo-alkoxy, alkoxy-alkyl, hydroxyl-alkyl, -alkyl-hydroxyl, -alkyl-hydroxyl-alkyl, -halo-alkyl-hydroxyl-alkyl, -alkyl-hydroxyl-halo-alkyl, -halo-alkyl-hydroxyl-halo-alkyl, carboxy-alkyl, alkyl-SO₂, alkylcarbonyl, thioether, alkylsulfonyl, alkylcarbonylamino, alkylaminocarbonyl, alkyloxycarbonyl, alkylcarbonyloxy, substituted or unsubstituted aryl, and substituted or unsubstituted heteroaryl, all of which may be optionally substituted with OH, halogen, e.g., fluoro, straight chain or branched alkoxy, straight chain or branched halo-alkyl, straight chain and branched halo-alkoxy, alkoxy-alkyl, hydroxyl-alkyl, carboxy-alkyl, substituted or unsubstituted carbocyclic rings, and substituted or unsubstituted heterocyclic rings, which may contain one or more heteroatoms and may be saturated or unsaturated; or taken together any two of R^{S1} through R^{S13} may form a 3-8-membered ring together with the carbon to which they are both attached and any intervening carbons on the associated chain; and

R^{Z} selected from the group consisting of hydrogen, halogen, cyano, straight chain or branched alkyl, cycloalkyl, straight chain or branched alkoxy, straight chain or branched halo-alkyl, halo-cycloalkyl, straight chain or branched halo-alkoxy, alkoxy-alkyl, hydroxyl-alkyl, alkyl-SO₂, and carboxy-alkyl; or R^{Z} may form a 3-8-membered ring together with B₁, R² or R³ and the atoms to which they are attached. In certain embodiments, the SEM is trifluoromethyl and R^{S2} is C₁-C₆ alkyl. In certain embodiments, ring B is a phenyl moiety. In certain embodiments, the total combination of each p and m is less than or equal to about 21, e.g., less than or equal to about 15, e.g., less than or equal to about 10, e.g., less than or equal to about 8, e.g., less than or equal to about 6. In certain embodiments, r is 3.

In another embodiment, the invention relates to compounds of Formulae XXIV-XXXV: 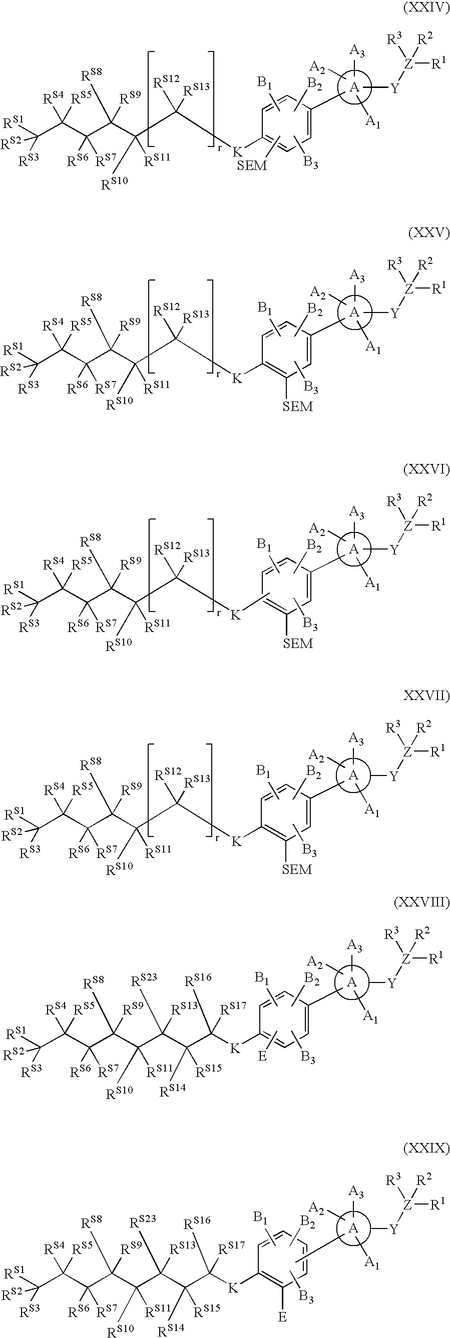 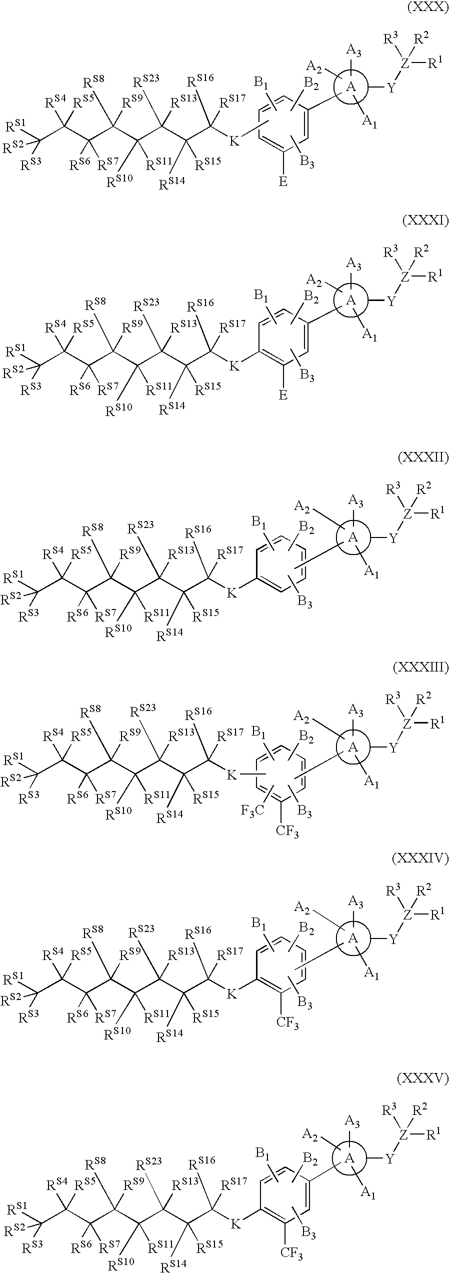
wherein:

SEM is selected from a group consisting of halogen (e.g., Br), cyano, straight chain or branched C₁-C₆-alkyl, straight chain or branched C₁-C₆-alkoxy, straight chain or branched halo-C₁-C₆-alkyl (e.g., CF₃), straight chain or branched halo-C₁-C₆-alkoxy, C₁-C₆-alkoxy-C₁-C₆-alkyl, hydroxyl-C₁-C₆-alkyl, carboxy-C₁-C₆-alkyl, C₁-C₆-alkyl-SO₂ or N(R)R′, wherein R and R′ are each independently hydrogen, straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl or C₁-C₆-alkyl-SO₂; alkylcarbonyl, thioether, alkylsulfonyl, alkylcarbonylamino, alkylaminocarbonyl, alkyloxycarbonyl, alkylcarbonyloxy, substituted or unsubstituted aromatic, substituted or unsubstituted heteroaromatic, any straight chain or branched alkylene, straight chain or branched alkenyl, straight chain or branched alkynyl, straight chain or branched alkenylene, arylalkyl, alkylaryl, alkylene-aryl, alkenyl-aryl, alkynyl-aryl or alkenylene-aryl group;

ring A is selected from the group consisting of substituted or unsubstituted carbocyclic rings and substituted or unsubstituted heterocyclic rings, which may contain one or more heteroatoms and may be saturated or unsaturated;

A₁, A₂, A₃, B₁, B₂, and B₃ are each independently selected from the group consisting of hydrogen, halogen, cyano, straight chain or branched aliphatic, straight chain or branched alkoxy, straight chain or branched halo-alkyl, straight chain or branched halo-alkoxy, alkoxy-alkyl, hydroxyl-alkyl, carboxy-alkyl, alkyl-SO₂ alkylcarbonyl, thioether, alkylsulfonyl, alkylcarbonylamino, alkylaminocarbonyl, alkyloxycarbonyl, alkylcarbonyloxy, substituted or unsubstituted aryl, substituted or unsubstituted heteroaryl, —OH, —C(O)-alkyl, —C(O)-halo-alkyl, —C(O)O-alkyl, —C(O)O-halo-alkyl, —CONH₂, —CONH-alkyl, —CON-dialkyl, —CONH-halo-alkyl, —CON-halo-dialkyl, -alkyl-CONH-alkyl, -alkyl-CON-dialkyl, halo-alkyl-CONH-alkyl, halo-alkyl-CONH-halo-alkyl, alkyl-CONH-halo-dialkyl, -alkyl-hydroxyl, -alkyl-hydroxyl-alkyl, -halo-alkyl-hydroxyl-alkyl, -alkyl-hydroxyl-halo-alkyl, -halo-alkyl-hydroxyl-halo-alkyl, substituted or unsubstituted alkyl-OR¹⁴, substituted or unsubstituted haloalkyl-OR¹⁴, —OR¹⁴, and N(R)R′; or taken together A₃ and B₃ may form a substituted or unsubstituted carbocyclic ring or substituted or unsubstituted heterocyclic ring, which may contain one or more heteroatoms and may be saturated or unsaturated;

R is independently selected from the group consisting of hydrogen, cyano, straight chain or branched alkyl, straight chain or branched alkoxy, straight chain or branched halo-alkyl, straight chain or branched halo-alkoxy, alkoxy-alkyl, hydroxyl-alkyl, and carboxy-alkyl; or taken together R and R′ may form a substituted or unsubstituted carbocyclic ring or substituted or unsubstituted or heterocyclic ring, which may contain one or more heteroatoms and may be saturated or unsaturated; or taken together with the N to which they are attached R and R′ may form a moiety selected from the group consisting of substituted straight chain or cyclic guanyl, straight chain or cyclic guanidine, straight chain or cyclic urea, straight chain or cyclic thiourea, straight chain or cyclic carbamate, and straight chain or cyclic thiocarbamate;

R′ is independently selected from the group consisting of cyano, straight chain or branched alkyl, straight chain or branched alkoxy, straight chain or branched halo-alkyl, straight chain or branched halo-alkoxy, alkoxy-alkyl, hydroxyl-alkyl, and carboxy-alkyl; or taken together R and R′ may form a substituted or unsubstituted carbocyclic ring or substituted or unsubstituted or heterocyclic ring, which may contain one or more heteroatoms and may be saturated or unsaturated; or taken together with the N to which they are attached R and R′ may form a moiety selected from the group consisting of substituted straight chain or cyclic guanyl, straight chain or cyclic guanidine, straight chain or cyclic urea, straight chain or cyclic thiourea, straight chain or cyclic carbamate, and straight chain or cyclic thiocarbamate;

Z is independently selected from the group consisting of C or N;

R¹ is a phosphate derivative, a phosphate mimic or a phosphate precursor;

R² and R³ are each independently selected from the group consisting of hydrogen, hydroxyl, halogen, cyano, straight chain or branched alkyl, alkyl-OR⁹, halo-alkyl-OR⁹, alkoxy-OR⁹, alkyl-OC(O)R⁹, halo-alkyl-OC(O)R⁹, alkoxy-OC(O)R⁹, carbocyclic rings, heterocyclic rings which may contain one or more heteroatoms, alkyl-NR⁹R¹⁰, halo-alkyl-NR⁹R¹⁰, and alkoxy-NR⁹R¹⁰, all of which may be optionally substituted with OH, halogen, NHR⁹, NR⁹R¹⁰, straight chain or branched alkoxy, straight chain or branched halo-alkyl, straight chain or branched halo-alkoxy, alkoxy-alkyl, hydroxyl-alkyl, or carboxy-alkyl; or taken together R² and R³ may form a substituted or unsubstituted carbocyclic ring or a substituted or unsubstituted heterocyclic ring, which may contain one or more heteroatoms and may be saturated or unsaturated; or taken together R² and A₁ may form a substituted or unsubstituted C₄-C₁₀ fused carbocyclic ring or substituted or unsubstituted C₄-C₁₀ fused heterocyclic rings, which may contain one or more heteroatoms and may be saturated or unsaturated;

R⁹ and R¹⁰ are independently selected from the group consisting of hydrogen, halogen, cyano, straight chain or branched alkyl, straight chain or branched alkoxy, straight chain or branched halo-alkyl, straight chain and branched halo-alkoxy, —C(O)alkyl, —C(O)NH-alkyl, —C(O)N-dialkyl, —C(O)aryl, —C(O)NH-aryl, —C(O)N-alkyl-aryl, —C(O)N-diaryl, —C(O)heteroaryl, —C(O)NH-heteroaryl, —C(O)N-carbocycle, substituted or unsubstituted carbocyclic rings, and substituted or unsubstituted heterocyclic rings, which may contain one or more heteroatoms and may be saturated or unsaturated; or taken together R⁹ and R¹⁰ may form a substituted or unsubstituted carbocyclic ring or a substituted or unsubstituted heterocyclic ring, which may contain one or more heteroatoms and may be saturated or unsaturated; or R⁹ or R¹⁰ together with A₁ may form a substituted or unsubstituted C₄-C₁₀ fused carbocyclic ring or substituted or unsubstituted C₄-C₁₀ fused heterocyclic rings, which may contain one or more heteroatoms and may be saturated or unsaturated;

Y is independently selected from the group consisting of (CR¹¹R¹²)ₙ and (CR¹¹R¹²)ₙNR¹³;

R¹¹, R¹², and R¹³ are independently selected from the group consisting of hydrogen, halogen, cyano, and straight chain or branched alkyl, all of which may be optionally substituted with OH, halogen, straight chain or branched alkoxy, straight chain or branched halo-alkyl, and straight chain and branched halo-alkoxy; or R¹³ may form a 3-8-membered ring together with either R¹¹ or R¹² and the atom to which they are attached;

n is an integer from 0 to 3;

r is an integer from 0 to 7;

E is haloalkyl;

K is selected from the group consisting of

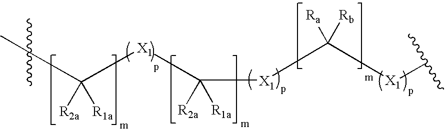

wherein each m is independently selected from an integer between 0 and 6; each p is independently selected from 0 or 1; each X₁ is independently selected from the group consisting of CR¹⁴R¹⁵, NR¹⁴, S, and O, —S(O), —S(O)₂, —OS(O)₂, —OS(O)₂O—, —C(O), C(OH), —C(O)O—, a substituted or unsubstituted aromatic, a substituted or unsubstituted heteroaromatic, and any combination thereof, in any orientation; each Rₐ and R_{b} are independently selected from the group consisting of hydrogen, cyano, and straight chain or branched C₁-C₆-alkyl, all of which may be optionally substituted with OH, halogen, e.g., fluoro, straight chain or branched C₁-C₆-alkoxy, straight chain or branched halo-C₁-C₆-alkyl, straight chain and branched halo-C₁-C₆-alkoxy, C₁-C₆-alkoxy-C₁-C₆-alkyl, hydroxyl-C₁-C₆-alkyl, carboxy-C₁-C₆-alkyl, substituted or unsubstituted C₃-C₁₀ carbocyclic rings, and substituted or unsubstituted C₃-C₁₀ heterocyclic rings, which may contain one or more heteroatoms and may be saturated or unsaturated; or each Rₐ and R_{b} may form a 3-8-membered ring together with the carbon to which they are both attached;

R₁ₐ and R₂ₐ are independently selected from the group consisting of hydrogen, halogen, cyano, and straight chain or branched C₁-C₆-alkyl, all of which may be optionally substituted with OH, halogen, straight chain or branched C₁-C₆-alkoxy, straight chain or branched halo-C₁-C₆-alkyl, straight chain and branched halo-C₁-C₆-alkoxy, C₁-C₆-alkoxy-C₁-C₆-alkyl, hydroxyl-C₁-C₆-alkyl, carboxy-C₁-C₆-alkyl, substituted or unsubstituted C₃-C₁₀ carbocyclic rings, and substituted or unsubstituted C₃-C₁₀ heterocyclic rings, which may contain one or more heteroatoms and may be saturated or unsaturated; or each R₁ₐ and R₂ₐ may form a 3-8-membered ring together with the carbon to which they are both attached;

R¹⁴ and R¹⁵ are independently selected from the group consisting of hydrogen, halogen, cyano, straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, and carboxy-C₁-C₆-alkyl; or each R¹⁴ or R¹⁵ may form a 3-8-membered ring together with B₁, SEM, Rₐ, R_{b}, R₁ₐ, or R₂ₐ and the atoms to which they are attached; and

R^{S1} through R^{S17} are each independently selected from the group consisting of hydrogen, cyano, halogen, alkyl, halo-alkyl, —OH, —CO—, straight chain or branched alkoxy, straight chain or branched halo-alkyl, straight chain or branched halo-alkoxy, alkoxy-alkyl, hydroxyl-alkyl, -alkyl-hydroxyl, -alkyl-hydroxyl-alkyl, -halo-alkyl-hydroxyl-alkyl, -alkyl-hydroxyl-halo-alkyl, -halo-alkyl-hydroxyl-halo-alkyl, carboxy-alkyl, alkyl-SO₂, alkylcarbonyl, thioether, alkylsulfonyl, alkylcarbonylamino, alkylaminocarbonyl, alkyloxycarbonyl, alkylcarbonyloxy, substituted or unsubstituted aryl, and substituted or unsubstituted heteroaryl, all of which may be optionally substituted with OH, halogen, e.g., fluoro, straight chain or branched alkoxy, straight chain or branched halo-alkyl, straight chain and branched halo-alkoxy, alkoxy-alkyl, hydroxyl-alkyl, carboxy-alkyl, substituted or unsubstituted carbocyclic rings, and substituted or unsubstituted heterocyclic rings, which may contain one or more heteroatoms and may be saturated or unsaturated. In certain embodiments of Formulae XXIVY-XXVII, the SEM is selected from a group consisting of cyano, straight chain or branched C₁-C₆-alkyl, straight chain or-branched C₁-C₆-alkoxy, straight chain or branched halo-C₁-C₆-alkyl (e.g., CF₃), straight chain or branched halo-C₁-C₆-alkoxy, C₁-C₆-alkoxy-C₁-C₆-alkyl, hydroxyl-C₁-C₆-alkyl, carboxy-C₁-C₆-alkyl, C₁-C₆-alkyl-SO₂ or N(R)R′, wherein R and R′ are each independently hydrogen, straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl or C₁-C₆-alkyl-SO₂; alkylcarbonyl, thioether, alkylsulfonyl, alkylcarbonylamino, alkylaminocarbonyl, alkyloxycarbonyl, alkylcarbonyloxy, substituted or unsubstituted aromatic, substituted or unsubstituted heteroaromatic, any straight chain or branched alkylene, straight chain or branched alkenyl, straight chain or branched alkynyl, straight chain or branched alkenylene, arylalkyl, alkylaryl, alkylene-aryl, alkenyl-aryl, alkynyl-aryl or alkenylene-aryl group. In particular embodiments, the SEM is trifluoromethyl and R^{S2} is C₁-C₆ alkyl. In certain embodiments, the total combination of each p and m is less than or equal to about 21, e.g., less than or equal to about 15, e.g., less than or equal to about 10, e.g., less than or equal to about 8, e.g., less than or equal to about 6. In certain embodiments, r is 3.

The invention also relates to compounds of Formulae XXXVI-XLVII: 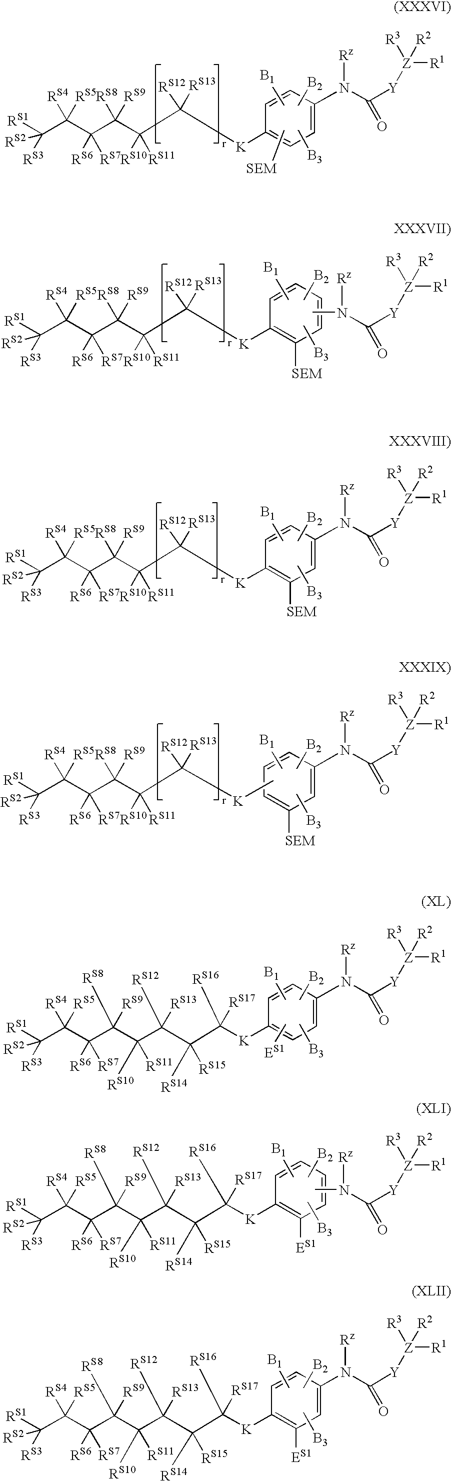 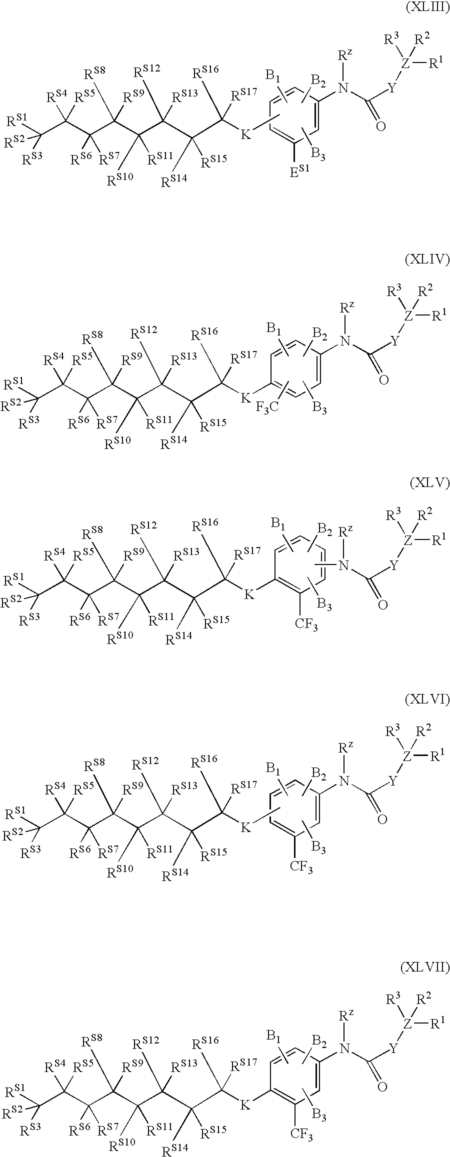
wherein:

SEM is selected from a group consisting of halogen (e.g., Br), cyano, straight chain or branched C₁-C₆-alkyl, straight chain or branched C₁-C₆-alkoxy, straight chain or branched halo-C₁-C₆-alkyl (e.g., CF₃), straight chain or branched halo-C₁-C₆-alkoxy, C₁-C₆-alkoxy-C₁-C₆-alkyl, hydroxyl-C₁-C₆-alkyl, carboxy-C₁-C₆-alkyl, C₁-C₆-alkyl-SO₂ or N(R)R′, wherein R and R′ are each independently hydrogen, straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl or C₁-C₆-alkyl-SO₂; alkylcarbonyl, thioether, alkylsulfonyl, alkylcarbonylamino, alkylaminocarbonyl, alkyloxycarbonyl, alkylcarbonyloxy, substituted or unsubstituted aromatic, substituted or unsubstituted heteroaromatic, any straight chain or branched alkylene, straight chain or branched alkenyl, straight chain or branched alkynyl, straight chain or branched alkenylene, arylalkyl, alkylaryl, alkylene-aryl, alkenyl-aryl, alkynyl-aryl or alkenylene-aryl group;

ring B is selected from the group consisting of substituted or unsubstituted carbocyclic rings and substituted or unsubstituted heterocyclic rings, which may contain one or more heteroatoms and may be saturated or unsaturated;

B₁, B₂, and B₃ are each independently selected from the group consisting of hydrogen, halogen, cyano, straight chain or branched aliphatic, straight chain or branched alkoxy, straight chain or branched halo-alkyl, straight chain or branched halo-alkoxy, alkoxy-alkyl, hydroxyl-alkyl, carboxy-alkyl, alkyl-SO₂ alkylcarbonyl, thioether, alkylsulfonyl, alkylcarbonylamino, alkylaminocarbonyl, alkyloxycarbonyl, alkylcarbonyloxy, substituted or unsubstituted aryl, substituted or unsubstituted heteroaryl, —OH, —C(O)-alkyl, —C(O)-halo-alkyl, —C(O)O-alkyl, —C(O)O-halo-alkyl, —CONH₂, —CONH-alkyl, —CON-dialkyl, —CONH-halo-alkyl, —CON-halo-dialkyl, —alkyl-CONH-alkyl, -alkyl-CON-dialkyl, halo-alkyl-CONH-alkyl, halo-alkyl-CONH-halo-alkyl, alkyl-CONH-halo-dialkyl, -alkyl-hydroxyl, -alkyl-hydroxyl-alkyl, -halo-alkyl-hydroxyl-alkyl, -alkyl-hydroxyl-halo-alkyl, -halo-alkyl-hydroxyl-halo-alkyl, substituted or unsubstituted alkyl-OR¹⁴, substituted or unsubstituted haloalkyl-OR¹⁴, —OR¹⁴, and N(R)R′; or taken together R^{Z} and B₃ may form a substituted or unsubstituted carbocyclic ring or substituted or unsubstituted heterocyclic ring, which may contain one or more heteroatoms and may be saturated or unsaturated;

R is independently selected from the group consisting of hydrogen, cyano, straight chain or branched alkyl, straight chain or branched alkoxy, straight chain or branched halo-alkyl, straight chain or branched halo-alkoxy, alkoxy-alkyl, hydroxyl-alkyl, and carboxy-alkyl; or taken together R and R′ may form a substituted or unsubstituted carbocyclic ring or substituted or unsubstituted or heterocyclic ring, which may contain one or more heteroatoms and may be saturated or unsaturated; or taken together with the N to which they are attached R and R′ may form a moiety selected from the group consisting of substituted straight chain or cyclic guanyl, straight chain or cyclic guanidine, straight chain or cyclic urea, straight chain or cyclic thiourea, straight chain or cyclic carbamate, and straight chain or cyclic thiocarbamate;

R′ is independently selected from the group consisting of cyano, straight chain or branched alkyl, straight chain or branched alkoxy, straight chain or branched halo-alkyl, straight chain or branched halo-alkoxy, alkoxy-alkyl, hydroxyl-alkyl, and carboxy-alkyl; or taken together R and R′ may form a substituted or unsubstituted carbocyclic ring or substituted or unsubstituted or heterocyclic ring, which may contain one or more heteroatoms and may be saturated or unsaturated; or taken together with the N to which they are attached R and R′ may form a moiety selected from the group consisting of substituted straight chain or cyclic guanyl, straight chain or cyclic guanidine, straight chain or cyclic urea, straight chain or cyclic thiourea, straight chain or cyclic carbamate, and straight chain or cyclic thiocarbamate;

Z is independently selected from the group consisting of C or N;

R¹ is a phosphate derivative, a phosphate mimic or a phosphate precursor;

R² and R³ are each independently selected from the group consisting of hydrogen, hydroxyl, halogen, cyano, straight chain or branched alkyl, alkyl-OR⁹, halo-alkyl-OR⁹, alkoxy-OR⁹, alkyl-OC(O)R⁹, halo-alkyl-OC(O)R⁹, alkoxy-OC(O)R⁹, carbocyclic rings, heterocyclic rings which may contain one or more heteroatoms, alkyl-NR⁹R¹⁰, halo-alkyl-NR⁹R¹⁰, and alkoxy-NR⁹R¹⁰, all of which may be optionally substituted with OH, halogen, NHR⁹, NR⁹R¹⁰, straight chain or branched alkoxy, straight chain or branched halo-alkyl, straight chain or branched halo-alkoxy, alkoxy-alkyl, hydroxyl-alkyl, or carboxy-alkyl; or taken together R² and R³ may form a substituted or unsubstituted carbocyclic ring or a substituted or unsubstituted heterocyclic ring, which may contain one or more heteroatoms and may be saturated or unsaturated; or taken together R² and R^{Z} may form a substituted or unsubstituted C₄-C₁₀ fused carbocyclic ring or substituted or unsubstituted C₄-C₁₀ fused heterocyclic rings, which may contain one or more heteroatoms and may be saturated or unsaturated;

R⁹ and R¹⁰ are independently selected from the group consisting of hydrogen, halogen, cyano, straight chain or branched alkyl, straight chain or branched alkoxy, straight chain or branched halo-alkyl, straight chain and branched halo-alkoxy, —C(O)alkyl, —C(O)NH-alkyl, —C(O)N-dialkyl, —C(O)aryl, —C(O)NH-aryl, —C(O)N-alkyl-aryl, —C(O)N-diaryl, —C(O)heteroaryl, —C(O)NH-heteroaryl, —C(O)N-carbocycle, substituted or unsubstituted carbocyclic rings, and substituted or unsubstituted heterocyclic rings, which may contain one or more heteroatoms and may be saturated or unsaturated; or taken together R⁹ and R¹⁰ may form a substituted or unsubstituted carbocyclic ring or a substituted or unsubstituted heterocyclic ring, which may contain one or more heteroatoms and may be saturated or unsaturated;

Y is independently selected from the group consisting of (CR¹¹R¹²)ₙ and (CR¹¹R¹²)ₙNR¹³;

R¹¹, R¹², and R¹³ are independently selected from the group consisting of hydrogen, halogen, cyano, and straight chain or branched alkyl, all of which may be optionally substituted with OH, halogen, straight chain or branched alkoxy, straight chain or branched halo-alkyl, and straight chain and branched halo-alkoxy; or R¹³ may form a 3-8-membered ring together with either R¹¹ or R¹² and the atom to which they are attached;

n is an integer from 0 to 3;

r is an integer from 0 to 7;

E is haloalkyl;

K is selected from the group consisting of

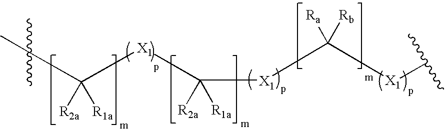

wherein each m is independently selected from an integer between 0 and 6; each p is independently selected from 0 or 1; each X₁ is independently selected from the group consisting of CR¹⁴R¹⁵, NR¹⁴, S, and O, —S(O), —S(O)₂, —OS(O)₂, —OS(O)₂O, —C(O), C(OH), —C(O)O—, a substituted or unsubstituted aromatic, a substituted or unsubstituted heteroaromatic, and any combination thereof, in any orientation; each Rₐ and R_{b} are independently selected from the group consisting of hydrogen, cyano, and straight chain or branched C₁-C₆-alkyl, all of which may be optionally substituted with OH, halogen, e.g., fluoro, straight chain or branched C₁-C₆-alkoxy, straight chain or branched halo-C₁-C₆-alkyl, straight chain and branched halo-C₁-C₆-alkoxy, C₁-C₆-alkoxy-C₁-C₆-alkyl, hydroxyl-C₁-C₆-alkyl, carboxy-C₁-C₆-alkyl, substituted or unsubstituted C₃-C₁₀ carbocyclic rings, and substituted or unsubstituted C₃-C₁₀ heterocyclic rings, which may contain one or more heteroatoms and may be saturated or unsaturated; or each Rₐ and R_{b} may form a 3-8-membered ring together with the carbon to which they are both attached;

R₁ₐ and R₂ₐ are independently selected from the group consisting of hydrogen, halogen, cyano, and straight chain or branched C₁-C₆-alkyl, all of which may be optionally substituted with OH, halogen, straight chain or branched C₁-C₆-alkoxy, straight chain or branched halo-C₁-C₆-alkyl, straight chain and branched halo-C₁-C₆-alkoxy, C₁-C₆-alkoxy-C₁-C₆-alkyl, hydroxyl-C₁-C₆-alkyl, carboxy-C₁-C₆-alkyl, substituted or unsubstituted C₃-C₁₀ carbocyclic rings, and substituted or unsubstituted C₃-C₁₀ heterocyclic rings, which may contain one or more heteroatoms and may be saturated or unsaturated; or each R₁ₐ and R₂ₐ may form a 3-8-membered ring together with the carbon to which they are both attached;

R¹⁴ and R¹⁵ are independently selected from the group consisting of hydrogen, halogen, cyano, straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, and carboxy-C₁-C₆-alkyl; or each R¹⁴ or R¹⁵ may form a 3-8-membered ring together with B₁, SEM, Rₐ, R_{b}, R₁ₐ, or R₂ₐ and the atoms to which they are attached;

R^{S1} through R^{S17} are each independently selected from the group consisting of hydrogen, cyano, halogen, alkyl, halo-alkyl, —OH, —CO—, straight chain or branched alkoxy, straight chain or branched halo-alkyl, straight chain or branched halo-alkoxy, alkoxy-alkyl, hydroxyl-alkyl, -alkyl-hydroxyl, -alkyl-hydroxyl-alkyl, -halo-alkyl-hydroxyl-alkyl, -alkyl-hydroxyl-halo-alkyl, -halo-alkyl-hydroxyl-halo-alkyl, carboxy-alkyl, alkyl-SO₂, alkylcarbonyl, thioether, alkylsulfonyl, alkylcarbonylamino, alkylaminocarbonyl, alkyloxycarbonyl, alkylcarbonyloxy, substituted or unsubstituted aryl, and substituted or unsubstituted heteroaryl, all of which may be optionally substituted with OH, halogen, e.g., fluoro, straight chain or branched alkoxy, straight chain or branched halo-alkyl, straight chain and branched halo-alkoxy, alkoxy-alkyl, hydroxyl-alkyl, carboxy-alkyl, substituted or unsubstituted carbocyclic rings, and substituted or unsubstituted heterocyclic rings, which may contain one or more heteroatoms and may be saturated or unsaturated; and

R^{Z} selected from the group consisting of hydrogen, halogen, cyano, straight chain or branched alkyl, cycloalkyl, straight chain or branched alkoxy, straight chain or branched halo-alkyl, halo-cycloalkyl, straight chain or branched halo-alkoxy, alkoxy-alkyl, hydroxyl-alkyl, alkyl-SO₂, and carboxy-alkyl; or R^{Z} may form a 3-8-membered ring together with B₁, R² or R³ and the atoms to which they are attached. In certain embodiments of Formulae XXVI-XXXIX, the SEM is selected from a group consisting of cyano, straight chain or branched halo-C₁-C₆-alkyl (e.g., CF₃), straight chain or branched halo-C₁-C₆-alkoxy, C₁-C₆-alkoxy-C₁-C₆-alkyl, hydroxyl-C₁-C₆-alkyl, carboxy-C₁-C₆-alkyl, C₁-C₆-alkyl-SO₂ or N(R)R′, wherein R and R′ are each independently hydrogen, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl or C₁-C₆-alkyl-SO₂; alkylcarbonyl, thioether, alkylsulfonyl, alkylcarbonylamino, alkylaminocarbonyl, alkyloxycarbonyl, alkylcarbonyloxy, substituted or unsubstituted aromatic, substituted or unsubstituted heteroaromatic, any straight chain or branched alkylene, straight chain or branched alkenyl, straight chain or branched alkynyl, straight chain or branched alkenylene, arylalkyl, alkylaryl, alkylene-aryl, alkenyl-aryl, alkynyl-aryl or alkenylene-aryl group. In further embodiments the SEM is trifluoromethyl and R^{S2} is C₁-C₆ alkyl. In certain embodiments, the total combination of each p and m is less than or equal to about 21, e.g., less than or equal to about 15, e.g., less than or equal to about 10, e.g., less than or equal to about 8, e.g., less than or equal to about 6. In certain embodiments, r is 3.

In another embodiment, the invention is directed to a compound of Formula XLVIII: 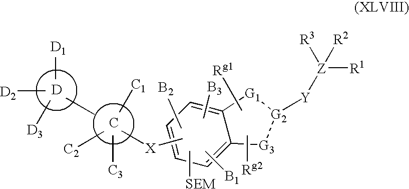
wherein:

the dashed line represents a single or a double bond;

SEM represents a selectivity enhancing moiety;

rings C and D are independently selected from the group consisting of substituted or unsubstituted carbocyclic rings and substituted or unsubstituted heterocyclic rings, which may contain one or more heteroatoms and may be saturated or unsaturated;

G₁ and G₃ are independently selected from the group consisting of O, S, —S(O), —S(O)₂, C(OH), —C(O), CR¹⁴R¹⁵, CR¹⁴, NR¹⁴, and N;

G₂, is selected from the group consisting of C, C(OH), —C(O), CR¹⁴ and N,;

R^{g1} and R^{g2} are each independently selected from the group consisting of hydrogen, cyano, straight chain or branched alkyl, straight chain or branched alkoxy, straight chain or branched halo-alkyl, straight chain or branched halo-alkoxy, alkoxy-alkyl, hydroxyl-alkyl, and carboxy-alkyl;

B₁, B₂, B₃, C₁, C₂, C₃, D₁, D₂, and D₃ are each independently selected from the group consisting of hydrogen, halogen, cyano, straight chain or branched aliphatic, straight chain or branched alkoxy, straight chain or branched halo-alkyl, straight chain or branched halo-alkoxy, alkoxy-alkyl, hydroxyl-alkyl, carboxy-alkyl, alkyl-SO₂ alkylcarbonyl, thioether, alkylsulfonyl, alkylcarbonylamino, alkylaminocarbonyl, alkyloxycarbonyl, alkylcarbonyloxy, substituted or unsubstituted aryl, substituted or unsubstituted heteroaryl, —OH, —C(O)-alkyl, —C(O)-halo-alkyl, —C(O)O-alkyl, —C(O)O-halo-alkyl, —CONH₂, —CONH-alkyl, —CON-dialkyl, —CONH-halo-alkyl, —CON-halo-dialkyl, -alkyl-CONH-alkyl, -alkyl-CON-dialkyl, halo-alkyl-CONH-alkyl, halo-alkyl-CONH-halo-alkyl, alkyl-CONH-halo-dialkyl, -alkyl-hydroxyl, -alkyl-hydroxyl-alkyl, -halo-alkyl-hydroxyl-alkyl, -alkyl-hydroxyl-halo-alkyl, -halo-alkyl-hydroxyl-halo-alkyl, substituted or unsubstituted alkyl-OR¹⁴, substituted or unsubstituted haloalkyl-OR¹⁴, —OR¹⁴, and N(R)R′; or taken together C₂ and D₂ may form a substituted or unsubstituted carbocyclic ring or substituted or unsubstituted heterocyclic ring, which may contain one or more heteroatoms and may be saturated or unsaturated;

R and R′ are each independently selected from the group consisting of hydrogen, cyano, straight chain or branched alkyl, straight chain or branched alkoxy, straight chain or branched halo-alkyl, straight chain or branched halo-alkoxy, alkoxy-alkyl, hydroxyl-alkyl, and carboxy-alkyl; or taken together R and R′ may form a substituted or unsubstituted carbocyclic ring or substituted or unsubstituted or heterocyclic ring, which may contain one or more heteroatoms and may be saturated or unsaturated; or taken together with the N to which they are attached R and R′ may form a moiety selected from the group consisting of substituted straight chain or cyclic guanyl, straight chain or cyclic guanidine, straight chain or cyclic urea, straight chain or cyclic thiourea, straight chain or cyclic carbamate, and straight chain or cyclic thiocarbamate;

Z is independently selected from the group consisting of C or N;

R¹ is a phosphate derivative, a phosphate mimic or a phosphate precursor;

R² and R³ are each independently selected from the group consisting of hydrogen, hydroxyl, halogen, cyano, straight chain or branched alkyl, alkyl-OR⁹, halo-alkyl-OR⁹, alkoxy-OR⁹, alkyl-OC(O)R⁹, halo-alkyl-OC(O)R⁹, alkoxy-OC(O)R⁹, carbocyclic rings, heterocyclic rings which may contain one or more heteroatoms, alkyl-NR⁹R¹⁰, halo-alkyl-NR⁹R¹⁰, and alkoxy-NR⁹R¹⁰, all of which may be optionally substituted with OH, halogen, NHR⁹, NR⁹R¹⁰, straight chain or branched alkoxy, straight chain or branched halo-alkyl, straight chain or branched halo-alkoxy, alkoxy-alkyl, hydroxyl-alkyl, or carboxy-alkyl; or taken together R² and R³ may form a substituted or unsubstituted carbocyclic ring or a substituted or unsubstituted heterocyclic ring, which may contain one or more heteroatoms and may be saturated or unsaturated; or taken together R² and one of R^{g1} or R^{g2} may form a substituted or unsubstituted C₄-C₁₀ fused carbocyclic ring or substituted or unsubstituted C₄-C₁₀ fused heterocyclic rings, which may contain one or more heteroatoms and may be saturated or unsaturated;

R⁹ and R¹⁰ are independently selected from the group consisting of hydrogen, halogen, cyano, straight chain or branched alkyl, straight chain or branched alkoxy, straight chain or branched halo-alkyl, straight chain and branched halo-alkoxy, —C(O)alkyl, —C(O)NH-alkyl, —C(O)N-dialkyl, —C(O)aryl, —C(O)NH-aryl, —C(O)N-alkyl-aryl, —C(O)N-diaryl, —C(O)heteroaryl, —C(O)NH-heteroaryl, —C(O)N-carbocycle, substituted or unsubstituted carbocyclic rings, and substituted or unsubstituted heterocyclic rings, which may contain one or more heteroatoms and may be saturated or unsaturated; or taken together R⁹ and R¹⁰ may form a substituted or unsubstituted carbocyclic ring or a substituted or unsubstituted heterocyclic ring, which may contain one or more heteroatoms and may be saturated or unsaturated; or R⁹ or R¹⁰ together with one of R^{g1} or R^{g2} may form a substituted or unsubstituted C₄-C₁₀ fused carbocyclic ring or substituted or unsubstituted C₄-C₁₀ fused heterocyclic rings, which may contain one or more heteroatoms and may be saturated or unsaturated;

Y is independently selected from the group consisting of (CR¹¹R¹²)ₙ and (CR¹¹R¹²)ₙNR¹³;

R¹¹, R¹², and R¹³ are independently selected from the group consisting of hydrogen, halogen, cyano, and straight chain or branched alkyl, all of which may be optionally substituted with OH, halogen, straight chain or branched alkoxy, straight chain or branched halo-alkyl, and straight chain and branched halo-alkoxy; or R¹³ may form a 3-8-membered ring together with either R¹¹ or R¹² and the atom to which they are attached;

n is an integer from 0 to 3;

X is selected from the group consisting of

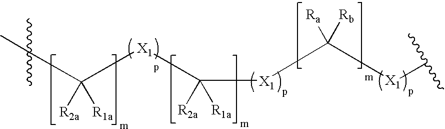

wherein each m is independently selected from an integer between 0 and 6; each p is independently selected from 0 or 1; each XI is independently selected from the group consisting of CR¹⁴R¹⁵, NR¹⁴, S, and O, —S(O), —S(O)₂, —OS(O)₂, —OS(O)₂O—, —C(O), C(OH), —C(O)O—, a substituted or unsubstituted aromatic, a substituted or unsubstituted heteroaromatic, and any combination thereof, in any orientation; each Rₐ and R_{b} are independently selected from the group consisting of hydrogen, cyano, halogen, e.g., F, alkyl, halo-alkyl, —OH, —CO—, straight chain or branched alkoxy, straight chain or branched halo-alkyl, straight chain or branched halo-alkoxy, alkoxy-alkyl, hydroxyl-alkyl, -alkyl-hydroxyl, -alkyl-hydroxyl-alkyl, -halo-alkyl-hydroxyl-alkyl, -alkyl-hydroxyl-halo-alkyl, -halo-alkyl-hydroxyl-halo-alkyl, carboxy-alkyl, alkyl-SO₂, alkylcarbonyl, thioether, alkylsulfonyl, alkylcarbonylamino, alkylaminocarbonyl, alkyloxycarbonyl, alkylcarbonyloxy, substituted or unsubstituted aryl, and substituted or unsubstituted heteroaryl, all of which may be optionally substituted with OH, halogen, e.g., fluoro, straight chain or branched alkoxy, straight chain or branched halo-alkyl, straight chain and branched halo-alkoxy, alkoxy-alkyl, hydroxyl-alkyl, carboxy-alkyl, substituted or unsubstituted carbocyclic rings, and substituted or unsubstituted heterocyclic rings, which may contain one or more heteroatoms and may be saturated or unsaturated; or each Rₐ and R_{b} may form a 3-10-membered ring together with the carbon to which they are both attached;

each R₁ₐ and R₂ₐ are independently selected from the group consisting of hydrogen, cyano, halogen, alkyl, halo-alkyl, —OH, —CO—, straight chain or branched alkoxy, straight chain or branched halo-alkyl, straight chain or branched halo-alkoxy, alkoxy-alkyl, hydroxyl-alkyl, -alkyl-hydroxyl, -alkyl-hydroxyl-alkyl, -halo-alkyl-hydroxyl-alkyl, -alkyl-hydroxyl-halo-alkyl, -halo-alkyl-hydroxyl-halo-alkyl, carboxy-alkyl, alkyl-SO₂, alkylcarbonyl, thioether, alkylsulfonyl, alkylcarbonylamino, alkylaminocarbonyl, alkyloxycarbonyl, alkylcarbonyloxy, substituted or unsubstituted aryl, and substituted or unsubstituted heteroaryl, all of which may be optionally substituted with OH, halogen, e.g., fluoro, straight chain or branched alkoxy, straight chain or branched halo-alkyl, straight chain and branched halo-alkoxy, alkoxy-alkyl, hydroxyl-alkyl, carboxy-alkyl, substituted or unsubstituted carbocyclic rings, and substituted or unsubstituted heterocyclic rings, which may contain one or more heteroatoms and may be saturated or unsaturated; or each R^{1a} and R^{2a} may form a 3-10-membered ring together with the carbon to which they are both attached; and

each R¹⁴ and R¹⁵ is independently selected from the group consisting of hydrogen, halogen, cyano, straight chain or branched alkyl, straight chain or branched alkoxy, straight chain or branched halo-alkyl, straight chain or branched halo-alkoxy, alkoxy-alkyl, hydroxyl-alkyl, alkyl-SO₂, and carboxy-alkyl; or each R¹⁴ or R¹⁵ may form a 3-8-membered ring together with B₁, Rₐ, R_{b}, R₁ₐ, or R₂ₐ and the atoms to which they are attached. In certain embodiments, the total combination of each p and m is less than or equal to about 21, e.g., less than or equal to about 15, e.g., less than or equal to about 10, e..g, less than or equal to about 8, e.g., less than or equal to about 6.

Another embodiment of the invention relates to a compound of Formulae IL-LI: 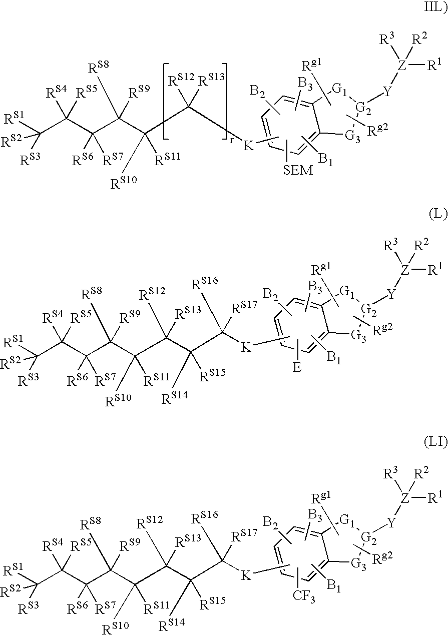
wherein:

the dashed line represents a single or a double bond;

SEM is selected from a group consisting of cyano, straight chain or branched halo-C₁-C₆-alkyl (e.g. CF₃), straight chain or branched halo-C₁-C₆-alkoxy, C₁-C₆-alkoxy-C₁-C₆-alkyl, hydroxyl-C₁-C₆-alkyl, carboxy-C₁-C₆-alkyl, C₁-C₆-alkyl-SO₂ or N(R)R′, wherein R and R′ are each independently hydrogen, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl or C₁-C₆-alkyl-SO₂; alkylcarbonyl, thioether, alkylsulfonyl, alkylcarbonylamino, alkylaminocarbonyl, alkyloxycarbonyl, alkylcarbonyloxy, substituted or unsubstituted aromatic, substituted or unsubstituted heteroaromatic, any straight chain or branched alkylene, straight chain or branched alkenyl, straight chain or branched alkynyl, straight chain or branched alkenylene, arylalkyl, alkylaryl, alkylene-aryl, alkenyl-aryl, alkynyl-aryl or alkenylene-aryl group;

G₁ and G₃ are independently selected from the group consisting of O, S, —S(O), —S(O)₂, C(OH), —C(O), CR¹⁴R¹⁵, CR¹⁴, NR¹⁴, and N;

G₂, is selected from the group consisting of C, C(OH), —C(O), CR¹⁴ and N,;

R^{g1} and R^{g2} are each independently selected from the group consisting of hydrogen, cyano, straight chain or branched alkyl, straight chain or branched alkoxy, straight chain or branched halo-alkyl, straight chain or branched halo-alkoxy, alkoxy-alkyl, hydroxyl-alkyl, and carboxy-alkyl;

B₁, B₂, and B₃ are each independently selected from the group consisting of hydrogen, halogen, cyano, straight chain or branched aliphatic, straight chain or branched alkoxy, straight chain or branched halo-alkyl, straight chain or branched halo-alkoxy, alkoxy-alkyl, hydroxyl-alkyl, carboxy-alkyl, alkyl-SO₂ alkylcarbonyl, thioether, alkylsulfonyl, alkylcarbonylamino, alkylaminocarbonyl, alkyloxycarbonyl, alkylcarbonyloxy, substituted or unsubstituted aryl, substituted or unsubstituted heteroaryl, —OH, —C(O)-alkyl, —C(O)-halo-alkyl, —C(O)O-alkyl, —C(O)O-halo-alkyl, —CONH₂, —CONH-alkyl, —CON-dialkyl, —CONH-halo-alkyl, —CON-halo-dialkyl, —alkyl-CONH-alkyl, -alkyl-CON-dialkyl, halo-alkyl-CONH-alkyl, halo-alkyl-CONH-halo-alkyl, alkyl-CONH-halo-dialkyl, -alkyl-hydroxyl, -alkyl-hydroxyl-alkyl, -halo-alkyl-hydroxyl-alkyl, -alkyl-hydroxyl-halo-alkyl, -halo-alkyl-hydroxyl-halo-alkyl, substituted or unsubstituted alkyl-OR¹⁴, substituted or unsubstituted haloalkyl-OR¹⁴, —OR¹⁴, and N(R)R′;

R is independently selected from the group consisting of hydrogen, cyano, straight chain or branched alkyl, straight chain or branched alkoxy, straight chain or branched halo-alkyl, straight chain or branched halo-alkoxy, alkoxy-alkyl, hydroxyl-alkyl, and carboxy-alkyl; or taken together R and R′ may form a substituted or unsubstituted carbocyclic ring or substituted or unsubstituted or heterocyclic ring, which may contain one or more heteroatoms and may be saturated or unsaturated; or taken together with the N to which they are attached R and R′ may form a moiety selected from the group consisting of substituted straight chain or cyclic guanyl, straight chain or cyclic guanidine, straight chain or cyclic urea, straight chain or cyclic thiourea, straight chain or cyclic carbamate, and straight chain or cyclic thiocarbamate;

R′ is independently selected from the group consisting of cyano, straight chain or branched alkyl, straight chain or branched alkoxy, straight chain or branched halo-alkyl, straight chain or branched halo-alkoxy, alkoxy-alkyl, hydroxyl-alkyl, and carboxy-alkyl; or taken together R and R′ may form a substituted or unsubstituted carbocyclic ring or substituted or unsubstituted or heterocyclic ring, which may contain one or more heteroatoms and may be saturated or unsaturated; or taken together with the N to which they are attached R and R′ may form a moiety selected from the group consisting of substituted straight chain or cyclic guanyl, straight chain or cyclic guanidine, straight chain or cyclic urea, straight chain or cyclic thiourea, straight chain or cyclic carbamate, and straight chain or cyclic thiocarbamate;

Z is independently selected from the group consisting of C or N;

R¹ is a phosphate derivative, a phosphate mimic or a phosphate precursor;

R² and R³ are each independently selected from the group consisting of hydrogen, hydroxyl, halogen, cyano, straight chain or branched alkyl, alkyl-OR⁹, halo-alkyl-OR⁹, alkoxy-OR⁹, alkyl-OC(O)R⁹, halo-alkyl-OC(O)R⁹, alkoxy-OC(O)R⁹, carbocyclic rings, heterocyclic rings which may contain one or more heteroatoms, alkyl-NR⁹R¹⁰, halo-alkyl-NR⁹R¹⁰, and alkoxy-NR⁹R¹⁰, all of which may be optionally substituted with OH, halogen, NHR⁹, NR⁹R¹⁰, straight chain or branched alkoxy, straight chain or branched halo-alkyl, straight chain or branched halo-alkoxy, alkoxy-alkyl, hydroxyl-alkyl, or carboxy-alkyl; or taken together R² and R³ may form a substituted or unsubstituted carbocyclic ring or a substituted or unsubstituted heterocyclic ring, which may contain one or more heteroatoms and may be saturated or unsaturated; or taken together R² and one of R^{g2} or R^{g2} may form a substituted or unsubstituted C₄-C₁₀ fused carbocyclic ring or substituted or unsubstituted C₄-C₁₀ fused heterocyclic rings, which may contain one or more heteroatoms and may be saturated or unsaturated;

R⁹ and R¹⁰ are independently selected from the group consisting of hydrogen, halogen, cyano, straight chain or branched alkyl, straight chain or branched alkoxy, straight chain or branched halo-alkyl, straight chain and branched halo-alkoxy, —C(O)alkyl, —C(O)NH-alkyl, —C(O)N-dialkyl, —C(O)aryl, —C(O)NH-aryl, —C(O)N-alkyl-aryl, —C(O)N-diaryl, —C(O)heteroaryl, —C(O)NH-heteroaryl, —C(O)N-carbocycle, substituted or unsubstituted carbocyclic rings, and substituted or unsubstituted heterocyclic rings, which may contain one or more heteroatoms and may be saturated or unsaturated; or taken together R⁹ and R¹⁰ may form a substituted or unsubstituted carbocyclic ring or a substituted or unsubstituted heterocyclic ring, which may contain one or more heteroatoms and may be saturated or unsaturated;

Y is independently selected from the group consisting of (CR¹¹R²²), and (CR¹¹R¹²)ₙNR¹³;

R¹¹, R¹², and R¹³ are independently selected from the group consisting of hydrogen, halogen, cyano, and straight chain or branched alkyl, all of which may be optionally substituted with OH, halogen, straight chain or branched alkoxy, straight chain or branched halo-alkyl, and straight chain and branched halo-alkoxy; or R¹³ may form a 3-8-membered ring together with either R¹¹ or R¹² and the atom to which they are attached;

n is an integer from 0 to 3;

r is an integer from 0 to 7;

E is haloalkyl;

K is selected from the group consisting of 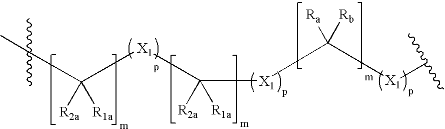
wherein each m is independently selected from an integer between 0 and 6; each p is independently selected from 0 or 1; each XI is independently selected from the group consisting of CR¹⁴R¹⁵, NR¹⁴, S, and O, —S(O), —S(O)₂, —OS(O)₂, —OS(O)₂O—, —C(O), C(OH), —C(O)O—, a substituted or unsubstituted aromatic, a substituted or unsubstituted heteroaromatic, and any combination thereof, in any orientation; each Rₐ and R_{b} are independently selected from the group consisting of hydrogen, cyano, and straight chain or branched C₁-C₆-alkyl, all of which may be optionally substituted with OH, halogen, e.g., fluoro, straight chain or branched C₁-C₆-alkoxy, straight chain or branched halo-C₁-C₆-alkyl, straight chain and branched halo-C₁-C₆-alkoxy, C₁-C₆-alkoxy-C₁-C₆-alkyl, hydroxyl-C₁-C₆-alkyl, carboxy-C₁-C₆-alkyl, substituted or unsubstituted C₃-C₁₀ carbocyclic rings, and substituted or unsubstituted C₃-C₁₀ heterocyclic rings, which may contain one or more heteroatoms and may be saturated or unsaturated; or each Rₐ and R_{b} may form a 3-8-membered ring together with the carbon to which they are both attached;

R₁ₐ and R₂ₐ are independently selected from the group consisting of hydrogen, halogen, cyano, and straight chain or branched C₁-C₆-alkyl, all of which may be optionally substituted with OH, halogen, straight chain or branched C₁-C₆-alkoxy, straight chain or branched halo-C₁-C₆-alkyl, straight chain and branched halo-C₁-C₆-alkoxy, C₁-C₆-alkoxy-C₁-C₆-alkyl, hydroxyl-C₁-C₆-alkyl, carboxy-C₁-C₆-alkyl, substituted or unsubstituted C₃-C₁₀ carbocyclic rings, and substituted or unsubstituted C₃-C₁₀ heterocyclic rings, which may contain one or more heteroatoms and may be saturated or unsaturated; or each R₁ₐ and R₂ₐ may form a 3-8-membered ring together with the carbon to which they are both attached;

R¹⁴ and R¹⁵ are independently selected from the group consisting of hydrogen, halogen, cyano, straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, and carboxy-C₁-C₆-alkyl; or each R¹⁴ or R¹⁵ may form a 3-8-membered ring together with B₁, SEM, Rₐ, R_{b}, R₁ₐ, or R₂ₐ and the atoms to which they are attached; and

R^{S1} through R^{S17} are each independently selected from the group consisting of hydrogen, cyano, halogen, alkyl, halo-alkyl, —OH, —CO—, straight chain or branched alkoxy, straight chain or branched halo-alkyl, straight chain or branched halo-alkoxy, alkoxy-alkyl, hydroxyl-alkyl, -alkyl-hydroxyl, -alkyl-hydroxyl-alkyl, -halo-alkyl-hydroxyl-alkyl, -alkyl-hydroxyl-halo-alkyl, -halo-alkyl-hydroxyl-halo-alkyl, carboxy-alkyl, alkyl-SO₂, alkylcarbonyl, thioether, alkylsulfonyl, alkylcarbonylamino, alkylaminocarbonyl, alkyloxycarbonyl, alkylcarbonyloxy, substituted or unsubstituted aryl, and substituted or unsubstituted heteroaryl, all of which may be optionally substituted with OH, halogen, e.g., fluoro, straight chain or branched alkoxy, straight chain or branched halo-alkyl, straight chain and branched halo-alkoxy, alkoxy-alkyl, hydroxyl-alkyl, carboxy-alkyl, substituted or unsubstituted carbocyclic rings, and substituted or unsubstituted heterocyclic rings, which may contain one or more heteroatoms and may be saturated or unsaturated. In certain embodiments, the SEM is trifluoromethyl and R^{S2} is C₁-C₆ alkyl. In certain embodiments, r is 3.

In another embodiment, the invention relates to compounds of Formulae LII-LVII: 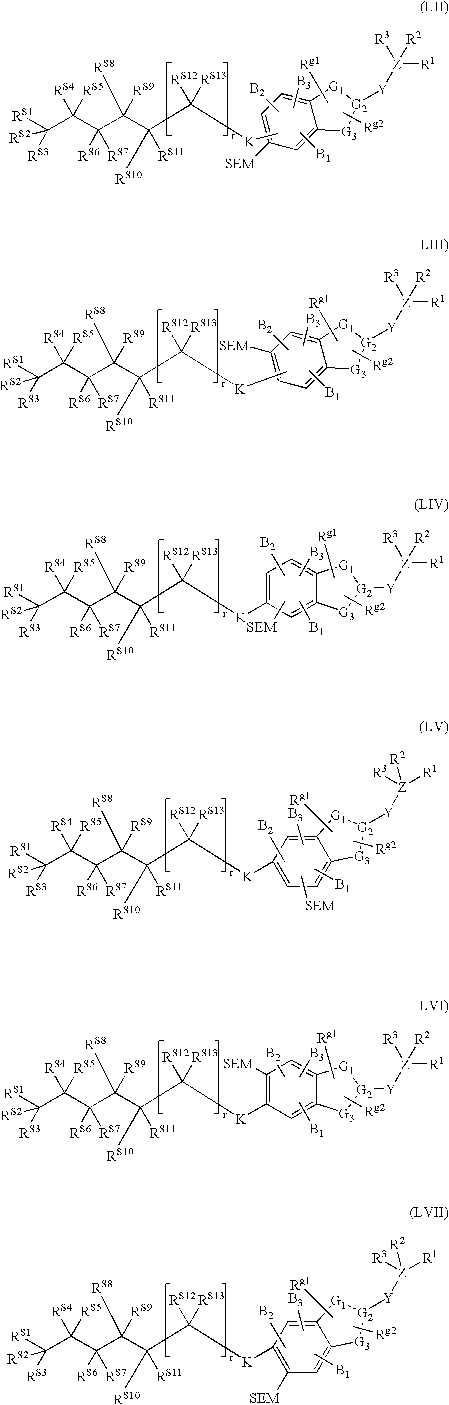
wherein:

the dashed line represents a single or a double bond;

SEM is selected from a group consisting of halogen (erg, Br), cyano, straight chain or branched C₁-C₆-alkyl, straight chain or branched C₁-C₆-alkoxy, straight chain or branched halo-C₁-C₆-alkyl (erg., CF₃), straight chain or branched halo-C₁-C₆-alkoxy, C₁-C₆-alkoxy-C₁-C₆-alkyl, hydroxyl-C₁-C₆-alkyl, carboxy-C₁-C₆-alkyl, C₁-C₆-alkyl-SO₂ or N(R)R′, wherein R and R′ are each independently hydrogen, straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl or C₁-C₆-alkyl-SO₂; alkylcarbonyl, thioether, alkylsulfonyl, alkylcarbonylamino, alkylaminocarbonyl, alkyloxycarbonyl, alkylcarbonyloxy, substituted or unsubstituted aromatic, substituted or unsubstituted heteroaromatic, any straight chain or branched alkylene, straight chain or branched alkenyl, straight chain or branched alkynyl, straight chain or branched alkenylene, arylalkyl, alkylaryl, alkylene-aryl, alkenyl-aryl, alkynyl-aryl or alkenylene-aryl group;

G₁ and G₃ are independently selected from the group consisting of O, S, —S(O), —S(O)₂, C(OH), —C(O), CR¹⁴R¹⁵, CR¹⁴, NR¹⁴, and N;

G₂, is selected from the group consisting of C, C(OH), —C(O), CR¹⁴ and N,;

R^{g1} and R^{g2} are each independently selected from the group consisting of hydrogen, cyano, straight chain or branched alkyl, straight chain or branched alkoxy, straight chain or branched halo-alkyl, straight chain or branched halo-alkoxy, alkoxy-alkyl, hydroxyl-alkyl, and carboxy-alkyl;

B₁, B₂, and B₃ are each independently selected from the group consisting of hydrogen, halogen, cyano, straight chain or branched aliphatic, straight chain or branched alkoxy, straight chain or branched halo-alkyl, straight chain or branched halo-alkoxy, alkoxy-alkyl, hydroxyl-alkyl, carboxy-alkyl, alkyl-SO₂ alkylcarbonyl, thioether, alkylsulfonyl, alkylcarbonylamino, alkylaminocarbonyl, alkyloxycarbonyl, alkylcarbonyloxy, substituted or unsubstituted aryl, substituted or unsubstituted heteroaryl, —OH, —C(O)-alkyl, —C(O)-halo-alkyl, —C(O)O-alkyl, —C(O)O-halo-alkyl, —CONH₂, —CONH-alkyl, —CON-dialkyl, —CONH-halo-alkyl, —CON-halo-dialkyl, -alkyl-CONH-alkyl, -alkyl-CON-dialkyl, halo-alkyl-CONH-alkyl, halo-alkyl-CONH-halo-alkyl, alkyl-CONH-halo-dialkyl, -alkyl-hydroxyl, -alkyl-hydroxyl-alkyl, -halo-alkyl-hydroxyl-alkyl, -alkyl-hydroxyl-halo-alkyl, -halo-alkyl-hydroxyl-halo-alkyl, substituted or unsubstituted alkyl-OR¹⁴, substituted or unsubstituted haloalkyl-OR¹⁴, —OR¹⁴, and N(R)R′;

R is independently selected from the group consisting of hydrogen, cyano, straight chain or branched alkyl, straight chain or branched alkoxy, straight chain or branched halo-alkyl, straight chain or branched halo-alkoxy, alkoxy-alkyl, hydroxyl-alkyl, and carboxy-alkyl; or taken together R and R′ may form a substituted or unsubstituted carbocyclic ring or substituted or unsubstituted or heterocyclic ring, which may contain one or more heteroatoms and may be saturated or unsaturated; or taken together with the N to which they are attached R and R′ may form a moiety selected from the group consisting of substituted straight chain or cyclic guanyl, straight chain or cyclic guanidine, straight chain or cyclic urea, straight chain or cyclic thiourea, straight chain or cyclic carbamate, and straight chain or cyclic thiocarbamate;

R′ is independently selected from the group consisting of cyano, straight chain or branched alkyl, straight chain or branched alkoxy, straight chain or branched halo-alkyl, straight chain or branched halo-alkoxy, alkoxy-alkyl, hydroxyl-alkyl, and carboxy-alkyl; or taken together R and R′ may form a substituted or unsubstituted carbocyclic ring or substituted or unsubstituted or heterocyclic ring, which may contain one or more heteroatoms and may be saturated or unsaturated; or taken together with the N to which they are attached R and R′ may form a moiety selected from the group consisting of substituted straight chain or cyclic guanyl, straight chain or cyclic guanidine, straight chain or cyclic urea, straight chain or cyclic thiourea, straight chain or cyclic carbamate, and straight chain or cyclic thiocarbamate;

Z is independently selected from the group consisting of C or N;

R¹ is a phosphate derivative, a phosphate mimic or a phosphate precursor;

R² and R³ are each independently selected from the group consisting of hydrogen, hydroxyl, halogen, cyano, straight chain or branched alkyl, alkyl-OR⁹, halo-alkyl-OR⁹, alkoxy-OR⁹, alkyl-OC(O)R⁹, halo-alkyl-OC(O)R⁹, alkoxy-OC(O)R⁹, carbocyclic rings, heterocyclic rings which may contain one or more heteroatoms, alkyl-NR⁹R¹⁰, halo-alkyl-NR⁹R¹⁰, and alkoxy-NR⁹R¹⁰, all of which may be optionally substituted with OH, halogen, NHR⁹, NR⁹R¹⁰, straight chain or branched alkoxy, straight chain or branched halo-alkyl, straight chain or branched halo-alkoxy, alkoxy-alkyl, hydroxyl-alkyl, or carboxy-alkyl; or taken together R² and R³ may form a substituted or unsubstituted carbocyclic ring or a substituted or unsubstituted heterocyclic ring, which may contain one or more heteroatoms and may be saturated or unsaturated; or taken together R² and one of R^{g2} or R^{g2} may form a substituted or unsubstituted C₄-C₁₀ fused carbocyclic ring or substituted or unsubstituted C₄-C₁₀ fused heterocyclic rings, which may contain one or more heteroatoms and may be saturated or unsaturated;

R⁹ and R¹⁰ are independently selected from the group consisting of hydrogen, halogen, cyano, straight chain or branched alkyl, straight chain or branched alkoxy, straight chain or branched halo-alkyl, straight chain and branched halo-alkoxy, —C(O)alkyl, —C(O)NH-alkyl, —C(O)N-dialkyl, —C(O)aryl, —C(O)NH-aryl, —C(O)N-alkyl-aryl, —C(O)N-diaryl, —C(O)heteroaryl, —C(O)NH-heteroaryl, —C(O)N-carbocycle, substituted or unsubstituted carbocyclic rings, and substituted or unsubstituted heterocyclic rings, which may contain one or more heteroatoms and may be saturated or unsaturated; or taken together R⁹ and R¹⁰ may form a substituted or unsubstituted carbocyclic ring or a substituted or unsubstituted heterocyclic ring, which may contain one or more heteroatoms and may be saturated or unsaturated; or R⁹ or R¹⁰ together with one of R^{g2} or R^{g2} may form a substituted or unsubstituted C₄-C₁₀ fused carbocyclic ring or substituted or unsubstituted C₄-C₁₀ fused heterocyclic rings, which may contain one or more heteroatoms and may be saturated or unsaturated;

Y is independently selected from the group consisting of (CR¹¹R¹²)ₙ and (CR¹¹R¹²)ₙNR¹³;

R¹¹, R¹², and R¹³ are independently selected from the group consisting of hydrogen, halogen, cyano, and straight chain or branched alkyl, all of which may be optionally substituted with OH, halogen, straight chain or branched alkoxy, straight chain or branched halo-alkyl, and straight chain and branched halo-alkoxy; or R¹³ may form a 3-8-membered ring together with either R¹¹ or R¹² and the atom to which they are attached;

n is an integer from 0 to 3;

r is an integer from 0 to 7;

K is selected from the group consisting of

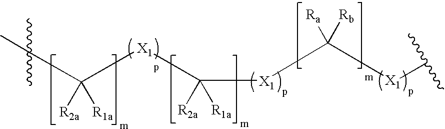

wherein each m is independently selected from an integer between 0 and 6; each p is independently selected from 0 or 1; each X₁ is independently selected from the group consisting of CR¹⁴R¹⁵, NR¹⁴, S, and O, —S(O), —S(O)₂, —OS(O)₂O—, —C(O), C(OH), —C(O)O—, a substituted or unsubstituted aromatic, a substituted or unsubstituted heteroaromatic, and any combination thereof, in any orientation; each Rₐ and R_{b} are independently selected from the group consisting of hydrogen, cyano, and straight chain or branched C₁-C₆-alkyl, all of which may be optionally substituted with OH, halogen, e.g., fluoro, straight chain or branched C₁-C₆-alkoxy, straight chain or branched halo-C₁-C₆-alkyl, straight chain and branched halo-C₁-C₆-alkoxy, C₁-C₆-alkoxy-C₁-C₆-alkyl, hydroxyl-C₁-C₆-alkyl, carboxy-C₁-C₆-alkyl, substituted or unsubstituted C₃-C₁₀ carbocyclic rings, and substituted or unsubstituted C₃-C₁₀ heterocyclic rings, which may contain one or more heteroatoms and may be saturated or unsaturated; or each Rₐ and R_{b} may form a 3-8-membered ring together with the carbon to which they are both attached;

R₁ₐ and R₂ₐ are independently selected from the group consisting of hydrogen, halogen, cyano, and straight chain or branched C₁-C₆-alkyl, all of which may be optionally substituted with OH, halogen, straight chain or branched C₁-C₆-alkoxy, straight chain or branched halo-C₁-C₆-alkyl, straight chain and branched halo-C₁-C₆-alkoxy, C₁-C₆-alkoxy-C₁-C₆-alkyl, hydroxyl-C₁-C₆-alkyl, carboxy-C₁-C₆-alkyl, substituted or unsubstituted C₃-C₁₀ carbocyclic rings, and substituted or unsubstituted C₃-C₁₀ heterocyclic rings, which may contain one or more heteroatoms and may be saturated or unsaturated; or each R₁ₐ and R₂ₐ may form a 3-8-membered ring together with the carbon to which they are both attached;

R¹⁴ and R¹⁵ are independently selected from the group consisting of hydrogen, halogen, cyano, straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, and carboxy-C₁-C₆-alkyl; or each R¹⁴ or R¹⁵ may form a 3-8-membered ring together with B₁, SEM, Rₐ, R_{b}, R₁ₐ, or R₂ₐ and the atoms to which they are attached; and

R^{S1} through R^{S17} are each independently selected from the group consisting of hydrogen, cyano, halogen, alkyl, halo-alkyl, —OH, —CO—, straight chain or branched alkoxy, straight chain or branched halo-alkyl, straight chain or branched halo-alkoxy, alkoxy-alkyl, hydroxyl-alkyl, -alkyl-hydroxyl, -alkyl-hydroxyl-alkyl, -halo-alkyl-hydroxyl-alkyl, -alkyl-hydroxyl-halo-alkyl, -halo-alkyl-hydroxyl-halo-alkyl, carboxy-alkyl, alkyl-SO₂, alkylcarbonyl, thioether, alkylsulfonyl, alkylcarbonylamino, alkylaminocarbonyl, alkyloxycarbonyl, alkylcarbonyloxy, substituted or unsubstituted aryl, and substituted or unsubstituted heteroaryl, all of which may be optionally substituted with OH, halogen, e.g., fluoro, straight chain or branched alkoxy, straight chain or branched halo-alkyl, straight chain and branched halo-alkoxy, alkoxy-alkyl, hydroxyl-alkyl, carboxy-alkyl, substituted or unsubstituted carbocyclic rings, and substituted or unsubstituted heterocyclic rings, which may contain one or more heteroatoms and may be saturated or unsaturated. In certain embodiments of Formulae LII-LVII, the SEM is selected from a group consisting of cyano, straight chain or branched C₁-C₆-alkyl, straight chain or-branched C₁-C₆-alkoxy, straight chain or branched halo-C₁-C₆-alkyl (e.g., CF₃), straight chain or branched halo-C₁-C₆-alkoxy, C₁-C₆-alkoxy-C₁-C₆-alkyl, hydroxyl-C₁-C₆-alkyl, carboxy-C₁-C₆-alkyl, C₁-C₆-alkyl-SO₂ or N(R)R′, wherein R and R′ are each independently hydrogen, straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl or C₁-C₆-alkyl-SO₂; alkylcarbonyl, thioether, alkylsulfonyl, alkylcarbonylamino, alkylaminocarbonyl, alkyloxycarbonyl, alkylcarbonyloxy, substituted or unsubstituted aromatic, substituted or unsubstituted heteroaromatic, any straight chain or branched alkylene, straight chain or branched alkenyl, straight chain or branched alkynyl, straight chain or branched alkenylene, arylalkyl, alkylaryl, alkylene-aryl, alkenyl-aryl, alkynyl-aryl or alkenylene-aryl group. In particular embodiments, the SEM is trifluoromethyl and R⁵² is C₁-C₆ alkyl. In certain embodiments, r is 3.

In certain embodiments of the compounds of the invention, R¹ is L₁-O—H or L₁-O-L₂, wherein L₁ is a linking moiety and L₂ is a labile moiety. In particular embodiments, R₁ is selected from the group consisting of -alkyl-OH, -halo-alkyl-OH, alkoxy-OH, -alkyl-OCOR⁴, -halo-alkyl-OCOR⁴, -alkoxy-OCOR⁴, -alkyl-OC(O)NR⁴R⁵, -halo-alkyl-OC(O)NHR⁴ R⁵, -alkoxy-OC(O)NR⁴R⁵, —(CH₂)_{q}CO₂R⁶, and —(CH₂)ₙCH₂═CHC(O)OR⁶, wherein

q is an integer between 0 and 4;

R⁴ and R⁵ are independently selected from the group consisting of hydrogen, straight chain or branched C₁-C₆-alkyl, all of which may be optionally substituted with OH, halogen, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl, substituted or unsubstituted C₃-C₁₀ carbocyclic rings, and substituted or unsubstituted C₃-C₁₀ heterocyclic rings, which may contain one or more heteroatoms and may be saturated or unsaturated; and

R⁶ is selected from the group consisting of hydrogen, straight chain or branched C₁-C₆-alkyl, straight chain or branched halo-C₁-C₆-alkyl, substituted or unsubstituted aryl group, and a prodrug derivatizing moiety (PDM).

In certain embodiments of the compounds of the invention, R₁ is selected from the group consisting of —(CH₂)_{q} OPO₂R⁷R⁸, —(CH₂)_{q}OPO₃R⁷R⁸, and —(CH₂)_{q}OPO₂(S)R⁷R⁸, wherein

q is an integer between 0 and 4; and

R⁷ and R⁸ are each independently selected from the group consisting of hydrogen, straight chain or branched C₁-C₆-alkyl, straight chain or branched halo-C₁-C₆-alkyl, substituted or unsubstituted aryl group, and a prodrug derivatizing moiety (PDM).

In certain embodiments of the compounds of the invention, R¹ is —L₁-Z₂, wherein L₁ is a linking moiety and Z₂ is a non-hydrolyzable moiety covalently bonded to L₁. In a particular embodiment, R₁ is selected from the group consisting of —(CH₂)_{q}CH₂PO₃R⁷R⁸, and —(CH₂)_{q}C(Y₁)(Y₂)PO₃R⁷R⁸, wherein

q is an integer between 0 and 4;

Y₁ and Y₂ are independently selected from the group consisting of hydrogen, straight chain or branched C₁-C₆-alkyl, all of which may be optionally substituted with OH, halogen, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl, substituted or unsubstituted C₃-C₁₀ carbocyclic rings, and substituted or unsubstituted C₃-C₁₀ heterocyclic rings, which may contain one or more heteroatoms and may be saturated or unsaturated; and

R⁷ and R⁸ are each independently selected from the group consisting of hydrogen, straight chain or branched C₁-C₆-alkyl, straight chain or branched halo-C₁-C₆-alkyl, substituted or unsubstituted aryl group, and a prodrug derivatizing moiety (PDM).

In certain embodiments of the compounds of the invention, the PDM is selected from the group consisting of:

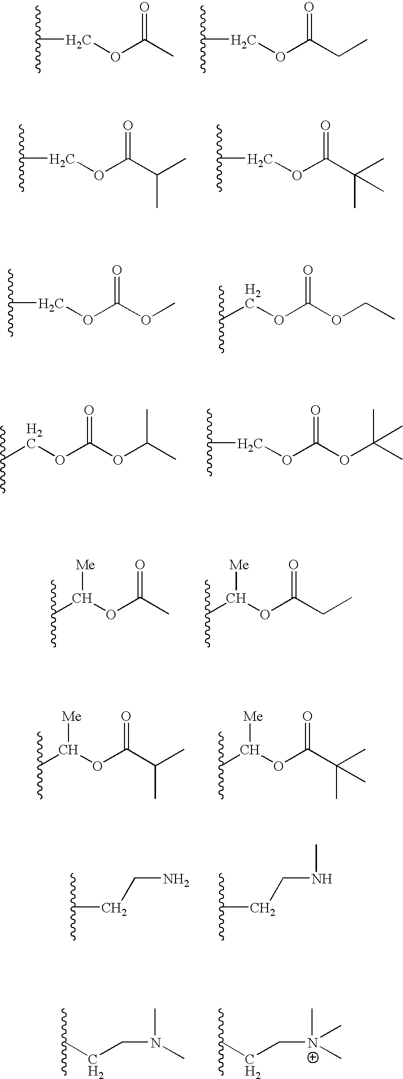

In certain embodiments of the compounds of the invention, taken together R² and R³ form a substituted or unsubstituted C₃-C₁₀ carbocyclic ring or a substituted or unsubstituted C₁-C₁₀ heterocyclic ring, which may contain one or more heteroatoms and may be saturated or unsaturated, said ring contains at least one halogen.

In certain embodiments of the compounds of the invention, each of A, B, C, D is independently selected from the group consisting of an aromatic ring and a heteroaromatic ring.

In certain embodiments of the compounds of the invention, X is independently selected from the group consisting of straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl, C₁-C₆-alkyl-SO₂ alkylcarbonyl, thioether, alkylsulfonyl, alkylcarbonylamino, alkylaminocarbonyl, alkyloxycarbonyl, alkylcarbonyloxy, substituted or unsubstituted aromatic, and substituted or unsubstituted heteroaromatic; or taken together with either B₂ or C₁, R¹⁵ may form a substituted or unsubstituted C₃-C₁₀ carbocyclic ring or substituted or unsubstituted C₃-C₁₀ heterocyclic ring, which may contain one or more heteroatoms and may be saturated or unsaturated In a particular embodiment, X is selected from the group consisting of —CH₂NR¹⁴—, —CH₂NR¹⁴(CO)—, —CHFNR¹⁴—, —CHFNR¹⁴(CO)—, —CF₂NR¹⁴—, —CF₂NR¹⁴(CO)—, —CH₂(CO)—, —CHF(CO)—, —CF₂(CO)—, —(CO)CH₂—, —(CO)CHF—, —(CO)CF₂—, —CH₂(CHOH)—, —CHF(CHOH)—, —CF₂(CHOH)—, —(CHOH)CH₂—, —(CHOH)CHF—, —(CHOH)CF₂—, —NH(CO)—, —(CO)NH—, —(CO)—, —(CO)₂—, —O—, —S—, —SO—, —SO₂—, —CH₂O—, —CH₂CH₂O—, —CH₂OCH₂—, —OCH₂CH₂—, —OCH₂O—, —CH₂S—, —CH₂SO—, —CH₂SO₂—, —OCH₂—, —SCH₂—, —SOCH₂—, —SO₂CH₂—, —CHFO—, —CHFS—, —CHFSO—, —CHFSO₂—, —OCHF—, —SCHF—, —SOCHF—, —SO₂CHF—, —CF₂O—, —CF₂S—, —CF₂SO—, —CF₂SO₂—, —OCF₂—, —SCF₂—, —SOCF₂—, —SO₂CF₂—, —SO₂CF₂—, —NR¹⁴SO₂—, —SO₂NR¹⁴—, —CF₂—, —CF₂CF₂—, a aromatic group, and a heteroaromatic group.

In certain embodiments of the compounds of the invention, the SEM is selected from a group consisting of cyano, straight chain or branched C₁-C₆-alkyl, straight chain or-branched C₁-C₆-alkoxy, straight chain or branched halo-C₁-C₆-alkyl (e.g., CF₃), straight chain or branched halo-C₁-C₆-alkoxy, C₁-C₆-alkoxy-C₁-C₆-alkyl, hydroxyl-C₁-C₆-alkyl, carboxy-C₁-C₆-alkyl, C₁-C₆-alkyl-SO₂ or N(R)R′, wherein R and R′ are each independently hydrogen, straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl or C₁-C₆-alkyl-SO₂; alkylcarbonyl, thioether, alkylsulfonyl, alkylcarbonylamino, alkylaminocarbonyl, alkyloxycarbonyl, alkylcarbonyloxy, substituted or unsubstituted aromatic, substituted or unsubstituted heteroaromatic, any straight chain or branched alkylene, straight chain or branched alkenyl, straight chain or branched alkynyl, straight chain or branched alkenylene, arylalkyl, alkylaryl, alkylene-aryl, alkenyl-aryl, alkynyl-aryl or alkenylene-aryl group.

In certain embodiments of the compounds of the invention, the SEM is selected from a group consisting of cyano, straight chain or branched halo-C₁-C₆-alkyl (e.g, CF₃), straight chain or branched halo-C₁-C₆-alkoxy, C₁-C₆-alkoxy-C₁-C₆-alkyl, hydroxyl-C₁-C₆-alkyl, carboxy-C₁-C₆-alkyl, C₁-C₆-alkyl-SO₂ or N(R)R′, wherein R and R′ are each independently hydrogen, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl or C₁-C₆-alkyl-SO₂; alkylcarbonyl, thioether, alkylsulfonyl, alkylcarbonylamino, alkylaminocarbonyl, alkyloxycarbonyl, alkylcarbonyloxy, substituted or unsubstituted aromatic, substituted or unsubstituted heteroaromatic, any straight chain or branched alkylene, straight chain or branched alkenyl, straight chain or branched alkynyl, straight chain or branched alkenylene, arylalkyl, alkylaryl, alkylene-aryl, alkenyl-aryl, alkynyl-aryl or alkenylene-aryl group. In further embodiments the SEM is trifluoromethyl and R^{S2} is C₁-C₆ alkyl.

Moreover, it should be understood that the compounds of the present invention, e.g., compounds of any of Formulae I-XLVII, comprise compounds that satisfy valency requirements known to the ordinarily skilled artisan. Additionally, compounds of the present invention comprise stable compounds as well as though compounds that may be modified, e.g., chemically or through appropriate formulation, to become stable. In certain embodiments, such stability is guided by time periods that are sufficient to allow administration to and/or treatment of a subject.

Particular compounds of the invention include, but are not limited to, those set forth below and salts thereof. While the compounds below may be represented as alcohols (e.g., R⁶ of Formula I is hydroxy) or phosphates (e.g., R⁶ of Formula I is —OPO₃H₂), specific compounds of the invention further include phosphate mimics, phosphate derivatives, and phosphate precursors of these compounds including, but not limited to, for example, carboxylate, methylenephosphonate, thiophosphate hydroxymethylenephosphonate, and fluoromethylenephosphonate.

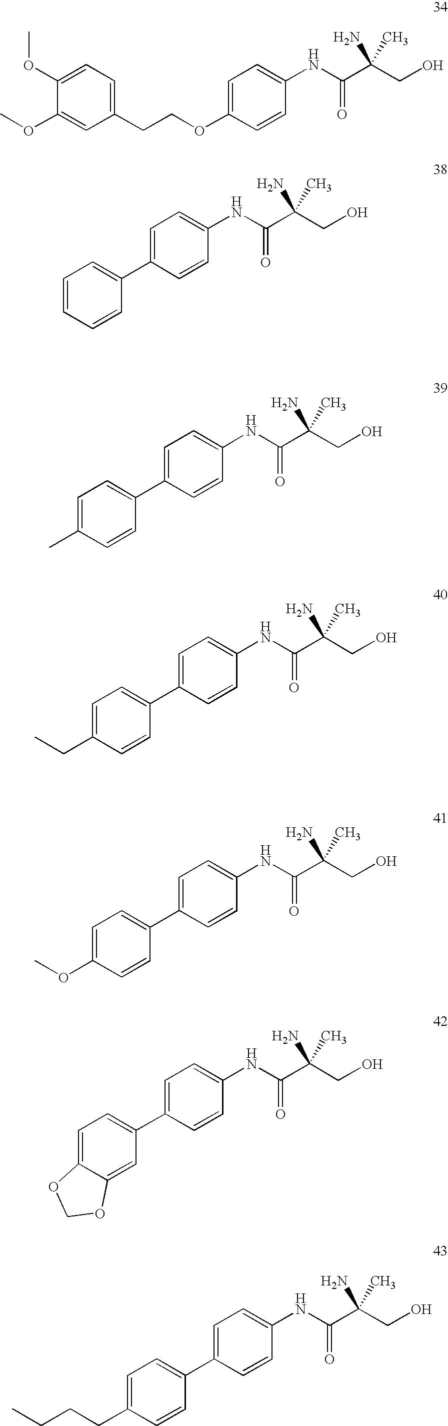

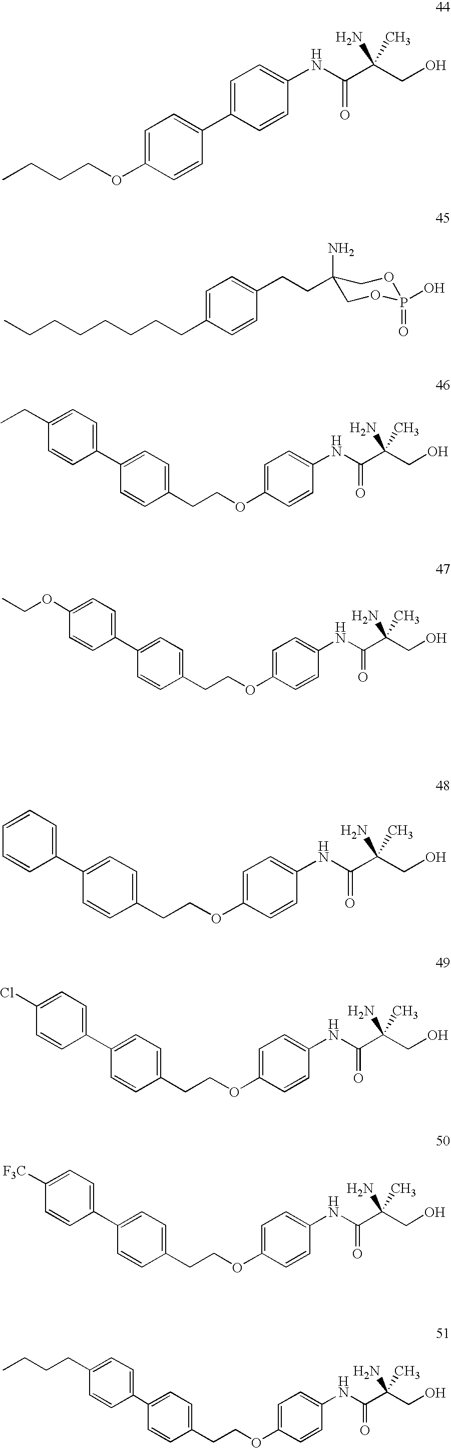

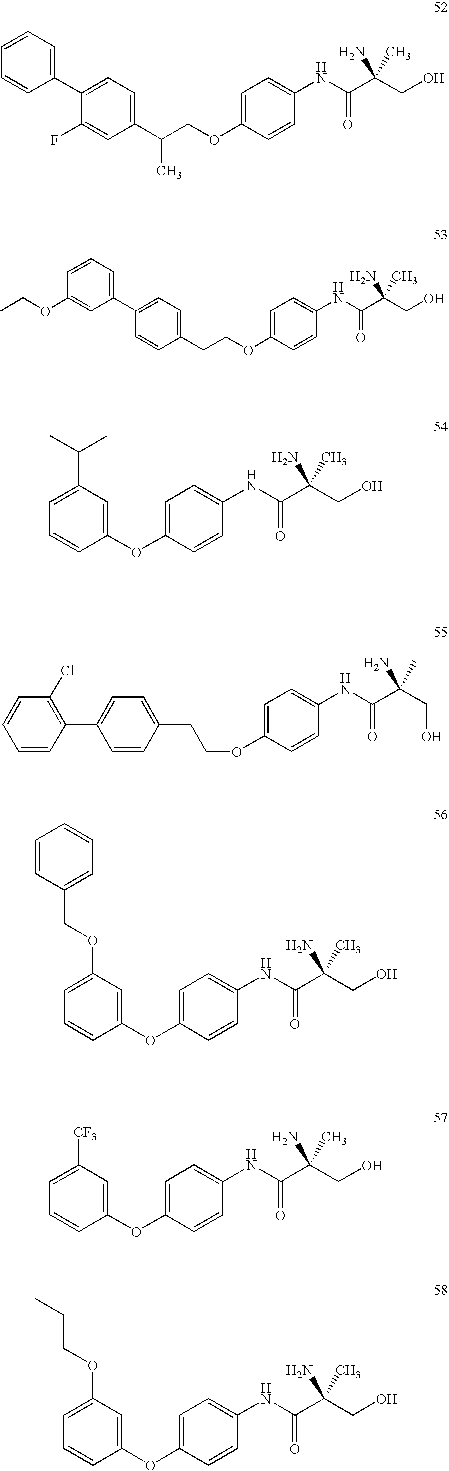

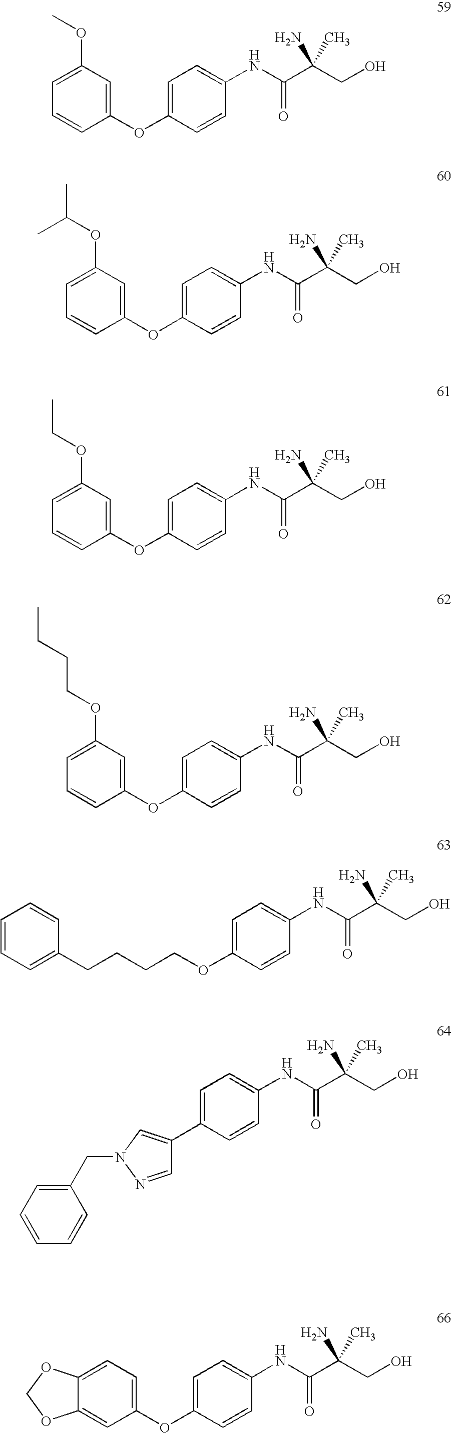

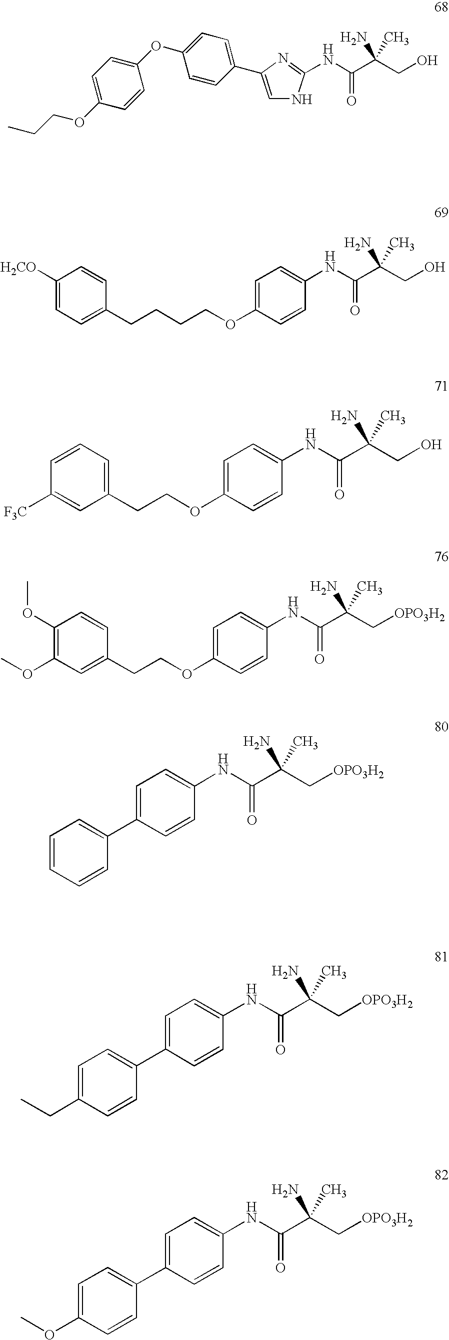

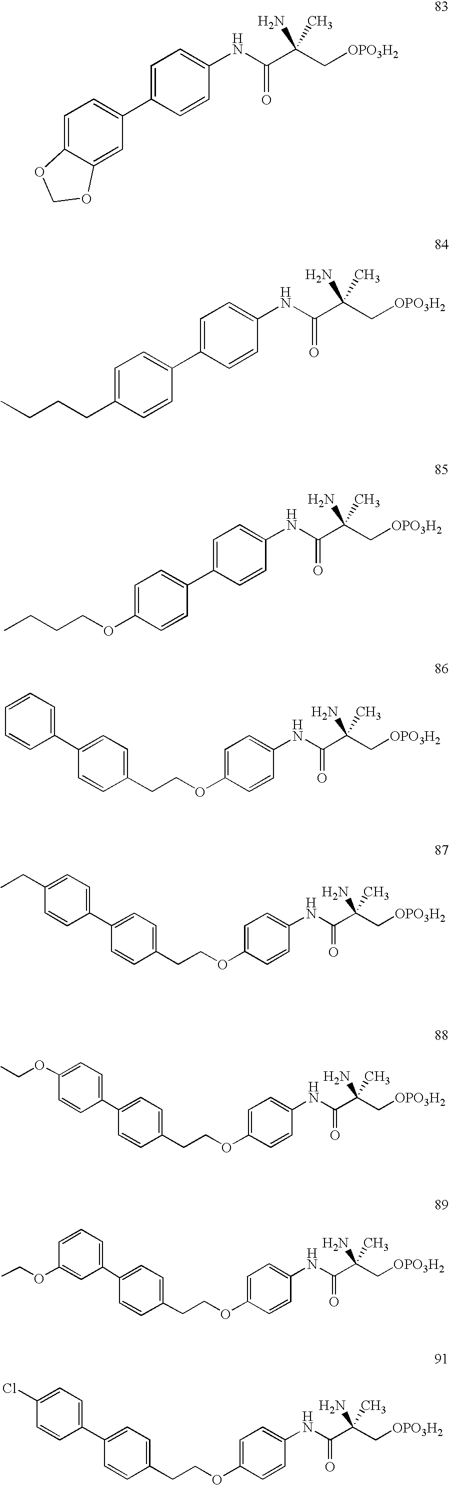

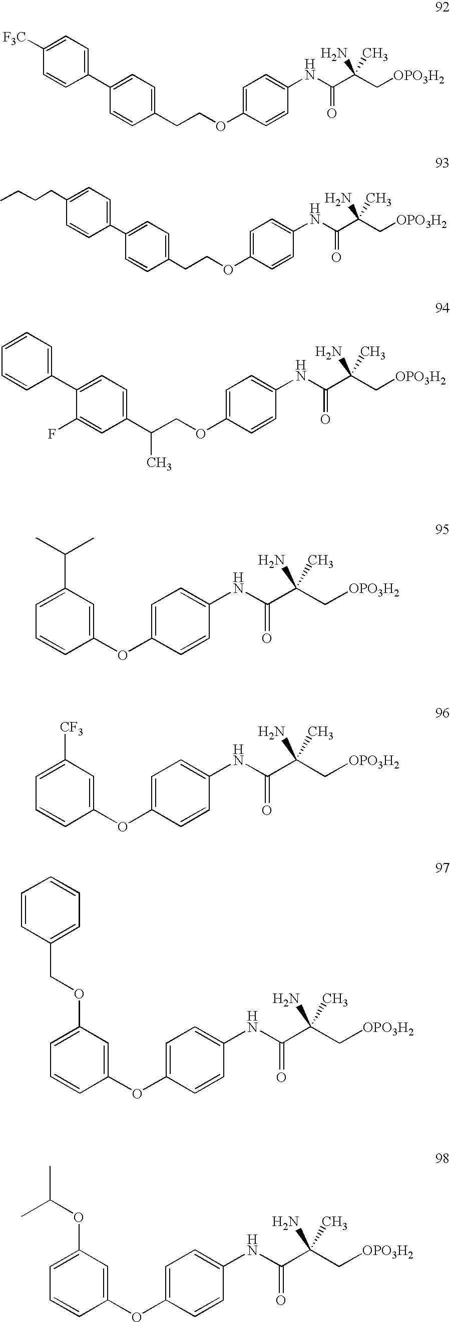

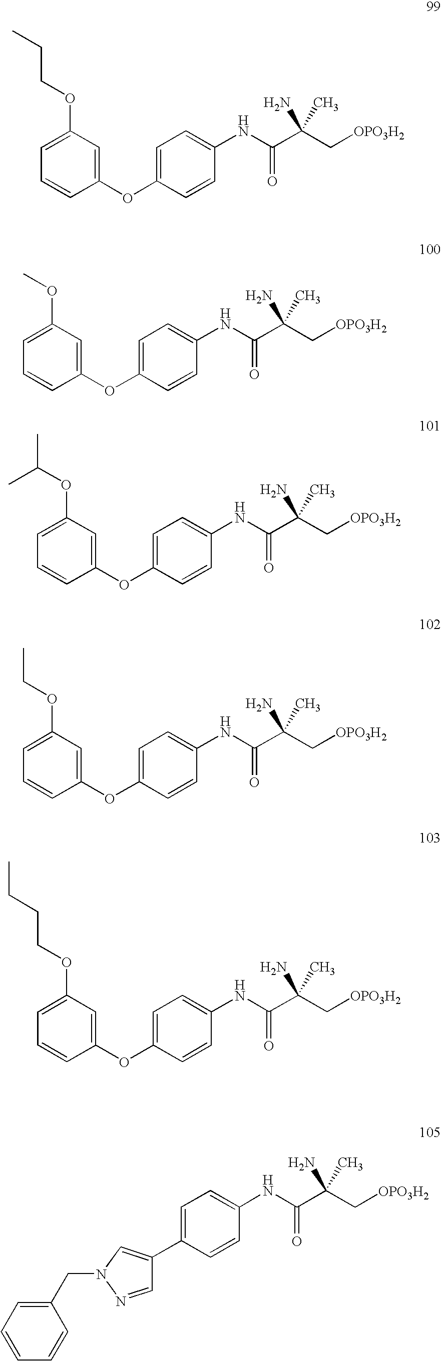

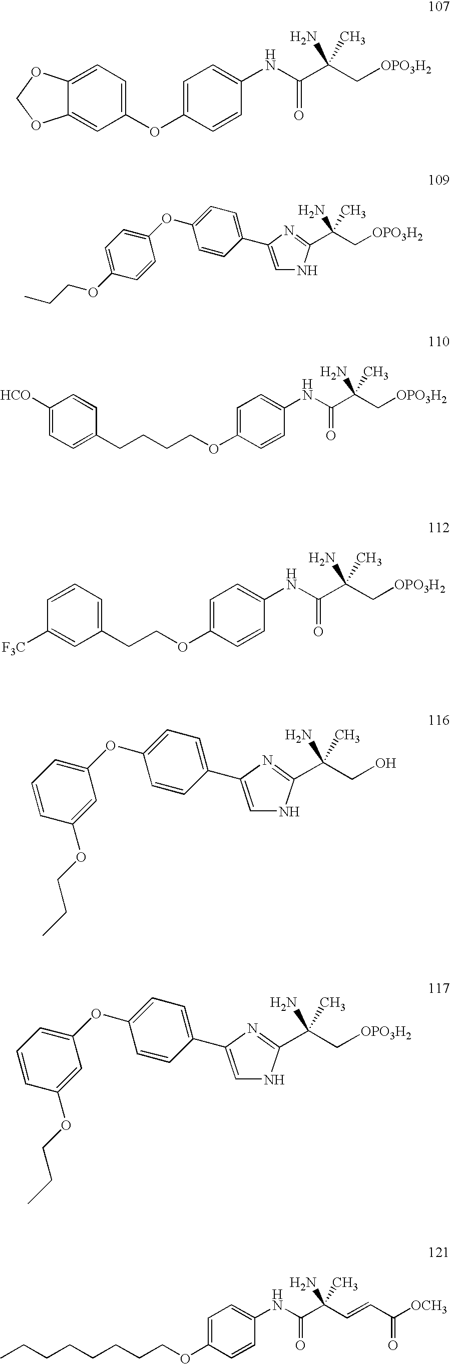

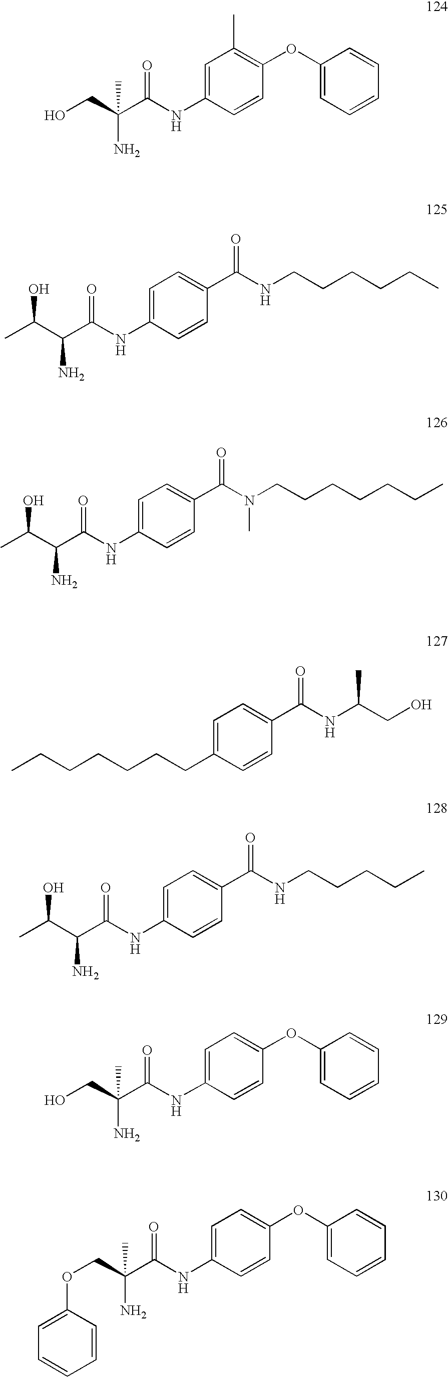

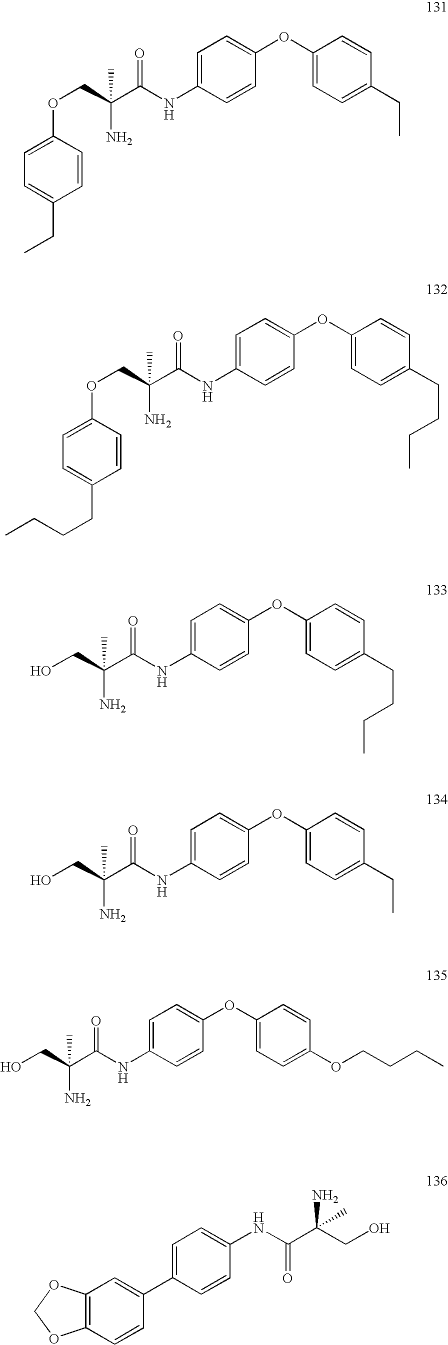

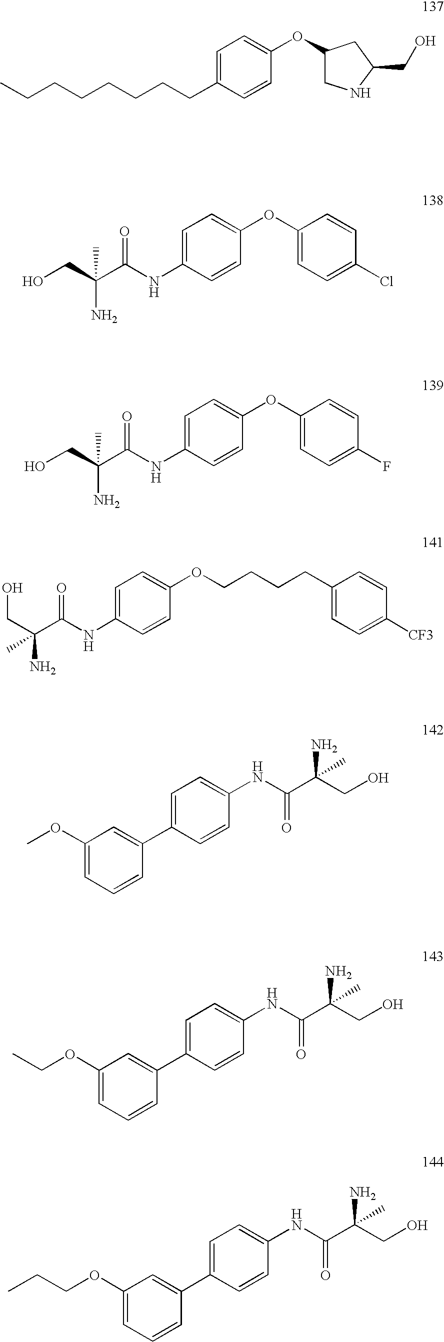

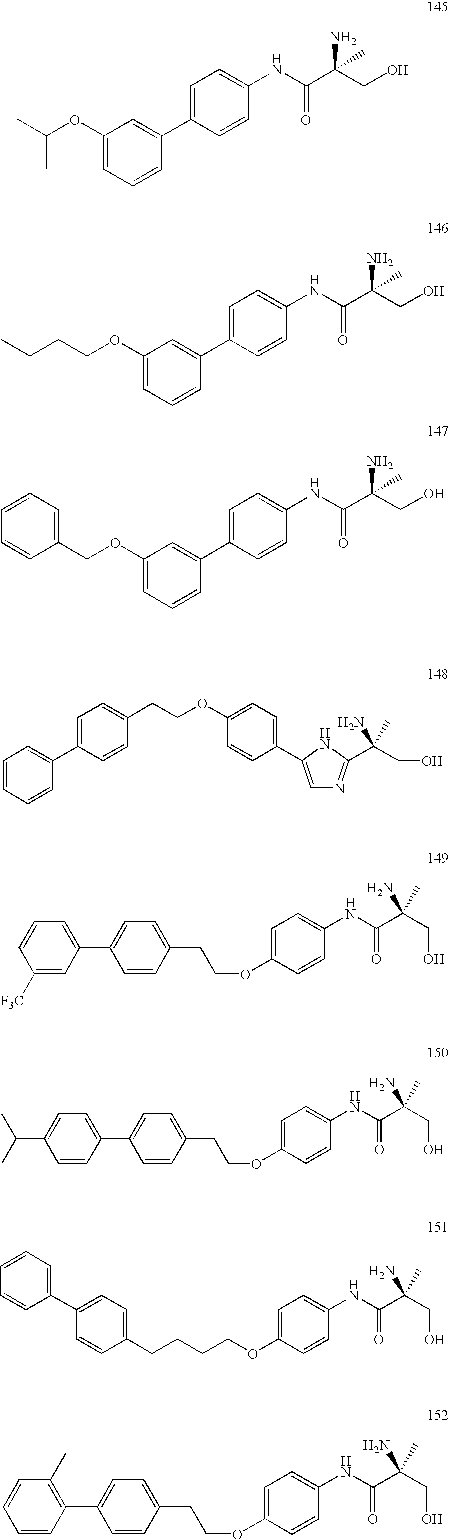

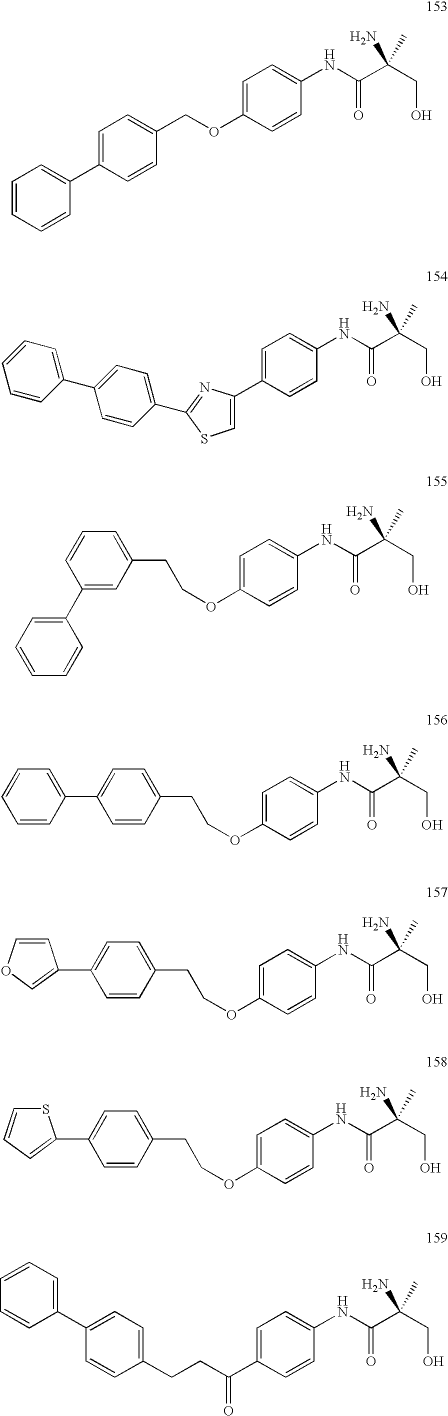

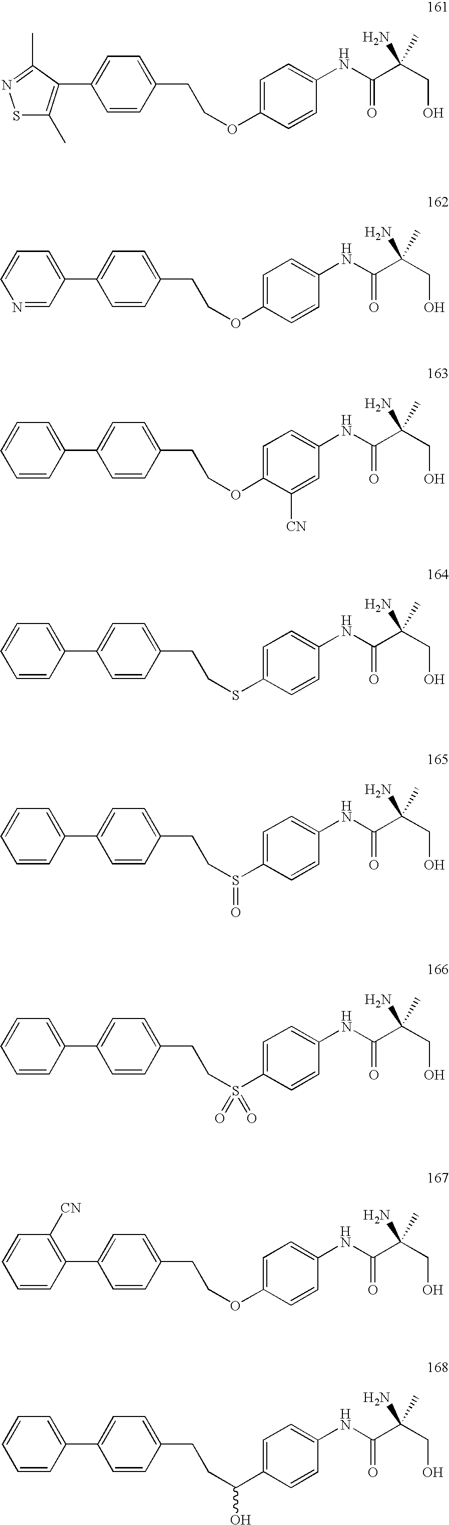

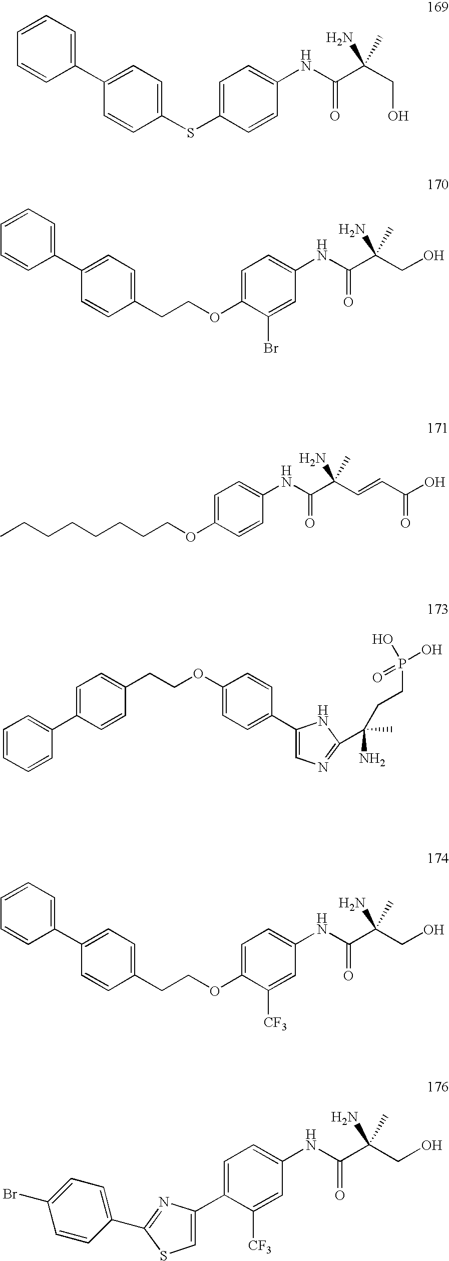

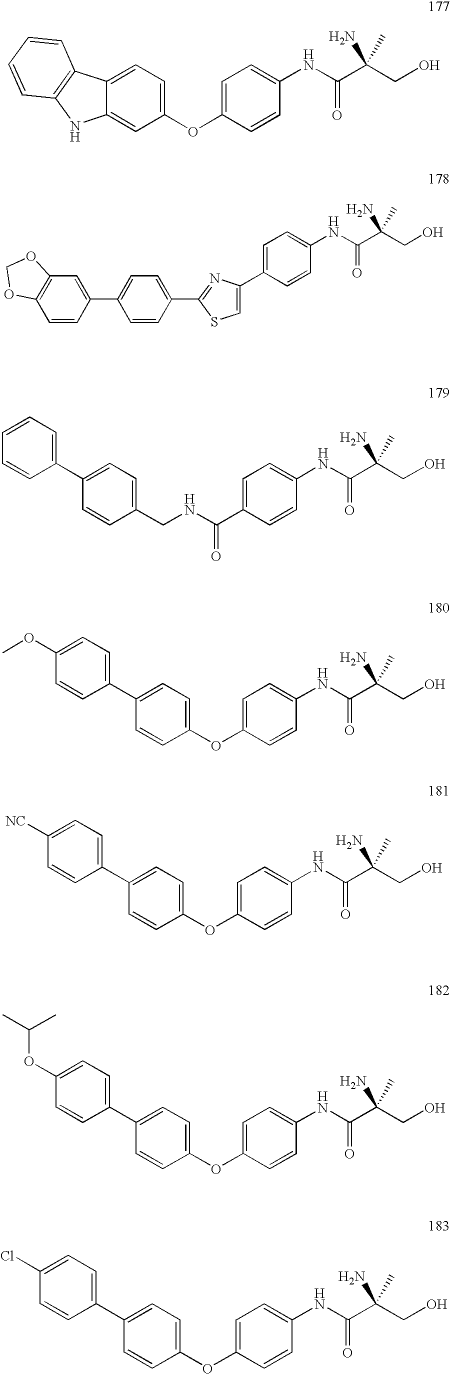

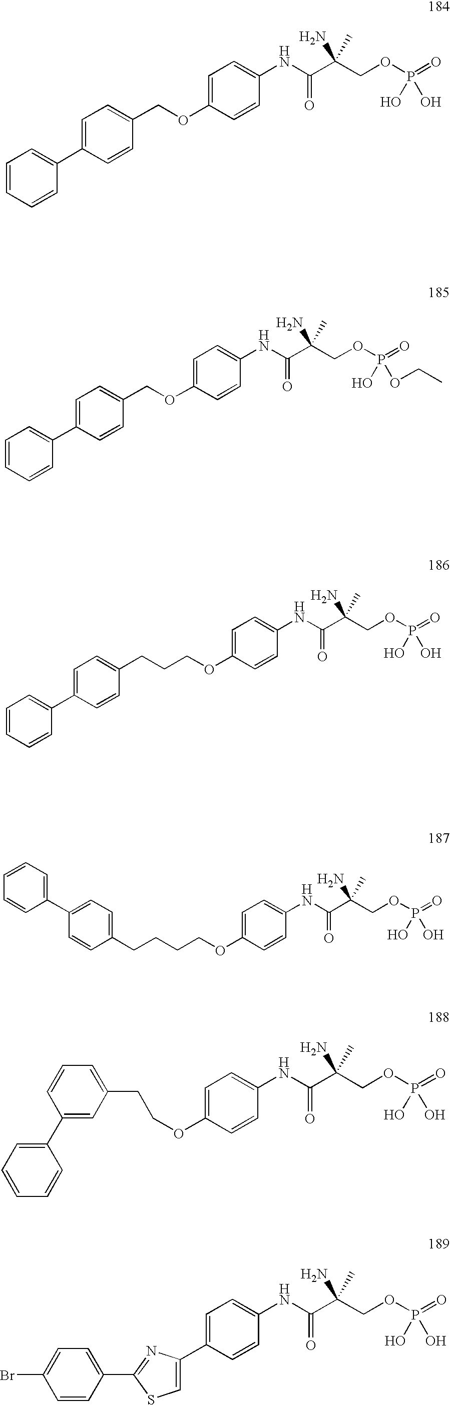

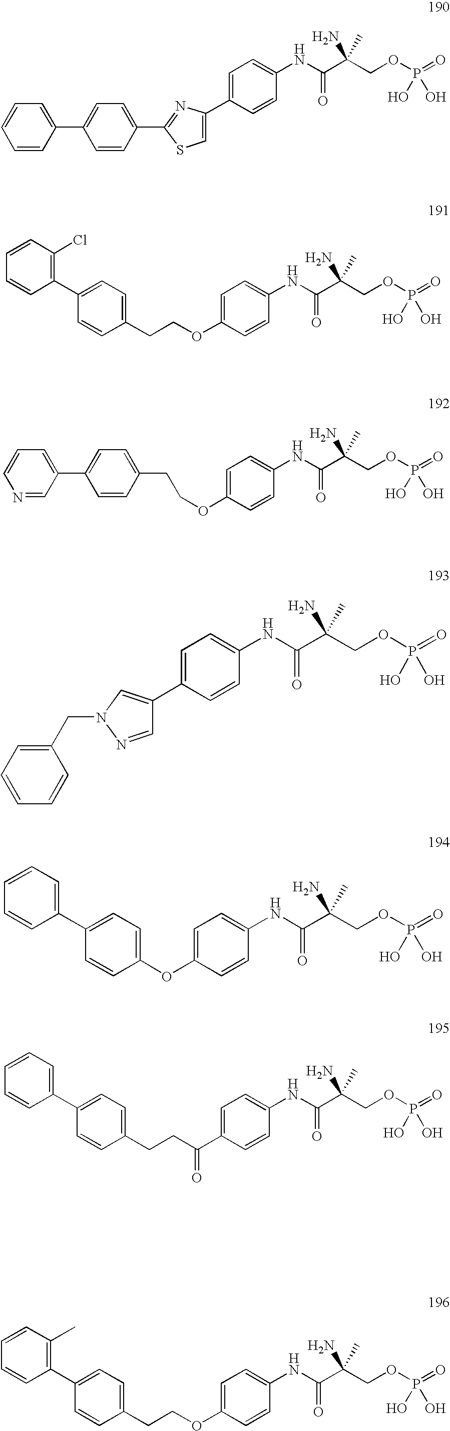

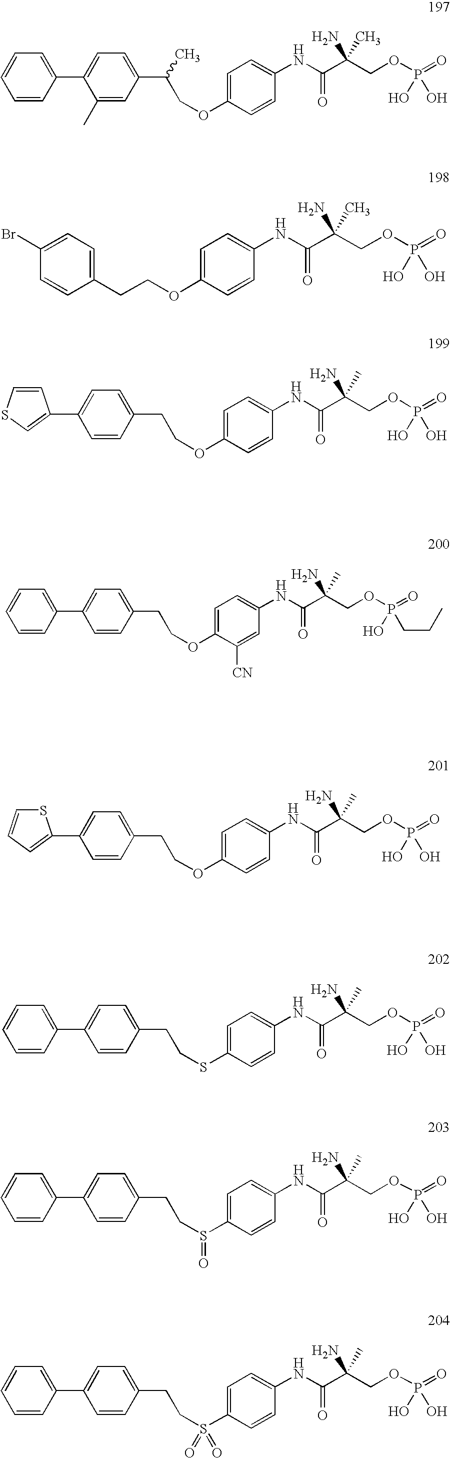

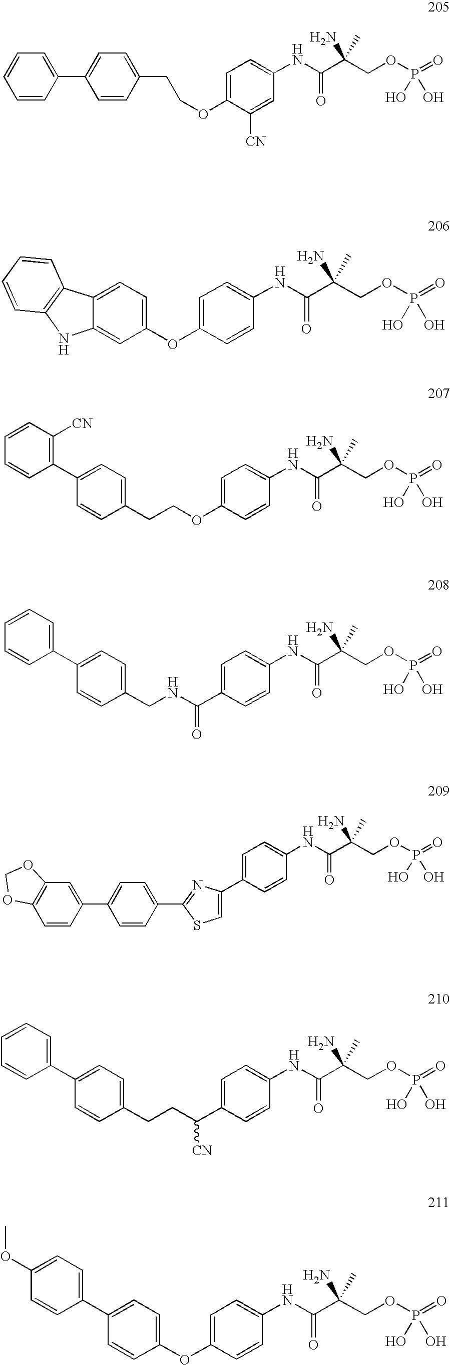

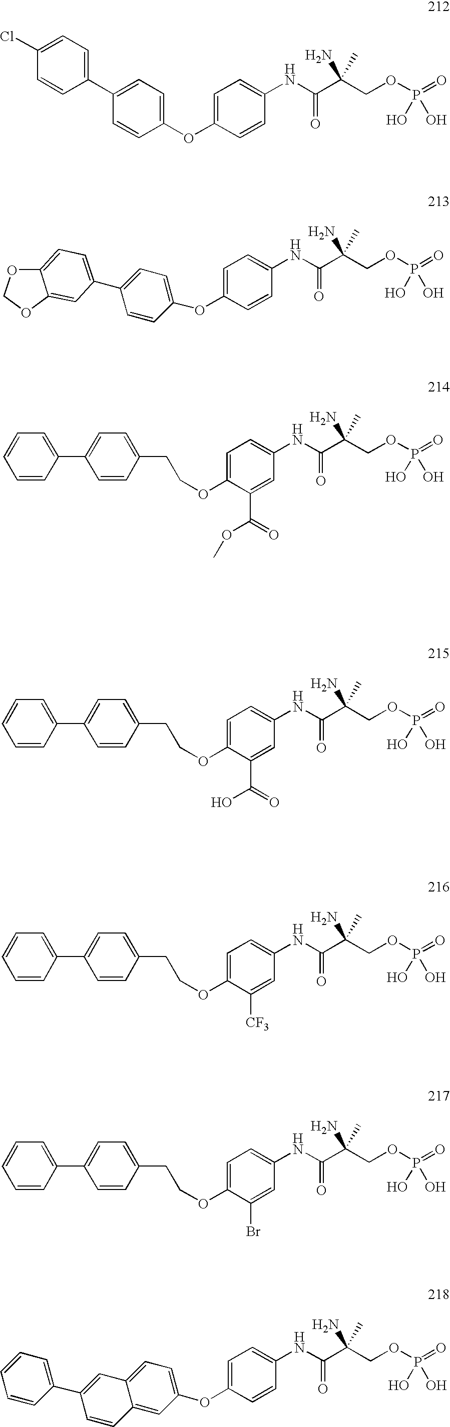

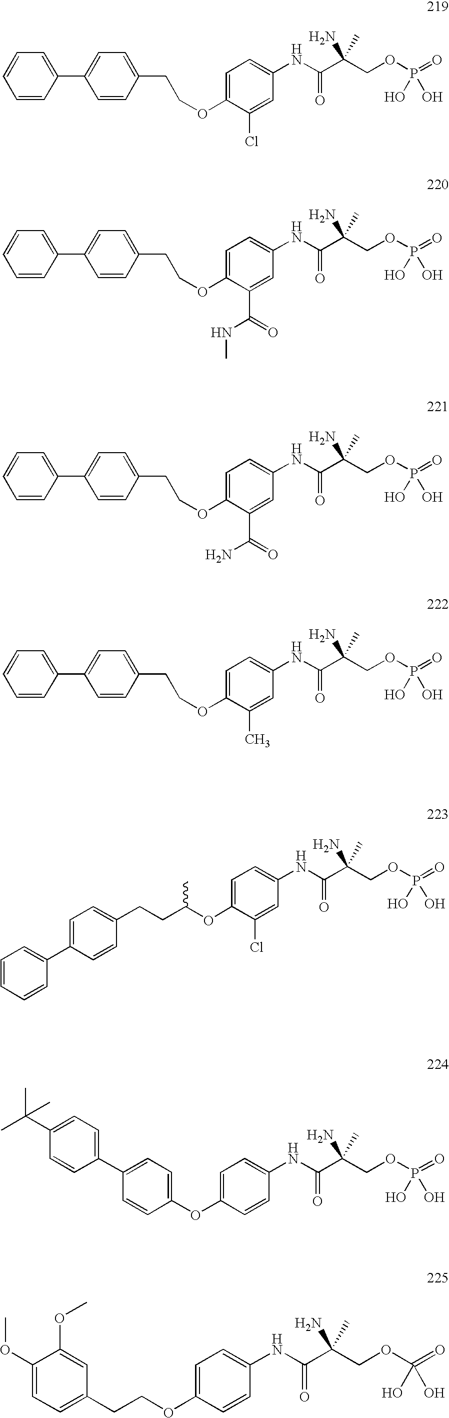

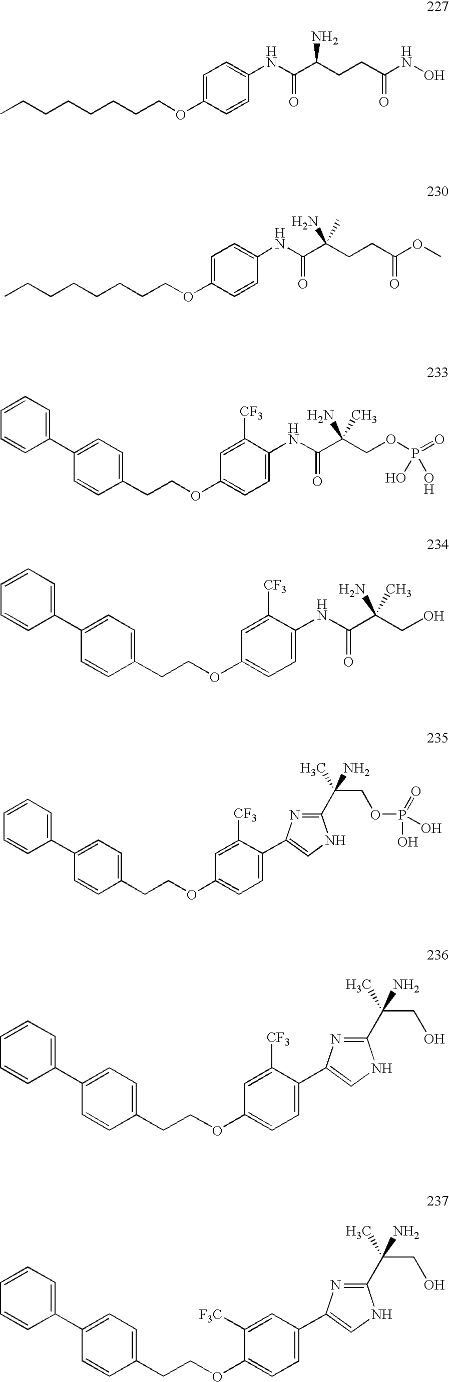

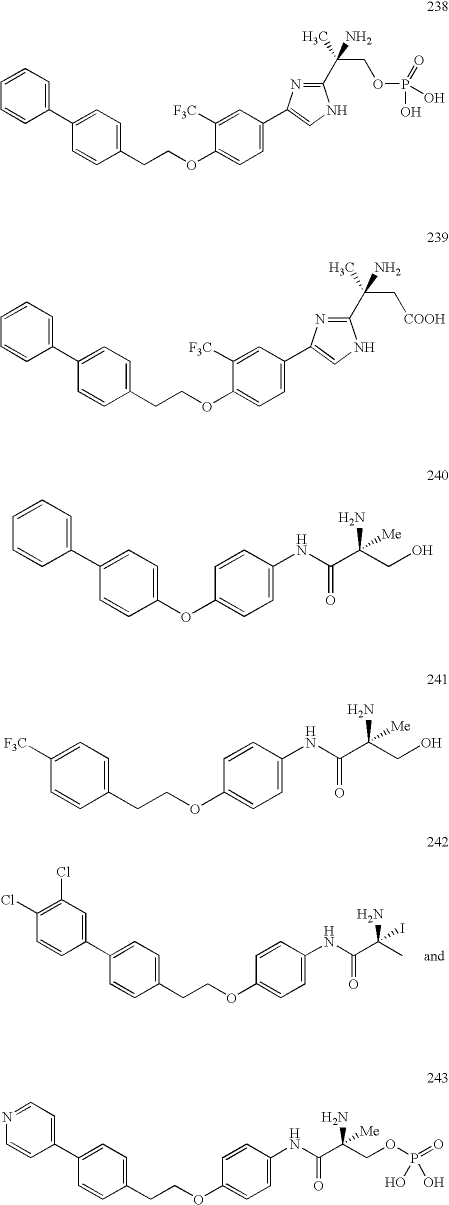

In addition, compounds of the invention include the following compounds:

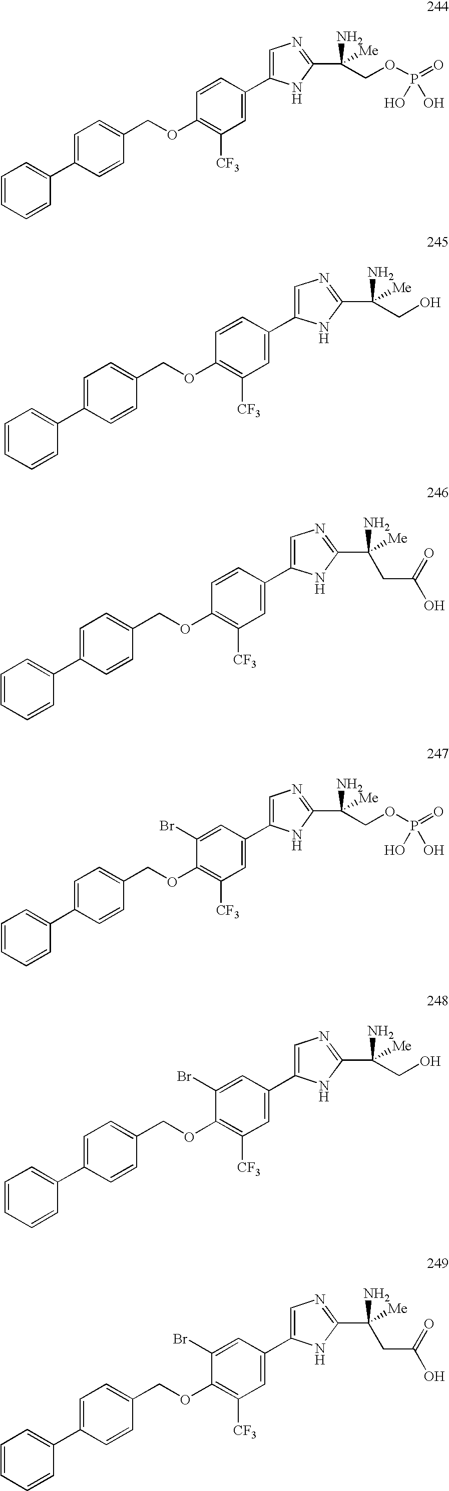

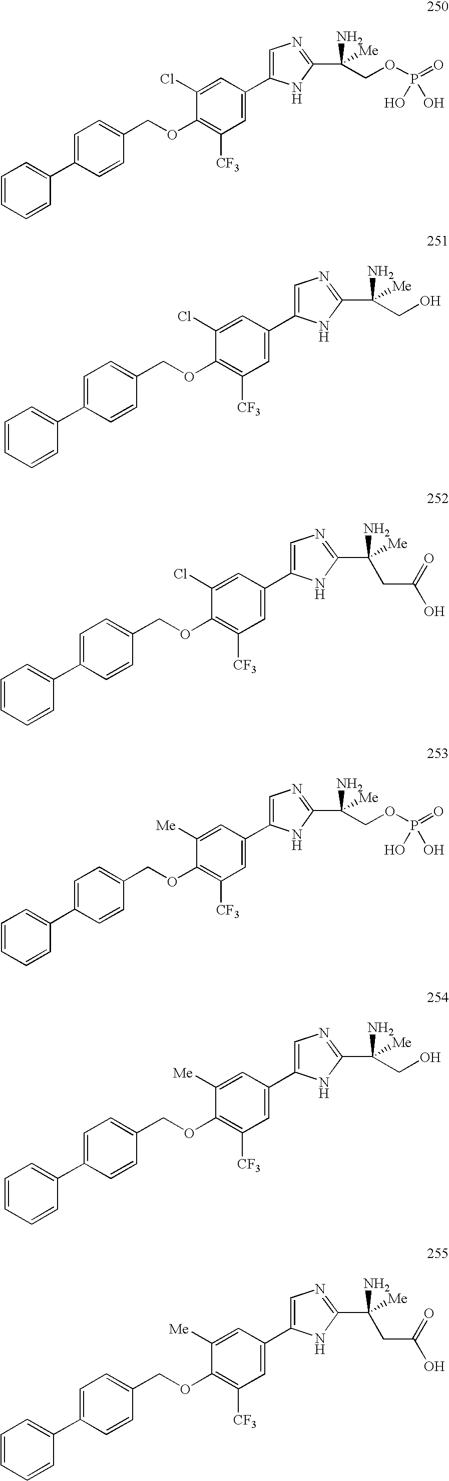

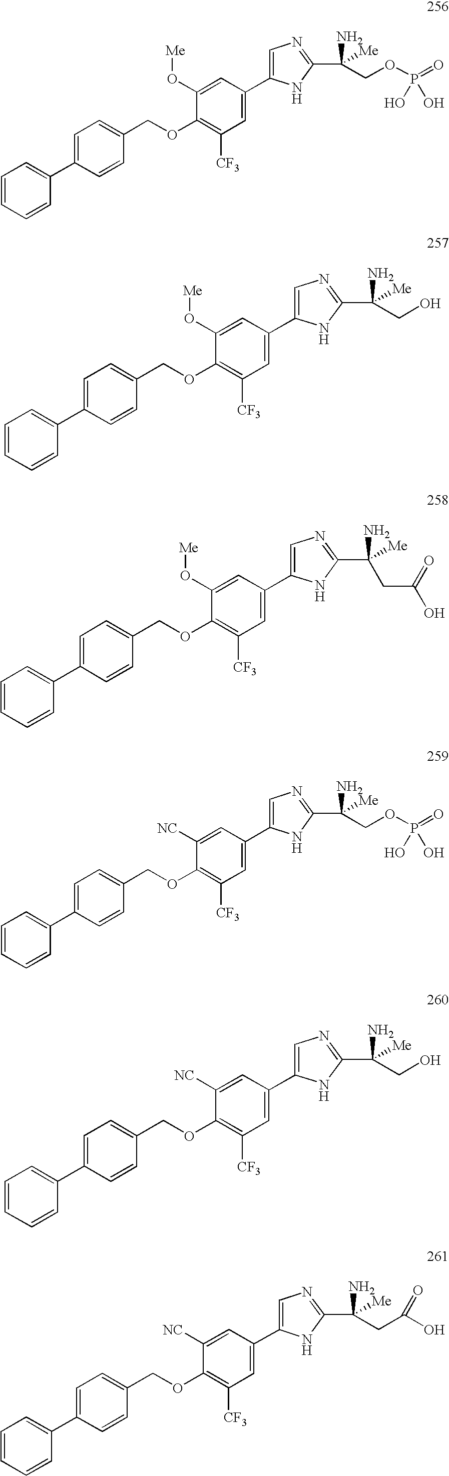

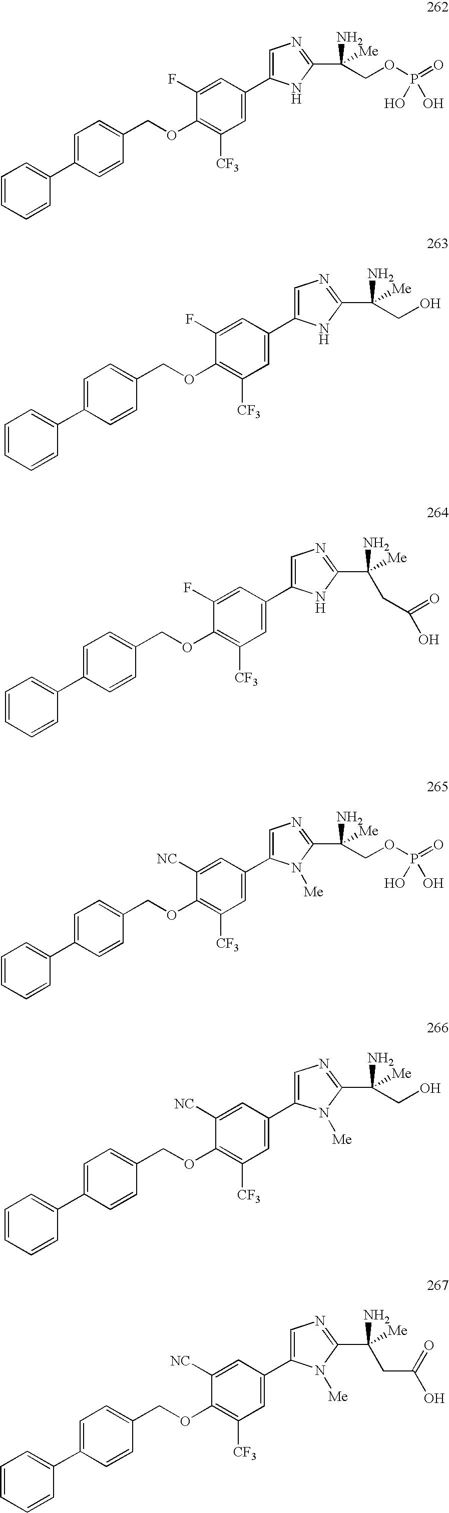

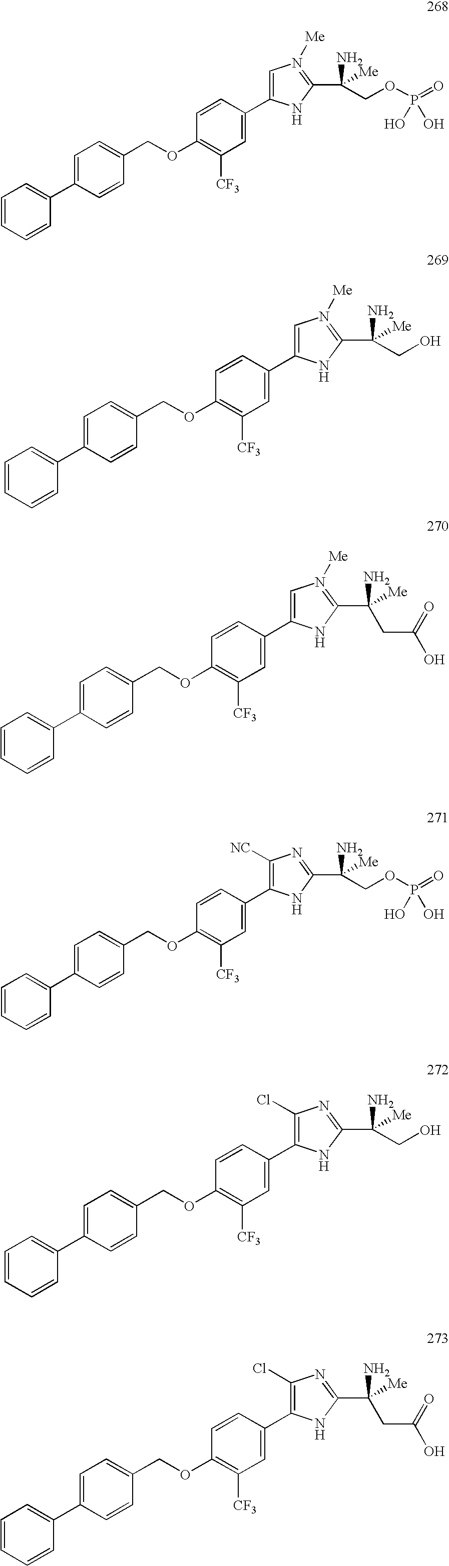

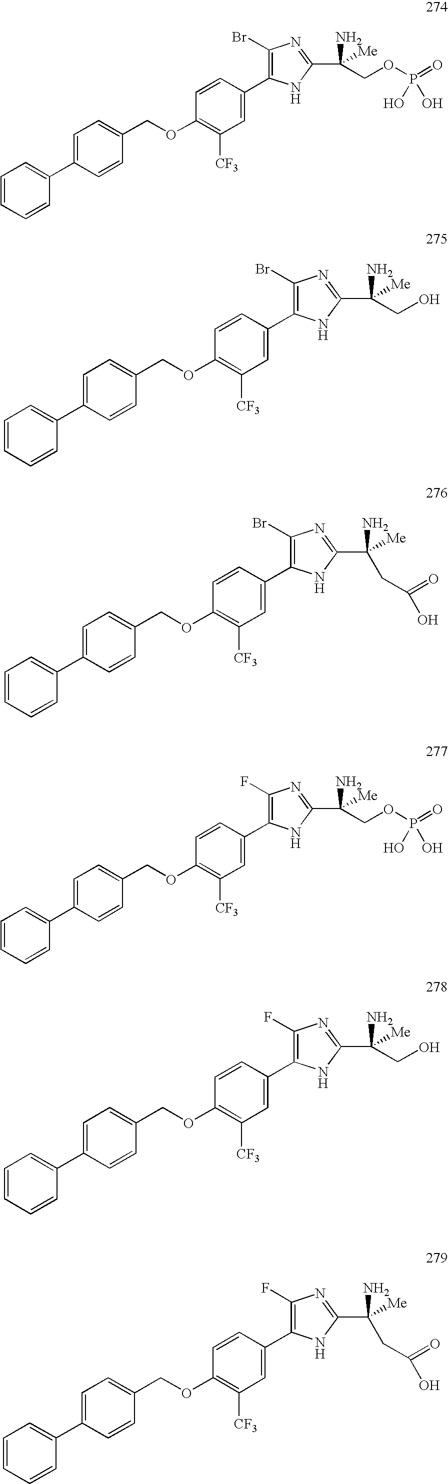

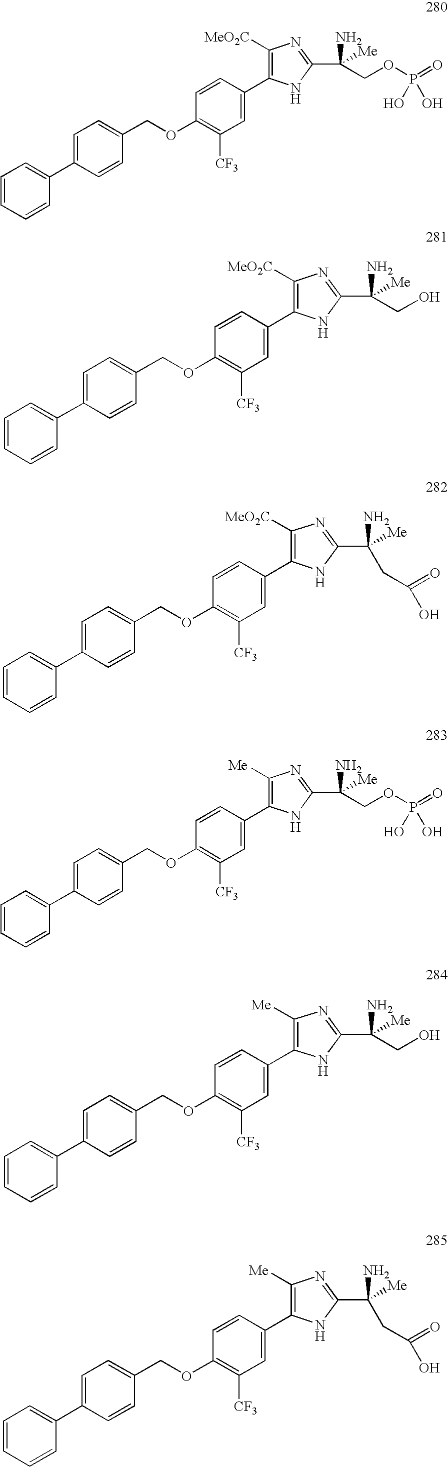

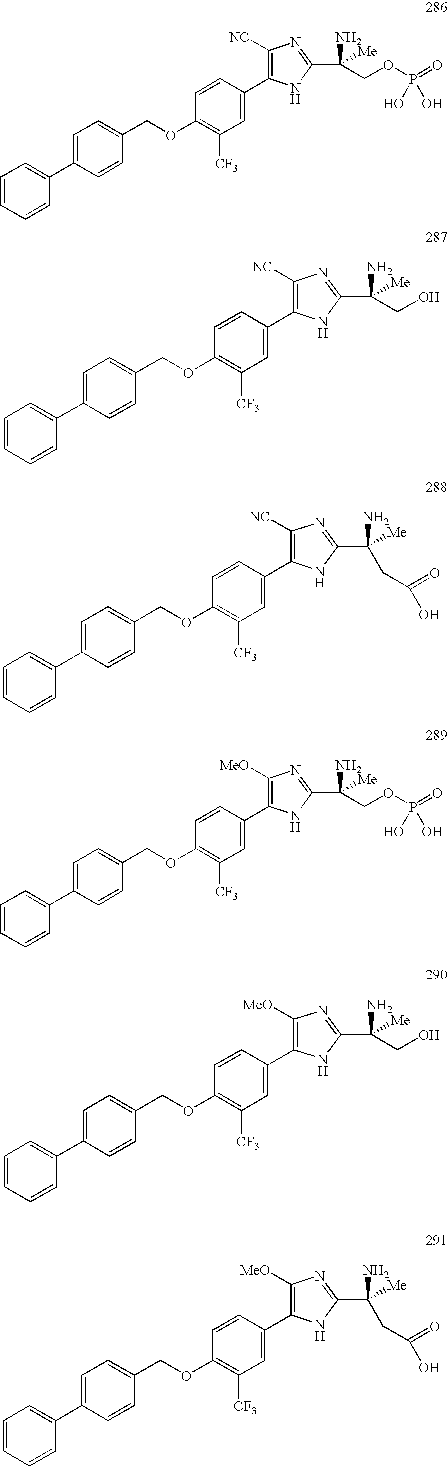

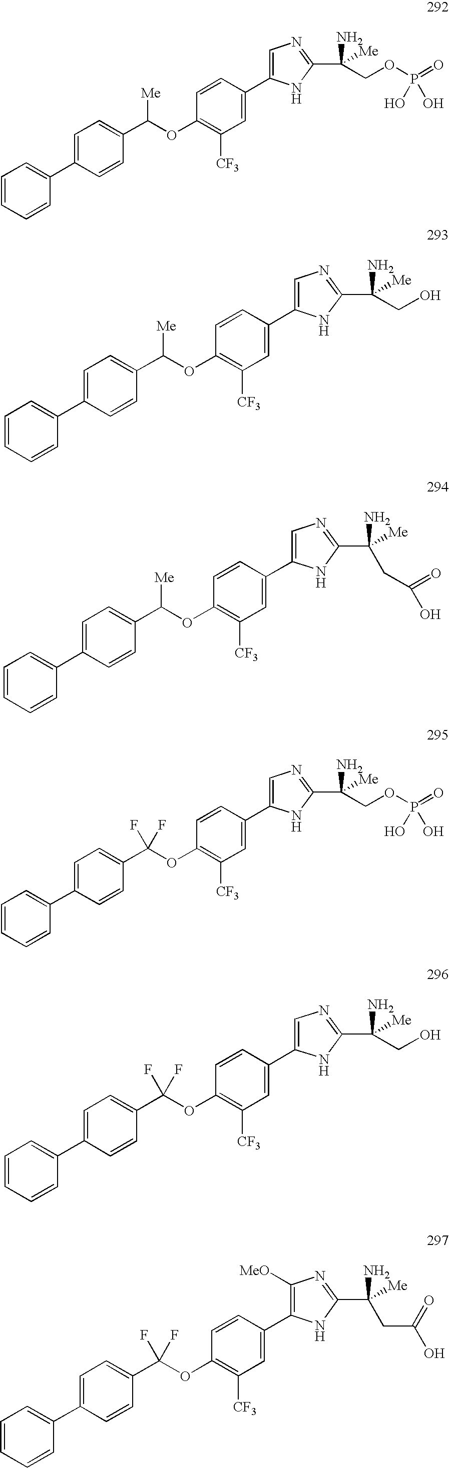

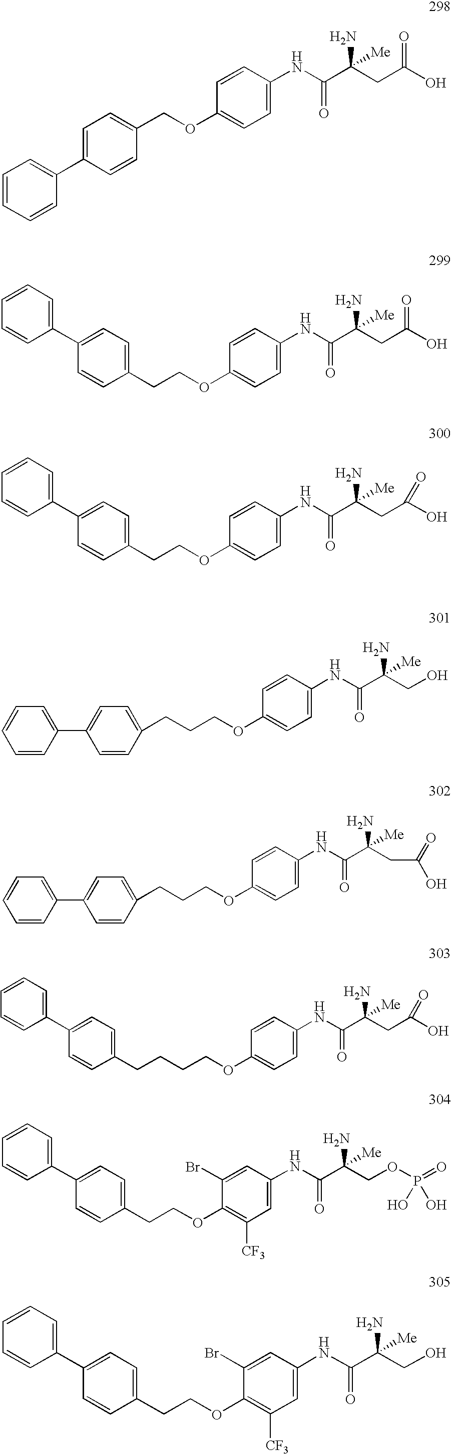

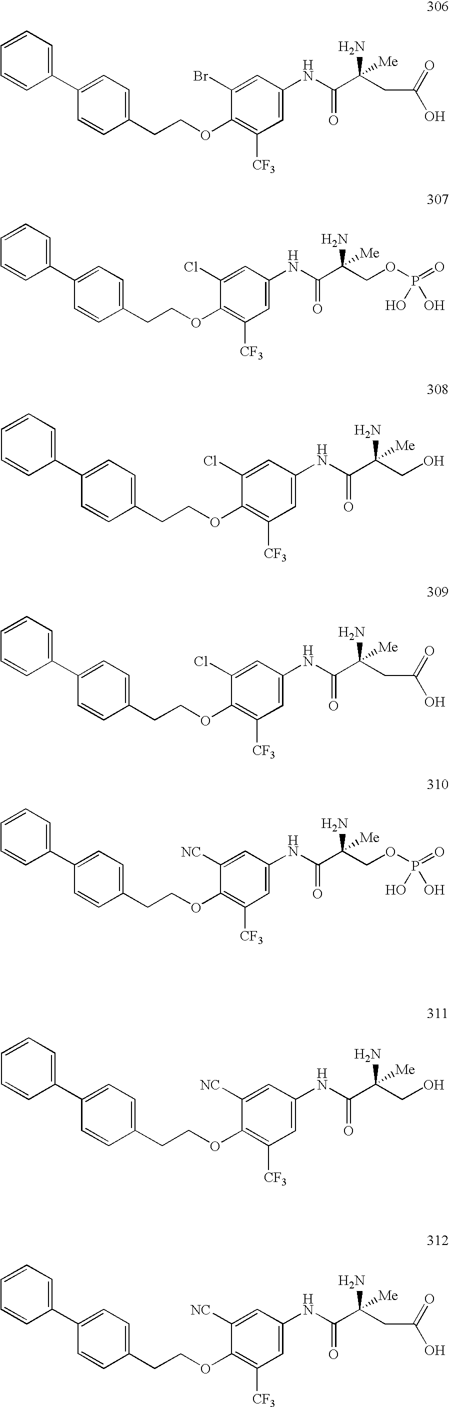

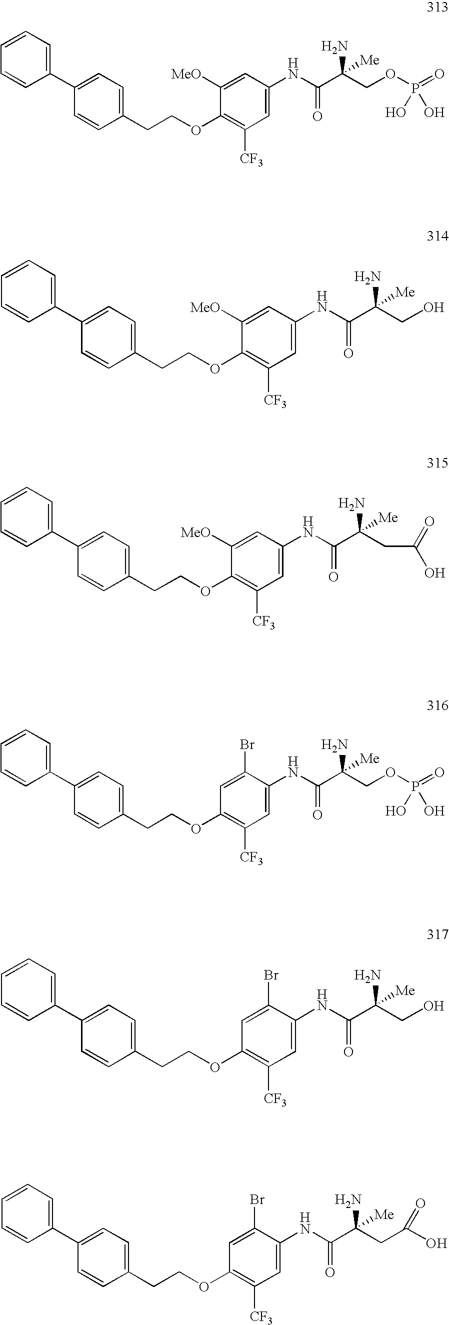

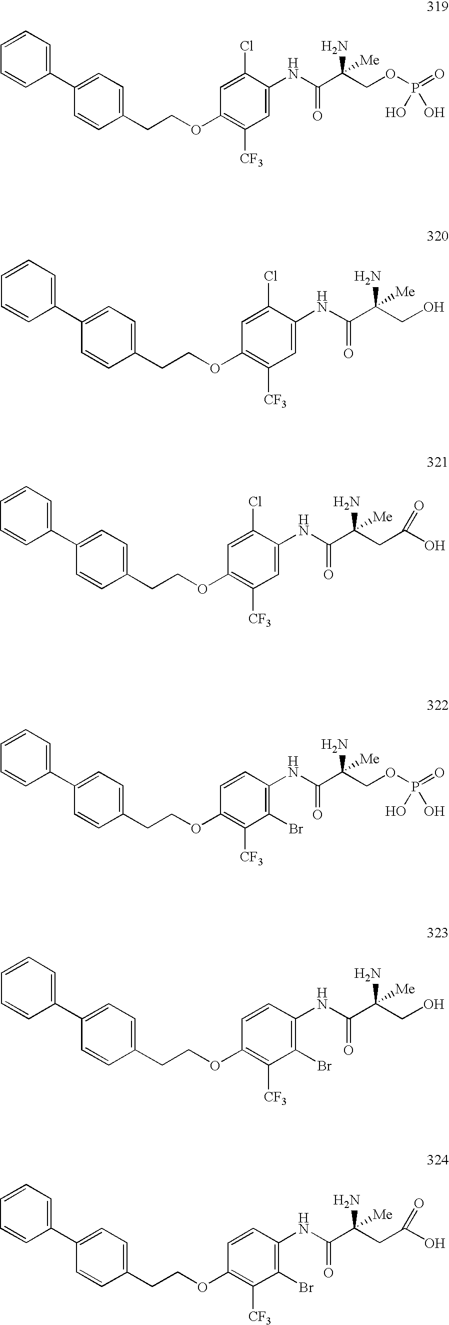

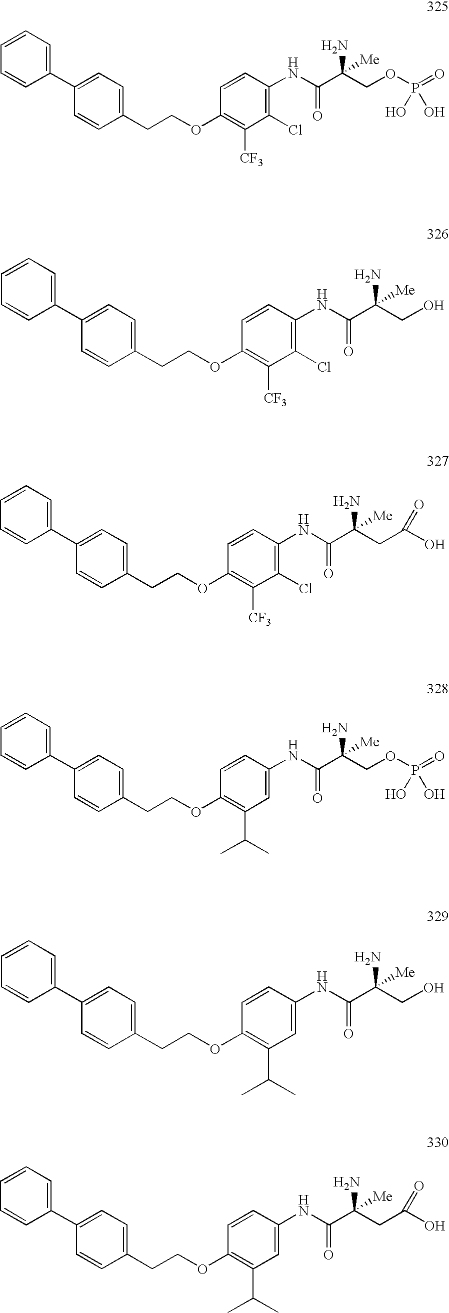

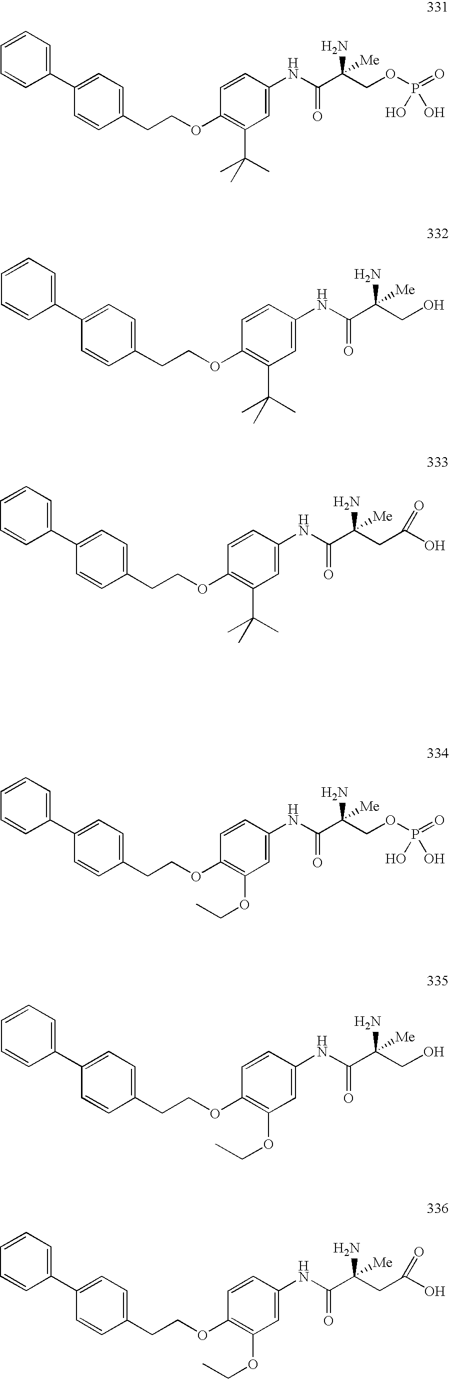

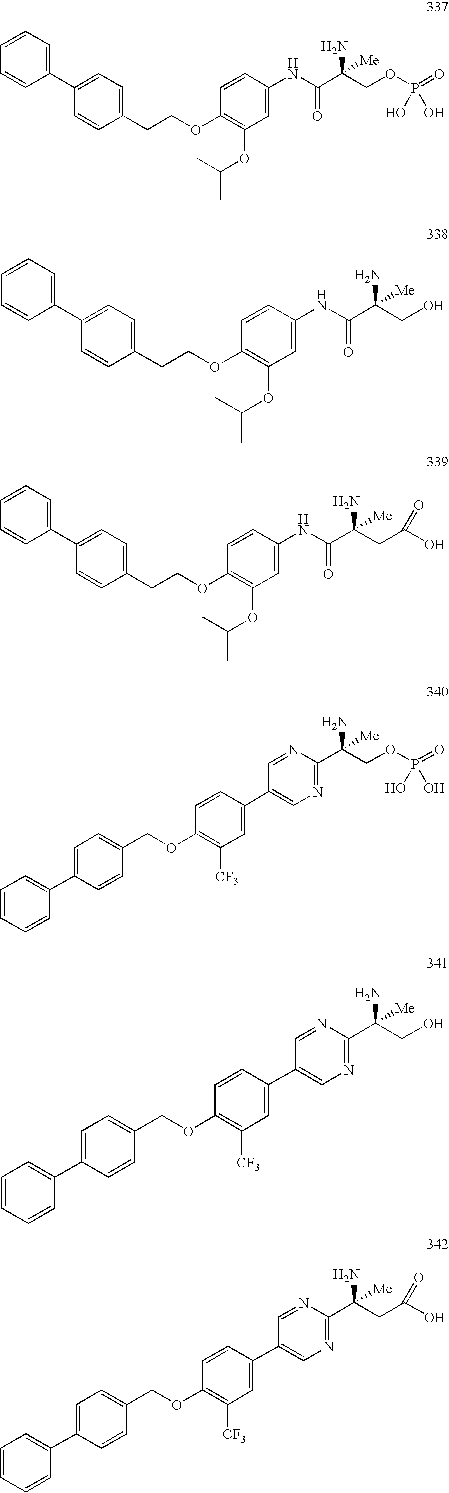

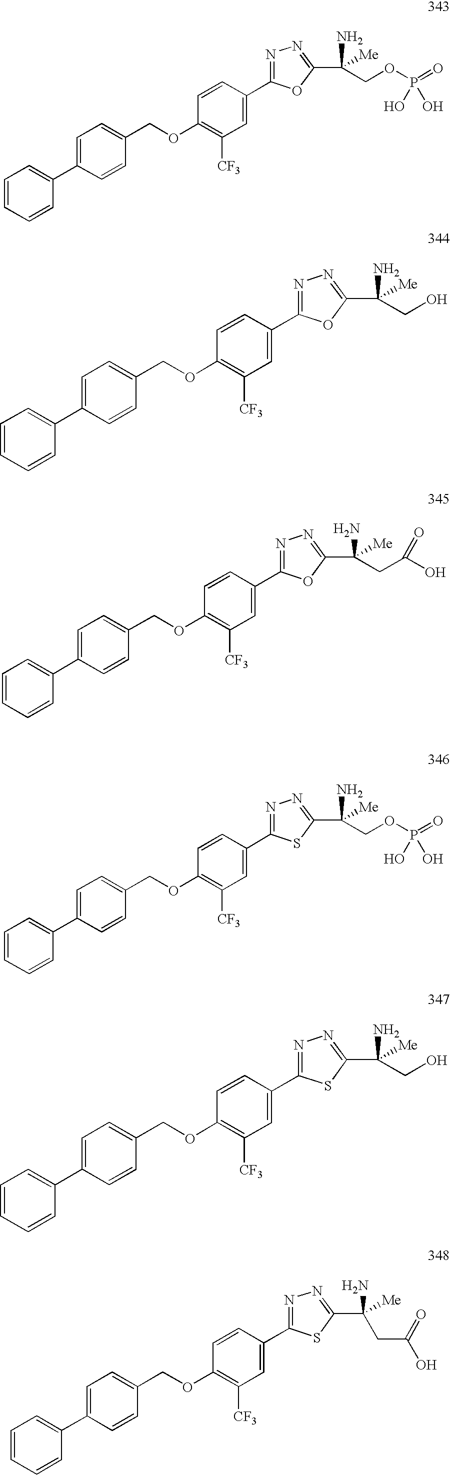

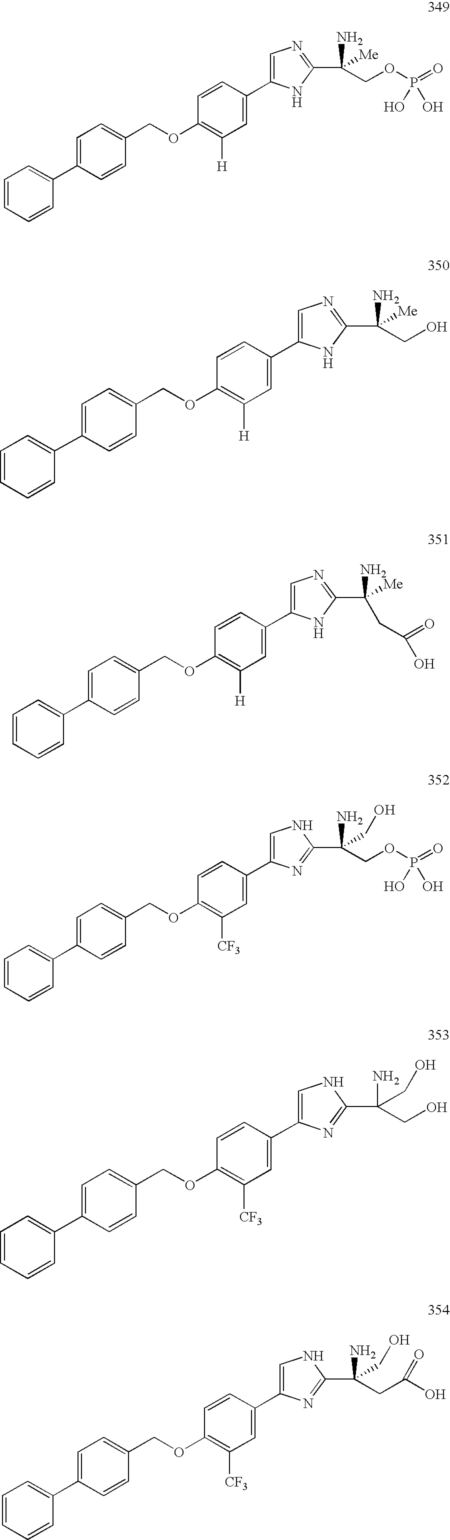

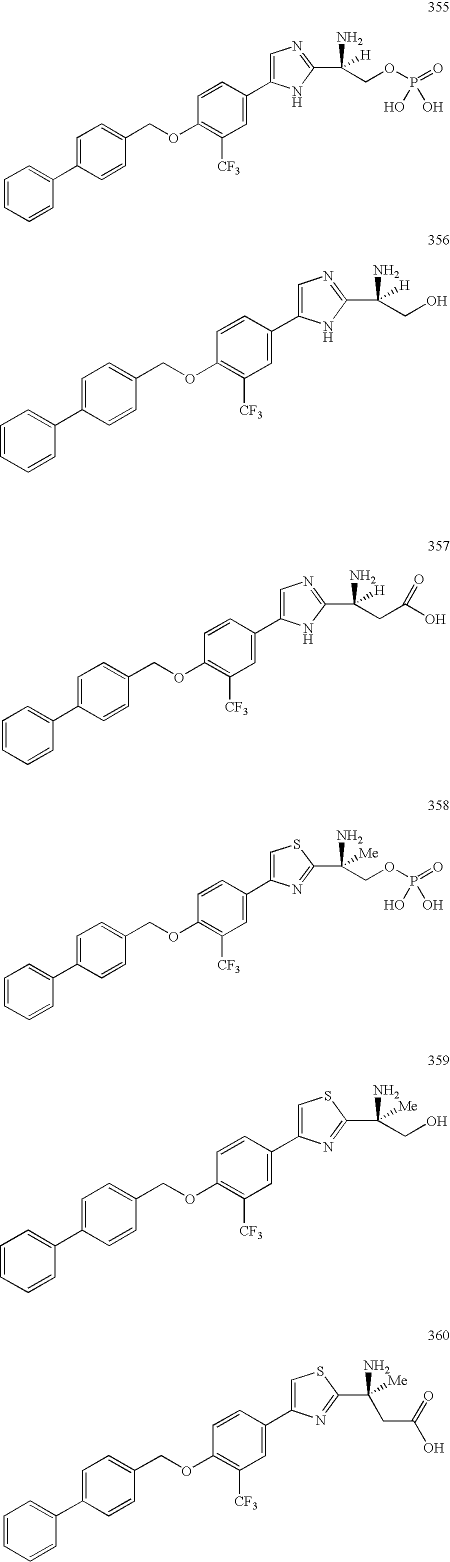

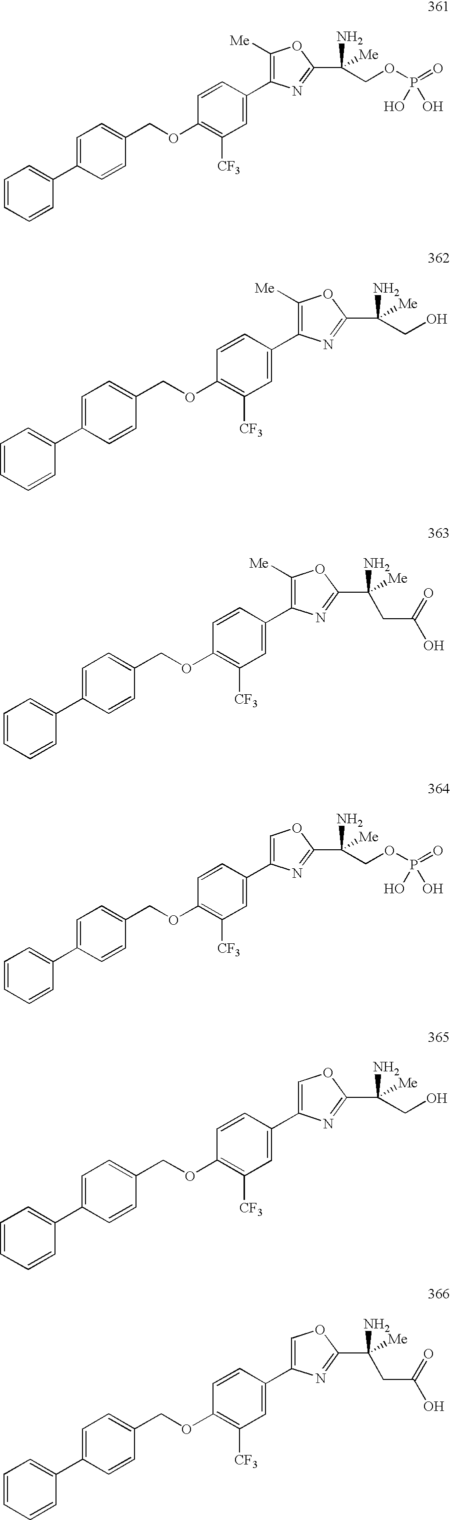

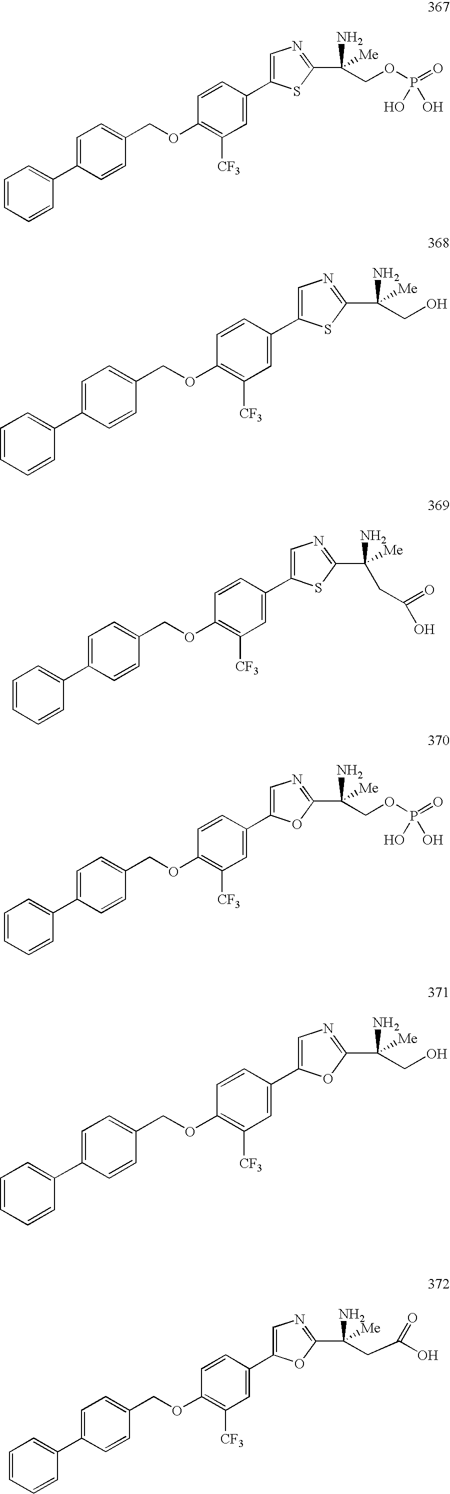

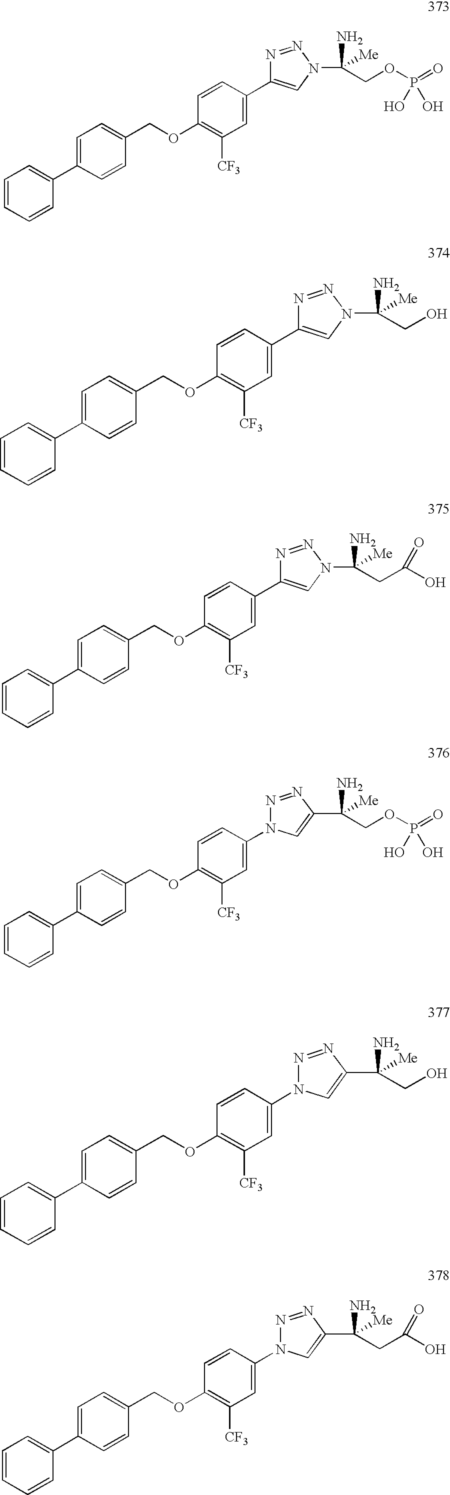

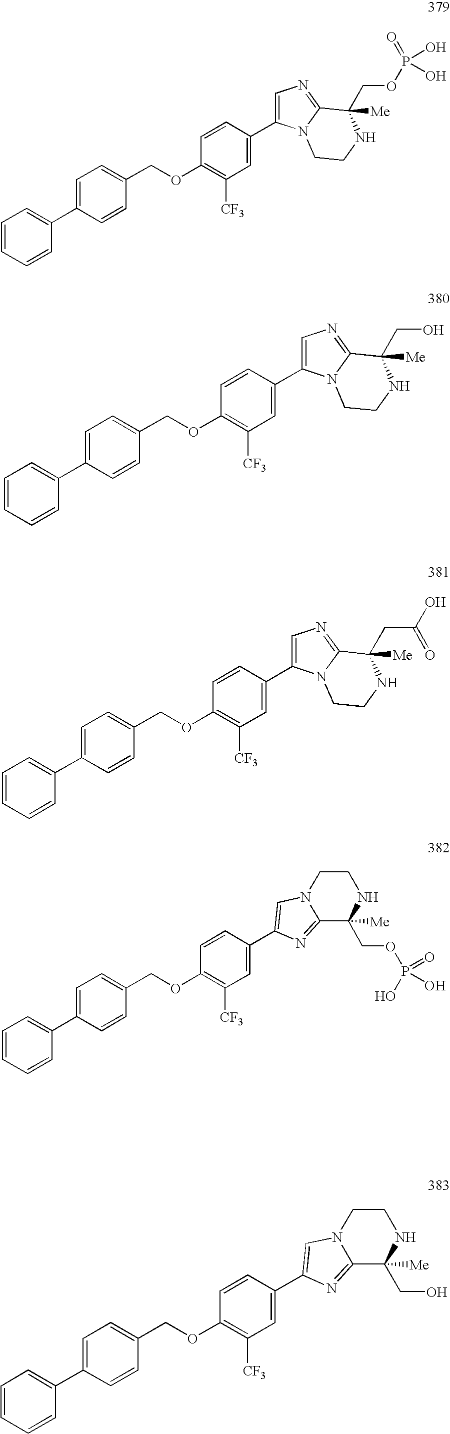

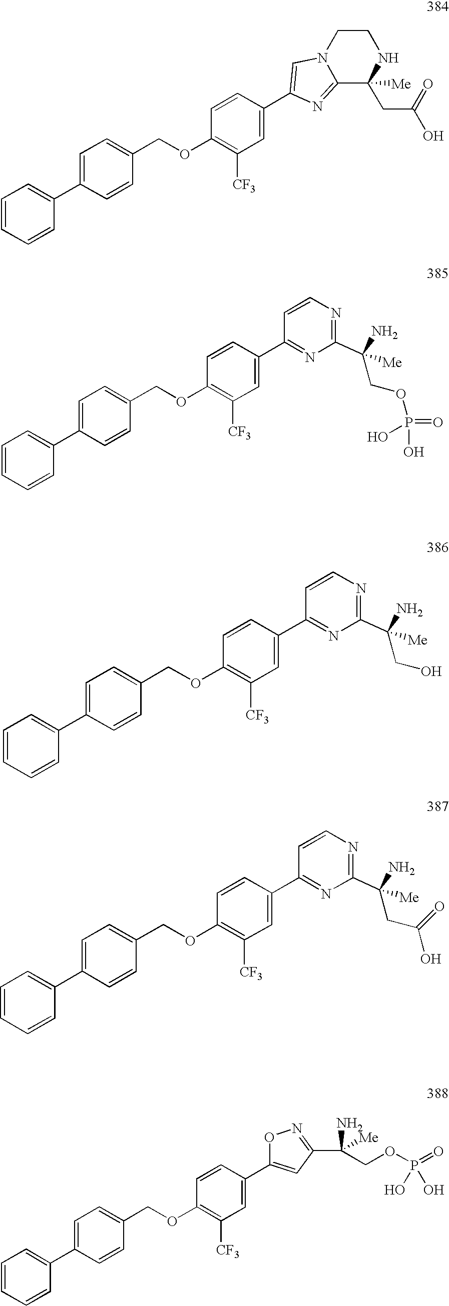

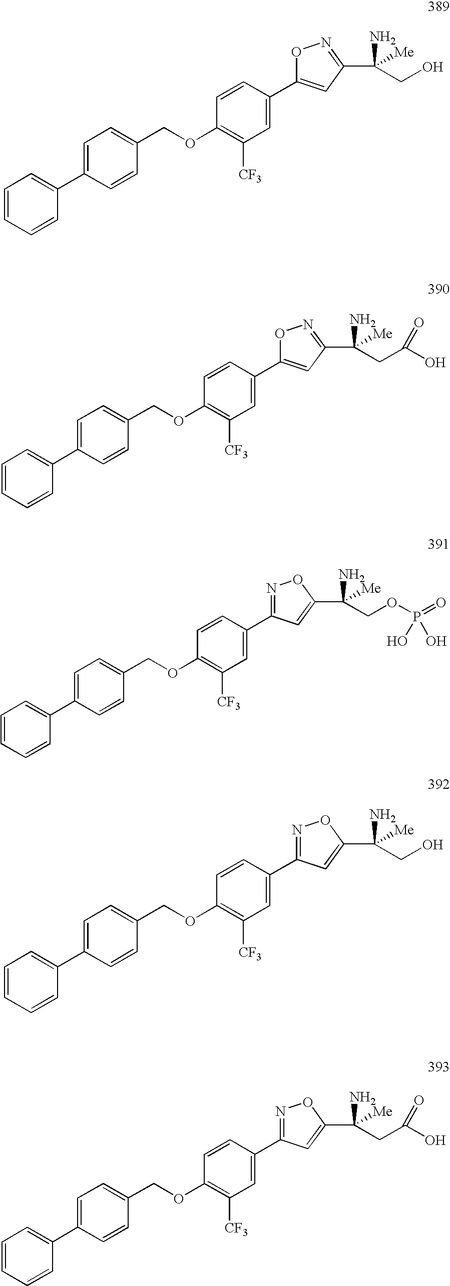

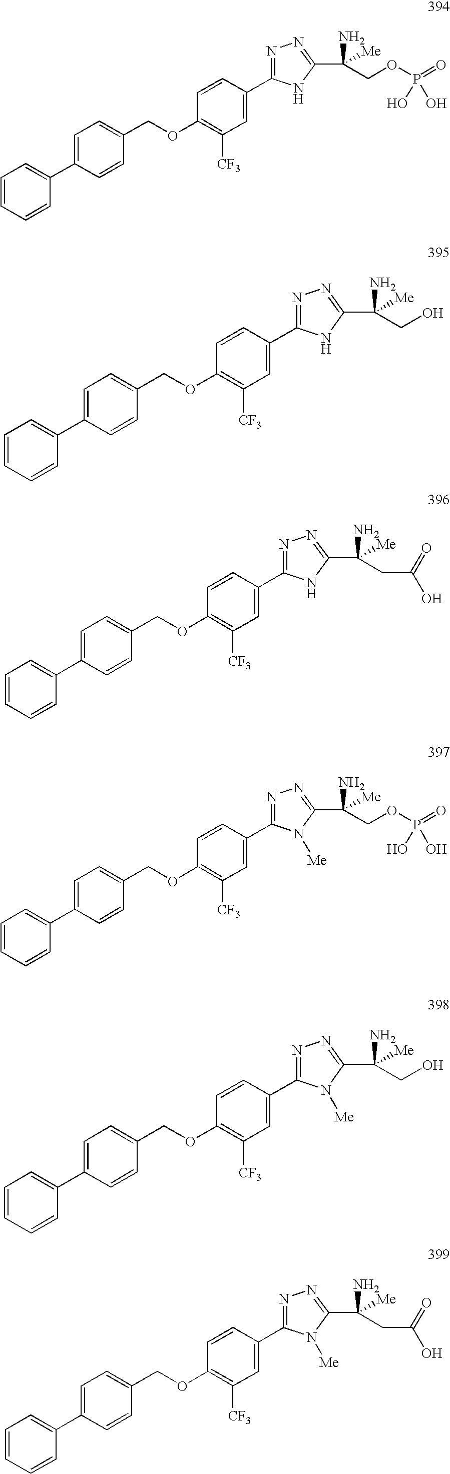

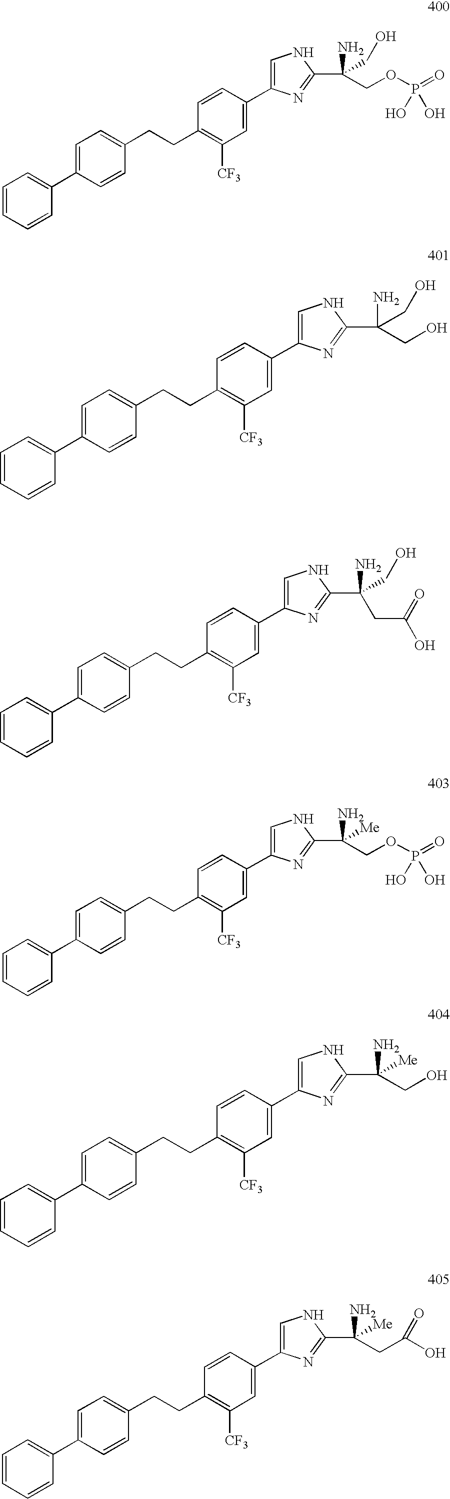

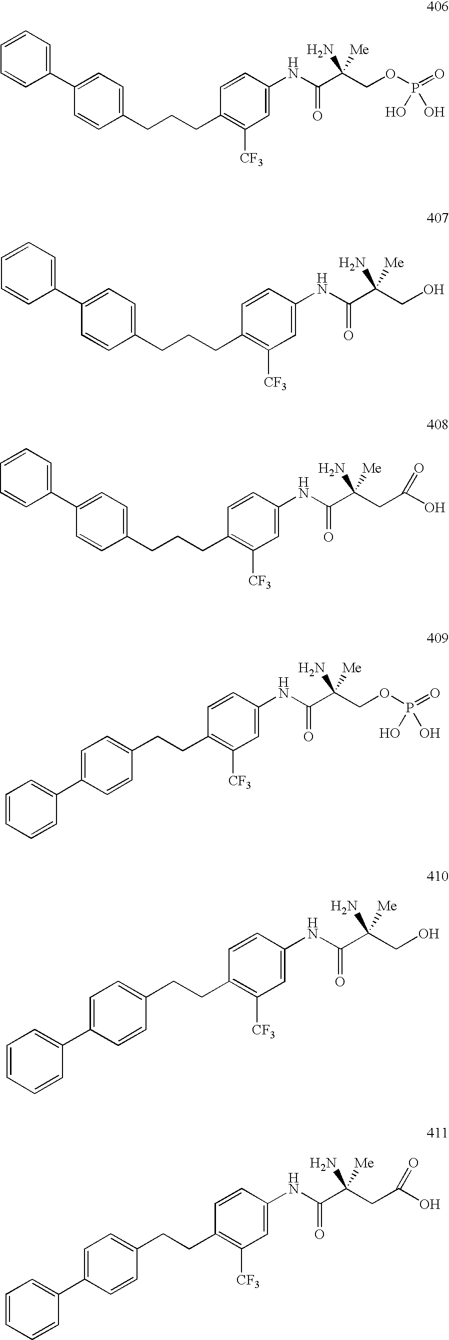

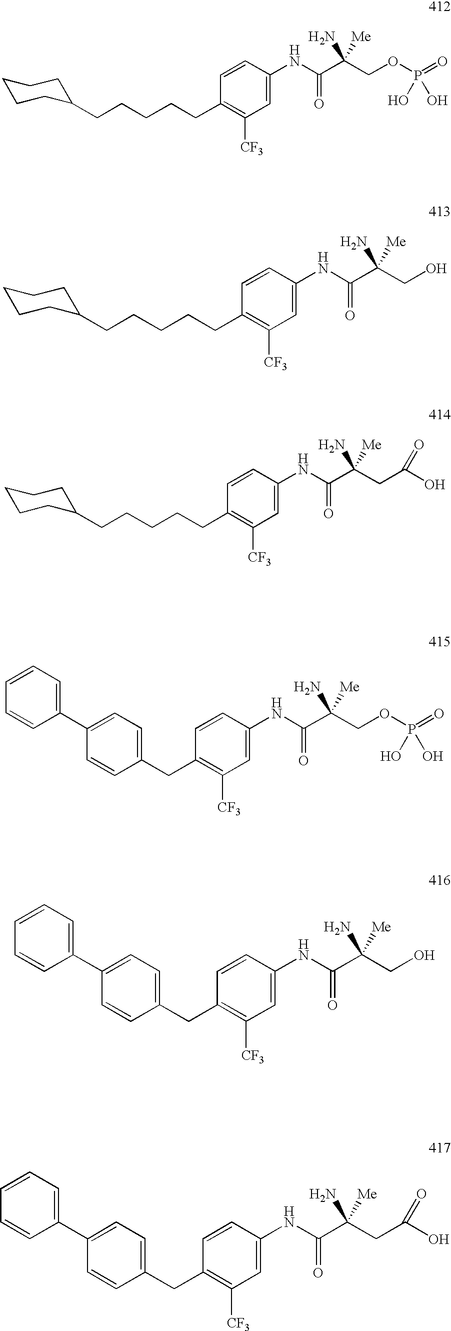

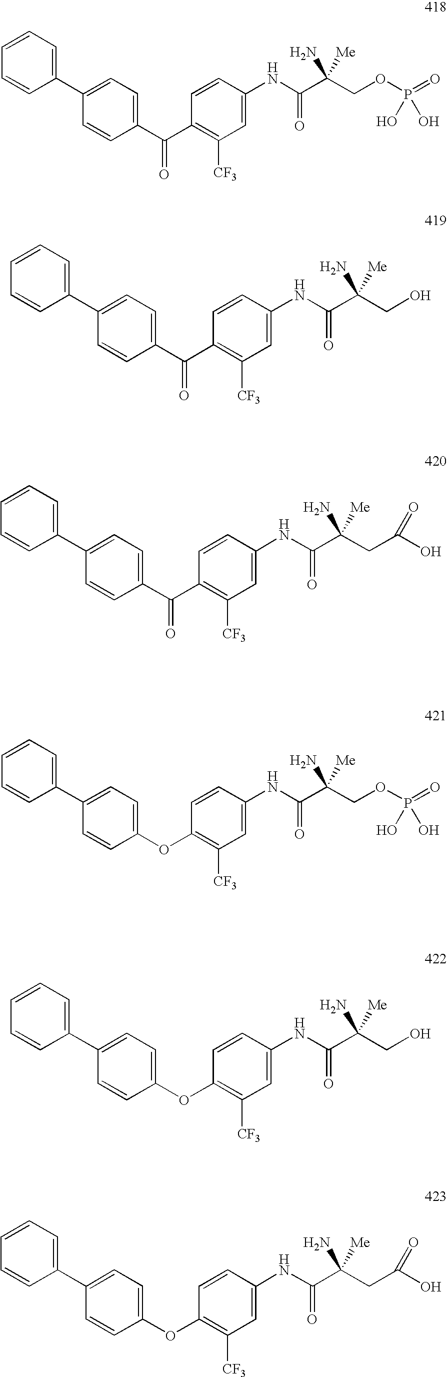

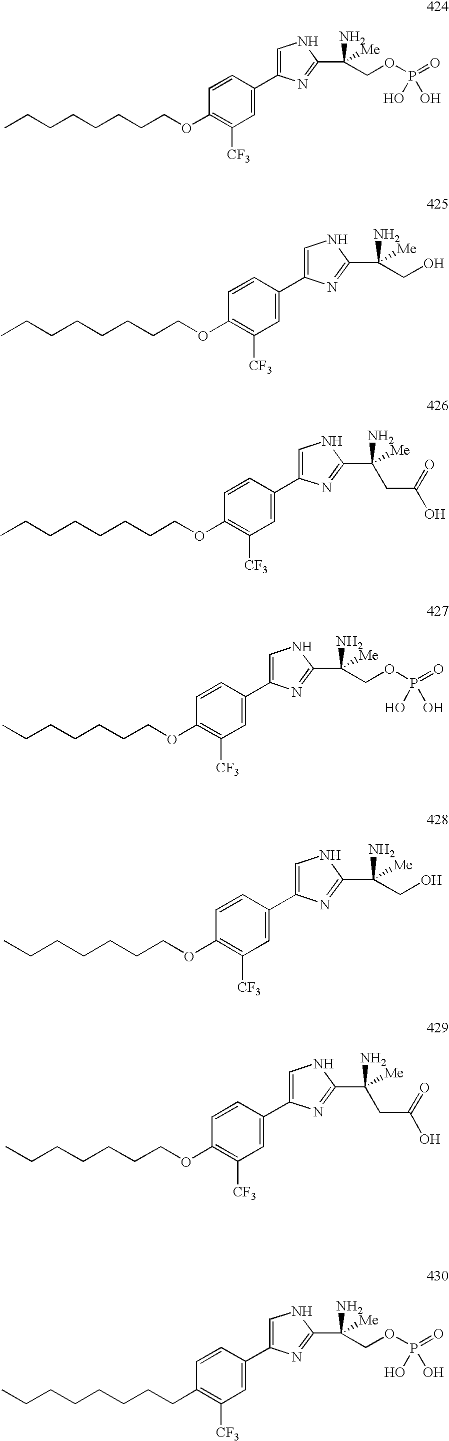

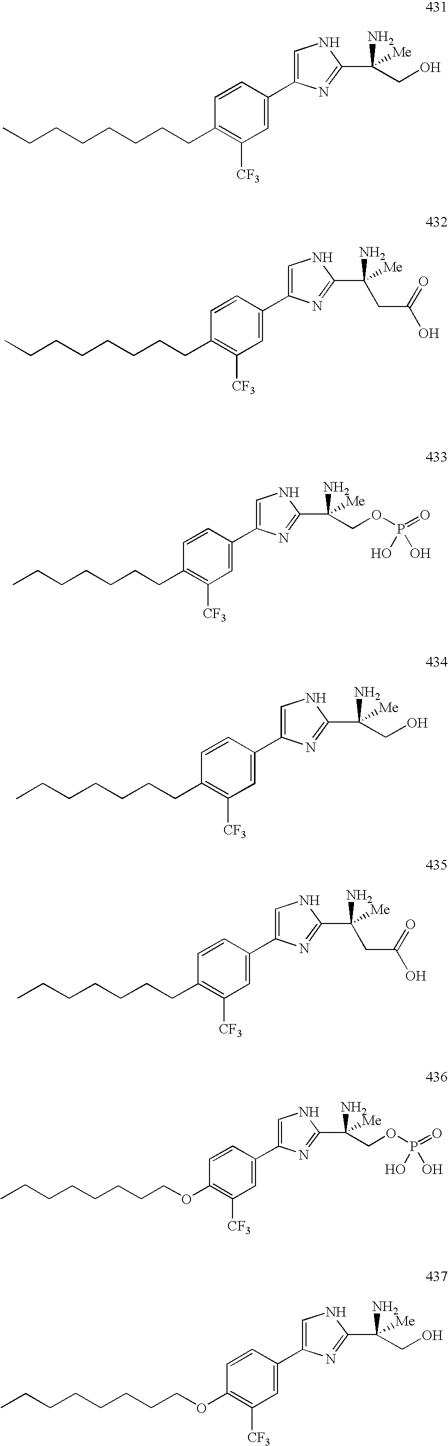

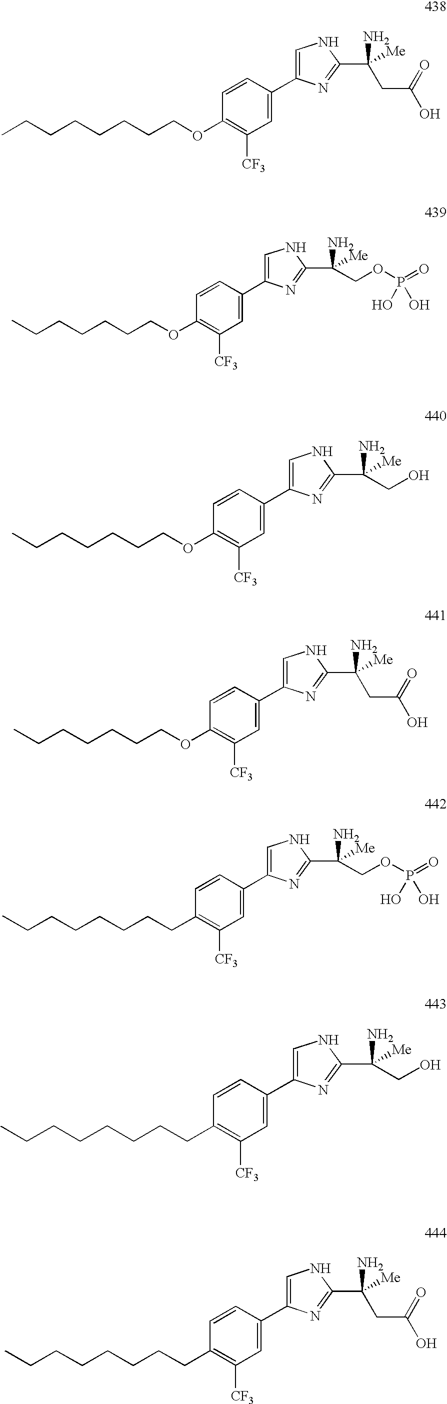

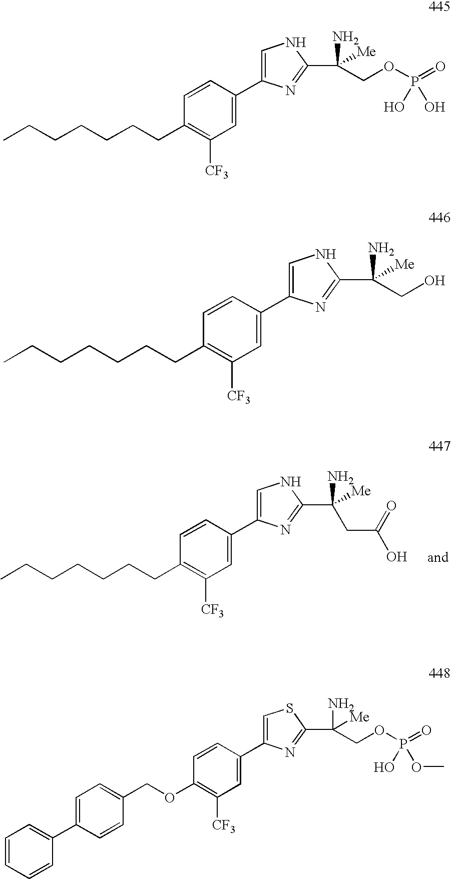

The invention also relates to salts of the compounds of the invention and, in particular, to pharmaceutically acceptable salts. A “pharmaceutically acceptable salt” includes a salt that retains the desired biological activity of the parent compound and does not impart any undesired toxicological effects. The salts can be, for example, salts with a suitable acid, such as hydrochloric acid, hydrobromic acid, sulfuric acid, phosphoric acid, nitric acid, and the like; acetic acid, oxalic acid, tartaric acid, succinic acid, malic acid, benzoic acid, pamoic acid, alginic acid, methanesulfonic acid, naphthalenesulfonic acid, and the like. Also included are salts of cations such as ammonium, sodium, potassium, lithium, zinc, copper, barium, bismuth, calcium, and the like; or organic cations such as tetralkylammonium and trialkylammonium cations. Combinations of the above salts are also useful. Salts of other acids and/or cations are also included, such as salts with trifluoroacetic acid, chloroacetic acid, and trichloroacetic acid.

The invention also includes different crystal forms, hydrates and solvates of the compounds of the invention, as well as stereoisomers of the compounds of the invention. Included are substantially pure single stereoisomers and mixtures of stereoisomers.

In a further embodiment, the compounds of any of Formulae I-XLVII is an agonist of a sphingosine 1-phosphate 1 receptor.

In certain embodiments, the compound of any of Formulae I-XLVII is an agonist of the S1P receptor.

In certain embodiments, the compound of any of Formulae I-XLVII is selective for the S1P1 receptor as compared to one or more of the other S1P receptors. For example, one set of compounds includes compounds which are selective for the S1P1 receptor relative to the S1P3 receptor. Compounds selective for the S1P1 receptor can be agonists of the S1P1 receptor, significantly weaker agonists of one or more other receptors and/or antagonists of one or more other receptors. A compound is “selective” for the S1P1 receptor relative to a second receptor, if the IC₅₀ of the compound for the second receptor is at least two-fold, e.g., at least 10-fold, e.g., at least 100-fold greater than the IC₅₀ for the S1P1 receptor. The IC₅₀ of a compound is determined using the ³⁵S-GTPγS binding assay, as described in WO 03/061567, the contents of which are incorporated herein by reference.

The terms “agonist” or “S1P1 receptor agonist” as used herein include the compounds described herein which bind to and/or agonize the S1P1 receptor. In one embodiment, the S1P receptor agonists have an IC₅₀ for the S1P1 receptor of about 100 nM-0.25 nM, about 50 nM-0.25 nM, about 25 nM-0.5 nM, about 100 nM or less, about 75 nM or less, about 50 nM or less, about 40 nM or less, about 30 nM or less, about 20 nM or less, about 10 nM or less, about 5 nM or less, about 1 nM or less, about 0.5 nM or less, or about 0.25 nM or less. The compounds' IC₅₀ for the S1P1 receptor can be measured using the binding assays described in Example 11 or those described in WO 03/061567.

Ranges intermediate to the above recited values are also intended to be part of this invention. For example, ranges using a combination of any of the above recited values as upper and/or lower limits are intended to be included.

In a further embodiment, the S1P receptor agonist has an IC₅₀ value for the S1P3 receptor of about 10 nM-10,000 nM, about 100 nM-5000 nM, about 100 nM-3000 nM, about 10 nM or greater, about 20 nM or greater, about 40 nM or greater, about 50 nM or greater, about 75 nM or greater, or about 100 nM or greater. In another embodiment, the S1P compound of the invention binds the S1P3 receptor with an IC₅₀ of 1000 nM or greater, 2000 nM or greater, 3000 nM or greater, 5000 nM or greater, 10,000 nM or greater. The IC₅₀ for of S1P3 receptor can be measured using the binding assays described in Example 11 or those described in WO 03/061567.

In addition, it should be understood that the ranges intermediate to the above recited values are also intended to be part of this invention. For example, ranges using a combination of any of the above recited values as upper and/or lower limits are intended to be included.

In yet another embodiment, the S1P receptor agonists described herein have an IC₅₀ value for the S1P1 receptor that is about 5-fold lower, about 10-fold lower, about 20-fold lower, about 50-fold lower, about 100-fold lower, about 200-fold lower, about 500-fold lower or about 1000-fold lower than their IC₅₀ value for the S1P3 receptor.

Again, ranges intermediate to the above recited values are also intended to be part of this invention. For example, ranges using a combination of any of the above recited values as upper and/or lower limits are intended to be included.

In a further embodiment, when Q is NH(C═O), O, or heteroaryl; R⁶ is OH; n is 1-4; one of R¹, R², R³, R⁴, and R⁵ is C₁-C₁₈ alkyl C₂-C₁₈ alkenyl, C₂-C₁₈ alkynyl, C₅-C₁₈-alkoxy, (CH₂)₁₋₁₀O(CH₂)₁₋₁₀, C₅-C₁₀(aryl), C₅-C₁₀(aryl)(C₁-C₁₀alkyl), C₅-C₁₀(heteroaryl), C₅-C₁₀(heteroaryl)(C₁-C₁₀alkyl), C₅-C₁₀ cycloalkyl, C₅-C₁₀(cycloalkyl)-(C₁-C₅ alkyl), C₅-C₁₀alkoxy(aryl), C₅-C₁₀alkoxy(aryl)(C₁-C₁₀ alkyl), C₅-C₁₀alkoxy(heteroaryl), C₅-C₁₀alkoxy(heteroaryl)(C₁-C₁₀ alkyl), C₅-C₁₀alkoxy(cycloalkyl), or C₅-C₁₀alkoxy(cycloalkyl)(C₁-C₁₀ alkyl); and one of R¹, R², R³, R⁴, and R⁵is H, halogen, NH₂, C₁-C₆ alkyl, C₁-C₆ alkoxy, C₁-C₆ alkylamino, C₁-C₆ alkylcyano, or C₁-C₆ alkylthio, R⁸ is not hydrogen.

In another further embodiment, when Q is heteroaryl; one of R¹, R², R³, R⁴, and R⁵ is alkyl, alkenyl, alkynyl, optionally substituted aryl, optionally substituted heteroaryl, alkyl (optionally substituted aryl), arylalkyl, or arylalkyl (optionally substituted (aryl); R⁸ is hydrogen; n is 1; R⁶ is not OH.

In another further embodiment, when Q is NH(C═O); R⁶ is OH; R¹, R², R³, R⁴, and R⁵ are each independently halogen, hydrogen, amino, or alkyl; R⁸ is not hydrogen.

In one embodiment, the compounds of the invention do not include the compounds described in WO 05/041899A2, WO 04/010949A2, WO 04/024673 A1 and WO 02/064616; the entire contents of each of which are hereby incorporated herein by reference.

In certain embodiments, the compounds of the present invention are characterized by a unique structure which imparts surprisingly improved properties to these compounds as compared to the prior art compounds. Specifically, the compounds of the present invention are characterized by the presence of a substituted biphenyl moiety. This biphenyl moiety, in combination with an amide linkage or heteroaryl moiety, e.g., imidazolyl, within the core of the structure, enhances the selectivity of the compounds described herein for the S1P 1 receptor versus other receptors, such as S1P3. In fact, many of the compounds of the present invention are further characterized by their potent binding to the S1P 1 receptor.

Methods of Using the Compounds of the Invention

The compounds of the invention have been determined to useful at least in the treatment of sphingosine 1-phosphate associated disorders. Accordingly, in one embodiment, the invention relates to a method for treating a subject suffering from a sphingosine 1-phosphate associated disorder, comprising administering to a subject an effective amount of a compound of the invention, e.g., compounds of any of Formulae I-XLVII or compounds otherwise described herein, such that the subject is treated for a sphingosine 1-phosphate associated disorder.

The term “sphingosine 1-phosphate associated disorder” includes disorders, diseases or conditions which are associated with or caused by a misregulation in S1P receptor function and/or signaling or S1P receptor ligand function. The term also includes diseases, disorders or conditions which can be treated by administering to a subject an effective amount of a sphingosine 1-phosphate receptor agonist. Such disorders include disorders that are associated with an inappropriate immune response and conditions associated with an overactive immune response.

An additional embodiment of the invention pertains to a method for treating a subject suffering from a sphingosine 1-phosphate associated disorder, comprising administering to a subject a compound, such that the subject is treated for a sphingosine 1-phosphate associated disorder by a compound of the invention, e.g., compounds of any of Formulae I-XLVII or compounds otherwise described herein.

In an additional embodiment, the present invention is directed to a method of selectively treating a sphingosine 1-phosphate associated disorder, comprising administering to a subject an effective amount of a compound of the invention, e.g., compounds of any of Formulae I-XLVII or compounds otherwise described herein, such that the subject is selectively treated for a sphingosine 1-phosphate associated disorder. In certain embodiments, the sphingosine 1-phosphate associated disorder is a sphingosine 1-phosphate-(1) associated disorder. In a particular embodiment, the sphingosine 1-phosphate-(1) associated disorder is selectively treated as compared with a sphingosine 1-phosphate-(3) associated disorder.

Another embodiment of the invention is a method of selectively treating a sphingosine 1-phosphate associated disorder, comprising administering to a subject a compound, such that the subject is selectively treated for a sphingosine 1-phosphate associated disorder by a compound of the invention, e.g., compounds of any of Formulae I-XLVII or compounds otherwise described herein. In certain embodiments, the sphingosine 1-phosphate associated disorder is a sphingosine 1-phosphate-(1) associated disorder. In a particular embodiment, the sphingosine 1-phosphate-(1) associated disorder is selectively treated as compared with a sphingosine 1-phosphate-(3) associated disorder.

In another embodiment, the present invention provides a method of treating a condition associated with an overactive immune response. An “overactive immune response” is an undesirable or inappropriate immune response and in conditions associated with an overactive immune response, the immune response is deleterious to the subject. Included are conditions such as autoimmune disorders, organ and tissue transplants, including transplant rejection and graft versus host disease, diabetes and chronic inflammatory disorders. The method includes administering to the subject a therapeutically effective amount of a compound of the present invention, thereby treating the condition associated with an overactive immune response in the subject.

The compounds of the invention can be used to treat subjects undergoing, or who have undergone, an organ, tissue or cell transplant from a donor. In one embodiment, the transplanted tissue, organ or cell is bone marrow, stem cells, pancreatic cells, such as islet cells, or cornea. In another embodiment, the transplanted organ is a solid organ, such as a liver, a kidney, a heart or a lung.

Autoimmune disorders which can be treated with the compounds of the invention include systemic lupus erythematosus, rheumatoid arthritis, multiple sclerosis, myasthenia gravis, type 1 diabetes, ankylosing spondylitis, psoriatic arthritis, scleroderma, Kawasaki syndrome and other rheumatic diseases as set forth in Primer on the Rheumatic Diseases, 11th Edition (John H. Klippel MD, editor; Arthritis Foundation:Atlanta Ga. (1997)).

Other autoimmune diseases that can be treated with the present compounds include active chronic hepatitis, Addison's Disease, anti-phospholipid syndrome, atopic allergy, autoimmune atrophic gastritis, achlorhydra autoimmune, Celiac Disease, Crohn's Disease, Cushing's Syndrome, dermatomyositis, Goodpasture's Syndrome, Grave's Disease, Hashimoto's thyroiditis, idiopathic adrenal atrophy, idiopathic thrombocytopenia, Lambert-Eaton Syndrome, lupoid hepatitis, mixed connective tissue disease, pemphigoid, pemphigus vulgaris, pernicious anemia, phacogenic uveitis, polyarteritis nodosa, primary biliary cirrhosis, primary sclerosing cholangitis, psoriasis, Raynauds, Reiter's Syndrome, relapsing polychondritis, Schmidt's Syndrome, Sjogren's Syndrome, sympathetic ophthalmia, Takayasu's Arteritis, temporal arteritis, thyrotoxicosis, Type B Insulin Resistance, ulcerative colitis, and Wegener's granulomatosis.

As used herein, the term “subject” includes warm-blooded animals, e.g., mammals, including humans, cats, dogs, horses, bears, lions, tigers, ferrets, rabbits, mice, cows, sheep, pigs, etc. In a particular embodiment, the subject is a primate. In a specific embodiment, the primate is a human.

As used herein, the term “administering” to a subject includes dispensing, delivering or applying a compound of the invention in a pharmaceutical formulation (as described herein), to a subject by any suitable route for delivery of the compound to the desired location in the subject, including delivery by either the parenteral or oral route, intramuscular injection, subcutaneous/intradermal injection, intravenous injection, buccal administration, topical delivery, transdermal delivery and administration by the rectal, colonic, vaginal, intranasal or respiratory tract route.

As used herein, the term “effective amount” includes an amount effective, at dosages and for periods of time necessary, to achieve the desired result, e.g., sufficient to treat the condition in a subject. An effective amount of a compound of the invention, as defined herein, may vary according to factors such as the disease state, age, and weight of the subject, and the ability of the compound to elicit a desired response in the subject. Dosage regimens may be adjusted to provide the optimum therapeutic response. An effective amount is also one in which any toxic or detrimental effects (e.g., side effects) of the compound are outweighed by the therapeutically beneficial effects.

A therapeutically effective amount of a compound of the invention (i.e., an effective dosage) may range from about 0.001 to 30 mg/kg body weight, for example, about 0.01 to 25 mg/kg body weight, for example, about 0.1 to 20 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a compound of the invention can include a single treatment or, for example, can include a series of treatments. It will also be appreciated that the effective dosage of the compound used for treatment may increase or decrease over the course of a particular treatment.

The methods of the invention further include administering to a subject a therapeutically effective amount of a compound of the invention in combination with another pharmaceutically active compound known to treat the disease or condition, e.g., an immunomodulatory agent or an anti-inflammatory agent. Pharmaceutically active compounds that may be used depend upon the condition to be treated, but include as examples cyclosporin, rapamycin, FK506, methotrexate, etanercept, infliximab, adalimumab, non-steroidal anti-inflammatory agents, cyclooxygenase-2-inhibitors, such as celecoxib and rofecoxib, and corticosteroids. Other suitable compounds can be found in Harrison's Principles of Internal Medicine, Thirteenth Edition, Eds. T. R. Harrison et al. McGraw-Hill N.Y., N.Y.; and the Physicians Desk Reference 50th Edition 1997, Oradell N.J., Medical Economics Co., the complete contents of which are expressly incorporated herein by reference. The compound of the invention and the additional pharmaceutically active compound may be administered to the subject in the same pharmaceutical composition or in different pharmaceutical compositions (at the same time or at different times).

Pharmaceutical Compositions of the Compounds of the Invention

The present invention also provides pharmaceutically acceptable formulations and compositions comprising one or more compounds of the invention, e.g., compounds of any of Formulae I-XLVII or compounds otherwise described herein. In certain embodiments, the compound of the invention is present in the formulation in a therapeutically effective amount, e.g., an amount effective to treat a sphingosine 1-phosphate associated disorder.

Accordingly, in one embodiment, the invention pertains to a pharmaceutical composition comprising a therapeutically effective amount of a compound of the invention, e.g., compounds of any of Formulae I-XLVII or compounds otherwise described herein, and a pharmaceutically acceptable carrier.

In another embodiment, the invention is directed to a packaged pharmaceutical composition comprising a container holding a therapeutically effective amount of a compound of the invention, e.g., compounds of any of Formulae I-XLVII or compounds otherwise described herein; and instructions for using the compound to treat a sphingosine 1-phosphate associated disorder in a subject.

The term “container” includes any receptacle for holding the pharmaceutical composition. For example, in one embodiment, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions can contain information pertaining to the compound's ability to perform its intended function, e.g., treating, preventing, or reducing a sphingosine 1-phosphate associated disorder in a subject.

Another embodiment of the invention relates to a packaged pharmaceutical composition comprising a container holding a therapeutically effective amount of a compound of the invention, e.g., compounds of any of Formulae I-XLVII or compounds otherwise described herein, and instructions for using the compound to selectively treat a sphingosine 1-phosphate associated disorder in a subject.

Such pharmaceutically acceptable formulations typically include one or more compounds of the invention as well as one or more pharmaceutically acceptable carriers and/or excipients. As used herein, “pharmaceutically acceptable carrier” includes any and all solvents, dispersion media, coatings, antibacterial and antifungal agents, isotonic and absorption delaying agents, and the like that are physiologically compatible. The use of such media and agents for pharmaceutically active substances is well known in the art. Except insofar as any conventional media or agent is incompatible with the compounds of the invention, use thereof in the pharmaceutical compositions is contemplated.

Supplementary pharmaceutically active compounds known to treat transplant or autoimmune disease, i.e., immunomodulatory agents and anti-inflammatory agents, as described above, can also be incorporated into the compositions of the invention. Suitable pharmaceutically active compounds that may be used can be found in Harrison's Principles of Internal Medicine (supra).

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injection include sterile aqueous solutions (where water soluble) or dispersions, or sterile powders for the extemporaneous preparation of sterile injectable solutions or dispersions. For intravenous administration, suitable carriers include physiological saline, bacteriostatic water, Cremophor EI™ (BASF, Parsippany, N.J.) or phosphate buffered saline (PBS). In all cases, the pharmaceutical composition must be sterile and should be fluid to the extent that easy syringability exists. It must also be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, aluminum monostearate and gelatin.

Sterile injectable solutions can be prepared by incorporating the compound of the invention in the required amount in an appropriate solvent with one or a combination of the ingredients enumerated above, as needed, followed by filtered sterilization. Generally, dispersions are prepared by incorporating the compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the compound plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the compound of the invention can be incorporated with excipients and used in the form of tablets, troches, or capsules. Oral compositions can also include an enteric coating. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds of the invention are delivered in the form of an aerosol spray from a pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the compounds of the invention are formulated into ointments, salves, gels, or creams as generally known in the art.

The present pharmaceutical compositions can also be prepared in the form of suppositories (e.g., with conventional suppository bases such as cocoa butter and other glycerides) or retention enemas for rectal delivery.

In one embodiment, the compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions can also be used as pharmaceutically acceptable carriers. These can be prepared according to methods known to those skilled in the art, for example, as described in U.S. Pat. No. 4,522,811, U.S. Pat. No. 5,455,044 and U.S. Pat. No. 5,576,018, and U.S. Pat. No. 4,883,666, the contents of all of which are incorporated herein by reference.

The compounds of the invention can also be incorporated into pharmaceutical compositions which allow for the sustained delivery of the compounds to a subject for a period of at least several weeks to a month or more. Such formulations are described in published PCT application no. WO 02/74247, incorporated herein by reference.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of a compound of the invention calculated to produce the desired therapeutic effect in association with the required pharmaceutical carrier. The specification for the unit dosage forms of the invention are dictated by and directly dependent on the unique characteristics of the compound and the particular therapeutic effect to be achieved, and the limitations inherent in the art of compounding such compounds for the treatment of individuals.

This invention is further illustrated by the following examples, which should not be construed as limiting. The contents of all references, patents, patent applications cited throughout this application are incorporated herein by reference. It should be understood that the use of any of the compounds described herein are within the scope of the present invention and are intended to be encompassed by the present invention and are expressly incorporated herein for all purposes.

### EXAMPLES

### Example 1

### Synthesis of Phenylamide Compounds with Alkoxy Tail Group

Certain of the target compounds were synthesized using either the method illustrated in Scheme 1 or the method illustrated in Scheme 2. In Scheme 1, alkylation of the hydroxyl group of a substituted aminophenol is achieved using alkyl bromide and a catalytic amount of NaI in the presence of either Cs₂CO₃ in DMF (60° C.) or KO^{t}Bu in acetone (50° C.). The amino group of the desired intermediate is then acylated with Boc-protected amino acid using either N-ethylcarbodiimide (EDC), 1-hydroxybenzotriazole (HOBt), and N,N-diisopropylethylamine (DIPEA) in CH₂Cl₂ or O-(7-azabenzotriazol-1-yl)-1,1,3,3-tetramethyluronium hexafluorophosphate (HATU) and DIPEA in DMF. The final compound was obtained in good yields from Boc deprotection of the later intermediate with 30% trifluoroacetic acid (TFA) in CH₂Cl₂. Scheme 2 provides an alternative approach to synthesis of the desired final compound in which the amino group of the aminophenol is acylated first, followed by alkylation of the hydroxyl residue. 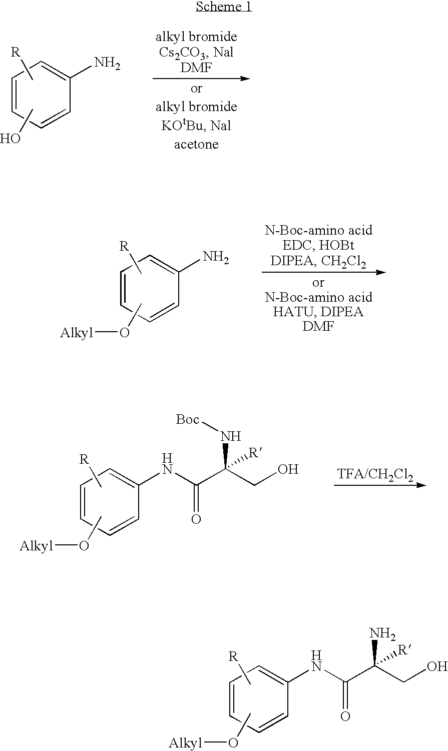 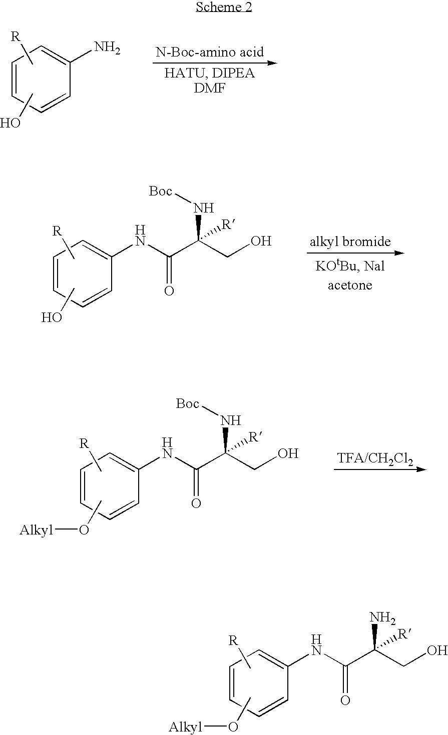
Alkylation of Hydroxyl Group

To a solution of desired substituted aminophenol (0.50 g, 1.0 equiv) and NaI (0.1 equiv) in acetone (10 mL) was added a 1.0 M solution of KO^{t}Bu in tetrahydrofuran (THF) (1.1 equiv, 2.1 equiv was used if aminophenol was a hydrochloride salt). To the reaction mixture was added the desired alkyl bromide (1.1 equiv). The reaction was stirred and heated under an atmosphere of nitrogen at 50° C. for 12-24 hours. The reaction was then diluted with EtOAc (25 mL) and washed with H₂O (2×25 mL) and saturated NaCl (1×25 mL). The organic layer was dried over anhydrous MgSO₄ then the solvent removed in vacuo. The crude produce was purified using silica gel column chromatography (3:1 Hex:EtOAc).

### 4-(Heptyloxy)benzenamine

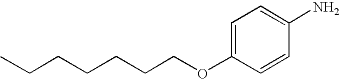

The product was obtained as a yellowish-brown solid in 71% (0.47 g) yield. TLC (3:1 Hex:EtOAc), R_{f}=0.4; ¹H NMR (400 MHz, CDCl₃) δ 6.69-6.74 (m, 2H), 6.59-6.63 (m, 2H), 3.86 (t, 2H, J=6.8 Hz), 3.40 (br s, 2H), 1.68-1.78 (m, 2H), 1.21-1.48 (m, 8H), 0.88 (t, 3H, J=6.8 Hz).

### 4-(Octyloxy)benzenamine

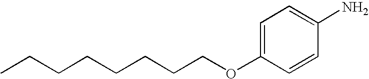

The product was obtained as brownish thick oil in 59% (0.45 g) yield. TLC (3:1 Hex:EtOAc), R_{f}=0.4; ¹H NMR (400 MHz, CDCl₃) δ 6.69-6.74 (m, 2H), 6.59-6.63 (m, 2H), 3.86 (t, 2H, J=6.9 Hz), 3.41 (br s, 2H), 1.69-1.79 (m, 2H), 1.22-1.47 (m, 10H), 0.88 (t, 3H, J=7.1 Hz).

### 3-Chloro-4-(heptyloxy)benzenamine

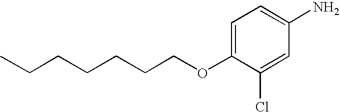

The product was obtained as a white solid in 51% (0.43 g) yield. TLC (3:1 Hex:EtOAc), R_{f}=0.5; ¹H NMR (400 MHz, CDCl₃) δ 6.74 (d, 1H, J=8.5 Hz), 6.72 (d, 1H, J=2.8 Hz), 6.50 (dd, 1H, J=8.5 Hz, J=2.8 Hz), 3.91 (t, 2H, J=6.8 Hz), 3.44 (br s, 2H), 1.73-1.82 (m, 2H), 1.24-1.52 (m, 8H), 0.89 (t, 3H, J=6.8 Hz).

### 3-Chloro-4-(octyloxy)benzenamine

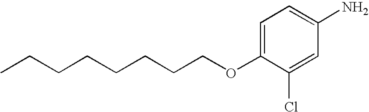

The product was obtained as a white solid in 65% (0.58 g) yield. TLC (3:1 Hex:EtOAc), R_{f}=0.5; ¹H NMR (400 MHz, CDCl₃) δ 6.74 (d, 1H, J=8.4 Hz), 6.72 (d, 1H, J=2.8 Hz), 6.51 (dd, 1H, J=8.4 Hz, J=2.8 Hz), 3.91 (t, 2H, J=6.4 Hz), 3.44 (br s, 2H), 1.73-1.81 (m, 2H), 1.23-1.51 (m, 10H), 0.88 (t, 3H, J=7.1 Hz).

### 3-Methyl-4-(octyloxy)benzenamine

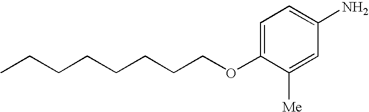

The product was obtained as a yellowish oil in 85% (0.81 g) yield. TLC (3:1 Hex:EtOAc), R_{f}=0.3; ¹H NMR (400 MHz, CDCl₃) δ 6.62 (d, 1H, J=8.4 Hz), 6.51 (d, 1H, J=2.4 Hz), 6.45 (dd, 1H, J=8.4 Hz, J=2.4 Hz), 3.85 (t, 2H, J=6.8 Hz), 3.40 (br s, 2H), 2.15 (s, 3H), 1.73-1.80 (m, 2H), 1.23-1.50 (m, 10H), 0.90 (t, 3H, J=6.8 Hz).

### Acylation of substituted alkoxy-benzenamines

To a solution of the desired substituted alkoxy-benzenamines (0.20 g, 1.0 equiv) and N-protected amino acid (1.0 equiv) in DMF (10 mL) was added DIPEA (3.0 equiv) and HATU (1.2 equiv). The reaction mixture was stirred at room temperature under an atmosphere of nitrogen 12-24 hours. The reaction was then diluted with EtOAc (25 mL) and washed with 10% NH₄Cl (2×25 mL), 5% NaHCO₃ (2×25 mL), and saturated NaCI (1×25 mL). The organic layer was dried over anhydrous MgSO₄ then the solvent removed in vacuo. The crude produce was purified using silica gel column chromatography.

### tert-Butyl (S)-2-(4-(heptyloxy)phenylcarbamoyl)-1-hydroxypropan-2-ylcarbamate:

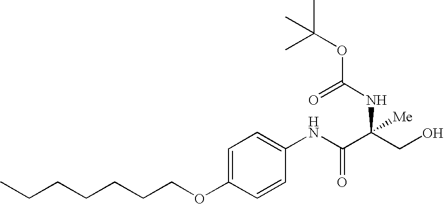

The product was obtained as a brownish solid in 78% (0.29 g) yield. TLC (1:1 EtOAc:Hex), R_{f}=0.3; ¹H NMR (400 MHz, CDCl₃) δ 9.40 (br s, 1H), 7.37 (d, 2H, J=8.8 Hz), 6.83 (d, 2H, J=8.8), 5.57 (br s, 1H), 4.02-4.12 (m, 1H), 3.91 (t, 2H, J=6.4 Hz), 3.55 (br t, 1H), 3.27 (br t, 1H), 1.71-1.80 (m, 2H), 1.55 (s, 3H), 1.46 (s, 9H), 1.23-1.50 (m, 8H), 0.89 (t, 3H, J=7.2 Hz).

### tert-Butyl (S)-2-(4-(octyloxy)phenylcarbamoyl)-1-hydroxypropan-2-ylcarbamate

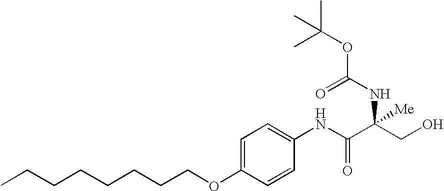

The product was obtained as a brownish solid in 49% (0.185 g) yield. TLC (1:1 EtOAc:Hex), R_{f}=0.4; ¹H NMR (400 MHz, CDCl₃) δ 9.42 (br s, 1H), 7.36 (d, 2H, J=9.0 Hz), 6.83 (d, 2H, J=9.0), 5.59 (br s, 1H), 4.03-4.13 (m, 1H), 3.91 (t, 2H, J=6.4 Hz), 3.55 (br t, 1H), 3.26 (br t, 1H), 1.71-1.80 (m, 2H), 1.56 (s, 3H), 1.46 (s, 9H), 1.23-1.50 (m, 10H), 0.88 (t, 3H, J=6.8 Hz).

### tert-Butyl (S)-2-(3-chloro-4-(heptyloxy)phenylcarbamoyl)-1-hydroxypropan-2-ylcarbamate

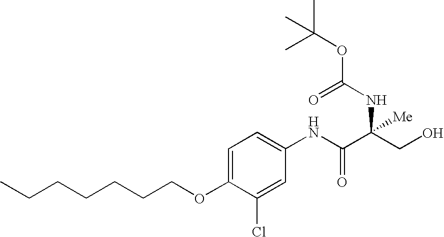

The product was obtained as an off white solid in 47% (0.169 g) yield. TLC (1:1 EtOAc:Hex), R_{f}=0.4; ¹H NMR (400 MHz, CDCl₃) δ 9.52 (br s, 1H), 7.53 (d, 1H, J=2.4 Hz), 7.28 (dd, 1H, J=8.8 Hz, J=2.4 Hz), 6.84 (d, 1H, J=8.8), 5.75 (br s, 1H), 4.02-4.10 (m, 1H), 3.98 (t, 2H, J=6.4 Hz), 3.54 (br t, 1H), 3.21 (br t, 1H), 1.76-1.85 (m, 2H), 1.55 (s, 3H), 1.46 (s, 9H), 1.24-1.51 (m, 8H), 0.89 (t, 3H, J=7.2 Hz).

### tert-Butyl (S)-2-(3-chloro-4-(octyloxy)phenylcarbamoyl)-1-hydroxypropan-2-ylcarbamate

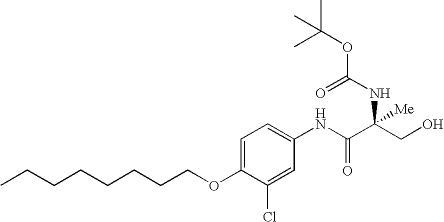

The product was obtained as a brownish solid in 40% (0.158 g) yield. TLC (1:1 EtOAc:Hex), R_{f}=0.4; ¹H NMR (400 MHz, CDCl₃) δ 9.50 (br s, 1H), 7.57 (d, 1H, J=2.4 Hz), 7.28 (dd, 1H, J=8.8 Hz, J=2.4 Hz), 6.84 (d, 1H, J=8.8), 5.58 (br s, 1H), 4.02-4.11 (m, 1 H), 3.98 (t, 2H, J=6.4 Hz), 3.54 (br t, 1H), 3.21 (br t, 1H), 1.76-1.85 (m, 2H), 1.53 (s, 3H), 1.47 (s, 9H), 1.23-1.53 (m, 10H), 0.88 (t, 3H, J=6.8 Hz).

### tert-Butyl (S)-2-(3-methyl-4-(octyloxy)phenylcarbamoyl)-1-hydroxypropan-2-ylcarbamate

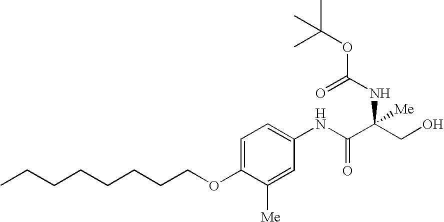

The product was obtained as an off white solid in 93% (0.133 g) yield. TLC (1:3 EtOAc:Hex), R_{f}=0.4; ¹H NMR (400 MHz, CDCl₃) δ 7.18-7.26 (m, 2H), 6.70 (d, 1H, J=8.0 Hz), 5.74 (br s, 1H), 3.94-4.08 (m, 1H), 3.89 (t, 2H, J=6.4 Hz), 3.71-3.79 (br t, 1H), 3.55-3.67 (br t, 1H), 2.19 (s, 3H), 1.72-1.82 (m, 2H), 1.55 (s, 3H), 1.45 (s, 9H), 1.22-1.52 (m, 10H), 0.89 (t, 3H, J=6.8 Hz).

Removal of Boc Protecting Group:

To a solution of the desired starting material (65 mg) in dry CH₂Cl₂ (2 mL) was added trifluoroacetic acid (TFA, 1 mL). The reaction mixture was stirred at room temperature 3-4 hours then evaporated to dryness under reduced pressure. The obtained residue was then azeotroped with CH₂Cl₂ (2×2 mL) to remove any excess TFA. The final product was either used as is or purified by reverse phase prep HPLC.

### (S)-2-Amino-N-(4-(heptyloxy)phenyl)-3-hydroxy-2-methylpropanamide

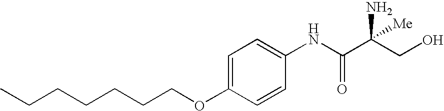

The product was obtained as a white solid in 73% (30 mg) yield. MS (ESI, M+H⁺)=309.47

### (S)-2-Amino-3-hydroxy-2-methyl-N-(4-(octyloxy)phenyl)propanamide

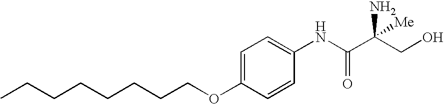

The product was obtained as a white solid in 78% (40 mg) yield. MS (ESI, M+H⁺)=323.65

### (S)-2-Amino-N-(3-chloro-4-(heptyloxy)phenyl)-3-hydroxy-2-methylpropanamide

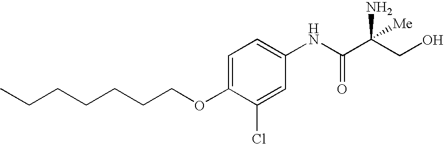

The product was obtained as a white solid in 24% (40 mg) yield. MS (ESI, M+H⁺)=343.39

### (S)-2-Amino-N-(3-chloro-4-(octyloxy)phenyl)-3-hydroxy-2-methylpropanamide

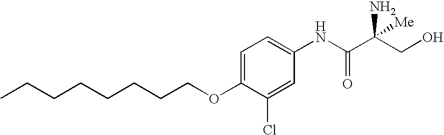

The product was obtained as a white solid in 81% (25 mg) yield. MS (ESI, M+H⁺)=357.98

### (S)-2-Amino-3-hydroxy-2-methyl-N-(3-methyl-4-(octyloxy)phenyl)propanamide

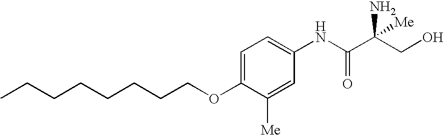

The product was obtained as a white solid in 32% (40 mg) yield. MS (ESI, M+H⁺)=337.56.

### (S)-2-(4-(Octyloxy)phenylcarbamoyl)-2-aminopropyl dihydrogen phosphate

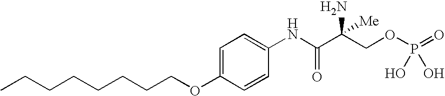

The product was obtained as white solid in 63% (24.9 mg) yield. MS (ESI, M+H⁺)=403.71; ¹H NMR (400 MHz, DMSO-d₆) δ 10.04 (s, 1H), 7.50 (d, 2H, J=8.8 Hz), 6.87 (d, 2H, J=8.8 Hz), 4.25 (dd, 1H, J=12.4 Hz, J=6.8 Hz), 4.10 (dd, 1H, J=12.8 Hz, J=6.8 Hz), 3.90 (t, 2H, J=6.4 Hz), 1.62-1.72 (m, 2H), 1.47 (s, 3H), 1.20-1.44 (m, 10H), 0.85 (t, 3H, J=7.2 Hz).

### (S)-2-(3-Fluoro-4-(octyloxy)phenylcarbamoyl)-2-aminopropyl dihydrogen phosphate

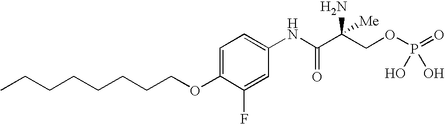

The product was obtained as white solid in 42% (2.5 mg) yield. MS (ESI, M+H⁺)=421.17; ¹H NMR (400 MHz, DMSO-d₆) δ 10.19 (s, 1H), 7.52 (dd, 1H, J=14.0 Hz, J=2.4 Hz), 7.27 (dd, 1H, J=10.0 Hz, J=1.2 Hz), 7.05 (t, 1H, J=9.6Hz), 4.20 (dd, 1H, J=11.6 Hz, J=6.4 Hz), 4.03 (dd, 1H, J=11.6 Hz, J=6.8 Hz), 3.93 (t, 2H, J=6.4 Hz), 1.59-1.68 (m, 2H), 1.41 (s, 3H), 1.14-1.38 (m, 10H), 0.80 (t, 3H, J=7.2 Hz).

### Example 2

### Synthesis of Phenylimidazole Compounds with Alkoxy Tail Group

The desired compounds were synthesized as described in Scheme 3. Substituted phenols were alkylated with the appropriate alkyl bromide using KO^{t}Bu in acetone and a catalytic amount of NaI at 50° C., or in a microwave at 80° C. using KO^{t}Bu in THF. Friedel-Crafts acylation of the corresponding phenyl ether provides the desired bromoacetophenone precursor. Reaction of the bromoacetophenone with an amino acid gave the amino acid ester as an intermediate which, upon 10 intramolecular cyclization in the presence of excess ammonium acetate, provided the desired phenylimidazole. The phenylimidazole was either deprotected to remove the Boc group using 30% TFA in CH₂Cl₂, or was phosphorylated as illustrated in Scheme 4. 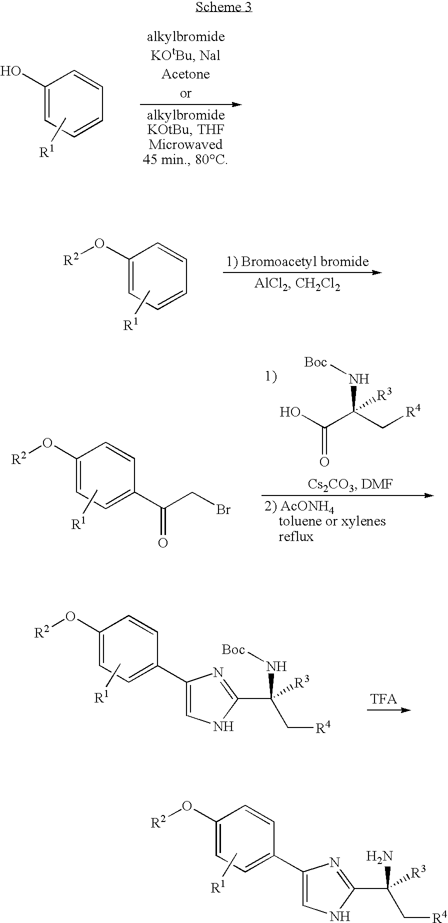
General Method for Phosphate Synthesis

This method is illustrated in Scheme 4 below. To a solution of the Boc-protected aminoalcohol (1.0 equiv) in dry CH₂Cl₂ at room temperature was added excess diethyl chlorophosphate (10-20 equiv) and triethylamine (2.5 equiv) and the reaction stirred for 12-18 hours. The crude was then loaded onto a silica gel column chromatography, as is, to purify the desired phospho-diester. The phospho-diester intermediate was reacted with excess bromotrimethylsilane (20 equiv) in dry CH₂Cl₂ at room temperature, under an atmosphere of nitrogen, over a period of 6-10 hours afforded the final phosphate which was purified by reverse-phase preparative HPLC. 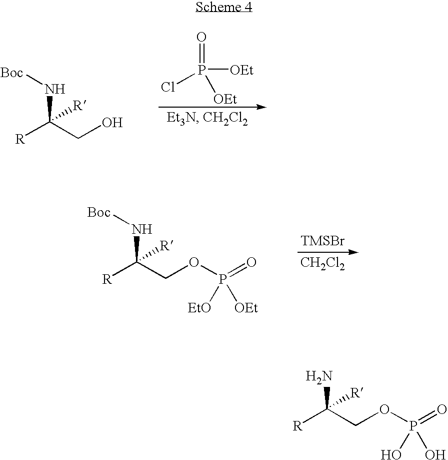
General Methods for Alkylation of Substituted Phenols

Procedure A: To a solution of desired substituted phenol (0.50 g, 1.0 equiv) and NaI (0.1 equiv) in acetone (10 mL) was added a 1.0 M solution of KO^{t}Bu in THF (1.1 equiv). To the reaction mixture is then added the desired alkyl bromide (1.1 equiv). The reaction was stirred and heated under an atmosphere of nitrogen at 50° C. for 12-24 hours. The reaction was then diluted with EtOAc (25 mL) and washed with H₂O (2×25 mL) and saturated NaCl (1×25 mL). The organic layer was dried over anhydrous MgSO₄ then the solvent removed in vacuo. The crude product was purified using silica gel column chromatography (9:1 Hex:EtOAc).

Procedure B: To a microwave tube containing the substituted phenol (0.50 g, 1.0 equiv) was added a 1.0 M solution of KO^{t}Bu in THF (1.1 equiv). To the reaction mixture was added the desired alkyl bromide (1.1 equiv). The reaction mixture was then microwaved at 80° C. for 45 minutes. The reaction was then diluted with EtOAc (25 mL) and washed with H₂O (2×25 mL) and saturated NaCl (1×25 mL). The organic layer was dried over anhydrous MgSO₄ then the solvent removed in vacuo. The crude product was purified using silica gel column chromatography (9:1 Hex:EtOAc).

### 1-(Octyloxy)benzene

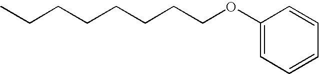

The product was obtained as an off white solid in 79% (1.0 g) yield. TLC (1:3 EtOAc:Hex), R_{f}=0.85; ¹H NMR (400 MHz, CDCl₃) δ 7.24 (m, 2H), 6.89 (m, 3H), 3.93 (t, 2H, J=6.4 Hz), 1.76-1.81 (m, 2H), 1.42-1.48 (m, 2H), 1.20-1.38 (m, 8H), 0.89 (t, 3H, J=6.8 Hz).

### 1-(Heptyloxy)benzene

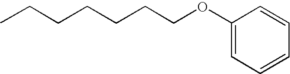

The product was obtained as brownish thick oil in 59% (0.45 g) yield. TLC (1:3 EtOAc:Hex), R_{f}=0.4; ¹H NMR (400 MHz, CDCl₃) δ 6.69-6.74 (m, 2H), 6.59-6.63 (m, 2H), 3.86 (t, 2H, J=6.9Hz), 3.41 (br s, 2H), 1.69-1.79 (m, 2H), 1.22-1.47 (m, 10H), 0.88 (t, 3H, J=7.1 Hz).

### 1-Fluoro-3-(octyloxy)benzene

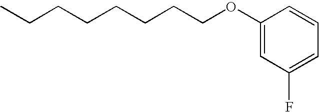

The product was obtained as a colorless oil in 84% (2.10 g) yield. TLC (1:9 EtOAc:Hex), R_{f}=0.8; ¹H NMR (400 MHz, CDCl₃) δ 7.16-7.23 (m, 1H), 6.57-6.69 (m, 3H), 3.93 (t, 2H, J=6.4 Hz), 1.73-1.82 (m, 2H), 1.23-1.50 (m, 10H), 0.89 (t, 3H, J=7.2 Hz).

### 1-Fluoro-2-(octyloxy)benzene

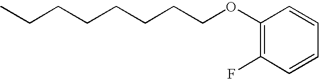

The product was obtained as a yellowish solid in 71% (0.92 g) yield. TLC (1:3 EtOAc:Hex), R_{f}=0.83; ¹H NMR (400 MHz, CDCl₃) δ 7.08-7.10 (m, 2H), 6.94 (dd, 1H), 6.80-6.88 (m, 1H), 4.02 (t, 2H, J=6.8 Hz), 1.76-1.82 (m, 2H), 1.42-1.48 (m, 2H), 1.20-1.38 (m, 8H), 0.88 (t, 3H, J=6.8 Hz).

General Method for Friedel-Crafts Acylation

To a solution of the desired phenyl ether (8.92 mmol, 1.0 equiv) in dry CH₂Cl₂ (20 mL) at −20° C. (water/salt bath) is added AlCl₃ (1.1 equiv) in portions. Bromoacetyl bromide (1.2 equiv) is then added dropwise to the reaction mixture over a period of 10-15 min. The reaction was then allowed to warm up to 0° C. or room temperature and monitored by TLC (reaction time generally 4-12 hours). The mixture was diluted with CH₂Cl₂ (50 mL), washed with H₂O (2×50 mL), and saturated NaCl (1×50 mL). The organic layer was dried over anhydrous MgSO₄ then the solvent was removed in vacuo. The crude product was purified using silica gel column chromatography (9:1 Hex:EtOAc).

### 2-Bromo-1-(4-(octyloxy)phenyl)ethanone

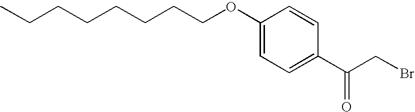

The product was obtained as an off white solid in 59% (0.461 g) yield. TLC (1:3 EtOAc:Hex), R_{f}=0.85; ¹H NMR (400 MHz, CDCl₃) δ 8.23 (d, 2H, J=6.0 Hz), 7.22 (d, 2H, J=8.0 Hz), 4.68 (s, 2H), 4.31 (t, 2H, J=6.8 Hz), 2.09 (m, 2H), 1.75 (m, 2H), 1.58 (m, 10H), 1.17 (t, 3H, J=6.8 Hz).

### 2-Bromo-1-(4-(heptyloxy)phenyl)ethanone

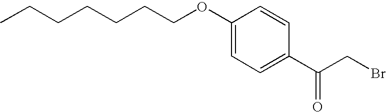

The product was obtained as an off white solid in 30% (0.93 g) yield. TLC (1:3 EtOAc:Hex), R_{f}=0.68; ¹H NMR (400 MHz, CDCl₃) δ 7.95 (d, 2H, J=7.2 Hz), 6.94 (d, 2H, J=8.8 Hz), 4.39 (s, 2H), 4.03 (t, 2H, J=6.8 Hz), 1.82 (m, 2H), 1.45 (m, 2H), 1.31 (m, 6H), 0.90 (t, 3H, J=7.2 Hz).

### 2-Bromo-1-(3-fluoro-4-(octyloxy)phenyl)ethanone

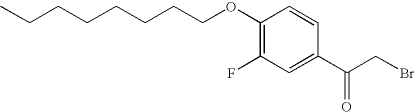

The product was obtained as a whitish solid in 39% (0.1 g). TLC (1:3 EtOAc:Hex), R_{f}=0.6; ¹H NMR (400 MHz, CDCl₃) δ 7.70-7.76 (m, 2H), 7.00 (t, 1H, J=8.0 Hz), 4.37 (s, 2H), 4.11 (t, 2H, J=6.4 Hz), 1.82-1.88 (m, 2H), 1.44-1.53 (m, 2H), 1.28-1.34 (m, 8H), 0.88 (t, 3H, J=6.8 Hz).

General Method for Imidazole Synthesis

A mixture of desired amino acid (1.0 equiv) and Cs₂CO₃ (0.5 equiv) was stirred in DMF (4 mL) for 5 minutes then to the solution was added the desired bromo-ketone (0.77 mmol, 1.0 equiv) then the mixture was stirred at room temperature for 1 hour. The reaction mixture was diluted with EtOAc (25 mL) and washed with H₂O (2×25 mL), and saturated NaCl (1×25 mL) to remove access DMF and CsBr salt. The organic layer was dried over anhydrous MgSO₄ and the solvent removed in vacuo (the DMF could also be removed either under reduced pressure without the necessity for the work-up).

To the obtained ester was then added excess (˜20 eq) ammonium acetate, and the mixture was suspended in either toluene or xylenes and refluxed for 4-6 hours under Dean-Stark conditions. The mixture was diluted with EtOAc (25 mL) and washed with H₂O (2×25 mL), and saturated NaCl (1×25 mL). The organic layer was dried over anhydrous MgSO₄ and the solvent removed in vacuo. The crude product was purified using silica gel column chromatography.

### tert-Butyl-(R)-1-hydroxy-2-(4-(4-(octyloxy)phenyl)-1H-imidazol-2-yl)propan-2-ylcarbamate

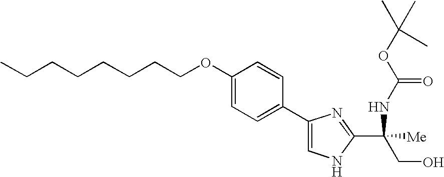

The product was obtained as a colorless foam in 35% (72 mg) yield. TLC (1:1 EtOAc:Hex), R_{f}=0.3; ¹H NMR (400 MHz, CDCl₃) δ 10.40 (br s, 1H), 7.63 (d, 2H, J==8.4 Hz), 7.10 (br s, 1H), 6.90 (d, 2H, J=8.4), 5.66 (br s, 1H), 4.85 (br s, 1H), 4.31 (d, 1H, J=11.2), 3.96 (t, 2H, J=6.8 Hz), 3.62 (d, 1H, J=11.2 Hz), 1.73-1.82 (m, 2H), 1.66 (s, 3H), 1.44 (s, 9H), 1.24-1.52 (m, 10H), 0.89 (t, 3H, J=7.2 Hz).

### tert-Butyl-(R)-2-(4-(4-(heptyloxy)phenyl)-1H-imidazol-2-yl)-1-hydroxypropan-2-ylcarbamate

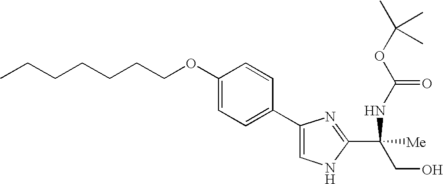

The product was obtained as a brownish solid in 17% (56 mg) yield. TLC (2:1 EtOAc:Hex), R_{f}=0.3; ¹H NMR (400 MHz, CDCl₃) δ 7.56 (d, 2H, J=8.4 Hz), 7.09 (br s, 1H), 6.90 (d, 2H, J=8.4), 5.70 (br s, 1H), 4.30 (d, 1H, J=11.2), 3.97 (t, 2H, J=6.8 Hz), 3.63 (d, 1H, J=11.2 Hz), 1.74-1.83 (m, 2H), 1.66 (s, 3H), 1.43 (s, 9H), 1.24-1.50 (m, 8H), 0.90 (t, 3H, J=7.2 Hz).

### tert-Butyl-(R)-2-(4-(2-fluoro-4-(octyloxy)phenyl)-1H-imidazol-2-yl)-1-hydroxypropan-2-ylcarbamate

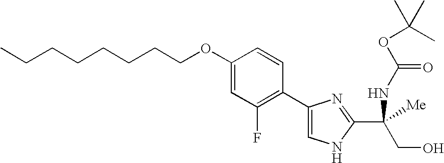

The product was obtained as a yellowish-brown solid in 20% (320 mg) yield. TLC (1:2 EtOAc:Hex), R_{f}=0.4; ¹H NMR (400 MHz, CDCl₃) δ 7.84 (br s, 1 H), 7.25 (br s, 1H), 6.73 (dd, 1H, J=12.9 Hz, J=2.4 Hz), 6.66 (dd, 1H, J=12.9 Hz, J=2.4 Hz), 5.68 (br s, 1 H), 4.31 (d, 1 H, J=11.2), 3.95 (t, 2H, J=6.4 Hz), 3.63 (d, 1H, J=11.2 Hz), 1.74-1.83 (m, 2H), 1.67 (s, 3H), 1.44 (s, 9H), 1.22-1.52 (m, 10H), 0.89 (t, 3H, J=7.0 Hz).

### tert-Butyl (R)-2-(4-(3-fluoro-4-(octyloxy)phenyl)-1H-imidazol-2-yl)-1-hydroxy propan-2-ylcarbamate

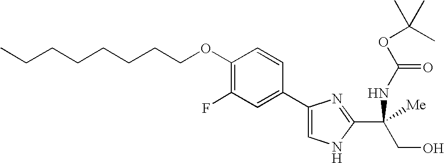

The final product was obtained as a white solid in 31% (30 mg). TLC (1:3 EtOAc:Hex), R_{f}=0.16; ¹H NMR (400 MHz, CDCl₃) δ 7.34-7.4 (m, 2H), δ 7.101 (s, 1H), 6.944 (t, 1H, J=8.4 Hz), 4.3 (d, 1H, J=11.6), 4.033 (t, 2H, J=6.8), 3.62 (d, 1H, J=11.6 Hz), 1.81-1.86 (m, 2H), 1.66 (s, 3H), 1.44-1.52 (m, 10H), 0.88 (t, 3H, J=6.8 Hz). MS (ESI, M+H⁺)=364.5

### tert-Butyl (S)-2-((benzyloxy)carbonyl)-1-(4-(4-(octyloxy)phenyl)-1H-imidazol-2-yl)ethylcarbamate

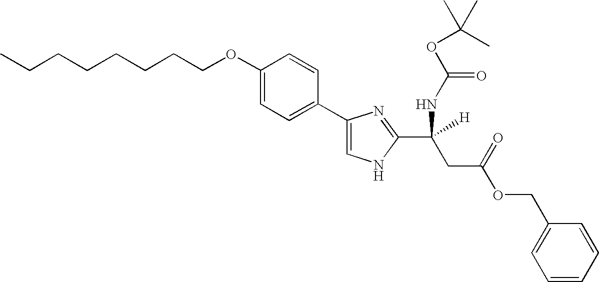

The product was obtained as a colorless oil in 64% (160 mg) yield. TLC (2:1 EtOAc:Hex), R_{f}=0.2; ¹H NMR (400 MHz, CDCl₃) δ 7.62 (br s, 1H), 7.28-7.35 (m,7H), 7.08 (s, 1H), 6.89 (d, 2H, J=9.2 Hz), 5.90 (br s, 1H), 5.08-5.21 (m, 3H), 3.97 (t, 2H, J=6.0 Hz), 3.26 (br m, 1H), 3.00 (dd, 1H, J=16.4 Hz, J=7.2 Hz), 1.74-1.84 (m, 2H), 1.46 (s, 9H), 1.23-1.54 (m, 10H), 0.89 (t, 3H, J=7.2 Hz).

General Method for Removal of Boc Protecting Group

To a solution of the desired starting material (100 mg) in CH₂Cl₂ (2 mL) was added TFA (1 mL). The reaction mixture was stirred at room temperature 2 hours then evaporated to dryness under reduced pressure. The final product was purified by reverse phase preparative HPLC.

### (R)-2-Amino-2-(4-(4-(octyloxy)phenyl)-1H-imidazol-2-yl)propan-1-ol

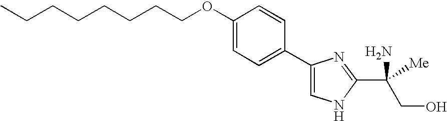

The product was obtained as a white solid in 81% (29 mg) yield. MS (ESI, M+H⁺)=346.30; ¹H NMR (400 MHz, DMSO-d₆) δ 8.38 (br s, 2H), 7.67 (d, 2H, J=8.4 Hz), 7.50 (br s, 1H), 6.92 (d, 2H, J=8.4), 5.82 (br s, 1H), 3.94 (t, 2H, J=6.4 Hz), 3.75 (d, 1H, J=11.6 Hz), 3.64 (d, 1H, J=11.6 Hz), 1.65-1.74 (m, 2H), 1.55 (s, 3H), 1.22-1.45 (m, 10H), 0.85 (t, 3H, J=7.2 Hz).

### (R)-2-Amino-2-(4-(4-(heptyloxy)phenyl)-1H-imidazol-2-yl)propan-1-ol

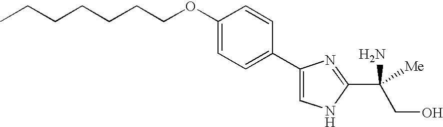

The product was obtained as a white solid in 99% (58 mg) yield. MS (ESI, M+H⁺)=332.60; ¹H NMR (400 MHz, DMSO-d₆) δ 8.34 (br s, 2H), 7.67 (d, 2H, J=8.4 Hz), 7.40 (br s, 1H), 6.92 (d, 2H, J=8.4), 5.66 (br s, 1H), 3.94 (t, 2H, J=6.8 Hz), 3.74 (d, 1H, J=11.6 Hz), 3.64 (d, 1H, J=11.6 Hz), 1.64-1.76 (m, 2H), 1.55 (s, 3H), 1.22-1.44 (m, 8H), 0.86 (t, 3H, J=7.0 Hz).

### (R)-2-Amino-2-(4-(2-fluoro-4-(octyloxy)phenyl)-1H-imidazol-2-yl)propan-1-ol

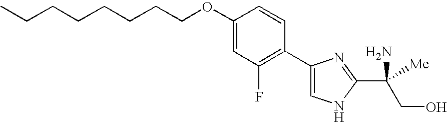

The product was obtained as a white solid in 75% (77 mg) yield. MS (ESI, M+H⁺)=364.60; ¹H NMR (400 MHz, DMSO-d₆) δ 8.40 (br s, 2H), 7.93 (br t, 1H), 7.38 (d, 2H, J=3.6 Hz), 6.81-6.70 (m, 2H), 5.67 (br s, 1H), 3.97 (t, 2H, J=6.2 Hz), 3.74 (d, 1H, J=11.6 Hz), 3.66 (d, 1H, J=11.6 Hz), 1.64-1.75 (m, 2H), 1.55 (s, 3H), 1.21-1.44 (m, 10H), 0.85 (t, 3H, J=7.2 Hz).

### (R)-2-Amino-2-(4-(3-fluoro-4-(octyloxy)phenyl)-1H-imidazol-2-yl)propan-1-ol

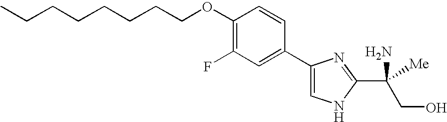

The final product was obtained as a white solid in 31% (30 mg). ¹H NMR (400 MHz, CDCl₃) δ 7.34-7.4 (m, 2H), δ 7.101 (s, 1H), 6.944 (t, 1H, J=8.4 Hz), 4.3 (d, 1H, J=11.6), 4.033 (t, 2H, J=6.8), 3.62 (d, 1H, J=11.6 Hz), 1.81-1.86 (m, 2H), 1.66 (s, 3H), 1.52-1.44 (m, 10H), 0.88 (t, 3H, J=6.8 Hz). MS (ESI, M+H⁺)=364.5

### (R)-2-Amino-2-(4-(4-(octyloxy)phenyl)-1H-imidazol-2-yl)propyl dihydrogen phosphate

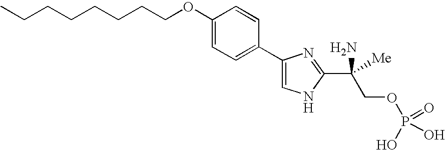

The product was obtained as white solid in 69% (22.8 mg) yield. MS (ESI, M+H⁺)=426.65; ¹H NMR (400 MHz, DMSO-d₆) δ 7.67 (d, 2H, J=8.6 Hz), 7.48 (s, 1H), 6.91 (d, 2H, J=8.6 Hz), 4.16 (dd, 1H, J=10.8 Hz, J=6.8 Hz), 4.05 (dd, 1H, J=10.8 Hz, J=6.8 Hz), 3.94 (t, 2H, J=6.8 Hz), 1.64-1.73 (m, 2H), 1.59 (s, 3H), 1.21-1.45 (m, 10H), 0.85 (t, 3H, J=7.2 Hz).

### Example 3

### Synthesis of Phenylamide Compounds with Aryl Tail Groups

Several biphenyls were synthesized using the process described in Scheme 5. Microwave assisted Suzuki cross-coupling of substituted arylboronic acids with substituted anilines afforded good to excellent yields of the biaryl amine intermediates. Furthermore, the acylation of the substituted biaryl amines with desired headpiece followed by deprotection of the Boc group afforded the final compounds. 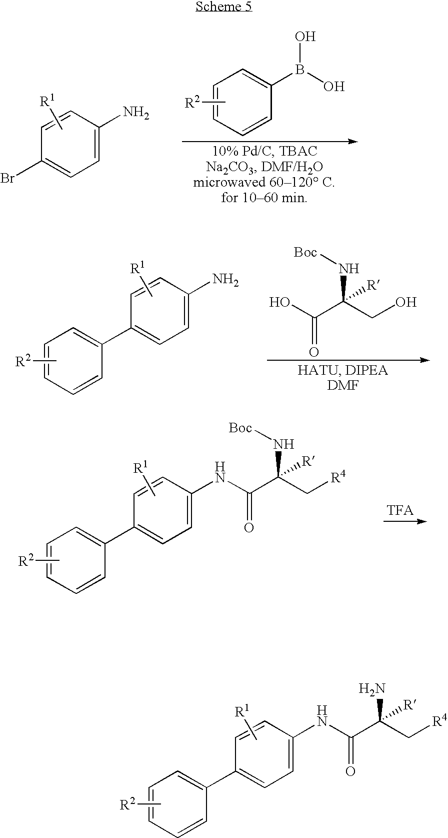
General Method for Suzuki Cross-Coupling

To a mixture of a substituted bromoaniline (1.0 equiv), substituted aryl boronic acid (1.2 equiv), 10% Pd on carbon (0.1 equiv), tetrabutylammonium chloride (0.1 equiv), and sodium carbonate (1.0 to 2.0 equiv), in a microwave tube was added a 1:1 mixture of DMF:H₂O. The mixture was then heated to 60-120° C. for 10-60 minutes using a microwave. The reaction is then diluted with EtOAc (25 mL) and washed with H₂O (2×25 mL) and saturated NaCl (1×25 mL). The organic layer was dried over anhydrous MgSO₄ and the solvent removed in vacuo. The crude product was purified using silica gel column chromatography (Hex:EtOAc) as needed.

### 4-(4-tolyl)benzenamine

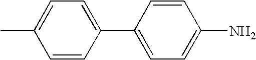

The product was obtained as a white solid in 66% (140 mg) yield. TLC (2:1 Hex:EtOAc), R_{f}=0.3; ¹H NMR (400 MHz, CDCl₃) δ 7.29-7.38 (m, 4H), 7.12 (d, 2H, J=8.4Hz), 6.67 (d, 2H, J=8.4Hz), 3.60 (br s, 2H), 2.31 (s, 3H).

### 4-(4-ethylbenzyl)benzenamine

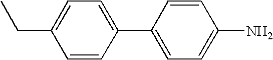

The product was obtained as a white solid in 87% (200 mg) yield. TLC (2:1 Hex:EtOAc), R_{f}=0.5; ¹H NMR (400 MHz, CDCl₃) δ 7.38-7.48 (,m, 4H), 7.24 (d, 2H, J=8.0 Hz), 6.75 (d, 2H, J=8.0 Hz), 3.40 (br s, 2H), 2.68 (q, 2H, J=7.2 Hz), 1.27 (t, 3H, J=7.2 Hz).

### 4-(benzo[d][1,3]dioxol-6-yl)benzenamine

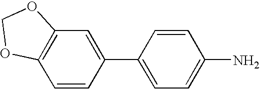

The product was obtained as a white solid in 75% (186 mg) yield. TLC (2:1 Hex:EtOAc), R_{f}=0.4; ¹H NMR (400 MHz, CDCl₃) δ 7.53-7.58 (m, 2H), 7.44-7.49 (m, 2H), 7.01-7.06 (m, 2H), 6.86 (dd, 1H, J=7.6 Hz, J=1.4 Hz), 6.00 (s, 2H), 3.40 (br s, 2H).

### tert-butyl (S)-2-(4-(4-tolyl)phenylcarbamoyl)-1-hydroxypropan-2-ylcarbamate

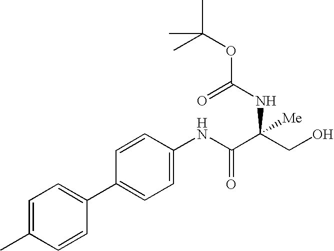

The product was obtained as a white solid in 35% (104 mg) yield. TLC (1:1 Hex:EtOAc), R_{f}=0.3; ¹H NMR (400 MHz, CDCl₃) δ 9.63 (s, 1H), 7.43-7.53 (m, 4H), 7.36-7.42 (m, 2H), 7.15-7.25 (m, 2H), 5.56 (br s, 1H), 4.05 (br s, 1H), 3.49 (br s, 1H), 3.13 (br s, 1H), 2.32 (s, 3H), 1.54 (s, 3H), 1.44 (m, 9H).

### tert-butyl (S)-2-(4-(4-ethylbenzyl)phenylcarbamoyl)-1-hydroxypropan-2-yl carbamate

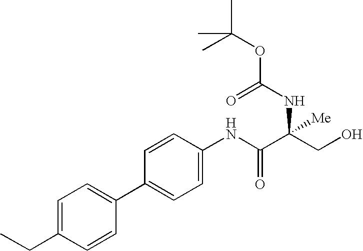

The product was obtained as a white solid in 31% (125 mg) yield. TLC (1:1 Hex:EtOAc), R_{f}=0.4; ¹H NMR (400 MHz, CDCl₃) δ 9.70 (br s, 1H), 7.42-7.64 (m, 6H), 7.24-7.32 (m, 2H), 5.62 (br s, 1H), 4.10 (br s, 1H), 3.60 (br s, 1H), 3.20 (br s, 1H), 2.70 (q, 2H, J=7.0 Hz), 1.55 (s, 3H), 1.45 (m, 9H), 1.30 (t, 3H, J=7.0 Hz).

### tert-Butyl (S)-2-(4-(benzo[d][1,3]dioxol-6-yl)phenylcarbamoyl)-1-hydroxypropan-2-ylcarbamate

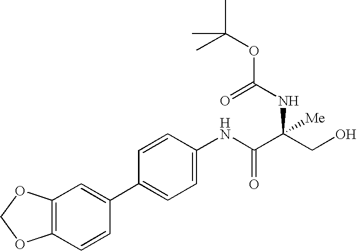

The product was obtained as a white solid in 20% (89 mg) yield. TLC (1:1 Hex:EtOAc), R_{f}=0.3; ¹H NMR (400 MHz, CDCl₃) δ 9.70 (s, 1H), 7.53-7.58 (m, 2H), 7.45-7.50 (m, 2H), 7.01-7.05 (m, 2H), 6.86 (dd, 1H, J=7.6 Hz, J=1.2 Hz), 6.00 (s, 2H), 5.62 (br s, 1H), 4.13 (br s, 1H), 3.57 (br s, 1H), 3.20 (br s, 1H), 1.55 (s, 3H), 1.48 (m, 9H).

### (S)-2-Amino-N-(4-(4-tolyl)phenyl)-3-hydroxy-2-methylpropanamide

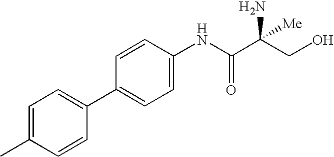

The product was obtained as a white solid in 98% (36 mg) yield. MS (ESI, M+H⁺)=285.40; ¹H NMR (400 MHz, DMSO-d₆) δ 9.94 (br s, 1H), 8.16 (br s, 2H), 7.61-7.72 (m, 4H), 7.54 (d, 2H, J=7.6 Hz), 7.24 (d, 2H, J=7.6 Hz), 5.78 (t, 1H, J=4.8 Hz), 4.00 (dd, 1H, J=11.6 Hz, J=4.8 Hz), 3.65 (dd, 1H, J=11.6 Hz, J=5.2 Hz), 2.32 (s, 3H), 1.50 (s, 3H).

### (S)-2-Amino-N-(4-(4-ethylbenzyl)phenyl)-3-hydroxy-2-methylpropanamide

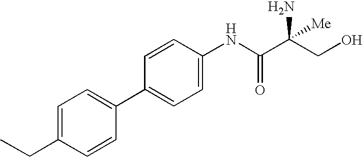

The product was obtained as a white solid in 64% (28 mg) yield. MS (ESI, M+H⁺)=299.30; ¹H NMR (400 MHz, DMSO-d₆) δ 9.95 (br s, 1H), 8.18 (br s, 2H), 7.61-7.72 (m, 4H), 7.55 (d, 2H, J=.8.2 Hz), 7.26 (d, 2H, J=8.2 Hz), 5.80 (br s, 1H), 4.00 (d, 1H, J=11.6 Hz), 3.65 (d, 1H, J=11.6 Hz), 2.61 (q, 2H, J=7.6 Hz), 1.50 (s, 3H), 1.19 (t, 3H, J=7.6Hz).

### (S)-2-amino-N-(4-(benzo[d][1,3]dioxol-6-yl)phenyl)-3-hydroxy-2-methylpropanamide

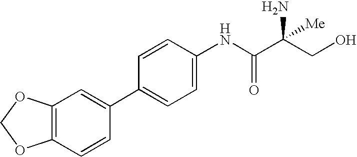

The product was obtained as a white solid in 47% (32 mg) yield. MS (ESI, M+H⁺)=315.40; ¹H NMR (400 MHz, DMSO-d₆) δ 9.93 (br s, 1H), 8.17 (br s, 2H), 7.66 (d, 2H, J=8.4 Hz), 7.59 (d, 2H, J=8.4 Hz), 7.22 (d, 1H, J=1.6 Hz), 7.12 (dd, 1H, J=6.8 Hz, J=2.0 Hz), 6.97 (d, 1H, J=8.4 Hz), 6.04 (s, 2H), 5.79 (br s, 1H), 4.00 (d, 1H, J=11.2 Hz), 3.65 (d, 1H, J=11.2 Hz), 1.50 (s, 3H).

### Example 4

### Synthesis of Substituted Biaryl Ether Compounds

General Method for the Synthesis of Substituted Biaryl Ethers

The biaryl ethers were synthesized using the general method shown in Scheme 6. To a flame dried round bottom flask is added the acylated 4-aminophenol (1 equiv. 0.15 gm), cupric acetate [Cu(OAc)₂, 1.1. equiv], desired substituted boronic acid (2.5 equiv.), and excess of 4A molecular sieves (0.6-0.9 gm). Dry dichloromethane (DCM) is then added to the reaction flask followed by the addition of anhydrous pyridine (5.0 equiv.). Oxygen is then bubbled through the reaction mixture for approximately 2 min and the reaction is stirred over night at room temperature under an atmosphere of oxygen. The following day the reaction mixture was filtered using a plug of celite to remove the molecular sieves, and the filtrate was concentrated to give a greenish solid. The crude product was purified using silica gel chromatography, EtOAc-Hexane gradient, (25% -100% EtOAc over 30 min.). The fractions corresponding to the product are pooled and the solvent removed under vacuo to give product as a white solid.

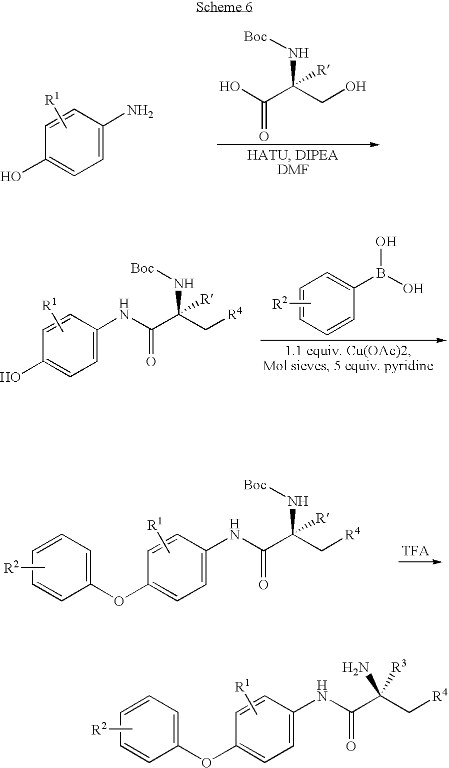

### tert-Butyl (S)-2-(4-hydroxyphenylcarbamoyl)-1-hydroxypropan-2-ylcarbamate

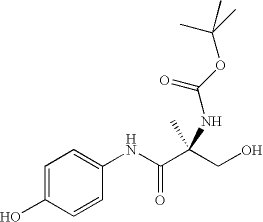

The final product was obtained as a white solid after silica gel purification using an EtOAc-Hexane gradient (15% EtOAc to 80% EtOAc over 25 min.), in 61% yield. TLC (2:1 EtOAc:Hex), R_{f} (product)=0.3; ¹H NMR (400 MHz, CDCl₃) δ 8.01 (s, 1H), 6 7.34 (d, 2H, J=8.8 Hz), 6.79 (d, 2H, J=8.8 Hz), 5.60 (br. s, 1H), 4.06 (m, 1H), 3.58 (d, 1H, J=12), 1.58 (s, 3H), 1.46 (s, 9H).

### (S)-[2-Hydroxy-1-methyl-1-(4-phenoxy-phenylcarbamoyl)-ethyl]-carbamic acid tert-butyl ester

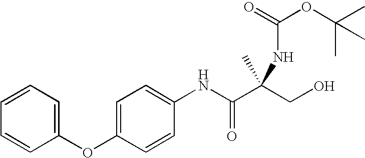

The final product was obtained as white solid following silica gel purification, in 58% yield, (0.08 g). TLC (1:1 EtOAc:Hex), R_{f}=0.2; MS (ESI, M+H⁺)=387.45; ¹H NMR (400 MHz, CDCl₃) δ 9.46 (s, 1H), 6 7.48 (d, 2H, J=8.8 Hz), 7.30 (m, 2H), 7.07 (m, 1H), 6.97 (m, 4H), 4.16 (s, 1H), 3.65 (s, 1H), 1.58 (s, 3H) 1.46 (s, 9H).

### (S)-{1-[4-(4-Ethyl-phenoxy)-phenylcarbamoyl]-2-hydroxy-1-methyl-ethyl}-carbamic acid tert-butyl ester

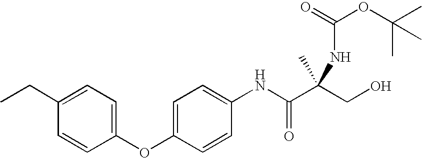

The final product was obtained as white solid following silica gel purification, in 65% yield, (0.05 g). TLC (1: 1 EtOAc:Hex), R_{f}=0.3; ¹H NMR (400 MHz, CDCl₃) δ 8.6 (s, 1H), 6 7.47 (d, 2H, J=8.0 Hz), 7.13 (d, 2H, J=8.4 Hz), 6.92 (d, 2H, J=10 Hz), 6.88 (m, 2H), 4.05 (m, 1H), 3.64 (d, 1H, J=10.8), 2.62 (q, 2H, J=16 Hz, J=8 Hz), 1.58 (s, 3H) 1.46 (s, 9H), 1.23 (t, 3H, J=7.6 Hz).

### (S)-(1-[4-(4-Butyl-phenoxy)-phenylcarbamoyl]-2-hydroxy-1-methyl-ethyl}-carbamic acid tert-butyl ester

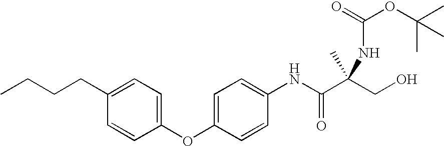

The final product was obtained as white solid following silica gel purification, in 45% yield, (0.092g). TLC (1:2 EtOAc:Hex), R_{f}=0.2; ¹H NMR (400 MHz, CDCl₃) δ 9.56 (s, 1H), 7.45 (d, 2H, J=9.2 Hz), 7.12 (d, 2H, J=8.8 Hz), 6.96 (d, 2H, J=8.8 Hz), 6.89 (d, 2H, J=8.4 Hz), 4.07 (m, 1H), 3.59 (m, 1H), 2.58 (t, 2H, J=7.6 Hz), 1.51-1.62 (m, 5H), 1.46 (s, 9H), 1.35 (m, 2H), 0.93 (t, 3H, J=7.6 Hz).

### (S)-{1-[4-(4-Butoxy-phenoxy)-phenylcarbamoyl]-2-hydroxy-1-methyl-ethyl}-carbamic acid tert-butyl ester

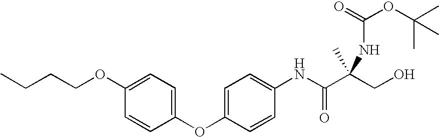

The final product was obtained as white solid following silica gel purification, in 25% yield, (0.023g). TLC (1:1 EtOAc:Hex), R_{f}=0.4; ¹H NMR (400 MHz, CDCl₃) δ 9.56 (s, 1H), 7.43 (d, 2H, J=9.2 Hz), 6.90-6.94 (m, 4H), 6.85 (d, 2H, J=9.2 Hz), 4.07 (m, 1H), 3.93 (t, 2H, J=7.6 Hz) 3.58 (m, 1H), 1.74-1.78 (m, 2H), 1.58 (s, 3H), 1.50 (m, 2H), 1.46 (s, 9H), 0.98 (t, 2H, J=7.2 Hz).

### (S)-{1-[4-(4-chloro-phenoxy)-phenylcarbamoyl]-2-hydroxy-1-methyl-ethyl}-carbamic acid tert-butyl ester

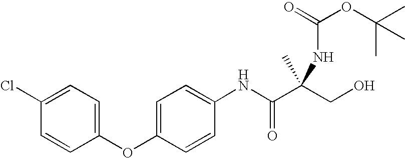

The final product was obtained as white solid following silica gel purification, in 53% yield, (0.107g). TLC (1:3 EtOAc:Hex), R_{f}=0.2; ¹H NMR (400 MHz, CDCl₃) δ 7.49 (d, 2H, J=9.2 Hz), 7.26 (d, 2H, J=8.8 Hz), 6.97 (d, 2H, J=8.8 Hz), 6.90 (d, 2H, J=8.8 Hz), 4.08 (m, 1H), 3.60 (d, 1H, J=11.2 Hz), 1.59 (s, 3H), 1.47 (s, 9H).

### (S)-{1-[4-(4-fluoro-phenoxy)-phenylcarbamoyl]-2-hydroxy-1-methyl-ethyl}-carbamic acid tert-butyl ester

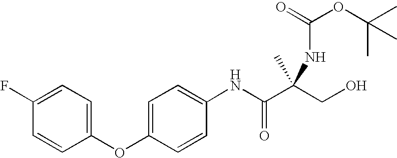

The final product was obtained as a hygroscopic solid following silica gel purification, in 33% yield, (0.063g). TLC (1:2 EtOAc:Hex), R_{f}=0.4; ¹H NMR (400 MHz, CDCl₃) δ 7.47 (d, 2H, J=9.2 Hz), 6.99 (d, 2H, J=8.0 Hz), 6.92-6.95 (m, 4H), 4.06 (m, 1H), 3.64 (d, 1H, J=10.4 Hz), 1.58 (s, 3H), 1.46 (s, 9H).

### (S)-2-Amino-3-hydroxy-2-methyl-N-(3-methyl-4-phenoxy-phenyl)-propionamide

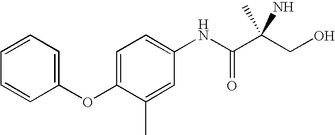

The final product was obtained as a white solid after HPLC, in 35% yield, (0.01 g). MS (ESI, M+H⁺)=301.19; ¹H NMR (400 MHz, CDCl₃) δ 9.16 (s, 1H), δ 7.25 (m, 1H), 6 7.2 (m, 3H,), 6.95 (t, 1H, J=7.6 Hz), 6.75 (d, 2H, J=8 Hz), 6.69 (d, 1H, J=7.6), 4.13 (s, 1H), 3.92 (s, 1H), 2.05 (s, 3H), 1.52 (s, 3H).

### (S)-2-Amino-3-hydroxy-N-[4-(3-methoxy-phenoxy)-phenyl]-2-methyl-propionamide

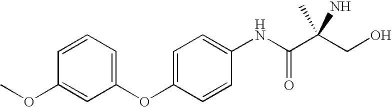

The final product was obtained as an off white solid following HPLC purification. ¹H NMR (400 MHz, DMSO-d₆) δ 9.95 (s, 1H), 8.18 (s, 2H), 7.63 (d, 2H, J=8.8 Hz), 7.24 (t, 1H, J=8.4 Hz), 7.02 (d, 2H, J=9.2 Hz), 6.68 (m, 1H), 6.52 (t, 1H, J=2.4 Hz), 6.48 (m, 1H), 3.98 (d, 1H, J=11.6 Hz), 3.71 (s, 3H), 3.64 (d, 1H, J=12.0 Hz), 1.48 (s, 3H).

### (S)-2-Amino-3-hydroxy-N-[4-(3-propoxy-phenoxy)-phenyl]-2-methyl-propionamide

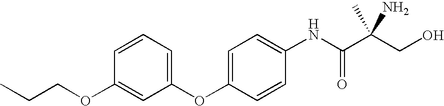

The final product was obtained as an off white solid following HPLC. ¹H NMR (400 MHz, DMSO-d₆) δ 9.93 (s, 1H), 8.14 (s, 2H), 7.63 (d, 2H, J=9.2 Hz), 7.23 (t, 1H, J=8.4 Hz), 7.02 (d, 2H, J=8.8 Hz), 6.67 (m, 1H), 6.48 (m, 2H), 5.79 (t, 1H, J=4.8 Hz), 3.98 (dd, 1H, J=4.8 and 11.6 Hz), 3.86 (t, 2H, J=6.8 Hz), 3.6 (dd, 1H, J=4.8 and 11.6 Hz), 1.68 (m, 2H ), 1.48 (s, 3H) 0.93 (t, 3H, J=7.2 Hz).

### (S)-2-Amino-3-hydroxy-N-[4-(3-isopropyl-phenoxy)-phenyl]-2-methyl-propionamide

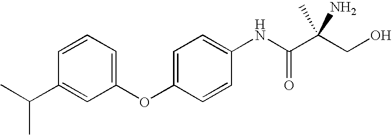

¹H NMR (400 MHz, DMSO-d₆) δ 9.93 (s, 1H), 7.63 (d, 2H, J=8.8 Hz), 7.28 (t, 1H, J=8 Hz), 7.03 (m, 1H), 7.01 (m, 2H), 6.87 (t, 1H, J=2.0 Hz), 6.74 (m, 1H), 3.98 (dd, 1H, J=4.4 and 11.2 Hz), 3.62 (dd, 1H, J=4.4 and 11.6 Hz), 3.09 (q, 1H, J=7.6 and 14.8 Hz), 2.87 (m, 1H), 1.47 (s, 3H) 1.18 (d, 6H, J=6.0 Hz).

### (S)-2-Amino-3-hydroxy-N-[4-(3-trifluoromethyl-phenoxy)-phenyl]-2-methyl-propionamide

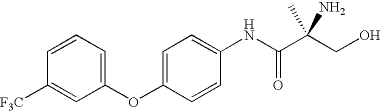

¹H NMR (400 MHz, DMSO-d₆) δ 9.98 (s, 1H), 7.70 (d, 2H, J=9.2 Hz), 7.47 (m, 1H), 7.26 (m, 2H), 7.14 (d, 2H, J=9.2 Hz), 4.01 (dd, 1H, J=4.0 and 11.2 Hz), 3.66 (dd, 1H, J=4.0 and 11.6 Hz), 1.51 (s, 3H).

### (S)-2-Amino-N-[4-(3-benzyloxy-phenoxy)-phenyl]-3-hydroxy-2-methyl-propionamide

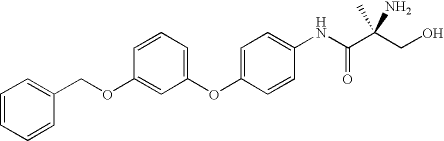

¹H NMR (400 MHz, DMSO-d₆) δ 9.93 (s, 1H), 8.17 (s, 2H), 7.64 (d, 2H, J=9.2 Hz), 7.41 (m, 2H), 7.38 (m, 1H), 7.35 (m, 1H), 7.27 (t, 1H, J=8.0 Hz), 7.04 (d, 2H, J=9.2 Hz), 6.78 (m, 1H), 6.61 (t, 1H, J=2.4 Hz), 6.52 (m, 1H), 5.80 (t, 1H, J=4.8 Hz), 5.08 (s, 2H), 4.00 (dd, 1H, J=4.4 and 11.2 Hz), 3.65 (dd, 1H, J=4.8 and 11.2 Hz), 1.49 (s, 3H).

### (S)-2-Amino-3-hydroxy-N-14-(3-isopropoxy-phenoxy)-phenyl]-2-methyl-propionamide

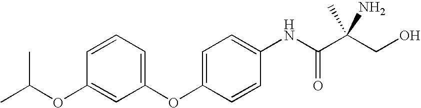

¹H NMR (400 MHz, DMSO-d₆) δ 8.15 (s, 2H), 7.62 (d, 2H, J=9.2 Hz), 7.22 (t, 1H, J=8.8 Hz), 7.03 (d, 2H, J=8.8 Hz), 6.65 (m, 1H), 6.47 (m, 2H), 5.76 (t, 1H, J=4.4 Hz), 4.55 (m, 1H), 3.98 (dd, 1H, J=5.2 and 12.0 Hz), 3.62 (dd, 1H, J=4.8 and 12.0 Hz), 1.47 (s, 3H), 1.23 (s, 3H), 1.21 (s, 3H).

### (S)-2-Amino-N-[4-(3-butoxy-phenoxy)-phenyl]-3-hydroxy-2-methyl-propionamide

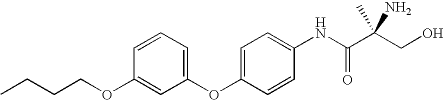

¹H NMR (400 MHz, DMSO-d₆) δ 7.62 (d, 2H, J=9.2 Hz), 7.23 (t, 1H, J=8.4 Hz), 7.03 (d, 2H, J=9.2 Hz), 6.66 (m, 1H), 6.48 (m, 2H), 5.75 (t, 1H, J=4.4 Hz), 3.97 (dd, 1H, J=5.2 and 11.2 Hz), 3.93 (t, 2H, J=9.2 Hz), 3.62 (dd, 1H, J=5.2 and [1.6 Hz), 1.65 (m, 2H), 1.47 (s, 3H), 1.39 (m, 2H), 0.90 (t, 3H, J=7.2 Hz).

### (S)-2-Amino-N-[4-(3-ethoxy-phenoxy)-phenyl]-3-hydroxy-2-methyl-propionamide

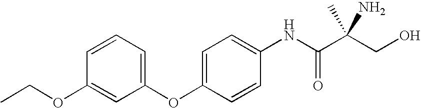

¹H NMR (400 MHz, DMSO-d₆) δ 7.62 (d, 2H, J=8.8 Hz), 7.24 (t, 1H, J=8.8 Hz), 7.03 (d, 2H, J=9.2 Hz), 6.66 (m, 1H), 6.49 (m, 2H), 5.77 (t, 1H, J=4.4 Hz), 3.96 (m, 3H), 3.63 (dd, 1H, J=5.2 and 12.0 Hz), 1.467 (s, 3H), 1.28 (t, 3H, J=7.2 Hz).

### Example 5

### Synthesis of Phenylamide Compounds with Arylalkoxy and Cycloalkylalkoxy Tail Groups

### (A) (S)-2-amino-3-hydroxy-2-methyl-N-(4-(biphenethyloxy)phenyl)propanamide trifluoroacetic acid salt

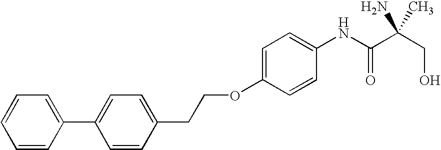

### 1-(2-(4-nitrophenoxy)ethyl)biphenyl

2-biphenyl ethanol (1 g, 5 mmol), 4-nitrophenol (834 mg, 6 mmol), and triphenylphosphine (1.59 g, 6 mmol) was dissolved in 20 mL dichloromethane. The solution was chilled in an ice-water bath prior to the addition of diethylazodicarboxylate (949 μl, 6 mmol). The reaction was then stirred overnight, and the ice-water bath slowly warmed to room temperature. Crude product was purified by flash chromatography to yield 640 mg crystalline solid. ¹H NMR (400 MHz, DMSO-d₆) δ 8.1 (d), 7.6 (m), 7.47-7.41 (m), 7.34 (m), 7.17 (m), 4.39 (t, 2H), 3.13 (t, 2H).

### tert-butyl (S)-2-(4-(phenethyloxy)biphenylcarbamoyl)-1-hydroxypropan-2-ylcarbamate

1-(2-(4-nitrophenoxy)ethyl)biphenyl (300 mg, 0.94 mmol) was dissolved in a mixture of absolute ethanol and ethyl acetate. The mixture was purged with nitrogen gas prior to the addition of 150 mg 10% Pd on carbon. The reaction was capped with a septum and stirred under 1 atm H₂ (g) overnight. Reaction was judged complete by TLC (R_{f} product ˜0.5 in 1:1 EtOAc:hexanes). The solution was filtered through celite and the solvent evaporated under vacuum. Without further purification, the crude product was combined with N-(Boc)-α-methylserine (210 mg), HATU (364 mg), DIPEA (416 μl), and 10 mL DMF. The solution was stirred at room temperature for 3 hours. Solvent was removed by rotary evaporator and crude product purified by flash chromatography to yield 240 mg yellow liquid, 52% yield.

### 2-amino-3-hydroxy-2-methyl-N-(4-(phenethyloxy)biphenyl)propanamide trifluoroacetic acid salt

tert-butyl(S)-2-(4-(phenethyloxy)biphenylcarbamoyl)-1-hydroxypropan-2-ylcarbamate (80 mg) was dissolved in a 1:1 mixture of 2 mL DCM:TFA for 3 hours. The title compound was purified by reverse phase chromatography and 29.6 mg white solid isolated as the TFA salt (in some cases reverse phase purification was not necessary). MS (ESI, M+H⁺)=391.2; ¹H NMR (400 MHz, DMSO-d₆) δ 7.65 (m), 7.60 (m), 7.52-7.40 (m), 6.93 (m), 4.197 (t, 2H), 3.8 (bm, 1H), 3.5 (bm, 1H), 3.06 (t, 2H), 1.38 (s, 3H).

### (B) (S)-2-(4-(biphenethyloxy)phenylcarbamoyl)-2-aminopropyl dihydrogen phosphate

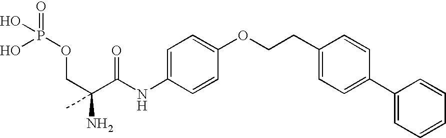

### tert-butyl (S)-2-(4-(phenethyloxy)phenylcarbamoyl)-1-diethylphosphatidylpropan-2-ylcarbamate

2-amino-3-hydroxy-2-methyl-N-(4-(phenethyloxy)biphenyl)propanamide trifluoroacetic acid salt (116 mg), diethylchloridophosphite (171 μl, 5 eq), and DIPEA (8 eq) were combined in 2 ml anhydrous DCM under N₂ atmosphere. After 8 hours conversion to product remained low, ˜20%, as judged by TLC (R_{f}˜0.2 in 80% EtOAc:hexanes). More diethylchloridophosphite (171 μl, 5 eq) and DIPEA (8 eq) were added to the reaction mixture and the solution stirred overnight. The next morning TLC showed ˜100% conversion to product. Flash chromatography yielded 10 mg of pure product (20% yield). MS (ESI, M+Na⁺)=649.

### (S)-2-(4-(biphenethyloxy)phenylcarbamoyl)-2-aminopropyl dihydrogen phosphate.

(S)-2-(4-(phenethyloxy)phenylcarbamoyl)-2-aminopropyl diethyl phosphate (10 mg) was dissolved in 3 ml DCM, immersed in an ice bath, and excess trimethylsilylbromide added (20 eq). The reaction was monitored by liquid chromatography/mass spectrometry (LCMS). Complete disappearance of the starting material occurred overnight while stirring at room temperature. Solvent was evaporated and the crude product purified by reverse phase chromatography to yield 1.5 mg of the title compound (16% yield). MS (ESI, M+H⁺)=471.1; ¹H NMR (400 MHz, DMSO-d₆) δ 7.66-7.63 (m), 7.60-7.40 (m), 7.36 (m), 4.3 (m, 1H), 4.20 (t, 2H), 4.05 (bm, 1H), 3.051 (t, 2H), 1.48 (s).

### (C) (S)—N-(4-(4-(thiophen-2-yl)butoxy)phenyl)-2-amino-3-hydroxy-2-methylpropanamide trifluoroacetic acid salt

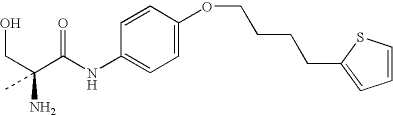

### tert-butyl (S)-2-(4-(4-(thiophen-2-yl)butoxy)phenylcarbamoyl)-1-hydroxypropan-2-ylcarbamate

This compound was synthesized from 2-(4-(4-nitrophenoxy)butyl)thiophene (280 mg), N-(Boc)-α-methylserine (205 mg), HATU (442 mg), and DIPEA (506 μl) following the procedure described in Example 5(A) to yield 280 mg product (62% yield). ¹H NMR (400 MHz, DMSO-d₆) δ 9.24 (s, 1H), 7.44 (m, 2H), 7.29 (m, 1H), 6.9 (m, 1H), 6.84-6.81 (m, 3H), 5.00 (t, 1H), 3.93 (t, 2H), 3.61 (m, 2H), 2.84 (t, 2H), 1.73 (m, 4H), 1.48 (overlapping singlets, 9H).

### (S)—N-(4-(4-(thiophen-2-yl)butoxy)phenyl)-2-amino-3-hydroxy-2-methylpropanamide trifluoroacetic acid salt.

This compound was synthesized from tert-butyl (S)-2-(4-(4-(thiophen-2-yl)butoxy)phenylcarbamoyl)-1-hydroxypropan-2-ylcarbamate (140 mg) according to the procedure provided in Example 5(A) to yield 31 mg white solid title compound. MS (ESI, M+H⁺)=349.5; ¹H NMR (400 MHz, DMSO-d₆) δ 9.79 (bs, 1H), 7.48 (m, 2H), 7.29 (m, 1H), 6.9-6.8 (m, 5H), 5.6 (bs, 1H), 3.95+3.8 (overlapping signals, 3H), 3.55 (m, 1H), 2.84 (m, 2H), 1.73 (m, 4H), 1.41 (s, 3H).

### (D) (S)-2-(4-(4-(thiophen-2-yl)butoxy)phenylcarbamoyl)-2-aminopropyl dihydrogen phosphate

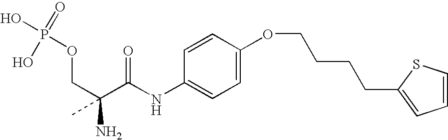

(S)—N-(4-(4-(thiophen-2-yl)butoxy)phenyl)-2-amino-3-hydroxy-2-methylpropanamide trifluoroacetic acid salt (116 mg), di(tert-butyl) diisopropylamidophosphite (143 mg, 163 μl), and 1H-tetrazole (108 mg) were combined in 3 ml anhydrous THF under N₂ (g) and stirred overnight. LCMS showed leftover starting material and more di(tert-butyl) diisopropylamidophosphite (143 mg, 163 μl), and 1H-tetrazole (108 mg) were added to the reaction mixture. The reaction was complete after several days stirring at room temperature. 264 ul of 30% aq H₂O₂ was then added to the solution and the reaction stirred for an additional 2.5 hours prior to quenching with 1 mL saturated sodium thiosulfite soln. The resulting mixture was diluted with EtOAc and the organic layer collected, concentrated, and purified by flash chromatography yielding 45 mg tert-butyl (S)-2-(4-(4-(thiophen-2-yl)butoxy)phenylcarbamoyl)-1-di-tert-butyl phosphatidylpropan-2-ylcarbamate. The purified sample was then dissolved in 2 mL 25% TFA:DCM and stirred for 1 hour. The solution was concentrated to yield 24 mg of the title compound. MS (ESI, M+H⁺)=429.2; ¹H NMR (400 MHz, DMSO-d₆) δ 9.8 (bs, 1H), 7.48 (m, 2H), 7.29 (m, 1H), 6.9-6.8 (m, 5H), 4.25 (m, 1H), 4.05 (m, 1H), 3.95 (bt, 3H), 3.55 (m, 1H), 2.84 (m, 2H), 1.73 (m, 4H), 1.41 (s, 3H).

### (E) (S)—N-(4-(4-(4-methoxyphenyl)butoxy)phenyl)-2-amino-3-hydroxy-2-methylpropanamide trifluoroacetic acid salt

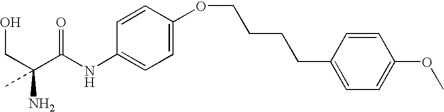

1-(4-(4-nitrophenoxy)butyl)-4-methoxybenzene (470 mg) was converted to 305 mg tert-butyl (S)-2-(4-(4-(4-methoxyphenyl)butoxy)phenylcarbamoyl)-1-hydroxypropan-2-ylcarbamate following the general procedure provided in Example 5(A) employing N-(Boc)-α-methylserine (210 mg), HATU (360 mg), and DIPEA (860 ul). MS (ESI, M+Na⁺)=495.7. The carbamate (130 mg) was deprotected following the procedure in Example 5(A) yielding 108 mg of the title compound. MS (ESI, M+H+)=373.9.

(F) (S)-2-(4-(4-(4-methoxyphenyl)butoxy)phenylcarbamoyl)-2-aminopropyl dihydrogen phosphate

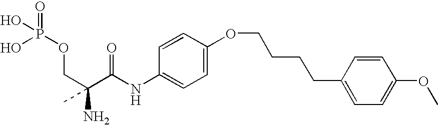

This compound was synthesized from (S)—N-(4-(4-(4-methoxyphenyl)butoxy)phenyl)-2-amino-3-hydroxy-2-methylpropanamide trifluoroacetic acid salt (115 mg) as described in Example 5(D) to yield 23 mg solid product. ¹H NMR (400 MHz, DMSO-d₆) δ 9.9 (bs, 1H), 7.5 (d, 2H), 7.16 (d, 2H), 6.87 (d, 2H), 6.82 (d, 2H), 4.21 (m, 1H), 4.11 (m, 1H), 3.92 (m, 3H), 3.75 (s, 3H), 2.55 (m, 2H), 1.65 (m, 4H), 1.42 (s, 3H).

### (G) (S)—N-(4-(3-(trifluoromethyl)phenethyloxy)phenyl)-2-amino-3-hydroxy-2-methylpropanamide trifluoroacetic acid salt

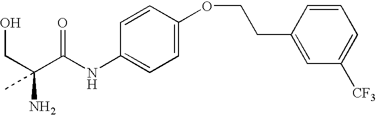

1-(3-(trifluoromethyl)phenethyloxy)-4-nitrobenzene (470 mg) was converted to 290 mg tert-butyl (S)-2-(4-(3-(trifluoromethyl)phenethyloxy)phenylcarbamoyl)-1-hydroxypropan-2-ylcarbamate following the general procedure provided in Example 5(A) employing N-(Boc)-a-methylserine (210 mg), HATU (360 mg), and DIPEA (900 μl). MS (ESI, M+H⁺)=483.4. The carbamate (145 mg) was deprotected following the procedure in Example 5(A) yielding 143 mg of the title compound. MS (ESI, M+H⁺)=383.1. ¹H NMR (400 MHz, DMSO-d₆) δ 9.8 (bs, 1H), 7.67-7.40 (m, 6H), 6.88 (m, 2H), 5.67 (bs, 1H), 4.18 (t, 3H), 3.91 (m, 1H), 3.58 (m, 1H), 3.11 (t, 3H), 1.41 (s, 3H).

### (H) (S)-2-(4-(3-(trifluoromethyl)phenethyloxy)phenylcarbamoyl)-2-aminopropyl dihydrogen phosphate

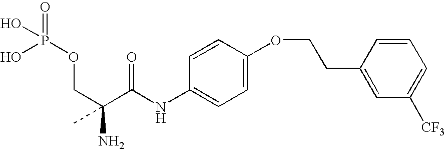

This compound was synthesized from (S)—N-(4-(3-(trifluoromethyl)phenethyloxy)phenyl)-2-amino-3-hydroxy-2-methylpropanamide trifluoroacetic acid salt (124 mg) as described for Example 5(D) to yield 50 mg solid product. MS (ESI, M+H⁺)=463.1; ¹H NMR (400 MHz, DMSO-d₆) δ 9.95 (bs, 1H), 7.68-7.49 (m, 6H), 6.90 (m, 2H), 4.18 (t, 3H), 4.05 (m, 2H), 3.11 (t, 3H), 1.41 (s, 3H).

### (I) (S)—N-(4-(4-phenylbutoxy)phenyl)-2-amino-3-hydroxy-2-methylpropanamide trifluoroacetic acid salt

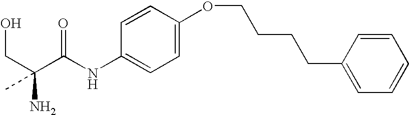

1-(4-phenylbutoxy)-4-nitrobenzene (730 mg) was converted to 305 mg tert-butyl (S)-2-(4-(4-phenylbutoxy)phenylcarbamoyl)-1-hydroxypropan-2-ylcarbamate following the general procedure provided in Example 5(A) employing N-(Boc)-α-methylserine (210 mg), HATU (360 mg), and DIPEA (860 μl). MS (ESI, M+Na⁺)=465.5. The carbamate (152 mg) was deprotected following the procedure in AS 1-C yielding 111 mg of the title compound. MS (ESI, M+H⁺)=343.9.

### (J) (S)-2-(4-(4-phenylbutoxy)phenylcarbamoyl)-2-aminopropyl dihydrogen phosphate

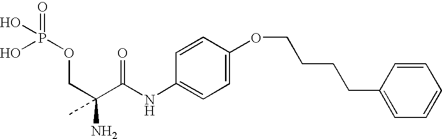

This compound was synthesized from (S)—N-(4-(4-phenylbutoxy)phenyl)-2-amino-3-hydroxy-2-methylpropanamide trifluoroacetic acid salt (120 mg) in a manner similar to that provided in Example 5(D) to yield 40 mg solid product. MS (ESI, M+H⁺)=423.7; ¹H NMR (400 MHz, DMSO-d₆) δ 9.95 (s, 1H), 7.47 (d, 6H), 7.27-7.13 (m, 5H), 6.88 (m, 2H), 4.21 (t, 1H), 4.06 (m, 1H), 3.94 (t, 2H), 2.64 (m, 2H), 1.7 (m, 4H), 1.44 (s, 3H).

### (K) (S)—N-(4-(5-phenylpentyloxy)phenyl)-2-amino-3-hydroxy-2-methylpropanamide trifluoroacetic acid salt

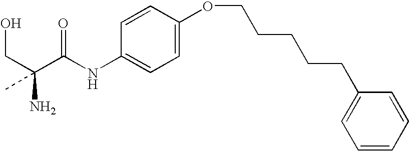

1-(5-phenylpentyloxy)-4-nitrobenzene (560 mg) was converted to 260 mg tert-butyl (S)-2-(4-(5-phenylpentyloxy)phenylcarbamoyl)-1-hydroxypropan-2-ylcarbamate following the general procedure outlined in Example 5(A) employing N-(Boc)-α-methylserine (210 mg), HATU (360 mg), and DIPEA (860 μl). MS (ESI, M+Na⁺)=357.8. The carbamate (150 mg) was deprotected following the procedure in Example 5(A) yielding 147 mg of the title compound. MS (ESI, M+H⁺)=357.8.

### (L) (S)-2-(4-(5-phenylpentyloxy)phenylcarbamoyl)-2-aminopropyl dihydrogen phosphate

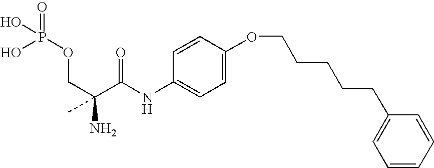

This compound was synthesized from (S)—N-(4-(5-phenylpentyloxy)phenyl)-2-amino-3-hydroxy-2-methylpropanamide (117 mg) as described in Example 5(D) to yield 66 mg solid product. MS (ESI, M+H⁺)=437.5; ¹H NMR (400 MHz, DMSO-d₆) δ 9.95 (s, 1H), 7.48 (m, 2H), 7.23 (m, 2H), 7.16 (m, 2H), 6.88 (m, 2H), 4.27 (t, 1H), 4.07 (m, 1H), 3.92 (t, 2H), 2.57 (t, 2H), 1.7 (m, 2H), 1.65 (m, 2H), 1.5 (s, 3H), 1.42 (m, 2H).

### (M) (S)—N-(4-(4-cyclohexylbutoxy)phenyl)-2-amino-3-hydroxy-2-methylpropanamide trifluoroacetic acid salt

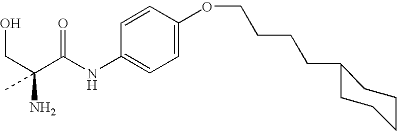

1-(4-cyclohexylbutoxy)-4-nitrobenzene (1 g) was converted to 260 mg tert-butyl (S)-2-(4-(4-cyclohexylbutoxy)phenylcarbamoyl)-1-hydroxypropan-2-ylcarbamate following the general procedure outlined in Example 5(A) employing N-(Boc)-α-methylserine (210 mg), HATU (360 mg), and DIPEA (860 μl). MS (ESI, M+Na⁺)=449. The carbamate (87 mg) was deprotected following the procedure in Example 5(A) yielding 81 mg of the title compound. MS (ESI, M+H⁺)=349.5. ¹H NMR (400 MHz, DMSO-d₆) δ 9.73 (s, 1H), 7.47 (m, 2H), 6.88 (m, 2H), 5.65 (m, 1H), 4.27 (overlapping signals, 3H), 3.6 (m, 1H), 1.6 (m, 6H), 1.4 (m, 5H), 1.15 (m, 6H), 0.85 (m, 3H).

### (N) (S)-2-(4-(4-cyclohexylbutoxy)phenylcarbamoyl)-2-aminopropyl dihydrogen phosphate

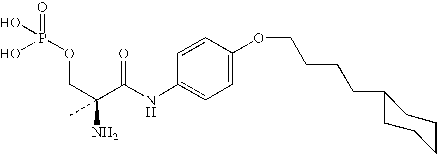

This compound was synthesized from (S)—N-(4-(4-cyclohexylbutoxy)phenyl)-2-amino-3-hydroxy-2-methylpropanamide trifluoroacetic acid salt (173 mg) as described in Example 5(D) to yield 27 mg solid product. MS (ESI, M+H⁺)=429; ¹H NMR (400 MHz, DMSO-d₆) δ 9.8 (bs, 1H), 7.47 (m, 2H), 6.88 (m, 2H), 4.15 (m, 1H), 4.02 (m, 1H), 3.90 (t, 2H), 1.68 (m, 6H), 1.4 (m, 5H), 1.15 (m, 6H), 0.85 (m, 3H).

### Example 6

### Synthesis of Carboxylic Acid Compounds

General method for acylation of substituted 4-aminophenol

To a solution of N-(Boc)-α-methylserine (1.0 equiv) in DMF (10 mL) was added DIPEA (3.0 equiv) and HATU (1.2 equiv), followed by the addition of 4-aminophenol (1.0 equiv.). The reaction mixture was stirred at room temperature under an atmosphere of nitrogen for 12-24 hours. The reaction was then diluted with EtOAc (25 mL) and washed with 10% NH₄Cl (2×25 mL), 5% NaHCO₃ (2×25 mL), and saturated NaCl (1×25 mL). The organic layer was dried over anhydrous MgSO₄ then the solvent removed in vacuo. The crude product was purified using silica gel column chromatography.

### tert-Butyl (S)-2-((benzyloxy)carbonyl)-1-(4 (octyloxy)phenyl-carbamoyl)ethylcarbamate

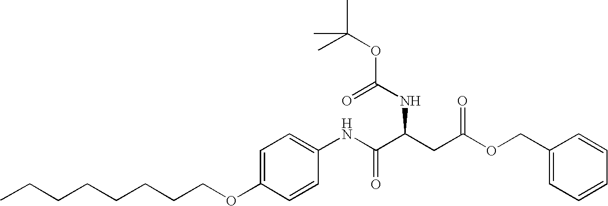

The product was obtained as a yellow solid in 94% (2.34 g) yield. TLC (1:2 EtOAc:Hex), R_{f}=0.6; ¹H NMR (400 MHz, CDCl₃) δ 8.31 (br s, 1H), 7.30-7.38 (m, 7H), 6.83 (d, 2H, J=9.0 Hz), 5.80 (br s, 1H), 5.18 (d, 1H, J=12.5 Hz), 5.13 (d, 1H, J=12.5 Hz), 4.62 (br s, 1H), 3.92 (t, 2H, J=6.6 Hz), 3.05-3.13 (m, 1H), 2.75-2.83 (m, 1H), 1.72-1.81 (m, 2H), 1.23-1.50 (m, 10H), 1.47 (s, 9H), 0.89 (t, 3H, J=7.0 Hz).

### tert-Butyl (S)-3-((benzyloxy)carbonyl)-1-(4-(octyloxy)phenylcarbamoyl)propylcarbamate

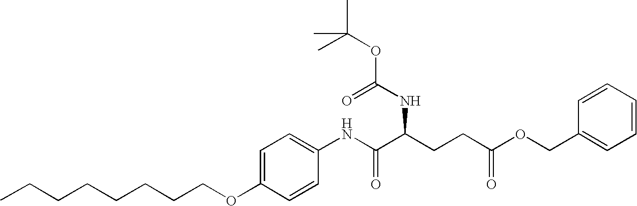

The product was obtained as a yellow solid in 94% (2.28 g) yield. TLC (1:2 EtOAc:Hex), R_{f}=0.6; ¹H NMR (400 MHz, CDCl₃) δ 8.43 (br s, 1 H), 7.31-7.40 (m, 7H), 6.84 (d, 2H, J=8.9 Hz), 5.30 (br s, 1H), 5.10-5.19 (m, 2H), 4.25 (br s, 1H), 3.92 (t, 2H, J=6.7 Hz), 2.60-2.70 (m, 1H), 2.45-2.56 (m, 1H), 2.13-2.28 (m, 1H), 1.95-2.06 (m, 1H), 1.72-1.80 (m, 2H), 1.23-1.48 (m, 10H), 1.45 (s, 9H), 0.89 (t, 3H, J=6.9 Hz).

General method for deprotection of Cbz-amino acids

To a solution of Boc-protected amino acid ester (1.0 equiv) in MeOH at room temperature was added 10% Pd on carbon (0.1 equiv by mass) and stirred under H₂ atmosphere for 6-18 hours. The solution was then filtered through Celite to remove Pd and Carbon. The filtrated was evaporated to dryness. The residue was then dissolved in CH₂Cl₂ and TFA (2:1) and stirred at room temperature 2 hours to remove the Boc protecting group. The solvent was then evaporated to dryness under reduced pressure. The final product was purified by prep HPLC as necessary.

### (S)-3-amino-3-(4-(4-(octyloxy)phenyl)-1H-imidazol-2-yl)propanoic acid

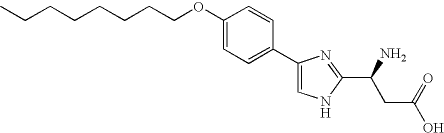

The product was obtained as a white solid in 95% (65 mg) yield. MS (ESI, M+H⁺)=360.17; ¹H NMR (400 MHz, DMSO-d₆) δ 8.42 (br s, 3H), 7.64 (d, 2H, J=8.8 Hz), 7.48 (s, 1H), 6.93 (d, 2H, J=8.8), 4.64 (br t, 1H, J=6.4 Hz), 3.94 (t, 2H, J=6.8 Hz), 3.12 (dd, 1H, J=17.2 Hz, J=6.8 Hz), 2.94 (dd, 1H, J=17.2 Hz, J=6.8 Hz), 1.64-1.75 (m, 2H), 1.20-1.45 (m, 10H), 0.85 (t, 3H, J=7.2 Hz).

### (S)-3-(4-(octyloxy)phenylcarbamoyl)-3-aminopropanoic acid

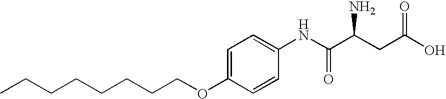

The product was obtained as a white solid in 99% (175 mg) yield. MS (ESI, M+H⁺)=337.36; ¹H NMR (400 MHz, DMSO-d₆) δ 10.30 (br s, 1H), 8.26 (br s, 3H), 7.45 (d, 2H, J=9.0 Hz), 6.88 (d, 2H, J=9.0 Hz), 4.18-4.24 (br s, 1H), 3.90 (t, 2H, J=6.3 Hz), 2.74-2.98 (m, 2H), 1.60-1.76 (m, 2H), 1.16-1.45 (m, 10H), 0.85 (t, 3H, J=7.0 Hz).

### (S)-4-(4-(octyloxy)phenylcarbamoyl)-4-aminobutanoic acid

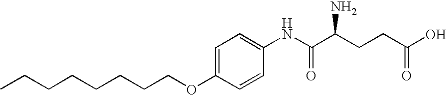

The product was obtained as a white solid in 99% (150 mg) yield. MS (ESI, M+H⁺)=351.40; ¹H NMR (400 MHz, DMSO-d₆) δ 10.30 (br s, 1H), 7.45 (d, 2H, J=9.2 Hz), 6.89 (d, 2H, J=9.2 Hz), 3.85-3.95 (m, 3H), 2.35 (t, 2H, J=7.0 Hz), 1.96-2.06 (m, 2H), 1.62-1.72 (m, 2H), 1.18-1.43 (m, 10H), 0.84 (t, 3H, J=7.0 Hz).

### (S)-2-amino-N⁵-hydroxy-N¹-(4-(octyloxy)phenyl)pentanediamide

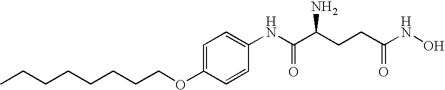

The Boc-protected carboxylate intermediate from previous step was coupled with hydroxylamine hydrochloride using general HATU coupling conditions. After TFA deprotection of Boc group the final compound was purified by prep HPLC as a white solid in 20% (12 mg) yield. MS (ESI, M+H⁺)=366.48; ¹H NMR (400 MHz, DMSO-d₆) δ 10.53 (br s, 0.5H), 10.31 (br s, 0.5H), 9.86 (br s, 0.5H), 8.80 (br s, 0.5H), 8.22 (br s, 2H), 7.85 (br s, 1H), 7.40-7.53 (m, 2H), 6.83-6.93 (m, 2H), 4.10-4.16 (m, 1H), 3.86-3.94 (m, 2H), 1.80-2.25 (m, 4H), 1.54-1.74 (m, 2H), 1.18-1.45 (m, 10H), 0.86 (t, 3H, J=6.6 Hz).

### Example 7

### General Procedure for Synthesis of Aryl-Alkoxy Ethers Under Mitsunobu Conditions

Phenol (1.2 equiv) and triphenyl phosphine (1.2 equiv) were added to an ice cold solution of the substituted phenyl alcohols (1.0 equiv) in DCM. To this mixture on ice was added DEAD or DIAD drop-wise while maintaining the temperature of the reaction mixture under 5° C. The reaction mixture was then allowed to gradually warm to room temperature and stirred overnight. The organic layer was extracted with water, 10% NH₄Cl and then brine. The combined organic layer was dried with MgSO₄ and the solvent evaporated under reduced pressure to give yellow oil which was purified by silica-gel chromatography, EtOAc-Hexane gradient. The fractions corresponding to the product were pooled and the solvent removed in vacuo to give the desired product.

### 1-Phenoxy-4-phenyl butane

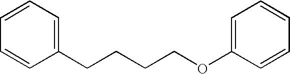

The final product was obtained as yellow oil after column chromatography, in 67% yield. ¹H NMR (400 MHz, CDCl₃) δ 7.28 (m, 4H), 7.18 (m, 3H), 6.91 (m, 3H), 3.96 (t, 2H, J=6.0 Hz), 2.68 (t, 2H, J=6.8 Hz), 1.82 (m, 4H).

### 1-Phenoxy-5-phenyl pentane

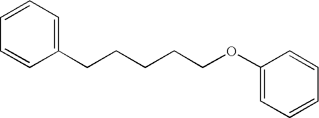

The final product was obtained as oil after column chromatography, in 37% yield. ¹H NMR (400 MHz, CDCl₃) δ 7.27 (m, 4H), 7.18 (d, 3H, J=7.2 Hz), 6.93 (dd, 1H, J=1.0 and 6.8 Hz), 6.88 (m, 2H), 3.94 (t, 2H, J=6.4 Hz), 2.64 (t, 2H, J=8.0 Hz), 1.81 (m, 2H), 1.69 (m, 2H), 1.52 (m, 2H).

### 2-Bromo-1-[4-(phenyl-butoxy)phenyl]-ethanone

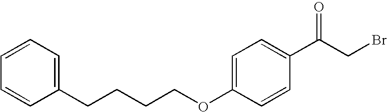

The final product was obtained as a white solid after column chromatography, in 25% yield. ¹H NMR (400 MHz, CDCl₃) δ 7.91 (d, 2H, J=8.4 Hz), 7.33-7.25 (m, 4H), 6.87-6.95 (m, 3H), 4.43 (s, 2H), 3.97 (t, 2H, J=5.6 Hz), 2.76 (t, 2H, J=7.6 Hz), 1.82 (m, 4H).

### 2-Bromo-1-[4-(5-phenyl-pentyloxy)phenyl]-ethanone

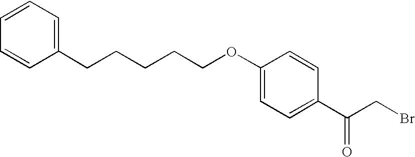

The final product was obtained as a white solid after column chromatography, in 61% yield. ¹H NMR (400 MHz, CDCl₃) δ 7.90 (d, 2H, J=8.4 Hz), 7.24-7.30 (m, 4H), 7.18 (d, 2H, J=6.4 Hz), 6.86-6.89 (m, 2H), 4.43 (s, 2H), 3.94 (t, 2H, J=6.4 Hz), 2.71 (t, 1H, J=7.6 Hz), 2.64 (t, 1H, J=7.6 Hz), 1.81 (m, 2H), 1.69 (m, 2H), 1.51 (m, 2H).

### (R)-(2-Hydroxy-1-methyl-1-{5-[4-(4-phenyl-butoxy)-phenyl]-1H-imiazol-2-yl}ethyl)-carbamic acid tert-butyl ester

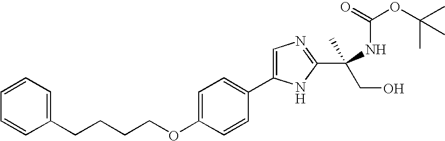

The final product was obtained as yellow oil after column chromatography, in 63% yield. ¹H NMR (400 MHz, CDCl₃) δ 7.22-7.26 (m, 4H), 7.21 (m, 2H), 7.17 (s, 1H), 6.87-6.94 (m, 2H), 4.33 (d, 1H, J=11.6 Hz), 3.97 (t, 2H, J=5.6 Hz), 3.35 (d, 1H, J=12.0 Hz), 2.69 (t, 2H, J=7.2 Hz), 2.53 (s, 2H), 1.82 (m, 4H), 1.67 (s, 3H), 1.44 (s, 9H).

### (R)-(2-Hydroxy-1-methyl-1-{5-[4-(5-phenyl-pentyloxy)-phenyl]-1H-imiazol-2-yl}ethyl)-carbamic acid tert-butyl ester

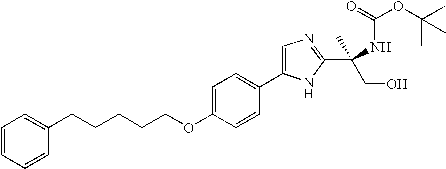

The final product was obtained as yellow oil after column chromatography, in 63% yield. ¹H NMR (400 MHz, CDCl₃) δ 7.56 (d, 2H, J=7.2Hz), 7.24-7.28 (m, 2H), 7.17 (d, 2H, J=8.0Hz), 7.13 (s, 1H), 6.86-6.89 (m, 2H), 5.77 (s, 1H), 4.27 (d, 1H, J=11.2 Hz), 3.94 (t, 2H, J=6.4 Hz), 3.64 (d, 1H, J=11.6 Hz), 2.63 (t, 2H, J=7.6 Hz), 1.81 (m, 2H), 1.69 (m, 2H), 1.66 (s, 3H), 1.42 (s, 9H), 1.26 (m, 2H).

### (R)-2-Amino-2-{5-[4-(4-phenyl-butoxy)-phenyl]-1H-imiazol-2-yl}-propan-1-ol

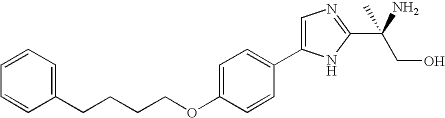

The compound was obtained as a white solid after HPLC purification. Yield: 50%, (60 mg). MS (ESI, M+H⁺)=366.3

### (R)-2-Amino-2-{5-[4-(5-phenyl-pentyloxy)-phenyl] -1H-imiazol-2-yl}-propan-1-ol

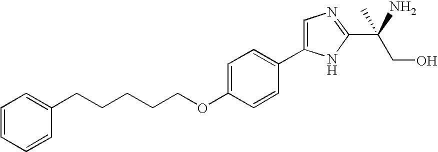

The compound was obtained as a white solid after HPLC purification. Yield: 49%, (60 mg). MS (ESI, M+H⁺)=380.3

### (R)-2-Amino-2-(5-(4-(biphenylethyloxy)phenyl)-1H-imidazol-2-yl)propan-1-ol

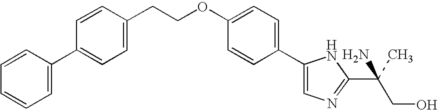

MS (ESI, M+H⁺)=414; ¹H NMR (400 MHz, DMSO-d₆) δ 8.4 (bs, 2H), 7.7 (m, 4H), 7.5 (m, 4H), 7.3 (d, 3H), 6.9 (d, 2H), 5.7 (bs), 4.18 (t, 2H), 3.7 (d, 1H), 3.6 (d, 1H), 3.04 (t, 2H), 1.45 (s, 3H).

### (R)-2-Amino-2-{4-[4-(4-propoxy-phenoxy)-phenyl]-1H-imiazol-2-yl}-propan-1-ol

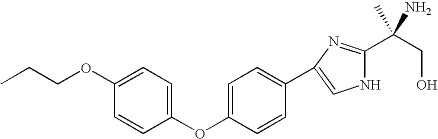

The compound was obtained as a white solid after HPLC purification. Yield: 60%, (10 mg). MS (ESI, M+H⁺)=367.5

### (R)-Phosphoric acid mono-(2-amino-2-{5-[4-(4-phenyl-butoxy)-phenyl]-1H-imidazol-2-yl}-propyl) ester

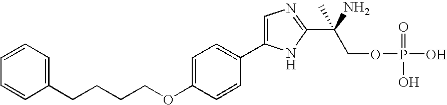

The compound was obtained as a white solid after HPLC purification. Yield: 32%, (25mg). MS (ESI, M+H⁺)=446.4

### (R)-Phosphoric acid mono-(2-amino-2-{5-[4-(4-phenyl-pentyloxy)-phenyl]-1H-imidazol-2-yl}-propyl) ester

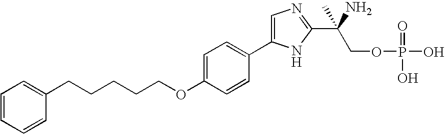

The compound was obtained as a white solid after HPLC purification. Yield: 39%, (41mg). MS (ESI, M+H⁺)=459.2

### (R)-2-Amino-2-(5-(4-(biphylenethyloxy)phenyl)-1H-imidazol-2-yl)propyl dihydrogen phosphate

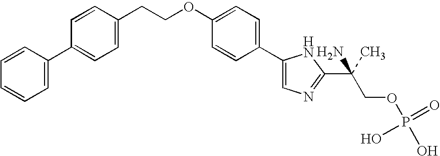

This compound was synthesized from tert-butyl (R)-1-hydroxy-2-(5-(4-(biphenylethyloxy)phenyl)-1H-imidazol-2-yl)propan-2-ylcarbamate (46 mg) to yield 9.2 mg solid product. MS (ESI, M+H⁺)=494; ¹H NMR (400 MHz, DMSO-d₆) δ 9.4 (s), 8.2 (s, 1H), 7.7 (m, 6H), 7.5 (m, 5H), 7.3 (m, 1H), 6.9 (d, 2H), 5.7 (br s), 4.25 (t, 2H), 4.15 (t, 2H), 4.05 (m, 1H), 3.9 (q, 1H), 3.1 (t, 2H), 1.45 (s, 3H).

### Example 8

### Synthesis in Biphenyl Amide Series

Several biphenyls were synthesized using the process described in Scheme 7. Microwave assisted Suzuki cross-coupling of substituted aryl boronic acids with substituted anilines afforded good to excellent yields of the biaryl amine intermediates. Furthermore, the acylation of the substituted biaryl amines with the desired headpiece followed by deprotection of the Boc group afforded the final compounds. 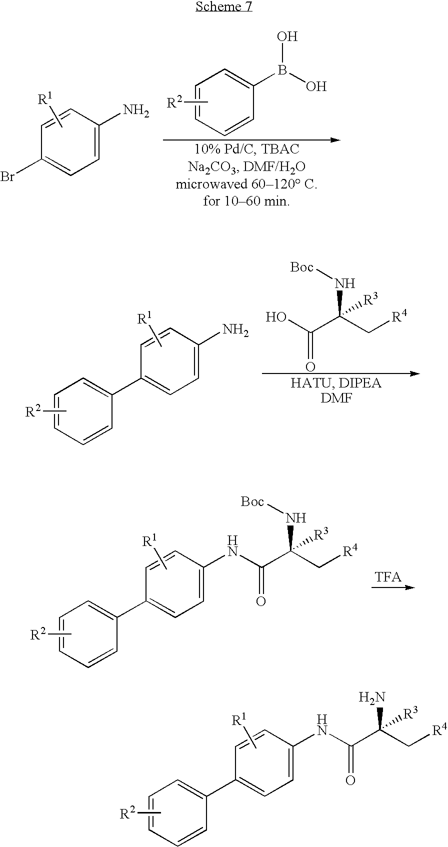
General Procedure for Suzuki Cross-Coupling:

To a mixture of a substituted bromoaniline (1.0 equiv), substituted aryl boronic acid (1.2 equiv), 10% Pd on carbon (0.1 equiv), tetrabutylammonium chloride (0.1 equiv), and sodium carbonate (1.0 equiv), in a microwave tube was added a 1:1 mixture of DMF:H₂O. The mixture was then heated to 70° C. for 20-60 minutes using a microwave. The reaction was then diluted with EtOAc (25 mL) and washed with H₂O (2×25 mL) then the solvent removed in vacuo. The crude product was purified by silica gel column chromatography using Combi-Flash system (Hex:EtOAc) as needed.

### (S)-2-Amino-N-(4-(3-isopropylphenyl)phenyl)-3-hydroxy-2-methylpropanamide

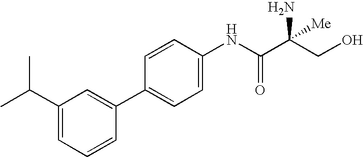

MS (ESI, M+H⁺)=313.6; ¹H NMR (400 MHz, DMSO-d₆) δ 10.96 (br s, 1H), 8.18 (br s, 2H), 7.63-7.74 (m, 4H), 7.41-7.51 (m, 2H), 7.35 (t, 1H, J=7.6 Hz), 7.21 (d, 1H, J=7.6 Hz), 5.79 (t, 1H, J=4.8 Hz), 4.00 (dd, 1H, J=11.6 Hz, J=4.8 Hz), 3.65 (dd, 1H, J=11.6 Hz, J=5.2 Hz), 2.86-3.02 (m, 1H), 1.50 (s, 3H), 1.24 (d, 6H, J=7.6 Hz).

### (S)-2-Amino-N-(4-(3-methoxyphenyl)phenyl)-3-hydroxy-2-methylpropanamide

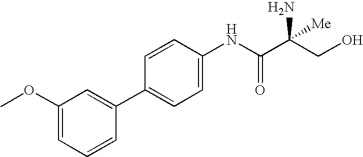

MS (ESI, M+H⁺)=301.7; ¹H NMR (400 MHz, DMSO-d₆) δ 10.98 (br s, 1H), 8.19 (br s, 2H), 7.55-7.64 (m, 4H), 7.34 (t, 1H, J=7.6 Hz), 7.16-7.24 (m, 2H), 6.88-6.94 (m, 1H), 5.80 (br s, 1H), 4.00 (dd, 1H, J=11.6 Hz, J=4.8 Hz), 3.80 (s, 3H), 3.64 (dd, 1H, J=11.6 Hz, J=5.2 Hz), 1.50 (s, 3H).

### (S)-2-Amino-N-(4-(3-ethoxyphenyl)phenyl)-3-hydroxy-2-methylpropanamide

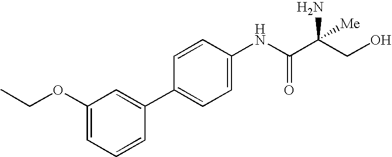

MS (ESI, M+H⁺)=315.6; ¹H NMR (400 MHz, DMSO-d₆) δ 9.97 (br s, 1H), 8.18 (br s, 2H), 7.64-7.73 (m, 4H), 7.33 (t, 1H, J=7.6 Hz), 7.14-7.23 (m, 2H), 6.86-6.91 (m, 1H), 5.80 (br s, 1H), 4.08 (q, 2H, J=7.2 Hz), 4.00 (dd, 1H, J=11.6 Hz, J=4.8 Hz), 3.64 (dd, 1H, J=11.6 Hz, J=5.2 Hz), 1.50 (s, 3H), 1.33 (t, 3H, J=7.2 Hz).

### (S)-2-Amino-N-(4-(3-propoxyphenyl)phenyl)-3-hydroxy-2-methylpropanamide

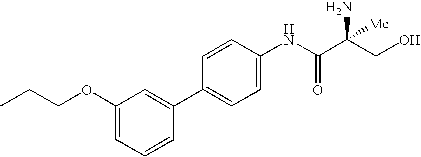

MS (ESI, M+H⁺)=329.7; ¹H NMR (400 MHz, DMSO-d₆) δ 9.96 (br s, 1H), 8.18 (br s, 2H), 7.64-7.74 (m, 4H), 7.33 (t, 1H, J=7.6 Hz), 7.13-7.22 (m, 2H), 6.86-6.92 (m, 1H), 5.80 (br t, 1H, J=4.5 Hz), 4.00 (dd, 1H, J=11.6 Hz, J=4.8 Hz), 3.98 (t, 2H, J=7.2 Hz), 3.65 (dd, 1H, J=11.6 Hz, J=5.2 Hz), 1.68-1.80 (m, 2H), 1.50 (s, 3H), 1.00 (t, 3H, J=7.2 Hz).

### (S)-2-Amino-N-(4-(3-isopropoxyphenyl)phenyl)-3-hydroxy-2-methylpropanamide

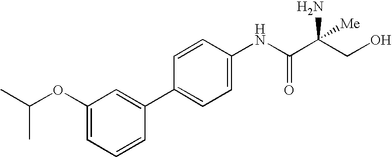

MS (ESI, M+H⁺)=329.8; ¹H NMR (400 MHz, DMSO-d₆) δ 9.96 (br s, 1H), 8.18 (br s, 2H), 7.62-7.73 (m, 4H), 7.32 (t, 1H, J=7.6 Hz), 7.11-7.20 (m, 2H), 6.86-6.92 (m, 1H), 5.80 (br t, 1H, J=4.5 Hz), 4.55-4.80 (m, 1H), 4.00 (dd, 1H, J=11.6 Hz, J=4.8 Hz), 3.65 (dd, 1H, J=11.6 Hz, J=5.2 Hz), 1.50 (s, 3H), 1.28 (d, 6H, J=7.2 Hz).

### (S)-2-Amino-N-(4-(3-n-butoxyphenyl)phenyl)-3-hydroxy-2-methylpropanamide

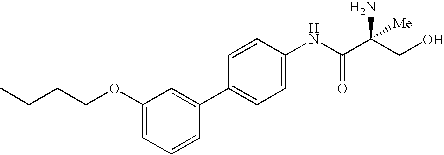

MS (ESI, M+H⁺)=343.5; ¹H NMR (400 MHz, DMSO-d₆) δ 9.97 (br s, 1 H), 8.18 (br s, 2H), 7.64-7.74 (m, 4H), 7.33 (t, 1H, J=7.6 Hz), 7.13-7.22 (m, 2H), 6.86-6.92 (m, 1H), 5.79 (br t, 1H, J=4.5 Hz), 4.03 (t, 2H, J=7.2 Hz), 4.00 (dd, 1H, J=11.6 Hz, J=4.8 Hz), 3.64 (dd, 1H, J=11.6 Hz, J=5.2 Hz), 1.65-1.75 (m, 2H), 1.50 (s, 3H), 1.49-1.52 (m, 2H), 0.92 (t, 3H, J=7.2 Hz).

### (S)-2-Amino-N-(4-(3-benzyloxyphenyl)phenyl)-3-hydroxy-2-methylpropanamide

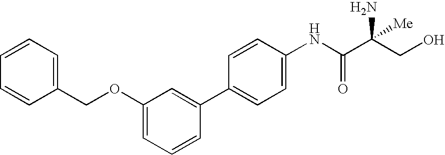

MS (ESI, M+H⁺)=377.5; ¹H NMR (400 MHz, DMSO-d₆) δ 9.96 (br s, 1H), 8.18 (br s, 2H), 7.64-7.74 (m, 4H), 7.44-7.82 (m, 2H), 7.29-7.42 (m, 6H), 6.96-7.00 (m, 1H), 5.79 (br t, 1H, J=4.5 Hz), 5.17 (s, 2H), 4.00 (dd, 1H, J=11.6 Hz, J=4.8 Hz), 3.64 (dd, 1H, J=11.6 Hz, J=5.2 Hz), 1.50 (s, 3H).

### Example 9

### General Procedure for Synthesis of Substituted Biaryl Ether/Thioether Analogs

The 4-iodophenyl-4-nitrophenoxy ethers were synthesized by reacting 4-iodophenol with 4-fluoro-nitrobenzene in the presence of a base K^{t}OBu in THF at 50° C. (Scheme 2). The nitro group was reduced using SnCl₂ in EtOH at 70° C., followed Suzuki cross-coupling then acylation of the amine with L-(Boc)-α-Me-Ser-OH using HATU. The Boc-group can then be removed using TFA in DCM or the protected material is used to synthesize the phosphate before deprotection.

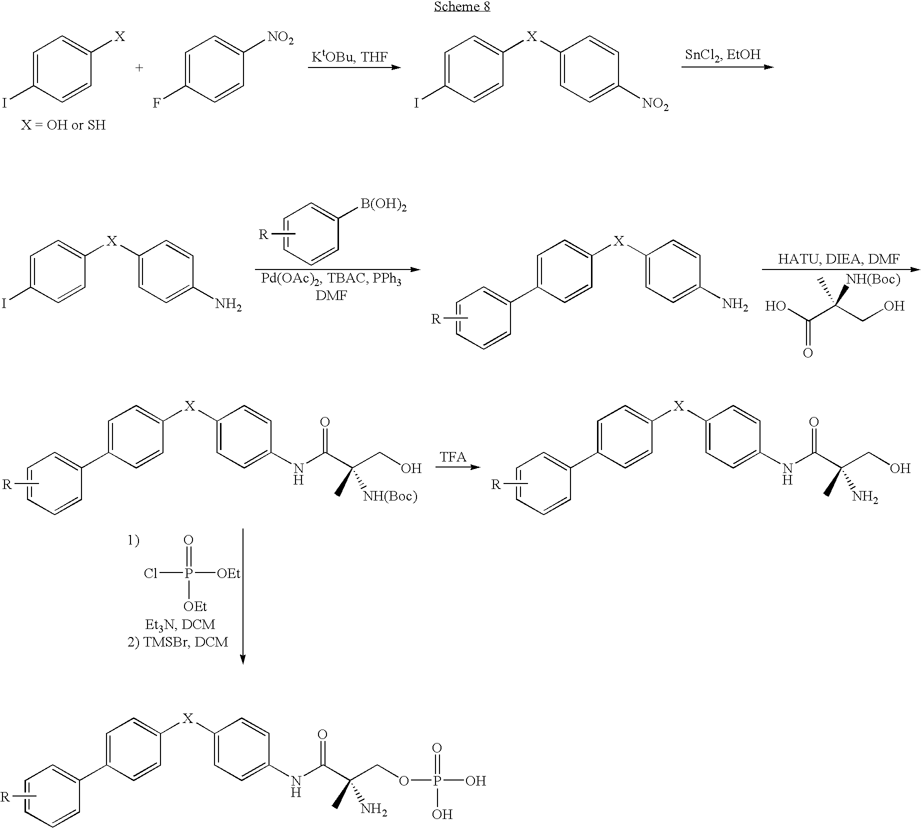

### General procedure for the synthesis of 4-(4iodophenoxy)-nitrobenzene

To a THF solution of 4-iodophenol (1.0 g, 1.0 equiv) is added K^{t}OBu (1.0M in THF, 1.0 equiv). The solution is stirred at room temperature for approximately 5 minutes and then a solution of 4-fluoro-nitrobenzene (1.1 equiv) is added dropwise. The reaction mixture is then heated to 50° C. using an oil bath and the reaction progress monitored by TLC (EtOAc:Hexane, 0.5:9.5). The reaction is complete when no more 4-iodophenol is detected by TLC. The reaction is then cooled to room temperature and put in an ice bath. Water is added slowly to quench the unreacted base, followed by extraction of the product into EtOAc. The organic layer is then washed with 10% NH4Cl and brine, dried over MgSO4, and then solvent removed under reduced pressure. The crude product is purified using Combi-Flash silica gel column chromatography, using a Hexane/EtOAc gradient. The fractions corresponding to the product are pooled and the solvent removed in vacuo to give a yellow solid (Scheme 2).

### General procedure for synthesis of substituted 4-biayloxy aniline

To a DMF solution of the 4-(haloaryloxy)-aniline (1.0 equiv) and substituted aryl boronic acid in a microwave tube, was added Pd(OAc)₂ (0.1 equiv), triphenyl phosphine (0.2 equiv), cesium carbonate (1.0-2.0 equiv) and TBAC (0.1 equiv). The reaction was then sealed and heated at 70° C. for 3-18 hours using an oil bath. The reaction mixture was filtered through a bed of Celite and then diluted with EtOAc (25 mL), washed with water (2×10 mL) and then brine (1×10 mL). The organic layer was then dried over MgSO₄, and then was solvent removed under reduced pressure. The crude product was purified using Combi-Flash silica gel column chromatography, using a Hexane/EtOAc gradient.

### 4-(4-Iodophenoxy)-nitrobenzene

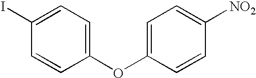

The final product was obtained as a yellow solid after purification in 73% yield. ¹H NMR (400 MHz, CDCl₃) δ 8.21(d, 2H, J=8.6 Hz), 7.73(d, 2H, J=8.8 Hz), 7.02 (d, 2H, J=9.2 Hz), 6.86 (d, 2H, J=8.8 Hz).

### 4-(4-Iodophenoxy)-phenylamine

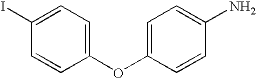

The final product was obtained as a brown solid after purification in 45% yield. ¹H NMR (400 MHz, CDCl₃) δ 7.54 (d, 2H, J=8.8 Hz), 6.84 (d, 2H, J=8.4 Hz), 6.66-6.70 (m, 4H).

### 4-(4′-Methoxy-biphenyl-4-yloxy)-phenylamine

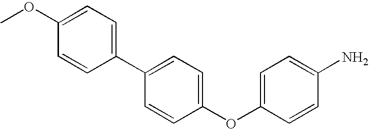

The final product was obtained as an off white solid after purification in 95% yield. ¹H NMR (400 MHz, CDCl₃) δ 7.44-7.48 (m, 4H), 6.94-6.98 (m, 4H), 6.90 (d, 2H, J=8.4 Hz), 6.69 (d, 2H, J=8.8 Hz), 3.84 (s, 3H).

### 4-(4′-Chloro-biphenyl-4-yloxy)-phenylamine

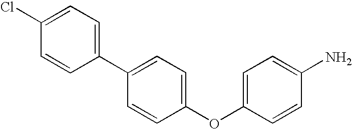

The final product was obtained as an off white solid after purification in 90% yield. ¹H NMR (400 MHz, CDCl₃) δ 7.44-7.47 (m, 4H), 7.37 (d, 2H, J=6.4 Hz), 6.97 (d, 2H, J=8.8 Hz), 6.90 (d, 2H, J=8.8 Hz), 6.70 (d, 2H, J=8.8 Hz).

### 4-(4′-tert-Butyl-biphenyl-4-yloxy)-phenylamine

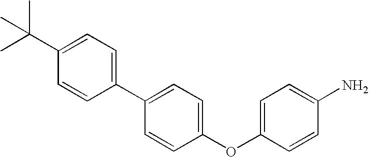

The final product was obtained as an off white solid after purification in 60% yield. ¹H NMR (400 MHz, CDCl₃) δ 7.43-7.50 (m, 6H), 6.98 (d, 2H, J=8.8 Hz), 6.91 (d, 2H, J=8.0 Hz), 6.70 (d, 2H, J=8.0 Hz), 1.37 (s, 9H).

### 4-([1,1′,4′,1″]Terphenyl-4-yloxy)-phenylamine

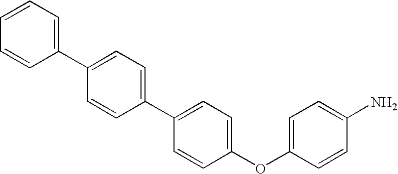

The final product was obtained as an off white solid after purification in 40% yield. ¹H NMR (400 MHz, CDCl₃) δ 7.81 (d, 1H, J=7.2 Hz), 7.77 (d, 2H, J=8.0 Hz), 7.70 (d, 2H, J=7.2 Hz), 7.63-7.64 (m, 4H), 7.55 (d, 2H, J=8.4 Hz), 7.01 (d, 2H, J=8.8 Hz), 6.92 (d, 2H, J=8.8 Hz), 6.71 (d, 2H, J=8.8 Hz).

### (S)-{2-Hydroxy-1-[4-(4′-methoxy-biphenyl-4-yloxy)-phenylcarbamoyl]-1-methyl-ethyl}carbamic acid tert-butyl ester

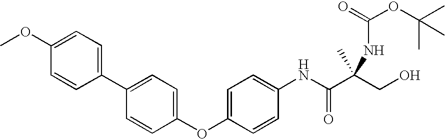

The final product was obtained as a white solid after HPLC, in 94% yield. ¹H NMR (400 MHz, CDCl₃) δ 7.47-7.50 (m, 6H,), 7.01-7.04 (m, 4H), 6.96 (d, 2H, J=8..8 Hz), 4.03 (br. s, 1H), 3.85 (s, 3H) 3.57 (d, 1H, J=11.2 Hz), 1.59 (s, 3H), 1.47 (s, 9H).

### (S)-(1-[4-(4′-Chloro-biphenyl-4-yloxy)-phenylcarbamoyl]-2-hydroxy-1-methyl-ethyl)carbamic acid tert-butyl ester

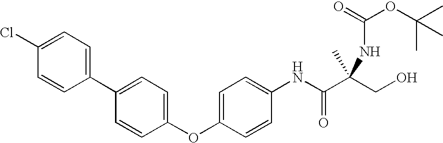

The final product was obtained as an off white solid after purification in 40% yield. ¹H NMR (400 MHz, CDCl₃) δ 7.43-7.52 (m, 6H), 6.97 (d, 2H, J=8.8 Hz), 6.90 (d, 2H, J=8.8 Hz), 6.70 (d, 2H, J=8.8 Hz) 4.03 (br. s, 1H), 3.57 (br.s, 1H,), 1.56 (s, 3H), 1.44 (s, 9H).

### (S)-{1-[4-(4′-tert-Butyl-biphenyl-4-yloxy)-phenylcarbamoyl]-2-hydroxy-1-methyl-ethyl)carbamic acid tert-butyl ester

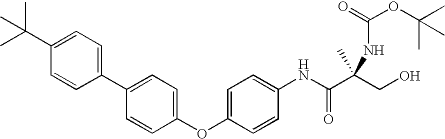

The final product was obtained as an off white solid after purification in 65% yield. ¹H NMR (400 MHz, CDCl₃) δ 7.49-7.53 (m, 6H), 7.44-7.46 (m, 2H), 7.026 (dd, 4H, J=2.4 and 8.8 Hz) 4.08 (br. s, 1H), 3.62 (br.s, 1H,), 1.59 (s, 3H), 1.47 (s, 9H), 1.36 (s, 9H).

### (S)-{2-Hydroxy-1-methyl-1-[4-(1,1′,4′,1″]terphenyl-4-yloxy)-phenylcarbamoyl]-ethyl}carbamic acid tert-butyl ester

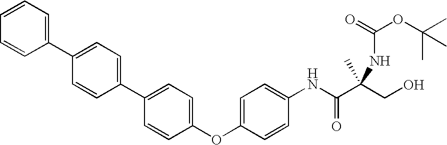

The final product was obtained as an off white solid after purification in 25% yield. ¹ H NMR (400 MHz, CDCl₃) δ 7.63-7.68 (m, 6H), 7.59 (d, 2H, J=8.8 Hz), 7.52 (d, 2H, J=8.8 Hz), 7.45 (t, 2H, J=7.6 Hz), 7.36 (m, 1H), 7.05 (dd, 4H, J=2.4 and 8.8 Hz), 3.62 (br. s, 1H), 3.40 (br.s, 1H,), 1.60 (s, 3H), 1.47 (s, 9H), 1.47 (s, 9H).

### (S)-2-Amino-N-[4-(benzo[1,3]dioxol-5-yloxy)-phenyl)-3-hydroxy-2-methyl-propionamide

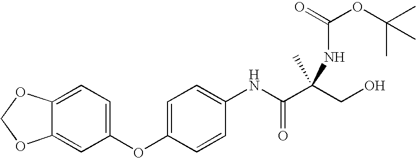

The final product was obtained as a white solid after HPLC, in 35% yield. ¹H NMR (400 MHz, CDC₁₃) δ 7.37 (d, 2H, J=8.8 Hz), 6.86 (d, 2H, J=8.8 Hz), 6.66 (d, 1H, J=8.4 Hz), 6.48 (d, 1H, J=2.4 Hz), 6.38 (dd, 1H, J=2.4 and 8.4 Hz), 5.89 (s, 2H), 4.13 (s, 1H), 3.51 (s, 1H), 1.519 (s, 3H) 1.398 (s, 9H). MS (ESI, M+H⁺)=331.1

### (S)-2-Amino-N-[4-(biphenyl-4-yloxy)-phenyl]3-hydroxy-2-methyl-propionamide

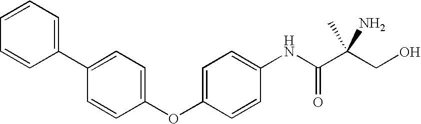

The compound was obtained as a white solid after HPLC purification. Yield: 30%, (33 mg). MS (ESI, M+H⁺)=362.2

### (S)-2-Amino-3-hydroxy-N-[4-(4′-methoxy-biphenyl-4-yloxy)-phenyl]-2-methyl-propionamide:

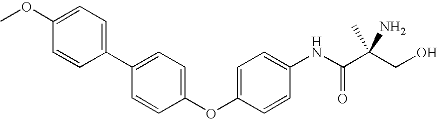

The compound was obtained as a white solid after HPLC purification. Yield: 90%, (25 mg). MS (ESI, M+H⁺)=393.7

### (S)-2-Amino-3-hydroxy-N-[4-(4′-chloro-biphenyl-4-yloxy)-phenyl]-2-methyl-propionamide

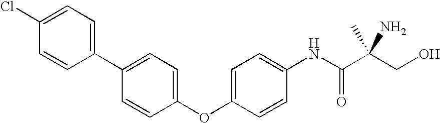

The compound was obtained as a white solid after HPLC purification. Yield: 80%, (23 mg). MS (ESI, M+H⁺)=396.1

### (S)-2-Amino-N-[4-(benzo[1,3]dioxol-5-yloxy)phenyl]-3-hydroxy-2-methyl-propionamide

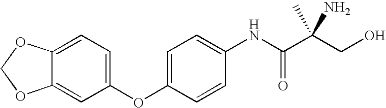

The compound was obtained as a white solid after HPLC purification. Yield: 10%, (10 mg). MS (ESI, M+H⁺)=331.7

### (S)—N-(4-(9H-Carbazol-2-yloxy)phenyl)-2-amino-3-hydroxy-2-methylpropanamide

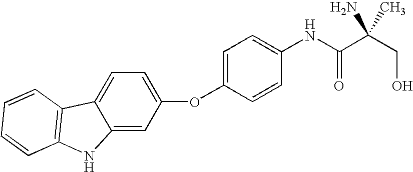

The title compound was synthesized from 2-(4-nitrophenoxy)-9H-carbazole. MS (ESI, M+H⁺)=376; ¹H NMR (400 MHz, DMSO-d₆) δ 9.92 (s, 1H), 8.17(br s, 1H), 8.07 (br s, 1H), 7.64 (br s, 2H), 7.44 (s, 1H), 7.34 (s, 1H), 7.2-7.1 (m, 2H), 7.0 (s, 1H), 6.9 (s, 1H), 5.79 (s, 1H), 3.99 (m, 1H), 3.64 (m, 1H), 1.50 (s, 3H).

### (S)—N-(4-(4-Carbonitrilephenylphenoxy)phenyl)-2-amino-3-hydroxy-2-methylpropanamide

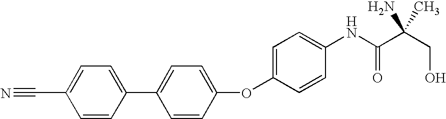

The title compound was synthesized from 4-(4-hydroxyphenyl)phenylcarbonitrile. MS (ESI, M+H⁺)=388; ¹H NMR (400 MHz, DMSO-d₆) δ 9.95 (s, 1H), 8.18 (br s, 2H), 7.92 (d, 2H), 7.86 (d, 2H), 7.75 (d, 2H), 7.67 (d, 2H), 7.12 (m, 4H), 5.79 (s, 1H), 3.99 (d, 1H), 3.64 (d, 1H), 1.50 (s, 3H).

### (S)-Phosphoric acid mono-{2-amino-2-[4-(biphenyl-4-yloxy)-phenylcarbamoyl]-propyl}ester

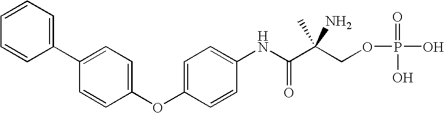

The compound was obtained as a white solid after HPLC purification. Yield: 15%, (2.5 mg). MS (ESI, M+H⁺)=443.4

### (S)-Phosphoric acid mono-{2-amino-2-14-(4′-methoxy-biphenyl-4-yloxy)-phenylcarbamoyl]-propyl}ester

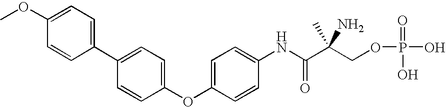

The compound was obtained as a white solid after HPLC purification. Yield: 30%, (10.0 mg). MS (ESI, M+H⁺)=443.4

### (S)-Phosphoric acid mono-{2-amino-2-[4-(4′-chloro-biphenyl-4-yloxy)-phenylcarbamoyl]-propyl}ester

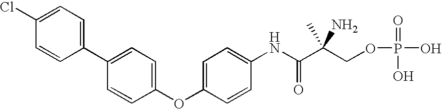

The compound was obtained as a white solid after HPLC purification. Yield: 50%, (60.0 mg). MS (ESI, M+H⁺)=477.4

### (S)-Phosphoric acid mono-{2-amino-2-[4-(4′-tert-butyl-biphenyl-4-yloxy)-phenylcarbamoyl]-propyl}ester

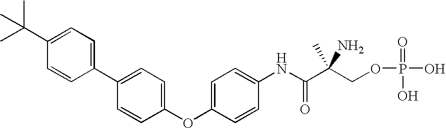

The compound was obtained as a white solid after HPLC purification. Yield: 40%, (38 mg). MS (ESI, M+H⁺)=477.4

### (S)-Phosphoric acid mono-12-amino-2-[4-(1,1′,4′1-terphenyl-4-yloxy)-phenylcarbamoyl]-propyl}ester

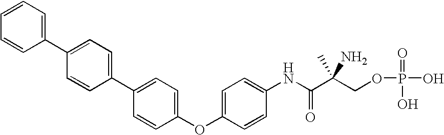

The compound was obtained as a white solid after HPLC purification. Yield: 35%, (7 mg). MS (ESI, M+H⁺)=519.2

### (S)-2-(4-(2-Phenylnaphthalen-6-yloxy)phenylcarbamoyl)-2-aminopropyl dihydrogen phosphate

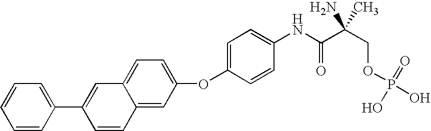

¹H NMR (400 MHz, DMSO-d₆) δ 10.15 (s, 1H), 9.4 (bs, 2H), 8.2 (d, 1H), 8.0 (d, 1H), 7.92 (m, 2H), 7.8 (m, 2H), 7.7 (m, 2H), 7.5 (m, 2H), 7.35 (m, 2H), 7.15 (m, 2H), 4.3 (t, 1H), 4.0 (t, 1H), 1.50 (s, 3H).

### 4-(4-Bromo-phenylsulfanyl)-nitrobenzene

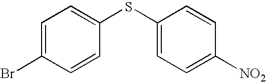

The final product was obtained as pale yellow oil after column chromatography, in 80% yield. ¹H NMR (400 MHz, CDC₁₃) δ 8.08 (d, 2H, J=9.2 Hz), 7.58 (d, 2H, J=8.8 Hz), 7.39 (d, 2H, J=8.8 Hz), 7.2 (d, 2H, J=9.2 Hz).

### 4-(4-Bromo-phenylsulfanyl)-phenylamine

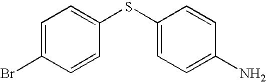

The final product was obtained as a pale yellow solid after column chromatography, in 90% yield. ¹H NMR (400 MHz, CDC₁₃) δ 7.28-7.32 (m, 4H), 6.96 (d, 2H, J=8.8 Hz), 6.68 (d, 2H, J=8.4 Hz).

### 4-(Biphenyl-4-ylsulfanyl)-phenylamine

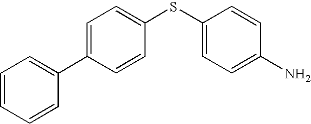

The final product was obtained as a pale yellow solid after column chromatography, in 73% yield. ¹H NMR (400 MHz, CDC₁₃) δ 7.53 (d, 2H, J=8.4 Hz), 7.45 (d, 2H, J=8.4 Hz ), 7.40 (d, 2H, J=7.6 Hz ), 7.35 (d, 2H, J=8.8 Hz ), 7.31 (m, 1H), 7.19 (d, 2H, J=8.4 Hz), 6.73 (d, 2H, J=7.2 Hz).

### (S)-(1-{4-[2-(Biphenyl-4-ylsulfanyl)-phenylcarbamoyl]-2-hydroxy-1-methyl-ethyl}-carbamic acid tert-butyl ester

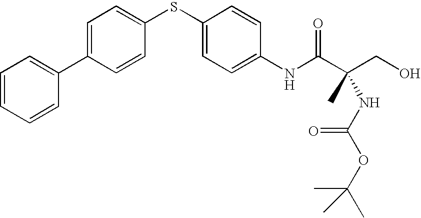

The final product was obtained as a pale yellow solid after column chromatography, in 73% yield. ¹H NMR (400 MHz, CDC₁₃) δ 7.51 (d, 2H, J=8.4 Hz), 7.44 (m, 4H), 7.39 (d, 2H, J=7.6 Hz), 7.35 (d, 2H, J=8.8 Hz), 7.31 (m, 1H), 7.19 (d, 2H, J=8.4 Hz), 4.08 (br.s, 1H), 3.67 (br. s, 1H), 1.58 (s, 3H), 1.46 (s, 9H).

### Example 10

### Synthesis of α-Methyl-Glutamate Analogs

A number of α-methyl-glutamate analogs were synthesized as potential phosphate mimics using the process described in Scheme 9. Oxidation of the alcohol in (x-methyl-serine protected precursor followed by a Wittig olefination provided conjugated methyl ester as the desired intermediate. The methyl ester intermediate was then either deprotected or hydrolyzed to provide the desired product or was taken through a hydrogenation before conversion to the desired product.

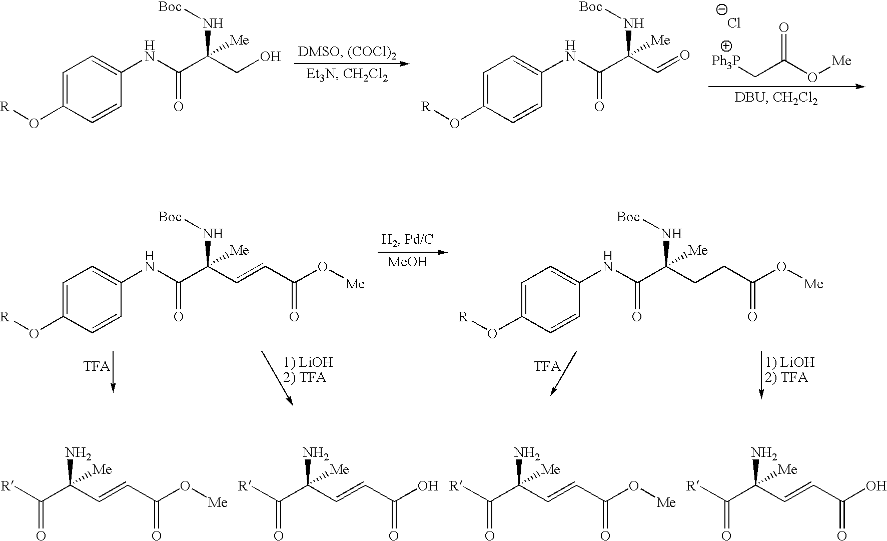

### tert-Butyl (S)-1-(4-(octyloxy)phenylcarbamoyl)-1-formylethylcarbamate

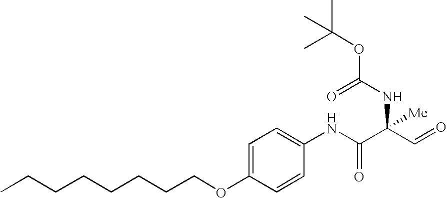

To a solution of DMSO (0.28 mL, 3.3 equiv) in dry CH₂C₁₂ (10 mL) at −78° C. was added oxalyl chloride (0.95 mL, 1.6 equiv) drop wise then stirred for 10 minutes before addition of the desired alcohol (0.50 g, 1.0 equiv) in CH₂C₁₂ (5 mL). The mixture was stirred at −78° C. for 4 hours, then triethylamine (0.83 mL, 5 equiv) was added. The reaction was allowed to warm up to room temperature and loaded directly on a silica gel column for purification using Combi-Flash system (Hex:EtOAc). The product was obtained as a yellow solid in 60% (360 mg) yield. ¹H NMR (400 MHz, CDC₁₃) δ 9.67 (s, 1H), 8.50 (br s, 1H), 7.37 (d, 2H, J=7.6 Hz), 6.85 (d, 2H, J=7.6 Hz), 5.89 (br s, 1H), 3.94 (t, 2H, J=6.8 Hz), 1.71-1.80 (m, 2H), 1.67 (s, 3H), 1.46 (s, 9H), 1.22-1.48 (m, 10H), 0.88 (t, 3H, J=6.8 Hz),.

### tert-Butyl (S,E)-2-(4-(octyloxy)phenylcarbamoyl)-4-(methoxycarbonyl)but-3-en-2-ylcarbamate

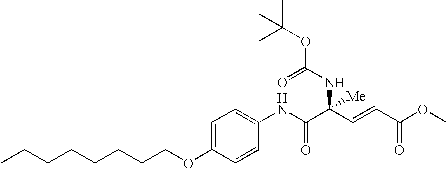

To a solution of (carbomethoxymethyl)triphenylphosphonium chloride (160 mg, 1.0 equiv) in dry CH₂C₁₂ (3 mL) at room temperature was added DBU (64 μL, 1.2 equiv) then stirred for 15 minutes before addition of the desired aldehyde (150 mg, 1.2 equiv) in CH₂C₁₂ (2 mL). The mixture was stirred at room temperature for 2 hours then directly loaded on a silica gel column for purification using Combi-Flash system (Hex:EtOAc). The product was obtained as colorless oil in 74% (125 mg) yield. ¹H NMR (400 MHz, CDC₁₃) δ 8.38 (br s, 1H), 7.37 (d, 2H, J=8.6 Hz), 6.85 (d, 2H, J=8.6 Hz), 6.04 (d, 1H, J=16.0 Hz), 5.30 (br s, 1H), 3.92 (t, 2H, J=6.8 Hz), 3.78 (s, 3H), 1.70-1.82 (m, 2H), 1.64 (s, 3H), 1.43 (s, 9H), 1.23-1.48 (m, 10H), 0.89 (t, 3H, J=6.8 Hz).

### tert-Butyl (S)-2-(4-(octyloxy)phenylcarbamoyl)-4-(methoxycarbonyl)butan-2-ylcarbamate

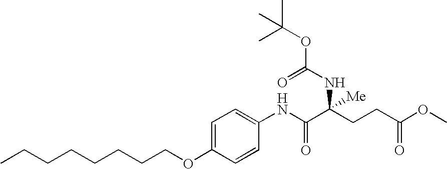

To a solution of the olefin (90 mg, 1.0 equiv) in MeOH (4 mL) was added activated Pd on carbon (9 mg in EtOAc (1 mL). The reaction was stirred under H₂ (gas) atmosphere overnight. The reaction was filtered through a layer of Celite to remove the Pd and carbon. The product was obtained as a white solid in 93% (84 mg) yield. ¹H NMR (400 MHz, CDC₁₃) δ 8.86 (br s, 1H), 7.39 (d, 2H, J=8.8 Hz), 6.85 (d, 2H, J=8.8 Hz), 5.42 (br s, 1H), 5.30 (s, 1H), 3.92 (t, 2H, J=6.8 Hz), 3.67 (s, 3H), 2.38-2.52 (m, 2H), 2.20-2.38 (m, 2H), 1.71-1.81 (m, 2H), 1.57 (s, 3H), 1.45 (s, 9H), 1.22-1.54 (m, 10H), 0.89 (t, 3H, J=6.8 Hz).

### (S,E)-Methyl 4-(4-(octyloxy)phenylcarbamoyl)-4-aminopent-2-enoate

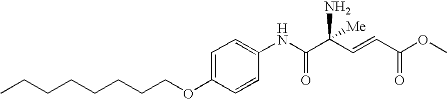

The product was obtained as colorless thick oil in 97% (14 mg) yield. MS (ESI, M+H⁺)=377.7; ¹H NMR (400 MHz, DMSO-d₆) δ 9.93 (br s, 1H), 8.70 (br s, 2H), 7.44 (d, 2H, J=8.8 Hz), 7.13 (d, 1H, J=16.0 Hz), 6.90 (d, 2H, J=8.8 Hz), 6.22 (d, 1H, J=16.0 Hz), 3.91 (t, 2H, J=6.8 Hz), 3.71 (s, 3H), 1.62-1.72 (m, 2H), 1.47 (s, 3H), 1.22-1.42 (m, 10H), 0.84 (t, 3H, J=6.8 Hz).

### (S,E)-4-(4-(Octyloxy)phenylcarbamoyl)-4-aminopent-2-enoic acid

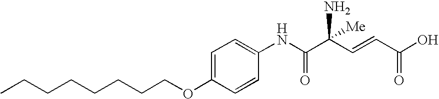

The product was obtained as a white solid in 99% (20 mg) yield. MS (ESI, M+H⁺)=363.7; ¹H NMR (400 MHz, DMSO-d₆) δ 9.95 (br s, 1H), 7.54 (d, 2H, J=8.8 Hz), 7.14 (d, 1H, J=16.0 Hz), 6.97 (d, 2H, J=8.8 Hz), 6.15 (d, 1H, J=16.0 Hz), 3.98 (t, 2H, J=6.8 Hz), 1.74 (s, 3H), 1.65-1.78 (m, 2H), 1.22-1.50 (m, 10H), 0.94 (t, 3H, J=6.8 Hz).

### (S)-Methyl 4-(4-(octyloxy)phenylcarbamoyl)-4-aminopentanoate

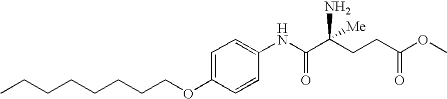

The product was obtained as colorless thick oil in 93% (13 mg) yield. MS (ESI, M+H⁺)=379.6; ¹H NMR (400 MHz, DMSO-d₆) δ 9.83 (br s, 1H), 8.26 (br s, 2H), 7.44 (d, 2H, J=8.8 Hz), 6.90 (d, 2H, J=8.8 Hz), 3.91 (t, 2H, J=6.8 Hz), 3.56 (s, 3H), 2.10-2.40 (m, 4H), 1.62-1.72 (m, 2H), 1.41 (s, 3H), 1.20-1.42 (m, 10H), 0.84 (t, 3H, J=6.8 Hz).

### (S)-4-(4-(Octyloxy)phenylcarbamoyl)-4-aminopentanoic acid

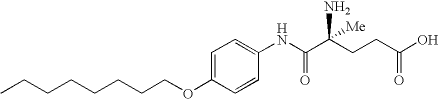

The product was obtained as a white solid in 95% (19 mg) yield. MS (ESI, M+H⁺)=365.8; ¹H NMR (400 MHz, DMSO-d₆) δ 9.88 (br s, 1H), 7.39 (d, 2H, J=8.8 Hz), 6.84 ( (d, 2H, J=8.8 Hz), 3.86 (t, 2H, J=6.8 Hz), 1.92-2.30 (m, 4H), 1.57-1.67 (m, 2H), 1.49 (s,3H), 1.15-1.38 (m, 10H), 0.81 (t, 3H, J=6.8 Hz).

Linker Modification:

A number of biphenyl-tail analogs with different linker lengths were synthesized using the process described in Scheme 10. Under Sonogashira conditions various alkynols were reacted with 4-bromobiphenyls followed by hydrogenation to afford biphenylalkyl alcohol intermediates. Reaction of the alcohol with substituted 4-fluoro-nitrobenzene under Williamson ether synthesis conditions followed by hydrogenation and coupling with amino acid provided the desired protected alcohol which was phosphorylated or deprotected to obtain the final product. 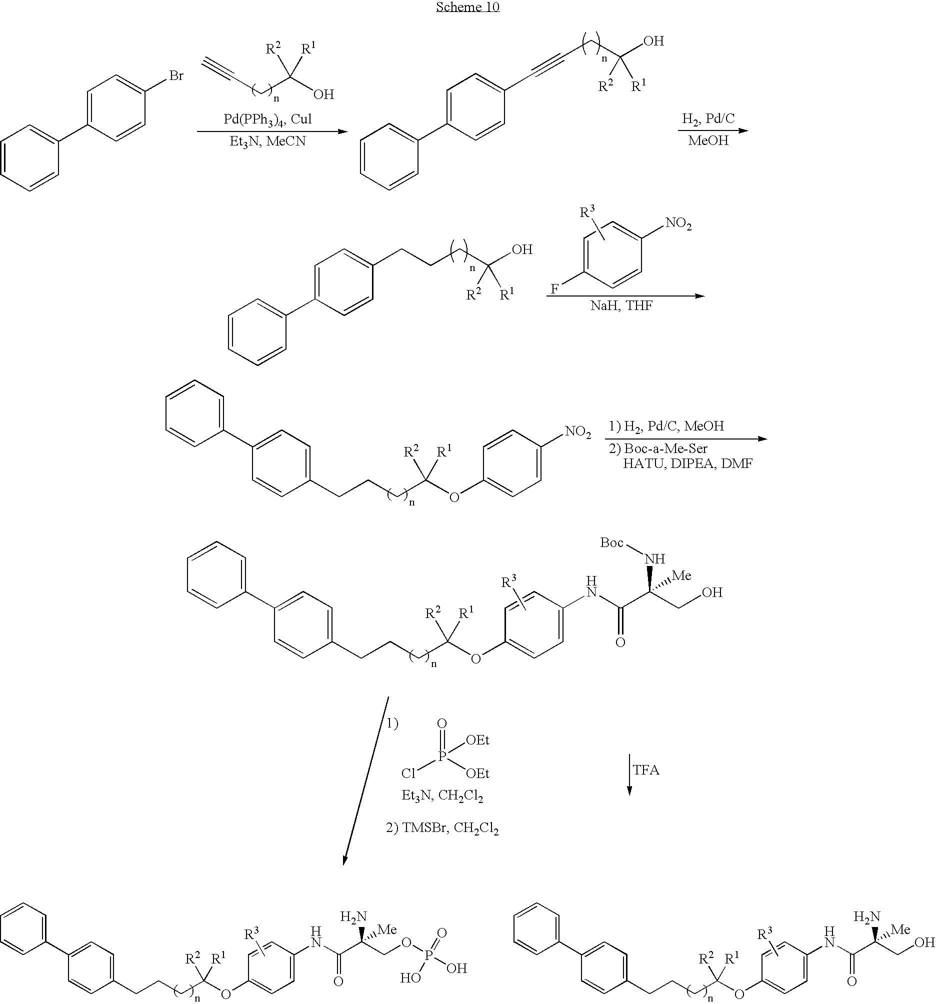
General Procedure for Sonogashira Cross-Coupling:

To a mixture of a 4-bromobiphenyl (1.0 equiv), Pd(PPh₃)₄ (0.02 equiv) and CuI (0.04 equiv) in MeCN was added the alkynol (1.5 equiv) and Et₃N (1.5 equiv). The reaction mixture was stirred for 2-16 hours at reflux, then the solvent removed in vacuo. The crude product was purified by silica gel column chromatography using the Combi-Flash system (Hex:EtOAc) as needed.

General Procedure for Williamson Ether Ssynthesis:

To a solution of biphenylalkyl alcohol (1.0 equiv) in dry THF under nitrogen 15 atmosphere was added NaH (2.5 equiv) in portions. The reaction mixture was heated at 60° C. for 15 minutes, then 4-flouro-nitrobenzene (1.0 equiv) was added and the solution stirred for 1-6 hours. The reaction was allowed to cool to room temperature then quenched with water. The mixture was then diluted with EtOAc and washed with H₂O (2×), 10% KHSO4 (1×), and saturated NaCl (1×). The product was either carried forward as is or it was purified by silica gel column chromatography using the Combi-Flash system (Hex:EtOAc).

### 3-(4-Phenylphenyl)propan-1-ol

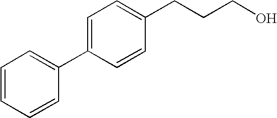

The product was obtained as a yellow solid in 57% (0.56 g) yield. ¹H NMR (400 MHz, CDC₁₃) δ 7.55-7.60 (m, 2H), 7.49-7.54 (m, 2H), 7.39-7.45 (m, 2H), 7.30-7.35 (m, 1H), 7.25-7.30 (m, 2H), 3.71 (t, 2H, J=6.8 Hz), 2.76 (t, 2H, J=6.8 Hz), 1.88-1.98 (m, 2H), 1.32 (br s, 1H).

### 4-(4-Phenylphenyl)butan-1-ol

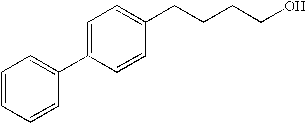

The product was obtained as a white solid in 62% (0.62 g) yield. ¹H NMR (400 MHz, CDC₁₃) δ 7.55-7.60 (m, 2H), 7.48-7.54 (m, 2H), 7.39-7.45 (m, 2H), 7.29-7.35 (m, 1H), 7.23-7.28 (m, 2H), 3.70 (t, 2H, J=6.8 Hz), 2.71 (t, 2H, J=6.8 Hz), 1.60-1.80 (m, 4H), 1.22 (br s, 1H).

### tert-Butyl (S)-2-(4-(4-(4-phenylphenyl)butan-2-yloxy)phenylcarbamoyl)-1-hydroxypropan-2-ylcarbamate:

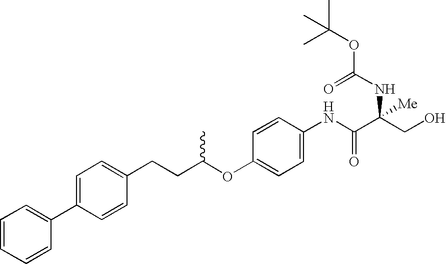

¹H NMR (400 MHz, CDC₁₃) δ 9.48 (br s, 1H), 8.11 (br s, 2H), 7.60-7.64 (m, 2H), 7.58 (d, 2H, J=8.8 Hz), 7.51 (d, 2H, J=8.6 Hz), 7.37-7.48 (m, 4H), 7.32 (t, 1H, J=8.6 Hz), 6.84 (d, 2H, J=8.8 Hz), 5.61 (br s, 1H), 4.30-4.38 (m, 1H), 4.10 (br s, 1H), 3.56 (br s, 1H), 3.28 (br s, 1H), 2.70-2.90 (m, 2H), 2.01-2.14 (m, 1H), 1.84-1.96 (m, 1H), 1.58 (s, 3H), 1.46 (s, 9H), 1.31 (d, 3H, J=7.0 Hz).

### (S)—N-(4-(3-(4-Phenylphenyl)propoxy)phenyl)-2-amino-3-hydroxy-2-methylpropanamide

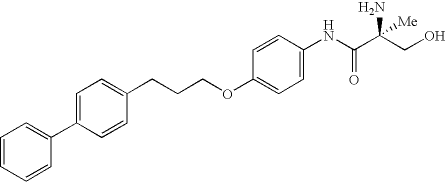

MS (ESI, M+H⁺)=405.5; ¹H NMR (400 MHz, DMSO-d₆) δ 9.74 (br s, 1H), 8.11 (br s, 2H), 7.60-7.64 (m, 2H), 7.57 (d, 2H, J=8.8 Hz), 7.47 (d, 2H, J=8.8 Hz), 7.43 (t, 2H, J=8.6 Hz), 7.28-7.35 (m, 2H), 6.92 (d, 2H, J=8.8 Hz), 3.95 (t, 2H, J=6.8 Hz), 3.93 (dd, 1H, J=12.0 Hz, J=4.8 Hz), 3.61 (dd, 1H, J=12.0 Hz, J=5.0 Hz), 2.76 (t, 2H, J=6.8 Hz), 1.98-2.08 (m, 2H), 1.47 (br s, 1H).

### (S)—N-(4-(4-(4-Phenylphenyl)butoxy)phenyl)-2-amino-3-hydroxy-2-methylpropanamide

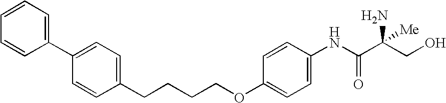

MS (ESI, M+H⁺)=419.5; ¹H NMR (400 MHz, DMSO-d₆) δ 9.73 (br s, 1H), 8.11 (br s, 2H), 7.60-7.64 (m, 2H), 7.56 (d, 2H, J=8.8 Hz), 7.40-7.49 (m, 4H), 7.27-7.35 (m, 3H), 6.91 (d, 2H, J=8.8 Hz), 3.95 (t, 2H, J=6.8 Hz), 3.94 (dd, 1H, J=12.0 Hz, J=4.8 Hz), 3.60 (dd, 1H, J=12.0 Hz, J=5.2 Hz), 2.62-2.70 (m, 2H), 1.68-1.77 (m, 4H), 1.46 (br s, 1H).

### (S)-2-(4-(3-(4-Phenylphenyl)propoxy)phenylcarbamoyl)-2-aminopropyl dihydrogen phosphate

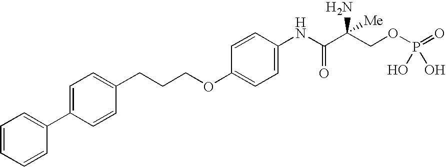

The product was obtained as a white solid in 25% (7.0 mg) yield over two steps. MS (ESI, M+H⁺)=485.6.

### (S)-2-(4-(4-(4-Phenylphenyl)butoxy)phenylcarbamoyl)-2-aminopropyl dihydrogen phosphate:

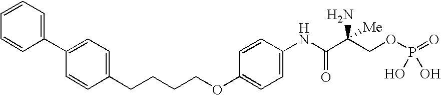

The product was obtained as a white solid in 43% (12.0 mg) yield over two steps. MS (ESI, M+H⁺)=499.6.

### (S)-2-(4-(4-(4-Phenylphenyl)butan-2-yloxy)phenylcarbamoyl)-2-aminopropyl dihydrogen phosphate

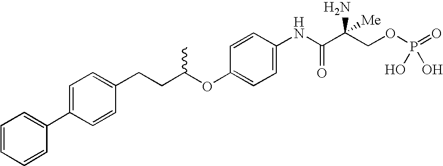

The product was obtained as a white solid in 30% (9.0 mg) yield over two steps. MS (ESI, M+H⁺)=499.6.

One Carbon Length Linker:

One carbon-ether length biphenyl-tail analogs were synthesized using the process described in Scheme 11. After N-acylation of 4-aminophenol, benzyl ether synthesis was achieved under mild alkylation condition. The biphenyl tail was synthesized using mild Suzuki cross-coupling using phenylboronic acid. The obtained protected alcohol was then further modified to phosphorylate or deprotected to produce the desired final product.

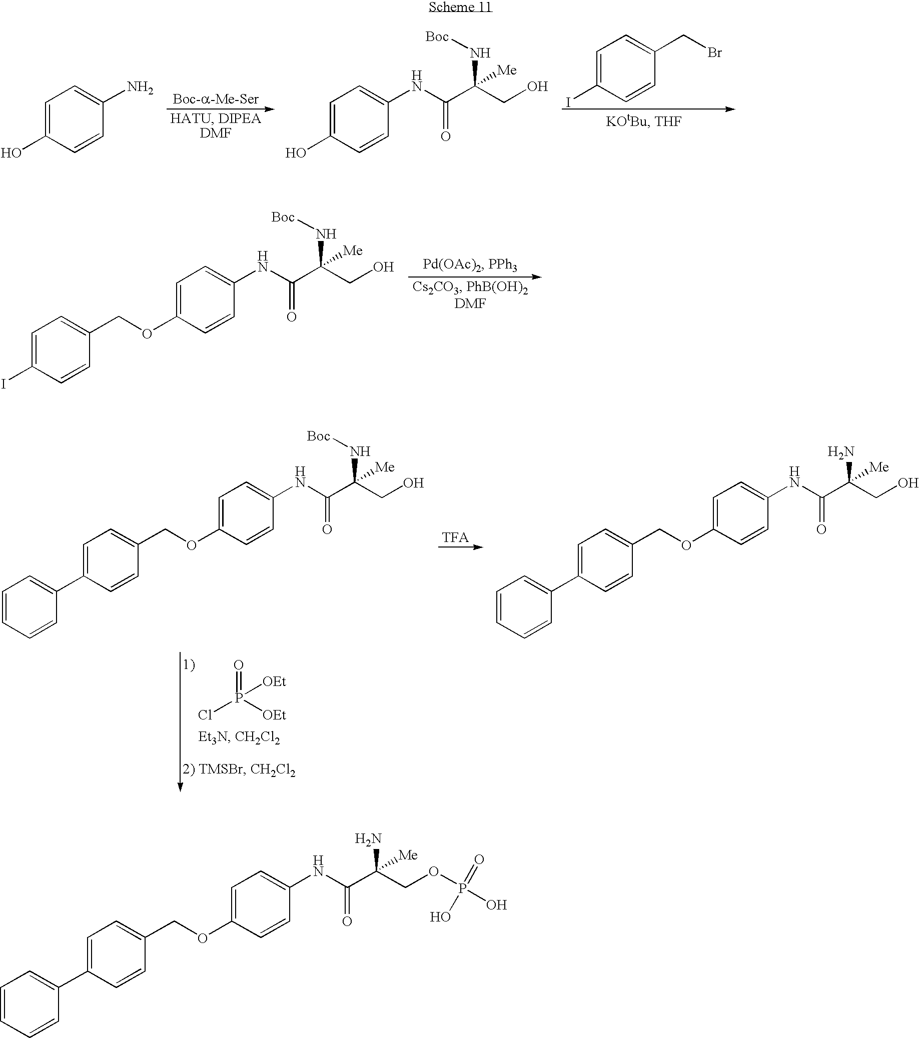

### tert-Butyl (S)-2-(4-(4-iodobenzyloxy)phenylcarbamoyl)-1-hydroxypropan-2-ylcarbamate

To a solution of N-acylated 4-aminophenol (300 mg, 1.0 equiv) in dry THF (6 mL) at room temperature was added a 1.0 M solution of KOtBu in THF (0.97 mL, 1.0 equiv) and stirred for 10 minutes before addition of 4-iodobenyl bromide (290 mg, 1.0 equiv). The solution was stirred for 3 hoursand subsequently the solvent was removed in vacuo. The crude product was purified by silica gel column chromatography using Combi-Flash system (Hex:EtOAc). The product was obtained as a white foam in 40% (203 mg) yield.

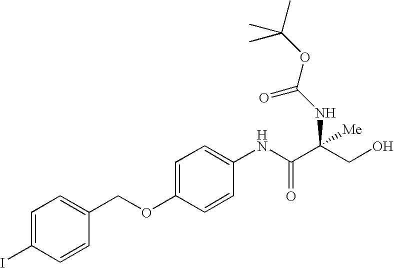

¹H NMR (400 MHz, CDC₁₃) δ 9.50 (br s, 1H), 7.70 (d, 2H, J=8.6 Hz), 7.41 (d, 2H, J=8.6 Hz), 7.18 (d, 2H, J=8.6 Hz), 6.90 (d, 2H, J=8.6 Hz), 5.60 (br s, 1H), 4.99 (s, 2H), 4.08 (br s, 1H), 3.55 (br s, 1H), 3.22 (br s, 1H), 1.58 (s, 3H), 1.46 (s, 9H).

### tert-Butyl (S)-2-(4-(4-phenylbenzyloxy)phenylcarbamoyl)-1-hydroxypropan-2-ylcarbamate

To a mixture of a substituted aryl iodide (120 mg, 1.0 equiv), phenyl boronic acid (35 mg, 1.2 equiv), Pd(OAc)₂ (5 mg, 0.1 equiv), triphenylphosphine (12 mg, 0.2 equiv), and cesium carbonate (74 mg, 1.0 equiv) was added DMF (4 mL). The mixture was heated at 50° C. for an hour. The reaction was then diluted with EtOAc (20 mL) and washed with H₂O (2×25 mL) then the solvent was removed in vacuo. The crude product was purified by silica gel column chromatography using Combi-Flash. system (Hex:EtOAc) as needed. The product was obtained as a white solid in 79% (85 mg) yield.

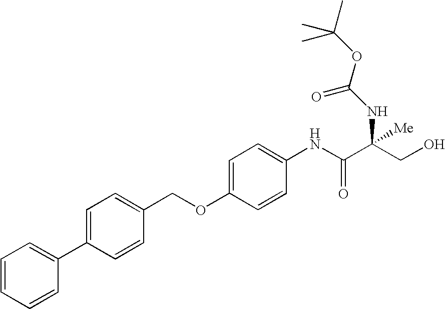

¹H NMR (400 MHz, CDC₁₃) δ 9.46 (br s, 1H), 7.57-7.63 (m, 4H), 7.40-7.52 (m, 6H), 7.32-7.38 (m, 1H), 6.98 (d, 2H, J=8.6 Hz), 5.61 (br s, 1H), 5.09 (s, 2H), 4.09 (br s, 1H), 3.56 (br s, 1H), 3.27 (br s, 1H), 1.58 (s, 3H), 1.47 (s, 9H).

### (S)—N-(4-(4-Phenylbenzyloxy)phenyl)-2-amino-3-hydroxy-2-methylpropanamide

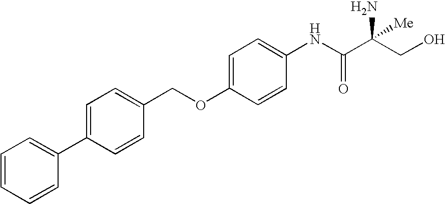

The product was obtained as a white solid in 45% (9.0 mg) yield. MS (ESI, M+H⁺)=377.4; ¹H NMR (400 MHz, DMSO-d₆) δ 9.76 (br s, 1H), 8.12 (br s, 1H), 7.63-7.69 (m, 4H), 7.42-7.53 (m, 6H), 7.32-7.38 (m, 1H), 7.02 (d, 2H, J=8.6 Hz), 5.74 (t, 1H, J=5.1 Hz), 5.13 (s, 2H), 3.94 (dd, 1H, J=11.8 Hz, J=4.7 Hz), 3.61 (dd, 1H, J=11.8 Hz, J=4.7 Hz), 1.46 (s, 3H).

### (S)-2-(4-(4-Phenylbenzyloxy)phenylcarbamoyl)-2-aminopropyl dihydrogen phosphate

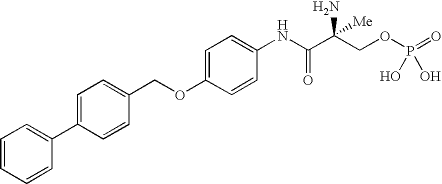

The product was obtained as a white solid in 36% (18.0 mg) yield over two steps. MS (ESI, M+H⁺)=457.1.

Thiazole Linker:

The thiazole-biphenyl analogs were synthesized using the process described in Scheme 12. Substituted benzamide was converted to thiobenzamide using Lawesson's reagent. Reaction of thioamide with bromoketone afforded the thiazole intermediate. Reduction of the nitro group followed by acylation provided an orthogonally protected intermediate, which was further modified by a mild Suzuki cross-coupling process using aryl boronic acid. The protecting Boc and the oxazolidine groups were removed using p-TsOH and the product was then phosphorylated to obtain the final phosphate product.

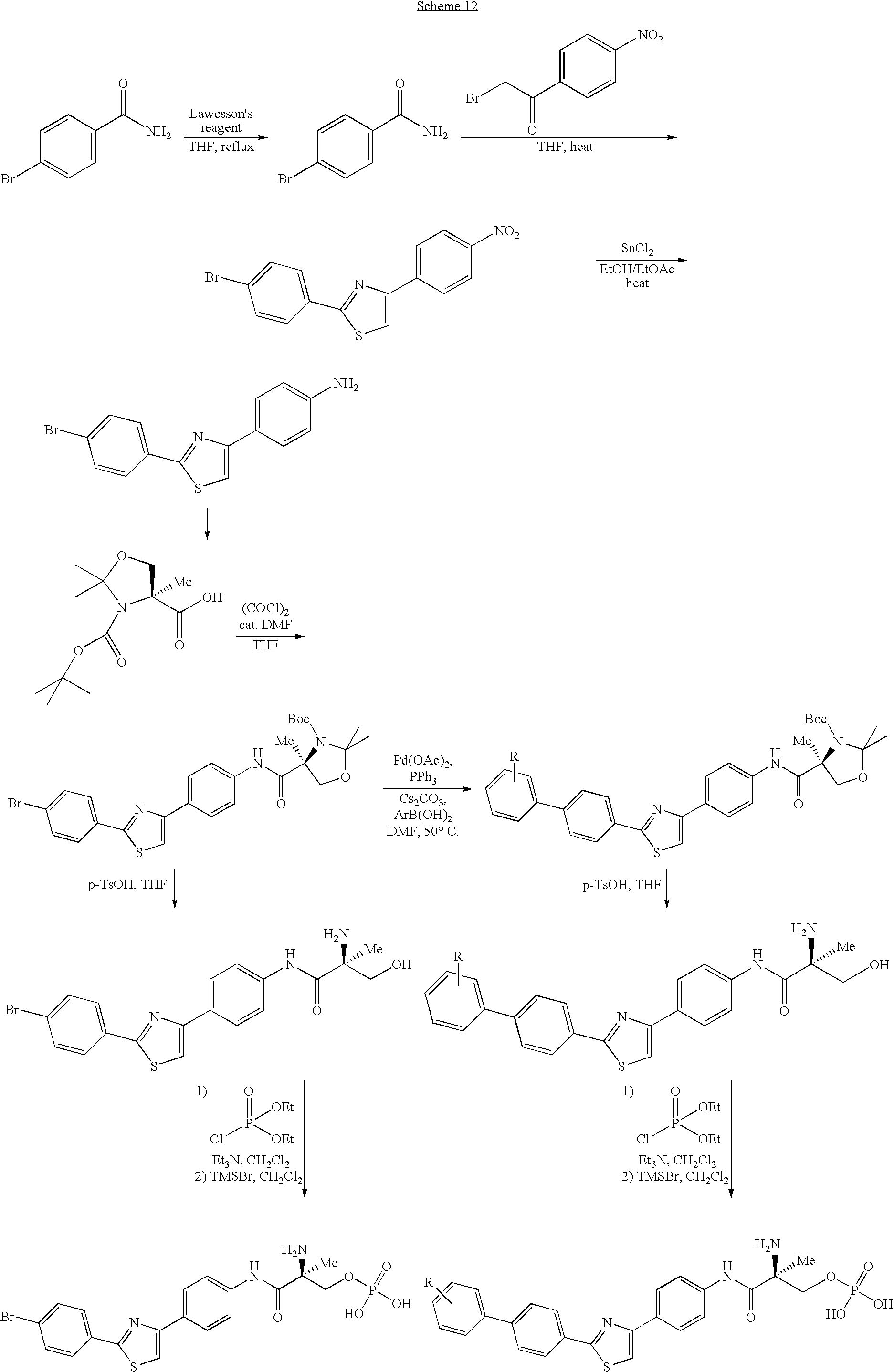

### 4-(2-(4-Bromophenyl)thiazol-4-yl)benzenamine

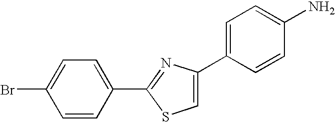

To a mixture of Lawesson's reagent (6.07 g, 1.5 equiv) and 4-bromobenzamide (2.00 g, 1.0 equiv) was added dry THF (20 mL). The reaction mixture was refluxed overnight under nitrogen atmosphere. The reaction was allowed 10 to cool to room temperature, then diluted with EtOAc (50 mL) and washed with 5% NaHCO₃ (2×50 mL) and saturated NaCl (1×25 mL). The organic layer was dried over anhydrous MgSO₄ then the solvent removed in vacuo. The crude product was purified by silica gel column chromatography using Combi-Flash system (Hex:EtOAc). The product was obtained as white solid in 99% (2.16 g) yield.

To a mixture of 4-bromothiobenzamide (2.16 g, 1.0 equiv) and 4-nitro-bromoacetophenone (2.43 g, 1.0 equiv) was added dry THF (20 mL) and heated at 60° C. for 3 hours. The solvent was removed in vacuo and the crude product was purified by silica gel column chromatography using Combi-Flash system (Hex:EtOAc). The product was obtained as yellow solid in 84% (3.00 g) yield.

To a mixture of the nitro intermediate (1.10 g, 1.0 equiv) and SnC₁₂ (3.02 g, 5.0 equiv) was added EtOH (30 mL) then heated at 80° C. for 3 hours. The reaction mixture was diluted with H₂O (50 mL) then basified to pH 10 using saturated NaOH solution. The reaction mixture was then extracted with EtOAc (2×100 mL). The organic layers were combined and removed in vacuo. The crude product was purified by silica gel column chromatography using Combi-Flash system (Hex:EtOAc). The product was obtained as yellow solid in 63% (0.63 g) yield.

¹H NMR (400 MHz, CDC₁₃) δ 7.90 (d, 2H, J=8.6 Hz), 7.79 (d, 2H, J=8.6 Hz), 7.58 (d, 2H, J=8.6 Hz), 7.26 (d, 1H, J=0.8 Hz), 6.75 (d, 2H, J=8.6 Hz), 3.80 (br s, 2H).

### (S)-tert-Butyl 4-(4-(2-(4-bromophenyl)thiazol-4-yl)phenylcarbamoyl)-2,2,4-trimethyloxazolidine-3-carboxylate

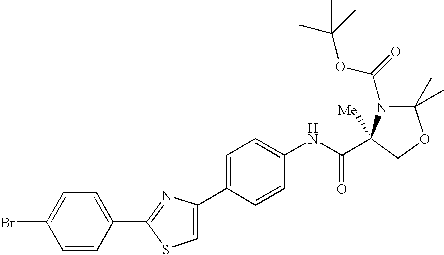

To a solution of (S)-3-(tert-butoxycarbonyl)-2,2,4-trimethyloxazolidine-4-carboxylic acid (100 mg, 1.0 equiv) in dry THF (5 mL) was added a 2.0 M solution of oxalyl chloride in CH₂C₁₂ (0.23 mL, 1.2 equiv) and catalytic amount of DMF (2 drops). The reaction was allowed to stir at room temperature for 30 minutes. To the reaction mixture was then added the desired aniline (solid or solution in THF, 128 mg, 1.0 equiv). The reaction was allowed to stir overnight. The solvent removed in vacuo and the crude product was purified by silica gel column chromatography using Combi-Flash system (Hex:EtOAc). The product was obtained as a white solid in 74% (164 mg) yield.

¹H NMR (400 MHz, CDC₁₃) δ 7.88-7.97 (m, 4H), 7.56-7.63 (m, 4H), 7.43 (s, 1H), 3.90 (br s, 2H), 1.70 (br s, 6H), 1.60 (br s, 3H), 1.50 (br s, 9H).

### (S)-2-Amino-N-(4-(2-(4-bromophenyl)thiazol-4-yl)phenyl)-3-hydroxy-2-methylpropanamide

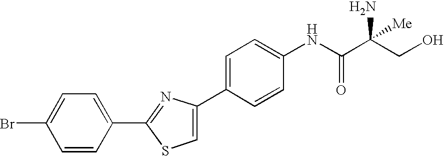

The product was obtained as a white solid in 75% (20.0 mg) yield. MS (ESI, M+H⁺)=432.6 and 434.1; ¹H NMR (400 MHz, DMSO-d₆) δ 10.01 (br s, 1H), 8.18 (br s, 2H), 8.14 (s, 1H), 8.04 (d, 2H, J=8.6 Hz), 7.96 (d, 2H, J=8.6 Hz), 7.70-7.76 (m, 4H), 5.60 (br s, 1H), 4.00 (br d, 1H), 3.65 (br d, 1H), 1.50 (s, 3H).

### (S)-2-Amino-3-hydroxy-2-methyl-N-(4-(2-(4-phenylphenyl)thiazol-4-yl)phenyl)propanamide

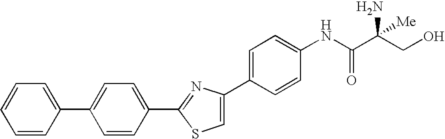

The product was obtained as a white solid in 58% (15.0 mg) yield over two steps. MS (ESI, M+H⁺)=430.4; ¹H NMR (400 MHz, DMSO-d₆) δ 10.01 (br s, 1H), 8.18 (br s, 2H), 8.04-8.14 (m, 5H), 7.84 (d, 2H, J=8.6 Hz), 7.72-7.78 (m, 4H), 7.50 (t, 2H, J=8.6 Hz), 7.37-7.47 (m, 2H), 5.80 (br s, 1H), 4.01 (br d, 1H), 3.65 (br d, 1H), 1.51 (s, 3H).

### (S)-2-Amino-N-(4-(2-(4-(benzo[d]11,3]dioxol-6-yl)phenyl)thiazol-4-yl)phenyl)-3-hydroxy-2-methylpropanamide

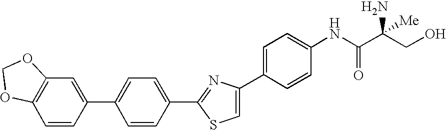

The product was obtained as a white solid in 42% (15.0 mg) yield over two steps. MS (ESI, M+H⁺)=474.3; ¹H NMR (400 MHz, DMSO-d₆) δ 10.05 (br s, 1H), 8.25 (br s, 2H), 7.98-8.11 (m, 5H), 7.76-7.82 (m, 4H), 7.36 (d, 1H, J=1.6 Hz), 7.26 (dd, 1H, J=8.2 Hz, J=2.0 Hz), 7.04 (d, 1H, J=8.2 Hz), 6.09 (s, 2H), 5.05 (br s, 1H), 3.78 (br d, 1H), 3.30 (br d, 1H), 1.20 (s, 3H).

### (S)-2-(4-(2-(4-Bromophenyl)thiazol-4-yl)phenylcarbamoyl)-2-aminopropyl dihydrogen phosphate

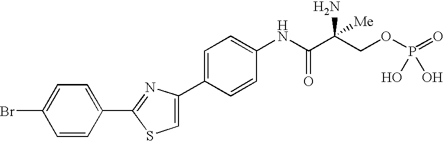

The product was obtained as a white solid in 83% (5.0 mg) yield over two steps. MS (ESI, M+H⁺)=512.6 and 514.3.

### (S)-2-(4-(2-(4-Phenylphenyl)thiazol-4-yl)phenylcarbamoyl)-2-aminopropyl dihydrogen phosphate

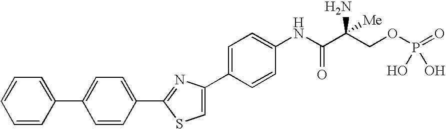

The product was obtained as a white solid in 65% (3.0 mg) yield over two steps. MS (ESI, M+H⁺)=510.2.

### (S)-2-(4-(2-(4-(Benzo[d][1,31dioxol-6-yl)phenyl)thiazol-4-yl)phenylcarbamoyl)-2-aminopropyl dihydrogen phosphate

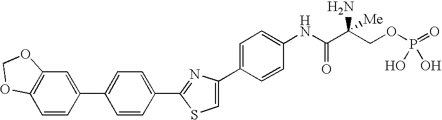

The product was obtained as a white solid in 45% (3.0 mg) yield over two steps. MS (ESI, M+H⁺)=554.1.

Acetophenone-Based Linker:

Synthesis of the acetophenone-based linker was achieved using the process described in Scheme 13. Reaction of protected 4-aminobenzoyl chloride with 4-ethynylbiphenyl followed by hydrogenation of the alkyne provided the Boc-protected 4-aminoacetophenone. Acylation of the amino group after removal of the Boc protecting group afforded an orthogonally protected oxazolidine intermediate, which could be removed using p-TsOH. The free alcohol could then be rapidly converted into the final phosphate product.

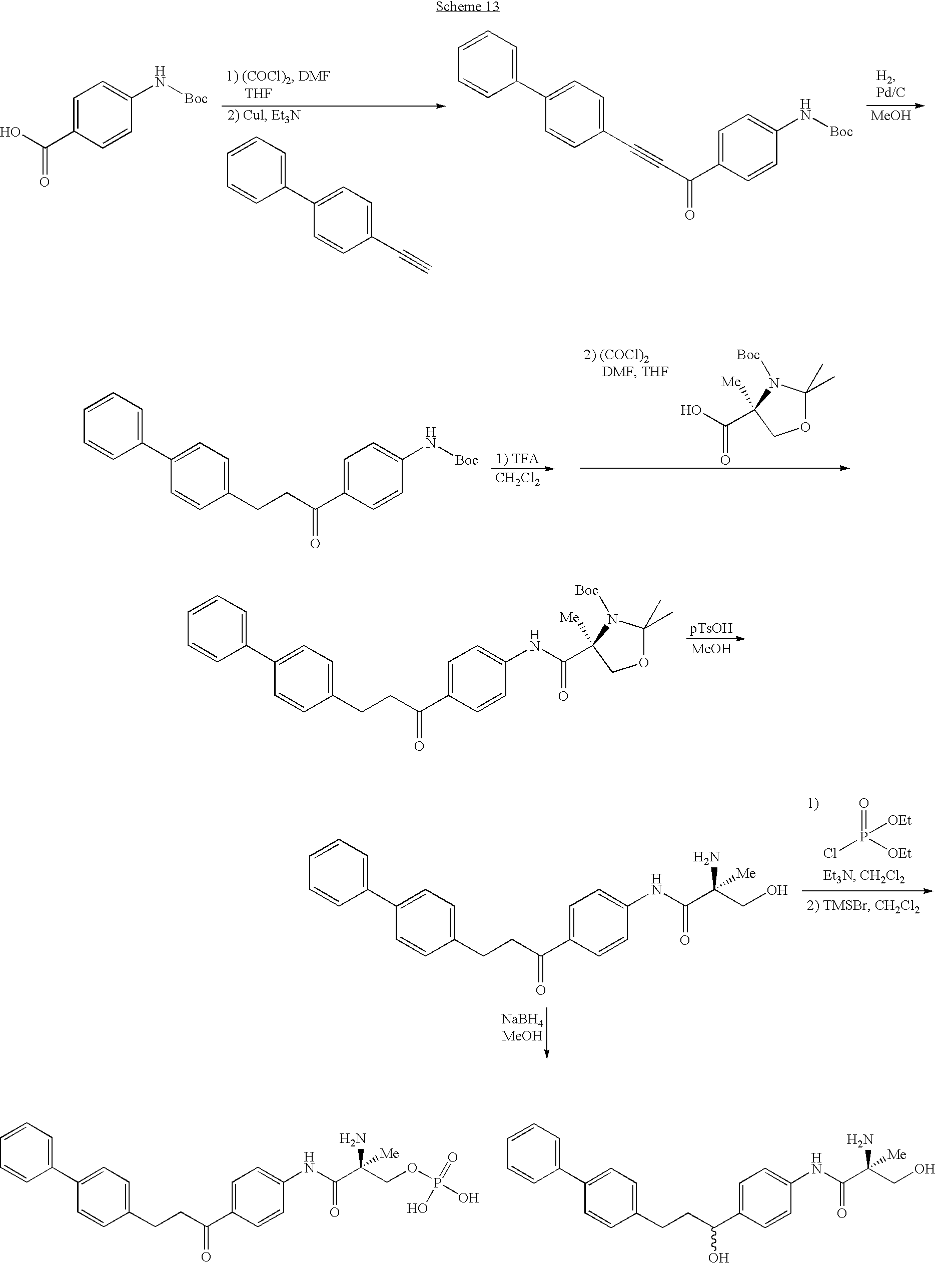

### tert-Butyl 4-(3-(4-phenylphenyl)propanoyl)phenylcarbamate

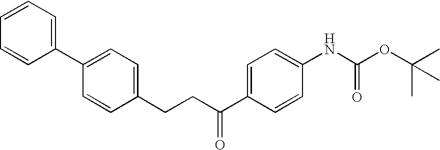

The product was obtained as a yellow solid in 25% (185 mg) yield over three steps. ¹H NMR (400 MHz, CDC₁₃) δ 7.90 (d, 2H, J=8.6 Hz), 7.50-7.60 (m, 4H), 7.40-7.46 (m, 4H), 7.30-7.36 (m, 3H), 6.66 (br s, 1H), 3.29 (t, 2H, J=7.0 Hz), 3.10 (t, 2H, J=7.0 Hz), 1.54 (s, 9H).

### (S)—N-(4-(3-(4-Phenylphenyl)propanoyl)phenyl)-2-amino-3-hydroxy-2-methylpropanamide

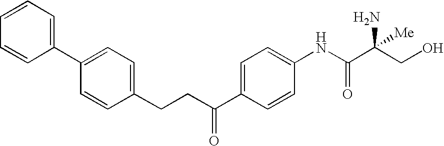

The product was obtained as a white solid in 66% (111 mg) yield over four steps. MS (ESI, M+H⁺)=403.3; ¹H NMR (400 MHz, DMSO-d₆) δ 7.95 (d, 2H, J=8.6 Hz), 7.80 (d, 2H, J=8.6 Hz), 7.52-7.63 (m, 4H), 7.43 (t, 2H, J=8.6 Hz), 7.29-7.38 (m, 3H), 4.99 (br t, 1H, J=5.1 Hz), 3.72 (dd, 1H, J=10.2 Hz, J=5.2 Hz), 3.35 (t, 2H, J=6.8 Hz), 3.20 (dd, 1H, J=10.2 Hz, J=5.2 Hz), 2.96 (t, 2H, J=6.8 Hz), 1.15 (s, 3H).

### (S)-2-(4-(3-(4-Phenylphenyl)propanoyl)phenylcarbamoyl)-2-aminopropyl dihydrogen phosphate

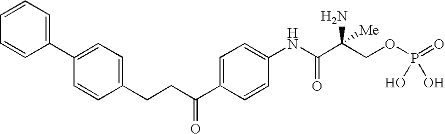

The product was obtained as a white solid in 62% (5.0 mg) yield over two steps. MS (ESI, M+H⁺)=483.5.

### (S)-2-Amino-3-hydroxy-N-(4-(1-hydroxy-3-(4-phenylphenyl)propyl)phenyl)-2-methylpropanamide

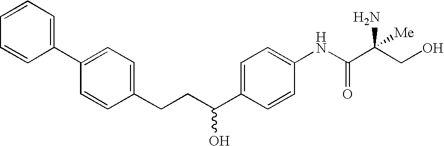

The product was obtained as a white solid in 80% (5.0 mg) yield. MS (ESI, M+H⁺)=405.2.

Thioether Linker:

Synthesis of the thioether, sulfoxide and sulfone linkers was achieved using the process described in Scheme 14. Reduction of biphenyl acetic acid to alcohol followed by conversion of the alcohol to bromo leaving group allowed conversion of the functional group to a thioether. The nitro group was then reduced and acylated to afford oxazolidine intermediate. The thioether could then be further functionalized before deprotection of the Boc and oxazolidine protecting groups. The free alcohol was then converted into the desired final phosphate product.

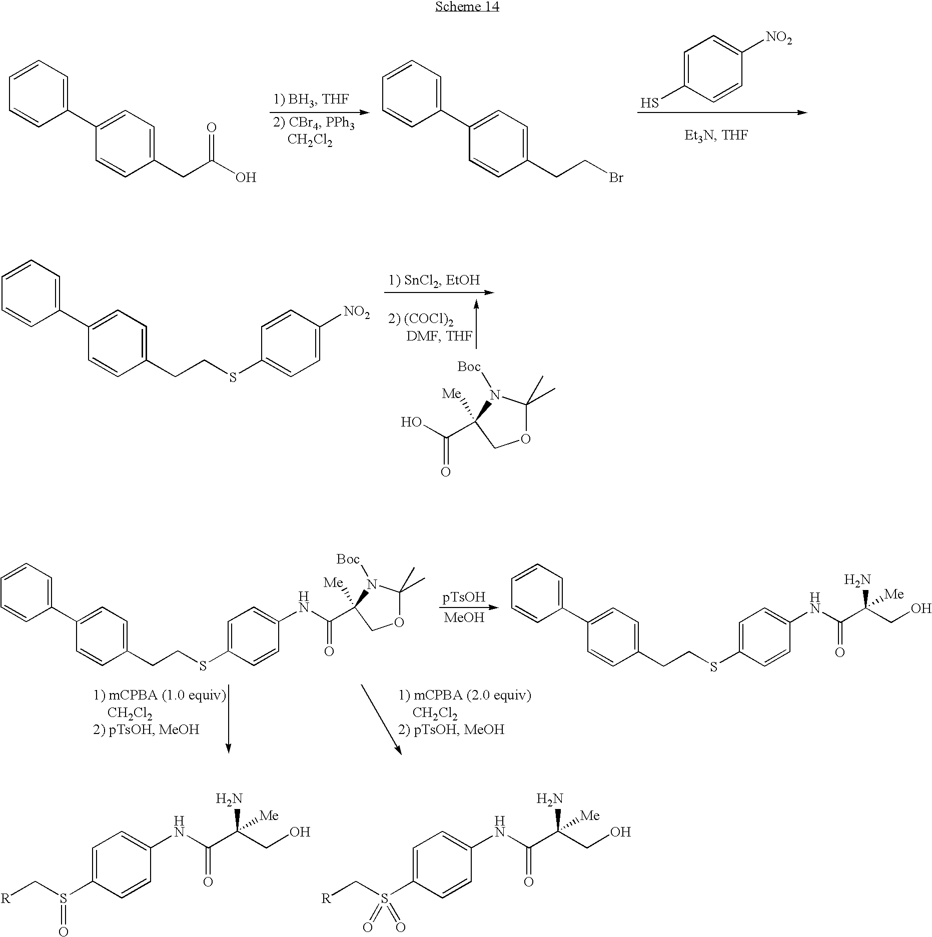

### 4-(2-(4-Nitrophenylthio)ethyl)biphenyl

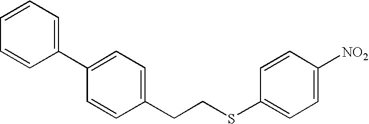

The product was obtained as a yellow solid in 73% (0.72 g) yield over three steps. ¹H NMR (400 MHz, CDC₁₃) δ 8.14 (d, 2H, J=8.6 Hz), 7.53-7.62 (m, 6H), 7.44 (t, 2H, J=8.6 Hz), 7.28-7.38 (m, 3H), 3.32 (t, 2H, J=7.4 Hz), 3.06 (t, 2H, J=7.4 Hz).

### (S)-tert-Butyl 4-(4-(4-phenylphenethylthio)phenylcarbamoyl)-2,2,4-trimethyloxazolidine-3-carboxylate

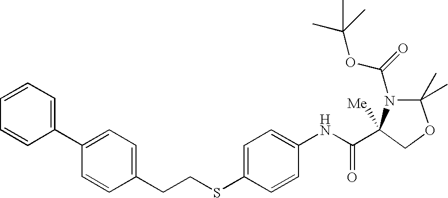

The product was obtained as a white solid in 42% (160 mg) yield over three steps. ¹H NMR (400 MHz, CDC₁₃) δ 7.48-7.52 (m, 2H), 7.45 (d, 2H, J=8.6 Hz), 7.41 (d, 2H, J=8.8 Hz), 7.36 (t, 2H, J=8.6 Hz), 7.22-7.32 (m, 3H), 7.29-7.38 (m, 3H), 7.12 (d, 2H, J=8.8 Hz), 3.70 (br s, 2H), 3.08 (t, 2H, J=7.0 Hz), 2.86 (d, 2H, J=7.0 Hz), 2.96 (t, 2H, J=6.8 Hz), 1.62 (s, 6H), 1.48 (s, 3H), 1.43 (br s, 9H).

### (S)—N-(4-(4-Phenylphenethylthio)phenyl)-2-amino-3-hydroxy-2-methylpropanamide

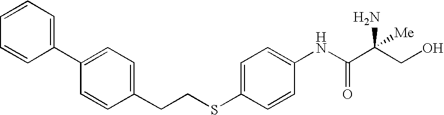

The product was obtained as a white solid in 42% (160 mg) yield over three steps. MS (ESI, M+H⁺)=407.3; ¹H NMR (400 MHz, DMSO-d₆) δ 9.94 (br s, 1H), 8.16 (br s, 2H), 7.54-7.64 (m, 6H), 7.44 (d, 2H, J=8.6 Hz), 7.28-7.40 (m, 5H), 5.76 (br s, 1H), 3.99 (br dd, 2H), 3.63 (br dd, 1H), 3.22 (t, 2H, J=7.0 Hz), 2.87 (t, 2H, J=6.8 Hz), 1.49 (s, 3H).

### (2S)—N-(4-(4-Phenylphenethylsulfinyl)phenyl)-2-amino-3-hydroxy-2-methylpropanamide

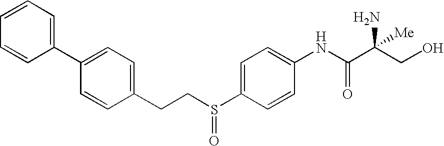

The product was obtained as a white solid in 90% (40 mg) yield over two steps. MS (ESI, M+H⁺)=423.7; ¹H NMR (400 MHz, DMSO-d₆) δ 10.23 (br s, 1H), 8.23 (br s, 2H), 7.88 (d, 2H, J=8.6 Hz), 7.68 (d, 2H, J=8.6 Hz), 7.61 (dd, 2H, J=8.6 Hz, J=1.6 Hz), 7.56 (d, 2H, J=8.6 Hz), 7.43 (t, 2H, J=8.6 Hz), 7.27-7.36 (m, 3H), 5.78 (br s, 1H), 4.05 (br d, 2H), 3.53 (br d, 1H), 3.20-3.43 (m, 1H), 2.90-3.10 (m, 2H), 2.67-2.78 (m, 1H0, 1.48 (s, 3H).

### (S)—N-(4-(4-Phenylphenethylsulfonyl)phenyl)-2-amino-3-hydroxy-2-methylpropanamide

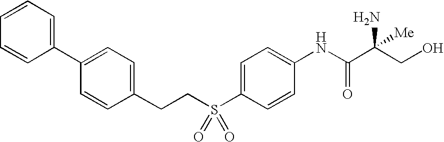

The product was obtained as a white solid in 91% (52 mg) yield over two steps. MS (ESI, M+H⁺)=439.4; ¹H NMR (400 MHz, DMSO-d₆) δ 10.41 (br s, 1H), 8.25 (br s, 2H), 7.86-7.99 (m, 4H), 7.59 (d, 2H, J=8.6 Hz), 7.52 (d, 2H, J=8.6 Hz), 7.42 (t, 2H, J=8.6 Hz), 7.32 (tt, 1H, J=8.4 Hz, J=1.2 Hz), 7.27 (d, 2H, J=8.6 Hz), 5.80 (br t, 1H), 4.06 (dd, 1H, J=11.6 Hz, J=4.7 Hz), 3.58-3.69 (m, 3H), 2.85-2.93 (m, 2H), 1.52 (s, 3H).

### (S)-2-(4-(4-Phenylphenethylthio)phenylcarbamoyl)-2-aminopropyl dihydrogen phosphate

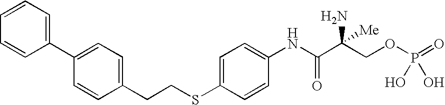

The product was obtained as a white solid in 65% (6.0 mg) yield over two steps. MS (ESI, M+H⁺)=487.3.

### (S)-2-(4-(4-Phenylphenethylsulfinyl)phenylcarbamoyl)-2-aminopropyl dihydrogen phosphate:

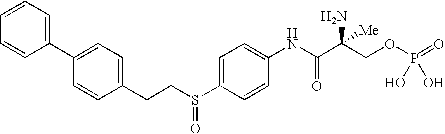

The product was obtained as a white solid in 45% (1.5 mg) yield over two steps. MS (ESI, M+H⁺)=503.1.

### (S)-2-(4-(4-Phenylphenethylsulfonyl)phenylcarbamoyl)-2-aminopropyl dihydrogen phosphate

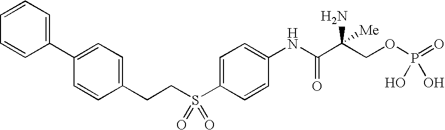

The product was obtained as a white solid in 65% (15.0 mg) yield over two steps. MS (ESI, M+H⁺)=519.7.

Benzamide Linker:

The benzamide linker based compounds were synthesized as described in Scheme 15. Acylation of 4-phenylbenzylamine followed by a one-pot, two step acylation of the aniline intermediate afforded orthogonally protected oxazolidine intermediate. The oxazolidine intermediate was then converted into free alcohol and its phosphate respectively.

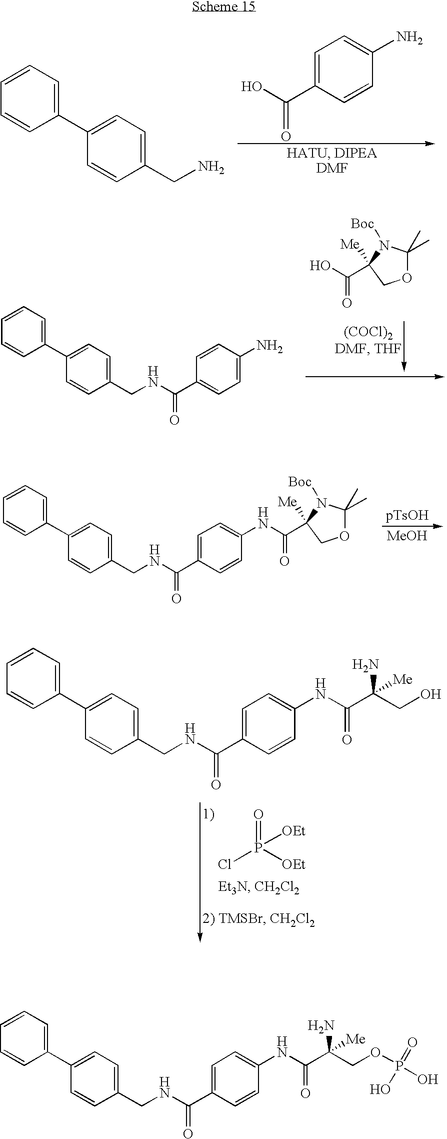

### N-(4-Phenylbenzyl)-4-aminobenzamide

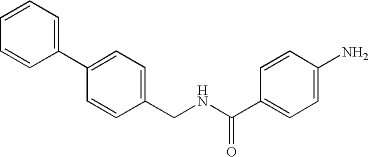

The product was obtained as a yellow solid in 60% (0.49 g) yield. ¹H NMR (400 MHz, CDC₁₃) δ 7.64 (d, 2H, J=8.6 Hz), 7.55-7.60 (m, 4H), 7.40-7.47 (m, 4H), 7.35 (tt, 1H, J=8.6 Hz, J=1.2 Hz), 6.66 (d, 2H, J=8.6 Hz), 6.25 (br t, 1H), 4.67 (d, 2H, J=5.9 Hz), 3.95 (br s, 2H).

### (S)-tert-Butyl 4-(4-(4-phenylbenzylcarbamoyl)phenylcarbamoyl)-2,2,4-trimethyloxazolidine-3-carboxylate

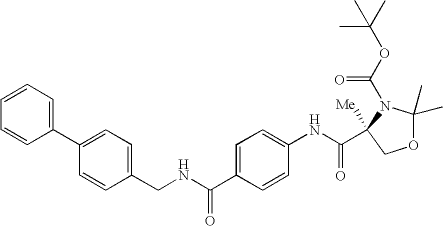

The product was obtained as a white solid in 43% (105 mg) yield. ¹H NMR (400 MHz, CDC₁₃) δ 7.79 (d, 2H, J=8.8 Hz), 7.55-7.63 (m, 6H), 7.41-7.47 (m, 4H), 7.35 (tt, 1H, J=8.6 Hz, J=1.2 Hz), 6.37 (br t, 1H), 4.69 (d, 2H, J=5.5 Hz), 3.78 (br s, 2H), 1.69 (s, 6H), 1.59 (s, 3H), 1.48 (br s, 9H).

### (S)—N-(4-(N′—(4-Phenylbenzyl)formamido)phenyl)-2-amino-3-hydroxy-2-methylpropanamide

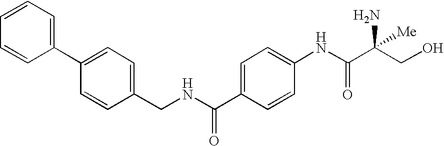

The product was obtained as a white solid in 61% (35 mg) yield. MS (ESI, M+H⁺)=404.3; ¹H NMR (400 MHz, DMSO-d₆) δ 8.98 (br 1, 1H, J=5.8 Hz), 7.87 (d, 2H, J=8.6 Hz), 7.77 (d, 2H, J=8.6 Hz), 7.60-7.66 (m, 4H), 7.32-7.48 (m, 5H), 7.10 (br d, 1H), 5.02 (br t, 1H), 4.50 (d, 2H, J=5.8 Hz), 3.75 (dd, 1H, J=10.5 Hz, J=5.5 Hz), 3.22 (dd, 1H, J=10.5 Hz, J=5.1 Hz), 1.17 (s,3H).

### (S)-2-(4-(N′—(4-Phenylbenzyl)formamido)phenylcarbamoyl)-2-aminopropyl dihydrogen phosphate

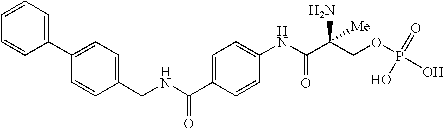

The product was obtained as a white solid in 30% (7.0 mg) yield over two steps. MS (ESI, M+H⁺)=484.7.

Biphenyl Ethanol Linker:

A number of substituted biphenyl ethanols were synthesized using a Suzuki cross-coupling protocol a described in Scheme 16.

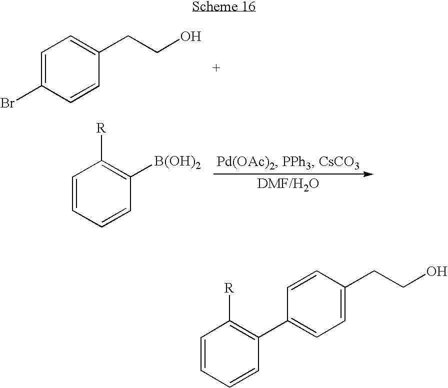

Reaction of the substituted biphenyl ethanol with substituted 4-fluoro-nitrobenzene under Williamson ether synthesis (scheme 17) conditions followed by hydrogenation and coupling with amino acid provided the Boc protected amino-alcohol which was further phosphorylated or deprotected to obtain the desired final product. 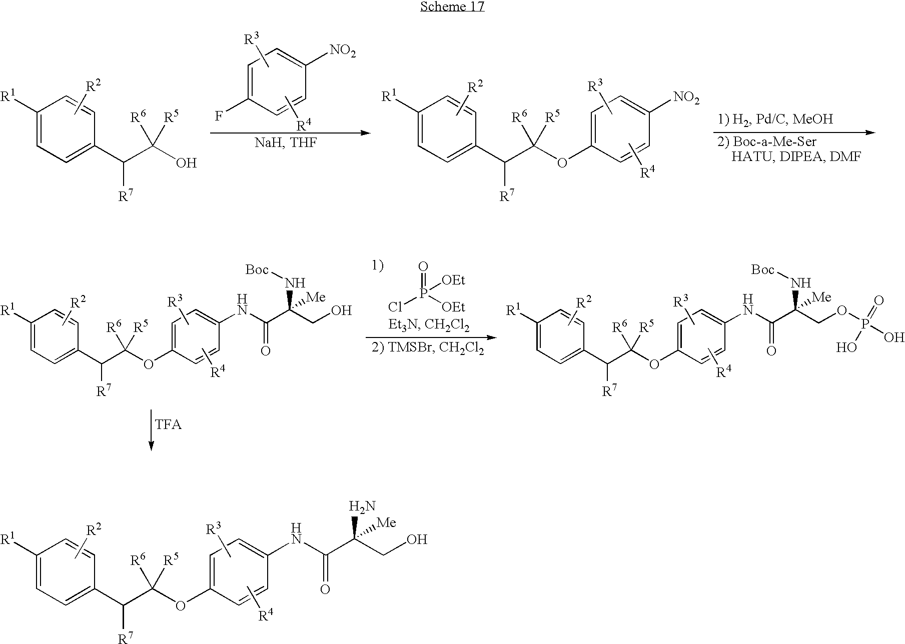
General Procedure for Synthesis of Substituted Biaryl Ethanol.

To a DMF solution of the 4-(haloaryloxy)-aniline (1.0 equiv) and substituted aryl boronic acid in a microwave tube, was added Pd(OAc)₂ (0.1 equiv), triphenyl phosphine (0.2 equiv), cesium carbonate (1.0-1.5 equiv) and TBAC (0.1 equiv). The reaction was then sealed and heated at 50-70° C. for 3-18 hours using an oil bath. The reaction mixture was diluted with EtOAc (25 mL), washed with water (2×10 mL) and then brine (1×10 mL). The organic layer was then dried over MgSO₄, and then solvent removed under reduced pressure. The crude product was purified using the Combi-Flash silica gel column chromatography, using a Hexane/EtOAc gradient.

### 2-(2′-Methyl-biphenyl-4-yl)-ethanol

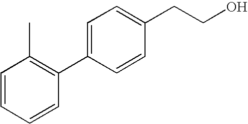

The final product was obtained as a white solid after column chromatography, in 85% yield. ¹H NMR (400 MHz, CDC₁₃) δ 7.28 (s, 4H), 7.26 (s, 4H), 3.93 (t, 2H, J=6.4 Hz), 2.93 (t, 2H, J=6.4 Hz), 2.28 (s, 3H).

### 2-(2′—Chloro-biphenyl-4-yl)-ethanol

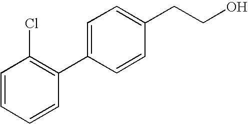

The final product was obtained as a white solid after column chromatography, in 85% yield. ¹H NMR (400 MHz, CDC₁₃) δ 7.45-7.48 (m, 1H), 7.405 (d, 2H, J=8.0 Hz), 7.28-7.33 (m, 4H), 3.93 (t, 2H, J=6.4 Hz), 2.94 (t, 2H, J=6.4 Hz).

### 2-(2-Cyano-biphenyl-4-yl)-ethanol

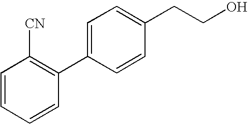

The final product was obtained as a white solid after column chromatography, in 97% yield. ¹H NMR (400 MHz, CDC₁₃) δ 7.76 (dd, 1H, J=8.0 and 1.2), 7.64 (m, 1H), 7.49-7.53 (m, 3H), 7.43 (m, 1H), 7.36 (d, 2H, J=8.0 Hz), 3.93 (t, 2H, J=6.8 Hz), 2.95 (t, 2H, J=6.4 Hz).

### 2-Methyl-4′[2-(4-nitro-phenoxy)-ethyl]-biphenyl

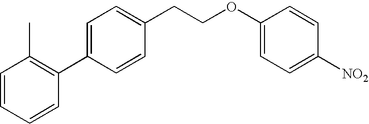

The final product was obtained as a yellow solid after column chromatography, in 88% yield. ¹H NMR (400 MHz, CDC₁₃) δ 8.20 (d, 2H, J=9.2 Hz), 7.2-7.316 (m, 8H), 6.97 (d, 2H, J=8.8 Hz), 4.32 (t, 2H, J=7.2 Hz), 3.19 (t, 2H, J=6.8 Hz), 2.27 (s, 3H).

### 2-Chloro-4′[2-(4-nitro-phenoxy)-ethyl]-biphenyl

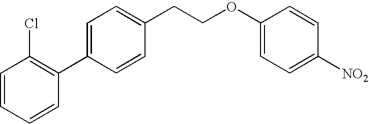

The final product was obtained as a yellow solid after column chromatography, in 88% yield. ¹H NMR (400 MHz, CDC₁₃) δ 8.20 (d, 2H, J=9.2 Hz), 7.41-7.48 (m, 3H), 7.27-7.36 (m, 4H), 7.24 (s, 1H), 6.97 (d, 2H, J=9.2 Hz), 4.32 (t, 2H, J=6.8 Hz), 3.20 (t, 2H, J=6.8 Hz).

### 4′-[2-(4-Nitro-phenoxy)-ethyl]-biphenyl-2-carbonitrile

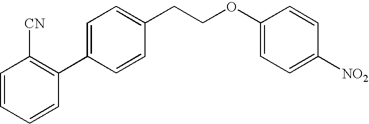

The final product was obtained as an off white solid after column chromatography, in 81% yield. ¹H NMR (400 MHz, CDC₁₃) δ 8.19 (d, 2H, J=9.2 Hz), 7.76 (dd, 1H, J=8.0 and 1.2), 7.66-7.62 (m, 1H), 7.54-7.49 (m, 3H), 7.40-7.46 (m, 3H), 6.96 (d, 2H, J=9.2 Hz), 4.31 (t, 2H, J=6.8 Hz), 3.21 (t, 2H, J=6.8 Hz).

### 4-[2-(2-Chloro-4-nitro-phenoxy)-ethyl]biphenyl

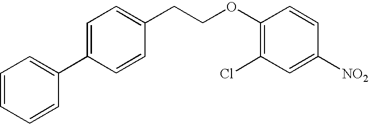

The final product was obtained as a yellow solid after column chromatography, in 50% yield. ¹H NMR (400 MHz, CDC₁₃) δ 8.29 (d, 1H, J=3.0 Hz), 8.13 (dd, 1H, J=2.8 and 9.2 Hz), 7.57-7.60 (m, 4H), 7.40-7.46 (m, 4H), 7.35 (m, 1H), 6.95 (d, 2H, J=9.2 Hz), 4.35 (t, 2H, J=6.4 Hz), 3.25 (t, 2H, J=6.8 Hz).

### 4-[2-(2-Methyl-4-nitro-phenoxy)-ethyl]biphenyl

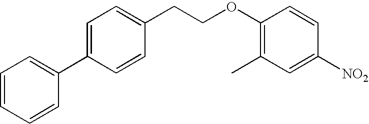

The final product was obtained as a yellow solid after column chromatography, in 78% yield. ¹H NMR (400 MHz, CDC₁₃) δ 8.03-8.09 (m, 2H), 7.55-7.59 (m, 4H), 7.44 (t, 2H, J=8.0), 7.37 (d, 2H, J=8.4 Hz), 6.84 (d, 2H, J=9.2 Hz), 4.30 (t, 2H, J=6.4 Hz), 3.20 (t, 2H, J=6.8 Hz), 2.27 (s, 3H).

### 4-(2-Biphenyl-4-ylethoxy)-3-chloro-phenylamine

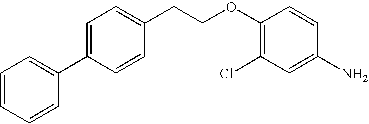

The final product was obtained as a brown oil after column chromatography, in 79% yield. ¹H NMR (400 MHz, CDC₁₃) δ 7.54-7.60 (m, 5H), 7.46-7.38 (m, 4H), 6.75 (m, 2H), 6.52 (dd, 1H, J=2.8 and 8.8), 4.17 (t, 2H, J=7.6 Hz), 3.15 (t, 2H, J=7.2 Hz).

### 4-(2-Biphenyl-4-ylethoxy)-3-methyl-phenylamine

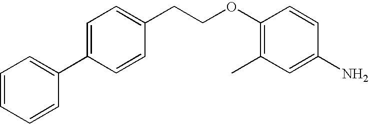

The final product was obtained as an off white solid after column chromatography, in 84% yield. ¹H NMR (400 MHz, CDC₁₃) δ 7.51-7.58 (m, 4H), 7.39-7.43 (m, 2H), 7.34 (d, 3H, J=8.4 Hz), 6.64 (d, 1H, J=8.8 Hz), 6.50 (d, 1H, J=3.2 Hz), 6.50 (dd, 1H, J=2.8 and 8.4), 4.10 (t, 2H, J=7.2 Hz), 3.09 (t, 2H, J=7.2 Hz), 2.13 (s, 3H).

### (2-Hydroxy-l-methyl-1-{4-[2-(2′-methyl-biphenyl-4-yl)-ethoxy]-phenyl carbamoyl}-ethyl)-carbamic acid tert-butyl ester

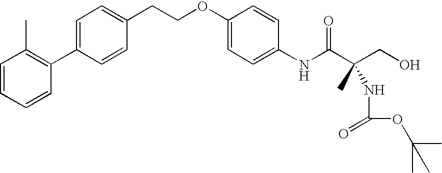

The final product was obtained as an off white solid after column chromatography, in 73% yield. ¹H NMR (400 MHz, CDC₁₃) ; 7.41 (dd, 2H, J=6.4 and 9.4 Hz), 7.31-7.33 (m, 2H), 7.28 (s, 2H), 7.22-7.26 (m, 4H), 6.88 (d, 1H, J=8.8 Hz), 4.2 (t, 2H, J=7.2 Hz), 3.78 (d, 1H, J=12.0 Hz), 3.56 (d, 1H, J=10.8 Hz), 3.13 (t, 2H, J=7.2 Hz), 2.28 (s, 3H), 1.58 (s, 3H), 1.46 (s, 9H).

### (1-{4-[2-(2′—Chloro-biphenyl-4-yl)-ethoxy]-phenyl carbamoyl}-2-hydroxy-1-methyl-ethyl)-carbamic acid tert-butyl ester

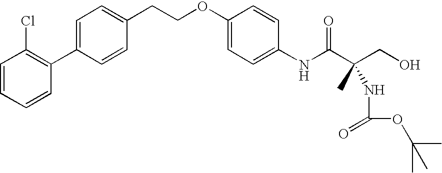

The final product was obtained as an off white oil after column chromatography, in 83% yield. ¹H NMR (400 MHz, CDC₁₃) δ 7.55 (m, 1H), 7.46 (dd, 1H, J=7.2 and 8.8 Hz), 7.37-7.43 (m, 3H), 7.27-7.35 (m, 4H), 7.26 (s, 1H), 6.88 (d, 2H, J=9.2 Hz), 4.21 (t, 2H, J=7.2 Hz), 4.08 (br.s, 1H), 3.557 (d, 1H, J=10.8 Hz), 3.14 (t, 2H, J=7.2 Hz), 1.58 (s, 3H), 1.46 (s, 9H).

### {1-[4-(2-Biphenyl-4-yl-ethoxy)-3-chloro-phenylcarbamoyl]-2-hydroxy-1-methyl-ethyl}-carbamic acid tert-butyl ester

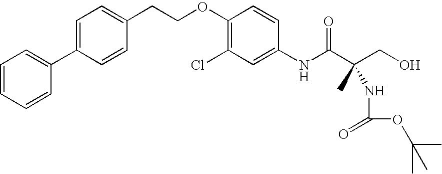

The final product was obtained as an off white solid after column chromatography, in 85% yield. ¹H NMR (400 MHz, CDC₁₃) δ 7.63 (s, 1H), 7.53-7.59 (m, 4H), 7.38-7.44 (m, 4H), 7.30-7.35 (m, 2H), 6.84 (d, 1H, J=8.8 Hz), 4.21 (t, 2H, J=7.2 Hz), 4.06 (br.s, 1H), 3.6 (s, 1H), 3.17 (t, 2H, J=7.2 Hz), 1.56 (s, 3H), 1.44 (s, 9H).

### {1-[4-(2-Biphenyl-4-yl-ethoxy)-3-methyl-phenylcarbamoyl]-2-hydroxy-1-methyl-ethyl)-carbamic acid tert-butyl ester

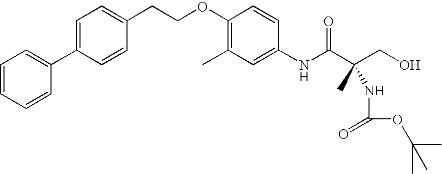

The final product was obtained as an off white solid after column chromatography, in 81% yield. ¹H NMR (400 MHz, CDC₁₃) δ 7.53-7.59 (m, 4H), 7.43 (t, 2H, J=7.6 Hz), 7.36 (d, 3H, J=8.4 Hz), 7.26 (br. s, 2H), 6.76 (d, 1H, J=8.4 Hz), 4.18 (t, 2H, J=6.4 Hz), 3.56 (br.s, 1H), 3.31 (s, 1H), 3.14 (t, 2H, J=6.8 Hz), 2.19 (s, 3H), 1.57 (s, 3H), 1.46 (s, 9H).

### tert-Butyl (S)-2-(4-(4-phenylphenethyloxy)-3-(methylformyl)phenylcarbamoyl)-1-hydroxypropan-2-ylcarbamate

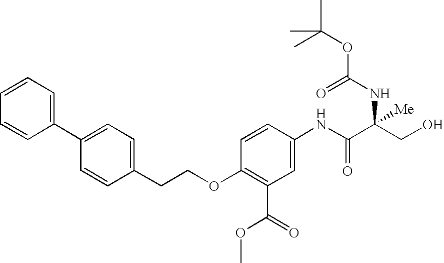

¹H NMR (400 MHz, CDC₁₃) δ 7.87 (d, 1H, J=2.7 Hz), 7.65 (dd, 1H, J=8.8 Hz, J=2.7 Hz), 7.50-7.60 (m, 4H), 7.28-7.46 (m, 5H), 6.83 (d, 1H, J=8.8 Hz), 5.59 (br s, 1H), 4.53 (br t, 1H), 4.25 (t, 2H, J=6.8 Hz), 3.87 (s, 3H), 3.53-3.62 (m, 1H), 3.18 (t, 2H, J=6.8 Hz), 3.16-3.18 (m, 1H), 1.57 (s, 3H), 1.47 (s, 9H).

### tert-Butyl (S)-2-(4-(4-phenyl)phenethyloxy)-3-(trifluoromethyl) phenylcarbamoyl)-1-hydroxypropan-2-ylcarbamate

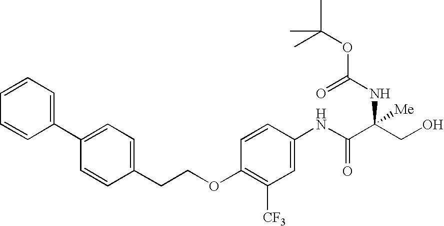

The product was obtained as a thick colorless oil in 45% (300 mg) yield over two steps from 2-biphenylethanol. ¹H NMR (400 MHz, CDC₁₃) δ 9.70 (br s, 1H), 7.70 (d, 1H, J=2.7 Hz), 7.64 (dd, 1H, J=8.8 Hz, J=2.7 Hz), 7.52-7.60 (m, 4H), 7.30-7.46 (m, 5H), 6.94 (d, 1H, J=8.8 Hz), 5.60 (br s, 1H), 4.25 (t, 2H, J=6.8 Hz), 4.04-4.14 (m, 1H), 3.50-3.60 (m, 1H), 3.17 (t, 2H, J=6.8 Hz), 1.57 (s, 3H), 1.47 (s, 9H).

### tert-Butyl (S)-2-(4-(4-phenylphenethyloxy)-3-bromophenylcarbamoyl)-1-hydroxypropan-2-ylcarbamate

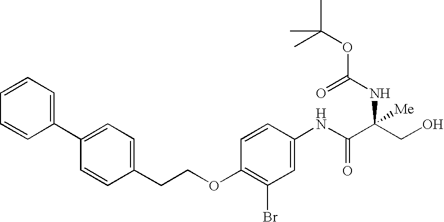

The product was obtained as a thick colorless oil in 40% (385 mg) yield over two steps from 2-biphenylethanol. ¹H NMR (400 MHz, CDC₁₃) δ 9.60 (br s, 1H), 7.7 8 (d, 1H, J=2.3 Hz), 7.53-7.62 (m, 5H), 7.30-7.46 (m, 5H), 6.83 (d, 1H, J=8.8 Hz), 5.60 (br s, 1H), 4.22 (t, 2H, J=6.8 Hz), 4.06-4.12 (m, 1H), 3.58 (br d, 1H), 3.20 (t, 2H, J=6.8 Hz), 1.58 (s, 3H), 1.46 (s, 9H).

### 2-Amino-3-hydroxy-2-methyl-N-{4-[2-(2′-methyl-biphenyl-4-yl)-ethoxy]-phenyl}-propionamide

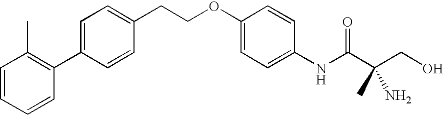

The compound was obtained as a white solid after HPLC purification. Yield: 93%, (92 mg). MS (ESI, M+H⁺)=404.4

### 2-Amino-N-{4-[2-(2′—Chloro-biphenyl-4-yl)-ethoxy]-phenyl}-3-hydroxy-2-methyl-propionamide

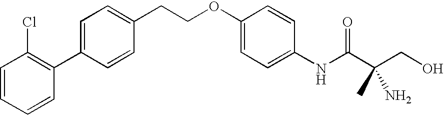

The compound was obtained as a white solid after HPLC purification. Yield: 84%, (68 mg). MS (ESI, M+H⁺)=425.7

### (1-{4-[2-(2′—Cyano-biphenyl-4-yl)-ethoxy]-phenylcarbamoyl}-2-hydroxy-1-methyl-ethyl)-carbamic acid tert-butyl ester

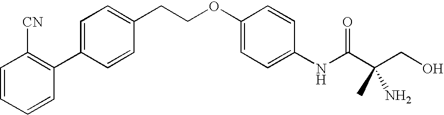

The final product was obtained as an off white oil after column chromatography, in 80% yield. MS (ESI, M+H⁺)=416.6,¹H NMR (400 MHz, CDC₁₃) δ 7.55-7.63 (m, 3H), 7.53 (br. s, 2H), 7.48-7.51 (m, 4H), 6.97 (d, 2H, J=9.2 Hz), 5.77 (t, 1H, J=5.2 Hz), 4.24 (t, 2H, J=6.8 Hz), 3.96 (dd, 1H, J=12.0 and 5.2 Hz), 3.628 (dd, 1H, J=11.6 and 4.8 Hz), 3.12 (t, 2H, J=6.8 Hz), 1.47 (s, 3H).

### (S)—N-(4-(4-Phenylphenethyloxy)-3-(trifluoromethyl)phenyl)-2-amino-3-hydroxy-2-methylpropanamide

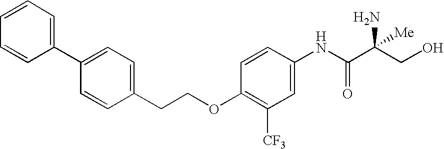

The product was obtained as a white solid in 70% (66 mg) yield. MS (ESI, M+H⁺)=459.7; ¹H NMR (400 MHz, DMSO-d₆) δ 10.01 (br s, 1H), 8.180 (br s, 2H), 7.89 (d, 1H, J=2.4 Hz), 7.82 (dd, 1H, J=8.8 Hz, J=2.4 Hz), 7.58-7.67 (m, 4H), 7.30-7.49 (m, 6H), 5.80 (br s, 1H), 4.32 (t, 2H, J=6.7 Hz), 3.95 (br d, 1H), 3.62 (br d, 1H), 3.09 (t, 2H, J=6.7 Hz), 1.48 (s, 3H).

### (S)—N-(4-(4-phenylphenethyloxy)-3-bromophenyl)-2-amino-3-hydroxy-2-methylpropanamide

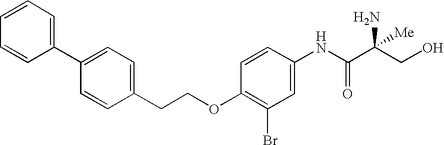

The product was obtained as a white solid in 60% (50 mg) yield. MS (ESI, M+H⁺)=469.4 and 471.4.

### (S)—N-(4-(4-(4-Ethylphenyl)phenethyloxy)phenyl)-2-amino-3-hydroxy-2-methylpropanamide

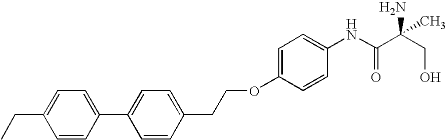

MS (ESI, M+H⁺)=419; ¹H NMR (400 MHz, DMSO-d₆) δ 9.74 (bs, 1H), 8.1 (bs, 1H), 7.55 (m, 4H), 7.47 (d, 2H), 7.37 (d, 2H), 7.26 (d, 2H), 6.93 (d, 2H), 5.74 (bs, 1H), 4.16 (t, 2H), 3.95 (bd, 1H), 3.6 (bd, 1H), 3.04 (t, 2H), 2.6 (q, 2H), 1.73 (m, 4H), 1.45 (s, 3H), 1.19 (t, 3H).

### (S)—N-(4-(4-(4-Trifluoromethylphenyl)phenethyloxy)phenyl)-2-amino-3-hydroxy-2-methylpropanamide

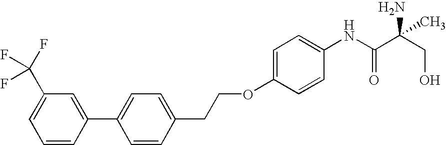

MS (ESI, M+H⁺)=459; ¹H NMR (400 MHz, DMSO-d₆) δ 9.74 (br s, 1H), 8.1 (br s, 2H), 7.95 (m, 2H), 7.68 (m, 3H), 7.47 (m, 3H), 6.93 (m, 2H), 5.74 (br s, 1H), 4.19 (t, 2H), 3.95 (m, 1H), 3.6 (m, 1H), 3.04 (t, 2H), 2.6 (q, 2H), 1.45 (s, 3H).

### (S)—N-(4-(4-(4-ethoxyphenyl)phenethyloxy)phenyl)-2-amino-3-hydroxy-2-methylpropanamide

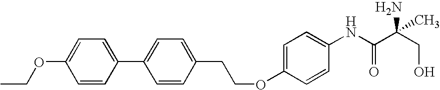

MS (ESI, M+H⁺)=435; ¹H NMR (400 MHz, DMSO-d₆) δ 9.73 (bs, 1H), 8.10 (bs, 2H), 7.57 (m, 2H), 7.46 (m, 2H), 7.32 (t, 1H), 7.22 (m, 1H), 6.94 (m, 2H), 5.75 (t, 1H), 4.19 (t, 2H), 4.04 (q, 2H), 3.93 (m, 1H), 3.61 (m, 1H), 3.07 (t, 2H), 1.45 (s, 3H), 1.32 (t, 3H).

### (S)—N-(4-(4-(4-Chlorophenyl)phenethyloxy)phenyl)-2-amino-3-hydroxy-2-methylpropanamide trifluoroacetic acid salt

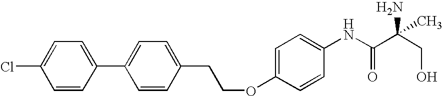

MS (ESI, M+H⁺)=424; ¹H NMR (400 MHz, DMSO-d₆) δ 9.73 (s, 1H), 8.10 (bs, 2H), 7.69 (m, 2H), 7.61 (s, 1H), 7.49 (m, 3H), 7.37 (t, 1H), 7.33 (m, 1H), 6.94 (d, 2H), 5.75 (t, 1H), 4.20 (t, 2H), 4.04 (q, 2H), 3.93 (m, 1H), 3.61 (m, 1H), 3.08 (t, 2H), 1.45 (s, 3H).

### (S)—N-(4-(4-(4-Isopropylphenyl)phenethyloxy)phenyl)-2-amino-3-hydroxy-2-methylpropanamide

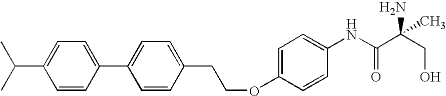

MS (ESI, M+H⁺)=433; ¹H NMR (400 MHz, DMSO-d₆) δ 9.73 (s, 1H), 8.11 (br s, 2H), 7.59 (d, 1H), 7.41-7.34 (m, 3H), 7.2 (d, 1H), 6.9 (d, 2H), 5.65 (br s, 1H), 4.18 (t, 2H), 3.93 (d, 1H), 3.61 (d, 1H), 3.04 (t, 2H), 2.95 (q, 1H), 1.45 (s, 3H), 1.24 (d, 6H).

### (S)—N-(4-(2-(4-Phenyl-3-fluorophenyl)propoxy)phenyl)-2-amino-3-hydroxy-2-methylpropanamide

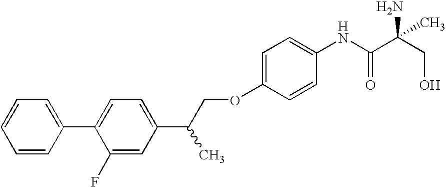

MS (ESI, M+H⁺)=423; ¹H NMR (400 MHz, DMSO-d₆) δ 9.74 (br s, 1H), 8.1 (br s, 1H), 7.5 (m, 6H), 7.40 (m, 2H), 7.28 (m, 2H), 6.93 (d, 2H), 5.74 (br s, 1H), 4.1-4.0 (m, 2H), 3.9 (m, 1H), 3.65 (m, 1H), 3.28 (m, 2H), 1.47 (s, 3H), 1.33 (d, 3H).

### (S)—N-(4-(4-(Thiophen-2-yl)phenethyloxy)phenyl)-2-amino-3-hydroxy-2-methylpropanamide

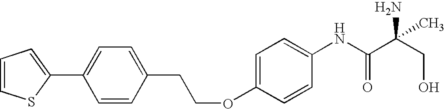

MS (ESI, M+H⁺)=397; ¹H NMR (400 MHz, DMSO-d₆) δ 9.74 (br s, 1H), 8.1 (br s, 2H), 7.60 (d, 2H), 7.50 (m, 4H), 7.36 (d, 2H), 7.12 (m, 1H), 6.95 (d, 2H), 5.74 (br s, 1H), 4.18 (t, 2H), 3.95 (br d, 1H), 3.6 (br d, 1H), 3.04 (t, 2H), 1.45 (s, 3H).

### (S)—N-(4-(4-(3,5-Dimethylisoxazol-4-yl)phenethyloxy)phenyl)-2-amino-3-hydroxy-2-methylpropanamide

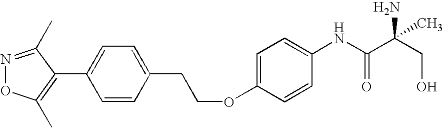

MS (ESI, M+H⁺)=410; ¹H NMR (400 MHz, DMSO-d₆) δ 9.75 (br s, 1H), 8.13 (br s, 2H), 7.50 (d, 2H), 7.41 (d, 2H), 7.3 (d, 2H), 6.9 (d, 2H), 4.22 (t, 2H), 3.94 (d, 1H), 3.6 (d, 1H), 3.07 (t, 2H), 2.4 (s, 3H), 2.2 (s, 2H), 1.48 (s, 3H).

### (S)—N-(4-(4-(Furan-3-yl)phenethyloxy)phenyl)-2-amino-3-hydroxy-2-methylpropanamide

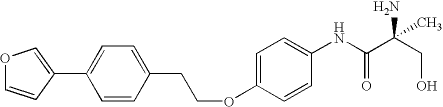

MS (ESI, M+H⁺)=424; ¹H NMR (400 MHz, DMSO-d₆) δ 9.73 (s, 1H), 8.10 (br s, 2H), 7.69 (m, 2H), 7.61 (s, 1H), 7.49 (m, 3H), 7.37 (t, 1H), 7.33 (m, 1H), 6.94 (d, 2H), 5.75 (t, 1H), 4.20 (t, 2H), 4.04 (q, 2H), 3.93 (m, 1H), 3.61 (m, 1H), 3.08 (t, 2H), 1.45 (s, 3H).

### (S)—N-(4-(4-(3-Phenyl)phenethyloxy)phenyl)-2-amino-3-hydroxy-2-methylpropanamide

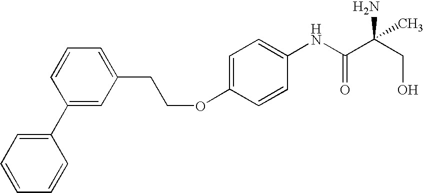

MS (ESI, M+H⁺)=391; ¹H NMR (400 MHz, DMSO-d₆) δ 9.73 (s, 1H), 8.10 (br s, 2H), 7.66 (d, 2H), 7.61 (s, 1H), 7.55-7.30 (m, 4H), 6.94 (d, 2H), 5.75 (bs, 1H), 4.25 (t, 2H), 3.93 (d, 1H), 3.65 (d, 1H), 3.08 (t, 2H), 1.45 (s, 3H).

### (S)—N-(4-(4-(Pyridin-4-yl)phenethyloxy)phenyl)-2-amino-3-hydroxy-2-methylpropanamide

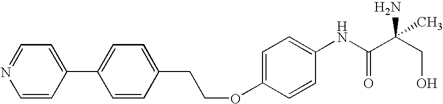

MS (ESI, M+H⁺)=392; ¹H NMR (400 MHz, DMSO-d₆) δ 9.74 (br s, 1H), 8.67 (br s), 8.19 (br s, 2H), 8.12 (br s, 2H), 7.8 (m, 2H), 7.5 (m, 4H), 6.9 (m, 2H), 6.95 (d, 2H), 5.74 (br s, 1H), 4.2 (t, 2H), 3.95 (br d, 1H), 3.04 (t, 2H), 1.45 (s, 3H).

### (S)—N-(4-(4-(Pyridin-3-yl)phenethyloxy)phenyl)-2-amino-3-hydroxy-2-methylpropanamide

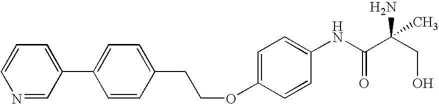

MS (ESI, M+H⁺)=392; ¹H NMR (400 MHz, DMSO-d₆) δ 9.69 (s, 1H), 9.0 (s, 1H), 8.65 (m, 1H), 8.3 (d, 2H), 8.07 (br s, 2H), 7.75 (m, 2H), 7.50 (m, 4H), 6.95 (d, 2H), 4.2 (t, 2H), 3.95 (d, 1H), 3.6 (d, 2H), 3.1 (t, 2H), 1.45 (s, 3H).

### (S)-Phosphoric acid mono-(2-amino-2-{4-[2-(2′-methyl-biphenyl-4-yl)-ethoxy]-phenylcarbamoyl}-propyl) ester

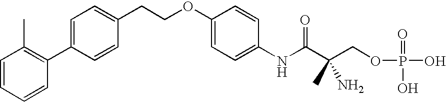

The compound was obtained as a white solid after HPLC purification. Yield: 65%, (41 mg). MS (ESI, M+H⁺)=485.5

### (S)-Phosphoric acid mono-(2-amino-2-{4-[2-(2′-chloro-biphenyl-4-yl)-ethoxy]-phenylcarbamoyl}-propyl) ester

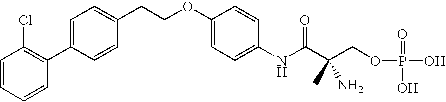

The compound was obtained as a white solid after HPLC purification. Yield: 79%, (25 mg). MS (ESI, M+H⁺)=505.2

### (S)-Phosphoric acid mono-(2-amino-2-{4-[2-(2′-cyano-biphenyl-4-yl)-ethoxy]-phenylcarbamoyl}-propyl) ester

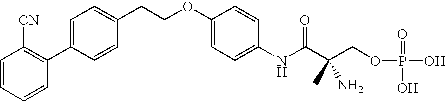

The compound was obtained as a white solid after HPLC purification. Yield: 22%, (4 mg). MS (ESI, M+H⁺)=496.6

### (S)-Phosphoric acid mono-(2-amino-2-[4-(2-biphenyl-4-yl-ethoxy)-3-chloro-phenylcarbamoyl]-propyl}ester

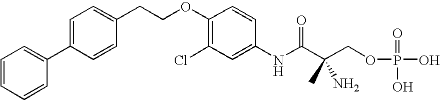

The compound was obtained as a white solid after HPLC purification. Yield: 30%, (70 mg). MS (ESI, M+H⁺)=504.9

### (S)-Phosphoric acid mono-(2-amino-2-[4-(2-biphenyl-4-yl-ethoxy)-3-methyl-phenylcarbamoyl]-propyl}ester

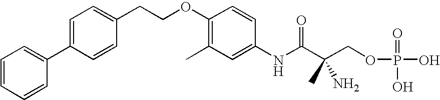

The compound was obtained as a white solid after HPLC purification. Yield: 10%, (28 mg). MS (ESI, M+H⁺)=484.2

### (S)-2-(4-(4-Phenylphenethyloxy)-3-(methylformyl)phenylcarbamoyl)-2-aminopropyl dihydrogen phosphate

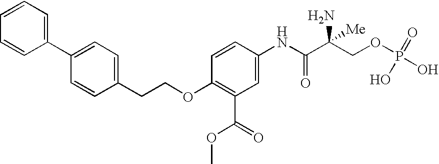

The product was obtained as a white solid in 72% (10.0 mg) yield over two steps. MS (ESI, M+H⁺)=529.1.

### (S)-2-(4-(4-Phenylphenethyloxy)-3-(formyl)phenylcarbamoyl)-2-aminopropyl dihydrogen phosphate

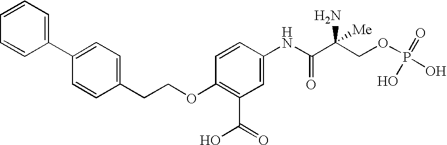

The product was obtained as a white solid in 90% (6.0 mg) yield over two steps. MS (ESI, M+H⁺)=515.0

### (S)-2-(4-(4-Phenylphenethyloxy)-3-(carbamoyl)phenylcarbamoyl)-2-aminopropyl dihydrogen phosphate

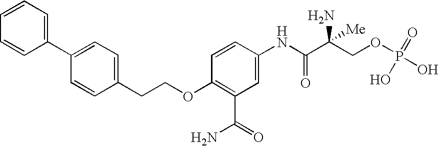

The product was obtained as a white solid in 20% (1.0 mg) yield over four steps. MS (ESI, M+H⁺)=514.6

### (S)-2-(4-(4-Phenylphenethyloxy)-3-(methylcarbamoyl)phenylcarbamoyl)-2-aminopropyl dihydrogen phosphate

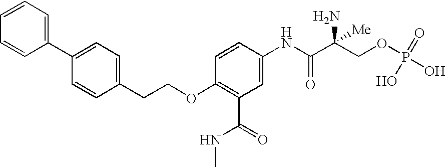

The product was obtained as a white solid in 25% (1.0 mg) yield over four steps. MS (ESI, M+H⁺)=528.6

### (S)-2-(4-(4-Phenylphenethyloxy)-3-(trifluoromethyl)phenylcarbamoyl)-2-aminopropyl dihydrogen phosphate

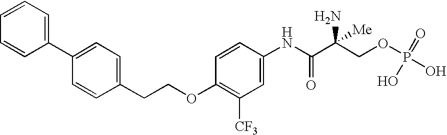

The product was obtained as a white solid in 70% (65.0 mg) yield over four steps. MS (ESI, M+H⁺)=539.7

### (S)-2-(4-(4-Phenylphenethyloxy)-3-bromophenylcarbamoyl)-2-aminopropyl dihydrogen phosphate

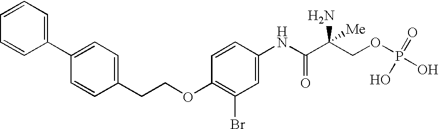

The product was obtained as a white solid in 69% (65.0 mg) yield over four steps. MS (ESI, M+H⁺)=548.9 and 550.9

### (S)-2-(4-(4-(4-Ethylphenyl)phenethyloxy)phenylcarbamoyl)-2-aminopropyl dihydrogen phosphate

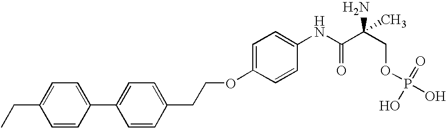

This compound was synthesized from tert-butyl (S)-2-(4-(4-(4-ethylphenyl)phenethyloxy)phenylcarbamoyl)-1-hydroxypropan-2-ylcarbamate (65 mg) to yield 21 mg solid product over two steps. MS (ESI, M+H⁺)=499; ¹H NMR (400 MHz, DMSO-d₆) δ 9.98 (br s, 1H), 7.54 (m, 6H), 7.37 (d, 2H), 7.26 (d, 2H), 6.92 (d, 2H), 4.21 (m, 1H), 4.17 (t, 2H), 4.1 (m, 1H), 3.75 (s, 3H), 3.04 (t, 2H), 2.58 (q, 2H), 1.45 (s, 3H), 1.17 (t, 3H).

### (S)-2-(4-(4-(4-Trifluoromethylphenyl)phenethyloxy)phenylcarbamoyl)-2-aminopropyl dihydrogen phosphate

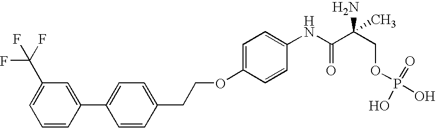

This compound was synthesized from tert-butyl (S)-2-(4-(4-(4-trifluoromethylphenyl)phenethyloxy)phenylcarbamoyl)-1-hydroxypropan-2-ylcarbamate (70 mg) to yield 27 mg solid product over two steps. MS (ESI, M+H⁺)=539; ¹H NMR (400 MHz, DMSO-d₆) δ 9.98 (br s, 1H), 7.95 (m, 2H), 7.69 (d, 4H), 7.5 (d, 2H), 7.4 (d, 2H), 6.9 (d, 2H), 4.21+4.19 (overlapping signals, 3H), 4.05 (m, 1H), 3.06 (t, 2H), 1.45 (s, 3H).

### (S)-2-(4-(4-(4-Ethoxyphenyl)phenethyloxy)phenylcarbamoyl)-2-aminopropyl dihydrogen phosphate

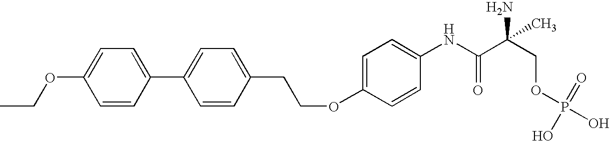

MS (ESI, M+H⁺)=515; ¹H NMR (400 MHz, DMSO-d₆) δ 9.95 (br s, 1H), 7.57 (m, 2H), 7.50 (m, 2H), 7.30 (m, 2H), 6.97 (d, 1H), 6.91 (t, 2H), 4.2-4.0 (m, 2H), 4.10 (t, 2H), 3.1 (m, 2H), 3.0 (m, 2H), 1.45 (s, 3H), 1.32 (t, 3H).

### (S)-2-(4-(4-(4-Chlorophenyl)phenethyloxy)phenylcarbamoyl)-2-aminopropyl dihydrogen phosphate

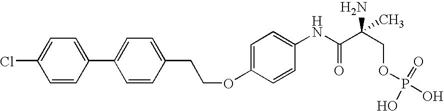

MS (ESI, M+H⁺)=505.7; ¹H NMR (400 MHz, DMSO-d₆) δ 9.95 (s, 1H), 7.69 (m, 2H), 7.61 (s, 1H), 7.49 (m, 3H), 7.37 (t, 1H), 7.33 (m, 1H), 6.94 (d, 2H), 4.3-4.0 (m overlapping signals, 4H), 3.08 (t, 2H), 3.00 (m, 2H), 1.45 (s, 3H).

### (S)-2-(4-(2-(4-Phenyl-3-fluorophenyl)propoxy)phenylcarbamoyl)-2-aminopropyl dihydrogen phosphate

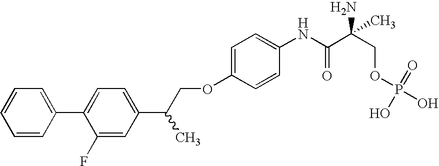

This compound was synthesized from tert-butyl (S)-2-(4-(2-(4-phenyl-3-fluorophenyl)propoxy)phenylcarbamoyl)-1-hydroxypropan-2-ylcarbamate (135 mg) to yield 72 mg solid product over two steps. MS (ESI, M+H⁺)=503; ¹H NMR (400 MHz, DMSO-d₆) δ 9.98 (br s, 1H), 8.6 (br s, 2H), 7.54-7.26 (m, 10H), 6.92 (d, 2H), 4.28 (t, 1H), 4.1-3.9 (m, 3H), 4.1 (m, 1H), 3.28 (m, 2H), 1.49 (s, 3H), 1.35 (d, 3H).

### (S)-2-(4-(4-(Thiophen-3-yl)phenethyloxy)phenylcarbamoyl)-2-aminopropyl dihydrogen phosphate

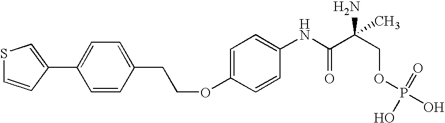

The starting material, 2-(4-(thiophen-3-yl)phenyl)ethanol, was synthesized as follows: In a sealed vessel was combined 2-(4-bromophenyl)ethanol (70 μL), 4,4,5,5-tetramethyl-2-(thiophen-3-yl)-1,3,2-dioxaborolane (126 mg), K₂CO₃ (207 mg), catalytic Pd(PPh₃)₄, 4.5 mL THF, and 0.5 mL H₂O. The vessel was heated in an oil bath at 60° C. overnight. The reaction mixture was diluted with water and DCM. The organic layer was concentrated to yield 2-(4-(thiophen-3-yl)phenyl)ethanol (80 mg) as a solid white product. 80 mg tert-butyl (S)-2-(4-(4-(thiophen-3-yl)phenethyloxy)phenylcarbamoyl)-1-hydroxypropan-2-ylcarbamate was synthesized following the general procedure employing 2-(4-(thiophen-3-yl)phenyl)ethanol (200 mg), N-(Boc)-α-methylserine (175 mg), HATU (375 mg), and DIPEA (430 uL). MS (ESI, M+Na⁺)=519. 2.6 mg of the phosphate was then synthesized from the carbamate (40 mg) as a solid white solid. MS (ESI, M+H⁺)=477; ¹H NMR (400 MHz, DMSO-d₆) δ 9.96 (br s, 1H), 7.81 (m, 1H), 7.65 (m, 3H), 7.5 (m, 3H), 7.3 (m, 2H), 6.9 (m, 2H), 4.28 (m, 1H), 4.17 (m, 2H), 4.06 (m, 1H), 3.04 (t, 2H), 1.48 (s, 3H).

### (S)-2-(4-(4-(Thiophen-2-yl)phenethyloxy)phenylcarbamoyl)-2-aminopropyl dihydrogen phosphate

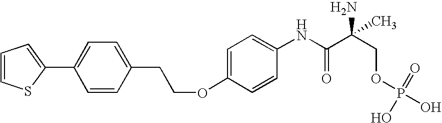

¹H NMR (400 MHz, DMSO-d₆) δ 9.98 (br s, 1H), 8.64 (br s, 3H), 7.84 (s, 1H), 7.65 (m, 3H), 7.52 (m, 3H), 7.36 (d, 2H), 6.9 (d, 2H), 4.21 (overlapping signals, 3H), 4.17 (m, 1H), 3.04 (t, 2H), 2.58 (q, 2H), 1.45 (s, 3H).

### (S)-2-(4-(3-Phenylphenethyloxy)phenylcarbamoyl)-2-aminopropyl dihydrogen phosphate

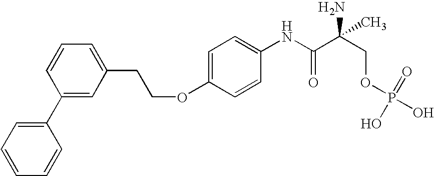

¹H NMR (400 MHz, DMSO-d₆) δ 9.9 (s, 1H), 7.66 (d, 2H), 7.61 (s, 1H), 7.55-7.30 (m, 4H), 6.94 (d, 2H), 4.25 (t, 2H), 4.2 (m, 1H), 4.05 (m, 1H), 3.08 (t, 2H), 1.45 (s, 3H).

### (S)-2-(4-(4-(Pyridin-4-yl)phenethyloxy)phenylcarbamoyl)-2-aminopropyl dihydrogen phosphate

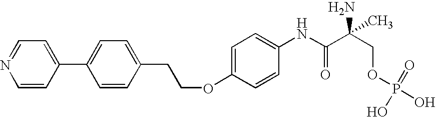

¹H NMR (400 MHz, D₂O+CD₃OD) δ 8.7 (m), 8.2 (m), 7.84 (d, 2H), 7.55 (d, 2H), 7.4 (d, 2H), 6.9 (d, 2H), 4.30 (t, 2H), 4.05 (m, 1H), 3.92 (m, 1H), 3.15 (t, 2H), 1.42 (s, 3H).

### (S)-2-(4-(4-(Pyridin-3-yl)phenethyloxy)phenylcarbamoyl)-2-aminopropyl dihydrogen phosphate

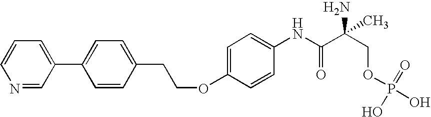

¹H NMR (400 MHz, DMSO-d₆) δ 9.95 (s, 1H), 8.95 (s, 1H), 8.65 (br s, 1H), 8.28 (d, 2H), 7.75 (m, 2H), 7.60 (m, 1H), 7.5 (t, 3H), 6.95 (d, 2H), 4.2 (m, 3H), 3.95 (m, 1H), 3.10 (m, 2H), 1.45 (s, 3H).

### Example 11

### Synthesis of Phenylimidazole/Phenyloxazole Analogs

General Method for Synthesis of Phenylimidazole/Phenyloxazole

The general approach for synthesis of various phenylazole compounds 5 is described in Scheme 18. Reaction of desired alcohol with substituted 4-fluoroacetophenone 1 at 60-70° C. afforded the ether-acetophenone intermediate 2. The ether-acetophenone 2 was then converted to the bromo-acetophenone using CuBr₂. Reaction of the bromo-acetophenone with Boc-a-MeSer afforded the desired ester 3 which was then converted to the azole precursor 4 using AcONH₄. Deprotection of the Boc group provided the TFA salt of the final compound 5 in good yield. The final compounds were then isolated either as TFA or HCl salts.

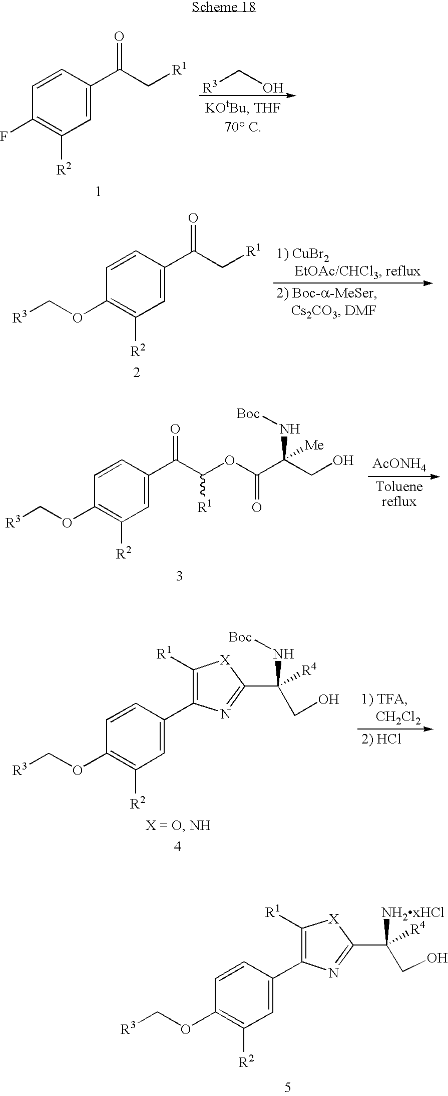

### Synthesis of biphenyl-3-ylmethanol

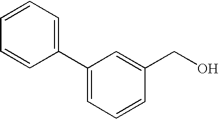

To a mixture of (3-iodophenyl)methanol (0.5 mL, 1.0 equiv), phenyl boronic acid (0.72 g, 1.5 equiv), Pd(OAc)₂ (88 mg, 0.1 equiv), PPh₃ (210 mg, 0.2 equiv), and Cs₂CO₃ (1.28 g, 1.0 equiv) in DMF(20 mL) was heated at 70° C. overnight. The reaction was then diluted with EtOAc (25 mL) and washed with H₂O (2×25 mL) then the solvent removed in vacuo. The crude product was purified by silica gel column chromatography using Combi-Flash system (Hex:EtOAc) as needed. The product was obtained a white solid in 73% (0.53 g) yield. TLC (1:2 EtOAc:Hex), R_{f}=0.4; ¹H NMR (400 MHz, CDC₁₃) δ 7.58-7.63 (m, 3H), 7.51-7.56 (m, 1H), 7.42-7.48 (m, 3H), 7.33-7.39 (m, 2H), 4.57 (s, 2H).

General Protocol for Synthesis of Substituted Acetophenones (Williamson Ether Synthesis)

To a solution of the desired alcohol (1.0 equiv) in dry THF under nitrogen atmosphere was added KO^{t}Bu (either 1.0 M solution in THF or solid, 1.1 equiv). The reaction mixture was heated at 60-70° C. for 15 minutes, then substituted 4-flouroacetophenone (1.0 equiv) was added. The reaction was then stirred for 30 minutes before cooling to room temperature and quenching with water. The mixture was then diluted with EtOAc and washed with H₂O (2×), saturated NaCl (1×), dried with MgSO₄. The organic layer was then concentrated under reduced pressure. The product did not require any further purification.

### 1-(4-(Octyloxy)-3-(trifluoromethyl)phenyl)ethanone

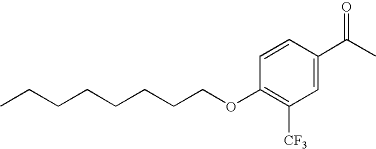

The product was purified by silica gel column chromatography using the Combi-Flash system (Hex:EtOAc) as white solid in 60% (1.20 g). TLC (1:5 EtOAc:Hex), R_{f}=0.4; ¹H NMR (400 MHz, CDC₁₃) δ 8.18 (d, 1H, J=2.0 Hz), 8.10 (dd, 1H, J=8.8 Hz, J=2.3 Hz), 7.02 (d, 1H, J=8.8 Hz), 4.12 (t, 2H, J=6.4 Hz), 2.58 (m, 3H), 1.80-1.89 (m, 2H), 1.42-1.54 (m, 2H), 1.22-1.40 (m, 8H), 0.89 (t, 3H, J=6.7 Hz).

### 1-(4-(4-Phenylbenzyloxy)-3-(trifluoromethyl)phenyl)ethanone

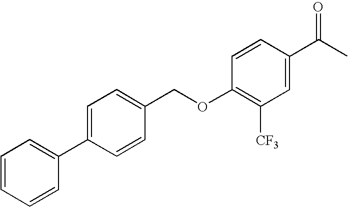

The product was purified by silica gel columnn chromatography using the Combi-Flash system (Hex:EtOAc) as white solid in 92% (5.88 g). Proton NMR and LC analyses confirmed the desired product with purity greater than 95%.

TLC (1:3 EtOAc:Hex), R_{f}=0.3; ¹H NMR (400 MHz, CDC₁₃) δ 8.23 (d, 1H, J=2.3 Hz), 8.12 (dd, 1H, J=8.6 Hz, J=2.3 Hz), 7.57-7.65 (m, 4H), 7.42-7.53 (m, 4H), 7.33-7.39 (m, 1H), 7.12 (d, 1H, J=8.6 Hz), 5.33 (s, 2H), 2.59 (s, 3H).

### 1-(4-(Biphenyl-3-ylmethoxy)-3-(trifluoromethyl)phenyl)ethanone

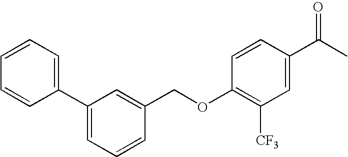

The product was purified by silica gel column chromatography using the Combi-Flash system (Hex:EtOAc) as white solid in 51% (0.54 g). Proton NMR and LC analyses confirmed the desired product.

TLC (1:2 EtOAc:Hex), R_{f}=0.5; ¹H NMR (400 MHz, CDC₁₃) δ 8.23 (d, 1H, J=2.3 Hz), 8.12 (dd, 1H, J=8.6 Hz, J=2.3 Hz), 7.68 (br s, 1H), 7.55-7.62 (m, 3H), 7.34-7.50 (m, 5H), 7.10 (d, 1H, J=8.6 Hz), 5.34 (s, 2H), 2.58 (s, 3H).

### 1-(4-(Biphenyl-4-ylmethoxy)-3-(trifluoromethyl)phenyl)propan-1-one

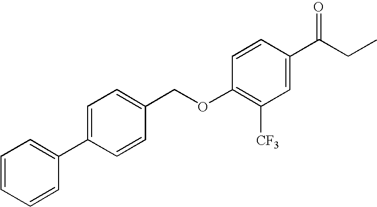

The product was purified by silica gel column chromatography using the Combi-Flash system (Hex:EtOAc) as white solid in 83%. TLC (1:3 EtOAc:Hex), R_{f}=0.4; ¹H NMR (400 MHz, CDC₁₃) δ 8.24 (d, 1H, J=2.0 Hz), 8.12 (dd, 1H, J=8.8 Hz, J=2.4 Hz), 7.56-7.65 (m, 4H), 7.50 (d, 2H, J=8.4 Hz), 7.45 (t, 2H, J=8.2 Hz), 7.36 (t, 1H, J=8.0 Hz), 7.11 (d, 1H, J=8.8 Hz), 5.31 (s, 2H), 2.97 (q, 2H, J=7.2 Hz), 1.23 (t, 3H, J=7.2 Hz).

### 1-(4-(Biphenyl-4-ylmethoxy)phenyl)ethanone

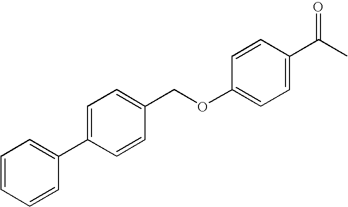

The product was purified by silica gel column chromatography using the Combi-Flash system (Hex:EtOAc) as white solid in 84%. TLC (1:3 EtOAc:Hex), R_{f}=0.3; ¹H NMR (400 MHz, CDC₁₃) δ 7.95 (d, 2H, J=8.8 Hz), 7.57-7.65 (m, 5H), 7.50 (d, 2H, J=8.0 Hz), 7.45 (t, 2H, J=8.0 Hz), 7.36 (t, 1H, J=8.0 Hz), 7.03 (d, 1H, J=8.8 Hz), 5.17 (s, 2H), 2.56 (s, 3H).

General Protocol for Synthesis of Substituted Phenylimidazole/Phenyloxazole

To a solution of the substituted acetophenone (1.0 equiv) in EtOAc/CHC₁₃ (1:1) under nitrogen atmosphere was added CuBr₂ (3.0 equiv). The reaction mixture was heated at reflux for 2-5 hours. The reaction was then diluted with EtOAc and washed with H₂O (2×) and saturated NaCl (1×). The organic layer was dried over anhydrous MgSO₄ then the solvent removed in vacuo. The crude product was either carried forward as is or was purified by silica gel column chromatography using the Combi-Flash system (Hex:EtOAc).

To a mixture of the desired bromo-acetophenone (from last step, 1.0 equiv), Boc-α-MeSer (1.0 equiv), and Cs₂CO₃ (0.6 equiv) was stirred in DMF for 1-2 hours. The reaction mixture was diluted with EtOAc and washed with H₂O (2×), and saturated NaCi (1×) to remove access DMF and CsBr salt. The organic layer was dried over anhydrous MgSO₄ and the solvent removed in vacuo. TLC generally showed a spot to spot conversion of the starting material to product.

To the obtained ester was then added excess ammonium acetate (10 equiv), and the mixture was suspended in toluene and refluxed for 3-6 hours under Dean-Stark conditions. The mixture was diluted with EtOAc and washed with H₂0 (2×), and saturated NaCl (1×). The solvent removed in vacuo. The product was purified by silica gel column chromatography using the Combi-Flash system (Hex:EtOAc).

TLC and LC analyses showed complete consumption of the starting material, formation of the desired product with purity of 60%. The TLC and LC analyses also showed two other compounds which were not isolated. The crude product was carried forward as is. TLC (1:3 EtOAc:Hex), R_{f}=0.4.

### (R)-tert-Butyl 1-hydroxy-2-(5-(4-(octyloxy)-3-(trifluoromethyl)phenyl)-1H-imidazol-2-yl)propan-2-ylcarbamate

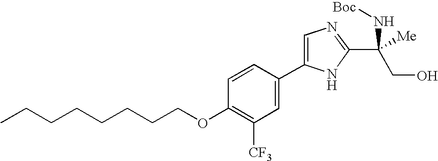

TLC and LC analyses showed complete consumption of the acetophenone, formation of the bromo-acetophenone with purity of 60%. The TLC and LC analyses also showed two other compounds. The crude product was carried forward as is. TLC (1:3 EtOAc:Hex), R_{f}=0.6.

The final product was purified by silica gel column chromatography using the Combi-Flash system (Hex:EtOAc) as yellow solid in 33% (0.65 g). TLC (2:1 EtOAc:Hex), R_{f}=0.5; MS (ESI, M+H⁺)=514.4.

### tert-Butyl (R)-2-(5-(4-(4-phenylbenzyloxy)-3-(trifluoromethyl)phenyl)-1H-imidazol-2-yl)-1-hydroxypropan-2-ylcarbamate

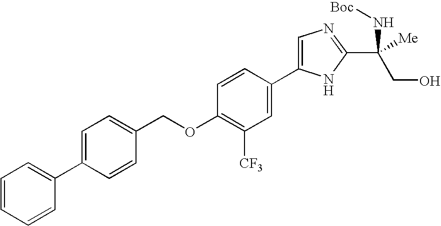 TLC and LC analyses showed complete consumption of the acetophenone, formation of the bromo-acetophenone with purity of 60%. The TLC and LC analyses also showed two other compounds. The crude product was carried forward as is. TLC (1:3 EtOAc:Hex), R_{f}=0.4.

The final product was purified by silica gel column chromatography using the Combi-Flash system (Hex:EtOAc) as yellow solid in 45% (1.37 g). Proton NMR and LC-MS analyses confirmed the desired product with purity greater than 90%.

TLC (1:1 EtOAc:Hex), R_{f}=0.3; MS (ESI, M+H⁺)=568.3; ¹H NMR (400 MHz, CDC₁₃) δ 7.90 (br s, 1H), 7.80 (br d, 1H, J=8.8 Hz), 7.58-7.64 (m, 4H), 7.52 (d, 2H, J=8.6 Hz), 7.41-7.47 (m, 2H), 7.33-7.38 (m, 1H), 7.16 (s, 1H), 7.07 (d, 1H, J=8.8 Hz), 5.72 (br s, 1H), 5.26 (s, 2H), 4.31 (d, 1H, J=11.2 Hz), 3.65 (d, 1H, J=11.2 Hz), 1.66 (s, 3H), 1.43 (s, 9H).

General Protocol for Deprotection of the Amino Group

To a solution of the Boc-protected precursor (1.0 equiv) in CH₂C₁₂ was added TFA (25% by volume). The reaction mixture was stirred at room temperature for 1-2 hours then evaporated to dryness under reduced pressure to afford the final compound. The final product was purified by reverse-phase preparative HPLC, as needed, as TFA salt.

In order to generate the desired HCl salt, the product was then purified by silica gel column chromatography using the Combi-Flash system (CH₂C₁₂:IPA). The free amine was then dissolved in EtOAc/EtOH (1:1) and cooled to 0° C. To the solution was added conc. HCl (1.1 equiv) drop-wise. The reaction was stirred at 0° C. for 15 minute then the solvent was removed in vacuo. The product was crashed from MeCN and collected by filtration.

### (R)-2-Amino-2-(5-(4-(octyloxy)-3-(trifluoromethyl)phenyl)-1H-imidazol-2-yl)propan-1-ol

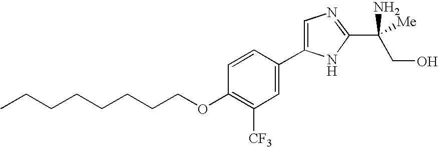

The final product was purified by reverse phase preparative HPLC, then lyophilized to dryness to obtain TFA salt of the product. The product was obtained as a white solid in 39% (40 mg) yield. MS (ESI, M+H⁺)=414.2; ¹H NMR (400 MHz, DMSO-d₆) δ 8.41 (br s, 3H), 8.03 (d, 1H, J=1.5 Hz), 7.96 (dd, 1H, J=8.8 Hz, J=2.0 Hz), 7.72 (br s, 1H), 7.24 (d, 1H, J=8.4), 5.70 (br s, 1H), 4.09 (t, 2H, J=6.2 Hz), 3.75 (d, 1H, J=11.2 Hz), 3.64 (d, 1H, J=11.2 Hz), 1.65-1.76 (m, 2H), 1.54 (s, 3H), 1.36-1.48 (m, 2H), 1.18-1.36 (m, 8H), 0.84 (t, 3H, J=6.8 Hz).

### (R)-2-Amino-2-(5-(4-(4-phenylbenzyloxy)-3-(trifluoromethyl)phenyl)-1H-imidazol-2-yl)propan-1-ol

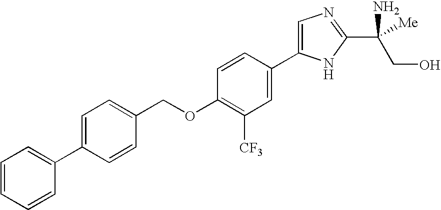

The final product was purified by reverse phase preparative HPLC, then lyophilized to dryness to obtain TFA salt of the product. The product was obtained as a white solid in 61% (375 mg) yield. MS (ESI, M+H⁺)=468.3; ¹H NMR (400 MHz, DMSO-d₆) δ 8.42 (br s, 3H), 8.09 (d, 1H, J=1.5 Hz), 7.99 (dd, 1H, J=8.8 Hz, J=2.4 Hz), 7.76 (br s, 1H), 7.64-7.73 (m, 4H), 7.54 (d, 2H, J=8.2 Hz), 7.33-7.49 (m, 4H), 5.32 (s, 2H), 3.74 (d, 1H, J=11.0 Hz), 3.63 (d, 1H, J=11.0 Hz), 1.53 (s, 3H).

### (R)-2-Amino-2-(5-(4-(biphenyl-3-ylmethoxy)-3-(trifluoromethyl)phenyl)-1H-imidazol-2-yl)propan-1-ol

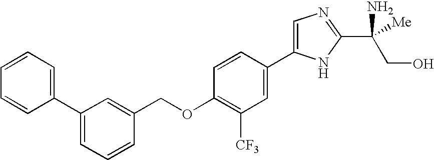

The final product was purified by reverse phase preparative HPLC, then lyophilized to dryness to obtain TFA salt of the product. The product was obtained as a white solid in 77% (78 mg) yield. MS (ESI, M+H⁺)=468.2; ¹H NMR (400 MHz, DMSO-d₆) δ 8.42 (br s, 3H), 8.09 (d, 1H, J=2.0 Hz), 8.01 (dd, 1H, J=8.4 Hz, J=2.0 Hz), 7.75 (d, 2H, J=7.8 Hz), 7.60-7.67 (m, 3H), 7.34-7.53 (m, 6H), 5.35 (s, 2H), 3.74 (d, 1H, J=11.6 Hz), 3.64 (d, 1H, J=11.6 Hz), 1.54 (s, 3H).

### (R)-2-Amino-2-(5-(4-(biphenyl-4-ylmethoxy)-3-(trifluoromethyl)phenyl)-1H-imidazol-2-yl)ethanol

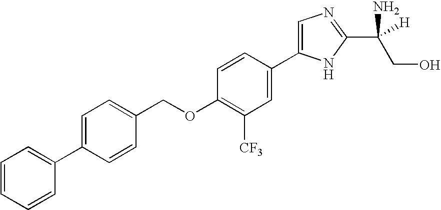

The final product was purified by reverse phase preparative HPLC, then lyophilized to dryness to obtain TFA salt of the product. The product was obtained as a white solid in 85% (35 mg) yield. MS (ESI, M+H⁺)=454.3; ¹H NMR (400 MHz, DMSO-d₆) δ 8.47 (br s, 3H), 8.07 (d, 1H, J=1.6 Hz), 8.00 (dd, 1H, J=8.8 Hz, J=2.4 Hz), 7.66-7.76 (m, 5H), 7.55 (d, 2H, J=8.4 Hz), 7.35-7.50 (m, 4H), 5.34 (s, 2H), 4.38 (br s, 1H), 3.76-3.90 (m, 2H).

### (R)-2-Amino-2-(5-(4-(biphenyl-4-ylmethoxy)-3-(trifluoromethyl)phenyl)-4-methyl-1H-imidazol-2-yl)propan-1-ol

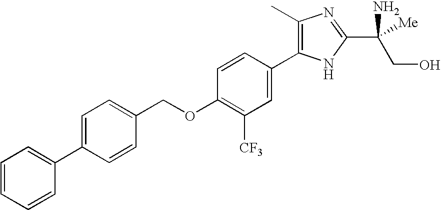

The reaction to synthesize the Boc protected precursor to imidazole product afforded a 1:1 ratio of the desired both Boc protected precursors imidazole and oxazole. The final product was purified by reverse phase preparative HPLC, then lyophilized to dryness to obtain TFA salt of the product. MS (ESI, M+H⁺)=482.4; ¹H NMR (400 MHz, DMSO-d₆) δ 8.37 (br s, 3H), 7.96 (br s, 1H), 7.82 (dd, 1H, J=8.4 Hz, J=2.0 Hz), 7.74 (d, 2H, J=8.4 Hz), 7.70 (d, 2H, J=7.2 Hz), 7.57 (d, 2H, J=8.4 Hz), 7.47 (t, J=7.6 Hz, 2H), 7.43 (d, J=9.2 Hz, 1H), 7.37 (t, 1H, J=7.6 Hz), 5.68 (br s, 1H), 5.34 (s, 2H), 3.76 (d, 1H, J=11.6 Hz), 3.66 (d, 1H, J=11.6 Hz), 2.39 (s, 3H), 1.54 (s, 3H).

### (R)-2-Amino-2-(5-(4-(biphenyl-4-ylmethoxy)phenyl)-1H-imidazol-2-yl)propan-1-ol

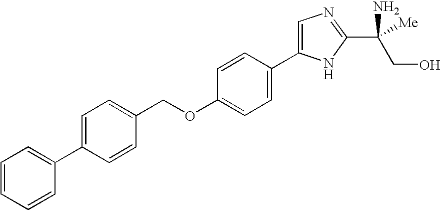

The final product was purified by reverse phase preparative HPLC, then lyophilized to dryness to obtain TFA salt of the product. MS (ESI, M+H⁺)=400.4; ¹H NMR (400 MHz, DMSO-d₆) δ 8.39 (br s, 3H), 7.73 (br s, 1H), 7.71-7.67 (m, 6H), 7.56 (d, 2H, J=8.4 Hz), 7.47 (t, 2H, J=7.6 Hz), 7.37 (d, 1H, J=8.6 Hz), 7.07 (d,2H, J=8.8 Hz), 5.68 (br s, 1H), 5.16 (s, 2H), 3.77 (d, 1H, J=1.0 Hz), 3.63 (d, 1H, J=11.2 Hz), 1.56 (s, 3H).

### (S)-2-Amino-2-(4-(4-(biphenyl-4-ylmethoxy)-3-(trifluoromethyl)phenyl)-5-methyloxazol-2-yl)propan-1-ol

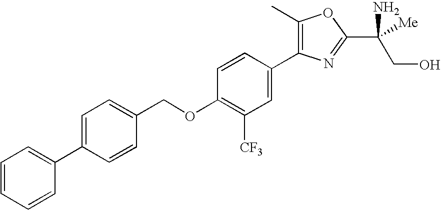

The reaction to synthesize the Boc protected precursor to imidazole analog afforded a 1:1 ratio of the Boc protected both imidazole and oxazole. The final product was purified by reverse phase preparative HPLC, then lyophilized to dryness to obtain TFA salt of the product. MS (ESI, M+H⁺)=483.4; ¹H NMR (400 MHz, DMSO-d₆) δ 8.70 (br s, 3H), 7.96 (d, 1H, J=2.0 Hz, 1H), 7.92 (dd, 1H, J=8.4 Hz, J=2.0 Hz), 7.74 (d, 2H, J=8.4 Hz), 7.70 (d, 2H, J=7.2 Hz), 7.57 (d, 2H, J=8.4 Hz), 7.50-7.45 (m, 3H), 7.37 (t, 1H, J=7.6 Hz), 5.83 (br s, 1H), 5.38 (s, 2H), 3.86 (dd, 1H, J=11.0 Hz, J=4.4 Hz), 3.71 (dd, 1H, J=11.2 Hz, J=4.4), 2.57 (s, 3H), 1.56 (s, 3H).

Synthesis of Phenylthiazoles

The syntheses of two isomers of phenylthiazole are described in Scheme 19 and 20. In Scheme 19, Boc-α-MeSer 1 was converted to protected oxazolidine-4-carboxylic acid 2 in three simple steps. Oxazolidine-4-carboxylic acid 2 was then converted to an amide subsequently to a thioamide 3 in good yields. Reaction of thioamide 3 with bromo-acetophenone 4 afforded the protected thiazole precursor 5. The thiazole precursor 5 was then deportected to afford the desired the final compound 6.

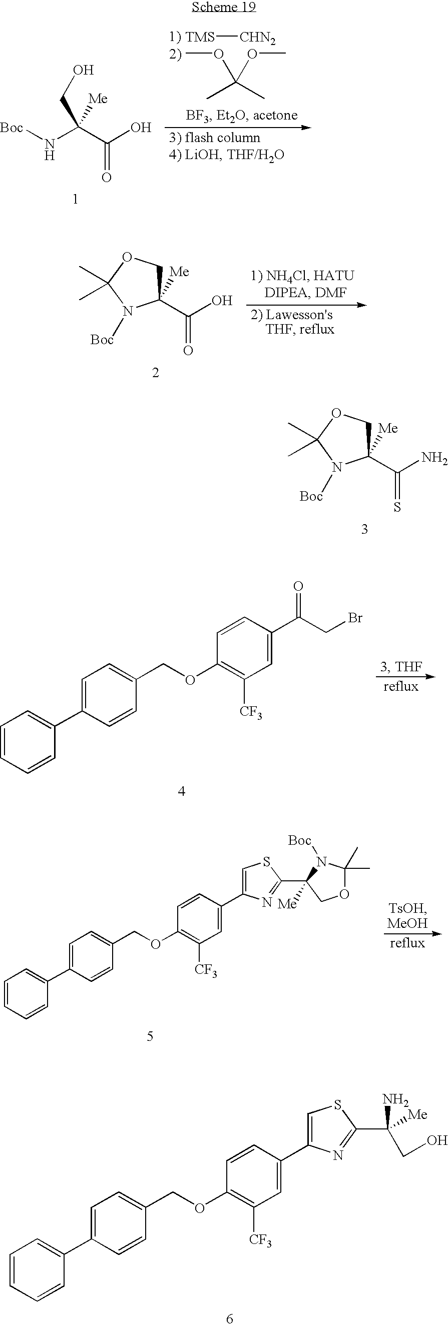

In Scheme 20, Reaction of the ether-acetophenone 1 with CuBr₂ afforded bromo-acetophenone which upon reaction with NaN₃ provided azido-acetophenone 2. Hydrogenation of the azido-acetophenone 2 followed by coupling with protected oxazolidine-4-carboxylic acid 4 afforded oxazolidine-amide 5. Oxazolidine-amide 5 was then under Lawesson's reagent conditions was then converted to protected- oxazolidine-thioamide 6 in good yields. De-protection of orthogonally protected oxazolidine-thioamide 6 afford the desired the final compound 7 in excellent yield.

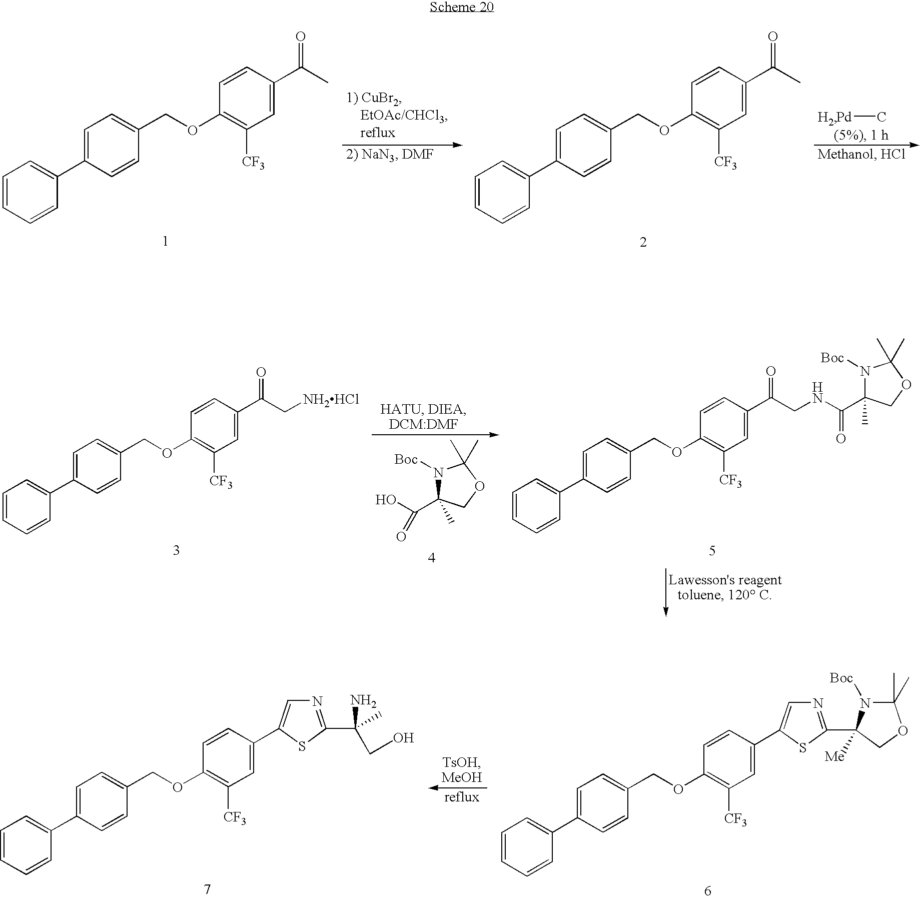

### (S)-tert-butyl 4-carbamothioyl-2,2,4-trimethyloxazolidine-3-carboxylate

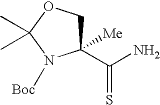

To a mixture of the (S)-3-(tert-butoxycarbonyl)-2,2,4-trimethyloxazolidine-4-carboxylic acid (0.50 g, 1.0 equiv), NH₄Cl (1.03 g, 10.0 equiv), and HATU (1.10 g, 1.5 equiv) in DMF (10 mL) under nitrogen atmosphere was added DIPEA (2.50 mL, 10.0 equiv). The reaction mixture was stirred overnight. The reaction was then diluted with EtOAc (50 mL), washed with 10% NH₄Cl (2×50 mL) and saturated NaCl (1×50 mL). The organic layer was dried over anhydrous MgSO₄ then the solvent removed in vacuo. The crude product was carried forward as is. TLC (EtOAc), R_{f}=0.2.

To a mixture of the desired amide (from last step, 1.0 equiv) and Lawesson's reagent (3.9 g, 5.0 equiv) was added dry THF (20 mL) then refluxed overnight. The solvent removed in vacuo. TLC showed a spot to spot conversion of the starting material to product. The product was purified by silica gel column chromatography using the Combi-Flash system (Hex:EtOAc). The product was obtained as a white solid in 76% (400 mg) yield.

TLC (EtOAc), R_{f}=0.5; ¹H NMR (400 MHz, DMSO-d₆) δ 4.15 (br s, 2H), 3.74-3.86 (m, 2H), 1.64 (s, 3H), 1.57 (s, 3H), 1.46 (s, 3H), 1.35 (s, 9H).

### (S)-tert-butyl 4-(4-(4-(biphenyl-4-ylmethoxy)-3-(trifluoromethyl)phenyl)thiazol-2-yl)-2,2,4-trimethyloxazolidine-3-carboxylate

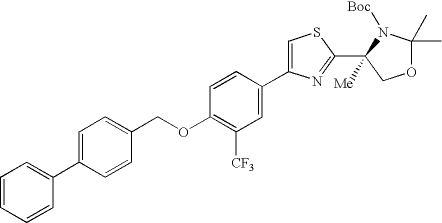

A mixture of the (S)-tert-butyl 4-carbamothioyl-2,2,4-trimethyloxazolidine-3-carboxylate (0.40 g, 1.0 equiv) and bromo-acetophenone (0.66 g, 1.0 equiv) were dissolved in dry THF (10 mL) under nitrogen atmosphere then refluxed overnight. The solvent was then evaporated to dryness in vacuo. The product was purified by silica gel column chromatography using the Combi-Flash system (Hex:EtOAc) to obtained a thick colourless oil in 60% (0.55 g) yield. TLC (3: 1, Hex/EtOAc), R_{f}=0.6.

### (S)-2-amino-2-(4-(4-(biphenyl-4-ylmethoxy)-3-(trifluoromethyl)phenyl)thiazol-2-yl)propan-1-ol

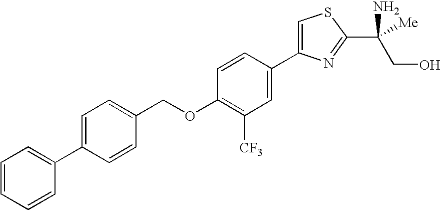

The product was purified by silica gel column chromatography using the Combi-Flash system (CH₂C₁₂:IPA) to afford as a white solid in 99% (430 mg) yield. MS (ESI, M+H⁺)=485.4; ¹H NMR (400 MHz, DMSO-d₆) δ 8.66 (br s, 3H), 8.34 (d, 1H, J=2.0 Hz), 8.30 (s, 1H), 8.25 (dd, 1H, J=8.8 Hz, J=1.6 Hz), 7.66-7.75 (m, 4H), 7.44-7.58 (m, 5H), 7.34-7.40 (m, 1H), 5.39 (s, 2H), 3.82 (d, 1H, J=11.6 Hz), 3.75 (d, 1H, J=11.6 Hz), 1.66 (s, 3H).

### 2-Azido-1-(4-(biphenyl-4-ylmethoxy)-3-(trifluoromethyl)phenyl)ethanone

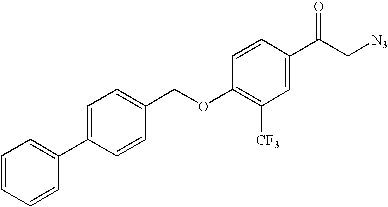

To a solution of the substituted acetophenone (1.65 g, 1.0 equiv) in EtOAc/CHC₁₃ (1.1) under nitrogen atmosphere was added CuBr₂ (3.0 g, 3.0 equiv). The reaction mixture was heated at reflux for 3 hours. The reaction was then diluted with EtOAc and washed with H₂O (2×) and saturated NaCl (1×). The organic layer was dried over anhydrous MgSO₄ then the solvent removed in vacuo. TLC (2:1, Hex/EtOAc), R_{f}=0.5.

To a mixture of the desired bromo-acetophenone (from last step, 1.0 equiv), in DMF (20 mL) was added NaN₃ (0.87 g, 3.0 equiv), then stirred in DMF for 20 minutes. The reaction mixture was diluted with EtOAc (50 mL) and washed with H₂O (2×50). The solvent removed in vacuo and the product was purified by silica gel column chromatography using the Combi-Flash system (Hex:EtOAc) as a white solid in 36% (0.65 g) yield. TLC (2:1, Hex/EtOAc), R_{f}=0.3; ¹H NMR (400 MHz, CDC₁₃) δ 8.17 (d, 1H, J=2.4 Hz), 8.07 (dd, 1H, J=8.8 Hz, J=2.4 Hz), 7.56-7.65 (m, 4H), 7.42-7.52 (m, 4H), 7.38 (t, 1H, J=8.2 Hz), 7.16 (d, 1H, J=8.0 Hz), 5.34 (s, 2H), 4.52 (s, 2H).

### (R)-tert-Butyl 4-(2-(4-(biphenyl-4-ylmethoxy)-3-(trifluoromethyl)phenyl)-2-oxoethyl-carbamoyl)-2,2,4-trimetyloxazolidine-3-carboxylate

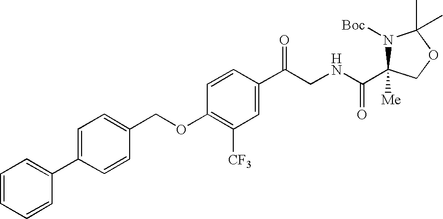

To a solution of the azido-acetophenone (0.99 g, 1.0 equiv) in MeOH (20 mL) was added concentrated HCl (3.0 mL), and 10% Pd/C (99 mg). The reaction mixture was stirred under an atmosphere of H₂ (g) for 2 hours. The reaction was then filtered through a thin layer of Celite then the solvent removed in vacuo. The obtained white solid amino-acetophenone was carried forward as is.

To a solution of protected oxazolidine-4-carboxylic acid (697 mg, 1 equiv), HATU (1.12 g, 1.2 equiv), and DIEA (2.13 mL, 4.5 equiv) in DCM:DMF (5:1, 20 mL) was added amino-acetophenone. The resultant mixture was stirred at RT for 1 h. The reaction was then condensed in vacuo and the residue was purified by chromatography (silica gel, hexane:ethyl acetate, 70:30, v/v) to afford the title compound (860 mg, 57% yield). MS (ESI, M+Na)=649.5; ¹H NMR (400 MHz, CDC₁₃) δ 8.26 (s, 1H), 7.78 (d, 1H, J=8.0 Hz), 7.64 (d, 2H, J=8.0 Hz), 7.60 (d, 2H, J=7.2 Hz), 7.51 (d, 2H, J=8.0 Hz), 7.44 (t, 2H, J=6.8 Hz), 7.35 (t, 1H, J=7.2 Hz), 7.16 (d, 1H, J=9.2 Hz), 5.33 (s, 2H), 4.73 (dd, 2H, J=6.0 Hz, J=4.8 Hz), 4.43 (d, 1H, J=8.8 Hz), 4.18 (d, 1H, J=8.4 Hz), 1.66-1.56 (m, 9H), 1.50 (s, 3H), 1.43 (s, 6H).

### (R)-tert-Butyl-4-(5-(4-(biphenyl-4-ylmethoxy)-3-(trifluoromethyl)phenyl)thiazol-2-yl)-2,2,4-trimethyloxazolidine-3-carboxylate

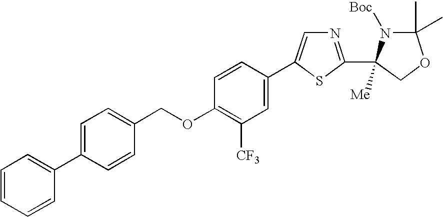

The suspension of protected oxazolidine-amide (120 mg, 1.0 equiv) and Lawesson's Reagent (387 mg, 5 equiv) in toluene (5 mL) was sealed and heated at 120° C. for 1.5 hours. After cooling to RT, the reaction was filterated and the filtrate was condensed and purified by chromatography (silica gel, hexane:ethyl acetate, 4:1, v/v) to afford the title compound (81 mg, yield 64%). MS (ESI, M+H⁺)=625.7; ¹H NMR (400 MHz, CDCl3) δ 7.74 (s, 2H), 7.63-7.58 (m, 5H), 7.52 (d, 2H, J=8.4 Hz), 7.44 (t, 2H, J=8.0 Hz), 7.35 (t, 1H, J=7.2 Hz), 7.09 (d, 1H, J=8.4 Hz), 5.27 (s, 2H), 4.18 (d, 1H, J=8.8 Hz), 4.03 (d, 1H, J=9.6 Hz), 1.90 (s, 3H), 1.80 (s, 3H), 1.67 (s, 3H), 1.50 (s, 3H), 1.27 (s, 6H).

### (S)-2-Amino-2-(5-(4-(biphenyl-4-ylmethoxy)-3-(trifluoromethyl)phenyl)thiazol-2-yl)propan-1-ol

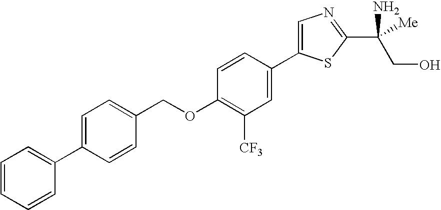

The final product was purified by reverse phase preparative HPLC, then lyophilized to dryness to obtain TFA salt of the product. MS (ESI, M+H⁺)=485.5; ¹H NMR (400 MHz, DMSO-d₆) δ 8.55 (br s, 2H), 8.33 (s, 1H), 7.95 (dd, 1H, J=8.4 Hz, J=2.0 Hz), 7.92 (s, 1H), 7.73 (d, 2H, J=8.4 Hz), 7.70 (d, 2H, J=8.4 Hz), 7.56 (d, 2H, J=8.4 Hz), 7.47 (t, 3H, J=8.0 Hz), 7.37 (t, 1H, J=7.2 Hz), 5.91 (br s, 1H), 5.40 (s, 2H), 3.81 (dd, 1H, J=10.8, Hz, J=4.8 Hz), 3.74 (dd, 1H, J=10.8 Hz, J=4.8 Hz), 1.646 (s, 3H).

General Method for Phosphate Synthesis

The methods are illustrated in Scheme 21 below.

In first approach (Approach A), to a solution of unprotected or Boc-protected amino alcohol (1.0 equiv) in dry CH₂C₁₂ at room temperature was added excess diethyl chlorophosphate (5.0-20.0 equiv) and triethylamine (5.0-30.0 equiv) and the reaction stirred for 12-18 hours. The crude reaction mixture was then loaded onto a silica gel column chromatography, as is, to purify the desired phospho-diester. The phopho-diester intermediate was reacted with excess bromotrimethylsilane (10.0-20.0 equiv) in dry CH₂C₁₂ at room temperature, under an atmosphere of nitrogen; over a period of 6-10 hours afforded the final phosphate which was purified by reverse-phase preparative HPLC.

In second approach (Approach B), to a solution of Boc-protected amino alcohol (1.0 equiv) and 1-H-tetrazole (2.0-6.0 equiv) in dry THF or a s 1:1 mixture of THF/CH₂C₁₂ at room temperature was added di-tert-butyl N,N- diisopropylphosphoramidite (1.0-2.0 equiv) and the reaction was stirred for 2-12 hours. The reaction was monitored by LC-MS and TLC. To the mixture was then added excess H₂O₂ and stirred for 1-6 hours. The reaction was worked up with excess Na₂S₂O₃ or Na₂S₂O₅ to quench excess H₂O₂. The crude was then loaded purified by silica gel column chromatography. The protected phopho-diester intermediate was reacted with excess TFA in dry CH₂C₁₂ (1:3) at room temperature over a period of 1-3 hours to afford the final phosphate. The final product was used as is after evaporation of the solvent and the reagents or purified by reverse-phase preparative HPLC as needed.

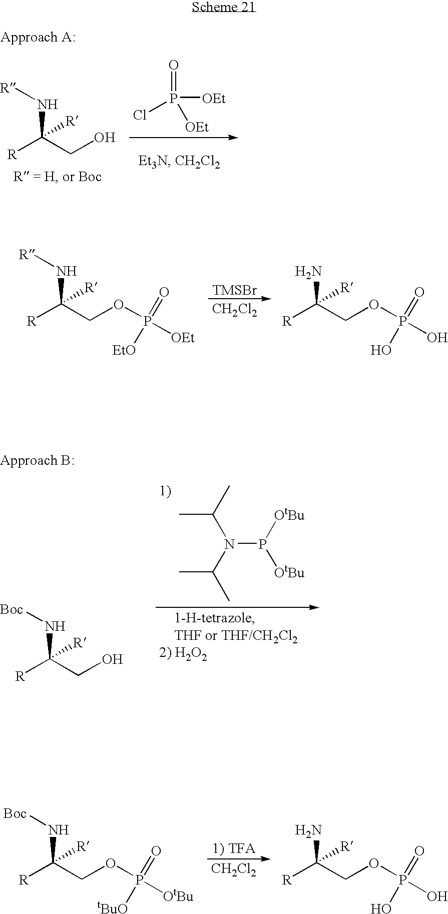

### (R)-2-Amino-2-(5-(4-(octyloxy)-3-(trifluoromethyl)phenyl)-1H-imidazol-2-yl)propyl dihydrogen phosphate

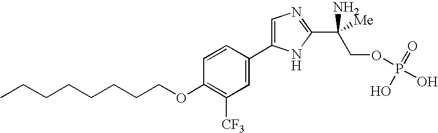

The final product was purified by reverse phase preparative HPLC, then lyophilized to dryness to obtain TFA salt of the product. The product was obtained as a white solid from the alcohol precursor in 21% (25 mg) yield. MS (ESI, M+H⁺)=494.8; ¹H NMR (400 MHz, DMSO-d₆) δ 8.02 (d, 1 H, J=2.4 Hz), 7.96 (dd, 1 H, J=8.6 Hz, J=1.6 Hz), 7.70 (br s, 1H), 7.24 (d, 1H, J=8.8), 5.70 (br s, 1H), 4.12-4.20 (m, 1H), 4.01-4.11 (m, 3H), 1.66-1.76 (m, 2H), 1.59 (s, 3H), 1.36-1.46 (m, 2H), 1.20-1.35 (m, 8H), 0.84 (t, 3H, J=7.2 Hz).

### (R)-2-amino-2-(5-(4-(biphenyl-4-ylmethoxy)-3-(trifluoromethyl)phenyl)-1H-imidazol-2-yl)propyl dihydrogen phosphate

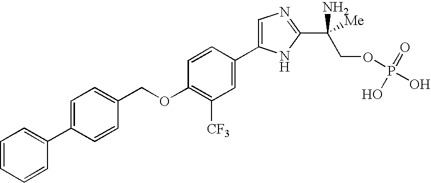

The final product was purified by reverse phase preparative HPLC, then lyophilized to dryness to obtain TFA salt of the product. The product was obtained as a white solid in 24% (70 mg) yield from the alcohol precursor. MS (ESI, M+H⁺)=548.4; ¹H NMR (400 MHz, DMSO-d₆) δ 8.10 (d, 1H, J=2.0 Hz), 8.02 (dd, 1H, J=8.8 Hz, J=1.6 Hz), 7.66-7.78 (m, 5H), 7.55 (d, 2H, J=8.8 Hz), 7.32-7.50 (m, 4H), 5.34 (s, 2H), 4.20 (dd, 1H, J=10.8 Hz, J=5.6 Hz), 4.08 (dd, 1H, J=10.8 Hz, J=5.6 Hz), 1.61 (s, 3H).

### (R)-2-amino-2-(S-(4-(biphenyl-3-ylmethoxy)-3-(trifluoromethyl)phenyl)-1H-imidazol-2-yl)propyl dihydrogen phosphate

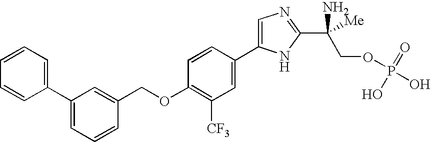

The final product was purified by reverse phase preparative HPLC, then lyophilized to dryness to obtain TFA salt of the product. The product was obtained as a white solid in 30% (33 mg) yield from the alcohol precursor. MS (ESI, M+H⁺)=548.7; ¹H NMR (400 MHz, DMSO-d₆) δ 8.80 (br s, 2H), 8.09 (d, 1H, J=2.0 Hz), 8.00 (dd, 1H, J=8.8 Hz, J=2.0 Hz), 7.66 (br s, 2H), 7.60-7.67 (m, 3H), 7.33-7.43 (m, 6H), 5.36 (s, 2H), 4.00-4.50 (m, 2H), 1.60 (s, 3H).

### (R)-2-amino-2-(5-(4-(biphenyl-4-ylmethoxy)-3-(trifluoromethyl)phenyl)-1H-imidazol-2-yl)ethyl dihydrogen phosphate

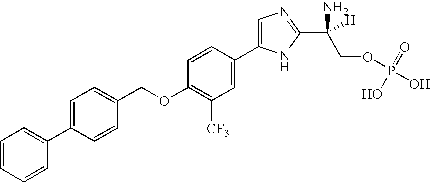

The final product was purified by reverse phase preparative HPLC, then lyophilized to dryness to obtain TFA salt of the product. The product was obtained as a white solid in 30% (33 mg) yield from the alcohol precursor. MS (ESI, M+H⁺)=534.1; ¹H NMR (400 MHz, DMSO-d₆) δ 8.07 (d, 1H, J=1.2 Hz), 8.00 (dd, 1H, J=8.6 Hz, J=1.6 Hz), 7.65-7.75 (m, 5H), 7.54 (d, 2H, J=8.6 Hz), 7.34-7.50 (m, 4H), 5.34 (s, 2H), 4.62 (d, 1H, J=5.2), 4.18-4.30 (m, 2H).

### (R)-2-Amino-2-(5-(4-(biphenyl-4-ylmethoxy)-3-(trifluoromethyl)phenyl)-4-methyl-1H-imidazol-2-yl)propyl dihydrogen phosphate

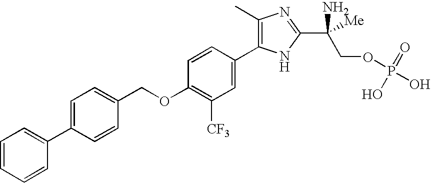

The final product was purified by reverse phase preparative HPLC, then lyophilized to dryness to obtain TFA salt of the product. MS (ESI, M+H⁺)=562.6

### (R)-2-Amino-2-(5-(4-(biphenyl-4-ylmethoxy)phenyl)-1H-imidazol-2-yl)propyl dihydrogen phosphate

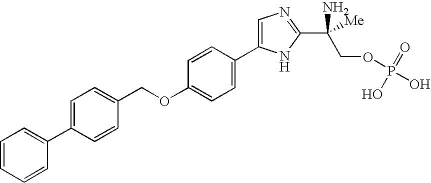

The final product was purified by reverse phase preparative HPLC, then lyophilized to dryness to obtain TFA salt of the product. MS (ESI, M+H⁺)=480.1

### (S)-2-Amino-2-(4-(4-(biphenyl-4-ylmethoxy)-3-(trifluoromethyl)phenyl)-5-methyloxazol-2-yl)propyl-dihydrogen phosphate

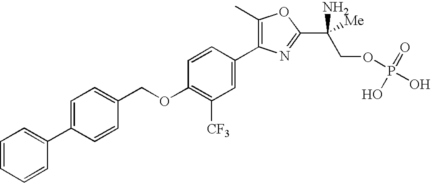

The final product was purified by reverse phase preparative HPLC, then lyophilized to dryness to obtain TFA salt of the product. MS (ESI, M+H⁺)=563.3

### (S)-2-amino-2-(4-(4-(biphenyl-4-ylmethoxy)-3-(trifluoromethyl)phenyl)thiazol-2-yl)propyl dihydrogen phosphate

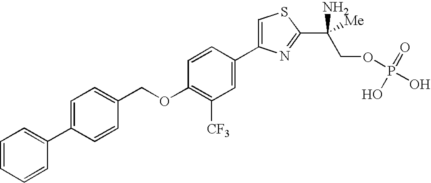

The final product was purified by reverse phase preparative HPLC, then lyophilized to dryness to obtain TFA salt of the product. The product was obtained as a white solid in 22% (20 mg) yield from the alcohol precursor. MS (ESI, M+H⁺)=565.6; ¹H NMR (400 MHz, DMSO-d₆) δ 8.24 (d, 1H, J=1.6 Hz), 8.22 (s, 1H), 8.18 (dd, 1H, J=8.8 Hz, J=2.0 Hz), 7.57-7.67 (m, 4H), 7.47 (d, 2H, J=8.4 Hz), 7.34-7.44 (m, 3H), 7.29 (t, 1H, J=7.6 Hz), 5.30 (s, 2H), 4.01-4.18 (m, 2H), 1.63 (s, 3H).

### (S)-2-Amino-2-(5-(4-(biphenyl-4-ylmethoxy)-3-(trifluoromethyl)phenyl)thiazol-2-yl)propyl-dihydrogen phosphate

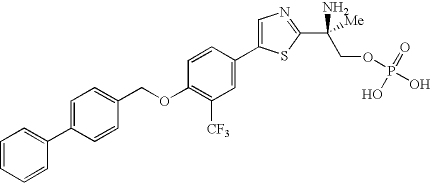

The final product was purified by reverse phase preparative HPLC, then lyophilized to dryness to obtain TFA salt of the product. MS (ESI, M+H⁺)=565.3

### Example 12

### Lymphopenia Assay

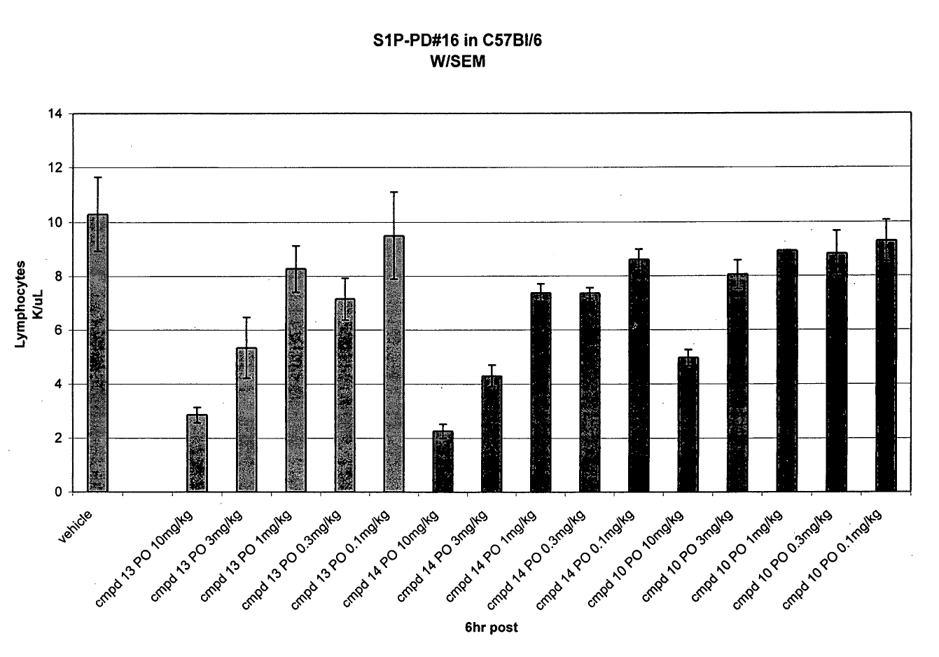

Several of the compounds described herein were evaluated for the ability to induce lymphopenia in mice. Male C57Bl/6 mice were divided into groups of three. A control group received the 3% BSA vehicle only. The other groups received a single dose of either a specified dose of test compound in vehicle administered orally (PO). After 6 hours, the mice were anesthesized with isoflurane and approximately 250 μL of blood was removed from the retroorbital sinus and collected in an EDTA microtainer, mixed with an anticoagulant and placed on a tilt table until complete blood count (CBC) analysis. FIG. 1 shows the results of the analysis for total lymphocyte count for different doses of compounds 10, 13 and 14. The results show that all three compounds, when dosed orally, are able to induce lymphopenia in mice relative to control.

### Example 13

### Binding to S1P1 or S1P3 Receptors The ability of several of the compounds described herein to bind to the S1P1 or S1P3 receptor was also tested as follows.

For the membrane preparation, plasmid DNA was transfected into HEK 293 T cells using the FuGENE 6 transfection protocol (publicly available by Roche). Briefly, subconfluent monolayers of HEK 293 T cells were transfected with the DNA mixture containing FuGENE 6 (using a 1:3 ratio). The dishes containing the cells were then placed in a tissue culture incubator (5% CO₂, 37° C.). The cells were harvested 48 hours after addition of the DNA by scraping in HME buffer (in mM: 20 HEPES, 5 MgC₁₂, 1 EDTA, pH 7.4, 1 mM PMSF) containing 10% sucrose on ice, and disrupted using a Dounce homogenizer. After centrifugation at 800×g, the supernatant was diluted with HME without sucrose and centrifuged at 17,000×g for 1 hour. This crude membrane pellet was resuspended in HME with sucrose, aliquoted, and snap-frozen by immersion in liquid nitrogen. The membranes were stored at −70 C. Protein concentration was determined spectroscopically by Bradford protein assay.

For the binding assay, [³³P]sphingosine 1-phosphate (obtained from American Radiolabeled Chemicals, Inc) was added to membranes in 200 μl in 96-well plates with assay concentrations of 2.5 pM [³³P]sphingosine 1-phosphate, 4 mg/ml BSA, 50 mM HEPES, pH 7.5, 100 mM NaCl, 5 mM MgCl2, and 5 μg of protein. Binding was performed for 60 minutes at room temperature with gentle mixing and terminated by collecting the membranes onto GF/B filter plates. After drying the filter plates for 10 minutes, 50 μl of Microscint 40 was added to each well, and filter-bound radionuclide was measured on a Packard Top Count. Nonspecific binding was defined as the amount of radioactivity remaining in the presence of excess of unlabeled SIP. The results for the foregoing binding assays are presented in Table 1 provided below.

TABLE 1
IC50 Values for Binding to S1P1 or S1P3 Receptors

| COMPOUND NO. | S1P1 IC₅₀ (nM) | S1P3 IC₅₀ (nM) |
|---|---|---|
| 203 | ** | * |
| 204 | ** | * |
| 76 | *** | * |
| 86 | **** | *** |
| 208 | * | * |
| 209 | ** | * |
| 184 | **** | *** |
| 244 | **** | ** |
| 210 | ** | * |
| 110 | **** | **** |
| 171 | ** | * |
| 88 | *** | ** |
| 91 | *** | ** |
| 87 | **** | *** |
| 86 | **** | *** |
| 216 | **** | ** |
| 92 | **** | ** |
| 45 | **** | *** |
| 192 | * | * |
| 173 | **** | ** |
| 184 | **** | *** |
| 185 | *** | ** |
| 188 | ** | ** |
| 189 | ** | * |
| 190 | **** | * |
| 191 | **** | *** |
| 193 | *** | * |
| 194 | *** | *** |
| 195 | **** | ** |
| 196 | **** | **** |
| 186 | **** | *** |
| 187 | **** | **** |
| 199 | **** | *** |
| 200 | ** | * |
| 201 | **** | ** |
| 202 | **** | *** |
| 213 | *** | *** |
| 205 | **** | ** |
| 207 | **** | ** |
| 214 | **** | * |
| 215 | * | * |
| 220 | *** | ** |
| 217 | **** | *** |
| 218 | * | * |
| 219 | **** | *** |
| 221 | *** | ** |
| 222 | **** | ** |

Key
IC₅₀
* limited receptor interaction (IC₅₀ > 10,000 nM)
** some receptor interaction (10,000 nM ≧ IC₅₀ > 1,000 nM)
*** good receptor interaction (1,000 nM ≧ IC₅₀ > 100 nM)
**** very good receptor interaction (100 nM ≧ IC₅₀ ≧ 0.001 nM)

Equivalents

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including reaction times, reaction size/volume, and experimental reagents, such as solvents, catalysts, pressures, atmospheric conditions, e.g., nitrogen atmosphere, and reducing/oxidizing agents, etc., with art-recognized alternatives and using no more than routine experimentation, are within the scope of the present application.

It is to be understood that wherever values and ranges are provided herein, e.g., in ages of subject populations, dosages, and blood levels, all values and ranges encompassed by these values and ranges, are meant to be encompassed within the scope of the present invention. Moreover, all values that fall within these ranges, as well as the upper or lower limits of a range of values, are also contemplated by the present application.

We claim:

1. A compound of Formula XII: 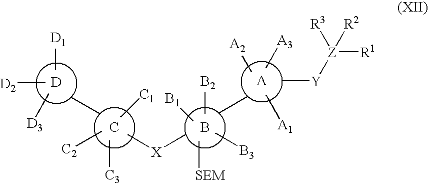
wherein:
SEM represents a selectivity enhancing moiety;
rings A, B, C, D are independently selected from the group consisting of substituted or unsubstituted carbocyclic rings and substituted or unsubstituted heterocyclic rings, which may contain one or more heteroatoms and may be saturated or unsaturated;
A₁, A₂, A₃, B₁, B₂, B₃, C₁, C₂, C₃, D₁, D₂, and D₃ are each independently selected from the group consisting of hydrogen, halogen, cyano, straight chain or branched aliphatic, straight chain or branched alkoxy, straight chain or branched halo-alkyl, straight chain or branched halo-alkoxy, alkoxy-alkyl, hydroxyl-alkyl, carboxy-alkyl, alkyl-SO₂ alkylcarbonyl, thioether, alkylsulfonyl, alkylcarbonylamino, alkylaminocarbonyl, alkyloxycarbonyl, alkylcarbonyloxy, substituted or unsubstituted aryl, substituted or unsubstituted heteroaryl, —OH, —C(O)-alkyl, —C(O)-halo-alkyl, —C(O)O-alkyl, —C(O)O-halo-alkyl, —CONH₂, —CONH-alkyl, —CON-dialkyl, —CONH—halo-alkyl, —CON-halo-dialkyl, -alkyl-CONH-alkyl, -alkyl-CON-dialkyl, halo-alkyl-CONH-alkyl, halo-alkyl-CONH- halo-alkyl, alkyl-CONH- halo-dialkyl, -alkyl-hydroxyl, -alkyl-hydroxyl-alkyl, -halo-alkyl-hydroxyl-alkyl, -alkyl-hydroxyl-halo-alkyl, -halo-alkyl-hydroxyl-halo-alkyl, substituted or unsubstituted alkyl-OR¹⁴, substituted or unsubstituted haloalkyl-OR¹⁴, —OR¹⁴, and N(R)R′; or taken together A₃ and B₃ may form a substituted or unsubstituted carbocyclic ring or substituted or unsubstituted heterocyclic ring, which may contain one or more heteroatoms and may be saturated or unsaturated; or taken together C₂ and D₂ may form a substituted or unsubstituted carbocyclic ring or substituted or unsubstituted heterocyclic ring, which may contain one or more heteroatoms and may be saturated or unsaturated;
R and R′ are each independently selected from the group consisting of hydrogen, cyano, straight chain or branched alkyl, straight chain or branched alkoxy, straight chain or branched halo-alkyl, straight chain or branched halo-alkoxy, alkoxy-alkyl, hydroxyl-alkyl, and carboxy-alkyl; or taken together R and R′ may form a substituted or unsubstituted carbocyclic ring or substituted or unsubstituted or heterocyclic ring, which may contain one or more heteroatoms and may be saturated or unsaturated; or taken together with the N to which they are attached R and R′ may form a moiety selected from the group consisting of substituted straight chain or cyclic guanyl, straight chain or cyclic guanidine, straight chain or cyclic urea, straight chain or cyclic thiourea, straight chain or cyclic carbamate, and straight chain or cyclic thiocarbamate;
Z is independently selected from the group consisting of C or N;
R′ is a phosphate derivative, a phosphate mimic or a phosphate precursor;
R² and R³ are each independently selected from the group consisting of hydrogen, hydroxyl, halogen, cyano, straight chain or branched alkyl, alkyl-OR⁹, halo-alkyl-OR⁹, alkoxy-OR⁹, alkyl-OC(O)R⁹, halo-alkyl-OC(O)R⁹, alkoxy-OC(O)R⁹, carbocyclic rings, heterocyclic rings which may contain one or more heteroatoms, alkyl-NR⁹R¹⁰, halo-alkyl-NR⁹R¹⁰, and alkoxy-NR⁹R¹⁰, all of which may be optionally substituted with OH, halogen, NHR⁹, NR⁹R¹⁰, straight chain or branched alkoxy, straight chain or branched halo-alkyl, straight chain or branched halo-alkoxy, alkoxy-alkyl, hydroxyl-alkyl, or carboxy-alkyl; or taken together R² and R³ may form a substituted or unsubstituted carbocyclic ring or a substituted or unsubstituted heterocyclic ring, which may contain one or more heteroatoms and may be saturated or unsaturated; or taken together R² and A₁ may form a substituted or unsubstituted C₄-C₁₀ fused carbocyclic ring or substituted or unsubstituted C₄-C₁₀ fused heterocyclic rings, which may contain one or more heteroatoms and may be saturated or unsaturated;
R⁹ and R¹⁰ are independently selected from the group consisting of hydrogen, halogen, cyano, straight chain or branched alkyl, straight chain or branched alkoxy, straight chain or branched halo-alkyl, straight chain and branched halo-alkoxy, —C(O)alkyl, —C(O)NH-alkyl, —C(O)N-dialkyl, —C(O)aryl, —C(O)NH-aryl, —C(O)N-alkyl-aryl, —C(O)N-diaryl, —C(O)heteroaryl, —C(O)NH-heteroaryl, —C(O)N-carbocycle, substituted or unsubstituted carbocyclic rings, and substituted or unsubstituted heterocyclic rings, which may contain one or more heteroatoms and may be saturated or unsaturated; or taken together R⁹ and R¹⁰ may form a substituted or unsubstituted carbocyclic ring or a substituted or unsubstituted heterocyclic ring, which may contain one or more heteroatoms and may be saturated or unsaturated; or R⁹ or R¹⁰ together with A₁ may form a substituted or unsubstituted C₄-C₁₀ fused carbocyclic ring or substituted or unsubstituted C₄-C₁₀ fused heterocyclic rings, which may contain one or more heteroatoms and may be saturated or unsaturated;
Y is independently selected from the group consisting of (CR¹¹R 12)ₙ and (CR¹¹R¹²)ₙNR¹³;
R¹¹, R¹², and R¹³ are independently selected from the group consisting of hydrogen, halogen, cyano, and straight chain or branched alkyl, all of which may be optionally substituted with OH, halogen, straight chain or branched alkoxy, straight chain or branched halo-alkyl, and straight chain and branched halo-alkoxy; or R¹³ may form a 3-8-membered ring together with either R¹¹ or R¹² and the atom to which they are attached;
n is an integer from 0 to 3;
X is selected from the group consisting of 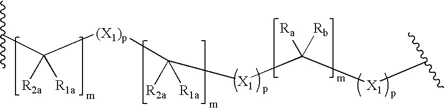
wherein each m is independently selected from an integer between 0 and 6; each p is independently selected from 0 or 1; each X₁ is independently selected from the group consisting of CR¹⁴R¹⁵, NR¹⁴, S, and O, —S(O), —S(O)₂, —OS(O)₂, —OS(O)₂O—, —C(O), C(OH), —C(O)O—, a substituted or unsubstituted aromatic, a substituted or unsubstituted heteroaromatic, and any combination thereof, in any orientation; each Rₐ and R_{b} are independently selected from the group consisting of hydrogen, cyano, halogen, alkyl, halo-alkyl, —OH, —CO—, straight chain or branched alkoxy, straight chain or branched halo-alkyl, straight chain or branched halo-alkoxy, alkoxy-alkyl, hydroxyl-alkyl, -alkyl-hydroxyl, -alkyl-hydroxyl-alkyl, -halo-alkyl-hydroxyl-alkyl, -alkyl-hydroxyl-halo-alkyl, -halo-alkyl-hydroxyl-halo-alkyl, carboxy-alkyl, alkyl-SO₂, alkylcarbonyl, thioether, alkylsulfonyl, alkylcarbonylamino, alkylaminocarbonyl, alkyloxycarbonyl, alkylcarbonyloxy, substituted or unsubstituted aryl, and substituted or unsubstituted heteroaryl, all of which may be optionally substituted with OH, halogen, straight chain or branched alkoxy, straight chain or branched halo-alkyl, straight chain and branched halo-alkoxy, alkoxy-alkyl, hydroxyl-alkyl, carboxy-alkyl, substituted or unsubstituted carbocyclic rings, and substituted or unsubstituted heterocyclic rings, which may contain one or more heteroatoms and may be saturated or unsaturated; or each Rₐ and R_{b} may form a 3-10-membered ring together with the carbon to which they are both attached;
each R₁ₐ and R₂ₐ are independently selected from the group consisting of hydrogen, cyano, halogen, alkyl, halo-alkyl, —OH, —CO—, straight chain or branched alkoxy, straight chain or branched halo-alkyl, straight chain or branched halo-alkoxy, alkoxy-alkyl, hydroxyl-alkyl, -alkyl-hydroxyl, -alkyl-hydroxyl-alkyl, -halo-alkyl-hydroxyl-alkyl, -alkyl-hydroxyl-halo-alkyl, -halo-alkyl-hydroxyl-halo-alkyl, carboxy-alkyl, alkyl-SO₂, alkylcarbonyl, thioether, alkylsulfonyl, alkylcarbonylamino, alkylaminocarbonyl, alkyloxycarbonyl, alkylcarbonyloxy, substituted or unsubstituted aryl, and substituted or unsubstituted heteroaryl, all of which may be optionally substituted with OH, halogen, straight chain or branched alkoxy, straight chain or branched halo-alkyl, straight chain and branched halo-alkoxy, alkoxy-alkyl, hydroxyl-alkyl, carboxy-alkyl, substituted or unsubstituted carbocyclic rings, and substituted or unsubstituted heterocyclic rings, which may contain one or more heteroatoms and may be saturated or unsaturated; or each R₁ₐ and R₂ₐ may form a 3-10-membered ring together with the carbon to which they are both attached; and
each R¹⁴ and R¹⁵ is independently selected from the group consisting of hydrogen, halogen, cyano, straight chain or branched alkyl, straight chain or branched alkoxy, straight chain or branched halo-alkyl, straight chain or branched halo-alkoxy, alkoxy-alkyl, hydroxyl-alkyl, alkyl-SO₂, and carboxy-alkyl; or each R¹⁴ or R¹⁵ may form a 3-8-membered ring together with B₁, Rₐ, R_{b}, R₁ₐ, or R₂ₐ and the atoms to which they are attached.

2. The compound of claim 1, wherein R¹ is L₁-O—H or L₁-O-L₂, wherein L₁ is a linking moiety and L₂ is a labile moiety.

3. The compound of claim 2, wherein R¹ is selected from the group consisting of -alkyl-OH, -halo-alkyl-OH, alkoxy-OH, -alkyl-OCOR⁴, -halo-alkyl-OCOR⁴, -alkoxy-OCOR⁴, -alkyl-OC(O)NR⁴R⁵, -halo-alkyl-OC(O)NHR⁴R⁵, -alkoxy-OC(O)NR⁴R⁵, —(CH₂)_{q}CO₂R⁶, and —(CH₂)ₙCH₂=CHC(O)OR⁶, wherein
q is an integer between 0 and 4;
R⁴and R⁵ are independently selected from the group consisting of hydrogen, straight chain or branched C₁-C₆-alkyl, all of which may be optionally substituted with OH, halogen, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl, substituted or unsubstituted C₃-C₁₀ carbocyclic rings, and substituted or unsubstituted C₃-C₁₀ heterocyclic rings, which may contain one or more heteroatoms and may be saturated or unsaturated; and
R⁶ is selected from the group consisting of hydrogen, straight chain or branched C₁-C₆-alkyl, straight chain or branched halo-C₁-C₆-alkyl, substituted or unsubstituted aryl group, and a prodrug derivatizing moiety (PDM).

4. The compound of claim 1, wherein R¹ is selected from the group consisting of —(CH₂)_{q}OPO₂R⁷R⁸, —(CH₂)_{q}OPO₃R⁷R⁸, and —(CH₂)_{q}OPO₂(S)R⁷R⁸, wherein
q is an integer between 0 and 4; and
R⁷ and R⁸ are each independently selected from the group consisting of hydrogen, straight chain or branched C₁-C₆-alkyl, straight chain or branched halo-C₁-C₆-alkyl, substituted or unsubstituted aryl group, and a prodrug derivatizing moiety (PDM).

5. The compound of claim 1, wherein R¹ is -L₁-Z₂, wherein L₁ is a linking moiety and Z₂ is a non-hydrolyzable moiety covalently bonded to L₁.

6. The compound of claim 5, wherein R¹ is selected from the group consisting of —(CH₂)_{q}CH₂PO₃R⁷R⁸, and —(CH₂)_{q}C(Y₁)(Y₂)PO₃R⁷R⁸, wherein
q is an integer between 0 and 4;
Y₁ and Y₂ are independently selected from the group consisting of hydrogen, straight chain or branched C₁-C₆-alkyl, all of which may be optionally substituted with OH, halogen, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl, substituted or unsubstituted C₃-C₁₀ carbocyclic rings, and substituted or unsubstituted C₃-C₁₀ heterocyclic rings, which may contain one or more heteroatoms and may be saturated or unsaturated; and
R⁷ and R⁸ are each independently selected from the group consisting of hydrogen, straight chain or branched C₁-C₆-alkyl, straight chain or branched halo-C₁-C₆-alkyl, substituted or unsubstituted aryl group, and a prodrug derivatizing moiety (PDM).

7. The compound of claim 6, wherein the PDM is selected from the group consisting of:

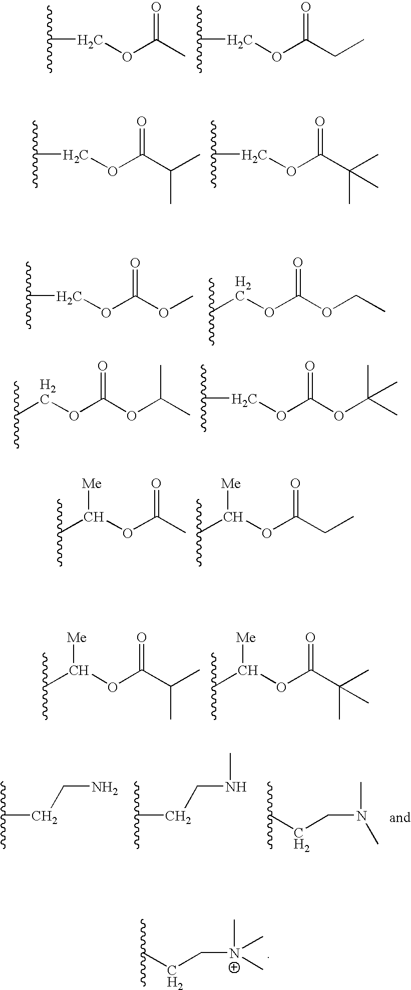

8. The compound of claim 1, wherein taken together R² and R³ form a substituted or unsubstituted C₃-C₁₀ carbocyclic ring or a substituted or unsubstituted C₁-C₁₀ heterocyclic ring, which may contain one or more heteroatoms and may be saturated or unsaturated, said ring contains at least one halogen.

9. The compound of claim 1, wherein each of A, B, C, D is independently selected from the group consisting of an aromatic ring and a heteroaromatic ring.

10. The compound of claim 1, wherein X is independently selected from the group consisting of straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl, C₁-C₆-alkyl-SO₂ alkylcarbonyl, thioether, alkylsulfonyl, alkylcarbonylamino, alkylaminocarbonyl, alkyloxycarbonyl, alkylcarbonyloxy, substituted or unsubstituted aromatic, and substituted or unsubstituted heteroaromatic; or taken together with either B₂ or C₁, R¹⁵ may form a substituted or unsubstituted C₃-C₁₀ carbocyclic ring or substituted or unsubstituted C₃-C₁₀ heterocyclic ring, which may contain one or more heteroatoms and may be saturated or unsaturated

11. The compound of claim 10, wherein X is selected from the group consisting of —CH₂NR¹⁴—, —CH₂NR¹⁴(CO)—, —CHFNR¹⁴—, —CHFNR¹⁴(CO)—, —CF₂NR¹⁴—, —CF₂NR¹⁴(CO)—, —CH₂(CO)—, —CHF(CO)—, —CF₂(CO)—, —(CO)CH₂—, —(CO)CHF—, —(CO)CF₂—, —CH₂(CHOH)—, —CHF(CHOH)—, —CF₂(CHOH)—, —(CHOH)CH₂—, —(CHOH) CHF—, —(CHOH)CF₂—, —NH(CO)—, —(CO)NH—, —(CO)—, —(CO)₂—, —O—, —S—, —SO—, —SO₂—, —CH₂O—, —CH₂CH₂O—, —CH₂OCH₂—, —OCH₂CH₂—, —OCH₂O—, —CH₂S—, —CH₂SO—, —CH₂SO₂—, —OCH₂—, —SCH₂—, —SOCH₂—, —SO₂CH₂—, —CHFO—, —CHFS—, —CHFSO—, —CHFSO₂—, —OCHF—, —SCHF—, —SOCHF—, —SO₂CHF—, —CF₂O—, —CF₂S—, —CF₂SO—, —CF₂SO₂—, —OCF₂—, —SCF₂—, —SOCF₂—, —SO₂CF₂—, —SO₂CF₂—, —NR¹⁴SO₂—, —SO₂NR¹⁴—, —CF₂—, —CF₂CF₂—, a aromatic group, and a heteroaromatic group.

12. The compound of claim 1 having the following formula: 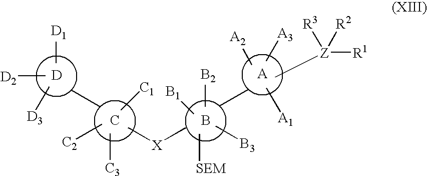
wherein:
SEM represents a selectivity enhancing moiety;
rings A, B, C, D are independently selected from the group consisting of any five- or six-membered aromatic or heteroaromatic, and isomers and tautomers thereof;
A₁, A₂, A₃, B₁, B₂, B₃, C₁, C₂, C₃, D₁, D₂, and D₃ are each independently selected from the group consisting of hydrogen, halogen, cyano, straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl, C₁-C₆-alkyl-SO₂ alkylcarbonyl, thioether, alkylsulfonyl, alkylcarbonylamino, alkylaminocarbonyl, alkyloxycarbonyl, alkylcarbonyloxy, substituted or unsubstituted aryl, substituted or unsubstituted heteroaryl, —OH, —CO₂-alkyl, —CO₂-halo-alkyl, —CONH₂, —CONH-alkyl, —CON-dialkyl, —CONH-halo-alkyl, —CON-halo-dialkyl, -alkyl-CONH-alkyl, -alkyl-CON-dialkyl, halo-alkyl-CONH- alkyl, halo-alkyl-CONH- halo-alkyl, alkyl-CONH-halo-dialkyl, —C₁-C₆-alkyl-hydroxyl, —C₁-C₆-alkyl-hydroxyl-alkyl, —C₁-C₆-halo-alkyl-hydroxyl-alkyl, —C₁-C₆-alkyl-hydroxyl-halo-alkyl, —C₁-C₆-halo-alkyl-hydroxyl-halo-alkyl, and N(R)R′; or taken together A₃ and B₃ may form a substituted or unsubstituted C₃-C₁₀ carbocyclic ring or substituted or unsubstituted C₃-C₁₀ heterocyclic ring, which may contain one or more heteroatoms and may be saturated or unsaturated; or taken together C₂ and D₂ may form a substituted or unsubstituted C₃-C₁₀ carbocyclic ring or substituted or unsubstituted C₃-C₁₀ heterocyclic ring, which may contain one or more heteroatoms and may be saturated or unsaturated;
R and R′ are each independently selected from the group consisting of hydrogen, halogen, cyano, straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, and carboxy-C₁-C₆-alkyl; or taken together R and R′ may form a substituted or unsubstituted C₃-C₁₀ carbocyclic ring or substituted or unsubstituted C₃-C₁₀ or heterocyclic ring, which may contain one or more heteroatoms and may be saturated or unsaturated; or taken together with the N to which they are attached R and R′ may form a moiety selected from the group consisting of substituted straight chain or cyclic guanyl, straight chain or cyclic guanidine, straight chain or cyclic urea, straight chain or cyclic thiourea, straight chain or cyclic carbamate, and straight chain or cyclic thiocarbamate;
Z is independently selected from the group consisting of C or N;
R¹ is a phospahate, a phosphate mimic or a phosphate precursor;
R² and R³ are each independently selected from the group consisting of hydrogen, halogen, cyano, straight chain or branched C₁-C₆-alkyl, alkyl-OR⁹, halo-alkyl-OR⁹, alkoxy-OR⁹, alkyl-OC(O)R⁹, halo-alkyl-OC(O)R⁹, alkoxy-OC(O)R⁹, alkyl-NR⁹R¹⁰, halo-alkyl-NR⁹R¹⁰, and alkoxy-NR⁹R¹⁰, all of which may be optionally substituted with OH, halogen, NHR⁹, NR⁹R¹⁰, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, or carboxy-C₁-C₆-alkyl; or taken together R² and R³ may form a substituted or unsubstituted C₃-C₁₀ carbocyclic ring or a substituted or unsubstituted C₃-C₁₀ heterocyclic ring, which may contain one or more heteroatoms and may be saturated or unsaturated; or taken together R² and A₁ may form a substituted or unsubstituted C₄-C₁₀ fused carbocyclic ring or substituted or unsubstituted C₄-C₁₀ fused heterocyclic rings, which may contain one or more heteroatoms and may be saturated or unsaturated;
R⁹ and R¹⁰ are independently selected from the group consisting of hydrogen, halogen, cyano, straight chain or branched C₁-C₆-alkyl, straight chain or branched C₁-C₆-alkoxy, straight chain or branched halo-C₁-C₆-alkyl, straight chain and branched halo-C₁-C₆-alkoxy, substituted or unsubstituted C₃-C₁₀ carbocyclic rings, and substituted or unsubstituted C₃-C₁₀ heterocyclic rings, which may contain one or more heteroatoms and may be saturated or unsaturated; or taken together R⁹ and R¹⁰ may form a substituted or unsubstituted C₃-C₁₀ carbocyclic ring or a substituted or unsubstituted C₃-C₁₀ heterocyclic ring, which may contain one or more heteroatoms and may be saturated or unsaturated; or R9 or R together with A₁ may form a substituted or unsubstituted C₄-C₁₀ fused carbocyclic ring or substituted or unsubstituted C₄-C₁₀ fused heterocyclic rings, which may contain one or more heteroatoms and may be saturated or unsaturated;
X is selected from the group consisting of 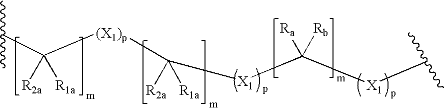
wherein each m is independently selected from an integer between 0 and 6; each p is independently selected from 0 or 1; each X₁ is independently selected from the group consisting of CR¹⁴R¹⁵, NR¹⁴, S, and O, —S(O), —S(O)₂, —OS(O)₂, —OS(O)₂O—, —C(O), C(OH), —C(O)O—, a substituted or unsubstituted aromatic, a substituted or unsubstituted heteroaromatic, and any combination thereof, in any orientation; each Rₐ and R_{b} are independently selected from the group consisting of hydrogen, cyano, and straight chain or branched C₁-C₆-alkyl, all of which may be optionally substituted with OH, halogen, straight chain or branched C₁-C₆-alkoxy, straight chain or branched halo-C₁-C₆-alkyl, straight chain and branched halo-C₁-C₆-alkoxy, C₁-C₆-alkoxy-C₁-C₆-alkyl, hydroxyl-C₁-C₆-alkyl, carboxy-C₁-C₆-alkyl, substituted or unsubstituted C₃-C₁₀ carbocyclic rings, and substituted or unsubstituted C₃-C₁₀ heterocyclic rings, which may contain one or more heteroatoms and may be saturated or unsaturated; or each Rₐ and R_{b} may form a 3-8-membered ring together with the carbon to which they are both attached;
each R₁ₐ and R₂ₐ are independently selected from the group consisting of hydrogen, halogen, cyano, and straight chain or branched C₁-C₆-alkyl, all of which may be optionally substituted with OH, halogen, straight chain or branched C₁-C₆-alkoxy, straight chain or branched halo-C₁-C₆-alkyl, straight chain and branched halo-C₁-C₆-alkoxy, C₁-C₆-alkoxy-C₁-C₆-alkyl, hydroxyl-C₁-C₆-alkyl, carboxy-C₁-C₆-alkyl, substituted or unsubstituted C₃-C₁₀ carbocyclic rings, and substituted or unsubstituted C₃-C₁₀ heterocyclic rings, which may contain one or more heteroatoms and may be saturated or unsaturated; or each R₁ₐ and R₂ₐ may form a 3-8-membered ring together with the carbon to which they are both attached; and
each R¹⁴ and R¹⁵ is independently selected from the group consisting of hydrogen, halogen, cyano, straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, and carboxy-C₁-C₆-alkyl; or each R¹⁴ or R¹⁵ may form a 3-8-membered ring together with B₁, Rₐ, R_{b}, R₁ₐ, or R₂ₐ and the atoms to which they are attached.

13. The compound of claim 12, wherein R¹ is L₁-O—H or L₁-O-L₂, wherein L₁ is a linking moiety and L₂ is a labile moiety.

14. The compound of claim 13, wherein R¹ is selected from the group consisting of -alkyl-OH, -halo-alkyl-OH, alkoxy-OH, -alkyl-OCOR⁴, -halo-alkyl-OCOR⁴, -alkoxy-OCOR⁴, -alkyl-OC(O)NR⁴R⁵, -halo-alkyl-OC(O)NHR⁴R⁵, -alkoxy-OC(O)NR⁴R⁵, —(CH₂)_{q}CO₂R⁶, and —(CH₂),CH₂═CHC(O)OR⁶, wherein
q is an integer between 0 and 4;
R⁴and R⁵ are independently selected from the group consisting of hydrogen, straight chain or branched C₁-C₆-alkyl, all of which may be optionally substituted with OH, halogen, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl, substituted or unsubstituted C₃-CIO carbocyclic rings, and substituted or unsubstituted C₃-C₁₀ heterocyclic rings, which may contain one or more heteroatoms and may be saturated or unsaturated; and
R⁶ is selected from the group consisting of hydrogen, straight chain or branched C₁-C₆-alkyl, straight chain or branched halo-C₁-C₆-alkyl, substituted or unsubstituted aryl group, and a prodrug derivatizing moiety (PDM).

15. The compound of claim 12, wherein R¹ is selected from the group consisting of —(CH₂)_{q}OPO₂R⁷R⁸, —(CH₂)_{q}OPO₃R⁷R⁸, and —(CH₂)_{q}OPO₂(S)R⁷R⁸, wherein
q is an integer between 0 and 4; and
R⁷ and R⁸ are each independently selected from the group consisting of hydrogen, straight chain or branched C₁-C₆-alkyl, straight chain or branched halo-C₁-C₆-alkyl, substituted or unsubstituted aryl group, and a prodrug derivatizing moiety (PDM).

16. The compound of claim 12, wherein R¹ is -L₁-Z₂, wherein L₁ is a linking moiety and Z₂ is a non-hydrolyzable moiety covalently bonded to L₁.

17. The compound of claim 16, wherein R¹ is selected from the group consisting of —(CH₂)_{q}CH₂PO₃R⁷R⁸, and —(CH₂)_{q}C(Y₁)(Y₂)PO₃R⁷R⁸, wherein
q is an integer between 0 and 4;
Y₁ and Y₂ are independently selected from the group consisting of hydrogen, straight chain or branched C₁-C₆-alkyl, all of which may be optionally substituted with OH, halogen, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl, substituted or unsubstituted C₃-C₁₀ carbocyclic rings, and substituted or unsubstituted C₃-C₁₀ heterocyclic rings, which may contain one or more heteroatoms and may be saturated or unsaturated; and
R⁷ and R⁸ are each independently selected from the group consisting of hydrogen, straight chain or branched C₁-C₆-alkyl, straight chain or branched halo-C₁-C₆-alkyl, substituted or unsubstituted aryl group, and a prodrug derivatizing moiety (PDM).

18. The compound of claim 17, wherein the PDM is selected from the group consisting of:

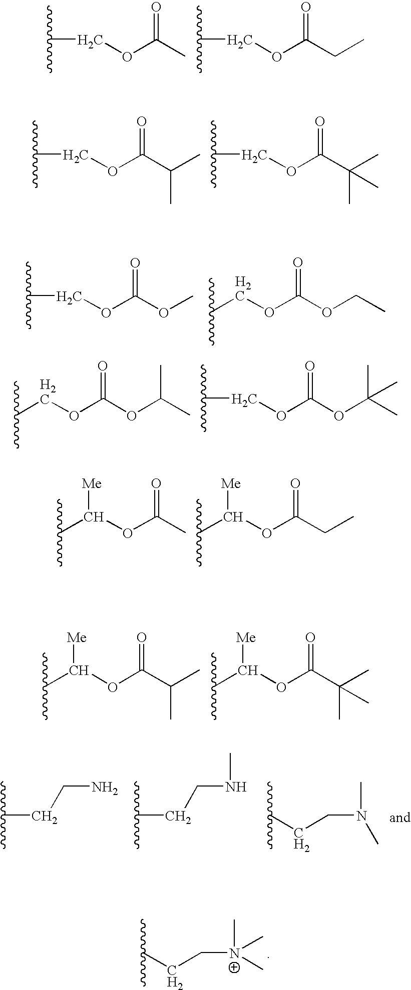

19. The compound of claim 12, wherein taken together R² and R³ form a substituted or unsubstituted C₃-C₁₀ carbocyclic ring or a substituted or unsubstituted C₁-C₁₀ heterocyclic ring, which may contain one or more heteroatoms and may be saturated or unsaturated, said ring contains at least one halogen.

20. The compound of claim 12, wherein each of A, B, C, D is independently selected from the group consisting of an aromatic ring and a heteroaromatic ring.

21. The compound of claim 12, wherein X is independently selected from the group consisting of straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl, C₁-C₆-alkyl-SO₂ alkylcarbonyl, thioether, alkylsulfonyl, alkylcarbonylamino, alkylaminocarbonyl, alkyloxycarbonyl, alkylcarbonyloxy, substituted or unsubstituted aromatic, and substituted or unsubstituted heteroaromatic; or taken together with, either B₂ or C₁, R¹⁵ may form a substituted or unsubstituted C₃-C₁₀ carbocyclic ring or substituted or unsubstituted C₃-C₁₀ heterocyclic ring, which may contain one or more heteroatoms and may be saturated or unsaturated.

22. The compound of claim 21, wherein X is selected from the group consisting of —CH₂NR¹⁴—, —CH₂NR¹⁴(CO)—, —CHFNR¹⁴—, —CHFNR¹⁴(CO)—, —CF₂NR¹⁴—, —CF₂NR¹⁴(CO)—, —CH₂(CO)—, —CHF(CO)—, —CF₂(CO)—, —(CO)CH₂—, —(CO)CHF—, —(CO)CF₂—, —CH₂(CHOH)—, —CHF(CHOH)—, —CF₂(CHOH)—, —(CHOH)CH₂—, —(CHOH) CHF—, —(CHOH)CF₂—, —NH(CO)—, —(CO)NH—, —(CO)—, —(CO)₂—, —O—, —S—, —SO—, —SO₂—, —CH₂O—, —CH₂CH₂O—, —CH₂OCH₂—, —OCH₂CH₂—, —OCH₂O—, —CH₂S—, —CH₂SO—, —CH₂SO₂—, —OCH₂—, —SCH₂—, —SOCH₂—, —SO₂CH₂—, —CHFO—, —CHFS—, —CHFSO—, —CHFSO₂—, —OCHF—, —SCHF—, —SOCHF—, —SO₂CHF—, —CF₂O—, —CF₂S—, —CF₂SO—, —CF₂SO₂—, —OCF₂—, —SCF₂—, —SOCF₂—, —SO₂CF₂—, —SO₂CF₂—, —NR¹⁴SO₂—, —SO₂NR¹⁴—, —CF₂—, —CF₂CF₂—, a aromatic group, and a heteroaromatic group.

23. The compound of claim 1 having the following formula: 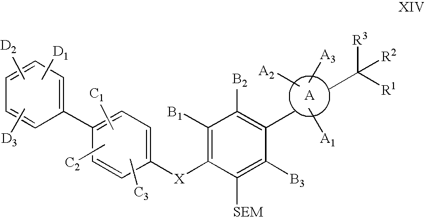
wherein:
SEM represents a selectivity enhancing moiety;
ring A is selected from the group consisting of any five- or six-membered aromatic or heteroaromatic, and isomers and tautomers thereof;
A₁, A₂, A₃, B₁, B₂, B₃, C₁, C₂, C₃, D₁, D₂, and D₃ are each independently selected from the group consisting of hydrogen, halogen, cyano, straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl, C₁-C₆-alkyl-SO₂ alkylcarbonyl, thioether, alkylsulfonyl, alkylcarbonylamino, alkylaminocarbonyl, alkyloxycarbonyl, alkylcarbonyloxy, substituted or unsubstituted aryl, substituted or unsubstituted heteroaryl, —OH, —CO₂-alkyl, —CO₂-halo-alkyl, —CONH₂, —CONH-alkyl, —CON-dialkyl, —CONH-halo-alkyl, —CON-halo-dialkyl, -alkyl-CONH-alkyl, -alkyl-CON-dialkyl, halo-alkyl-CONH-alkyl, halo-alkyl-CONH- halo-alkyl, alkyl-CONH-halo-dialkyl, —C₁-C₆-alkyl-hydroxyl, —C₁-C₆-alkyl-hydroxyl-alkyl, —C₁-C₆-halo-alkyl-hydroxyl-alkyl, —C₁-C₆-alkyl-hydroxyl-halo-alkyl, —C₁-C₆-halo-alkyl-hydroxyl-halo-alkyl, and N(R)R′; or taken together A₃ and B₃ may form a substituted or unsubstituted C₃-C₁₀ carbocyclic ring or substituted or unsubstituted C₃-C₁₀ heterocyclic ring, which may contain one or more heteroatoms and may be saturated or unsaturated; or taken together C₂ and D₂ may form a substituted or unsubstituted C₃-C₁₀ carbocyclic ring or substituted or unsubstituted C₃-C₁₀ heterocyclic ring, which may contain one or more heteroatoms and may be saturated or unsaturated;
R and R′ are each independently selected from the group consisting of hydrogen, halogen, cyano, straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, and carboxy-C₁-C₆-alkyl; or taken together R and R′ may form a substituted or unsubstituted C₃-C₁₀ carbocyclic ring or substituted or unsubstituted C₃-C₁₀ or heterocyclic ring, which may contain one or more heteroatoms and may be saturated or unsaturated; or taken together with the N to which they are attached R and R′ may form a moiety selected from the group consisting of substituted straight chain or cyclic guanyl, straight chain or cyclic guanidine, straight chain or cyclic urea, straight chain or cyclic thiourea, straight chain or cyclic carbamate, and straight chain or cyclic thiocarbamate;
R′ is a phosphate, a phosphate mimic or a phosphate precursor;
R² and R³ are each independently selected from the group consisting of hydrogen, halogen, cyano, straight chain or branched C₁-C₆-alkyl, alkyl-OR⁹, halo-alkyl-OR⁹, alkoxy-OR⁹, alkyl-OC(O)R⁹, halo-alkyl-OC(O)R⁹, alkoxy-OC(O)R⁹, alkyl-NR⁹R¹⁰, halo-alkyl-NR⁹R¹⁰, and alkoxy-NR⁹R¹⁰, all of which may be optionally substituted with OH, halogen, NHR⁹, NR⁹R¹⁰, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, or carboxy-C₁-C₆-alkyl; or taken together R² and R³ may form a substituted or unsubstituted C₃-C₁₀ carbocyclic ring or a substituted or unsubstituted C₃-C₁₀ heterocyclic ring, which may contain one or more heteroatoms and may be saturated or unsaturated; or taken together R² and A₁ may form a substituted or unsubstituted C₄-C₁₀ fused carbocyclic ring or substituted or unsubstituted C₄-C₁₀ fused heterocyclic rings, which may contain one or more heteroatoms and may be saturated or unsaturated;
R⁹ and R¹⁰ are independently selected from the group consisting of hydrogen, halogen, cyano, straight chain or branched C₁-C₆-alkyl, straight chain or branched C₁-C₆-alkoxy, straight chain or branched halo-C₁-C₆-alkyl, straight chain and branched halo-C₁-C₆-alkoxy, substituted or unsubstituted C₃-C₁₀ carbocyclic rings, and substituted or unsubstituted C₃-C₁₀ heterocyclic rings, which may contain one or more heteroatoms and may be saturated or unsaturated; or taken together R⁹ and R¹⁰ may form a substituted or unsubstituted C₃-C₁₀ carbocyclic ring or a substituted or unsubstituted C₃-C₁₀ heterocyclic ring, which may contain one or more heteroatoms and may be saturated or unsaturated; or R⁹ or R¹⁰ together with A₁ may form a substituted or unsubstituted C₄-C₁₀ fused carbocyclic ring or substituted or unsubstituted C₄-C₁₀ fused heterocyclic rings, which may contain one or more heteroatoms and may be saturated or unsaturated;
X is selected from the group consisting of 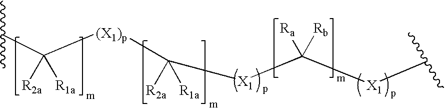
wherein each m is independently selected from an integer between 0 and 6; each p is independently selected from 0 or 1; each X₁ is independently selected from the group consisting of CR¹⁴R¹⁵, NR¹⁴, S, and O, —S(O), —S(O)₂, —OS(O)₂, —OS(O)₂O—, —C(O), C(OH), —C(O)O—, a substituted or unsubstituted aromatic, a substituted or unsubstituted heteroaromatic, and any combination thereof, in any orientation; each Rₐ and R_{b} are independently selected from the group consisting of hydrogen, cyano, and straight chain or branched C₁-C₆-alkyl, all of which may be optionally substituted with OH, halogen, straight chain or branched C₁-C₆-alkoxy, straight chain or branched halo-C₁-C₆-alkyl, straight chain and branched halo-C₁-C₆-alkoxy, C₁-C₆-alkoxy-C₁-C₆-alkyl, hydroxyl-C₁-C₆-alkyl, carboxy-C₁-C₆-alkyl, substituted or unsubstituted C₃-C₁₀ carbocyclic rings, and substituted or unsubstituted C₃-C₁₀ heterocyclic rings, which may contain one or more heteroatoms and may be saturated or unsaturated; or each Rₐ and R_{b} may form a 3-8-membered ring together with the carbon to which they are both attached;
each R₁ₐ and R₂ₐ are independently selected from the group consisting of hydrogen, halogen, cyano, and straight chain or branched C₁-C₆-alkyl, all of which may be optionally substituted with OH, halogen, straight chain or branched C₁-C₆-alkoxy, straight chain or branched halo-C₁-C₆-alkyl, straight chain and branched halo- C₁-C₆-alkoxy, C₁-C₆-alkoxy-C₁-C₆-alkyl, hydroxyl-C₁-C₆-alkyl, carboxy-C₁-C₆-alkyl, substituted or unsubstituted C₃-C₁₀ carbocyclic rings, and substituted or unsubstituted C₃-C₁₀ heterocyclic rings, which may contain one or more heteroatoms and may be saturated or unsaturated; or each R₁ₐ and R₂ₐ may form a 3-8-membered ring together with the carbon to which they are both attached; and
each R¹⁴ and R¹⁵ is independently selected from the group consisting of hydrogen, halogen, cyano, straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, and carboxy-C₁-C₆-alkyl; or each R¹⁴ or R¹⁵ may form a 3-8-membered ring together with B₁, SEM, Rₐ, R_{b}, R₁ₐ, or R₂ₐ and the atoms to which they are attached.

24. The compound of claim 23, wherein R¹ is L₁-O—H or L₁-O-L₂, wherein L₁ is a linking moiety and L₂ is a labile moiety.

25. The compound of claim 24, wherein R¹ is selected from the group consisting of -alkyl-OH, -halo-alkyl-OH, alkoxy-OH, -alkyl-OCOR⁴, -halo-alkyl-OCOR⁴, -alkoxy-OCOR⁴, -alkyl-OC(O)NR⁴R⁵, -halo-alkyl-OC(O)NHR⁴R⁵, -alkoxy-OC(O)NR⁴R⁵, —(CH₂)_{q}CO₂R⁶, and —(CH₂)ₙCH₂═CHC(O)OR⁶, wherein
q is an integer between 0 and 4;
R⁴and R⁵ are independently selected from the group consisting of hydrogen, straight chain or branched C₁-C₆-alkyl, all of which may be optionally substituted with OH, halogen, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl, substituted or unsubstituted C₃-C₁₀ carbocyclic rings, and substituted or unsubstituted C₃-C₁₀ heterocyclic rings, which may contain one or more heteroatoms and may be saturated or unsaturated; and
R is selected from the group consisting of hydrogen, straight chain or branched C₁-C₆-alkyl, straight chain or branched halo-C₁-C₆-alkyl, substituted or unsubstituted aryl group, and a prodrug derivatizing moiety (PDM).

26. The compound of claim 23, wherein R¹ is selected from the group consisting of —(CH₂)_{q}OPO₂R⁷R⁸, —(CH₂)_{q}OPO₃R⁷R⁸, and —(CH₂)_{q}OPO₂(S)R⁷R⁸, wherein
q is an integer between 0 and 4; and
R⁷ and R⁸ are each independently selected from the group consisting of hydrogen, straight chain or branched C₁-C₆-alkyl, straight chain or branched halo-C₁-C₆-alkyl, substituted or unsubstituted aryl group, and a prodrug derivatizing moiety (PDM).

27. The compound of claim 23, wherein R¹ is -L₁-Z₂, wherein L₁ is a linking moiety and Z₂ is a non-hydrolyzable moiety covalently bonded to L₁.

28. The compound of claim 27, wherein R¹ is selected from the group consisting of —(CH₂)_{q}CH₂PO₃R⁷R⁸, and —(CH₂)_{q}C(Y₁)(Y₂)PO₃R⁷R⁸, wherein
q is an integer between 0 and 4;
Y₁ and Y₂ are independently selected from the group consisting of hydrogen, straight chain or branched C₁-C₆-alkyl, all of which may be optionally substituted with OH, halogen, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl, substituted or unsubstituted C₃-C₁₀ carbocyclic rings, and substituted or unsubstituted C₃-C₁₀ heterocyclic rings, which may contain one or more heteroatoms and may be saturated or unsaturated; and
R⁷ and R⁸ are each independently selected from the group consisting of hydrogen, straight chain or branched C₁-C₆-alkyl, straight chain or branched halo-C₁-C₆-alkyl, substituted or unsubstituted aryl group, and a prodrug derivatizing moiety (PDM).

29. The compound of claim 28, wherein the PDM is selected from the group consisting of:

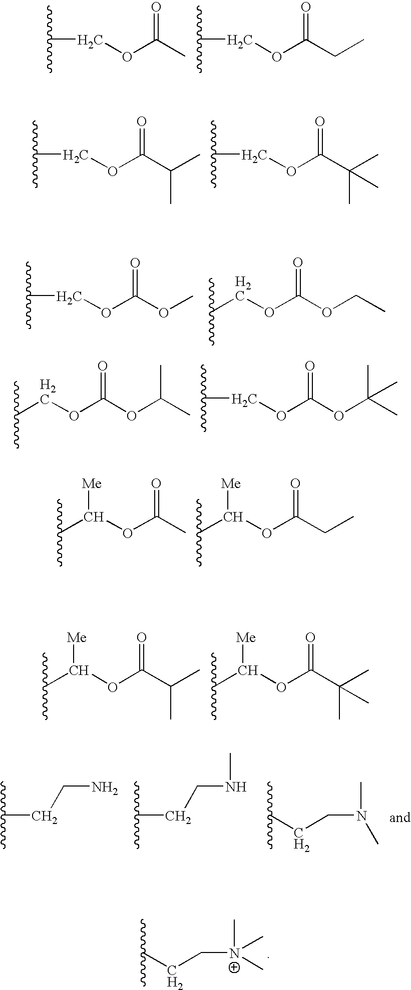

30. The compound of claim 23, wherein taken together R² and R³ form a substituted or unsubstituted C₃-C₁₀ carbocyclic ring or a substituted or unsubstituted C₁-C₁₀ heterocyclic ring, which may contain one or more heteroatoms and may be saturated or unsaturated, said ring contains at least one halogen.

31. The compound of claim 23, wherein A is selected from the group consisting of an aromatic ring and a heteroaromatic ring.

32. The compound of claim 23, wherein X is independently selected from the group consisting of straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl, C₁-C₆-alkyl-SO₂ alkylcarbonyl, thioether, alkylsulfonyl, alkylcarbonylamino, alkylaminocarbonyl, alkyloxycarbonyl, alkylcarbonyloxy, substituted or unsubstituted aromatic, and substituted or unsubstituted heteroaromatic; or taken together with either B₂ or C₁, R¹⁵ may form a substituted or unsubstituted C₃-C₁₀ carbocyclic ring or substituted or unsubstituted C₃-C₁₀ heterocyclic ring, which may contain one or more heteroatoms and may be saturated or unsaturated

33. The compound of claim 32, wherein X is selected from the group consisting of —CH₂NR¹⁴—, —CH₂NR¹⁴(CO)—, —CHFNR¹⁴—, —CHFNR¹⁴(CO)—, —CF₂NR¹⁴—, —CF₂NR¹⁴(CO)—, —CH₂(CO)—, —CHF(CO)—, —CF₂(CO)—, —(CO)CH₂—, —(CO)CHF—, —(CO)CF₂—, —CH₂(CHOH)—, —CHF(CHOH)—, —CF₂(CHOH)—, —(CHOH)CH₂—, —(CHOH) CHF—, —(CHOH)CF₂—, —NH(CO)—, —(CO)NH—, —(CO)—, —(CO)₂—, —O—, —S—, —SO—, —SO₂—, —CH₂O—, —CH₂CH₂O—, —CH₂OCH₂—, —OCH₂CH₂—, —OCH₂O—, —CH₂S—, —CH₂SO—, —CH₂SO₂—, —OCH₂—, —SCH₂—, —SOCH₂—, —SO₂CH₂—, —CHFO—, —CHFS—, —CHFSO—, —CHFSO₂—, —OCHF—, —SCHF—, —SOCHF—, —SO₂CHF—, —CF₂O—, —CF₂S—, —CF₂SO—, —CF₂SO₂—, —OCF₂—, —SCF₂—, —SOCF₂—, —SO₂CF₂—, —SO₂CF₂—, —NR¹⁴SO₂—, —SO₂NR¹⁴—, —CF₂—, —CF₂CF₂—, a aromatic group, and a heteroaromatic group.

34. The compound of claim 1 having the following formula: 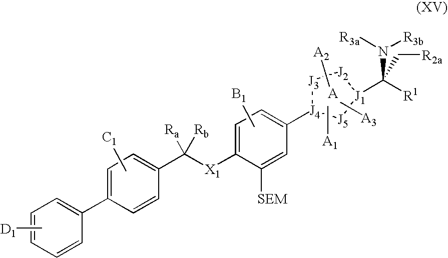
wherein:
the dashed lines represent a single or double bond;
SEM represents a selectivity enhancing moiety;
A₁, A₂, A₃, B₁, C₁, and D₁, are each independently selected from the group consisting of hydrogen, halogen, cyano, straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl, C₁-C₆-alkyl-SO₂ alkylcarbonyl, thioether, alkylsulfonyl, alkylcarbonylamino, alkylaminocarbonyl, alkyloxycarbonyl, alkylcarbonyloxy, substituted or unsubstituted aryl, substituted or unsubstituted heteroaryl, —OH, —CO₂-alkyl, —CO₂-halo-alkyl, —CONH₂, —CONH-alkyl, —CON-dialkyl, —CONH- halo-alkyl, —CON-halo-dialkyl, -alkyl-CONH-alkyl, -alkyl-CON-dialkyl, halo-alkyl-CONH-alkyl, halo-alkyl-CONH-halo-alkyl, alkyl-CONH-halo-dialkyl, —C₁-C₆-alkyl-hydroxyl, —C₁-C₆-alkyl-hydroxyl-alkyl, —C₁-C₆-halo-alkyl-hydroxyl-alkyl, —C₁-C₆-alkyl-hydroxyl-halo-alkyl, —C₁-C₆-halo-alkyl-hydroxyl-halo-alkyl, and N(R)R′; or taken together A₃ and B, may form a substituted or unsubstituted C₃-C₁₀ carbocyclic ring or substituted or unsubstituted C₃-C₁₀ heterocyclic ring, which may contain one or more heteroatoms and may be saturated or unsaturated; or taken together C₁ and D₁ may form a substituted or unsubstituted C₃-C₁₀ carbocyclic ring or substituted or unsubstituted C₃-C₁₀ heterocyclic ring, which may contain one or more heteroatoms and may be saturated or unsaturated;
R and R′ are each independently selected from the group consisting of hydrogen, halogen, cyano, straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, and carboxy-C₁-C₆-alkyl; or taken together R and R′ may form a substituted or unsubstituted C₃-C₁₀ carbocyclic ring or substituted or unsubstituted C₃-C₁₀ or heterocyclic ring, which may contain one or more heteroatoms and may be saturated or unsaturated; or taken together with the N to which they are attached R and R′ may form a moiety selected from the group consisting of substituted straight chain or cyclic guanyl, straight chain or cyclic guanidine, straight chain or cyclic urea, straight chain or cyclic thiourea, straight chain or cyclic carbamate, and straight chain or cyclic thiocarbamate;
J₁, J₂, J₃, J₄, and J₅ are independently selected from the group consisting of C, CH, N, NH, O, and S;
R¹ is a phosphate, a phosphate mimic or a phosphate precursor;
R₂ₐ is selected from the group consisting of hydrogen, halogen, cyano, straight chain or branched C₁-C₆-alkyl, and straight chain or branched halo-C₁-C₆-alkyl, all of which may be optionally substituted with OH, halogen, —OR⁹, or —OC(O)R⁹;
R₃ₐ and R_{3b} are each independently selected from the group consisting of hydrogen, halogen, cyano, straight chain or branched C₁-C₆-alkyl, straight chain or branched halo-C₁-C₆-alkyl; or taken together R₃ₐ and R_{3b} may form a group selected from the group consisting of C₃-C₆-carbocycle and C₃-C₆-halo-carbocycle;
R⁹ is selected from the group consisting of hydrogen, halogen, cyano, straight chain or branched C₁-C₆-alkyl, straight chain or branched C₁-C₆-alkoxy, straight chain or branched halo-C₁-C₆-alkyl, straight chain and branched halo-C₁-C₆-alkoxy, substituted or unsubstituted C₃-C₁₀ carbocyclic rings, and substituted or unsubstituted C₃-C₁₀ heterocyclic rings, which may contain one or more heteroatoms and may be saturated or unsaturated;
X₁ is selected from the group consisting of CR¹⁴R¹⁵, NR¹⁴, S, and O, —S(O), —S(O)₂, —OS(O)₂, —OS(O)₂O—, —C(O), C(OH), —C(O)O—, any five- or six-membered aromatic or heteroaromatic, isomers and tautomers thereof, and any combination thereof, in any orientation;
each Rₐ and R_{b} are each independently selected from the group consisting of hydrogen, halogen, alkyl, halo-alkyl, —OH, —CO—, straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, —C₁-C₆-alkyl-hydroxyl, —C₁-C₆-alkyl-hydroxyl-alkyl, —C₁-C₆-halo-alkyl-hydroxyl-alkyl, —C₁-C₆-alkyl-hydroxyl-halo-alkyl, —C₁-C₆-halo-alkyl-hydroxyl-halo-alkyl, carboxy-C₁-C₆-alkyl, C₁-C₆-alkyl-SO₂, alkylcarbonyl, thioether, alkylsulfonyl, alkylcarbonylamino, alkylaminocarbonyl, alkyloxycarbonyl, alkylcarbonyloxy, substituted or unsubstituted aryl, and substituted or unsubstituted heteroaryl; or taken together each Rₐ and R_{b} may form a C₃-C₆-carbocycle, C₁-C₆-halo-carbocycle, substituted or unsubstituted C₃-C₁₀ carbocyclic rings and substituted or unsubstituted C₃-C₁₀clic rings, which may contain one or more heteroatoms and may be saturated or unsaturated;
each R¹⁴ and R¹⁵ is independently selected from the group consisting of hydrogen, halogen, cyano, straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, and carboxy-C₁-C₆-alkyl; or each R¹⁴ or R¹⁵ may form a 3-8-membered ring together with B₁, Rₐ, or R_{b}, and the atoms to which they are attached.

35. The compound of claim 34, wherein R′ is L₁-O—H or L₁-O-L₂, wherein L₁ is a linking moiety and L₂ is a labile moiety.

36. The compound of claim 35, wherein R¹ is selected from the group consisting of -alkyl-OH, -halo-alkyl-OH, alkoxy-OH, -alkyl-OCOR⁴, -halo-alkyl-OCOR⁴, -alkoxy-OCOR⁴, -alkyl-OC(O)NR⁴R⁵, -halo-alkyl-OC(O)NHR⁴R⁵, -alkoxy-OC(O)NR⁴R⁵, —(CH₂)_{q}CO₂R⁶, and —(CH₂)ₙCH₂═CHC(O)OR⁶, wherein
q is an integer between 0 and 4;
R⁴and R⁵ are independently selected from the group consisting of hydrogen, straight chain or branched C₁-C₆-alkyl, all of which may be optionally substituted with OH, halogen, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl, substituted or unsubstituted C₃-C₁₀ carbocyclic rings, and substituted or unsubstituted C₃-C₁₀ heterocyclic rings, which may contain one or more heteroatoms and may be saturated or unsaturated; and
R⁶ is selected from the group consisting of hydrogen, straight chain or branched C₁-C₆-alkyl, straight chain or branched halo-C₁-C₆-alkyl, substituted or unsubstituted aryl group, and a prodrug derivatizing moiety (PDM).

37. The compound of claim 34, wherein R¹ is selected from the group consisting of —(CH₂)_{q}OPO₂R⁷R⁸, —(CH₂)_{q}OPO₃R⁷R⁸, and —(CH₂)_{q}OPO₂(S)R⁷R⁸, wherein
q is an integer between 0 and 4; and
R⁷ and R⁸ are each independently selected from the group consisting of hydrogen, straight chain or branched C₁-C₆-alkyl, straight chain or branched halo-C₁-C₆-alkyl, substituted or unsubstituted aryl group, and a prodrug derivatizing moiety (PDM).

38. The compound of claim 34, wherein R¹ is -L₁-Z₂, wherein L₁ is a linking moiety and Z₂ is a non-hydrolyzable moiety covalently bonded to L₁.

39. The compound of claim 38, wherein R¹ is selected from the group consisting of —(CH₂)_{q}CH₂PO₃R⁷R⁸, and —(CH₂)_{q}C(Y₁)(Y₂)PO₃R⁷R⁸, wherein
q is an integer between 0 and 4;
Y₁ and Y₂ are independently selected from the group consisting of hydrogen, straight chain or branched C₁-C₆-alkyl, all of which may be optionally substituted with OH, halogen, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl, substituted or unsubstituted C₃-C₁₀ carbocyclic rings, and substituted or unsubstituted C₃-C₁₀ heterocyclic rings, which may contain one or more heteroatoms and may be saturated or unsaturated; and
R⁷ and R⁸ are each independently selected from the group consisting of hydrogen, straight chain or branched C₁-C₆-alkyl, straight chain or branched halo-C₁-C₆-alkyl, substituted or unsubstituted aryl group, and a prodrug derivatizing moiety (PDM).

40. The compound of claim 39, wherein the PDM is selected from the group consisting of:

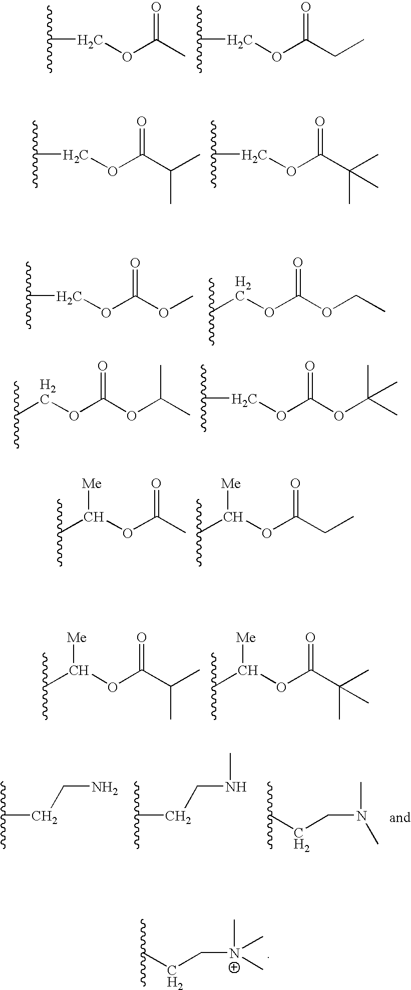

41. The compound of claim 34, wherein A is selected from the group consisting of a 5-membered aromatic ring and a 5-membered heteroaromatic ring.

42. The compound of claim 1 having the following formula: 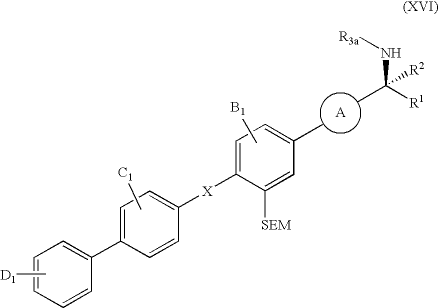
wherein:
SEM represents a selectivity enhancing moiety;
ring A is selected from the group consisting of any five- or six-membered aromatic or heteroaromatic, and isomers and tautomers thereof;
R′ is a phosphate, a phosphate mimic or a phosphate precursor;
R₂ is selected from the group consisting of —H, —F, —CN, —OH, —CH₂OH, —CHFOH, CF₂OH, CH(CH₃)OH, CF(CH₃)OH, CH(CF₃)OH, —CH₃, —CH₂CH₃, —CF₃, —CF₂CF₃, cyclopropyl, fluorinated cyclopropyl, —CH₂OR⁹, —CH₂OC(O)R⁹,
R⁹ is selected from a group consisting of straight chain or branched C₁-C₆-alkyl, straight chain or branched C₁-C₆-alkoxy, straight chain or branched halo-C₁-C₆-alkyl, straight chain and branched halo-C₁-C₆-alkoxy, substituted or unsubstituted C₃-C₁₀ carbocyclic rings, and substituted or unsubstituted C₃-C₁₀ heterocyclic rings, which may contain one or more heteroatoms and may be saturated or unsaturated;
X is selected from the group consisting of 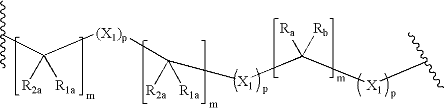
wherein each m is independently selected from an integer between 0 and 6; each p is independently selected from 0 or 1; each X₁ is independently selected from the group consisting of CR⁴R⁵, NR¹⁴, S, and O, —S(O), —S(O)₂, —OS(O)₂, —OS(O)₂O—, —C(O), C(OH), —C(O)O—, a substituted or unsubstituted aromatic, a substituted or unsubstituted heteroaromatic, and any combination thereof, in any orientation; each Rₐ and R_{b} are independently selected from the group consisting of hydrogen, cyano, halogen, alkyl, halo-alkyl, —OH, —CO—, straight chain or branched alkoxy, straight chain or branched halo-alkyl, straight chain or branched halo-alkoxy, alkoxy-alkyl, hydroxyl-alkyl, -alkyl-hydroxyl, -alkyl-hydroxyl-alkyl, -halo-alkyl-hydroxyl-alkyl, -alkyl-hydroxyl-halo-alkyl, -halo-alkyl-hydroxyl-halo-alkyl, carboxy-alkyl, alkyl-SO₂, alkylcarbonyl, thioether, alkylsulfonyl, alkylcarbonylamino, alkylaminocarbonyl, alkyloxycarbonyl, alkylcarbonyloxy, substituted or unsubstituted aryl, and substituted or unsubstituted heteroaryl, all of which may be optionally substituted with OH, halogen, straight chain or branched alkoxy, straight chain or branched halo-alkyl, straight chain and branched halo-alkoxy, alkoxy-alkyl, hydroxyl-alkyl, carboxy-alkyl, substituted or unsubstituted carbocyclic rings, and substituted or unsubstituted heterocyclic rings, which may contain one or more heteroatoms and may be saturated or unsaturated; or each Rₐ and R_{b} may form a 3-10-membered ring together with the carbon to which they are both attached;
each R₁ₐ and R₂ₐ are independently selected from the group consisting of hydrogen, cyano, halogen, alkyl, halo-alkyl, —OH, —CO—, straight chain or branched alkoxy, straight chain or branched halo-alkyl, straight chain or branched halo-alkoxy, alkoxy-alkyl, hydroxyl-alkyl, -alkyl-hydroxyl, -alkyl-hydroxyl-alkyl, -halo-alkyl-hydroxyl-alkyl, -alkyl-hydroxyl-halo-alkyl, -halo-alkyl-hydroxyl-halo-alkyl, carboxy-alkyl, alkyl-SO₂, alkylcarbonyl, thioether, alkylsulfonyl, alkylcarbonylamino, alkylaminocarbonyl, alkyloxycarbonyl, alkylcarbonyloxy, substituted or unsubstituted aryl, and substituted or unsubstituted heteroaryl, all of which may be optionally substituted with OH, halogen, straight chain or branched alkoxy, straight chain or branched halo-alkyl, straight chain and branched halo-alkoxy, alkoxy-alkyl, hydroxyl-alkyl, carboxy-alkyl, substituted or unsubstituted carbocyclic rings, and substituted or unsubstituted heterocyclic rings, which may contain one or more heteroatoms and may be saturated or unsaturated; or each R₁ₐ and R₂ₐ may form a 3-10-membered ring together with the carbon to which they are both attached; and
each R¹⁴ and R¹⁵ is independently selected from the group consisting of hydrogen, halogen, cyano, straight chain or branched alkyl, straight chain or branched alkoxy, straight chain or branched halo-alkyl, straight chain or branched halo-alkoxy, alkoxy-alkyl, hydroxyl-alkyl, alkyl-SO₂, and carboxy-alkyl; or each R¹⁴ or R¹⁵ may form a 3-8-membered ring together with B₁, SEM, Rₐ, R_{b}, R₁ₐ, or R₂ₐ and the atoms to which they are attached.
R₃ₐ is selected from the group consisting of consisting of —H, straight chain or branched C₁-C₆-alkyl, straight chain or branched halo-C₁-C₆-alkyl, substituted or unsubstituted C₃-C₁₀ carbocyclic rings and substituted or unsubstituted C₃-C₁₀ heterocyclic rings, —C(O)alkyl, —C(O)NH-alkyl, —C(O)N-dialkyl, —C(O)aryl, —C(O)NH-aryl, —C(O)N-alkyl-aryl, —C(O)N-diaryl, —C(O)heteroaryl, —C(O)NH-heteroaryl, —C(O)N-carbocycle;
D₁, C₁, and B₁ are each independently selected from the group consisting of —H, —F, —Cl, —Br, —I, -alkyl, -halo-alkyl, —CN, —COR , —CH₂OR , —CHFOR , CF₂OR¹⁶, —OR¹⁶, alkylcarbonyl, thioether, alkylsulfonyl, alkylcarbonylamino, alkylaminocarbonyl, alkyloxycarbonyl, alkylcarbonyloxy, substituted or unsubstituted aromatic , substituted or unsubstituted heteroaromatic, straight chain or branched alkylene, straight chain or branched alkenyl, straight chain or branched alkynyl, straight chain or branched alkenylene, arylalkyl, alkylaryl, alkylene-aryl, alkenyl-aryl, alkynyl-aryl, and alkenylene-aryl groups, and —N(R¹⁶)R¹⁷, aryl; and
R¹⁶ and R¹⁷ are each independently selected from the group consisting of hydrogen, straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl, and C₁-C₆-alkyl-SO₂.

43. The compound of claim 42, wherein R¹ is L₁-O—H or L₁-O-L₂, wherein L₁ is a linking moiety and L₂ is a labile moiety.

44. The compound of claim 43, wherein R¹ is selected from the group consisting of -alkyl-OH, -halo-alkyl-OH, alkoxy-OH, -alkyl-OCOR⁴, -halo-alkyl-OCOR⁴, -alkoxy-OCOR⁴, -alkyl-OC(O)NR⁴R⁵, -halo-alkyl-OC(O)NHR⁴R⁵, -alkoxy-OC(O)NR⁴R⁵, —(CH₂)_{q}CO₂R⁶, and —(CH₂)ₙCH₂═CHC(O)OR⁶, wherein
q is an integer between 0 and 4;
R⁴and R⁵ are independently selected from the group consisting of hydrogen, straight chain or branched C₁-C₆-alkyl, all of which may be optionally substituted with OH, halogen, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl, substituted or unsubstituted C₃-C₁₀ carbocyclic rings, and substituted or unsubstituted C₃-C₁₀ heterocyclic rings, which may contain one or more heteroatoms and may be saturated or unsaturated; and
R⁶ is selected from the group consisting of hydrogen, straight chain or branched C₁-C₆-alkyl, straight chain or branched halo-C₁-C₆-alkyl, substituted or unsubstituted aryl group, and a prodrug derivatizing moiety (PDM).

45. The compound of claim 42, wherein R₁ is selected from the group consisting of —(CH₂)_{q}OPO₂R⁷R⁸, —(CH₂)_{q}OPO₃R⁷R⁸, and —(CH₂)_{q}OPO₂(S)R⁷R⁸, wherein
q is an integer between 0 and 4; and
R⁷ and R⁸ are each independently selected from the group consisting of hydrogen, straight chain or branched C₁-C₆-alkyl, straight chain or branched halo-C₁-C₆-alkyl, substituted or unsubstituted aryl group, and a prodrug derivatizing moiety (PDM).

46. The compound of claim 42, wherein R¹ is -L₁-Z₂, wherein L₁ is a linking moiety and Z₂ is a non-hydrolyzable moiety covalently bonded to L₁.

47. The compound of claim 46, wherein. R¹ is selected from the group consisting of —(CH₂)_{q}CH₂PO₃R⁷R⁸, and —(CH₂)_{q}C(Y₁)(Y₂)PO₃R⁷R⁸, wherein
q is an integer between 0 and 4;
Y₁ and Y₂ are independently selected from the group consisting of hydrogen, straight chain or branched C₁-C₆-alkyl, all of which may be optionally substituted with OH, halogen, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl, substituted or unsubstituted C₃-C₁₀ carbocyclic rings, and substituted or unsubstituted C₃-C₁₀ heterocyclic rings, which may contain one or more heteroatoms and may be saturated or unsaturated; and
R⁷ and R⁸ are each independently selected from the group consisting of hydrogen, straight chain or branched C₁-C₆-alkyl, straight chain or branched halo-C₁-C₆-alkyl, substituted or unsubstituted aryl group, and a prodrug derivatizing moiety (PDM).

48. The compound of claim 42, wherein the SEM is selected from the group consisting of —F, —Cl, —Br, —I, -halo-alkyl, —CN, —COR¹⁸, —CH₂OR¹⁸, —CHFOR¹⁸, CF₂OR¹⁸, —OR, —N(R¹⁸)R¹⁹, aryl, alkylcarbonyl, thioether, alkylsulfonyl, alkylcarbonylamino, alkylaminocarbonyl, alkyloxycarbonyl, alkylcarbonyloxy, substituted or unsubstituted aromatic, substituted or unsubstituted heteroaromatic, straight chain or branched alkyl, straight chain or branched alkenyl, straight chain or branched alkynyl, straight chain or branched alkenyl, arylalkyl, alkylaryl, alkenyl-aryl, and alkynyl-aryl, groups;
wherein R¹⁸ and R¹⁹ are each independently selected from hydrogen, straight chain or branched C₁-C₆-alkyl, straight chain or branched C₁-C₆-alkoxy, straight chain or branched halo-C₁-C₆-alkyl, straight chain or branched halo-C₁-C₆-alkoxy, C₁-C₆-alkoxy-C₁-C₆-alkyl, hydroxyl-C₁-C₆-alkyl, carboxy-C₁-C₆-alkyl or C₁-C₆-alkyl-SO₂.

49. The compound of claim 48, wherein the PDM is selected from the group consisting of:

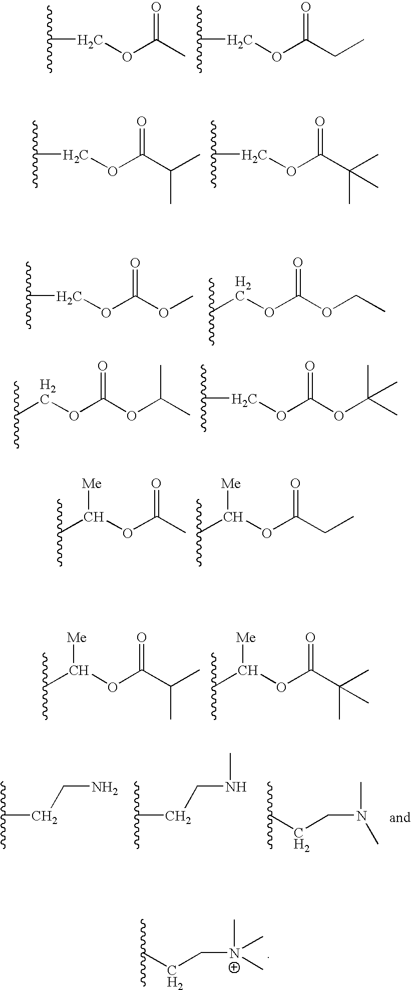

50. The compound of claim 42, wherein A is selected from the group consisting of an aromatic ring and a heteroaromatic ring.

51. The compound of claim 50, wherein ring A is selected from the group consisting of

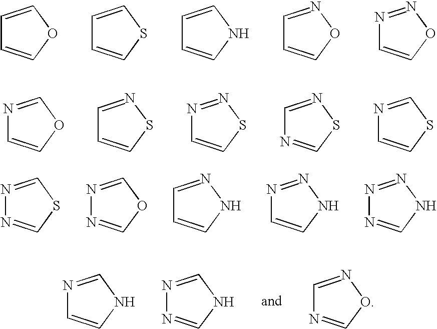

52. The compound of claim 42 having the following formula: 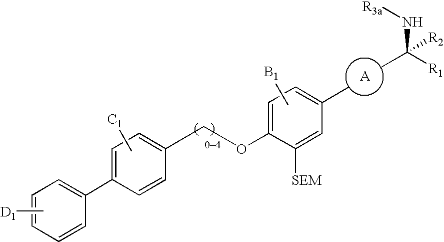
wherein SEM, B₁, C₁, D₁, R₁, R₂, and R₃ₐ are as defined in claim 42.

53. The compound of claim 1 selected from the group consisting of

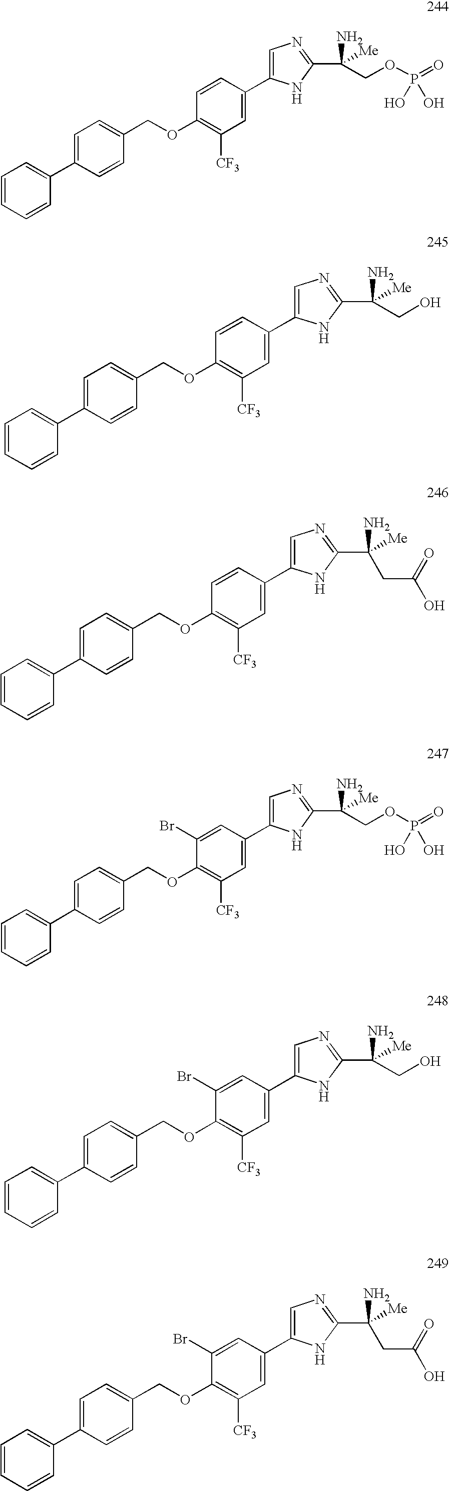

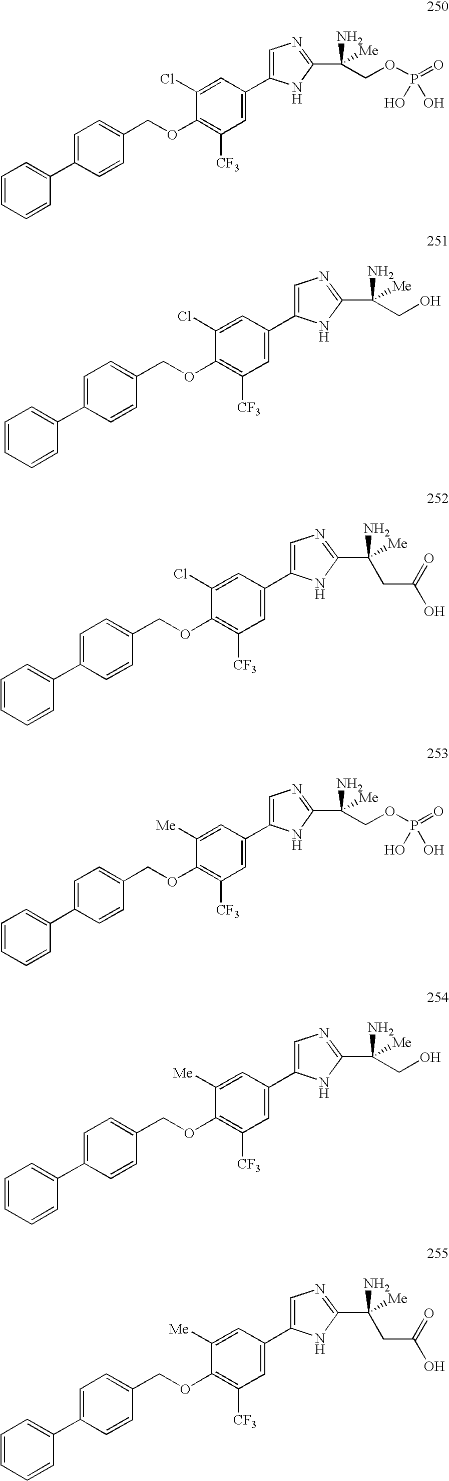

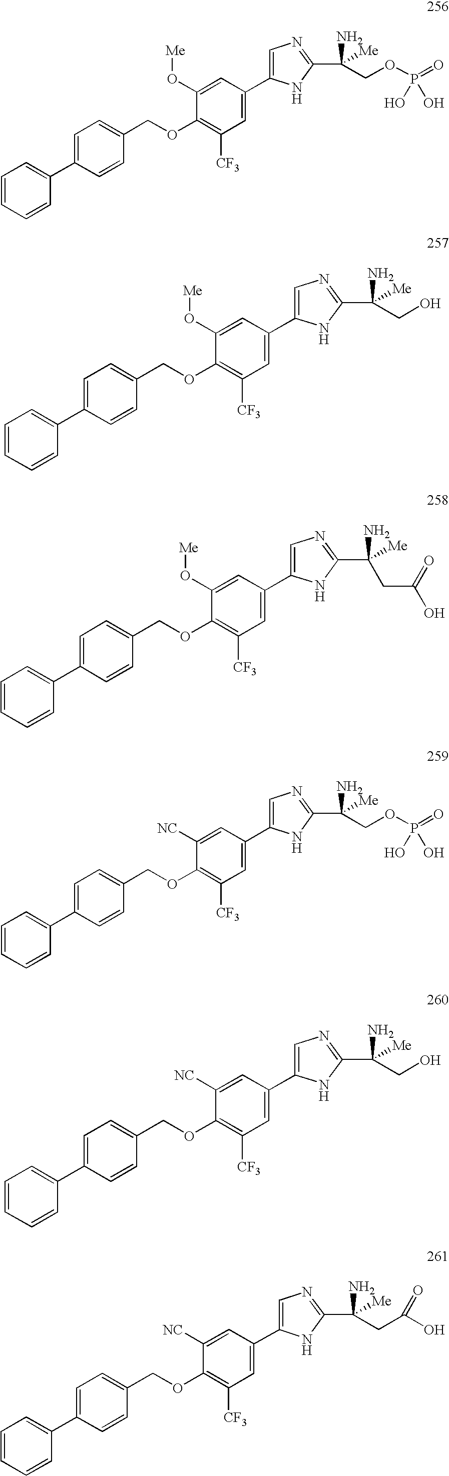

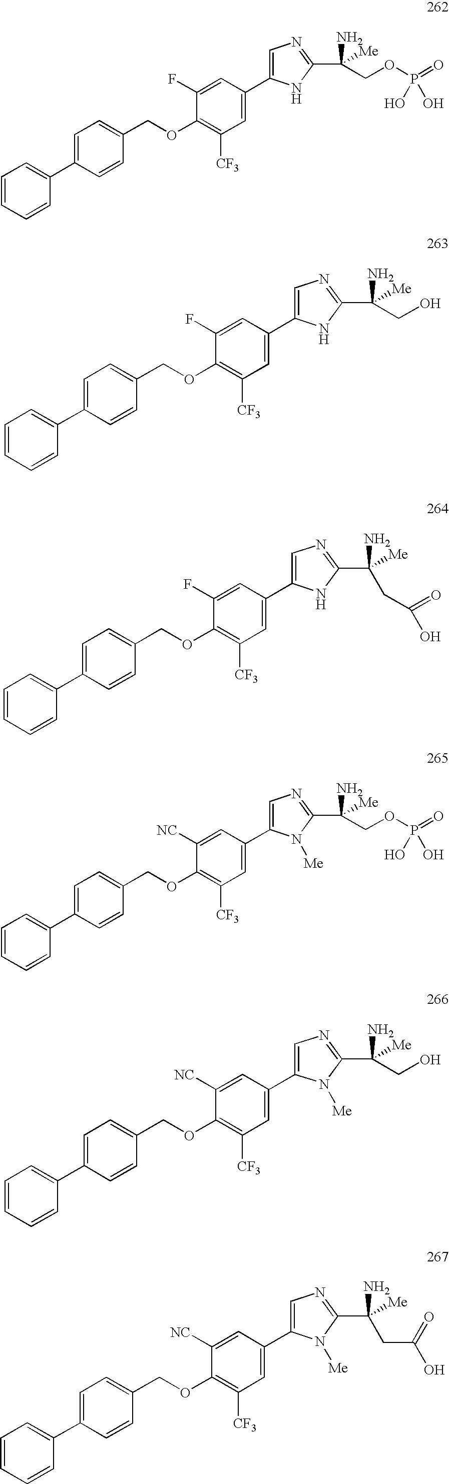

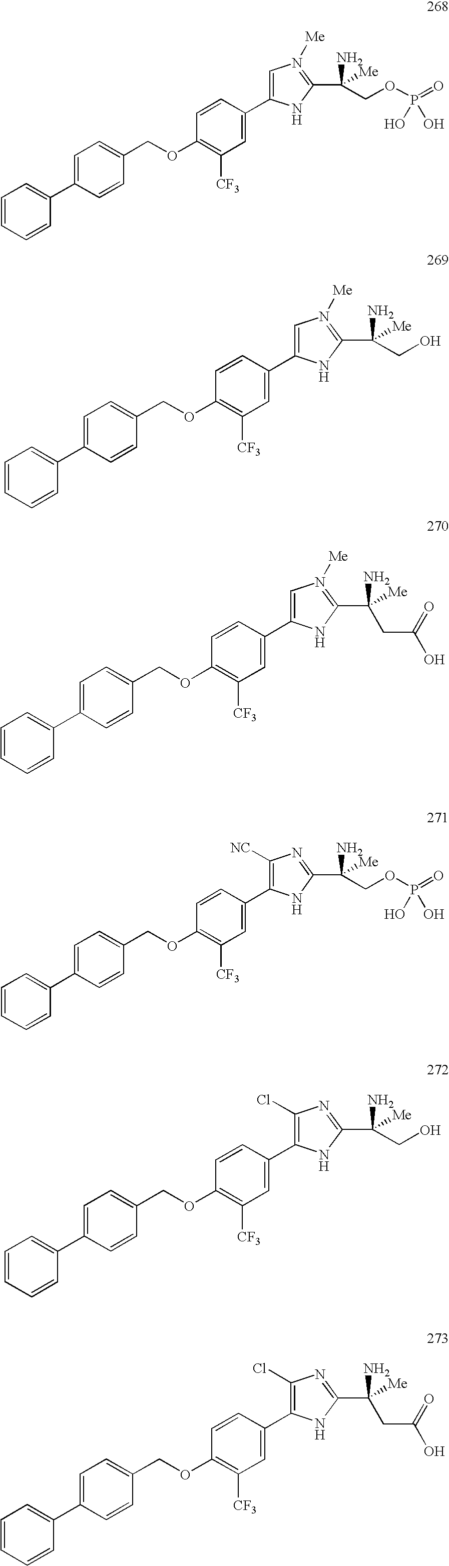

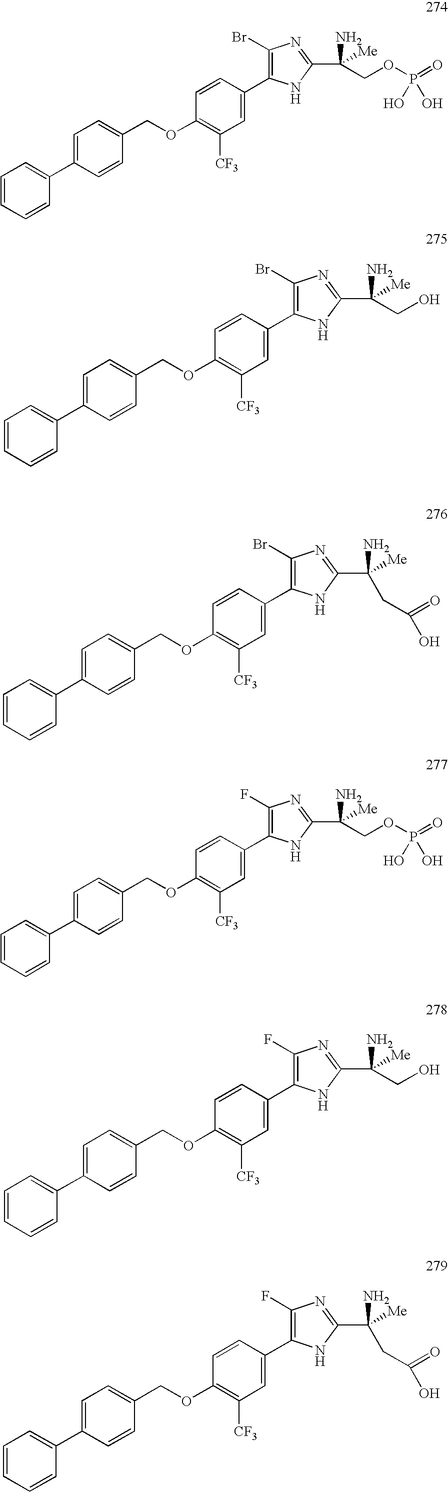

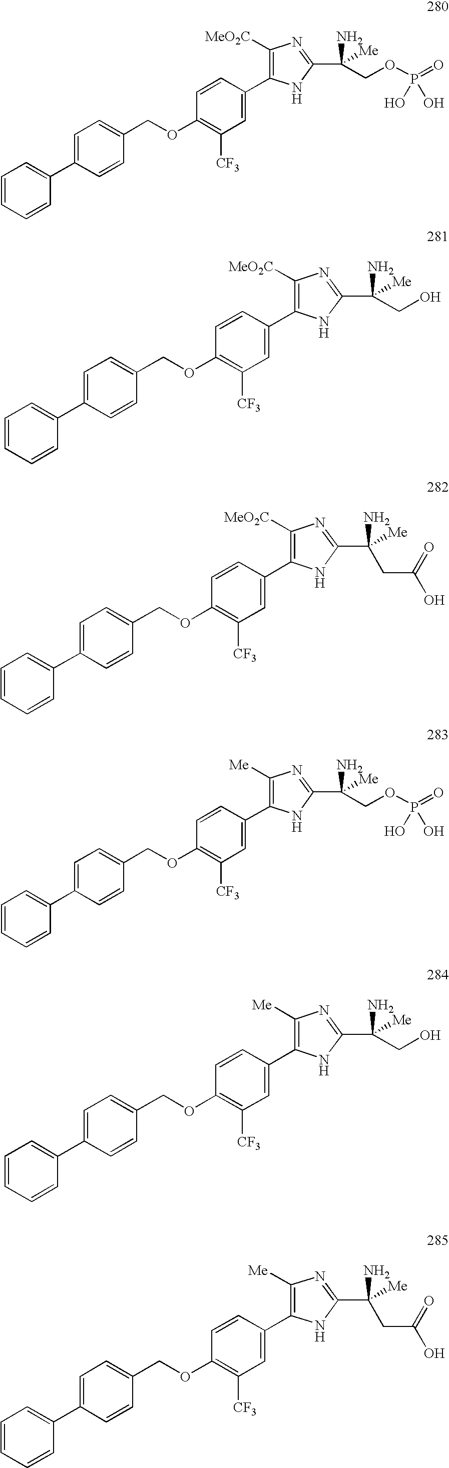

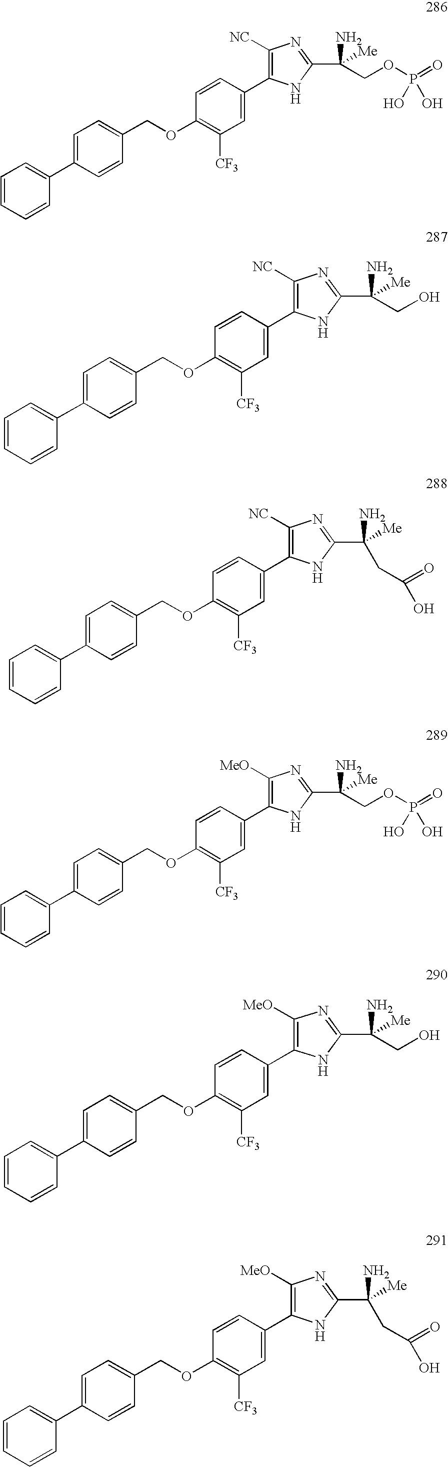

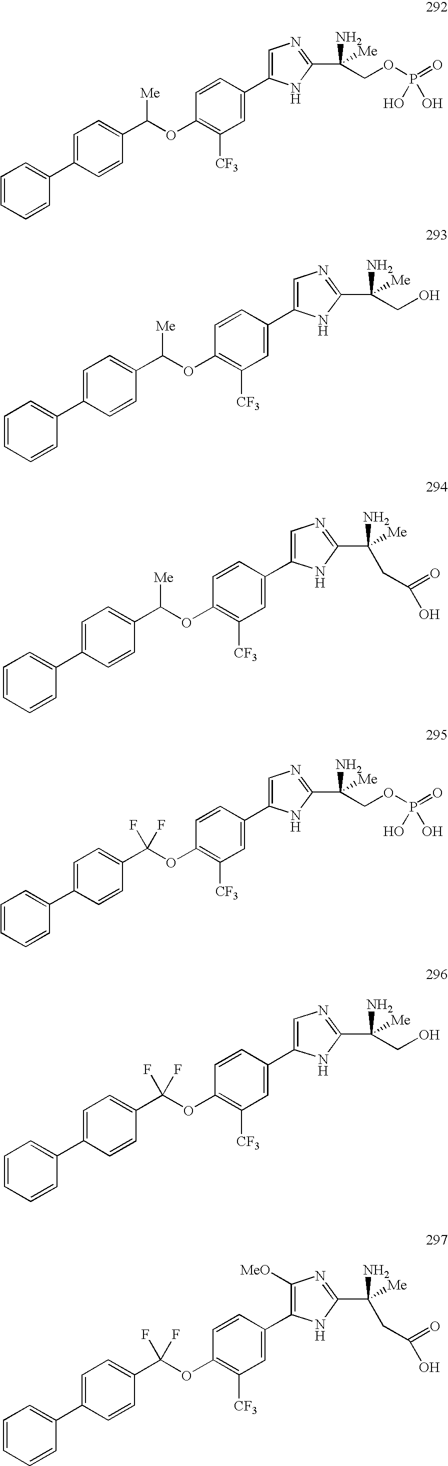

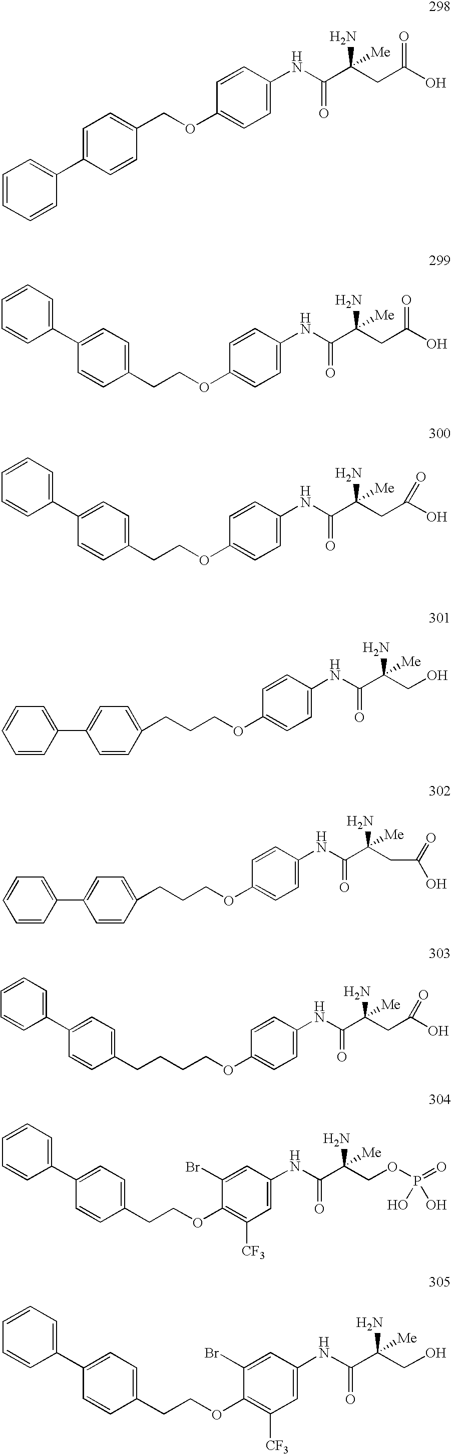

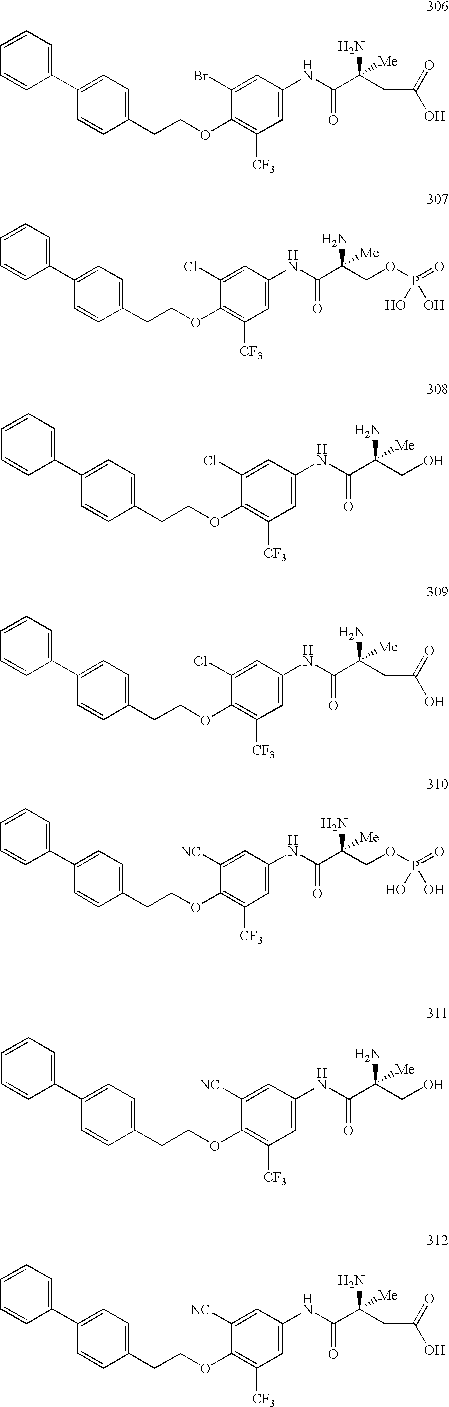

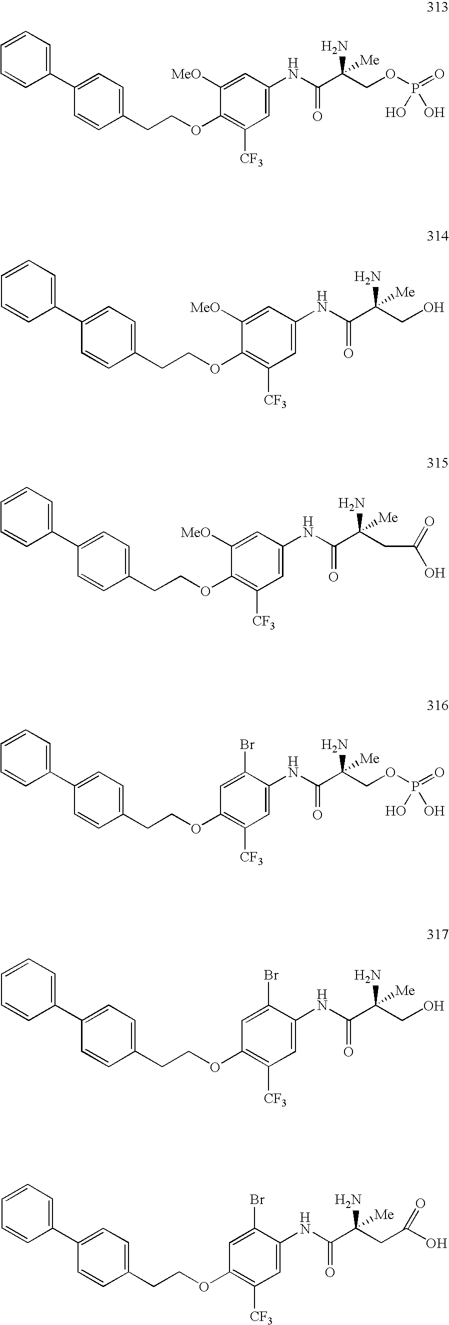

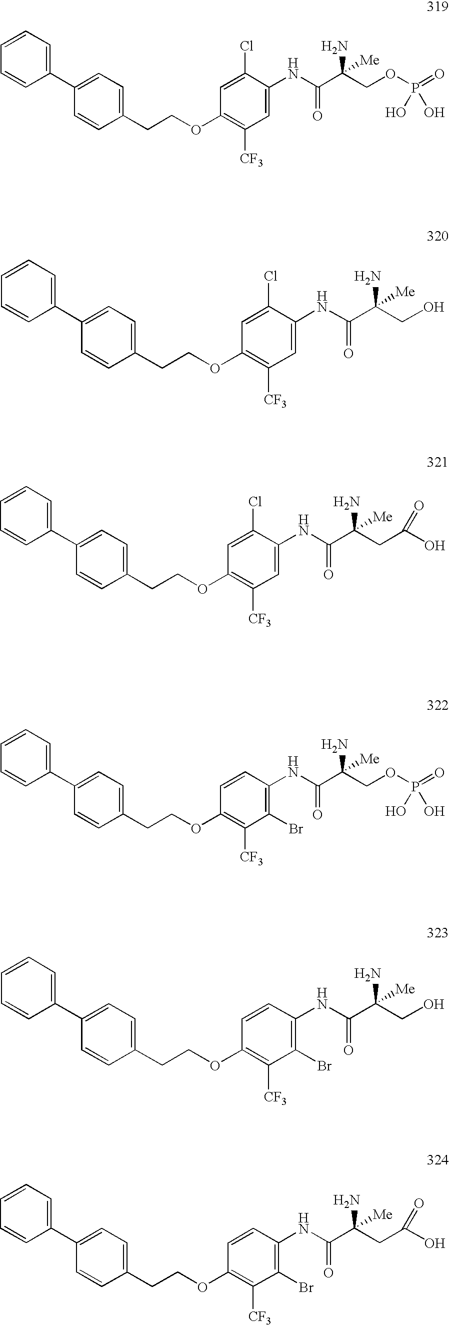

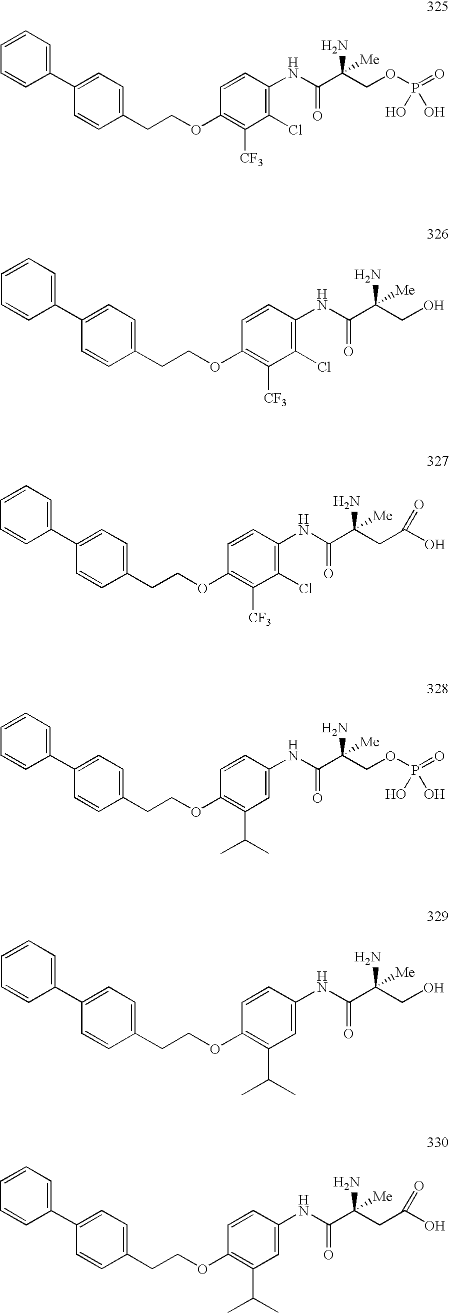

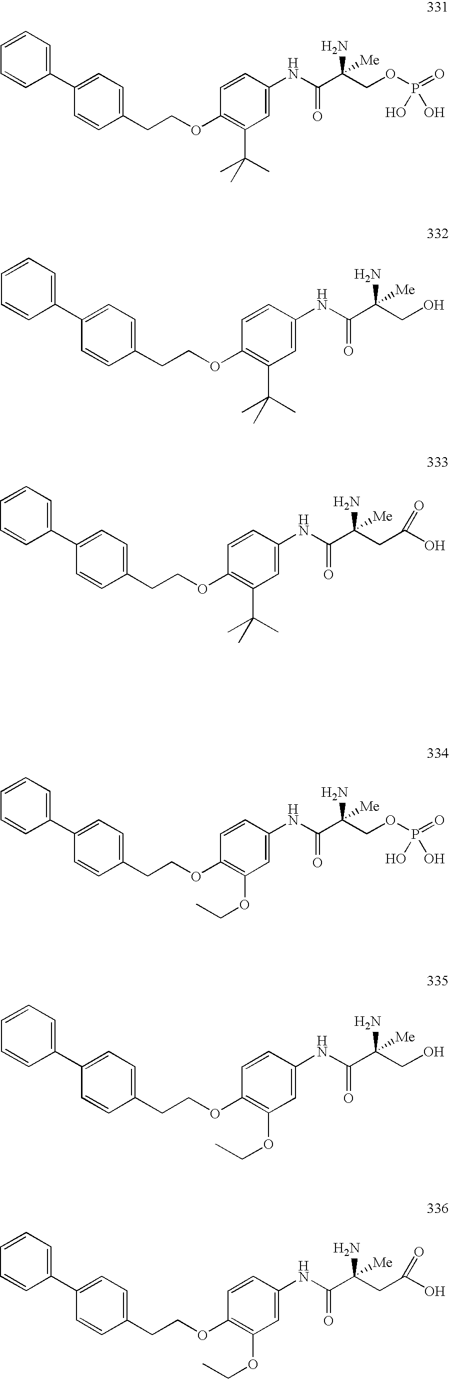

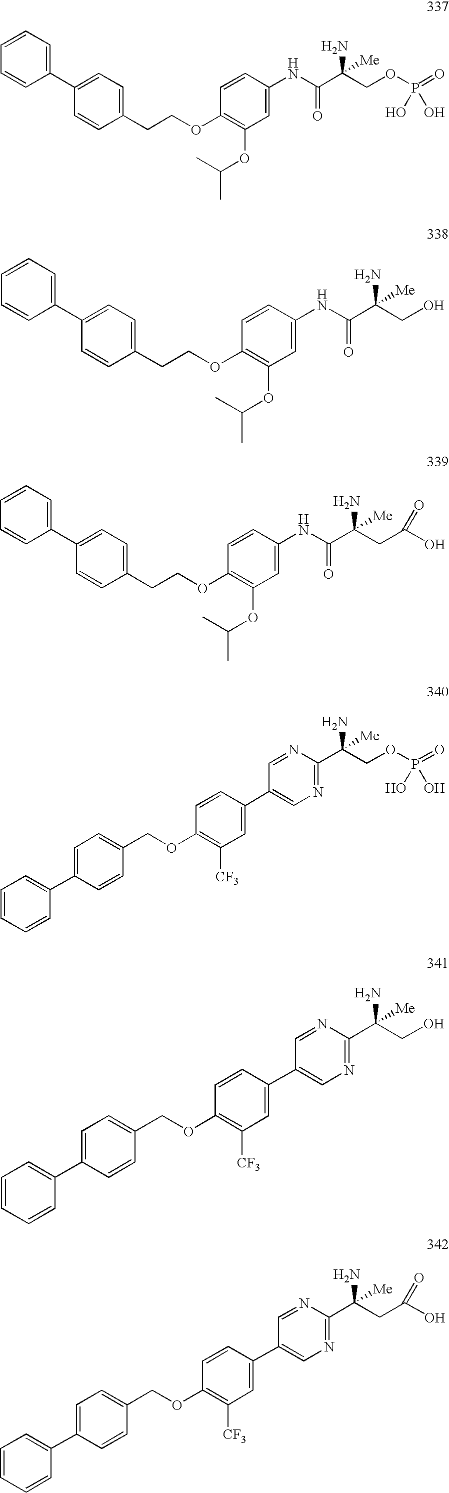

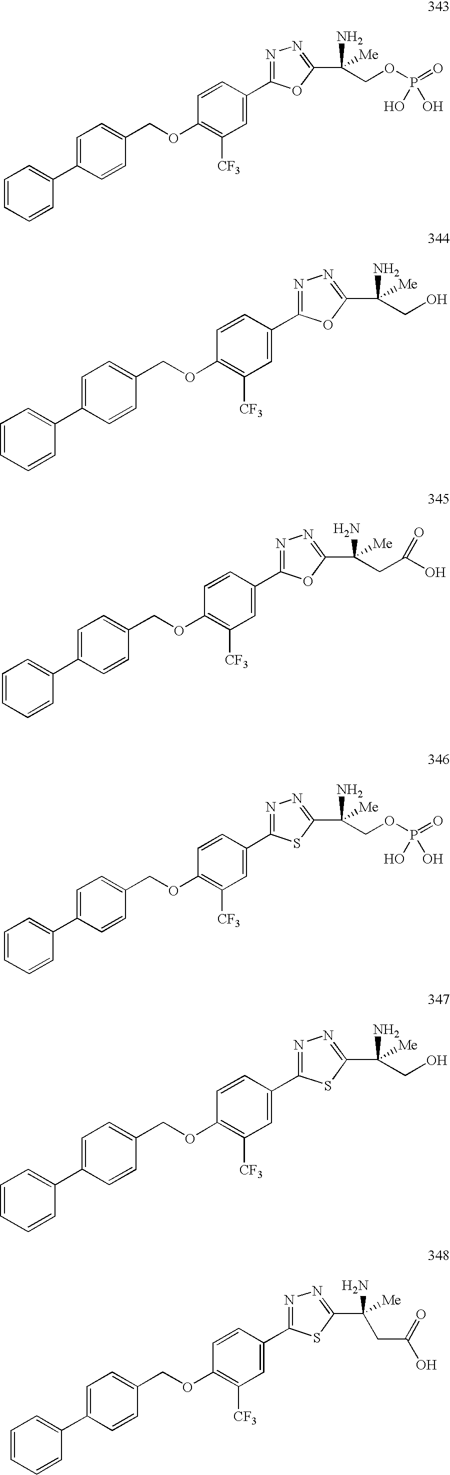

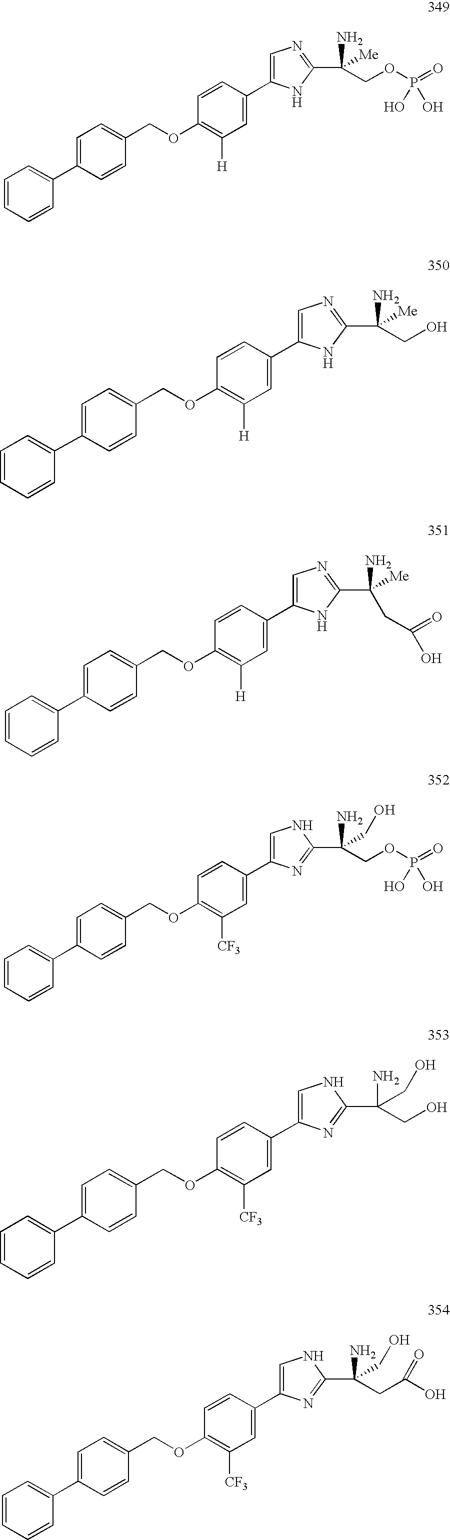

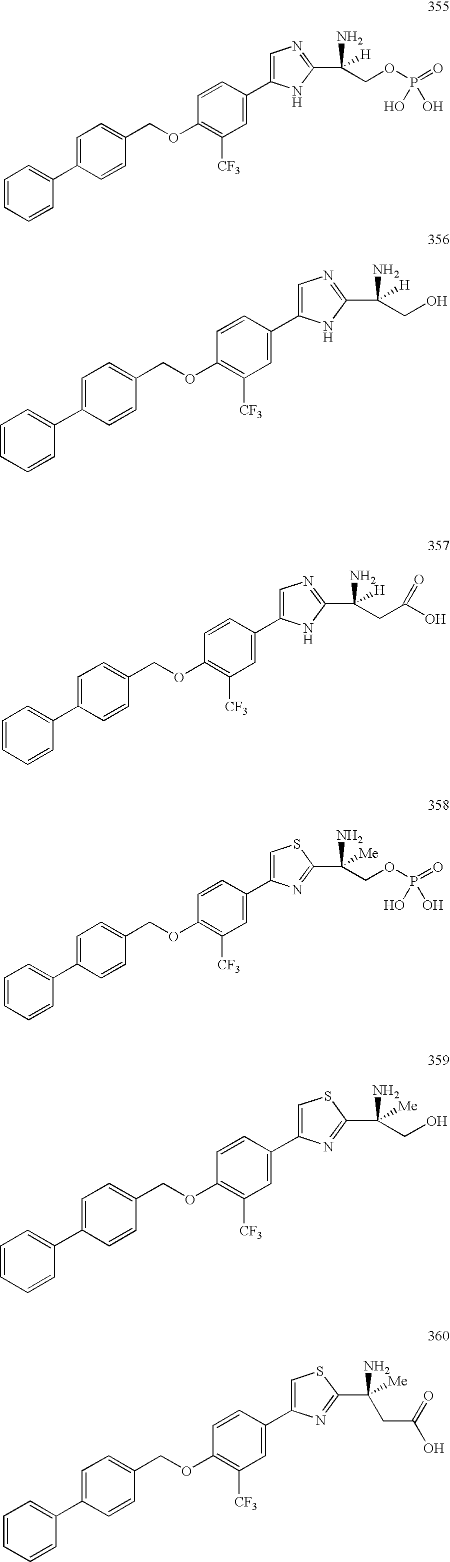

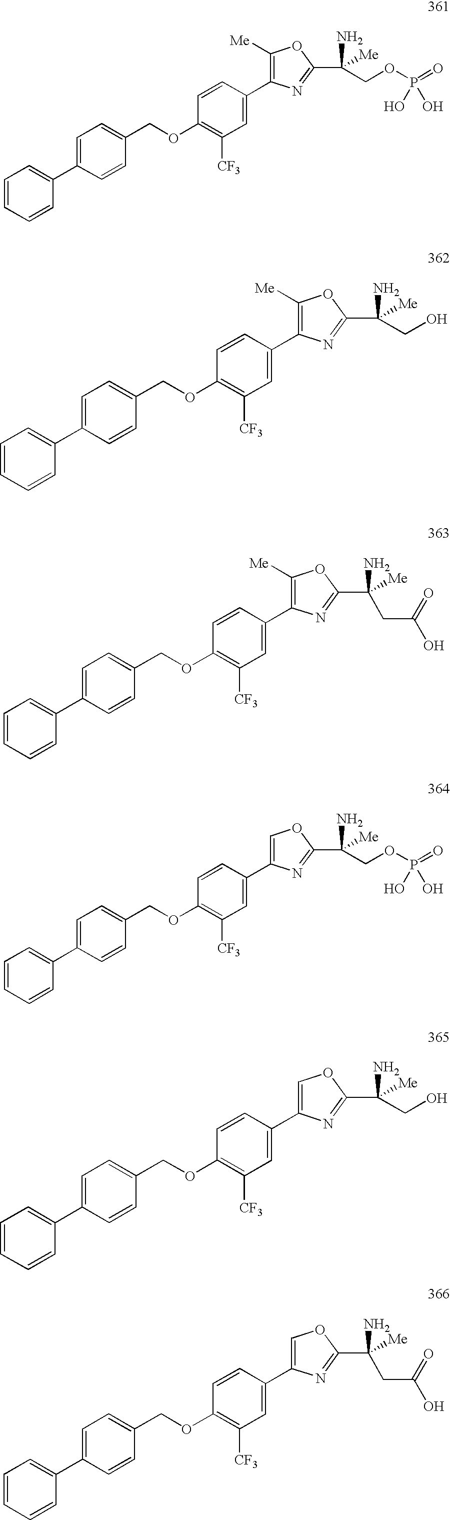

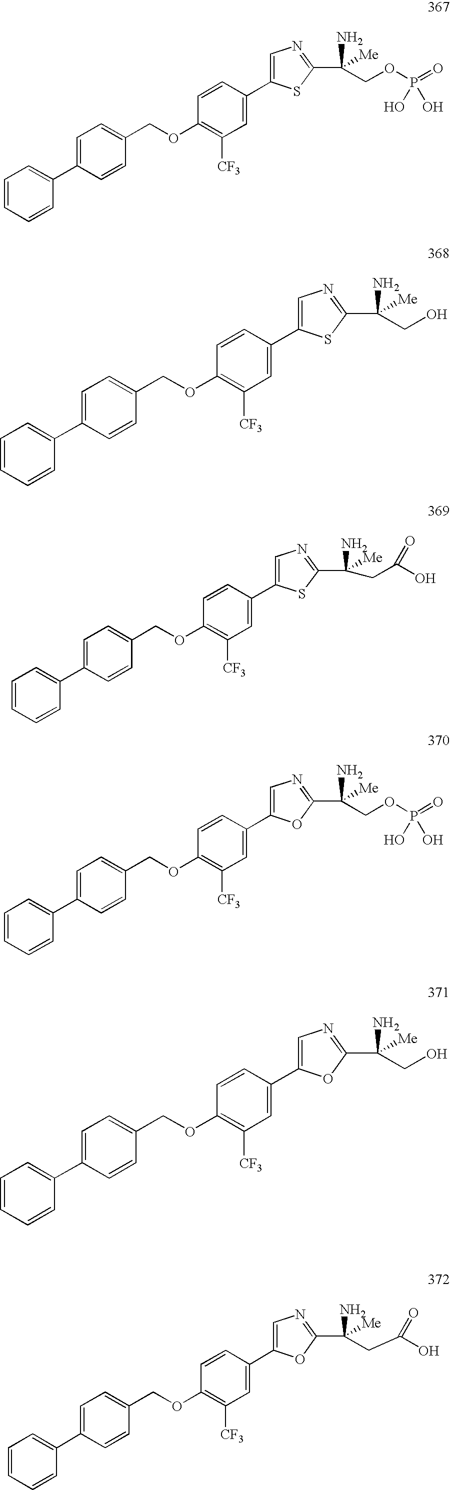

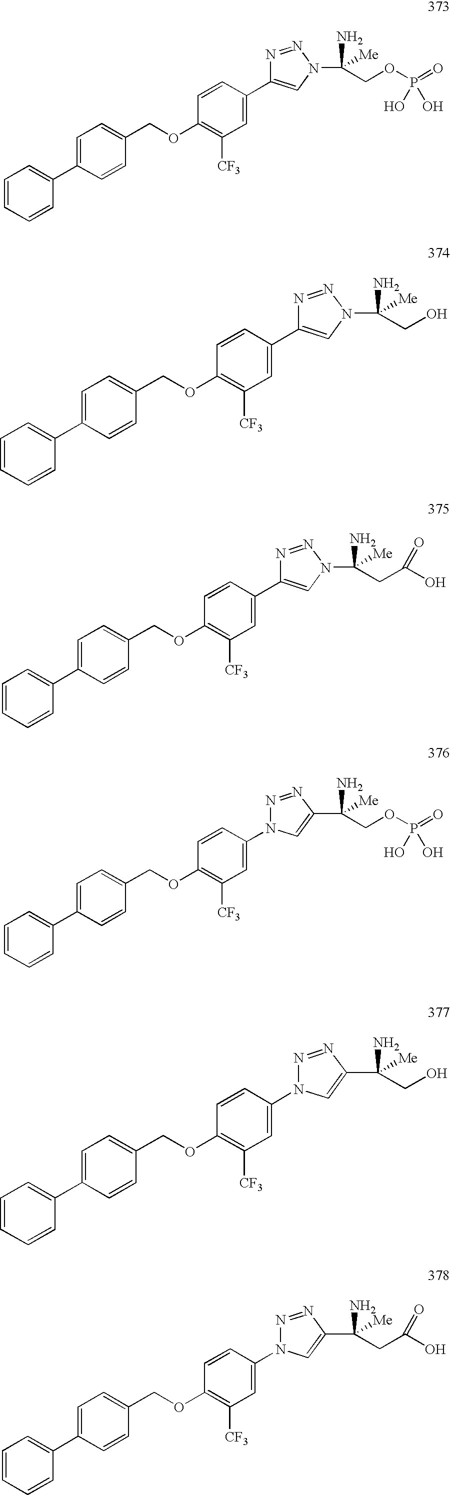

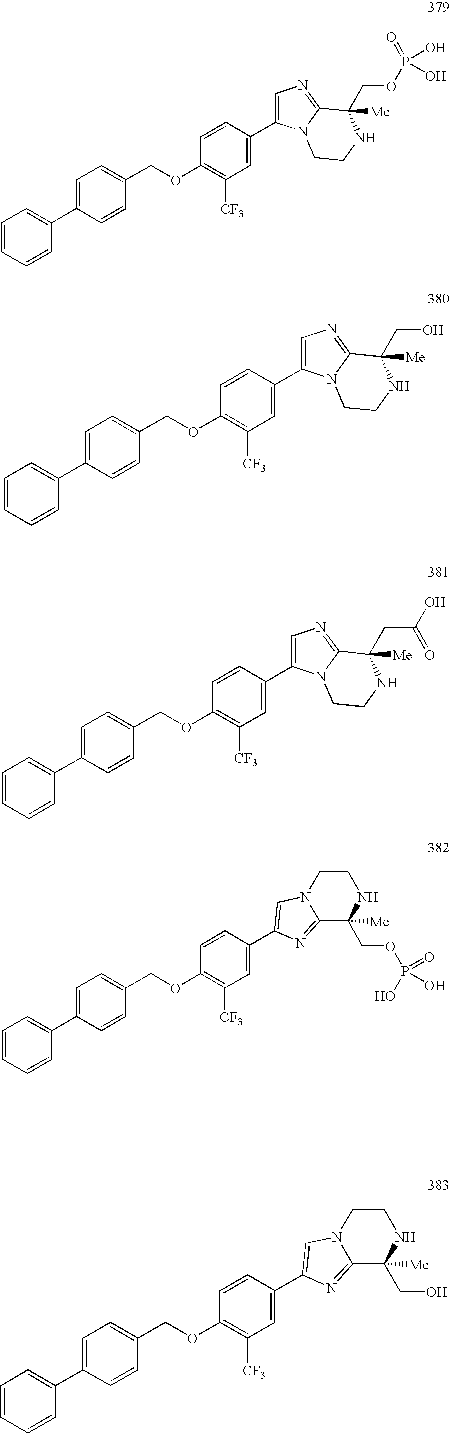

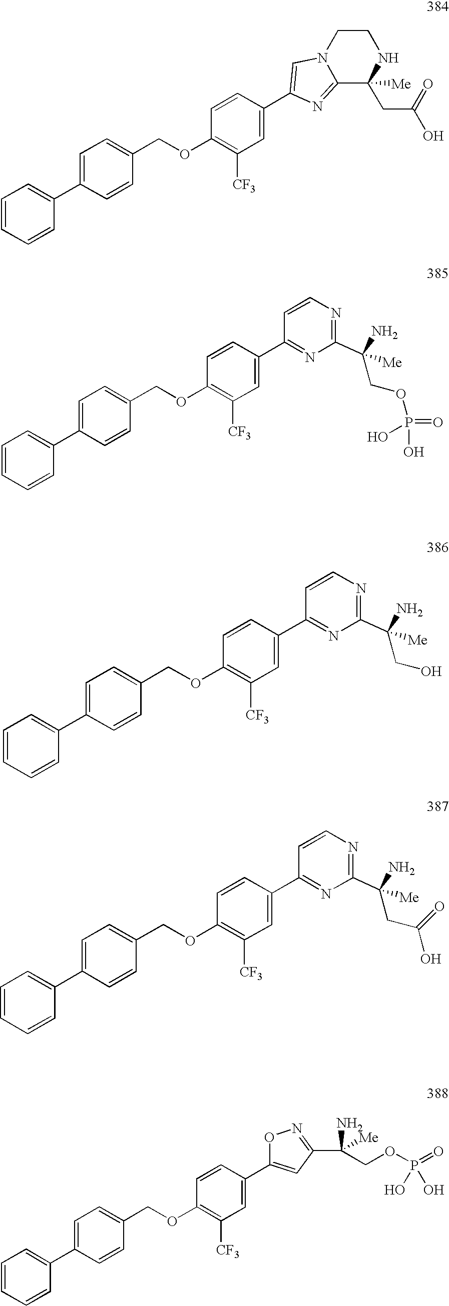

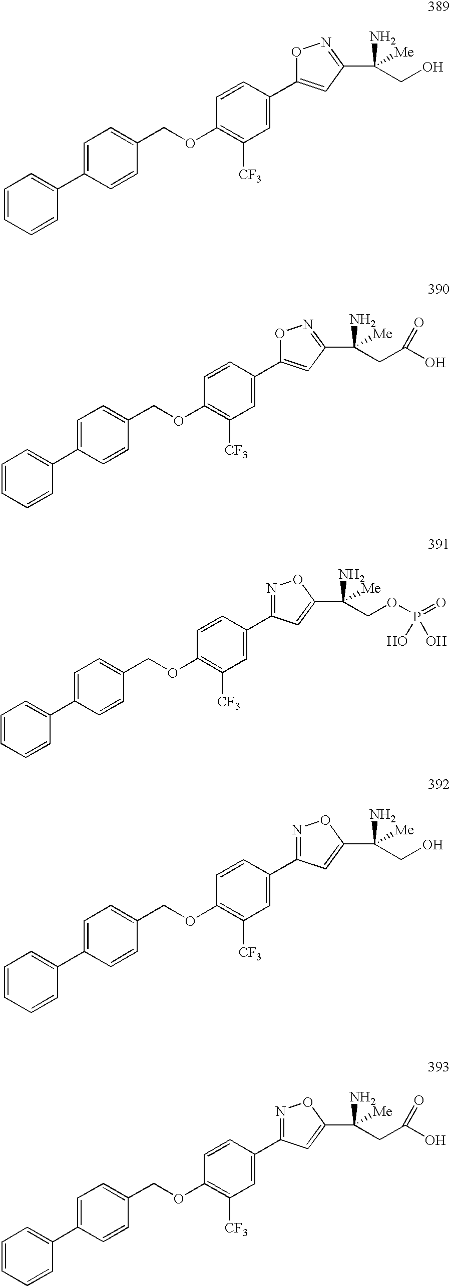

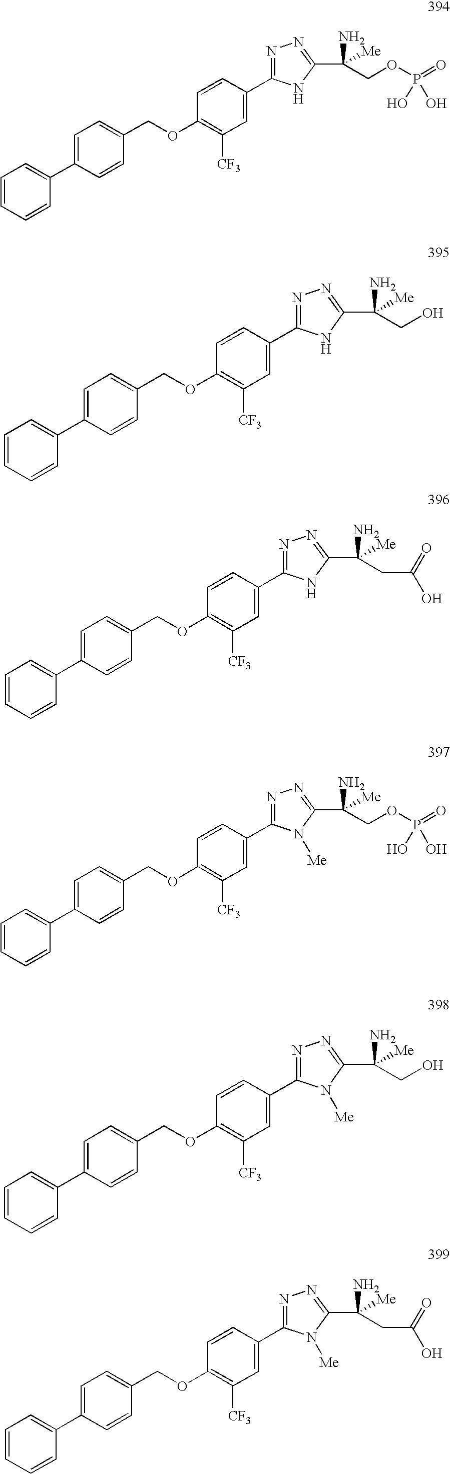

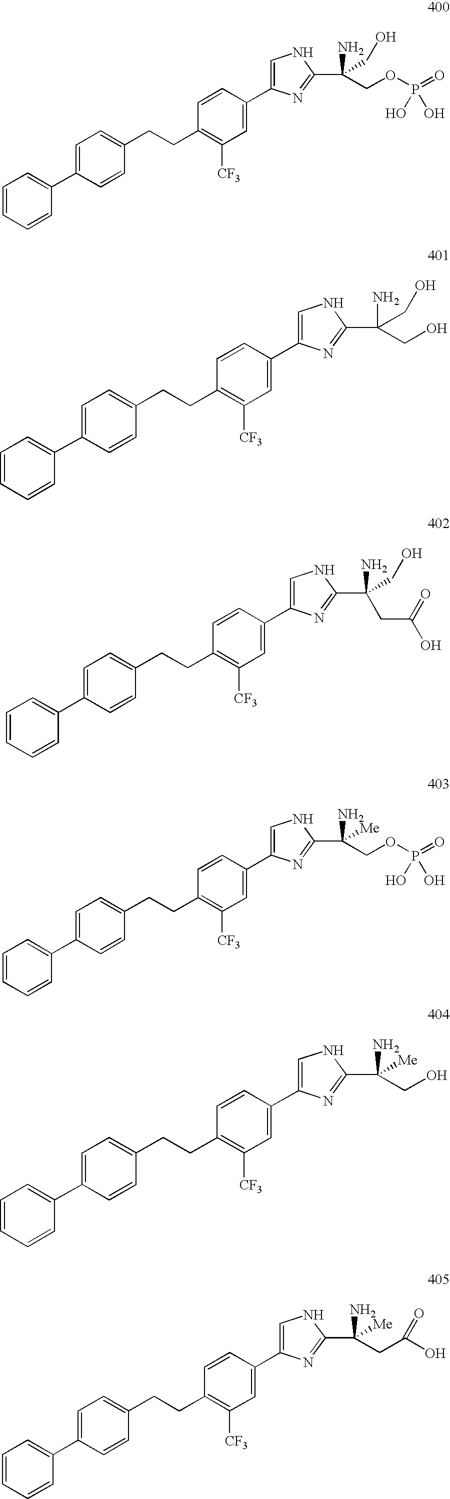

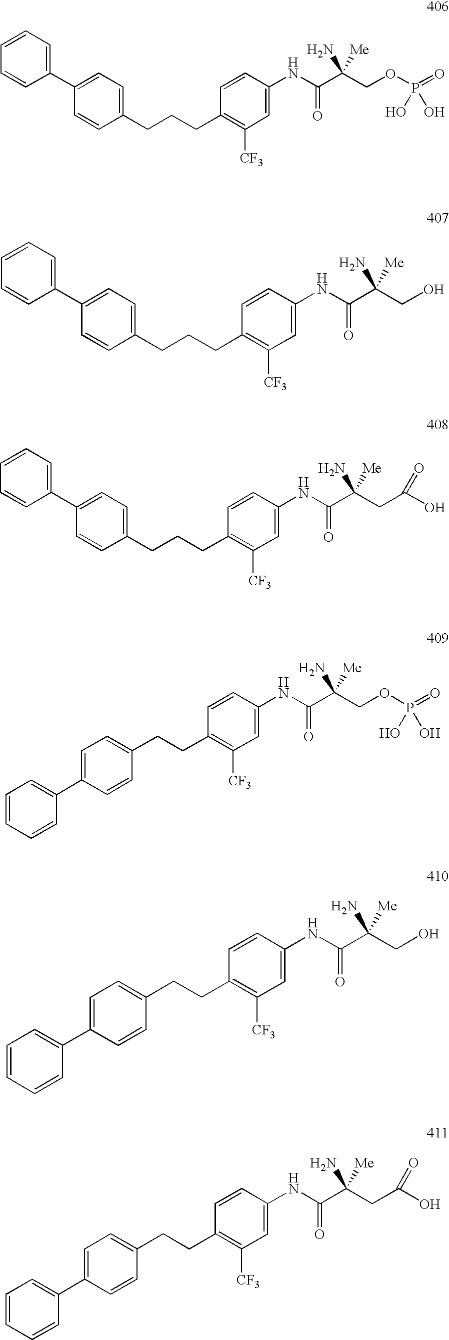

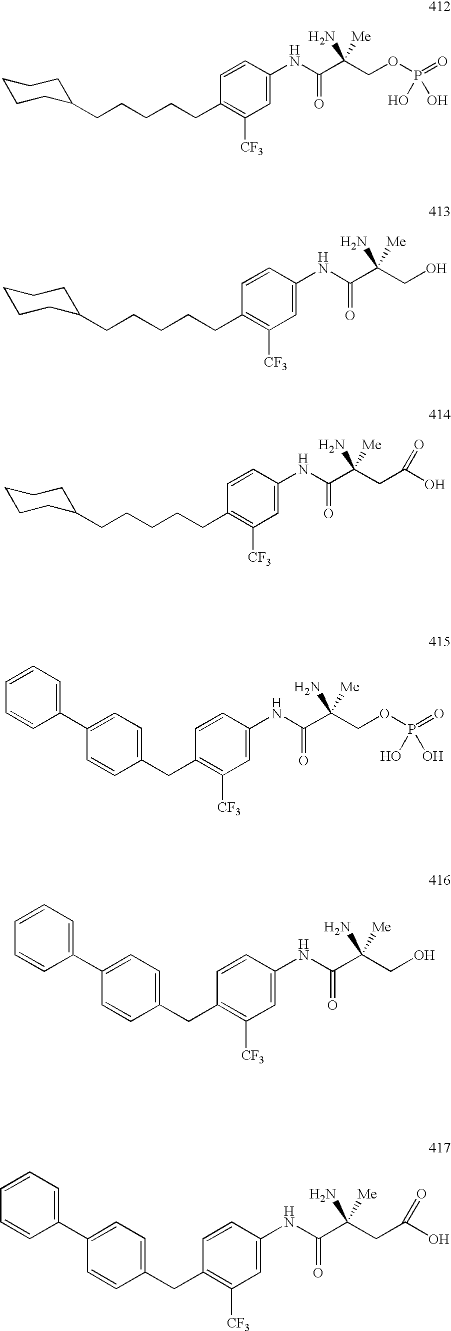

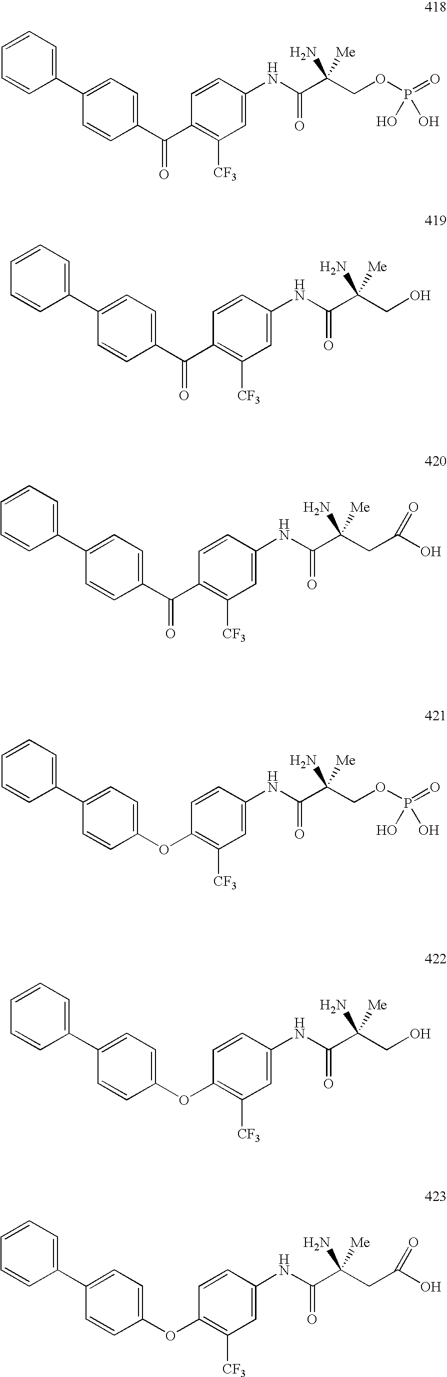

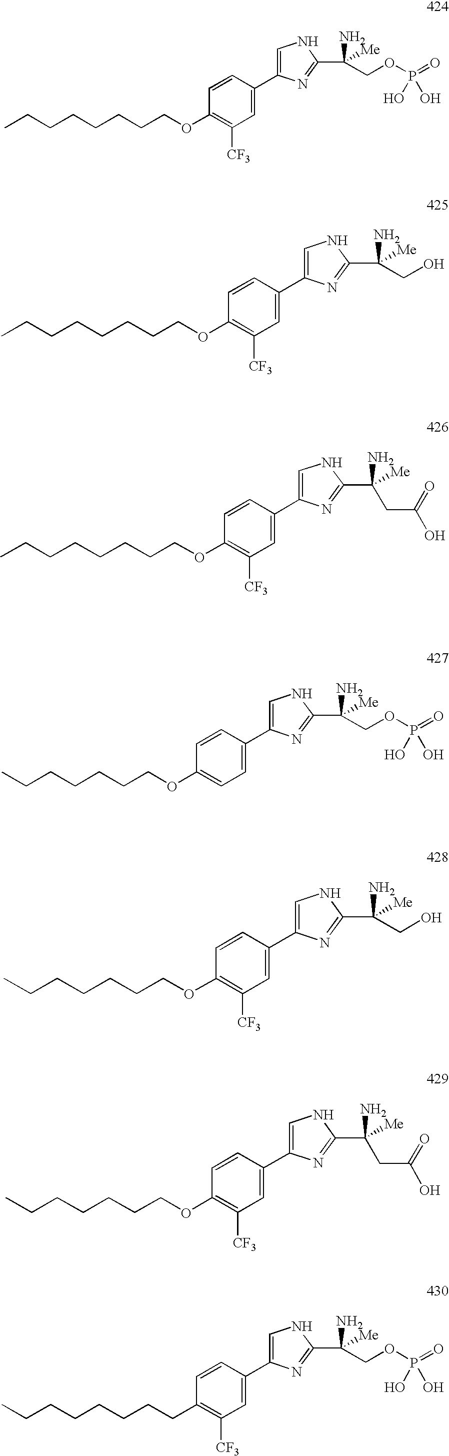

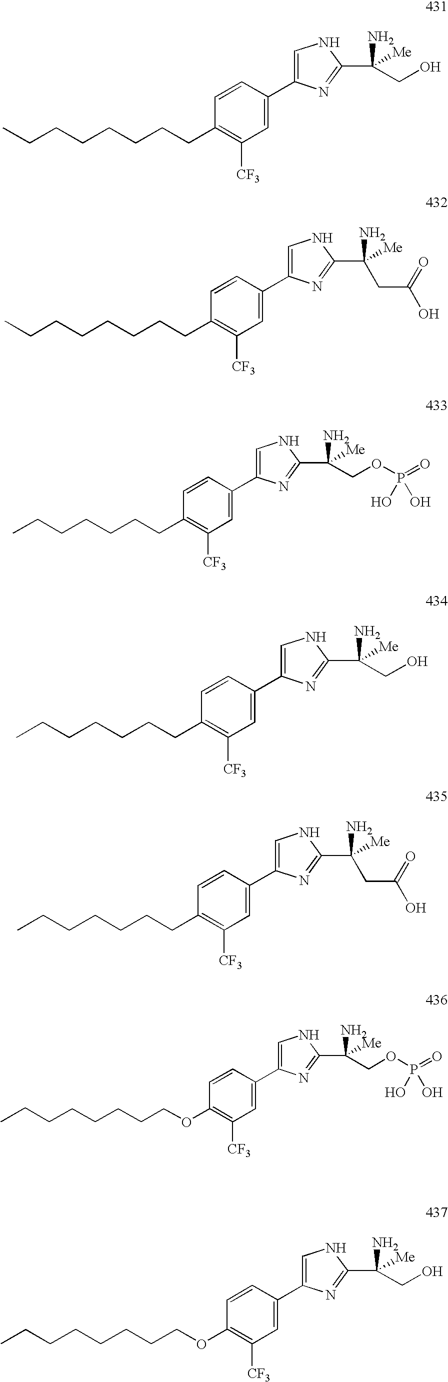

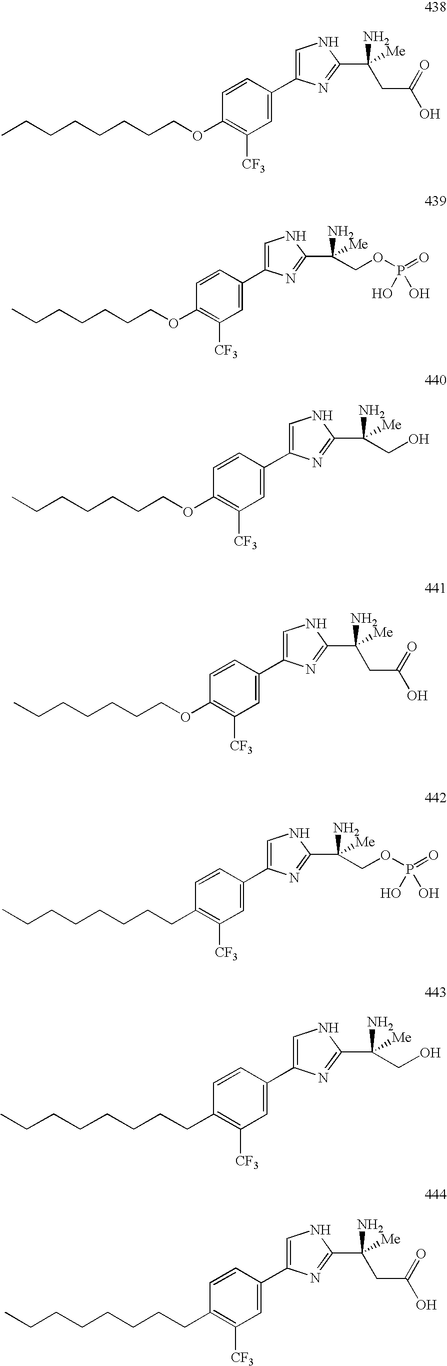

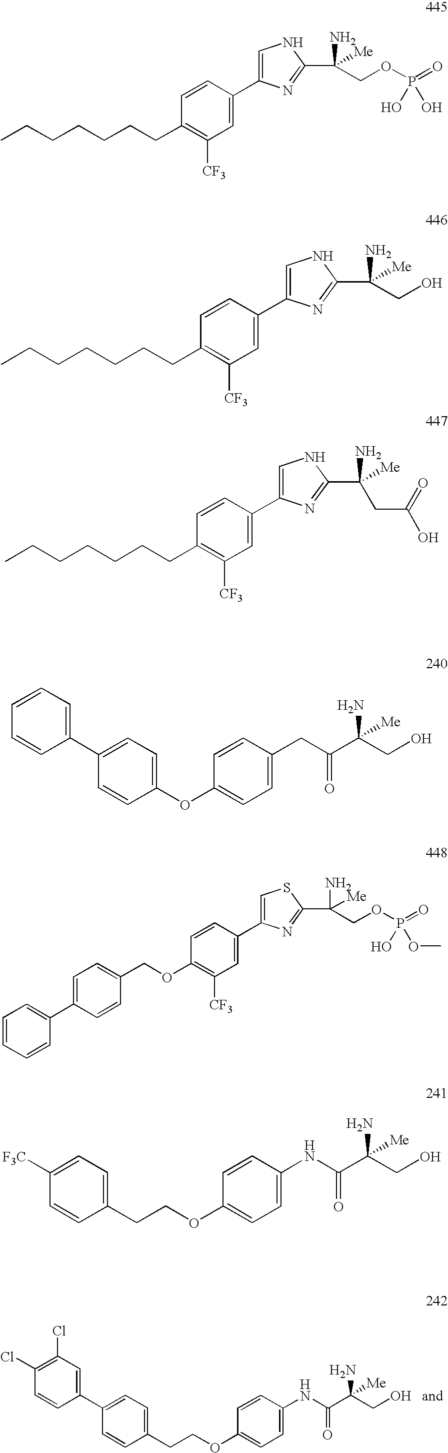

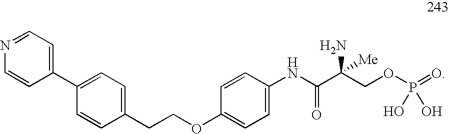

54. A compound of Formula XVII: 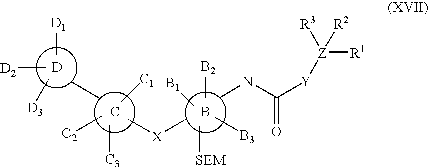
wherein:
SEM represents a selectivity enhancing moiety;
rings B, C, D are independently selected from the group consisting of substituted or unsubstituted carbocyclic rings and substituted or unsubstituted heterocyclic rings, which may contain one or more heteroatoms and may be saturated or unsaturated;
B₁, B₂, B₃, C₁, C₂, C₃, D₁, D₂, and D₃ are each independently selected from the group consisting of hydrogen, halogen, cyano, straight chain or branched aliphatic, straight chain or branched alkoxy, straight chain or branched halo-alkyl, straight chain or branched halo-alkoxy, alkoxy-alkyl, hydroxyl-alkyl, carboxy-alkyl, alkyl-SO₂ alkylcarbonyl, thioether, alkylsulfonyl, alkylcarbonylamino, alkylaminocarbonyl, alkyloxycarbonyl, alkylcarbonyloxy, substituted or unsubstituted aryl, substituted or unsubstituted heteroaryl, —OH, —C(O)-alkyl, —C(O)-halo-alkyl, —C(O)O-alkyl, —C(O)O-halo-alkyl, —CONH₂, —CONH-alkyl, —CON-dialkyl, —CONH-halo-alkyl, —CON-halo-dialkyl, -alkyl-CONH-alkyl, -alkyl-CON-dialkyl, halo-alkyl-CONH- alkyl, halo-alkyl-CONH-halo-alkyl, alkyl-CONH-halo-dialkyl, -alkyl-hydroxyl, -alkyl-hydroxyl-alkyl, -halo-alkyl-hydroxyl-alkyl, -alkyl-hydroxyl-halo-alkyl, -halo-alkyl-hydroxyl-halo-alkyl, substituted or unsubstituted alkyl-OR¹⁴, substituted or unsubstituted haloalkyl-OR⁴, —OR¹⁴, and N(R)R′; or taken together R^{Z} and B₃ may form a substituted or unsubstituted carbocyclic ring or substituted or unsubstituted heterocyclic ring, which may contain one or more heteroatoms and may be saturated or unsaturated; or taken together C₂ and D₂ may form a substituted or unsubstituted carbocyclic ring or substituted or unsubstituted heterocyclic ring, which may contain one or more heteroatoms and may be saturated or unsaturated;
R and R′ are each independently selected from the group consisting of hydrogen, cyano, straight chain or branched alkyl, straight chain or branched alkoxy, straight chain or branched halo-alkyl, straight chain or branched halo-alkoxy, alkoxy-alkyl, hydroxyl-alkyl, and carboxy-alkyl; or taken together R and R′ may form a substituted or unsubstituted carbocyclic ring or substituted or unsubstituted or heterocyclic ring, which may contain one or more heteroatoms and may be saturated or unsaturated; or taken together with the N to which they are attached R and R′ may form a moiety selected from the group consisting of substituted straight chain or cyclic guanyl, straight chain or cyclic guanidine, straight chain or cyclic urea, straight chain or cyclic thiourea, straight chain or cyclic carbamate, and straight chain or cyclic thiocarbamate;
Z is independently selected from the group consisting of C or N;
R′ is a phosphate derivative, a phosphate mimic or a phosphate precursor;
R² and R³ are each independently selected from the group consisting of hydrogen, hydroxyl, halogen, cyano, straight chain or branched alkyl, alkyl-OR⁹, halo-alkyl-OR⁹, alkoxy-OR⁹, alkyl-OC(O)R⁹, halo-alkyl-OC(O)R⁹, alkoxy-OC(O)R⁹, carbocyclic rings, heterocyclic rings which may contain one or more heteroatoms, alkyl-NR⁹R¹⁰, halo-alkyl-NR⁹R¹⁰, and alkoxy-NR⁹R¹⁰, all of which may be optionally substituted with OH, halogen, NHR⁹, NR⁹R¹⁰, straight chain or branched alkoxy, straight chain or branched halo-alkyl, straight chain or branched halo-alkoxy, alkoxy-alkyl, hydroxyl-alkyl, or carboxy-alkyl; or taken together R² and R³ may form a substituted or unsubstituted carbocyclic ring or a substituted or unsubstituted heterocyclic ring, which may contain one or more heteroatoms and may be saturated or unsaturated; or taken together R² and R^{Z} may form a substituted or unsubstituted C₄-C₁₀ fused carbocyclic ring or substituted or unsubstituted C₄-C₁₀ fused heterocyclic rings, which may contain one or more heteroatoms and may be saturated or unsaturated; R⁹ and R¹⁰ are independently selected from the group consisting of hydrogen, halogen, cyano, straight chain or branched alkyl, straight chain or branched alkoxy, straight chain or branched halo-alkyl, straight chain and branched halo-alkoxy, —C(O)alkyl, —C(O)NH-alkyl, —C(O)N-dialkyl, —C(O)aryl, —C(O)NH-aryl, —C(O)N-alkyl-aryl, —C(O)N-diaryl, —C(O)heteroaryl, —C(O)NH-heteroaryl, —C(O)N-carbocycle, substituted or unsubstituted carbocyclic rings, and substituted or unsubstituted heterocyclic rings, which may contain one or more heteroatoms and may be saturated or unsaturated; or taken together R⁹ and R¹⁰ may form a substituted or unsubstituted carbocyclic ring or a substituted or unsubstituted heterocyclic ring, which may contain one or more heteroatoms and may be saturated or unsaturated;
Y is independently selected from the group consisting of (CR¹¹R¹²)ₙ and (CR¹¹R¹²)ₙNR¹³;
R¹¹, R¹², and R¹³ are independently selected from the group consisting of hydrogen, halogen, cyano, and straight chain or branched alkyl, all of which may be optionally substituted with OH, halogen, straight chain or branched alkoxy, straight chain or branched halo-alkyl, and straight chain and branched halo-alkoxy; or R¹³ may form a 3-8-membered ring together with either R¹¹ or R¹² and the atom to which they are attached;
n is an integer from 0 to 3;
X is selected from the group consisting of 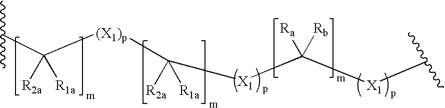
wherein each m is independently selected from an integer between 0 and 6; each p is independently selected from 0 or 1; each X₁ is independently selected from the group consisting of CR¹⁴R¹⁵, NR¹⁴, S, and O, —S(O), —S(O)₂, —OS(O)₂, —OS(O)₂O—, —C(O), C(OH), —C(O)O—, a substituted or unsubstituted aromatic, a substituted or unsubstituted heteroaromatic, and any combination thereof, in any orientation; each Rₐ and R_{b} are independently selected from the group consisting of hydrogen, cyano, halogen, alkyl, halo-alkyl, —OH, —CO—, straight chain or branched alkoxy, straight chain or branched halo-alkyl, straight chain or branched halo-alkoxy, alkoxy-alkyl, hydroxyl-alkyl, -alkyl-hydroxyl, -alkyl-hydroxyl-alkyl, -halo-alkyl-hydroxyl-alkyl, -alkyl-hydroxyl-halo-alkyl, -halo-alkyl-hydroxyl-halo-alkyl, carboxy-alkyl, alkyl-SO₂, alkylcarbonyl, thioether, alkylsulfonyl, alkylcarbonylamino, alkylaminocarbonyl, alkyloxycarbonyl, alkylcarbonyloxy, substituted or unsubstituted aryl, and substituted or unsubstituted heteroaryl, all of which may be optionally substituted with OH, halogen, e.g., fluoro, straight chain or branched alkoxy, straight chain or branched halo-alkyl, straight chain and branched halo-alkoxy, alkoxy-alkyl, hydroxyl-alkyl, carboxy-alkyl, substituted or unsubstituted carbocyclic rings, and substituted or unsubstituted heterocyclic rings, which may contain one or more heteroatoms and may be saturated or unsaturated; or each Rₐ and R_{b} may form a 3-10-membered ring together with the carbon to which they are both attached;
each R₁ₐ and R₂ₐ are independently selected from the group consisting of hydrogen, cyano, halogen, alkyl, halo-alkyl, —OH, —CO—, straight chain or branched alkoxy, straight chain or branched halo-alkyl, straight chain or branched halo-alkoxy, alkoxy-alkyl, hydroxyl-alkyl, -alkyl-hydroxyl, -alkyl-hydroxyl-alkyl, -halo-alkyl-hydroxyl-alkyl, -alkyl-hydroxyl-halo-alkyl, -halo-alkyl-hydroxyl-halo-alkyl, carboxy-alkyl, alkyl-SO₂, alkylcarbonyl, thioether, alkylsulfonyl, alkylcarbonylamino, alkylaminocarbonyl, alkyloxycarbonyl, alkylcarbonyloxy, substituted or unsubstituted aryl, and substituted or unsubstituted heteroaryl, all of which may be optionally substituted with OH, halogen, e.g., fluoro, straight chain or branched alkoxy, straight chain or branched halo-alkyl, straight chain and branched halo-alkoxy, alkoxy-alkyl, hydroxyl-alkyl, carboxy-alkyl, substituted or unsubstituted carbocyclic rings, and substituted or unsubstituted heterocyclic rings, which may contain one or more heteroatoms and may be saturated or unsaturated; or each Ria and R₂ₐ may form a 3-10-membered ring together with the carbon to which they are both attached;
each R¹⁴ and R¹⁵ is independently selected from the group consisting of hydrogen, halogen, cyano, straight chain or branched alkyl, straight chain or branched alkoxy, straight chain or branched halo-alkyl, straight chain or branched halo-alkoxy, alkoxy-alkyl, hydroxyl-alkyl, alkyl-SO₂, and carboxy-alkyl; or each R¹⁴ or R¹⁵ may form a 3-8-membered ring together with B₁, Rₐ, R_{b}, R₁ₐ, or R₂ₐ and the atoms to which they are attached; and
R^{Z} selected from the group consisting of hydrogen, cyano, straight chain or branched alkyl, cycloalkyl, straight chain or branched alkoxy, straight chain or branched halo-alkyl, halo-cycloalkyl, straight chain or branched halo-alkoxy, alkoxy-alkyl, hydroxyl-alkyl, alkyl-SO₂, and carboxy-alkyl; or R^{Z} may form a 3-8-membered ring together with B₁, R² or R³ and the atoms to which they are attached.

55. The compound of claim 54, wherein the total combination of each p and m is less than or equal to about 10.

56. A method for treating a subject suffering from a sphingosine 1-phosphate associated disorder, comprising administering to said subject an effective amount of a compound of claim 1 or 54, such that the subject is treated for a sphingosine 1-phosphate associated disorder.

57. A method for treating a subject suffering from a sphingosine 1-phosphate associated disorder, comprising administering to a subject a compound, such that the subject is treated for a sphingosine 1-phosphate associated disorder by a compound of claim 1 or 54.

58. A method of selectively treating a sphingosine 1-phosphate associated disorder, comprising administering to a subject an effective amount of a compound of claim 1 or 54, such that the subject is selectively treated for a sphingosine 1-phosphate associated disorder.

59. A method of selectively treating a sphingosine 1-phosphate associated disorder, comprising administering to a subject a compound, such that the subject is selectively treated for a sphingosine 1-phosphate associated disorder by a compound of claim 1 or 54.

60. The method of claim 58, wherein the sphingosine 1-phosphate associated disorder is a sphingosine 1-phosphate-(1)associated disorder.

61. The method of claim 60, wherein the sphingosine 1-phosphate-(1) associated disorder is an autoimmune disorder or condition associated with an overactive immune response.

62. A pharmaceutical composition comprising a therapeutically effective amount of a compound of claim 1 or 54, and a pharmaceutically acceptable carrier.
